SEQUENCE LISTING (33 sequences)

SEQ ID NO: 1 - protein, 562 aa, Artificial Sequence
  rPro-hCD (Rat 1-217/Human 237-537)-TEV-2Strep- 6His
  ADAMTS7 rat prodomain (wild-type) (1)..(217)
  ADAMTS7 human catalytic domain (218)..(518)
  TEV cleavage site, 2xStrep tag, and a His tag (519)..(562)
MHRGLNLLLILCALAPHVLGPASGLPTEGRAGLDIVHPVRVDAGGSFLSYELWPRVLRKRDVSAAQASSAFYQLQYQGRELLFNLTTNPYLLAPGFVSEIRRRSNLSNVHIQTSVPTCHLLGDVQDPELEGGFAAISACDGLRGVFQLSNEDYFIEPLDEVPAQPGHAQPHMVYKHKRSGQQDDSRTSGTCGVQGSPELKHQREHWEQRQQKRRQQRSVSKEKWVETLVVADAKMVEYHGQPQVESYVLTIMNMVAGLFHDPSIGNPIHITIVRLVLLEDEEEDLKITHHADNTLKSFCKWQKSINMKGDAHPLHHDTAILLTRKDLCAAMNRPCETLGLSHVAGMCQPHRSCSINEDTGLPLAFTVAHELGHSFGIQHDGSGNDCEPVGKRPFIMSPQLLYDAAPLTWSRCSRQYITRFLDRGWGLCLDDPPAKDIIDFPSVPPGVLYDVSHQCRLQYGAYSAFCEDMDNVCHTLWCSVGTTCHSKLDAAVDGTRCGENKWCLSGECVPVGFRPEAVGSENLYFQSGWSHPQFEKGGGSGGGSGGGSWSHPQFEKHHHHHH

SEQ ID NO: 2 - protein, 562 aa, Artificial Sequence
  rPro-hCD-FM2 (Rat 1-217/Human 237-537 FM2 (Q216K))-TEV-2Strep-6His
  ADAMTS7 rat prodomain (furin mutant Q216K) (1)..(217)
  ADAMTS7 human catalytic domain (218)..(518)
  TEV cleavage site, 2xStrep tag, and a His tag (519)..(562)
MHRGLNLLLILCALAPHVLGPASGLPTEGRAGLDIVHPVRVDAGGSFLSYELWPRVLRKRDVSAAQASSAFYQLQYQGRELLFNLTTNPYLLAPGFVSEIRRRSNLSNVHIQTSVPTCHLLGDVQDPELEGGFAAISACDGLRGVFQLSNEDYFIEPLDEVPAQPGHAQPHMVYKHKRSGQQDDSRTSGTCGVQGSPELKHQREHWEQRQQKRRQKRSVSKEKWVETLVVADAKMVEYHGQPQVESYVLTIMNMVAGLFHDPSIGNPIHITIVRLVLLEDEEEDLKITHHADNTLKSFCKWQKSINMKGDAHPLHHDTAILLTRKDLCAAMNRPCETLGLSHVAGMCQPHRSCSINEDTGLPLAFTVAHELGHSFGIQHDGSGNDCEPVGKRPFIMSPQLLYDAAPLTWSRCSRQYITRFLDRGWGLCLDDPPAKDIIDFPSVPPGVLYDVSHQCRLQYGAYSAFCEDMDNVCHTLWCSVGTTCHSKLDAAVDGTRCGENKWCLSGECVPVGFRPEAVGSENLYFQSGWSHPQFEKGGGSGGGSGGGSWSHPQFEKHHHHHH

SEQ ID NO: 3 - protein, 573 aa, Artificial Sequence
  rADAMTS7 (Rat 1-575)-Flag
MPCAQGNWMAKLSMVAQLLNFGAFCHGRQAQPWPVRFPDPKQEHFIKSLPEYHIVSPVQVDASGHFLSYGLHHPVTGSRKKRAAGGSGDQVYYRISHEEKNLFFNLTVNWEFLSNGYVVERRYGNLSHVKMAASSGQPCHLRGTVLQQGPTIRMGTAALSACQGLTGFFHLPHGDFFIEPVKKHPLTEEGYQPHVIYRRQSYRVPETKEPTCGLKDSLDNSVKQELQREKWERKNWPSRSLSRRSISKERWVETLVVADTKMIEYHGSENVESYILTIMNMVTGLFHNPSIGNAIHIVVVRLILLEEEEQGLKIVHHAEKTLSSFCKWQKSINPKSDLNPVHHDVAVLLTRKDICAGFNRPCETLGLSHLSGMCQPHRSCNINEDSGLPLAFTIAHELGHSFGIQHDGKENDCEPVGRHPYIMSRQLQYDPTPLTWSKCSEEYITRFLDRGWGFCLDDIPKKKGLKSKVIAPGVIYDVHHQCQLQYGPNATFCQEVENVCQTLWCSVKGFCRSKLDAAADGTQCGEKKWCMAGKCITVGKKPESIPGGGGSDYKDHDGDYKDHDIDYKDDDDK

SEQ ID NO: 4 - protein, 16 aa, Artificial Sequence
  Test substrate for ADAMTS7/12 (HiLyteFluor- 488)-DELSSMVLELRGLRT-K(QXL520)-NH2
DELSSMVLELRGLRTK

SEQ ID NO: 5 - protein, 16 aa, Artificial Sequence
  Test Substrate for ADAMTS7/12 (HiLyteFluor-488)-SSMVLELRGLRTIVT-K(QXL520)-NH2
SSMVLELRGLRTIVTK

SEQ ID NO: 6 - protein, 16 aa, Artificial Sequence
  Test Substrate for ADAMTS7/12 (HiLyteFluor-488)-KVTEENKELANELRR-K(QXL520)-NH2
KVTEENKELANELRRK

SEQ ID NO: 7 - protein, 16 aa, Artificial Sequence
  Test Substrate for ADAMTS7/12 (HiLyteFluor-488)-EENKELANELRRPPL-K(QXL520)-NH2
EENKELANELRRPPLK

SEQ ID NO: 8 - protein, 13 aa, Artificial Sequence
  Test Substrate for ADAMTS7/12 (HiLyteFluor-488)-SSMVLELRGLRT-K(QXL520)-NH2
SSMVLELRGLRTK

SEQ ID NO: 9 - protein, 12 aa, Artificial Sequence
  Test Substrate for ADAMTS7/12 (Donor) GMQQSVRTGLPS (K-Quencher)
GMQQSVRTGLPS

SEQ ID NO: 10 - protein, 14 aa, Artificial Sequence
  Test Substrate for ADAMTS7/12 (Donor) SPGFRCEACPPGYS (K-Quencher)
SPGFRCEACPPGYS

SEQ ID NO: 11 - protein, 16 aa, Artificial Sequence
  Substrate for ADAMTS7 and for ADAMTS12 (HiLyteFluor-488)-DELSSMVLELRGLRT-K(QXL520)-E-NH2
DELSSMVLELRGLRTK

SEQ ID NO: 12 - protein, 16 aa, Artificial Sequence
  Test Substrate for ADAMTS7/12 (HiLyteFluor-488)-DELSSMVLELRGLRT-K(QXL520)-K-NH2
DELSSMVLELRGLRTK

SEQ ID NO: 13 - protein, 16 aa, Artificial Sequence
  Test Substrate for ADAMTS7/12 (HiLyteFluor-488)-DELSSMVLELRGLRT-K(QXL520)-OH
DELSSMVLELRGLRTK

SEQ ID NO: 14 - protein, 10 aa, Artificial Sequence
  Substrate for ADAMTS4 and for ADAMTS5 Dabcyl-EEVKAKVQPY-Glu(Edans)-NH2
EEVKAKVQPY

SEQ ID NO: 15 - protein, 573 aa, Artificial Sequence; identical to SEQ ID NO: 3
  rPro-hCD for ADAMTS12

SEQ ID NO: 16 - protein, 14 aa, Artificial Sequence
  ADAMTS Catalytic Motif Consensus Sequence
  misc_feature (3)..(4): Xaa can be any naturally occurring amino acid
  misc_feature (6)..(6): Xaa can be any naturally occurring amino acid
  misc_feature (12)..(12): Xaa can be any naturally occurring amino acid
HEXXHXBGNSBXHD

SEQ ID NO: 17 - protein, 7 aa, Artificial Sequence
  Substrate for MMP12 Mca-Pro-Leu-Gly-Leu-Glu- Glu-Ala-Dap(Dnp)-NH2
PLGLEEA

SEQ ID NO: 18 - protein, 8 aa, Artificial Sequence
  Substrate for MMP15 MCA-Lys-Pro-Leu-Gly-Leu- DPA-Ala-Arg-NH2
  X (6)..(6): DPA
KPLGLXAR

SEQ ID NO: 19 - protein, 7 aa, Artificial Sequence
  Substrate for MMP2 MCA-Pro-Leu-Gly-Leu-DPA- Ala-Arg-NH2
  X (5)..(5): DPA
PLGLXAR

SEQ ID NO: 20 - protein, 12 aa, Artificial Sequence
  Substrate for ADAM17 Mca-Pro-Leu-Ala-Gln-Ala-Val-Dap(Dnp)-Arg-Ser-Ser-Ser-Arg-NH2
  X (7)..(7): Dap(Dnp)
PLAQAVXRSSSR

SEQ ID NO: 21 - protein, 1686 aa, Artificial Sequence
  Human ADAMTS7 from Q9UKP4
MPGGPSPRSPAPLLRPLLLLLCALAPGAPGPAPGRATEGRAALDIVHPVRVDAGGSFLSYELWPRALRKRDVSVRRDAPAFYELQYRGRELRFNLTANQHLLAPGFVSETRRRGGLGRAHIRAHTPACHLLGEVQDPELEGGLAAISACDGLKGVFQLSNEDYFIEPLDSAPARPGHAQPHVVYKRQAPERLAQRGDSSAPSTCGVQVYPELESRRERWEQRQQWRRPRLRRLHQRSVSKEKWVETLVVADAKMVEYHGQPQVESYVLTIMNMVAGLFHDPSIGNPIHITIVRLVLLEDEEEDLKITHHADNTLKSFCKWQKSINMKGDAHPLHHDTAILLTRKDLCAAMNRPCETLGLSHVAGMCQPHRSCSINEDTGLPLAFTVAHELGHSFGIQHDGSGNDCEPVGKRPFIMSPQLLYDAAPLTWSRCSRQYITRFLDRGWGLCLDDPPAKDIIDFPSVPPGVLYDVSHQCRLQYGAYSAFCEDMDNVCHTLWCSVGTTCHSKLDAAVDGTRCGENKWCLSGECVPVGFRPEAVDGGWSGWSAWSICSRSCGMGVQSAERQCTQPTPKYKGRYCVGERKRFRLCNLQACPAGRPSFRHVQCSHFDAMLYKGQLHTWVPVVNDVNPCELHCRPANEYFAEKLRDAVVDGTPCYQVRASRDLCINGICKNVGCDFEIDSGAMEDRCGVCHGNGSTCHTVSGTFEEAEGLGYVDVGLIPAGAREIRIQEVAEAANFLALRSEDPEKYFLNGGWTIQWNGDYQVAGTTFTYARRGNWENLTSPGPTKEPVWIQLLFQESNPGVHYEYTIHREAGGHDEVPPPVFSWHYGPWTKCTVTCGRGVQRQNVYCLERQAGPVDEEHCDPLGRPDDQQRKCSEQPCPARWWAGEWQLCSSSCGPGGLSRRAVLCIRSVGLDEQSALEPPACEHLPRPPTETPCNRHVPCPATWAVGNWSQCSVTCGEGTQRRNVLCTNDTGVPCDEAQQPASEVTCSLPLCRWPLGTLGPEGSGSGSSSHELFNEADFIPHHLAPRPSPASSPKPGTMGNAIEEEAPELDLPGPVFVDDFYYDYNFINFHEDLSYGPSEEPDLDLAGTGDRTPPPHSHPAAPSTGSPVPATEPPAAKEEGVLGPWSPSPWPSQAGRSPPPPSEQTPGNPLINFLPEEDTPIGAPDLGLPSLSWPRVSTDGLQTPATPESQNDFPVGKDSQSQLPPPWRDRTNEVFKDDEEPKGRGAPHLPPRPSSTLPPLSPVGSTHSSPSPDVAELWTGGTVAWEPALEGGLGPVDSELWPTVGVASLLPPPIAPLPEMKVRDSSLEPGTPSFPTPGPGSWDLQTVAVWGTFLPTTLTGLGHMPEPALNPGPKGQPESLSPEVPLSSRLLSTPAWDSPANSHRVPETQPLAPSLAEAGPPADPLVVRNAGWQAGNWSECSTTCGLGAVWRPVRCSSGRDEDCAPAGRPQPARRCHLRPCATWHSGNWSKCSRSCGGGSSVRDVQCVDTRDLRPLRPFHCQPGPAKPPAHRPCGAQPCLSWYTSSWRECSEACGGGEQQRLVTCPEPGLCEEALRPNTTRPCNTHPCTQWVVGPWGQCSGPCGGGVQRRLVKCVNTQTGLPEEDSDQCGHEAWPESSRPCGTEDCEPVEPPRCERDRLSFGFCETLRLLGRCQLPTIRTQCCRSCSPPSHGAPSRGHQRVARR

SEQ ID NO: 22 - protein, 1595 aa, Artificial Sequence
  Rat ADAMTS7 from Q1EHB3
MHRGLNLLLILCALAPHVLGPASGLPTEGRAGLDIVHPVRVDAGGSFLSYELWPRVLRKRDVSAAQASSAFYQLQYQGRELLFNLTTNPYLLAPGFVSEIRRRSNLSNVHIQTSVPTCHLLGDVQDPELEGGFAAISACDGLRGVFQLSNEDYFIEPLDEVPAQPGHAQPHMVYKHKRSGQQDDSRTSGTCGVQGSPELKHQREHWEQRQQKRRQQRSISKEKWVETLVVADSKMVEYHGQPQVESYVLTIMNMVAGLYHDPSIGNPIHITVVRLIILEDEEKDLKITHHADDTLKNFCRWQKNVNMKGDDHPQHHDTAILLTRKDLCATMNHPCETLGLSHVAGLCHPQLSCSVSEDTGLPLAFTVAHELGHSFGIQHDGTGNDCESIGKRPFIMSPQLLYDRGIPLTWSRCSREYITRFLDRGWGLCLDDRPSKGVINFPSVLPGVLYDVNHQCRLQYGPSSAYCEDVDNVCYTLWCSVGTTCHSKMDAAVDGTSCGKNKWCLNGECVPEGFQPETVDGGWSGWSAWSVCSRSCGVGVRSSERQCTQPVPKNKGKYCVGERKRYRLCNLQACPPDRPSFRHTQCSQFDSMLYKGKLHKWVPVLNDENPCELHCRPFNYSNREKLRDAVMDGTPCYQGRISRDICIDGICKKVGCDFELDSGAEEDRCGVCRGDGSTCHTVSRTFKEAEGMGYVDVGLIPAGAREILIEEVAEAANFLALRSEDPDKYFLNGGWTIQWNGDYQVAGTTFTYTRKGNWETLTSPGPTTEPVWIQLLFQERNPGVHYKYTIQRASHSEAQPPEFSWHYGPWSKCPVTCGTGVQRQSLYCMEKQAGIVDEGHCDHLSRPRDRKRKCNEEPCPARWWVGDWQPCSRSCGPGGFFRRAVFCTRSVGLDEQRALEPSACGHLPRPLAEIPCYHYVACPSSWGVGNWSQCSVTCGAGIRQRSVLCINNTGVPCDGAERPITETFCFLQPCQYSTYIVDTGASGSGSSSPELFNEVDFDPHQPVPRPSPASSPKPVSISNAIDEEDPELDPPGPVFVDDFYYDYNFINFHEDLSYGSFEESHSDLVDIGGQTVPPHIRPTEPPSDSPVPTAGAPGAEEEGIQGSWSPSPLLSEASHSPPVLLENTPVNPLANFLTEEESPIGAPELGLPSVSWPPASVDGMVTSVAPGNPDELLVREDTQSQPSTPWSDRNKLSKDGNPLGPTSPALPKSPFPTQPSSPSNSTTQASLSPDAVEVSTGWNVALDPVLEADLKPVHAPTDPGLLDQIQTPHTEGTQSPGLLPRPAQETQTNSSKDPAVQPLQPSLVEDGAPTDLLPAKNASWQVGNWSQCSTTCGLGAIWRLVRCSSGNDEDCTLSSRPQPARHCHLRPCAAWRAGNWSKCSRNCGGGSATRDVQCVDTRDLRPLRPFHCQPGPTKPPTRQLCGTQPCLPWYTSSWRECSEACGGGEQQRLVTCPEPGLCEESLRPNNTRPCNTHPCTQWVVGPWGQCSAPCGGGVQRRLVKCVNTQTGLAEEDSDLCSHEAWPESSRPCATEDCELVEPSRCERDRLPFNFCETLRLLGRCQLPTIRAQCCRSCPPLSRGVPSRGHQRVARR

SEQ ID NO: 23 - protein, 1657 aa, Artificial Sequence
  Mouse ADAMTS7 from Q68SA9
MHRGPSLLLILCALASRVLGPASGLVTEGRAGLDIVHPVRVDAGGSFLSYELWPRVLRKRDVSTTQASSAFYQLQYQGRELLFNLTTNPYLMAPGFVSEIRRHSTLGHAHIQTSVPTCHLLGDVQDPELEGGFAAISACDGLRGVFQLSNEDYFIEPLDGVSAQPGHAQPHVVYKHQGSRKQAQQGDSRPSGTCGMQVPPDLEQQREHWEQQQQKRRQQRSVSKEKWVETLVVADSKMVEYHGQPQVESYVLTIMNMVAGLFHDPSIGNPIHISIVRLIILEDEEKDLKITHHAEETLKNFCRWQKNINIKGDDHPQHHDTAILLTRKDLCASMNQPCETLGLSHVSGLCHPQLSCSVSEDTGMPLAFTVAHELGHSFGIQHDGTGNDCESIGKRPFIMSPQLLYDRGIPLTWSRCSREYITRFLDRGWGLCLDDRPSKDVIALPSVLPGVLYDVNHQCRLQYGSHSAYCEDMDDVCHTLWCSVGTTCHSKLDAAVDGTSCGKNKWCLKGECVPEGFQPEAVDGGWSGWSAWSDCSRSCGVGVRSSERQCTQPVPKNRGKYCVGERKRSQLCNLPACPPDRPSFRHTQCSQFDGMLYKGKLHKWVPVPNDDNPCELHCRPSNSSNTEKLRDAVVDGTPCYQSRISRDICLNGICKNVGCDFVIDSGAEEDRCGVCRGDGSTCQTVSRTFKETEGQGYVDIGLIPAGAREILIEEVAEAANFLALRSEDPDKYFLNGGWTIQWNGDYRVAGTTFTYARKGNWENLTSPGPTSEPVWIQLLFQEKNPGVHYQYTIQRDSHDQVRPPEFSWHYGPWSKCTVTCGTGVQRQSLYCMERQAGVVAEEYCNTLNRPDERQRKCSEEPCPPRWWAGEWQPCSRSCGPEGLSRRAVFCIRSMGLDEQRALELSACEHLPRPLAETPCNRHVICPSTWGVGNWSQCSVTCGAGIRQRSVLCINNTDVPCDEAERPITETFCFLQPCQYPMYIVDTGASGSGSSSPELFNEVDFIPNQLAPRPSPASSPKPVSISNAIDEEELDPPGPVFVDDFYYDYNFINFHEDLSYGSFEEPHPDLVDNGGWTAPPHIRPTESPSDTPVPTAGALGAEAEDIQGSWSPSPLLSEASYSPPGLEQTSINPLANFLTEEDTPMGAPELGFPSLPWPPASVDDMMTPVGPGNPDELLVKEDEQSPPSTPWSDRNKLSTDGNPLGHTSPALPQSPIPTQPSPPSISPTQASPSPDVVEVSTGWNAAWDPVLEADLKPGHGELPSTVEVASPPLLPMATVPGIWGRDSPLEPGTPTFSSPELSSQHLKTLTMPGTLLLTVPTDLRSPGPSGQPQTPNLEGTQSPGLLPTPARETQTNSSKDPEVQPLQPSLEEDGDPADPLPARNASWQVGNWSQCSTTCGLGAIWRLVSCSSGNDEDCTLASRPQPARHCHLRPCAAWRTGNWSKCSRNCGGGSSTRDVQCVDTRDLRPLRPFHCQPGPTKPPNRQLCGTQPCLPWYTSSWRECSEACGGGEQQRLVTCPEPGLCEESLRPNNSRPCNTHPCTQWVVGPWGQCSAPCGGGVQRRLVRCVNTQTGLAEEDSDLCSHEAWPESSRPCATEDCELVEPPRCERDRLSFNFCETLRLLGRCQLPTIRAQCCRSCPPLSRGVPSRGHQRVARR

SEQ ID NO: 24 - protein, 1594 aa, Artificial Sequence
  Human ADAMTS12 from P58397
MPCAQRSWLANLSVVAQLLNFGALCYGRQPQPGPVRFPDRRQEHFIKGLPEYHVVGPVRVDASGHFLSYGLHYPITSSRRKRDLDGSEDWVYYRISHEEKDLFFNLTVNQGFLSNSYIMEKRYGNLSHVKMMASSAPLCHLSGTVLQQGTRVGTAALSACHGLTGFFQLPHGDFFIEPVKKHPLVEGGYHPHIVYRRQKVPETKEPTCGLKDSVNISQKQELWREKWERHNLPSRSLSRRSISKERWVETLVVADTKMIEYHGSENVESYILTIMNMVTGLFHNPSIGNAIHIVVVRLILLEEEEQGLKIVHHAEKTLSSFCKWQKSINPKSDLNPVHHDVAVLLTRKDICAGFNRPCETLGLSHLSGMCQPHRSCNINEDSGLPLAFTIAHELGHSFGIQHDGKENDCEPVGRHPYIMSRQLQYDPTPLTWSKCSEEYITRFLDRGWGFCLDDIPKKKGLKSKVIAPGVIYDVHHQCQLQYGPNATFCQEVENVCQTLWCSVKGFCRSKLDAAADGTQCGEKKWCMAGKCITVGKKPESIPGGWGRWSPWSHCSRTCGAGVQSAERLCNNPEPKFGGKYCTGERKRYRLCNVHPCRSEAPTFRQMQCSEFDTVPYKNELYHWFPIFNPAHPCELYCRPIDGQFSEKMLDAVIDGTPCFEGGNSRNVCINGICKMVGCDYEIDSNATEDRCGVCLGDGSSCQTVRKMFKQKEGSGYVDIGLIPKGARDIRVMEIEGAGNFLAIRSEDPEKYYLNGGFIIQWNGNYKLAGTVFQYDRKGDLEKLMATGPTNESVWIQLLFQVTNPGIKYEYTIQKDGLDNDVEQQMYFWQYGHWTECSVTCGTGIRRQTAHCIKKGRGMVKATFCDPETQPNGRQKKCHEKACPPRWWAGEWEACSATCGPHGEKKRTVLCIQTMVSDEQALPPTDCQHLLKPKTLLSCNRDILCPSDWTVGNWSECSVSCGGGVRIRSVTCAKNHDEPCDVTRKPNSRALCGLQQCPSSRRVLKPNKGTISNGKNPPTLKPVPPPTSRPRMLTTPTGPESMSTSTPAISSPSPTTASKEGDLGGKQWQDSSTQPELSSRYLISTGSTSQPILTSQSLSIQPSEENVSSSDTGPTSEGGLVATTTSGSGLSSSRNPITWPVTPFYNTLTKGPEMEIHSGSGEEREQPEDKDESNPVIWTKIRVPGNDAPVESTEMPLAPPLTPDLSRESWWPPFSTVMEGLLPSQRPTTSETGTPRVEGMVTEKPANTLLPLGGDHQPEPSGKTANRNHLKLPNNMNQTKSSEPVLTEEDATSLITEGFLLNASNYKQLTNGHGSAHWIVGNWSECSTTCGLGAYWRRVECSTQMDSDCAAIQRPDPAKRCHLRPCAGWKVGNWSKCSRNCSGGFKIREIQCVDSRDHRNLRPFHCQFLAGIPPPLSMSCNPEPCEAWQVEPWSQCSRSCGGGVQERGVFCPGGLCDWTKRPTSTMSCNEHLCCHWATGNWDLCSTSCGGGFQKRTVQCVPSEGNKTEDQDQCLCDHKPRPPEFKKCNQQACKKSADLLCTKDKLSASFCQTLKAMKKCSVPTVRAECCFSCPQTHITHTQRQRRQRLLQKSKEL

SEQ ID NO: 25 - protein, 1599 aa, Artificial Sequence
  Rat ADAMTS12 from D3ZTJ3
MPCAQGNWMAKLSMVAQLLNFGAFCHGRQAQPWPVRFPDPKQEHFIKSLPEYHIVSPVQVDASGHFLSYGLHHPVTGSRKKRAAGGSGDQVYYRISHEEKNLFFNLTVNWEFLSNGYVVERRYGNLSHVKMAASSGQPCHLRGTVLQQGPTIRMGTAALSACQGLTGFFHLPHGDFFIEPVKKHPLTEEGYQPHVIYRRQSYRVPETKEPTCGLKDSLDNSVKQELQREKWERKNWPSRSLSRRSISKERWVETLVVADTKMVEYHGSENVESYILTIMNMVTGLFHNPSIGNAVHIVVVRLILLEEEEQGLKIVHHAEKTLSSFCKWQKSINPKSDLNPVHHDVAVLITRKDICAGVNRPCETLGLSQLSGMCQPHRSCNINEDSGLPLAFTIAHELGHSFGIQHDGKENDCEPVGRHPYIMSQQIQYDPTPLTWSKCSKEYITRFLDRGRGFCLDDVPRKKGLKSNVIAPGVIYDVHHQCQLQYGPNATFCQEVENVCQTLWCSVKGFCRSKLDAAADGTRCGEKKWCMAGKCITVGKKPESIPGGWGRWSPWSHCSRTCGAGAQSAERLCNNPEPKFGGKYCTGERKRYRLCNVHPCRSDTPTFRQMQCSEFDTVPYKNQFYRWFPVFNPAHPCELYCRPIDEQFSERMLEAVIDGTPCFEGGNSRNVCINGICKRVGCDYEIDSNATEDRCGVCLGDGSACQTVKKVFRQKEGSGYIDIGLIPKGARDIRVMEIKAAGNFLAIRSEDPEKYYLNGGFIIQWNGNYKLAGTVFQYDRKGDLERLMAPGPTNESVWLQLLFQVTNPGIKYEYTVRKDGLDNDVEKLLYFWQFGRWTECSVTCGTGIRRQTAHCVKKGHGIVKTTFCNPETQPSVRQKKCYEKDCPPRWWAGEWEACSMTCGPYGEKKRTVLCIQTMGSDEQALPATDCQHLLKPKTLVSCNRDILCPSDWTVGNWSECSVSCGGGVRIRSVTCAKNLNEPCDKTRKPNSRALCGLQQCPFSRRVLKPNKDTVPSGKNPTTSEHDHFKPIPASTSRPTPLSTPTVPESVSTSTPTINSLGSTITSQEEPDGIGWQNNSTQAEEDSHIPTSVGSTSQTPLTSWSWSMQPDDENVSSSAIGPTSESDFWATTSDSGLSSSNAMTWQVTPFYSTATTEPEVEIHSGSGEDSDQPLNKEENNSVLWNKIRVPERDAPMEMDAEIPLGPPPTSYVTEESSWPPFSTMMKSSLPAWSFKNETPRDEGMITEKSGNIPLPLGGDHQTTSPEKLGNNDQLASANSTNPTQGSGPVLTEEDASTLIEEGFLLNASNYKHLMKDHSPAHWIVGNWSKCSTTCGLGAYWRSVECSTGMNADCAAIQRPDPAKKCHLRPCAGWRVGNWSKCSRNCSGGFKIREVQCMDGVDHHRSLRPFHCQFLAGVPPPLSMSCNLEPCEEWKVEPWSQCSRSCGGGVQERGVFCPGGLCDWTKRPASTVPCNRHLCCHWATGNWELCTTSCGGGSQKRTVHCIPSENSTTEDQDQCFCDHQARPPEFQNCNQQACRKSADLTCTKDRLSTSFCQTLKSMKKCSVPSVRVQCCLSCPQTQSIHTQRQRKQQMLQNHDTL

SEQ ID NO: 26 - protein, 1600 aa, Artificial Sequence
  Mouse ADAMTS12 from Q811B3
MPCARGSWLAKLSIVAQLINFGAFCHGRQTQPWPVRFPDPRQEHFIKSLPEYHIVSPVQVDAGGHVLSYGLHHPVTSSRKKRAAGGSGDQLYYRISHEEKDLFFNLTVNWEFLSNGYVVEKRYGNLSHVKMVASSGQPCHLRGTVLQQGTTVGIGTAALSACQGLTGFFHLPHGDFFIEPVKKHPLTEEGSYPHVVYRRQSIRAPETKEPICGLKDSLDNSVKQELQREKWERKTLRSRSLSRRSISKERWVETLVVADTKTVEYHGSENVESYILTIMNMVTGLFHSPSIGNLVHIVVVRLILLEEEEQGLKIVHHAEKTLSSFCKWQKSINPKSDLNPVHHDVAVLITRKDICAGVNRPCETLGLSQLSGMCQPHRSCNINEDSGLPLAFTIAHELGHSFGIQHDGKENDCEPVGRHPYIMSQQIQYDPTPLTWSKCSKEYITRFLDRGRGFCLDDIPSKKGLKSNVIAPGVIYDVHHQCQLQYGPNATFCQEVENVCQTLWCSVKGFCRSKLDAAADGTRCGEKKWCMAGKCITVGKKPESIPGGWGRWSPWSHCSRTCGAGAQSAERLCNNPEPKFGGKYCTGERKRYRLCNVHPCRSDTPTFRQMQCSEFDTVPYKNQFYRWFPVFNAAHPCELYCRPIDEQFSERMLEAVIDGTPCFEGGNSRNVCINGICKRVGCDYEIDSNATEDRCGVCLGDGSACQTVKKLFRQKEGSGYVDIGLIPKGARDIRVMEIKAAGNFLAIRSEDPEKYYLNGGFIIQWNGNYKLAGTVFQYDRKGDLEKLIAPGPTNESVWLQLLFQVTNPGIKYEYTVRKDGLDNDVEKLLYFWQFGRWTECSVTCGTGIRRQAAHCVKKGHGIVKTTFCNPETQPSVRQKKCHEKDCPPRWWAGEWEACSTTCGPYGEKKRTVLCIQTMGSDEQALPATDCQHLLKPKALVSCNRDILCPSDWTVGNWSECSVSCGGGVRIRSVTCAKNLNEPCDKTRKPNSRALCGLQQCPFSRRVLKPNKDIAPSGKNQSTAEHDPFKPIPAPTSRPTPLSTPTVPESMSTSTPTINSLGSTIASQEDANGMGWQNNSTQAEEGSHFPTSSGSTSQVPVTSWSLSIQPDDENVSSSAIGPTSEGDFWATTTSDSGLSSSDAMTWQVTPFYSTMTTDPEVEIHSGSGEDSDQPLNKDKSNSVIWNKIGVPEHDAPMETDAELPLGPPPTSYMGEEPSWPPFSTKMEGSLPAWSFKNETPRDDGMIAEKSRKIPLPLAGDHHPATSEKLENHDKLALPNTTNPTQGFGPVLTEEDASNLIAEGFLLNASDYKHLMKDHSPAYWIVGNWSKCSTTCGLGAYWRSVECSSGVDADCTTIQRPDPAKKCHLRPCAGWRVGNWSKCSRNCSGGFKIREVQCMDSLDHHRSLRPFHCQFLAGAPPPLSMSCNLEPCGEWQVEPWSQCSRSCGGGVQERGVSCPGGLCDWTKRPATTVPCNRHLCCHWATGNWELCNTSCGGGSQKRTIHCIPSENSTTEDQDQCLCDHQVKPPEFQTCNQQACRKSADLTCLKDRLSISFCQTLKSMRKCSVPSVRAQCCLSCPQAPSIHTQRQRKQQLLQNHDML

SEQ ID NO: 27 - protein, 202 aa, Artificial Sequence
  Human ADAMTS7 Pro Domain (hADAMTS7_Pro)
RATEGRAALDIVHPVRVDAGGSFLSYELWPRALRKRDVSVRRDAPAFYELQYRGRELRFNLTANQHLLAPGFVSETRRRGGLGRAHIRAHTPACHLLGEVQDPELEGGLAAISACDGLKGVFQLSNEDYFIEPLDSAPARPGHAQPHVVYKRQAPERLAQRGDSSAPSTCGVQVYPELESRRERWEQRQQWRRPRLRRLHQR

SEQ ID NO: 28 - protein, 193 aa, Artificial Sequence
  Rat ADAMTS7 Pro Domain (rADAMTS7_Pro)
LPTEGRAGLDIVHPVRVDAGGSFLSYELWPRVLRKRDVSAAQASSAFYQLQYQGRELLFNLTTNPYLLAPGFVSEIRRRSNLSNVHIQTSVPTCHLLGDVQDPELEGGFAAISACDGLRGVFQLSNEDYFIEPLDEVPAQPGHAQPHMVYKHKRSGQQDDSRTSGTCGVQGSPELKHQREHWEQRQQKRRQQR

SEQ ID NO: 29 - protein, 301 aa, Artificial Sequence
  Human ADAMTS7 CD domain (hADAMTS7_CD)
SVSKEKWVETLVVADAKMVEYHGQPQVESYVLTIMNMVAGLFHDPSIGNPIHITIVRLVLLEDEEEDLKITHHADNTLKSFCKWQKSINMKGDAHPLHHDTAILLTRKDLCAAMNRPCETLGLSHVAGMCQPHRSCSINEDTGLPLAFTVAHELGHSFGIQHDGSGNDCEPVGKRPFIMSPQLLYDAAPLTWSRCSRQYITRFLDRGWGLCLDDPPAKDIIDFPSVPPGVLYDVSHQCRLQYGAYSAFCEDMDNVCHTLWCSVGTTCHSKLDAAVDGTRCGENKWCLSGECVPVGFRPEAV

SEQ ID NO: 30 - protein, 302 aa, Artificial Sequence
  Rat ADAMTS7 CD domain (rADAMTS7_CD)
SISKEKWVETLVVADSKMVEYHGQPQVESYVLTIMNMVAGLYHDPSIGNPIHITVVRLIILEDEEKDLKITHHADDTLKNFCRWQKNVNMKGDDHPQHHDTAILLTRKDLCATMNHPCETLGLSHVAGLCHPQLSCSVSEDTGLPLAFTVAHELGHSFGIQHDGTGNDCESIGKRPFIMSPQLLYDRGIPLTWSRCSREYITRFLDRGWGLCLDDRPSKGVINFPSVLPGVLYDVNHQCRLQYGPSSAYCEDVDNVCYTLWCSVGTTCHSKMDAAVDGTSCGKNKWCLNGECVPEGFQPETV

SEQ ID NO: 31 - protein, 562 aa, Artificial Sequence
  rPro-hCD (Rat 1-217/Human 237-537 (R58A/R60A/R217A))-TEV-2Strep-6His
MHRGLNLLLILCALAPHVLGPASGLPTEGRAGLDIVHPVRVDAGGSFLSYELWPRVLAKADVSAAQASSAFYQLQYQGRELLFNLTTNPYLLAPGFVSEIRRRSNLSNVHIQTSVPTCHLLGDVQDPELEGGFAAISACDGLRGVFQLSNEDYFIEPLDEVPAQPGHAQPHMVYKHKRSGQQDDSRTSGTCGVQGSPELKHQREHWEQRQQKRRQQASVSKEKWVETLVVADAKMVEYHGQPQVESYVLTIMNMVAGLFHDPSIGNPIHITIVRLVLLEDEEEDLKITHHADNTLKSFCKWQKSINMKGDAHPLHHDTAILLTRKDLCAAMNRPCETLGLSHVAGMCQPHRSCSINEDTGLPLAFTVAHELGHSFGIQHDGSGNDCEPVGKRPFIMSPQLLYDAAPLTWSRCSRQYITRFLDRGWGLCLDDPPAKDIIDFPSVPPGVLYDVSHQCRLQYGAYSAFCEDMDNVCHTLWCSVGTTCHSKLDAAVDGTRCGENKWCLSGECVPVGFRPEAVGSENLYFQSGWSHPQFEKGGGSGGGSGGGSWSHPQFEKHHHHHH

SEQ ID NO: 32 - protein, 196 aa, Artificial Sequence
  Mouse ADAMTS-7 prodomain (Mouse_Pro)
LVTEGRAGLDIVHPVRVDAGGSFLSYELWPRVLRKRDVSTTQASSAFYQLQYQGRELLFNLTTNPYLMAPGFVSEIRRHSTLGHAHIQTSVPTCHLLGDVQDPELEGGFAAISACDGLRGVFQLSNEDYFIEPLDGVSAQPGHAQPHVVYKHQGSRKQAQQGDSRPSGTCGMQVPPDLEQQREHWEQQQQKRRQQR

SEQ ID NO: 33 - protein, 45 aa, Artificial Sequence
  TSP-1 candidate region
CDELSSMVLELRGLRTIVTTLQDSIRKVTEENKELANELRRPPLC

(19) United States
(12) Patent Application Publication
(10) Pub. No.: US 2023/0027985 A1
(43) Pub. Date: Jan. 26, 2023

(54) SUBSTITUTED HYDANTOINAMIDES AS ADAMTS7 ANTAGONISTS

(71) Applicants: Bayer Aktiengesellschaft, Leverkusen (DE); The Broad Institute, Inc., Cambridge, MA (US)

(72) Inventors: Daniel MEIBOM, Wuppertal (DE); Yolanda CANCHO GRANDE, Leverkusen (DE); Pierre WASNAIRE, Düsseldorf (DE); Sarah Anna Liesa JOHANNES, Hilden (DE); Niels LINDNER, Wuppertal (DE); Kristin BEYER, Hilden (DE); Till FREUDENBERGER, Velbert (DE); Damian BROCKSCHNIEDER, Haan (DE); Ilka MATHAR, Düsseldorf (DE); Jürgen KLAR, Wuppertal (DE); Dmitry ZUBOV, Remscheid (DE); Dzianis MENSHYKAU, Düsseldorf (DE); Tanja KRAINZ, Cambridge, MA (US); Eric STEFAN, Cambridge, MA (US); Bryan MACDONALD, Cambridge, MA (US); Yi XING, Cambridge, MA (US); Nadine ELOWE, Cambridge, MA (US); Guzman SANCHEZ, Murcia (ES)

(73) Assignees: Bayer Aktiengesellschaft, Leverkusen (DE); The Broad Institute, Inc., Cambridge, MA (US)

(21) Appl. No.: 17/776,961

(22) PCT Filed: Nov. 12, 2020
(86) PCT No.: PCT/EP2020/081875

§ 371 Date: May 13, 2022

(30) Foreign Application Priority Data
Nov. 15, 2019 (EP) 19383013.0

(51) Int. Cl. C07D 403/14; C07D 409/14; C07D 417/14; C07D 401/14; C07D 403/12; A61K 45/06

(52) U.S. Cl. CPC C07D 403/14; C07D 409/14; C07D 417/14; C07D 401/14; C07D 403/12; A61K 45/06

(57) ABSTRACT

The application relates to substituted hydantoinamides of formula (I) as ADAMTS7 antagonists, to processes for their preparation, their use alone or in combination for the treatment or prophylaxis of diseases, in particular of cardiovascular diseases, including atherosclerosis, coronary artery disease (CAD), peripheral vascular disease (PAD), arterial occlusive disease or restenosis after angioplasty. R¹ is hydrogen, alkyl, cycloalkyl, heterocycloalkyl, 5- to 6-membered heteroaryl or phenyl; R² is hydrogen or alkyl; A is 5-membered heteroaryl; Z is 6- to 10-membered aryl or 5- to 10-membered heteroaryl; all groups being optionally substituted.

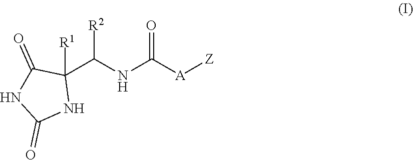

### FIELD OF THE INVENTION

The present application relates to substituted hydantoinamides, to processes for their preparation, their use alone or in combination for the treatment and/or prophylaxis of diseases as well as their use in the manufacturing-process of drugs which are used to treat and/or prophylactically treat diseases, in particular for the treatment and/or prophylaxis of heart diseases, vascular diseases, and/or cardiovascular diseases, including atherosclerosis, coronary artery disease (CAD), peripheral vascular disease (PAD)/arterial occlusive disease and/or restenosis after angioplasty (including the use of drug-coated or non drug-coated balloons and/or stent-implantation) and/or for the treatment and/or prophylaxis of lung diseases, inflammatory diseases, fibrotic diseases, metabolic diseases, cardiometabolic diseases and/or diseases/disease states affecting the kidneys and/or the central nervous and/or neurological system as well as gastrointestinal and/or urologic and/or ophthalmologic diseases/disease states.

### BACKGROUND

The pathogenesis of cardiovascular diseases and/or lung diseases as well as metabolic, inflammatory and/or vascular disease states including atherosclerosis and post-angioplasty restenosis is—at least in part—characterized by vascular remodeling (Wang et al., Sheng Li Xue Bao. 2010 Aug. 25; 62(4):285-94), a process which—amongst others—involves (metallo-) proteinases (Galis & Kathri, Circ Res., 2002 Feb. 22; 90(3):251-62). Accordingly, it may be beneficial to therapeutically interfere with these proteases in the context of the aforementioned diseases/diseases states. However, a lot of clinical trials having used pan-metalloproteinase inhibitors failed, in part due to severe side effects. Peterson et al. ascribe these negative outcomes to an inadequate assessment of the therapeutic index of the drugs tested, leading to the assumption that it is even more so important to explore the role of individual proteases (Wang et al., Sheng Li Xue Bao. 2010 Aug. 25; 62(4):285-94; Peterson, Cardiovasc. Res. 2006 Feb. 15; 69(3):677-87. Epub 2006 Jan. 17).

ADAMTS (a disintegrin and metalloproteinase with thrombospondin motifs) constitute a family of metalloproteases which consists of 19 secreted enzymes that have been described to be proteolytically active. ADAMTS play an important role in different processes such as assembly and degradation of extracellular matrix, angiogenesis, homeostasis, organogenesis, cancer, genetic disorders and arthritis (Zhang et al., Mediators Inflamm. 2015; 2015:801546. doi: 10.1155/2015/801546. Epub 2015 Nov. 30). The 19 ADAMTS enzymes (human) have been subclassified into 8 clusters.

The first 2 clusters comprise the aggrecanse- and proteoglycanase-groups (ADAMTS-1, -4, -5, -8, -15, and ADAMTS-9 & -20). The third cluster, consisting of pro-collagen N-propeptidases, is constituted by ADAMTS-2, -3 and -14 which are crucially involved in the maturation of collagen-fibrils. The only member of the fourth cluster is ADAMTS-13, a protease that cleaves von-Willebrand factor. ADAMTS-7 and ADAMTS-12 are members of the fifth cluster and they have been described to cleave cartilage oligomeric matrix protein (COMP). These two enzymes are unique inasmuch as chondroitinsulfate chains are part of these enzymes and thus confer a proteoglycan status to them (Kelwick et al., Genome Biol., 2015 May 30; 16:113. doi: 10.1186/s13059-015-0676-3). In human samples, the highest expression of ADAMTS7 was found in heart, kidney, sceletal muscle, liver and pancreas (Somerville, J. Biol. Chem., 2004 Aug. 20; 279(34):35159-75. Epub 2004 Jun. 10). Likewise, ADAMTS-7 was found to be expressed in bone, cartilage, synovium, tendons and ligaments. Additionally, it was detected in meniscus as well as fat (Liu, Nat Clin Pract Rheumatol. 2009 January; 5(1):38-45. doi: 10.1038/ncprheum0961).

The three clusters not yet referred to, contain two ADAMTS enzymes each (ADAMTS-6 and -10, ADAMTS-16 and -18, ADAMTS-17 and -19) and are characterized by the organization of the domains of their respective ADAMTS-members.

ADAMTS have been linked to divergent diseases: For example, mutations in ADAMTS13 have been associated with thrombotic, thrombozyopenic purpura (Levy et al., Nature. 2001 Oct. 4; 413(6855):488-94). ADAMTS-4 and -5 have been described to be responsible for the degradation of aggrecan in the cartilage of patients suffering from osteoarthritis (Malfait et al., J Biol Chem. 2002 Jun. 21; 277(25):22201-8. Epub 2002 Apr. 15).

The consensus sequence HEXXHXBG(/N/S)BXHD (SEQ ID NO: 16) is shared as catalytic motif by ADAMTS with the three histidine residues coordinating a Zn2+-ion (Kelwick et al., Genome Biol., 2015 May 30; 16:113. doi: 10.1186/s13059-015-0676-3). Different from other metalloproteinases, ADAMTS family members show a narrow substrate specificity which may make ADAMTS enzymes a potentially “safe” molecular target (Wu et al., Eur J Med Res, 2015 Mar. 21; 20:27. doi: 10.1186/540001-015-0118-4; Wang et al., Sheng Li Xue Bao. 2010 Aug. 25; 62(4):285-94).

In the context of the initially mentioned diseases/disease states ADAMTS-7 is of special interest since genome-wide association studies showed a correlation between ADAMTS-7 genetic variants and coronary artery disease (CAD). As opposed to genome-wide association studies showing variants in ADAMTS-7 to be associated with the prevalence of CAD and myocardial infarction (MI) (Chan et al., J Am Heart Assoc., 2017 Oct. 31; 6(11). pii: e006928. doi: 10.1161/JAHA.117.006928), Reilly et al. indentified variant in the ADAMTS-7 gene as being associated with CAD, however, not with MI (Reilly et al., Lancet. 2011 Jan. 29; 377(9763):383-92. doi: 10.1016/S0140-6736(10)61996-4. Epub 2011 Jan. 14). Further supporting a CAD-association, also a meta-analysis of 14 genome-wide association studies identified ADAMTS-7 as locus being linked to CAD (Schunkert et al., Nat. Genet. 2011 Mar. 6; 43(4):333-8. doi: 10.1038/ng.784).

One of the leading SNPs in the ADAMTS7 locus, rs3825807, shows an adenine (A)-to-guanine (G) exchange in the ADAMTS-7 gene with the G-allel being associated with a reduced risk for CAD. The above mentioned SNP (Single Nucleotide Polymorphism) leads to an amino acid exchange in the pro-domain of ADAMTS-7 and thereby has an impact on the maturation of ADAMTS-7. Furthermore, it has been described that the above mentioned SNP finds itself in a linkage disequilibrium with further SNPs within the ADAMTS-7 locus, such as rs1994016 and rs7178051 (Chan et al., J Am Heart Assoc., 2017 Oct. 31; 6(11). pii: e006928. doi: 10.1161/JAHA.117.006928). Supporting the aforementioned notions, rs3825807 has also been described to be associated with the susceptibility towards CAD in a chinese population and this association persisted even after correction for clinical co-variates (You et al., Mol Genet Genomics. 2016 February; 291(1):121-8. doi: 10.1007/s00438-015-1092-9. Epub 2015 Jul. 19).

Investigation of a potential relationship between plasma ADAMTS-7 levels and heart and/or lung function as well as analysis of ADAMTS-7 levels in patients suffering from an acute MI on the one hand showed that plasma ADAMTS-7 levels were higher in patients with a left ventricula ejection fraction (LVEF) below or equaling 35%, on the other hand revealed that plasma ADAMTS-7 levels positively correlated with, e.g., brain natriuretic peptide (BNP) and left ventricular end-systolic diameter (LVESD) while showing a negative correlation with the 6-min-walking distance (Wu et al., EurJ Med Res, 2015 Mar. 21; 20:27. doi: 10.1186/s40001-015-0118-4). Furthermore, high ADAMTS-7 levels correlated with a high lipid-content and accumulation of cluster of differentiation (CD) 68 and at the same time with a low smooth muscle cell—as well as a decreased collagen-content being characteristic for vulnerable plaques. Accordingly, ADAMTS-7-levels above median were associated with an increased risk for post-surgery cardiovascular events (Bengtsson et al., Sci Rep., 2017 Jun. 16; 7(1):3753. doi: 10.1038/s41598-017-03573-4).

Opposing the above mentioned studies, one study failed to show an association between initima- or media-thickness with the G-allel of the ADAMTS-7-SNP. However, a significant association was reported between the rs3825807 G-allel and a reduced thickness of the fibrous cap as well as a decreased portion of intimal α-actin accumulation. Notably, the G-allel of the ADAMTS-7-SNP was linked to a CAD-protective effect (16%-19% reduced risk to be taken ill with each allel after correction for age and gender). Further supporting former reports, no significant association was found between the G-allel and MI.

In a Hispanic subgroup, an analysis of the MESA (Multi-Ethnic Study of Artherosclerose)—cohort revealed an association of an ADAMTS-7-SNP different from the one described above and coronary artery calcification (Chan et al., J Am Heart Assoc., 2017 Oct. 31; 6(11). pii: e006928. doi: 10.1161/JAHA.117.006928).

Taken together, the well documented association with CAD allows the conclusion that ADAMTS-7 increases the risk for CAD via effects on atherosclerosis, potentially without influencing mechanisms which are supposed to favor plaque rupture or thrombosis which themselves are drivers for MI. This assumption is underlined by the observation that the protective G-allel of the ADAMTS-7 variant had no influence on overall mortality or MI (Chan et al., J Am Heart Assoc., 2017 Oct. 31; 6(11). pii: e006928. doi: 10.1161/JAHA.117.006928).

Experimentally, ADAMTS-7 has been described to be involved in the migration of smooth muscle cells (SMC) and the development of neointimal hyperplasia. Immunohistochemistry showed co-localisation of ADAMTS-7 with vascular smooth muscle cells (VSMC) within the neointima (Wang et al., Circ Res., 2009 Mar. 13; 104(5):688-98. doi: 10.1161/CIRCRESAHA.108.188425. Epub 2009 Jan. 22) and an increased expression of ADAMTS-7 in early- and late-stage human atherosclerotic plaques was described (Wang et al., Sheng Li Xue Bao. 2010 Aug. 25; 62(4):285-94).

Formation of a neointima is characterized by a media-to-intima migration of VSMC and their subsequent proliferation. Interestingly, migration as well as proliferation of VSMC were accelerated by ADAMTS-7 in vitro (Wang et al., Sheng Li Xue Bao. 2010 Aug. 25; 62(4):285-94). Along with this observation, overexpression of ADAMTS-7 led to an increased neointima formation in vivo, whereas a knockdown reduced/delayed injury-induced intimal hyperplasia (Wang et al, Circ Res., 2009 Mar. 13; 104(5):688-98. doi: 10.1161/CIRCRESAHA.108.188425. Epub 2009 Jan. 22).

Interesting to note is that pro-inflammatory cytokines such as TNF-α, IL-1β and PDGF-BB were able to induce ADAMTS-7 expression whereas TGF-β reduced its expression. Likewise, reactive oxygen species and H₂O₂ increased, while oxLDL and homocysteine did not change ADAMTS-7 expression in VSMC. All of the aforementioned factors are involved in vascular injury and may contribute to induction of ADAMTS-7 in injured arteries in vivo (Wang et al, Circ Res., 2009 Ma 13; 104(5):688-98. doi: 10.1161/CIRCRESAHA.108.188425. Epub 2009 Jan. 22).

Mechanistically, ADAMTS-7 may be involved in the pathogenesis of the above mentioned diseases/disease states such as, e.g., vascular disorders by cleaving COMP and thrombospondin-1 (TSP-1), by influencing migration and proliferation of VSMC and by regulating inflammatory cytokines (Zhang et al., Mediators Inflamm. 2015; 2015:801546. doi: 10.1155/2015/801546. Epub 2015 Nov. 30). In this context, it has been described that ADAMTS-7 directly binds to and cleaves COMP (Liu et al., FASEB J. 2006 May; 20(7):988-90. Epub 2006 Apr. 3) with the catalytic domain of ADAMTS-7 being responsible for COMP-cleavage (Liu, Nat Clin Pract Rheumatol., 2009 January; 5(1):38-45. doi: 10.1038/ncprheum0961). The disintegrin-like domain may be involved in the regulation of ADAMTS-7-activity by providing substrate-surfaces (Stanton et al., Biochim Biophys Acta., 2011 December; 1812(12):1616-29. doi: 10.1016/j.bbadis.2011.08.009. Epub 2011 Sep. 2). While ADAMTS-7 accelerates VMSC-migration via COMP-degradation, COMP has no effect on VSMC-proliferation. Interestingly, ADAMTS-7-mediated COMP-degradation in response to injury accelerated VSMC-migration and neointima hyperplasia. Taking into account that during restenosis (after vessel-wall injury) VSMC de-differentiate towards a synthetic phenotype, this may—in the context of the findings described above—lead to the hypothesis that COMP preserves a contractile phenotype in VSMC. Thus, ADAMTS-7 may constitute a new molecular target in conditions of atherosclerosis and/or restenosis after angioplasty (Wang et al., Sheng Li Xue Bao. 2010 Aug. 25; 62(4):285-94).

Furthermore it has been described that ADAMTS-7 is able to contribute to endothelial repair via direct binding to TSP-1, independent of COMP (Zhang et al., Mediators Inflamm. 2015; 2015:801546. doi: 10.1155/2015/801546. Epub 2015 Nov. 30). α2-macroglobulin has also been described to associate with ADAMTS-7 and to be its substrate (Luan et al., Osteoarthritis Cartilage. 2008 November; 16(11):1413-20. doi: 10.1016/j.joca.2008.03.017. Epub 2008 May 15).

In addition, it has been described that ADAMTS7 is involved in VSMC-calcification (Du et al., Arterioscler Thromb Vasc Biol., 2012 November; 32(11):2580-8. doi: 10.1161/ATVBAHA.112.300206. Epub 2012 Sep. 20) and that it contributes to oval cell activation as well as being an important regulator in the context of biliary fibrosis.

Furthermore, ADAMTS-7 seems to be involved in interactions of host and pathogen (Zhang et al., Mediators Inflamm. 2015; 2015:801546. doi: 10.1155/2015/801546. Epub 2015 Nov. 30.)

ADAMTS-7 and ADAMTS-12 were found to be significantly increased in cartilage and synovium of patients suffering from arthritis (Liu, Nat Clin Pract Rheumatol. 2009 January; 5(1):38-45. doi: 10.1038/ncprheum0961) and ADAMTS-7 could be detected in the urine of patients with different cancer-entities such as prostate and bladder cancer (Roy et al., Clin Cancer Res. 2008 Oct. 15; 14(20):6610-7. doi: 10.1158/1078-0432.CCR-08-1136).

Additionally, ADAMTS-7 expression was increased in patients with aortic aneurysms while expression of COMP was markedly reduced in this group of patients as compared to controls. Accordingly, increased expression of ADAMTS-7 and decreased levels of COMP seem to be associated with aortic aneurysms (Qin et al., Mol Med Rep., 2017 October; 16(4):5459-5463. doi: 10.3892/mmr.2017.7293. Epub 2017 Aug. 21).

New therapeutic concepts are necessary to improve the lifes/outcomes of patients who suffer from lung diseases, heart diseases, inflammatory diseases, fibrotic diseases, metabolic diseases, cardiometabolic diseases, vascular diseases and/or cardiovascular diseases, including atherosclerosis, coronary artery disease, peripheral vascular disease/arterial occlusive disease and/or restenosis after angioplasty (including the use of drug-coated or non drug-coated balloons and/or stent-implantation).

Likewise, identification of low molecular weight compounds for a preventive treatment or a treatment to slow progression of these diseases are conceivable and pursued.

### BRIEF DESCRIPTION OF THE FIGURES

FIGS. 1A, 1B 1C, 1D, 1E and 1F (consisting of panels A, B, and C) show comparisons of recombinantly expressed wild type (WT) human ADAMTS-7, WT rat ADAMTS-7, and hybrid ADAMTS-7. Lane numbers of the SDS PAGE correspond with annotated SEC fractions. FIG. 1A and FIG. 1B show size exclusion (SEC) profile, SDS-page, and western blot (WB) analysis of human ADAMTS-7 (hADAMTS-7) (1-537 with respect to SEQ ID NO: 21) from mammalian cell culture. FIG. 1C and FIG. 1D show SEC profile, SDS-page, and WB analysis of rat ADAMTS-7 (rADAMTS-7) (1-575 with respect to SEQ ID NO: 22) from mammalian cell culture. FIG. 1E and FIG. 1F show the domain design of human and rat hybrid ADAMTS-7 (rPro-hCD; with respect to SEQ ID NOs: 22 and 21) with amino acid position indicated below, as well as SEC profile, SDS-page, and WB analysis of purified hybrid protein from mammalian cell culture. Abbreviations: rSP: rat signal peptide; rPro: rat propeptide (the rat prodomain without the signal peptide); hCatalytic: human catalytic domain; hDis: human disintegrin domain. FIGS. 2A, 2B and 2C (consisting of panels A and B) shows the primary structure and sequence comparison of ADAMTS-7. FIG. 2A shows the primary structure of hADAMTS-7 with residue numbers labeled at the domain boundaries (SP: signal peptide; Pro: Prodomain; Dis: Disintegrin domain; TSR: thrombospondin repeat; PLAC: protease and lacunin domain. FIGS. 2B and 2C show a sequence alignment of human and rat ADAMTS-7 in the Pro and CD (catalytic+disintegrin) domains. Different residues were colored in grey, and identical residues were black. Strongly similar residues were black marked with double dots and weakly similar residues were black marked with single dots. Sequence alignment was performed using CLUSTAWL.

FIGS. 3A and 3B (consisting of panels A and B) shows two WT constructs for mammalian expression of ADAMTS-7 proteins. FIG. 3A shows that the SEC profile of hADAMTS-7 CD domain (residues 237-537) reveals production of soluble proteins (fractions B8-B12) are detectible only in the western blot. FIG. 3 B shows that the SEC profile of hADAMTS-7 Pro-CD-TSR1 (residues 1-593) yielded little soluble proteins in the expected elution volumes underlined.

FIGS. 4A and 4B (consisting of panels A and B) shows that furin cleavage site mutants of ADAMTS-7 improved the yield of processed or unprocessed ADAMTS-7. FIG. 4A shows the mutations (grey) introduced into the sequence of the hybrid molecule rPro-hCD to change the furin cleavage efficiency during mammalian cell expression. Q216K mutation in a furin cleavage site and a triple mutant R58A/R61A/R217A were named as rPro-hCD-FM2 (SEQ ID No. 2) and rPro-hCD-3RA respectively. FIG. 4B shows that rPro-hCD-FM2 (SEQ ID No. 2) improved the processing to generate more hCD domains during mammalian production and rPro-hCD-3RA abolished the processing to generate unprocessed protein only. Anti-His WB that targets the 6×His tag at the C-terminus of the protein was used to analyze the raw media of the mammalian cell culture two days post-transfection.

FIG. 5 shows an alignment between mouse ADAMTS-7 prodomain and rat ADAMTS-7 prodomain.

FIG. 6 shows that ADAMTS-12 expression is improved when rat prodomain is used. The first gel was run under non-reducing conditions, whereas the second gel was run under reducing conditions. E393Q substitution (EQ) resulted in increased protein yield. Mutation numbering E393Q follows the species based positions for the human ADAMTS12 region of the construct, i.e. rat ADAMTS12 SP-Pro (1-244) followed by human ADAMTS12 CD (241-544).

FIGS. 7A and 7B show FRET peptide substrate evaluation for TSP-1 and COMP candidate regions.

FIG. 8A-FIG. 8C: The top panels show curves following enzymatic activity over time with different substrates. The lower panels illustrate the rates of activity for rPro-hCD (SEQ ID NO: 1), rPro-hCD-FM2 (SEQ ID NO: 2) and rADAMTS-7(1-575) (SEQ ID NO: 3), respectively, using different substrates. TSP1-1 substrate (SEQ ID NO: 4) is most efficiently turned over by all ADAMTS-7 active constructs.

FIG. 9: Specific activities for the proteolysis of substrate SEQ ID NO: 4-10 show that TSP1-1 substrate is most efficiently turned over by the active ADAMTS-7 constructs. The TSP substrate sequences indicate the cleavage site (underlined) between glutamate and leucine as determined by mass spectrometry. COMP substrate cleavage products were below the detection limit of the mass spectrometer.

### BRIEF DESCRIPTION OF THE SEQUENCE IDS

The sequence listing provided with the application via electronic filing is included herein in its entirety. Where the sequence information or listing apart from the amino acid sequence comprises modifications, fluorophores and/or quencher (i.e. within the feature data) these shall not be read restrictively but only in an exemplary way. The same holds true for non essential modifications such as tags such as FLAG tags or HIS tags.

| SEQ | | |
|---|---|---|
| ID | | |
| NO. | VARIANT | TYPE |
| 1 | rPro-hCD(Rat 1-217/Human 237-537)-TEV-2Strep-6His | PRT |
| | MHRGLNLLLILCALAPHVLGPASGLPTEGRAGLDIVHPVRVDAGGSFLSYELWPRVLRKRDVSAAQASS | |
| | AFYQLQYQGRELLFNLTTNPYLLAPGFVSEIRRRSNLSNVHIQTSVPTCHLLGDVQDPELEGGFAAISA | |
| | CDGLRGVFQLSNEDYFIEPLDEVPAQPGHAQPHMVYKHKRSGQQDDSRTSGTCGVQGSPELKHQREHWE | |
| | QRQQKRRQQRSVSKEKWVETLVVADAKMVEYHGQPQVESYVLTIMNMVAGLFHDPSIGNPIHITIVRLV | |
| | LLEDEEEDLKITHHADNTLKSFCKWQKSINMKGDAHPLHHDTAILLTRKDLCAAMNRPCETLGLSHVAG | |
| | MCQPHRSCSINEDTGLPLAFTVAHELGHSFGIQHDGSGNDCEPVGKRPFIMSPQLLYDAAPLTWSRCSR | |
| | QYITRFLDRGWGLCLDDPPAKDIIDFPSVPPGVLYDVSFIQCRLQYGAYSAFCEDMDNVCHTLWCSVGT | |
| | TCHSKLDAAVDGTRCGENKWCLSGECVPVGFRPEAVGSENLYFQSGWSHPQFEKGGGSGGGSGGGS | |
| | WSHPQFEKHHHHHH | |
| 2 | rPro-hCD-FM2 (Rat 1-217/Human 237-537 FM2 (Q216K))-TEV-2Strep-6His | PRT |
| | MHRGLNLLLILCALAPHVLGPASGLPTEGRAGLDIVHPVRVDAGGSFLSYELWPRVLRKRDVSAAQASS | |
| | AFYQLQYQGRELLFNLTTNPYLLAPGFVSEIRRRSNLSNVHIQTSVPTCHLLGDVQDPELEGGFAAISA | |
| | CDGLRGVFQLSNEDYFIEPLDEVPAQPGHAQPHMVYKHKRSGQQDDSRTSGTCGVQGSPELKHQREHWE | |
| | QRQQKRRQKRSVSKEKWVETLVVADAKMVEYHGQPQVESYVLTIMNMVAGLFHDPSIGNPIHITIVRLV | |
| | LLEDEEEDLKITHHADNTLKSFCKWQKSINMKGDAHPLHHDTAILLTRKDLCAAMNRPCETLGLSHVAG | |
| | MCQPHRSCSINEDTGLPLAFTVAHELGHSFGIQHDGSGNDCEPVGKRPFIMSPQLLYDAAPLTWSRCSR | |
| | QYITRFLDRGWGLCLDDPPAKDIIDFPSVPPGVLYDVSHQCRLQYGAYSAFCEDMDNVCHTLWCSVGTT | |
| | CHSKLDAAVDGTRCGENKWCLSGECVPVGFRPEAVGSENLYFQSGWSHPQFEKGGGSGGGSGGGSW | |
| | SHPQFEKHHHHHH | |
| | rADAMTS-7 (Rat 1-575)-Flag | |
| 3 | MHRGLNLLLILCALAPHVLGPASGLPTEGRAGLDIVHPVRVDAGGSFLSYELWPRVLRKRDVSAAQASS | PRT |
| | AFYQLQYQGRELLFNLTTNPYLLAPGFVSEIRRRSNLSNVHIQTSVPTCHLLGDVQDPELEGGFAAISA | |
| | CDGLRGVFQLSNEDYFIEPLDEVPAQPGHAQPHMVYKHKRSGQQDDSRTSGTCGVQGSPELKHQREHW | |
| | EQRQQKRRQQRSISKEKWVETLVADSKMVEYHGQPQVESYVLTIMNMVAGLYHDPSIGNPIHITWRLI | |
| | ILEDEEKDLKITHHADDTLKNFCRWQKNVNMKGDDHPQHHDTAILLTRKDLCATMNHPCETLGLSHVA | |
| | GLCHPQLSCSVSEDTGLPLAFTVAHELGHSFGIQHDGTGNDCESIGKRPFIMSPQLLYDRGIPLTWSR | |
| | CSREYITRFLDRGWGLCLDDRPSKGVINFPSVLPGVLYDVNHQCRLQYGPSSAYCEDVDNVCYTLWCS | |
| | VGTTCHSKMDAAVDGTSCGKNKWCLNGECVPEGFQPETVDGGWSGWSAWSVCSRSCGVGVRSSERQCT | |
| | QPVPKNKGKYCVGERKRYRLCNLQACPENLYFQGDYKDDDDK | |
| 4 | Peptide Substrate for ADAMTS-7/12 | PRT |
| | (HiLyteFluor-488)-DELSSMVLELRGLRT-K(QXL520)-NH2 | |
| 5 | Peptide Substrate for ADAMTS-7/12 | PRT |
| | (HiLyteFluor-488)-SSMVLELRGLRTIVT-K(QXL520)-NH2 | |
| 6 | Peptide Substrate for ADAMTS-7/12: | |
| | (HiLyteFluor-488)-KVTEENKELANELRR-K(QXL520)-NH2 | PRT |
| 7 | Peptide Substrate for ADAMTS-7/12: | |
| | (HiLyteFluor-488)-EENKELANELRRPPL-K(QXL520)-NH2 | PRT |
| 8 | Peptide Substrate for ADAMTS-7/12: | |
| | (Hi LyteFluor-488)-SSMVLELRG LRT-K(QXL520)-NH2 | PRT |
| 9 | Peptide Substrate for ADAMTS-7/12: | PRT |
| | (Donor) GMQQSVRTGLPS (K-Quencher) | |
| 10 | Peptide Substrate for ADAMTS-7/12: | PRT |
| | (Donor) SPGFRCEACPPGYS (K-Quencher) | |
| 11 | Peptide Substrate for ADAMTS-7 and ADAMTS-12: | PRT |
| | (HiLyteFluor-488)-DELSSMVLELRGLRT-K(QXL520)-E-NH2 | |
| 12 | Peptide Substrate for ADAMTS-7/12: | |
| | (HiLyteFluor-488)-DELSSMVLELRGLRT-K(QXL520)-K-NH2 | PRT |
| 13 | Peptide Substrate for ADAMTS-7/12: | |
| | (HiLyteFluor-488)-DELSSMVLELRGLRT-K(QXL520)-OH | PRT |
| 14 | Peptide Substrate for ADAMTS4 and for ADAMTS5: | PRT |
| | Dabcyl-EEVKAKVQPY-Glu(Edans)-NH2 | |
| 15 | rPro-hCDfor ADAMTS-12 | |
| | MPCAQGNWMAKLSMVAQLLNFGAFCHGRQAQPWPVRFPDPKQEHFIKSLPEYHIVSPVQVDASGHFL | PRT |
| | SYGLHHPVTGSRKKRAAGGSGDQVYYRISHEEKNLFFNLTVNWEFLSNGYWERRYGNLSHVKMAASS | |
| | GQPCHLRGTVLQQGPTIRMGTAALSACQGLTGFFHLPHGDFFIEPVKKHPLTEEGYQPHVIYRRQSY | |
| | RVPETKEPTCGLKDSLDNSVKQELQREKWERKNWPSRSLSRRSISKERWVETLVVADTKMIEYHGSE | |
| | NVESYILTIMNMVTGLFHNPSIGNAIHIVWVRLILLEEEEQGLKIVHHAEKTLSSFCKWQKSINPKS | |
| | DLNPVHHDVAVLLTRKDICAGFNRPCETLGLSHLSGMCQPHRSCNINEDSGLPLAFTIAHELGHSFG | |
| | IQHDGKENDCEPVGRHPYIMSRQLQYDPTPLTWSKCSEEYITRFLDRGWGFCLDDIPKKKGLKSKVI | |
| | APGVIYDVHHQCQLQYGPNATFCQEVENVCQTLWCSVKGFCRSKLDAAADGTQCGEKKWCMAGKCIT | |
| | VGKKPESIPGGGGSDYKDHDGDYKDHDIDYKDDDDK | |
| 16 | ADAMTS Catalytic Motif Consensus Sequence: | PRT |
| | HEXXHXBG(/N/S)BXHD | |
| 17 | Peptide Substrate forMMP12 | PRT |
| | Mca-Pro-Leu-Gly-Leu-Glu-Glu-Ala-Dap(Dnp)-NH2 | |
| 18 | Peptide Substrate forMMP15 | PRT |
| | MCA-Lys-Pro-Leu-Gly-Leu-DPA-Ala-Arg-NH2 | |
| 19 | Peptide Substrate forMMP2 | PRT |
| | MCA-Pro-Leu-Gly-Leu-DPA-Ala-Arg-NH2 | |
| 20 | Peptide Substrate forADAM17 | PRT |
| | Mca-Pro-Leu-Ala-Gln-Ala-Val-Dap(Dnp)-Arg-Ser-Ser-Ser-Arg-NH2 | |
| 21 | Human ADAMTS-7 from Q9UKP4 | |
| | MPGGPSPRSPAPLLRPLLLLLCALAPGAPGPAPGRATEGRAALDIVHPVRVDAGGSFLSY | |
| | ELWPRALRKRDVSVRRDAPAFYELQYRGRELRFNLTANQHLLAPGFVSETRRRGGLGRAH | |
| | IRAHTPACHLLGEVQDPELEGGLAAISACDGLKGVFQLSNEDYFIEPLDSAPARPGHAQP | |
| | HVVYKRQAPERLAQRGDSSAPSTCGVQVYPELESRRERWEQRQQWRRPRLRRLHQRSVSK | |
| | EKWVETLVVADAKMVEYHGQPQVESYVLTIMNMVAGLFHDPSIGNPIHITIVRLVLLEDE | |
| | EEDLKITHHADNTLKSFCKWQKSINMKGDAHPLHHDTAILLTRKDLCAAMNRPCETLGLS | |
| | HVAGMCQPHRSCSINEDTGLPLAFTVAHELGHSFGIQHDGSGNDCEPVGKRPFIMSPQLL | |
| | YDAAPLTWSRCSRQYITRFLDRGWGLCLDDPPAKDIIDFPSVPPGVLYDVSHQCRLQYGA | |
| | YSAFCEDMDNVCHTLWCSVGTTCHSKLDAAVDGTRCGENKWCLSGECVPVGFRPEAVDGG | |
| | WSGWSAWSICSRSCGMGVQSAERQCTQPTPKYKGRYCVGERKRFRLCNLQACPAGRPSFR | |
| | HVQCSHFDAMLYKGQLHTWVPVVNDVNPCELHCRPANEYFAEKLRDAVVDGTPCYQVRAS | |
| | RDLCINGICKNVGCDFEIDSGAMEDRCGVCHGNGSTCHTVSGTFEEAEGLGYVDVGLIPA | |
| | GAREIRIQEVAEAANFLALRSEDPEKYFLNGGWTIQWNGDYQVAGTTFTYARRGNWENLT | |
| | SPGPTKEPVWIQLLFQESNPGVHYEYTIHREAGGHDEVPPPVFSWHYGPWTKCTVTCGRG | |
| | VQRQNVYCLERQAGPVDEEHCDPLGRPDDQQRKCSEQPCPARWWAGEWQLCSSSCGPGGL | |
| | SRRAVLCIRSVGLDEQSALEPPACEHLPRPPTETPCNRHVPCPATWAVGNWSQCSVTCGE | |
| | GTQRRNVLCTNDTGVPCDEAQQPASEVTCSLPLCRWPLGTLGPEGSGSGSSSHELFNEAD | |
| | FIPHHLAPRPSPASSPKPGTMGNAIEEEAPELDLPGPVFVDDFYYDYNFINFHEDLSYGP | |
| | SEEPDLDLAGTGDRTPPPHSHPAAPSTGSPVPATEPPAAKEEGVLGPWSPSPWPSQAGRS | |
| | PPPPSEQTPGNPLINFLPEEDTPIGAPDLGLPSLSWPRVSTDGLQTPATPESQNDFPVGK | |
| | DSQSQLPPPWRDRTNEVFKDDEEPKGRGAPHLPPRPSSTLPPLSPVGSTHSSPSPDVAEL | |
| | WTGGTVAWEPALEGGLGPVDSELWPTVGVASLLPPPIAPLPEMKVRDSSLEPGTPSFPTP | |
| | GPGSWDLQTVAVWGTFLPTTLTGLGHMPEPALNPGPKGQPESLSPEVPLSSRLLSTPAWD | |
| | SPANSHRVPETQPLAPSLAEAGPPADPLVVRNAGWQAGNWSECSTTCGLGAVWRPVRCSS | |
| | GRDEDCAPAGRPQPARRCHLRPCATWHSGNWSKCSRSCGGGSSVRDVQCVDTRDLRPLRP | |
| | FHCQPGPAKPPAHRPCGAQPCLSWYTSSWRECSEACGGGEQQRLVTCPEPGLCEEALRPN | |
| | TTRPCNTHPCTQWVVGPWGQCSGPCGGGVQRRLVKCVNTQTGLPEEDSDQCGHEAWPESS | |
| | RPCGTEDCEPVEPPRCERDRLSFGFCETLRLLGRCQLPTIRTQCCRSCSPPSHGAPSRGH | |
| | QRVARR | |
| 22 | Rat ADAMTS-7 from Q1EHB3 | PRT |
| | MHRGLNLLLILCALAPHVLGPASGLPTEGRAGLDIVHPVRVDAGGSFLSYELWPRVLRKR | |
| | DVSAAQASSAFYQLQYQGRELLFNLTTNPYLLAPGFVSEIRRRSNLSNVHIQTSVPTCHL | |
| | LGDVQDPELEGGFAAISACDGLRGVFQLSNEDYFIEPLDEVPAQPGHAQPHMVYKHKRSG | |
| | QQDDSRTSGTCGVQGSPELKHQREHWEQRQQKRRQQRSISKEKWVETLVVADSKMVEYHG | |
| | QPQVESYVLTIMNMVAGLYHDPSIGNPIHITVVRLIILEDEEKDLKITHHADDTLKNFCR | |
| | WQKNVNMKGDDHPQHHDTAILLTRKDLCATMNHPCETLGLSHVAGLCHPQLSCSVSEDTG | |
| | LPLAFTVAHELGHSFGIQHDGTGNDCESIGKRPFIMSPQLLYDRGIPLTWSRCSREYITR | |
| | FLDRGWGLCLDDRPSKGVINFPSVLPGVLYDVNHQCRLQYGPSSAYCEDVDNVCYTLWCS | |
| | VGTTCHSKMDAAVDGTSCGKNKWCLNGECVPEGFQPETVDGGWSGWSAWSVCSRSCGVGV | |
| | RSSERQCTQPVPKNKGKYCVGERKRYRLCNLQACPPDRPSFRHTQCSQFDSMLYKGKLHK | |
| | WVPVLNDENPCELHCRPFNYSNREKLRDAVMDGTPCYQGRISRDICIDGICKKVGCDFEL | |
| | DSGAEEDRCGVCRGDGSTCHTVSRTFKEAEGMGYVDVGLIPAGAREILIEEVAEAANFLA | |
| | LRSEDPDKYFLNGGWTIQWNGDYQVAGTTFTYTRKGNWETLTSPGPTTEPVWIQLLFQER | |
| | NPGVHYKYTIQRASHSEAQPPEFSWHYGPWSKCPVTCGTGVQRQSLYCMEKQAGIVDEGH | |
| | CDHLSRPRDRKRKCNEEPCPARWWVGDWQPCSRSCGPGGFFRRAVFCTRSVGLDEQRALE | |
| | PSACGHLPRPLAEIPCYHYVACPSSWGVGNWSQCSVTCGAGIRQRSVLCINNTGVPCDGA | |
| | ERPITETFCFLQPCQYSTYIVDTGASGSGSSSPELFNEVDFDPHQPVPRPSPASSPKPVS | |
| | ISNAIDEEDPELDPPGPVFVDDFYYDYNFINFHEDLSYGSFEESHSDLVDIGGQTVPPHI | |
| | RPTEPPSDSPVPTAGAPGAEEEGIQGSWSPSPLLSEASHSPPVLLENTPVNPLANFLTEE | |
| | ESPIGAPELGLPSVSWPPASVDGMVTSVAPGNPDELLVREDTQSQPSTPWSDRNKLSKDG | |
| | NPLGPTSPALPKSPFPTQPSSPSNSTTQASLSPDAVEVSTGWNVALDPVLEADLKPVHAP | |
| | TDPGLLDQIQTPHTEGTQSPGLLPRPAQETQTNSSKDPAVQPLQPSLVEDGAPTDLLPAK | |
| | NASWQVGNWSQCSTTCGLGAIWRLVRCSSGNDEDCTLSSRPQPARHCHLRPCAAWRAGNW | |
| | SKCSRNCGGGSATRDVQCVDTRDLRPLRPFHCQPGPTKPPTRQLCGTQPCLPWYTSSWRE | |
| | CSEACGGGEQQRLVTCPEPGLCEESLRPNNTRPCNTHPCTQWWVGPWGQCSAPCGGGVQR | |
| | RLVKCVNTQTGLAEEDSDLCSHEAWPESSRPCATEDCELVEPSRCERDRLPFNFCETLRL | |
| | LGRCQLPTIRAQCCRSCPPLSRGVPSRGHQRVARR | |
| 23 | Mouse ADAMTS-7 from Q68SA9 | PRT |
| | MHRGPSLLLILCALASRVLGPASGLVTEGRAGLDIVHPVRVDAGGSFLSYELWPRVLRKR | |
| | DVSTTQASSAFYQLQYQGRELLFNLTTNPYLMAPGFVSEIRRHSTLGHAHIQTSVPTCHL | |
| | LGDVQDPELEGGFAAISACDGLRGVFQLSNEDYFIEPLDGVSAQPGHAQPHVVYKHQGSR | |
| | KQAQQGDSRPSGTCGMQVPPDLEQQREHWEQQQQKRRQQRSVSKEKWVETLVVADSKMVE | |
| | YHGQPQVESYVLTIMNMVAGLFHDPSIGNPIHISIVRLIILEDEEKDLKITHHAEETLKN | |
| | FCRWQKNINIKGDDHPQHHDTAILLTRKDLCASMNQPCETLGLSHVSGLCHPQLSCSVSE | |
| | DTGMPLAFTVAHELGHSFGIQHDGTGNDCESIGKRPFIMSPQLLYDRGIPLTWSRCSREY | |
| | ITRFLDRGWGLCLDDRPSKDVIALPSVLPGVLYDVNHQCRLQYGSHSAYCEDMDDVCHTL | |
| | WCSVGTTCHSKLDAAVDGTSCGKNKWCLKGECVPEGFQPEAVDGGWSGWSAWSDCSRSCG | |
| | VGVRSSERQCTQPVPKNRGKYCVGERKRSQLCNLPACPPDRPSFRHTQCSQFDGMLYKGK | |
| | LHKWVPVPNDDNPCELHCRPSNSSNTEKLRDAVVDGTPCYQSRISRDICLNGICKNVGCD | |
| | FVIDSGAEEDRCGVCRGDGSTCQTVSRTFKETEGQGYVDIGLIPAGAREILIEEVAEAAN | |
| | FLALRSEDPDKYFLNGGWTIQWNGDYRVAGTTFTYARKGNWENLTSPGPTSEPVWIQLLF | |
| | QEKNPGVHYQYTIQRDSHDQVRPPEFSWHYGPWSKCTVTCGTGVQRQSLYCMERQAGVVA | |
| | EEYCNTLNRPDERQRKCSEEPCPPRWWAGEWQPCSRSCGPEGLSRRAVFCIRSMGLDEQR | |
| | ALELSACEHLPRPLAETPCNRHVICPSTWGVGNWSQCSVTCGAGIRQRSVLCINNTDVPC | |
| | DEAERPITETFCFLQPCQYPMYIVDTGASGSGSSSPELFNEVDFIPNQLAPRPSPASSPK | |
| | PVSISNAIDEEELDPPGPVFVDDFYYDYNFINFHEDLSYGSFEEPHPDLVDNGGWTAPPH | |
| | IRPTESPSDTPVPTAGALGAEAEDIQGSWSPSPLLSEASYSPPGLEQTSINPLANFLTEE | |
| | DTPMGAPELGFPSLPWPPASVDDMMTPVGPGNPDELLVKEDEQSPPSTPWSDRNKLSTDG | |
| | NPLGHTSPALPQSPIPTQPSPPS1SPTQASPSPDVVEVSTGWNAAWDPVLEADLKPGHGE | |
| | LPSTVEVASPPLLPMATVPGIWGRDSPLEPGTPTFSSPELSSQHLKTLTMPGTLLLTVPT | |
| | DLRSPGPSGQPQTPNLEGTQSPGLLPTPARETQTNSSKDPEVQPLQPSLEEDGDPADPLP | |
| | ARNASWQVGNWSQCSTTCGLGAIWRLVSCSSGNDEDCTLASRPQPARHCHLRPCAAWRTG | |
| | NWSKCSRNCGGGSSTRDVQCVDTRDLRPLRPFHCQPGPTKPPNRQLCGTQPCLPWYTSSW | |
| | RECSEACGGGEQQRLVTCPEPGLCEESLRPNNSRPCNTHPCTQWVVGPWGQCSAPCGGGV | |
| | QRRLVRCVNTQTGLAEEDSDLCSHEAWPESSRPCATEDCELVEPPRCERDRLSFNFCETL | |
| | RLLGRCQLPTIRAQCCRSCPPLSRGVPSRGHQRVARR | |
| 24 | Human ADAMTS-12 from P58397 | PRT |
| | MPCAQRSWLANLSVVAQLLNFGALCYGRQPQPGPVRFPDRRQEHFIKGLPEYHVVGPVRV | |
| | DASGHFLSYGLHYPITSSRRKRDLDGSEDWVYYRISHEEKDLFFNLTVNQGFLSNSYIME | |
| | KRYGNLSHVKMMASSAPLCHLSGTVLQQGTRVGTAALSACHGLTGFFQLPHGDFFIEPVK | |
| | KHPLVEGGYHPHIVYRRQKVPETKEPTCGLKDSVNISQKQELWREKWERHNLPSRSLSRR | |
| | SISKERWVETLVVADTKMIEYHGSENVESYILTIMNMVTGLFHNPSIGNAIHIVVVRLIL | |
| | LEEEEQGLKIVHHAEKTLSSFCKWQKSINPKSDLNPVHHDVAVLLTRKDICAGFNRPCET | |
| | LGLSHLSGMCQPHRSCNINEDSGLPLAFTIAHELGHSFGIQHDGKENDCEPVGRHPYIMS | |
| | RQLQYDPTPLTWSKCSEEYITRFLDRGWGFCLDDIPKKKGLKSKVIAPGVIYDVHHQCQL | |
| | QYGPNATFCQEVENVCQTLWCSVKGFCRSKLDAAADGTQCGEKKWCMAGKCITVGKKPES | |
| | IPGGWGRWSPWSHCSRTCGAGVQSAERLCNNPEPKFGGKYCTGERKRYRLCNVHPCRSEA | |
| | PTFRQMQCSEFDTVPYKNELYHWFPIFNPAHPCELYCRPIDGQFSEKMLDAVIDGTPCFE | |
| | GGNSRNVCINGICKMVGCDYEIDSNATEDRCGVCLGDGSSCQTVRKMFKQKEGSGYVDIG | |
| | LIPKGARDIRVMEIEGAGNFLAIRSEDPEKYYLNGGFIIQWNGNYKLAGTVFQYDRKGDL | |
| | EKLMATGPTNESVWIQLLFQVTNPGIKYEYTIQKDGLDNDVEQQMYFWQYGHWTECSVTC | |
| | GTGIRRQTAHCIKKGRGMVKATFCDPETQPNGRQKKCHEKACPPRWWAGEWEACSATCGP | |
| | HGEKKRTVLCIQTMVSDEQALPPTDCQHLLKPKTLLSCNRDILCPSDWTVGNWSECSVSC | |
| | GGGVRIRSVTCAKNHDEPCDVTRKPNSRALCGLQQCPSSRRVLKPNKGTISNGKNPPTLK | |
| | PVPPPTSRPRMLTTPTGPESMSTSTPAISSPSPTTASKEGDLGGKQWQDSSTQPELSSRY | |
| | LISTGSTSQPILTSQSLSIQPSEENVSSSDTGPTSEGGLVATTTSGSGLSSSRNPITWPV | |
| | TPFYNTLTKGPEMEIHSGSGEEREQPEDKDESNPVIWTKIRVPGNDAPVESTEMPLAPPL | |
| | TPDLSRESWWPPFSTVMEGLLPSQRPTTSETGTPRVEGMVTEKPANTLLPLGGDHQPEPS | |
| | GKTANRNHLKLPNNMNQTKSSEPVLTEEDATSLITEGFLLNASNYKQLTNGHGSAHWIVG | |
| | NWSECSTTCGLGAYWRRVECSTQMDSDCAAIQRPDPAKRCHLRPCAGWKVGNWSKCSRNC | |
| | SGGFKIREIQCVDSRDHRNLRPFHCQFLAGIPPPLSMSCNPEPCEAWQVEPWSQCSRSCG | |
| | GGVQERGVFCPGGLCDWTKRPTSTMSCNEHLCCHWATGNWDLCSTSCGGGFQKRTVQCVP | |
| | SEGNKTEDQDQCLCDHKPRPPEFKKCNQQACKKSADLLCTKDKLSASFCQTLKAMKKCSV | |
| | PTVRAECCFSCPQTHITHTQRQRRQRLLQKSKEL | |
| 25 | Rat ADAMTS-12 from D3ZTJ3 | PRT |
| | MPCAQGNWMAKLSMVAQLLNFGAFCHGRQAQPWPVRFPDPKQEHFIKSLPEYHIVSPVQV | |
| | DASGHFLSYGLHHPVTGSRKKRAAGGSGDQVYYRISHEEKNLFFNLTVNWEFLSNGYVVE | |
| | RRYGNLSHVKMAASSGQPCHLRGTVLQQGPTIRMGTAALSACQGLTGFFHLPHGDFFIEP | |
| | VKKHPLTEEGYQPHVIYRRQSYRVPETKEPTCGLKDSLDNSVKQELQREKWERKNWPSRS | |
| | LSRRSISKERWVETLVVADTKMVEYHGSENVESYILTIMNMVTGLFHNPSIGNAVHIVVV | |
| | RLILLEEEEQGLKIVHHAEKTLSSFCKWQKSINPKSDLNPVHHDVAVLITRKDICAGVNR | |
| | PCETLGLSQLSGMCQPHRSCNINEDSGLPLAFTIAHELGHSFGIQHDGKENDCEPVGRHP | |
| | YIMSQQIQYDPTPLTWSKCSKEYITRFLDRGRGFCLDDVPRKKGLKSNVIAPGVIYDVHH | |
| | QCQLQYGPNATFCQEVENVCQTLWCSVKGFCRSKLDAAADGTRCGEKKWCMAGKCITVGK | |
| | KPESIPGGWGRWSPWSHCSRTCGAGAQSAERLCNNPEPKFGGKYCTGERKRYRLCNVHPC | |
| | RSDTPTFRQMQCSEFDTVPYKNQFYRWFPVFNPAHPCELYCRPIDEQFSERMLEAVIDGT | |
| | PCFEGGNSRNVCINGICKRVGCDYEIDSNATEDRCGVCLGDGSACQTVKKVFRQKEGSGY | |
| | IDIGLIPKGARDIRVMEIKAAGNFLAIRSEDPEKYYLNGGFIIQWNGNYKLAGTVFQYDR | |
| | KGDLERLMAPGPTNESVWLQLLFQVTNPGIKYEYTVRKDGLDNDVEKLLYFWQFGRWTEC | |
| | SVTCGTGIRRQTAHCVKKGHGIVKTTFCNPETQPSVRQKKCYEKDCPPRWWAGEWEACSM | |
| | TCGPYGEKKRTVLCIQTMGSDEQALPATDCQHLLKPKTLVSCNRDILCPSDWTVGNWSEC | |
| | SVSCGGGVRIRSVTCAKNLNEPCDKTRKPNSRALCGLQQCPFSRRVLKPNKDTVPSGKNP | |
| | TTSEHDHFKPIPASTSRPTPLSTPTVPESVSTSTPTINSLGSTITSQEEPDGIGWQNNST | |
| | QAEEDSHIPTSVGSTSQTPLTSWSWSMQPDDENVSSSAIGPTSESDFWATTSDSGLSSSN | |
| | AMTWQVTPFYSTATTEPEVEIHSGSGEDSDQPLNKEENNSVLWNKIRVPERDAPMEMDAE | |
| | IPLGPPPTSYVTEESSWPPFSTMMKSSLPAWSFKNETPRDEGMITEKSGNIPLPLGGDHQ | |
| | TTSPEKLGNNDQLASANSTNPTQGSGPVLTEEDASTLIEEGFLLNASNYKHLMKDHSPAH | |
| | WIVGNWSKCSTTCGLGAYWRSVECSTGMNADCAAIQRPDPAKKCHLRPCAGWRVGNWSKC | |
| | SRNCSGGFKIREVQCMDGVDHHRSLRPFHCQFLAGVPPPLSMSCNLEPCEEWKVEPWSQC | |
| | SRSCGGGVQERGVFCPGGLCDWTKRPASTVPCNRHLCCHWATGNWELCTTSCGGGSQKRT | |
| | VHCIPSENSTTEDQDQCFCDHQARPPEFQNCNQQACRKSADLTCTKDRLSTSFCQTLKSM | |
| | KKCSVPSVRVQCCLSCPQTQSIHTQRQRKQQMLQNHDTL | |
| 26 | Mouse ADAMTS-12 from Q811B3 | PRT |
| | MPCARGSWLAKLSIVAQLINFGAFCHGRQTQPWPVRFPDPRQEHFIKSLPEYHIVSPVQV | |
| | DAGGHVLSYGLHHPVTSSRKKRAAGGSGDQLYYRISHEEKDLFFNLTVNWEFLSNGYVVE | |
| | KRYGNLSHVKMVASSGQPCHLRGTVLQQGTTVGIGTAALSACQGLTGFFHLPHGDFFIEP | |
| | VKKHPLTEEGSYPHVVYRRQSIRAPETKEPICGLKDSLDNSVKQELQREKWERKTLRSRS | |
| | LSRRSISKERWVETLVVADTKTVEYHGSENVESYILTIMNMVTGLFHSPSIGNLVHIVVV | |
| | RLILLEEEEQGLKIVHHAEKTLSSFCKWQKSINPKSDLNPVHHDVAVLITRKDICAGVNR | |
| | PCETLGLSQLSGMCQPHRSCNINEDSGLPLAFTIAHELGHSFGIQHDGKENDCEPVGRHP | |
| | YIMSQQIQYDPTPLTWSKCSKEYITRFLDRGRGFCLDDIPSKKGLKSNVIAPGVIYDVHH | |
| | QCQLQYGPNATFCQEVENVCQTLWCSVKGFCRSKLDAAADGTRCGEKKWCMAGKCITVGK | |
| | KPESIPGGWGRWSPWSHCSRTCGAGAQSAERLCNNPEPKFGGKYCTGERKRYRLCNVHPC | |
| | RSDTPTFRQMQCSEFDTVPYKNQFYRWFPVFNAAHPCELYCRPIDEQFSERMLEAVIDGT | |
| | PCFEGGNSRNVCINGICKRVGCDYEIDSNATEDRCGVCLGDGSACQTVKKLFRQKEGSGY | |
| | VDIGLIPKGARDIRVMEIKAAGNFLAIRSEDPEKYYLNGGFIIQWNGNYKLAGTVFQYDR | |
| | KGDLEKLIAPGPTNESVWLQLLFQVTNPGIKYEYTVRKDGLDNDVEKLLYFWQFGRWTEC | |
| | SVTCGTGIRRQAAHCVKKGHGIVKTTFCNPETQPSVRQKKCHEKDCPPRWWAGEWEACST | |
| | TCGPYGEKKRTVLCIQTMGSDEQALPATDCQHLLKPKALVSCNRDILCPSDWTVGNWSEC | |
| | SVSCGGGVRIRSVTCAKNLNEPCDKTRKPNSRALCGLQQCPFSRRVLKPNKDIAPSGKNQ | |
| | STAEHDPFKPIPAPTSRPTPLSTPTVPESMSTSTPTINSLGSTIASQEDANGMGWQNNST | |
| | QAEEGSHFPTSSGSTSQVPVTSWSLSIQPDDENVSSSAIGPTSEGDFWATTTSDSGLSSS | |
| | DAMTWQVTPFYSTMTTDPEVEIHSGSGEDSDQPLNKDKSNSVIWNKIGVPEHDAPMETDA | |
| | ELPLGPPPTSYMGEEPSWPPFSTKMEGSLPAWSFKNETPRDDGMIAEKSRKIPLPLAGDH | |
| | HPATSEKLENHDKLALPNTTNPTQGFGPVLTEEDASNLIAEGFLLNASDYKHLMKDHSPA | |
| | YWIVGNWSKCSTTCGLGAYWRSVECSSGVDADCTTIQRPDPAKKCHLRPCAGWRVGNWSK | |
| | CSRNCSGGFKIREVQCMDSLDHHRSLRPFHCQFLAGAPPPLSMSCNLEPCGEWQVEPWSQ | |
| | CSRSCGGGVQERGVSCPGGLCDWTKRPATTVPCNRHLCCHWATGNWELCNTSCGGGSQKR | |
| | TIHCIPSENSTTEDQDQCLCDHQVKPPEFQTCNQQACRKSADLTCLKDRLSISFCQTLKS | |
| | MRKCSVPSVRAQCCLSCPQAPSIHTQRQRKQQLLQNHDML | |

According to a first aspect the present invention provides novel low-molecular-weight compounds which act as potent antagonists of the ADAMTS7 receptor and are thus suitable for treatment and/or prevention of lung diseases, heart diseases, inflammatory diseases, fibrotic diseases, metabolic diseases, cardiometabolic diseases, vascular diseases and/or cardiovascular diseases.

Further embodiments are directed to the identification of ADAMTS7 antagonists that are suitable for treatment and/or prevention of atherosclerosis, athersclerosis-related diseases such as coronary artery disease or peripheral vascular disease/arterial occlusive disease as well as post-surgery complications of these diseases such as restenosis after angioplasty.

Further embodiments are directed to the identification of selective ADAMTS7 antagonists in respect to ADAMTS4 antagonistic effect.

WO 2014/06651 discloses substituted hydantoinamides as ADAMTS4 and 5 inhibitors for use in the treatment of arthritis in particular osteoarthritis.

WO 2004/024721 and WO2004024715 describe hydantoin derivatives and their use as TACE (ADAMT17) inhibitors.

WO2004/108086 and WO2002/096426 disclose Hydantoin derivatives as TACE inhibitors.

WO 2017/211666 and WO2017/211667 disclose Hydantoin derivatives as ADAMTS4 and 5 inhibitors for the treatment of inflammatory diseases preferably osteoarthritis.

WO01/44200 generically discloses a broad range of compounds as selective neurokinin antagonists.

WO2018/069532 discloses inhibitors of alpha-amino-beta carboxymuconic acid semialdehyde decarboxylase.

9 distinct compounds with the following CAS numbers 2224459-87-2, 2224418-23-7, 2224397-97-9, 2224363-23-7, 2224363-22-6, 2224237-51-6, 2224237-28-7, 2224196-43-2 and 2224145-02-0 have been published without further information with regard to any pharmaceutical use.

It has now been found that compounds of the present invention have surprising and advantageous properties.

In particular, it has been found that compounds of the present invention are ADAMTS7 antagonists, many of them potent and selective antagonists for ADAMTS7. Moreover, many of the compounds of the invention are selective with respect to ADAMTS4—preferably 10 times, or even 50 times more selective against ADAMTS7 relative to ADAMTS4.

The present invention provides compounds of general formula (I):

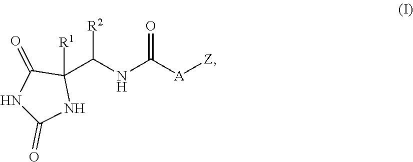

in which
R¹ represents a group selected from hydrogen, (C₁-C₆)-alkyl, (C₃-C₆)-cycloalkyl, 5- to 6-membered heterocyloalkyl, 5- to 6-membered heteroaryl and phenyl,
   wherein said (C₁-C₆)-alkyl, (C₃-C₆)-cycloalkyl, 5- to 6-membered heterocycloalkyl, and 5- to 6-membered heteroaryl, phenyl are optionally substituted with one or two groups independently selected from cyano, halogen, amino, hydroxy, oxo, (C₁-C₃)-alkyl, (C₁-C₄)-alkoxy, (C₁-C₄)-alkylcarbonyl, mono-(C₁-C₄)-alkylamino, di-(C₁-C₄)-alkylamino, (C₁-C₄)-alkylsulfonyl, (C₃-C₆)-cycloalkyl,
   wherein each said (C₁-C₃)-alkyl, (C₃-C₆)-cycloalkyl, and (C₁-C₄)-alkoxy is optionally substituted with up to 5 fluorine atoms,
R² represents a group selected from hydrogen or (C₁-C₄)-alkyl
   wherein said (C₁-C₄)-alkyl is optionally substituted with up to five fluorine atoms,
A represents a group selected from 5-membered heteroaryl
   wherein said 5-membered heteroaryl is optionally substituted with one, two or three groups independently selected from halogen, cyano, hydroxy, methylsulfonyl, (C₃-C₆)-cycloalkyl, (C₁-C₄)-alkyl, and (C₁-C₄)-alkoxy,
   wherein each said (C₃-C₆)-cycloalkyl, (C₁-C₄)-alkyl and (C₁-C₄)-alkoxy is optionally substituted with up to three fluorine atoms,
Z represents a group selected from 6- to 10-membered aryl and 5- to 10-membered heteroaryl,
   wherein said 6- to 10-membered aryl or 5- to 10-membered heteroaryl is optionally substituted with one, two or three groups independently selected from halogen, cyano, hydroxy, (C₁-C₄)-alkylsulfonyl, (C₃-C₆)-cycloalkyl, (C₁-C₄)-alkyl, and (C₁-C₄)-alkoxy,
      wherein each said (C₁-C₄)-alkyl and (C₁-C₄)-alkoxy is optionally substituted with (C₃-C₆)-cycloalkyl and optionally substituted with up to five fluorine atoms,
         and pharmaceutically acceptable salts thereof, solvates thereof and the solvates of the salts thereof.

In certain embodiments, the present invention provides compounds of general formula (I) with the proviso that the following compounds (Xa) to (Xi) are excluded:
4-(4-chlorophenyl)-N-[[4-(1-methylimidazol-2-yl)-2,5-dioxo-imidazolidin-4-yl]methyl]-1H-pyrrole-2-carboxamide

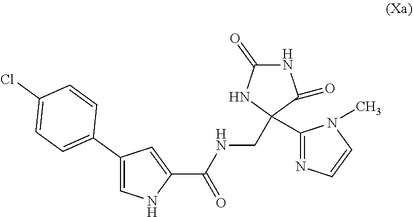 [SMILES: Cn1ccnc1C1(CNC(=O)c2cc(-c3ccc(Cl)cc3)c[nH]2)NC(=O)NC1=O]

1-(4-fluorophenyl)-N-[[4-(1-methylimidazol-2-yl)-2,5-dioxo-imidazolidin-4-yl]methyl]pyrazole-4-carboxamide

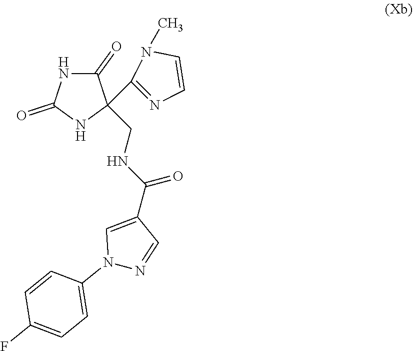 [SMILES: Cn1ccnc1C1(CNC(=O)c2cnn(-c3ccc(F)cc3)c2)NC(=O)NC1=O]

1-methyl-N-[[4-(1-methylimidazol-2-yl)-2,5-dioxo-imidazolidin-4-yl]methyl]-5-phenyl-pyrrole-2-carboxamide

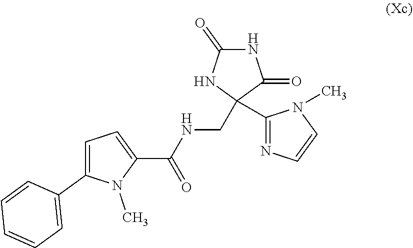 [SMILES: Cn1ccnc1C1(CNC(=O)c2ccc(-c3ccccc3)n2C)NC(=O)NC1=O]

4-(5-chloro-2-thienyl)-N-[[4-(1-methylimidazol-2-yl)-2,5-dioxo-imidazolidin-4-yl]methyl]-1H-pyrrole-3-carboxamide

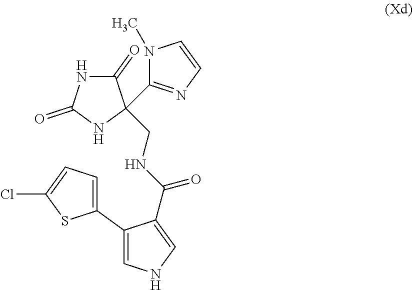 [SMILES: Cn1ccnc1C1(CNC(=O)c2c[nH]cc2-c2ccc(Cl)s2)NC(=O)NC1=O]

4-methyl-N-[[4-(1-methylimidazol-2-yl)-2,5-dioxo-imidazolidin-4-yl]methyl]-2-(2-thienyl)thiazole-5-carboxamide

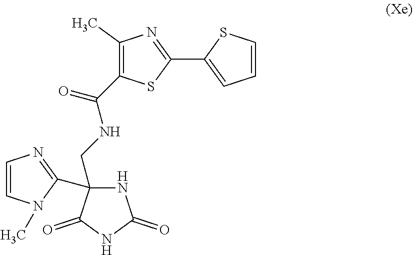 [SMILES: Cc1nc(-c2cccs2)sc1C(=O)NCC1(c2nccn2C)NC(=O)NC1=O]

N-[[4-(1-methylimidazol-2-yl)-2,5-dioxo-imidazolidin-4-yl]methyl]-2-(4-pyridyl)thiazole-5-carboxamide

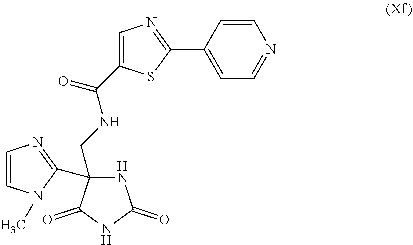 [SMILES: Cn1ccnc1C1(CNC(=O)c2cnc(-c3ccncc3)s2)NC(=O)NC1=O]

5-methyl-N-[[4-(1-methylimidazol-2-yl)-2,5-dioxo-imidazolidin-4-yl]methyl]-1-(4-pyridyl)pyrazole-4-carboxamide

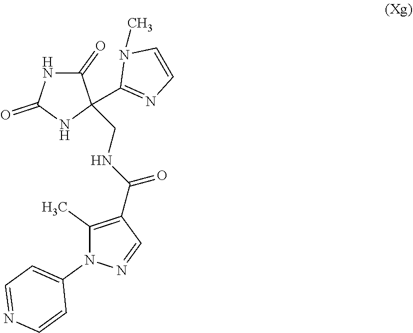 [SMILES: Cc1c(C(=O)NCC2(c3nccn3C)NC(=O)NC2=O)cnn1-c1ccncc1]

N-[[4-(1-methylimidazol-2-yl)-2,5-dioxo-imidazolidin-4-yl]methyl]-5-(1H-pyrazol-3-yl)thiophene-2-carboxamide

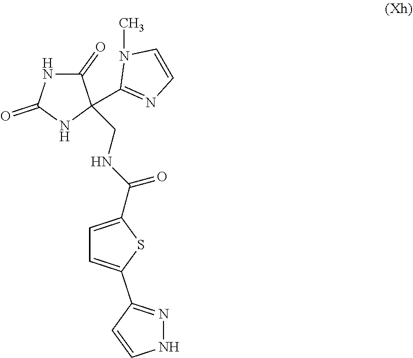 [SMILES: Cn1ccnc1C1(CNC(=O)c2ccc(-c3cc[nH]n3)s2)NC(=O)NC1=O]

N-[[4-(1-methylimidazol-2-yl)-2,5-dioxo-imidazolidin-4-yl]methyl]-5-phenyl-1H-pyrazole-3-carboxamide

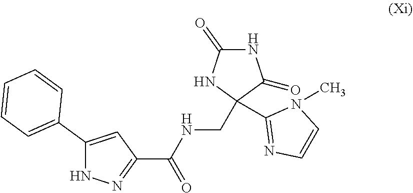 [SMILES: Cn1ccnc1C1(CNC(=O)c2cc(-c3ccccc3)[nH]n2)NC(=O)NC1=O]

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of non-hydrogen substituents can be equal to or different from zero. Unless otherwise indicated, optionally substituted groups may be substituted with as many optional substituents as can be accommodated by replacing a hydrogen atom with a non-hydrogen substituent on any available carbon atom or heteroatom.

When groups in the compounds according to the invention are substituted, said groups may be mono-substituted or poly-substituted with substituent(s), unless otherwise specified. Within the scope of the present invention, the meanings of all groups which occur repeatedly are independent from one another. Groups in the compounds according to the invention may, for example, be substituted with one, two or three identical or different substituents.

As used herein, an “oxo” substituent represents an oxygen atom, which is bound to a carbon atom or to a sulfur atom via a double bond.

As used herein “allyl” is a substituent with the structural formula H₂C═CH—CH₂*, where * is the connection to the rest of the molecule. It consists of a methylene bridge (—CH₂—) attached to a vinyl group (—CH═CH₂).

The term “ring substituent” means a non-hydrogen substituent attached to an aromatic or nonaromatic ring which replaces an available hydrogen atom on the ring.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen” or “halogen atom” means a fluorine, chlorine, bromine or iodine atom, preferably a fluorine, chlorine or bromine atom, even more preferably fluorine or chlorine most preferred fluorine.

The term “C₁-C₃-alkyl”, “C₁-C₄-alkyl” and “C₁-C₆-alkyl” means a linear or branched, saturated, monovalent hydrocarbon group having 1, 2 or 3, 1, 2, 3, or 4 carbon atoms, and 1, 2, 3, 4, 5 or 6 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Preferably, said group has 1, 2, 3 or 4 carbon atoms (“C₁-C₄-alkyl”), e.g., a methyl, ethyl, propyl, isopropyl, butyl, sec-butyl isobutyl, or tert-butyl group, more preferably 1, 2 or 3 carbon atoms (“C₁-C₃-alkyl”), e.g., a methyl, ethyl, n-propyl or isopropyl group.

The terms “C₁-C₄-alkoxy” means a linear or branched, saturated, monovalent group of formula (C₁-C₄-alkyl)-O—, in which the term “C₁-C₄-alkyl” is as defined supra, e.g., a methoxy, ethoxy, n-propoxy, isopropoxy, butoxy or an isomer thereof, preferably a methoxy-group or ethoxy-group.

The term “C₃-C₆-cycloalkyl” means a saturated, monovalent, mono- or bicyclic hydrocarbon ring which contains 3, 4, 5 or 6 carbon atoms, respectively. Said C₃-C₆-cycloalkyl group may be for example, a monocyclic hydrocarbon ring, e.g., a cyclopropyl, cyclobutyl, cyclopentyl or cyclohexyl group, or a bicyclic hydrocarbon ring. The term “3- to 6-membered cycloalkyl” is equivalent to a “C₃-C₆-cycloalkyl”. Thus a “4-membered cycloalkyl group” has the same meaning as a “C₄-cycloalkyl group”, and a “C₃-cycloalkyl” is a cyclopropyl-group.

The term “5- to 6-membered heterocycloalkyl means a monocyclic, saturated heterocycloalkyl with 5 or 6 ring atoms in total, respectively, which contains one or two identical or different ring heteroatoms from the series N, S or O. The heterocycloalkyl group may be attached to the rest of the molecule via any one of the carbon or nitrogen atoms. A heterocycloalkyl group which contains at least one ring nitrogen atom may be referred to as aza-heterocycloalkyl, and a heterocycloalkyl group which contains at least one ring oxygen atom may be referred to as oxa-heterocycloalkyl. Preferably, an aza-heterocycloalkyl group contains only nitrogen and carbon atoms in the ring, and an oxa-heterocycloalkyl group contains only ring oxygen and carbon atoms in the ring.

Said heterocycloalkyl without being limited thereto, can be a 5-membered ring, such as tetrahydrofuranyl, 1,3-dioxolanyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, for example; or a 6-membered ring, such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, for example.

The term 4- to 5-membered aza-heterocycloalkyl means a monocyclic saturated or unsaturated heterocycloalkyl group with 4 or 5 ring atoms in total, respectively, which contains at least one ring nitrogen atom, and which is attached to the rest of the molecule via a ring carbon atom or optionally via a ring nitrogen atom (if allowed by valency). The following heterocycloalkyls may be mentioned by way of example: Pyrrolidin, Pyrrolin, Azetidin, Oxazolidin

The term “6- to 10-membered aryl” means a mono- or optionally bicyclic aromatic ring with 6 to 10 (preferably 6) carbon ring atoms in total, such as phenyl or naphthyl.

The term “5- to 10-membered heteroaryl”, “5- to 6-membered heteroaryl”, “5-membered heteroaryl” and “6-membered heteroaryl” means a mono- or optionally bicyclic aromatic ring with 5 to 10, 5 to 6, 5, or 6 ring atoms in total, respectively, which contains at least one ring heteroatom and optionally one, two or three furtherring heteroatoms from the series: N, O and/or S, and which is attached to the rest of the molecule via a ring carbon atom or optionally via a ring nitrogen atom (if allowed by valency).

The following representative heteroaryls may be mentioned by way of example: furyl, pyrrolyl, thienyl, pyrazolyl, imidazolyl, thiazolyl, oxazolyl, isoxazolyl, isothiazolyl, triazolyl, oxadiazolyl, thiadiazolyl, pyridyl, pyrimidinyl, pyridazinyl, pyrazinyl, triazinyl, benzofuranyl, benzothienyl, benzimidazolyl, benzoxazolyl, benzothiazolyl, benzotriazolyl, indolyl, indazolyl, quinolinyl, isoquinolinyl, naphthyridinyl, quinazolinyl, quinoxalinyl, phthalazinyl, and pyrazolo[3,4-b]pyridinyl.

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl; or a tricyclic heteroaryl group, such as, for example, carbazolyl, acridinyl or phenazinyl.

“5-membered aza-heteroaryl” in the context of the invention means an aromatic heterocyclic group (heteroaromatic) having 5 ring atoms in total, which contains at least one ring nitrogen atom and optionally one or two further ring heteroatoms selected from N, O and S, and which is bound via a ring carbon atom or optionally via a ring nitrogen atom (if allowed by valency), in particular a 5-membered aza-heteroaryl containing one ring nitrogen atom and one or two further ring heteroatoms selected from N and O, such as: pyrrolyl, pyrazolyl, imidazolyl, oxazolyl, thiazolyl, isoxazolyl, isothiazolyl, triazolyl, oxadiazolyl, and thiadiazolyl, for example, preferred 5-membered aza-heteroaryls being pyrazolyl, imidazolyl, oxazolyl, thiazolyl, triazolyl, isoxazolyl, and oxadiazolyl. Particularly preferred 5-membered aza-heteroarylss are triazolyl, oxazolyl, isoxazolyl, pyrazolyl, thiazolyl, 1,2,4-oxadiazolyl and 1,3,4-oxadiazolyl.

mono-(C₁-C₄)-alkylamino in the context of the invention means an amino group with one straight-chain or branched alkyl substituent which contains 1, 2, 3 or 4 carbon atoms, such as: methyl-amino, ethylamino, n-propylamino, isopropylamino, n-butylamino, and tert-butylamino, for example.

di-(C₁-C₄)-alkylamino in the context of the invention means an amino group with two identical or different straight-chain or branched alkyl substituents which each contain 1, 2, 3 or 4 carbon atoms, such as: N,N-dimethylamino, N,N-diethylamino, N-ethyl-N-methylamino, N-methyl-N-n-propylamino, N-isopropyl-N-methylamino, N-isopropyl-N-n-propylamino, N,N-diisopropylamino, N-n-butyl-N-methylamino, and N-tert-butyl-N-methylamino, for example.

(C₁-C₄)-Alkylcarbonyl in the context of the invention means a straight-chain or branched alkyl group having 1, 2, 3 or 4 carbon atoms which is bound to the rest of the molecule via a carbonyl group [—C(═O)—], such as: acetyl, propionyl, n-butyryl, isobutyryl, n-pentanoyl, and pivaloyl, for example.

(C₁-C₄)-alkylsulfonyl in the context of the invention means a group which is bound to the rest of the molecule via a sulfonyl group [—S(═O)₂-] and which has one straight-chain or branched alkyl substituent having 1, 2, 3 or 4 carbon atoms, such as: methylsulfonyl, ethylsulfonyl, n-propylsulfonyl, isopropylsulfonyl, n-butylsulfonyl, and tert-butylsulfonyl, for example.

An oxo substituent in the context of the invention means an oxygen atom which is bound to a carbon atom via a double bond.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

The term “C₁-C₄”, as used in the present text, e.g., in the context of the definition of “C₁-C₄-alkyl”, “C₁-C₄-alkoxy”, “or “C₁-C₄-alkylsulfanyl”, means an alkyl group having 1 to 4 carbon atoms, i.e., 1, 2, 3, or 4 carbon atoms.

The term “C₁-C₆”, as used in the present text, e.g., in the context of the definition of “C₁-C₆-alkyl”, means an alkyl group having 1 to 6 carbon atoms, i.e., 1, 2, 3, 4, 5 or 6 carbon atoms.

Further, as used herein, the term “C₃-C₆”, as used in the present text, e.g., in the context of the definition of “C₃-C₆-cycloalkyl”, means a cycloalkyl group having 3 to 6 carbon atoms, i.e., 3, 4, 5 or 6 carbon atoms.

When a range of values is given, said range encompasses each value and sub-range within said range.

For example:

“C₁-C₄” encompasses C₁, C₂, C₃, C₄, C₁-C₄, C₁-C₃, C₁-C₂, C₂-C₄, C₂-C₃, and C₃-C₄;

“C₁-C₃” encompasses C₁, C₂, C₃, C₁-C₃, C₁-C₂, and C₂-C₃;

“C₂-C₄” encompasses C₂, C₃, C₄, C₂-C₄, C₂-C₃, and C₃-C₄;

“C₃-C₆” encompasses C₃, C₄, C₅, C₆, C₃-C₆, C₃-C₅, C₃-C₄, C₄-C₆, C₄-C₅, and C₅-C₆;

As used herein, the term “leaving group” means an atom or a group of atoms that is displaced in a chemical reaction as stable species taking with it the bonding electrons. Preferred such leaving groups include halide (e.g., fluoride, chloride, bromide or iodide), and sulfonate (e.g., (methylsulfonyl)oxy (mesyl(ate), Ms), [(trifluoromethyl)sulfonyl]oxy (triflyl/(ate), Tf), [(nonafluoro-butyl)sulfonyl]oxy (nonaflate, Nf), (phenylsulfonyl)oxy, [(4-methylphenyl)sulfonyl]oxy, [(4-bromo-phenyl)sulfonyl]oxy, [(4-nitrophenyl)sulfonyl]oxy, [(2-nitrophenyl)sulfonyl]oxy, [(4-isopropyl-phenyl)sulfonyl]oxy, [(2,4,6-triisopropylphenyl)sulfonyl]oxy, [(2,4,6-trimethylphenyl)sulfonyl]oxy, [(4-tert-butylphenyl)sulfonyl]oxy and [(4-methoxyphenyl)sulfonyl]oxy).

It is possible for the compounds of general formula (I) to exist as isotopic variants. The invention therefore includes one or more isotopic variant(s) of the compounds of general formula (I), particularly deuterium-containing compounds of general formula (I).

The term “Isotopic variant” of a compound or a reagent is defined as a compound exhibiting an unnatural proportion of one or more of the isotopes of the atoms that constitute such a compound.

The term “Isotopic variant of the compound of general formula (I)” is defined as a compound of general formula (I) exhibiting an unnatural proportion of one or more of the isotopes of the atoms that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998.

Examples of such isotopes include stable and radioactive isotopes of hydrogen, carbon, nitrogen, oxygen, phosphorus, sulfur, fluorine, chlorine, bromine and iodine, such as ²H (deuterium), ³H (tritium), ¹¹C ¹³C ¹⁴C ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁵I, ¹²⁹I and ¹³¹I, respectively.

With respect to the treatment and/or prophylaxis of the disorders specified herein, isotopic variant(s) of the compounds of general formula (I) preferably contain elevated levels of deuterium (“deuterium-containing compounds of general formula (I)”). Isotopic variants of the compounds of general formula (I) in which one or more radioactive isotopes, such as ³H or ¹⁴C, are incorporated are useful, e.g., in drug and/or substrate tissue distribution studies. These isotopes are particularly preferred for the ease of their incorporation and detectability. Positron-emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, e.g., by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds (Esaki et al., Tetrahedron, 2006, 62, 10954; Esaki et al., Chem. Eur. J., 2007, 13, 4052). Deuterium gas is also a useful reagent for incorporating deuterium into molecules. Catalytic deuteration of olefinic bonds (H. J. Leis et al Curr. Org. Chem., 1998, 2, 131; J. R. Morandi et al., J. Org. Chem., 1969, 34 (6), 1889) and acetylenic bonds (N. H. Khan, J. Am. Chem. Soc., 1952, 74 (12), 3018; S. Chandrasekhar et al., Tetrahedron Letters, 2011, 52, 3865) is a rapid route for incorporation of deuterium. Metal catalysts (e.g., Pd, Pt, and Rh) in the presence of deuterium gas can be used to directly exchange deuterium for hydrogen in functional groups containing hydrocarbons (J. G. Atkinson et al., U.S. Pat. No. 3,966,781). A variety of deuterated reagents and synthetic building blocks are commercially available from companies such as for example C/D/N Isotopes, Quebec, Canada; Cambridge Isotope Laboratories Inc., Andover, Mass., USA; and CombiPhos Catalysts, Inc., Princeton, N.J., USA. Further information on the state of the art with respect to deuterium-hydrogen exchange is given for example in Hanzlik et al., J. Org. Chem. 55, 3992-3997, 1990; R. P. Hanzlik et al., Biochem. Biophys. Res. Commun. 160, 844, 1989; P. J. Reider et al., J. Org. Chem. 52, 3326-3334, 1987; M. Jarman et al., Carcinogenesis 16(4), 683-688, 1995; J. Atzrodt et al., Angew. Chem., Int. Ed. 2007, 46, 7744; K. Matoishi et al., Chem. Commun. 2000, 1519-1520; K. Kassahun et al., WO2012/112363.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of genera formula (I) may alter the physicochemical properties (such as for example acidity [C. L. Perrin, et al., J. Am. Chem. Soc., 2007, 129, 4490; A. Streitwieser et al., J. Am. Chem. Soc., 1963, 85, 2759;], basicity [C. L. Perrin et al., J. Am. Chem. Soc., 2005, 127, 9641; C. L. Perrin, et al., J. Am. Chem. Soc., 2003, 125, 15008; C. L. Perrin in Advances in Physical Organic Chemistry, 44, 144], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102; D. J. Kushner et al., Can. J. Physiol. Pharmacol., 1999, 77, 79). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and/or enhances the formation of a desired metabolite (e.g., Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels, i.e., reduced peak-trough variation. This could result in lower side effects and/or enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples of this deuterium effect. Still other cases have been reported in which reduced rates of metabolism result in an increase in exposure of the drug without changing the rate of systemic clearance (e.g., Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g., lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I) which are sites of attack for metabolizing enzymes such as, e.g., cytochrome P₄₅₀.

In certain embodiments, the present invention concerns a deuterium-containing compound of general formula (I) having 1, 2, 3 or 4 deuterium atoms (i.e., 1, 2, 3, or 4 sites where the isotopic balance of hydrogen is enriched for deuterium), preferably with 1, 2 or 3 deuterium atoms.

Where the plural form of the word compounds, salts, polymorphs, hydrates, solvates and the like, is used herein, this is taken to include also a single compound, salt, polymorph, isomer, hydrate, solvate or the like.

By “stable compound” or “stable structure” is meant a compound that is sufficiently robust to survive isolation to a useful degree of purity from a reaction mixture, and is capable of being subjected to further chemical transformation or, preferably, formulation into an efficacious therapeutic agent.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds (i.e., atropisomers).

Preferred compounds are those which produce the more desirable biological activity. Separated, pure or partially purified isomers and stereoisomers or racemic or diastereomeric mixtures of the compounds of the present invention are also included within the scope of the present invention.

The purification and the separation of such materials can be accomplished by standard techniques known in the art.

Separated optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials or optically active catalysts.

In order to distinguish different types of isomers from each other, reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g., (R)- or (S)-isomers, in any ratio. Isolation of a single stereoisomer, e.g., a single enantiomer or a single diastereomer, of a compound of the present invention is achieved by any suitable state of the art method, such as chromatography, especially chiral chromatography, for example.

Further, it is possible for the compounds of the present invention to exist as tautomers. For example, a compound of the present invention which contains an imidazopyridine moiety as a heteroaryl group for example can exist as a 1H tautomer, or a 3H tautomer, or even a mixture in any amount of the two tautomers, namely:

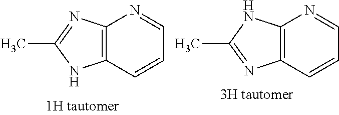

Moreover, in the course of the synthesis of a 1H-pyrazole group, the 1H-pyrazol-3-yl tautomer as well as the 1H-pyrazol-5-yl tautomer are formed.

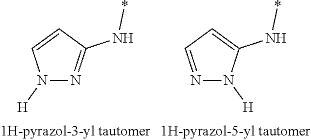 [SMILES: *Nc1cc[nH]n1 ; *Nc1ccn[nH]1]

The present invention includes all possible tautomers of the compounds of the present invention as single tautomers, or as any mixture of said tautomers, in any ratio.

The present invention also covers useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, preferably water, methanol or ethanol, for example, as a structural element of the crystal lattice of the compounds. It is possible for the amount of polar solvents, in particular water, to exist in a stoichiometric or non-stoichiometric ratio. In the case of stoichiometric solvates, e.g., a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g., as a free base, or as a free acid, or as a zwitterion, or to exist in the form of a salt, preferably as a free acid. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, 3-phenylpropionic, pivalic, 2-hydroxyethanesulfonic, itaconic, trifluoromethanesulfonic, dodecylsulfuric, ethanesulfonic, benzenesulfonic, para-toluenesulfonic, methanesulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium, magnesium or strontium salt, or an aluminium or a zinc salt, or an ammonium salt derived from ammonia or from an organic primary, secondary or tertiary amine having 1 to 20 carbon atoms, such as ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol, or a salt with a quaternary ammonium ion having 1 to 20 carbon atoms, such as tetramethylammonium, tetraethylammonium, tetra(n-propyl)ammonium, tetra(n-butyl)ammonium, N-benzyl-N,N,N-trimethylammonium, choline or benzalkonium.

In accordance with preferred embodiments of the first aspect, the present invention covers a pharmaceutically acceptable salt of compounds of general formula (I), supra, which is an alkali metal salt, in particular a sodium or potassium salt, or an ammonium salt derived from an organic tertiary amine, in particular choline.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

As used herein, the term “in vivo hydrolysable ester” means an in vivo hydrolysable ester of a compound of the present invention containing a carboxy or hydroxy group, for example, a pharmaceutically acceptable ester which is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₆ alkoxymethyl esters, e.g., methoxymethyl, C₁-C₆ alkanoyloxymethyl esters, e.g., pivaloyloxymethyl, phthalidyl esters, C₃-C₈ cycloalkoxy-carbonyloxy-C₁-C₆ alkyl esters, e.g. 1-cyclohexylcarbonyloxyethyl; 1,3-dioxolen-2-onylmethyl esters, e.g., 5-methyl-1,3-dioxolen-2-onylmethyl, and C₁-C₆-alkoxycarbonyloxyethyl esters, e.g., 1-methoxycarbonyloxyethyl, it being possible for said esters to be formed at any carboxy group in the compounds of the present invention.

An in vivo hydrolysable ester of a compound of the present invention containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of the compounds of the present invention, either as single polymorph, or as a mixture of more than one polymorph, in any ratio.

Moreover, the present invention also includes prodrugs of the compounds according to the invention. The term “prodrugs” here designates compounds which themselves can be biologically active or inactive, but are converted (for example metabolically or hydrolytically) into compounds according to the invention during their residence time in the body. Typical examples of prodrugs would be e.g. esters.

Preference is given to compounds of formula (I) in which
R¹ represents a group selected from hydrogen, (C₁-C₆-alkyl, (C₃-C₆)-cycloalkyl, 5- to 6-membered heterocycloalykl, 5- to 6-membered heteroaryl and phenyl,
   wherein said (C₁-C₆-alkyl, (C₃-C₆)-cycloalkyl, 5- to 6-membered heterocycloalkyl, 5- to 6-membered heteroaryl and phenyl are optionally substituted with one or two groups independently selected from cyano, halogen, amino, hydroxy, oxo, (C₁-C₃)-alkyl, (C₁-C₄)-alkoxy, (C₁-C₄)-alkylcarbonyl, mono-(C₁-C₄)-alkylamino, di-(C₁-C₄)-alkylamino, (C₁-C₄)-alkylsulfonyl, (C₃-C₆)-cycloalkyl,
   wherein each said (C₁-C₃)-alkyl, (C₃-C₆)-cycloalkyl and (C₁-C₄)-alkoxy is optionally substituted with up to 5 fluorine atoms,
R² represents a group selected from hydrogen or (C₁-C₄)-alkyl,
   wherein said (C₁-C₄)-alkyl is optionally substituted with up to five halogen atoms,
A represents a group selected from 5-membered aza-heteroaryl,
   wherein said 5-membered aza-heteroaryl is optionally substituted with one, two or three groups independently selected from halogen, cyano, hydroxy, methylsulfonyl, (C₃-C₆)-cycloalkyl, (C₁-C₄)-alkyl and (C₁-C₄)-alkoxy,
   wherein each said (C₁-C₄)-alkyl and (C₁-C₄)-alkoxy is optionally substituted with up to three fluorine atoms,
Z represents a group selected from Phenyl and 5- to 6-membered heteroaryl,
   optionally substituted with one, two or three groups independently selected from halogen, cyano, hydroxy, (C₁-C₄)-alkylsulfonyl, (C₃-C₆)-cycloalkyl, (C₁-C₄)-alkyl and (C₁-C₄)-alkoxy,
      wherein each said (C₁-C₄)-alkyl and (C₁-C₄)-alkoxy is optionally substituted with (C₃-C₆)-cycloalkyl and optionally substituted with up to five fluorine atoms,
         and pharmaceutically acceptable salts thereof, solvates thereof and the solvates of the salts thereof.

Preference is further given to compounds of formula (I) in which
R¹ represents a group selected from hydrogen, (C₁-C₆)-alkyl, (C₃-C₆)-cycloalkyl, 5- to 6-membered heterocycloalkyl, 5- to 6-membered heteroaryl and phenyl
   wherein said (C₁-C₆-alkyl (C₃-C₆)-cycloalkyl, 5- to 6-membered heterocycloalkyl, 5- to 6-membered heteroaryl and phenyl are optionally substituted with one or two groups independently selected from cyano, halogen, amino, hydroxy, oxo, (C₁-C₃)-alkyl, (C₁-C₄)-alkoxy, (C₁-C₄)-alkylcarbonyl, mono-(C₁-C₄)-alkylamino, di-(C₁-C₄)-alkylamino, (C₁-C₄)-alkylsulfonyl, (C₃-C₆)-cycloalkyl,
   wherein each said (C₁-C₃)-alkyl, (C₃-C₆)-cycloalkyl and (C₁-C₄)-alkoxy is optionally substituted with up to 5 fluorine atoms,
R² represents a group selected from hydrogen or methyl
A represents a group selected from triazolyl, oxazolyl, isoxazolyl, pyrazolyl, thiazolyl, 1,2,4-oxadiazolyl and 1,3,4-oxadiazolyl
   optionally substituted with one, two or three groups independently selected from halogen, cyano, hydroxy, methylsulfonyl, (C₃-C₆)-cycloalkyl, (C₁-C₄)-alkyl, and (C₁-C₄)-alkoxy,
   wherein each said (C₁-C₄)-alkyl, (C₁-C₄)-alkoxy is optionally substituted with up to three fluorine atoms,
Z represents a group selected from Phenyl and 5 to 6 membered heteroaryl,
   optionally substituted, with one, two or three groups independently selected from halogen, cyano, hydroxy, methylsulfonyl, (C₃-C₆)-cycloalkyl, (C₁-C₄)-alkyl, (C₁-C₄)-alkoxy,
      wherein each said (C₁-C₄)-alkyl and (C₁-C₄)-alkoxy is optionally substituted with, (C₃-C₆)-cycloalkyl and optionally substituted with up to five fluorine atoms,
and pharmaceutically acceptable salts thereof, solvates thereof and the solvates of the salts thereof.

Preference is further given to compounds of the formula (I) in which
R¹ represents a group selected from hydrogen, (C₁-C₆-alkyl, (C₃-C₆)-cycloalkyl, 5- to 6-membered heterocycloalkyl, 5- to 6-membered heteroaryl and phenyl
   wherein said (C₁-C₆)-alkyl (C₃-C₆)-cycloalkyl, 5- to 6-membered heterocycloalkyl, 5- to 6-membered heteroaryl and phenyl are optionally substituted, with one or two groups independently selected from cyano, halogen, amino, hydroxy, oxo, C₁-C₃-alkyl, (C₁-C₄)-alkoxy, (C₁-C₄)-alkylcarbonyl, mono-(C₁-C₄)-alkylamino, di-(C₁-C₄)-alkylamino, (C₁-C₄)-alkylsulfonyl, (C₃-C₆)-cycloalkyl,
   wherein each said C₁-C₃-alkyl, (C₃-C₆)-cycloalkyl, (C₁-C₄)-alkoxy is optionally substituted with up to 5 fluorine atoms
R² represents a group selected from hydrogen or methyl
A represents a group selected from triazolyl, oxazolyl, isoxazolyl, pyrazolyl, thiazolyl, 1,2,4-oxadiazolyl and 1,3,4-oxadiazolyl
   optionally substituted with one, two or three groups independently selected from halogen, cyano, hydroxy, methylsulfonyl, (C₃-C₆)-cycloalkyl, (C₁-C₄)-alkyl and (C₁-C₄)-alkoxy
   wherein each said (C₁-C₄)-alkyl, (C₁-C₄)-alkoxy is optionally substituted with up to three fluorine atoms,
   with the provisio that for A representing thiazolyl or pyrazolyl R¹ will not represent methylimidazol
Z represents a group selected from Phenyl and 5- to 6-membered heteroaryl,
   optionally substituted with one, two or three groups independently selected from halogen, cyano, hydroxy, methylsulfonyl, (C₃-C₆)-cycloalkyl, (C₁-C₄)-alkyl, and (C₁-C₄)-alkoxy,
      wherein each said (C₁-C₄)-alkyl and (C₁-C₄)-alkoxy is optionally substituted with (C₃-C₆)-cycloalkyl and optionally substituted with up to five fluorine atoms,
and pharmaceutically acceptable salts thereof, solvates thereof and the solvates of the salts thereof.

Preference is further given to compounds of the formula (I) in which
R¹ represents a group selected from hydrogen, (C₁-C₆-alkyl, (C₃-C₆)-cycloalkyl, 5- to 6-membered heterocycloalkyl, 5- to 6-membered heteroaryl and phenyl
   wherein said (C₁-C₆-alkyl (C₃-C₆)-cycloalkyl, 5- to 6-membered heterocycloalkyl, 5- to 6-membered heteroaryl and phenyl are optionally substituted, with one or two groups independently selected from cyano, halogen, amino, hydroxy, oxo, C₁-C₃-alkyl, (C₁-C₄)-alkoxy, (C₁-C₄)-alkylcarbonylmono-(C₁-C₄)-alkylamino, di-(C₁-C₄)-alkylamino, (C₁-C₄)-alkylsulfonyl, (C₃-C₆)-cycloalkyl,
   wherein each said (C₁-C₃)-alkyl, (C₃-C₆)-cycloalkyl, and (C₁-C₄)-alkoxy is optionally substituted with up to 5 fluorine atoms,
R² represents a group selected from hydrogen or methyl,
A represents a triazolyl
   optionally substituted with one, two or three groups independently selected from halogen, cyano, hydroxy, methylsulfonyl, (C₃-C₆)-cycloalkyl, (C₁-C₄)-alkyl, and (C₁-C₄)-alkoxy,
   wherein each said (C₁-C₄)-alkyl and (C₁-C₄)-alkoxy is optionally substituted with up to three fluorine atoms,
Z represents a group selected from Phenyl and 5 to 6 membered heteroaryl,
   optionally substituted, with one, two or three groups independently selected from halogen, cyano, hydroxy, methylsulfonyl, (C₃-C₆)-cycloalkyl, (C₁-C₄)-alkyl and (C₁-C₄)-alkoxy,
      wherein each said (C₁-C₄)-alkyl and (C₁-C₄)-alkoxy is optionally substituted with (C₃-C₆)-cycloalkyl and optionally substituted with up to five fluorine atoms,
and pharmaceutically acceptable salts thereof, solvates thereof and the solvates of the salts thereof.

Preference is given to compounds of the formula (I) selected from the group consisting of
N-{[(4R)-4-cyclopropyl-2,5-dioxoimidazolidin-4-yl]methyl}-2-(2,4-difluorophenyl)-2H-1,2,3-triazole-4-carboxamide

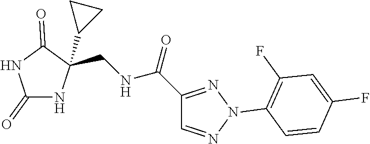 [SMILES: O=C1NC(=O)[C@](CNC(=O)c2cnn(-c3ccc(F)cc3F)n2)(C2CC2)N1]

N-{[(4R)-4-cyclopropyl-2,5-dioxoimidazolidin-4-yl]methyl}-2-(4-fluorophenyl)-2H-1,2,3-triazole-4-carboxamide

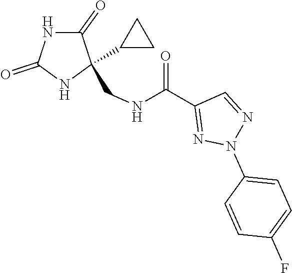 [SMILES: O=C1NC(=O)[C@](CNC(=O)c2cnn(-c3ccc(F)cc3)n2)(C2CC2)N1]

2-(3-chloro-4-fluorophenyl)-N-{[(4R)-4-cyclopropyl-2,5-dioxoimidazolidin-4-yl]methyl}-2H-1,2,3-triazole-4-carboxamide

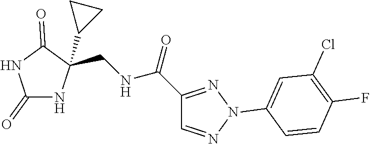 [SMILES: O=C1NC(=O)[C@](CNC(=O)c2cnn(-c3ccc(F)c(Cl)c3)n2)(C2CC2)N1]

ent-N-[(2,5-dioxo-4-(propan-2-yl)imidazolidin-4-yl)methyl]-2-(4-fluorophenyl)-2H-1,2,3-triazole-4-carboxamide

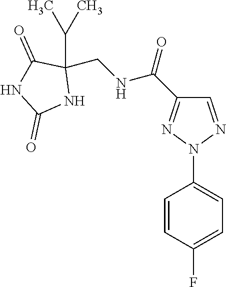 [SMILES: CC(C)C1(CNC(=O)c2cnn(-c3ccc(F)cc3)n2)NC(=O)NC1=O]

ent-N-[(4-cyclobutyl-2,5-dioxoimidazolidin-4-yl)methyl]-2-(4-fluorophenyl)-2H-1,2,3-triazole-4-carboxamide

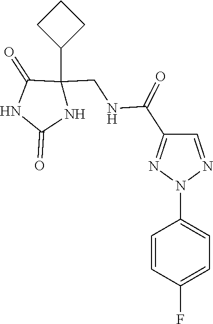 [SMILES: O=C1NC(=O)C(CNC(=O)c2cnn(-c3ccc(F)cc3)n2)(C2CCC2)N1]

ent-N-[(4-cyclobutyl-2,5-dioxoimidazolidin-4-yl)methyl]-2-(3,4,5-trifluorophenyl)-2H-1,2,3-triazole-4-carboxamide

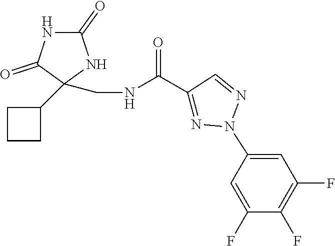 [SMILES: O=C1NC(=O)C(CNC(=O)c2cnn(-c3cc(F)c(F)c(F)c3)n2)(C2CCC2)N1]

ent-N-[(4-cyclobutyl-2,5-dioxoimidazolidin-4-yl)methyl]-2-(3-fluorophenyl)-2H-1,2,3-triazole-4-carboxamide

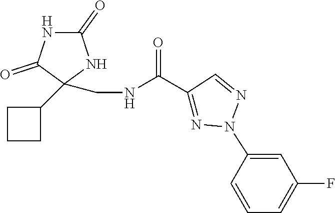 [SMILES: O=C1NC(=O)C(CNC(=O)c2cnn(-c3cccc(F)c3)n2)(C2CCC2)N1]

and
ent-N-[(4-cyclobutyl-2,5-dioxoimidazolidin-4-yl)methyl]-2-(3,4-difluorophenyl)-2H-1,2,3-triazole-4-carboxamide

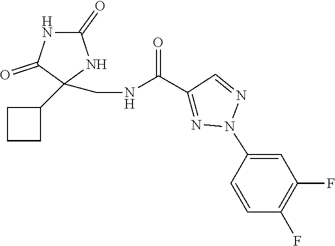 [SMILES: O=C1NC(=O)C(CNC(=O)c2cnn(-c3ccc(F)c(F)c3)n2)(C2CCC2)N1]

and pharmaceutically acceptable salts thereof, solvates thereof and the solvates of the salts thereof.
Preference is also given to compounds of formula (I) in which R¹ represents cyclopropyl, cyclobutyl, isopropyl or methylimidazole.
Particular preference is also given to compounds of formula (I) in which R¹ represents cyclopropyl.

Preference is also given to compounds of formula (I) in which R² represents hydrogen or methyl. Particular preference is also given to compounds of formula (I) in which R² represents hydrogen. Preference is also given to compounds of formula (I) in which A represents a triazole, an isoxazole, an oxazole, a pyrazole or an oxadiazole.

Particular preference is also given to compounds of formula (I) in which A represents a triazole.
Preference is also given to compounds of formula (I) in which Z represents phenyl optionally substituted with one, two or three groups independently selected from halogen, cyano, hydroxy, (C₁-C₄)-alkoxy, trifluoromethoxy and difluoromethoxy.
Particular preference is given to compounds of formula (I) in which Z represents phenyl substituted with 1 to 3 halogens, preferably independently selected from chlorine or fluorine.

Particular preference is also given to compounds of formula (I) in which Z represents p-fluorophenyl.

Another object of the present invention is a process for the preparation of the compounds of formula (I) characterized by reacting a compound of formula (II)

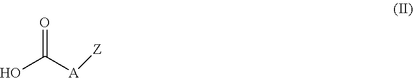

in which A and Z have the meaning given above, in an inert solvent with a condensing agent such as 1-(3-dimethylaminopropyl)-3-ethylcarbodiimide hydrochloride in the presence of a base such as N,N-diisopropylethylamine and an auxiliary reagent such as HOBt with a compound of formula (III) which may be used as salt (e.g., hydrochloride)

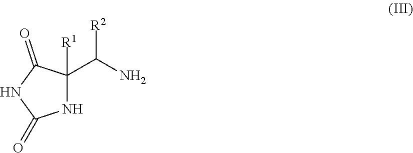

in which R¹ and R² have the meaning given above.

Inert solvents for process step (II)+(III)→(I) include, for example, halogenated hydrocarbons, such as dichloromethane, trichlorethylene, chloroform or chlorobenzene, ethers, such as diethyl ether, dioxane, tetrahydrofuran, 1,2-dimethoxyethane or diglyme, hydrocarbons such as benzene, toluene, xylene, hexane, cyclohexane or petroleum fractions or other solvents such as acetonitrile, N,N-dimethylformamide, N,N-dimethylacetamide, dimethyl sulfoxide, N,N-dimethylpropyleneurea (DMPU), N-methyl-2-pyrrolidone (NMP) or pyridine. It is also possible to use mixtures of the said solvents. Preference is given to using dichloromethane or N,N-dimethylformamide. Dichloromethane is particularly preferably used.

Suitable condensing agents for amide formation in process step (II)+(III)→(I) include, for example, carbodiimides such as N,N′-diethyl, N,N′-dipropyl, N,N′-diisopropyl, N,N′-dicyclohexylcarbodiimide (DCC) or N-(3-dimethylamino-propyl)-N′-ethylcarbodiimide hydrochloride (EDC), phosgene derivatives such as N, N′-carbonyldiimidazole (CDI), 1,2-oxazolium compounds such as 2-ethyl-5-phenyl-1,2-oxazolium-3-sulfate or 2-tert-butyl-5-methyl-isoxazolinium perchlorate, acylamino compounds such as 2-ethoxy-1-ethoxy-carbonyl-1,2-dihydrohydroquinoline, or isobutyl chloroformate, propanephosphonic acid anhydride (T3P), 1-chloro-N,N,2-trimethylprop-1-en-1-amine, cyanophosphonic acid diethyl ester, (2-oxo-3-oxazolidinyl)-phosphoryl chloride, benzotriazazol-1-yloxy-tris (dimethylamino) phosphonium-hexafluorophosphate, benzotriazol-1-yl-oxytripyrrolidinophosphonium hexafluorophosphate (PyBOP), 2-(1H-Benzotriazole-1-yl)-1,1,3,3-tetramethylaminium tetrafluoroborate (TBTU), 2-(1H-benzotriazol-1-yl)-1,1,3,3-tetramethyluronium hexafluorophosphate (HBTU), O-(1,2-Dihydro-2-oxo-1-pyridyl)-N,N,N′—N′-tetramethyluronium tetrafluoroborate (TPTU), 1-[Bis(dimethylamino)methylene]-1H-1,2,3-triazolo[4,5-b]pyridinium 3-oxid hexafluorophosphate (HATU) or O-(6-Chloro-1-hydrocibenzotriazol-1-yl)-1,1,3,3-tetramethyluronium tetrafluoroborate (TCTU), if appropriate in combination with other excipients such as 1 hydroxybenzotriazole (HOBt), 1-hydroxy-7-azabenzotriazole (HOAt) or N-hydroxysuccinimide (HOSu). Preferably EDC and HATU is used. Particular preference is given to using EDC.

Suitable bases for amide formation in process step (II)+(III)→(I) include, for example, alkali metal carbonates, e.g., sodium or potassium carbonate or hydrogen carbonate, or organic bases such as trialkylamines, e.g., triethylamine (TEA), trimethylamine, N methylmorpholine, N-methylpiperidine or N,N-diisopropylethylamine (DIPEA) or 4-(dimethylamino) pyridine (DMAP). Preferably, DIPEA, trimethylamine or TEA is used. Particular preference is given to using DIPEA.

The condensation (II)+(III)→(I) is generally carried out in a temperature range from −20° C. to +100° C., preferably at 0° C. to +60° C. The reaction can be carried out at normal, elevated or reduced pressure (for example from 0.5 to 5 bar). In general, one works at room temperature and normal pressure.

Alternatively, the carboxylic acid of the formula (II) can also first be converted into the corresponding carboxylic acid chloride and then reacted directly or in a separate reaction with a compound of the formula (III) to give the compounds according to the invention. The formation of carboxylic acid chlorides from carboxylic acids is carried out by the methods known to those skilled in the art, for example, by treating (II) or the corresponding carboxylate with thionyl chloride, sulfuryl chloride or oxalyl chloride in the presence of a suitable base, for example in the presence of pyridine, and optionally with the addition of dimethylformamide, optionally in a suitable inert solvent.

The compounds used are commercially available, known from the literature or can be prepared analogously to processes known from the literature.

The general procedure is exemplified by the scheme below (Scheme 1)

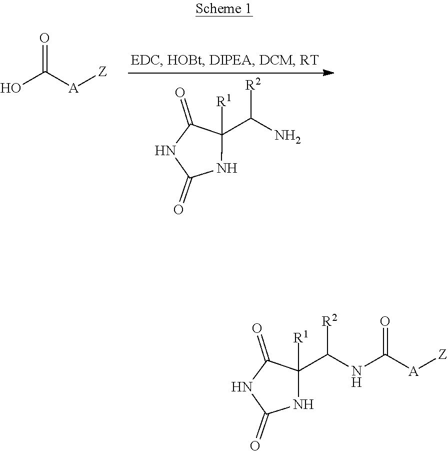

wherein A, Z, R¹ and R² are as defined above.

Amines of the formula (III) can be prepared by the process exemplified in Scheme 2

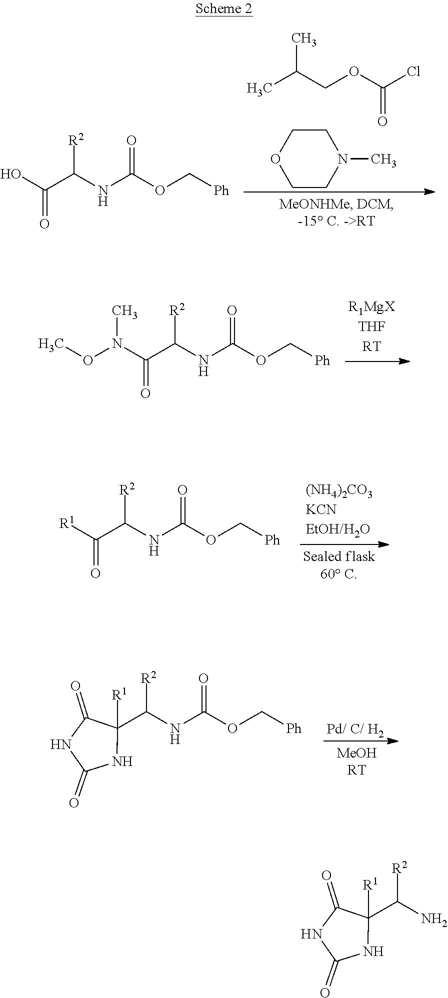

wherein R¹ is

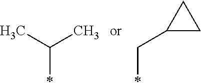

R² is as defined above

### X is Cl or Br

and * represents the point of attachment.

An alternative process variant is shown in Scheme 3

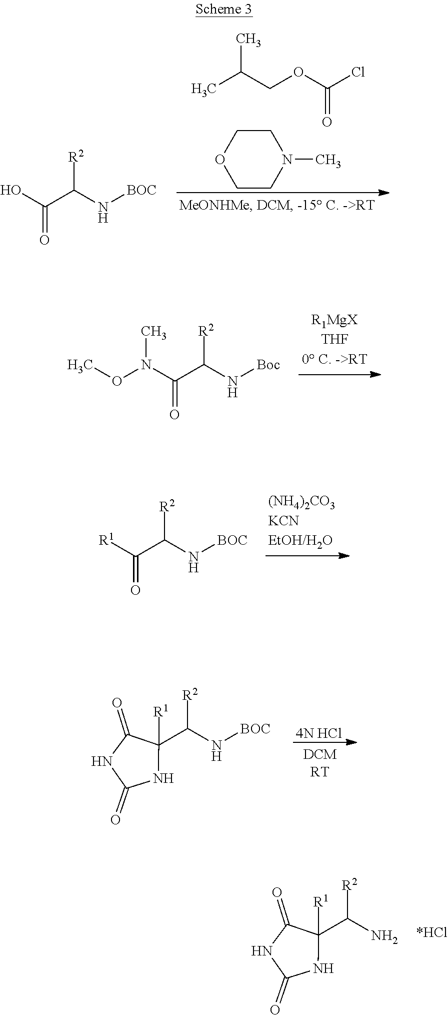

wherein R¹ is

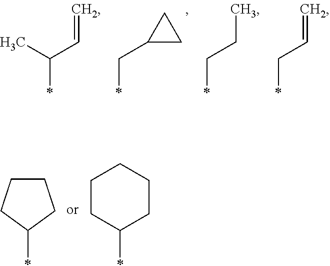

R² is as defined above

### X is Cl or Br

and * represents the point of attachment.

Another alternative process variant is shown in Scheme 4

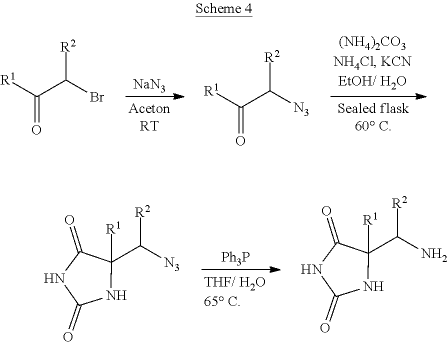

wherein R¹ is

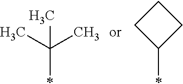

R² is as defined above
and * represents the point of attachment.

Another alternative process variant is shown in Scheme 5

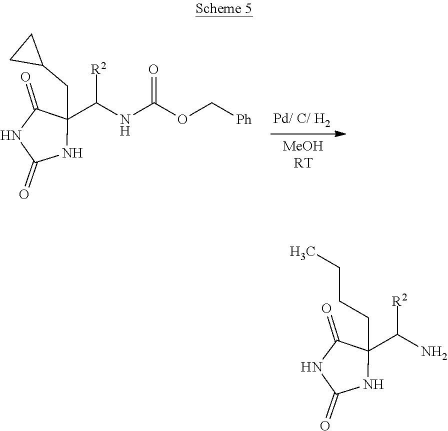

wherein R² is as defined above.

Another alternative process variant is shown in Scheme 8

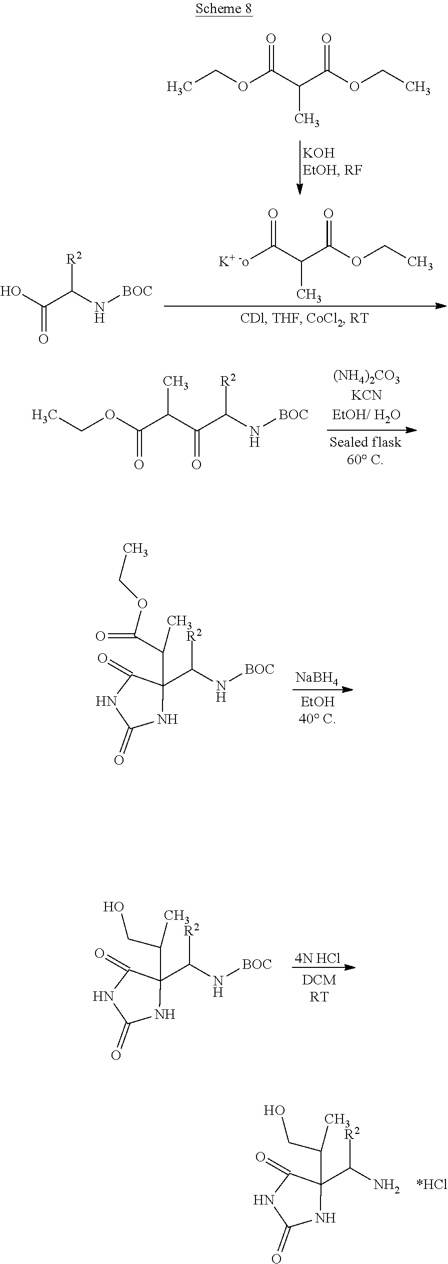

wherein R² is as defined above.

Another alternative process variant is shown in Scheme 9

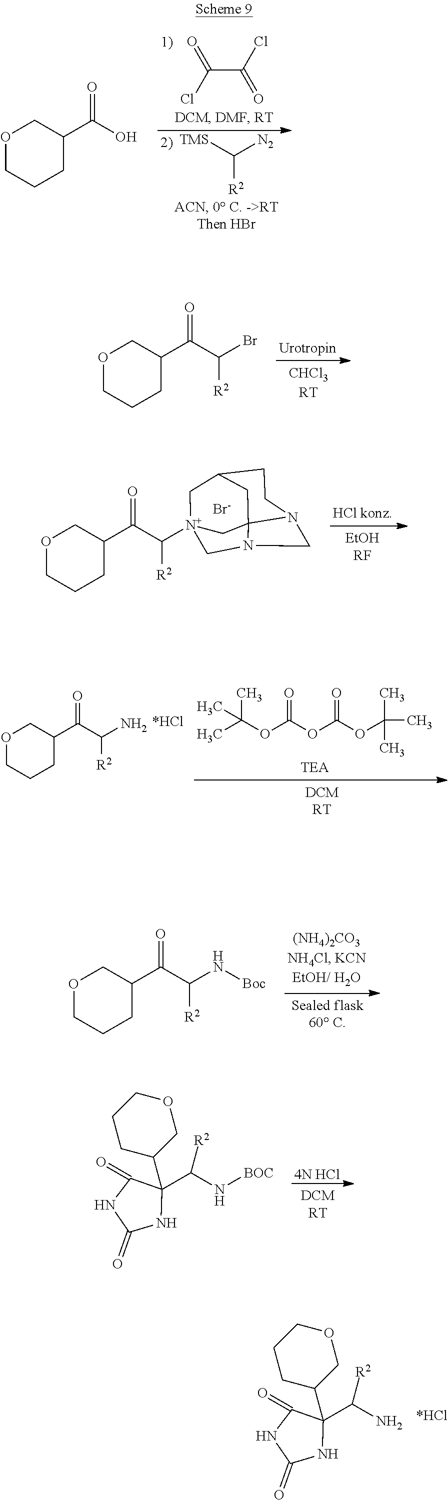

wherein R² is as defined above.

Another alternative process variant is shown in Scheme 10

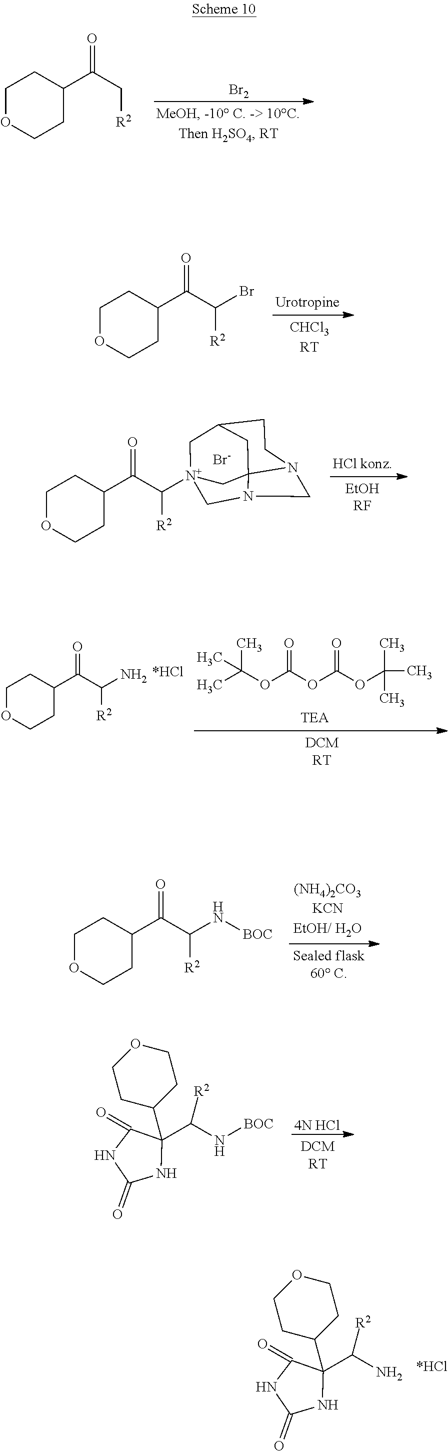

wherein R² is as defined above.

Another alternative process variant is shown in Scheme 11

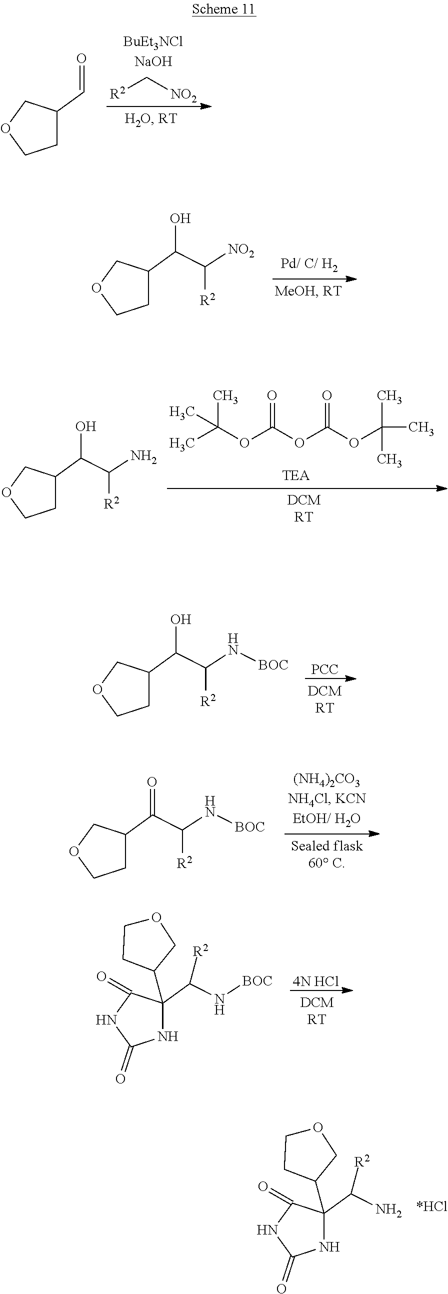

wherein R² is as defined above.

Another alternative process variant is shown in Scheme 12

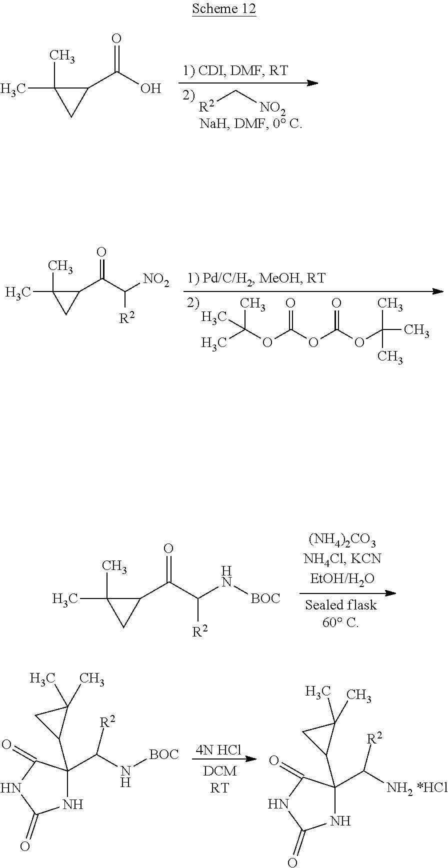

wherein R² is as defined above.

Another alternative process variant is shown in Scheme 13

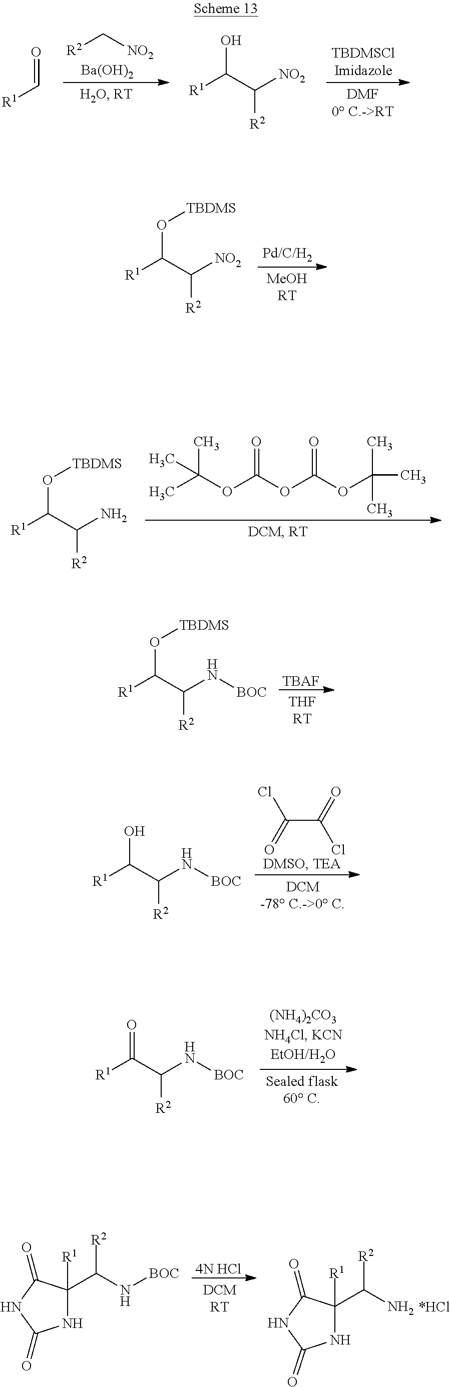

wherein R¹ is

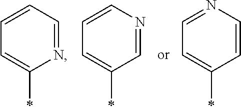

R² is as defined above
and * represents the point of attachment.

Another alternative process variant is shown in Scheme 14

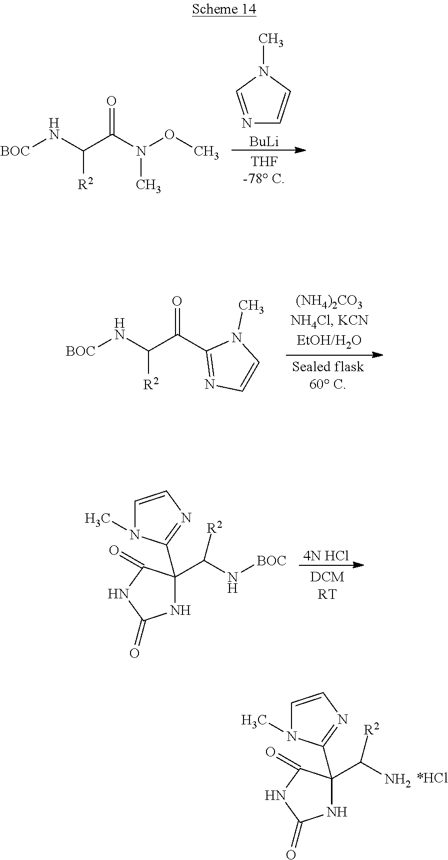

wherein R² is as defined above.

Another alternative process variant is shown in Scheme 15

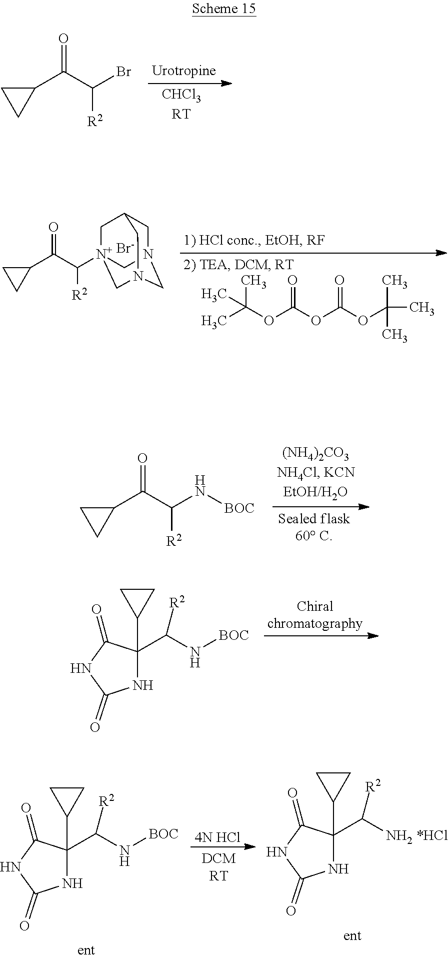

wherein R² is as defined above.

Another alternative process variant is shown in Scheme 16.

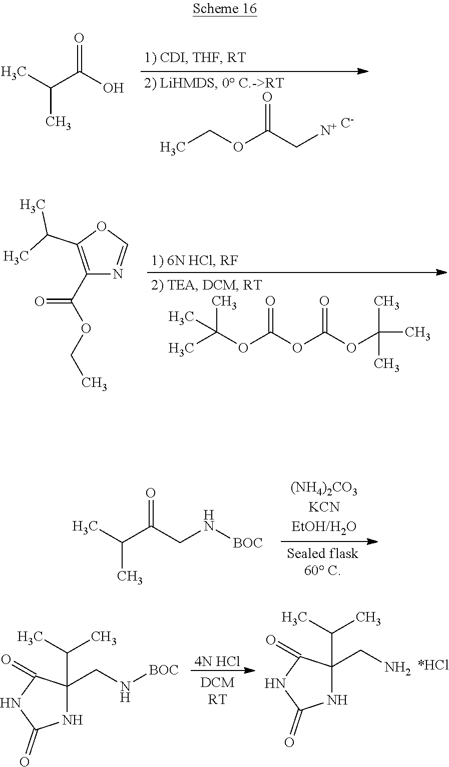

Another alternative process variant is shown in Scheme 17

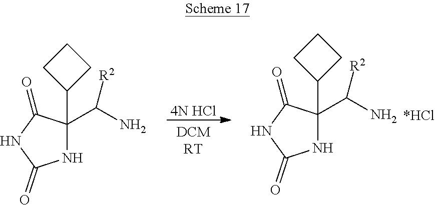

wherein R² is as defined above.

Another alternative process variant is shown in Scheme 18

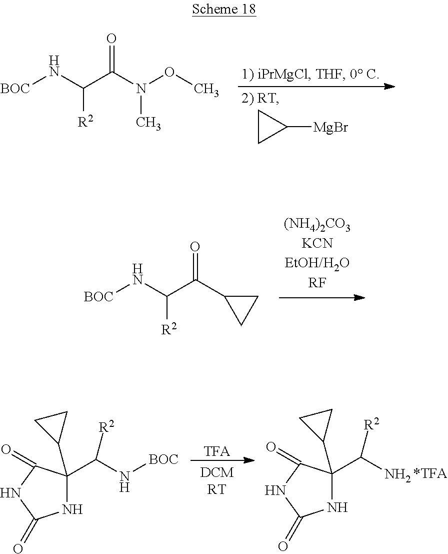

wherein R² is as defined above.

Acids of formula (II) can be prepared by the method exemplified in Scheme 19

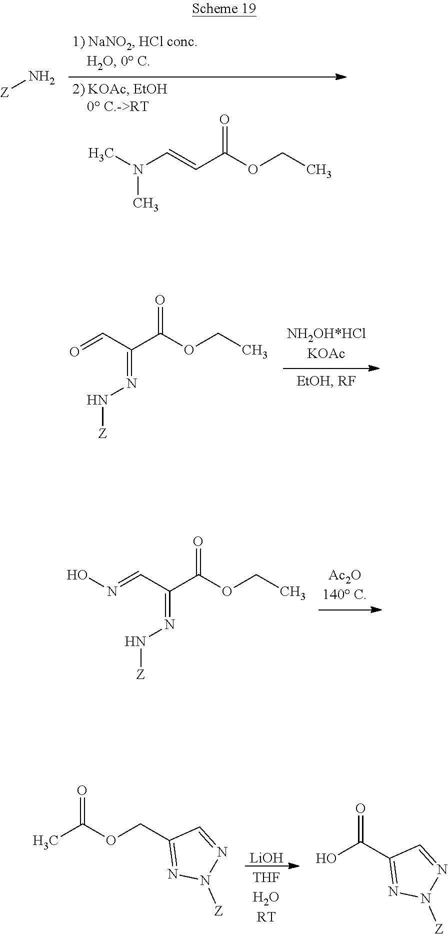

wherein Z is

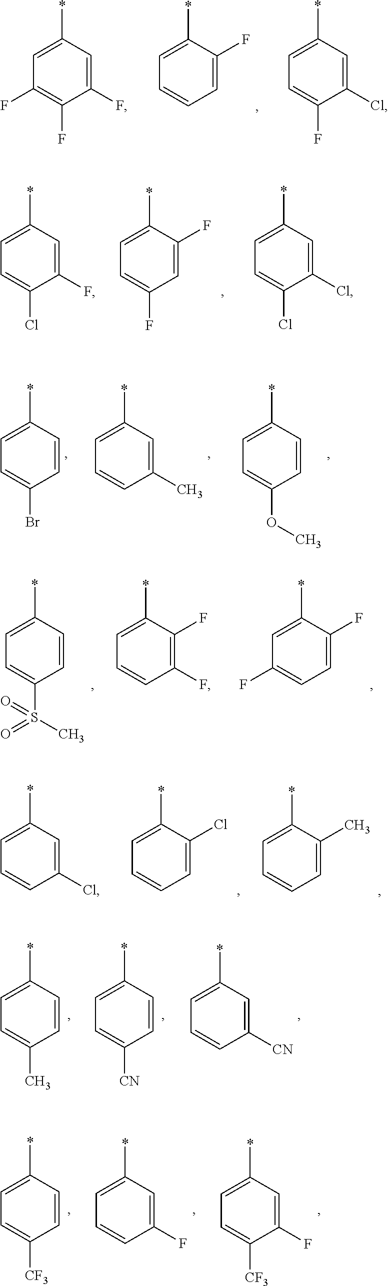

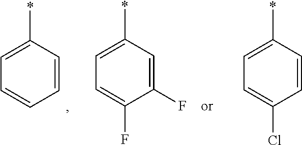

and * represents the point of attachment.

An alternative process variant is shown in Scheme 20.

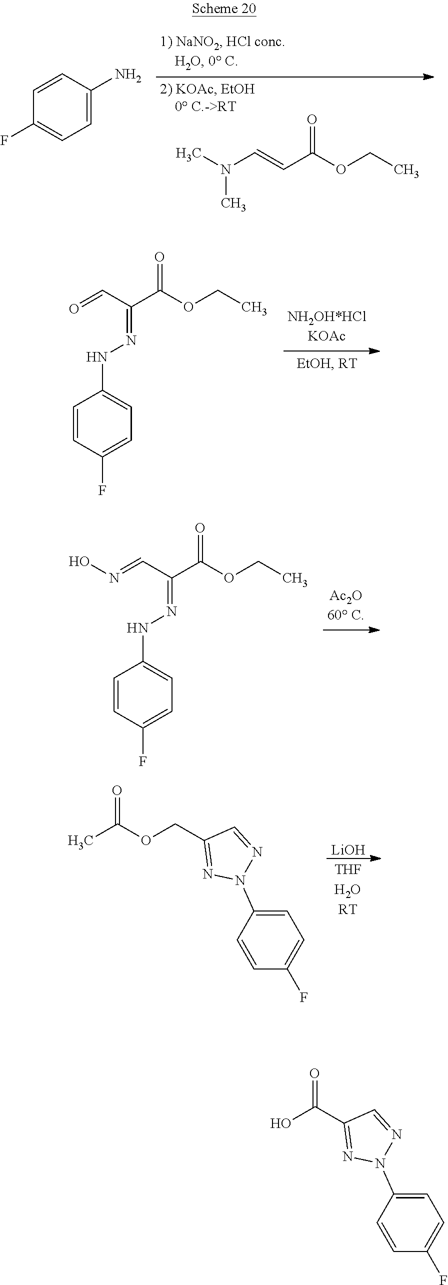

Another alternative process variant is shown in Scheme 21

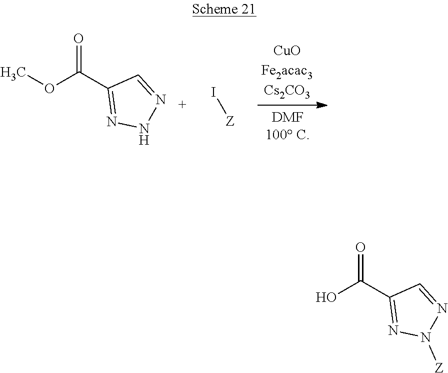

wherein Z is

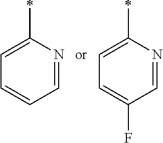

and * represents the point of attachment.

Another alternative process variant is shown in Scheme 22.

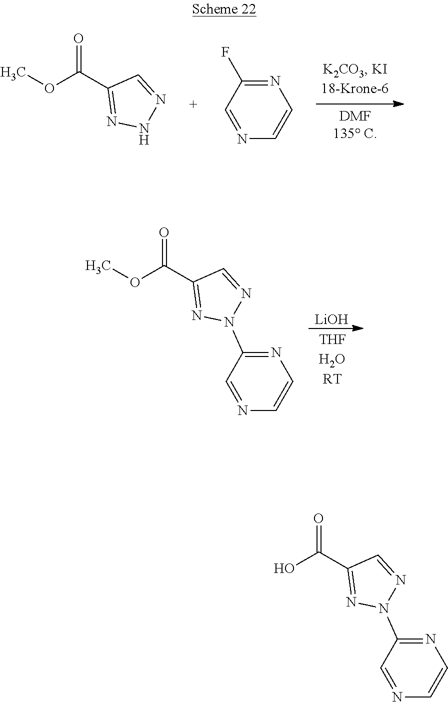

Another alternative process variant is shown in Scheme 23.

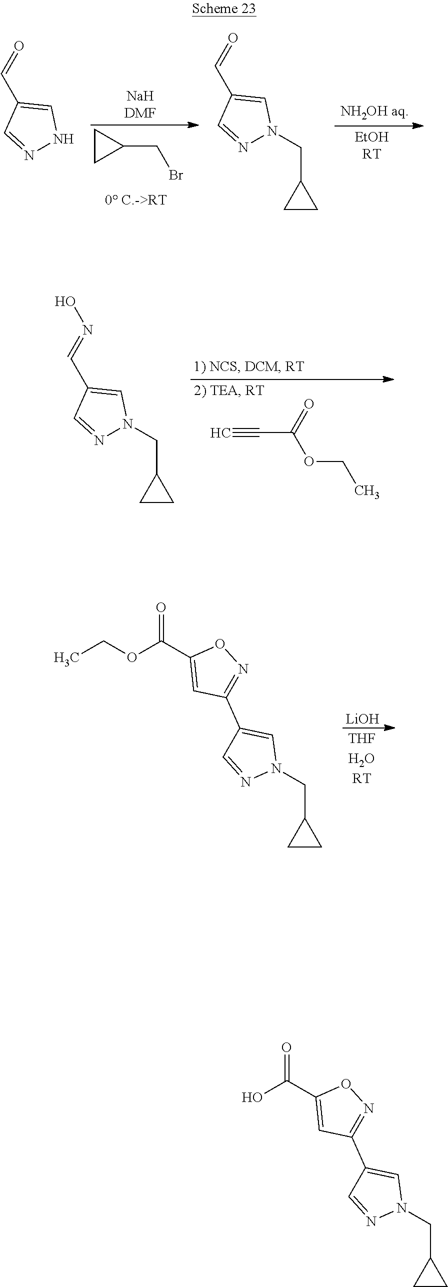

Another alternative process variant is shown in Scheme 24.

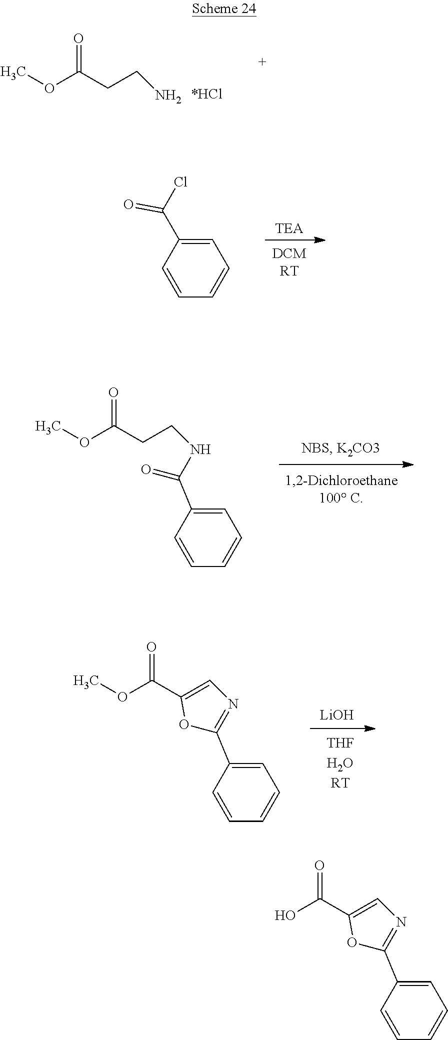

Another alternative process variant is shown in Scheme 25.

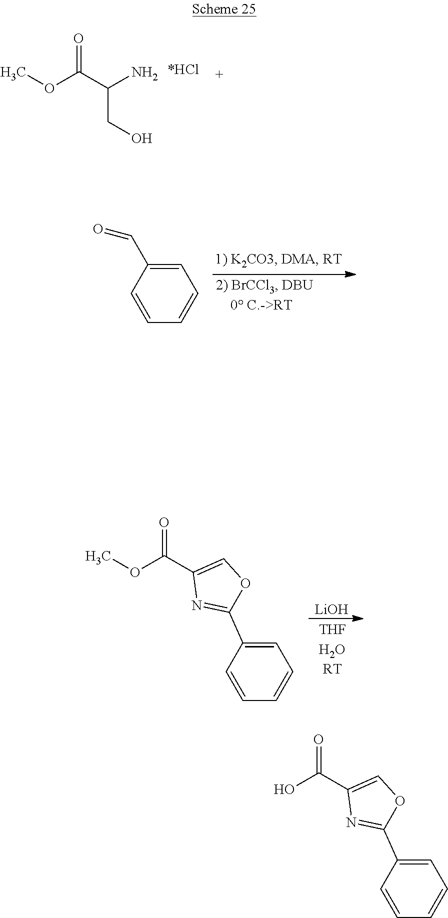

Another alternative process variant is shown in Scheme 26.

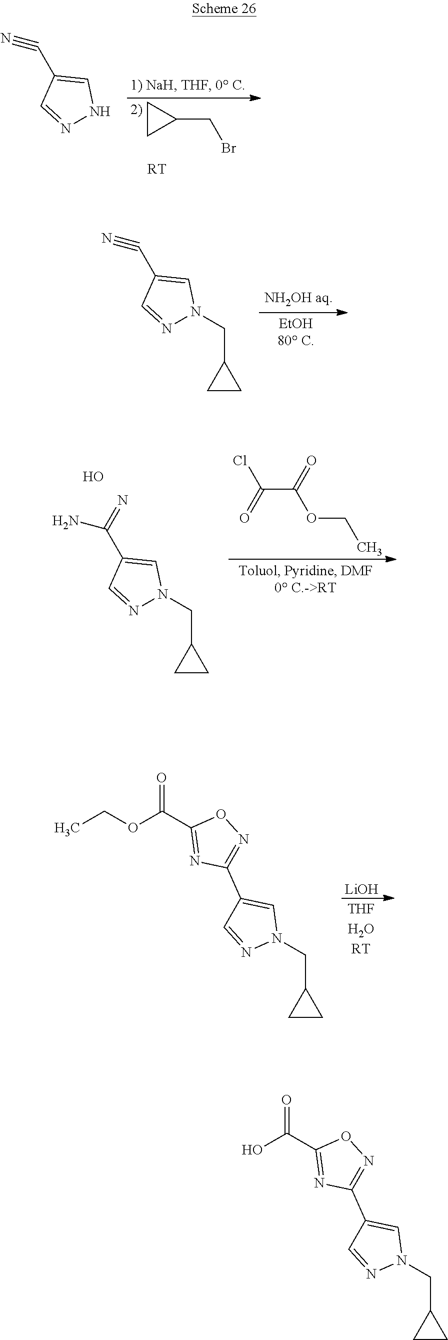

Another alternative process variant is shown in Scheme 27

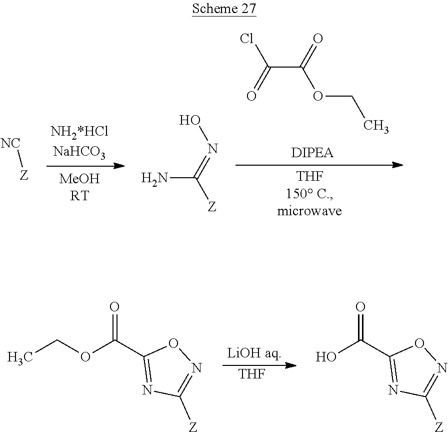

wherein Z is

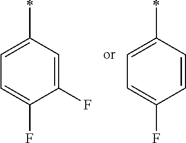

and * represents the point of attachment.

Detailed instructions are also to be found in the experimental part in the section for preparing the starting compounds and intermediates.

Compounds of the current invention possess valuable pharmacological properties and can be used for the treatment and/or prophylaxis of diseases/disease state affecting human beings aid animals.

Compounds concerning the present invention are potent, chemically stable antagonists of the ADAMTS7 metalloprotease and are suited for the treatment and/or prevention of diseases in human beings and animals such as, heart diseases, vascular diseases, and/or cardiovascular diseases, including atherosclerosis, coronary artery disease (CAD), peripheral vascular disease (PAD)/arterial occlusive disease and/or restenosis after angioplasty (including the use of drug-coated or non drug-coated balloons and/or stent-implantation) and/or for the treatment and/or prophylaxis of lung diseases, inflammatory diseases, fibrotic diseases, metabolic diseases, cardiometabolic diseases and/or diseases/disease states affecting the kidneys and/or the central nervous and/or neurological system as well as gastrointestinal and/or urologic and/or ophthalmologic diseases/disease states.

In the context of the present invention heart diseases, vascular diseases and/or cardiovascular diseases or disease of the cardiovascular system include, e.g., acute and chronic heart failure, arterial hypertension, coronary heart disease, stable and instable angina pectoris, myocardial ischemia, myocardial infarction, coronary microvascular dysfunction, microvascular obstruction, no-reflow-phenomenon, shock, atherosclerosis, coronary artery disease, peripheral artery disease, peripheral arterial disease, intermittent claudication, severe intermittent claudication, limb ischemia, critical limb ischemia, hypertrophy of the heart, cardiomyopathies of any etiology (such as, e.g., dilatative cardiomyopathy, restrictive cardiomyopathy, hypertrophic cardiomyopathy, ischemic cardiomyopathy), fibrosis of the heart, atrial and ventricular arrhythmias, transitory and/or ischemic attacks, apoplexy, ischemic and/or hemorrhagic stroke, preeclampsia, inflammatory cardiovascular diseases, metabolic diseases, diabetes, type-I-diabetes, type-II-diabetes, diabetes mellitus, peripheral and autonomic neuropathies, diabetic neuropathies, diabetic microangiopathies, diabetic retinopathy, diabetic ulcera at the extremities, gangrene, CREST-syndrome, hypercholesterolemia, hypertriglyceridemia, lipometabolic disorder, metabolic syndrome, increased levels of fibrinogen and low-density lipoproteins (i.e. LDL), increased concentrations of plasminogen-activator inhibitor1 (PAI-1), as well as peripheral vascular and cardiac vascular diseases, peripheral circulatory disorders, primary and secondary Raynaud syndrome, disturbances of the microcirculation, arterial pulmonary hypertension, spasms of coronary and peripheral arteries, thromboses, thromboembolic diseases, edema-formation, such as pulmonary edema, brain-edema, renal edema, myocardial edema, myocardial edema associated with heart failure, restenosis after i.e. thrombolytic therapies, percutaneous-transluminal angioplasties (PTA), transluminal coronary angioplasties (PTCA), heart transplantations, bypass-surgeries as well as micro- and macrovascular injuries (e.g., vasculitis), reperfusion-damage, arterial and venous thromboses, microalbuminuria, cardiac insufficiency, endothelial dysfunction.

In the light of the present invention, heart failure includes more specific or related kinds of diseases such as acute decompensated heart failure, right heart failure, left heart failure, global insufficiency, ischemic cardiomyopathy, dilatative cardiomyopathy, congenital heart defect(s), valve diseases, heart failure related to valve diseases, mitral valve stenosis, mitral valve insufficiency, aortic valve stenosis, aortic valve insufficiency, tricuspid valve stenosis, tricuspid valve insufficiency, pulmonary valve stenosis, pulmonary valve insufficiency, combined valvular defects, inflammation of the heart muscle (myocarditis), chronic myocarditis, acute myocarditis, viral myocarditis, bacterial myocarditis, diabetic heart failure, alcohol-toxic cardiomyopathy, cardiac storage diseases, heart failure with preserved ejection fraction (HFpEF), diastolic heat failure, heart failure with reduced ejection fraction (HFrEF), systolic heart failure.

In the context of the present invention, the terms atrial arrhythmias and ventricular arrhythmias also include more specific and related disease-entitites, such as: Atrial fibrillation, paroxysmal atrial fibrillation, intermittent atrial fibrillation, persistent atrial fibrillation, permanent atrial fibrillation, atrial flutter, sinus arrhythmia, sinus tachycardia, passive heterotopy, active heterotopy, replacement systoles, extrasystoles, disturbances in the conduction of impulses, sick-sinus syndrome, hypersensitive carotis-sinus, tachycardias, AV-node re-entry tachycardias, atrioventricular re-entry tachycardia, WPW-syndrome (Wolff-Parkinson-White syndrome), Mahaim-tachycardia, hidden accessory pathways/tracts, permanent junctional re-entry tachycardia, focal atrial tachycardia, junctional ectopic tachycardia, atrial re-entry tachycardia, ventricular tachycardia, ventricular flutter, ventricular fibrillation, sudden cardiac death.

In the context of the present invention, the term coronary heart disease also includes more specific or related diseases entities, such as: Ischemic heart disease, stable angina pectoris, acute coronary syndrome, instable angina pectoris, NSTEMI (non-ST-segement-elevation myocardial infarction), STEMI (ST-segement-elevation myocardial infarction), ischemic damage of the heart, arrhythmias, and myocardial infarction.

In the context of the present invention, diseases of the central nervous and neurological system or central nervous and neurological diseases/diseases states refer to, e.g., the following diseases/diseases states: Transitory and ischemic attacks, stroke/apoplexy, ischemic and hemorrhagic stroke, depression, anxiety disorder, post-traumatic stress-disorder, poly-neuropathy, diabetic poly-neuropathy, stress-induced hypertension.

Compounds concerning the present invention are furthermore suited for the prophylaxis and/or treatment of poly-cystic kidney-disease (PCKD) and the syndrome of inadequate ADH-secretion (SIADH). Furthermore, the compounds described in the present invention are suited for the treatment and/or prophylaxis of kidney diseases, especially of acute and chronic renal insufficiency as well as of acute and chronic renal failure.

In the context of the present invention, the term acute renal insufficiency/renal failure includes acute presentations of kidney diseases, kidney failure and/or renal insufficiency with or without the dependency on dialysis as well as underlying or related kidney diseases such as renal hypoperfusion, hypotension during dialysis, lack of volume (i.e. dehydration, blood-loss), shock, acute glomerulonephritis, hemolytic-uremic syndrome (HUS), vascular catastrophe (arterial or venous thrombosis or embolism), cholesterol-embolism, acute Bence-Jones-kidney associated with plasmacytoma, acute supravesical or subvesical outlow obstructions, immunologic kidney diseases such as kidney transplant rejection, immuncomplex-induced kidney diseases, tubular dilatation, hyperphosphatemia and/or akute kidney diseases which may be characterized by the need for dialysis. Also included are conditions of partial nephrectomy, dehydration caused by force diuresis, uncontrolled increase in blood pressure accompanied by malignant hypertension, urinary tract obstructions and infections and amyloidosis as well as systemic disorders with glomerular participation such as rheumatologic-immunologic systemic disorders, such as Lupus erythematodes, renal artery thrombosis, renal vein thrombosis, analgesics-induced nephropathy and renal-tubular acidosis as well as radio-opaque substance—as well as drug-induced acute interstitial kidney diseases.

In the context of the present invention the term chronic renal insufficiency/chronic renal failure includes chronic manifestations/presentations of kidney diseases, renal failure and/or renal insufficiency with and without the dependency on dialysis as well as underlying or related kidney diseases such as renal hypoperfusion, hypotension during dialysis, obstructive uropathy, glomerulopathies, glomerular and tubular proteinuria, renal edema, hematuria, primary, secondary as well as chronic glomerulonephritis, membraneous and membraneous-proliferative glomerulonephritis, Alport-syndrome, glomerulosclerosis, tubulointerstitial diseases, nephropathic diseases such as primary and hereditary kidney disease(s), renal inflammation, immunologic kidney diseases such as transplant rejection, immuncomplex-induced kidney diseases, diabetic and non-diabetic nephropathy, pyelonephritis, renal cysts, nephrosclerosis, hypertensive nephrosderosis and nephrotic syndrome, which are diagnostically characterized by i.e. abnormally reduced creatinine- and/or water-excretion, abnormally increased blood-concentrations of urea, nitrogen, potassium and/or creatinine, altered activity of kidney enzymes, such as, e.g., glutamylsynthase, altered urinary osmolarity or volume, increased microalbuminuria, macroalbuminuria, lesions associated with glomeruli and arterioles, tubular dilatation, hyperphosphatemia and/or the need for dialysis; likewise included are renal cell carcinomas, conditions after partial kidney-resection, dehydration attributed to force diuresis, uncontrolled increase in blood pressure with malignant hypertension, urinary tract obstruction and urinary tract infection and amyloidosis as well as systemic diseases with glomerula participation such as rheumatologic-immunologic systemic diseases, such as lupus erythematodes, as well as renal artery stenosis, renal artery thrombosis, renal vein thrombosis, analgesics-induced nephropathy and renal-tubular acidosis. Furthermore, included are radio-opaque substance- or drug-induced chronic interstitial kidney diseases, metabolic syndrome and dyslipidemia. The current invention also includes the use of the drugs of the current invention for the treatment and/or prophylaxis of after-effects of renal insufficiency such as lung edema, heart failure, uremia, anemia, disturbances in electrolytes (e.g., hyperkalemia, hyponatremia) and disturbances in bone- and carbohydrate-metabolism.

Additionally, compounds of the current invention are suited for the treatment and/or prophylaxis of lung diseases (partially also seen as vascular diseases), such as, e.g., pulmonary arterial hypertension (PAH) and other forms of pulmonary hypertension (PH), chronic-obstructive pulmonary disease (COPD), acute respiratory distress syndrome (ARDS), acute lung injury (ALI), lung fibrosis, lung emphysema (e.g., lung emphysema induced by cigarette smoke), cystic fibrosis (CF) as well as for the treatment and/or prophylaxis of alpha-1-antitrypsin deficiency (AATD), acute coronary syndrome (ACS), inflammation of the heart muscle (myocarditis) and other autoimmune diseases of the heart (pericarditis, endocarditis, valvolitis, aortitis, cardiomyopathies), cardiogenic shock, aneurysms, sepsis (SIRS), multiple organ failure (MODS, MOF), inflammatory kidney diseases, chronic bowel diseases (IBD, Crohn's Disease, UC), pancreatitis, peritonitis, rheumatoid diseases, inflammatory skin diseases as well as inflammatory eye diseases.

Furthermore, compounds of the current invention can be used for the treatment and/or prophylaxis of asthmatic diseases of different severity with intermittent or persistent courses (refractive asthma, bronchial asthma, allergic asthma, intrinsic asthma, extrinsic asthma, asthma induced by drugs or dust), of different kinds of bronchitis (chronic bronchitis, infectious bronchitis, eosinophilic bronchitis), of bronchiolitis obliterans, bronchiectasia, pneumonia, idiopathic interstitial pneumonia, farmer's lung and related diseases, coughing and common cold diseases (chronic inflammatory cough, iatrogenic cough), inflammations of the nasal mucosa (including drug-induced rhinitis, vasomotor rhinitis and season-dependent allergic rhinitis, e.g., allergic coryza) as well as of polyps.

The compounds described in the current invention also represent active compounds for the treatment of diseases of the central nervous system, characterized by disturbances of the NO/cGMP-system. They are especially suited for improvement of perception, concentration-performance, learning-behaviour or memory-performance after cognitive disturbances as they occur with conditions/illnesses/syndromes such as “mild cognitive impairment”, age-associated learning- and memory-disturbances, age-associated memory-loss, vascular dementia, craniocerebral injury, stroke, dementia occurring after stroke (“post stroke dementia”), post-traumatic craniocerebral injury, general concentration-disturbances, concentration-disturbances affecting children with learning- and memory-problems, Alzheimer's disease, dementia with Lewy-bodies, dementia with degeneration of the frontal lobe including Pick's syndrome, Parkinson's Disease, dementia with corticobasal degeneration, amyotrophic lateral sclerosis (ALS), Huntington's Disease, demyelination, multiple sclerosis, thalamic degeneration, Creutzfeld-Jacob-dementia, HIV-dementia, schizophrenia with dementia or Korsakoff-psychosis. They are also suited for the treatment and or prevention of diseases/disease states of the central nervous system such as conditions of anxiety, tension/pressure and depressions, bipolar disorder, sexual dysfunction due to disturbances in the central nervous system as well as sleep abnormalities and for regulation of pathological disturbances of food-, luxury food- and ‘dependence causing substance’-intake.

Furthermore, the compounds of the current invention are also suited for the treatment and/or prophylaxis of urologic diseases/disease states such as, e.g., urinary incontinence, stress-induced incontinence, urge incontinence, reflex incontinence and overflow incontinence, detrusor hyperactivity, neurogenic detrusor hyperactivity, idiopathic detrusor hyperacitivity, benign prostate hyperplasia (BPH-syndrome), lower urinary tract symptoms.

The compounds of the current invention are further suited for the treatment and/or prevention of conditions of pain, such as, e.g., menstrual disorders, dysmenorrhea, endometriosis, preterm delivery, tocolysis.

The compounds of the current invention are likewise suited for the treatment and/or prevention of erythematosis, onychomycosis, rheumatic diseases as well as for facilitation of wound healing.

The compounds of the current invention are also suited for the treatment and/or prevention of gastrointestinal diseases such as, e.g., diseases/disease states affecting the oesophagus, vomiting, achalasia, gastrooesophageal reflux disease, diseases of the stomach, such as, e.g., gastritis, diseases of the bowel, such as, e.g., diarrhea, constipation, malassimilation syndromes, syndromes of bile acid-loss, Crohn's Disease, Colitis ulcerosa, microscopic colitis, irritable bowel syndrome.

Furthermore, compounds of the current invention are suited for the treatment and/or prophylactic treatment of fibrotic diseases of inner organs such as lung, heart, kidney, bone marrow, and escpecially liver as well as dermatological fibrosis and fibrotic eye diseases. In the context of the current invention the term fibrotic diseases includes liver fibrosis, liver cirrhosis, lung fibrosis, endomyocardial fibrosis, cardiomyopathy, nephropathy, glomerulonephritis, interstitial kidney fibrosis, fibrotic damage as a consequence of diabetes, bone marrow fibrosis and similar fibrotic diseases, scleroderma, morphaea, keloids, hypertrophic scarring (also after surgical intervention), naevus, diabetic retinopathy and proliferative vitroretinopathy.

In addition, the compounds of the current invention can be used to treat and/or prophylactically treat dyslipidemias (hypercholesterolemia, hypertriglyceridemia, increased concentrations of post-prandial plasma triglycerides, hypo-alphalipoproteinemia, combined hyperlipidemias), metabolic diseases (type I and type II diabetes, metabolic syndrome, overweight, adip ositas), nepropathy and neuropathy, cancer (skin cancer, brain tumors, breast cancer, tumors of the bone marrow, leukemias, liposarcoma, carcinoma of the gastrointestinal tract, liver, pancreas, lung, kidney, ureter, prostate and gential tract as well as carcinoma of the lymphoproliferative system such as, e.g., Hodgkin's and Non-Hodgkin's lymphoma), of gastrointestinal and abdominal diseases (glossitis, gingivitis, periodontitis, esophagitis, eosinophilic gastroenteritis, mastocytosis, Crohn's disease, colitis, proctitis, anal pruritis, diarrhea, celiac disease, hepatitis, chronic hepatitis, liver fibrosis, liver zirrhosis, pancreatitis and cholecystitis), skin diseases (allergic skin diseases, psoriasis, acne, eczema, neurodermatitis, multiple kinds of dermatitis, as well as keratitis, bullosis, vasculitis, cellulitis, panniculitis, lupus erythematodes, erythema, lymphoma, skin cancer, Sweet-syndrome, Weber-Christian-syndrome, scarring, wart formation, chilblains), of diseases of the sceletal bones and the joints as well as of sceletal muscle (multiple kinds of arthritis, multiple kinds of arthropathies, scleroderma as well as of further diseases with inflammatory or immunologic components, such as, e.g., paraneoplastic syndrome, rejection reactions after organ transplantations and for wound healing and angiogenesis, especially with chronic wounds.

The compounds of the current invention are suited for the treatment and/or prophylactic treatment of ophthalmologic diseases such as, e.g., glaucoma, normotensive glaucoma, increased/high ocular pressure and their combination, of age-related macula degeneration (AMD), dry (non-exudative) AMD, wet (exudative, neovascular) AMD, choroidal neovascularization (CNV), retinal detachment, diabetic retinopathy, atrophic changes of the retinal pigmented epithelium (RPE), hypertrophic changes of the retinal pigmented epithelium, diabetic macula edema, diabetic retinopathy, retinal vein occlusion, choroidal retinal vein occlusion, macula edema, diabetic macula edema, macula edema as a consequence of retina vein occlusion, angiogenesis at the front-side of the eye such as corneal angiogenesis i.e. after keratitis, cornea transplantation or keratoplasty, corneal angiogenesis due to hypoxia (extensive wearing of contact lenses), Pterygium conjunctivae, sub-retinal edema and intra-retinal edema.

Furthermore, compounds of the current invention are suited for the treatment and/or prophylactic treatment of increased and high inner ocular pressure as a result of traumatic hyphema, periorbital edema, post-operative viscoelastic retention, intra-ocular inflammation, corticosteroid-use, pupil-block or idiopathic causes such as increased inner ocular pressure after trabeculectomy and due to pre-operative additives.

Furthermore, compounds of the current invention are suited for the treatment and/or prophylaxis of hepatitis, neoplasms, osteoporosis, glaucoma and gastroparesis.

Likewise, compounds of the current invention are suited for the regulation of cerebral blood circulation and represent useful agents for the treatment and or prophylaxis of migraine. T hey are also suited for the treatment and prophylaxis of cerebral infarcts such as stroke, cerebra ischemias and traumatic brain injury. Likewise, compounds of the current invention can be used for the treatment and/or prophylactic treatment of pain, neuralgias and tinnitus.

The aforementioned, well characterized human diseases may occur in other mammalians with a comparable etiology as well can be treated with the compounds of the current invention.

In the context of the current invention the term “treatment” or “treat” or “treated” includes inhibition, retardation, stopping, relief, reduction, attenuation, restriction, minimizing, suppression, repression or healing of a disease, a suffering, an illness, an injury or a health disorder, or the development, course and progression of such states/conditions and/or symptoms of these states/conditions.

The terms “prevention” and “prophylaxis” are used synonymously in the context of the present invention and refer to avoiding or reducing the risk to get, receive, experience, suffer from a disease, a suffering, an illness, an injury or a health disorder, the development, course and progression of such states/conditions and/or symptoms of these states/conditions.

Treatment or prevention of a disease, a suffering, an illness, an injury or a health disorder can be pursued in part or completely.

Other embodiments of the current invention therefore are directed to the use of the compounds of the current invention for the treatment and/or prevention of diseases, especially of the aforementioned diseases/disease states.

Other embodiments of the current invention therefore are directed to the use of the compounds of the current invention for the production/manufacturing of a compound/of compounds for the treatment and/or prevention of diseases, especially of the aforementioned diseases/disease states.

Other embodiments of the current invention are directed to a drug containing at least one of the compounds of the present invention for the treatment and/or prevention of diseases, especially of the aforementioned diseases/disease states.

Other embodiments of the current invention therefore are directed to the use of the compounds of the current invention in a process for the treatment and/or prevention of diseases, especially of the aforementioned diseases/disease states.

Other embodiments of the current invention are directed to a process for the treatment and/or prevention of diseases, especially of the aforementioned diseases, by use of an effective dose/amount of at least one of the compounds of the current invention.

Other embodiments of the current invention are directed to drugs and pharmaceutical formulations which contain at least one compound of the current invention, typically together with one or more inert, non-toxic, pharmaceutically suited additives, as well as their use for the aforementioned purposes.

Further embodiments of the current invention include the use of compound of formula (I) in a method for the treatment and/or prevention of heart diseases, vascular diseases, cardiovascular diseases, lung diseases, inflammatory diseases, fibrotic diseases, metabolic diseases and/or cardiometabolic diseases.

Further embodiments of the current invention include the use of compound of formula (I) in a method for the treatment and/or prevention of atherosclerosis, athersclerosis-related diseases such as coronary artery disease or peripheral vascular disease/arterialocclusive disease as well as post-surgery complications of these diseases such asrestenosis after angioplasty.

Further embodiments include the use of a compound according to formula (I) for the manufacture of a pharmaceutical composition for the treatment and/or preventin atherosclerosis, athersclerosis-related diseases such as coronary artery disease or peripheral vascular disease/arterial occlusive disease as well as post-surgery complications of these diseases such as restenosis after angioplasty.

Further embodiments include the use of a compound according to the current invention for the manufacture of a pharmaceutical composition for the treatment and/or prevention atherosclerosis, athersclerosis-related diseases such as coronary artery disease or peripheral vascular disease/arterial occlusive disease as well as post-surgery complications of these diseases such as restenosis after angioplasty.

Further embodiments include a pharmaceutical composition comprising a compound according to the current invention and one or more pharmaceutically acceptable excipients.

Other embodiments include a pharmaceutical composition comprising one or more first active ingredients, in particular compounds according to the current invention and one or more further active ingredients, in particular one or more additional therapeutic agents selected from the group consisting of angiotensin-converting enzyme inhibitors, angiotensin-receptor blockers, mineralocorticoid-receptor antagonists, endothelin antagonists, renin inhibitors, calcium blockers, beta-receptor blockers, vasopeptidase inhibitors, Sodium-Glucose-Transport-Antagonists, Metformin, Pioglitazones and Dipeptidyl-peptidase-IV inhibitors.

Further embodiments of the current invention include the pharmaceutical composition as defined above for the treatment and/or prevention of atherosclerosis, athersclerosis-related diseases such as coronary artery disease or peripheral vascular disease/arterial occlusive disease as well as post-surgery complications of these diseases such as restenosis after angioplasty.

Further embodiments of the invention include a method for the treatment and/or prevention atherosclerosis, athersclerosis-related diseases such as coronary artery disease or peripheral vascular disease/arterial occlusive disease as well as post-surgery complications of these diseases such as restenosis after angioplastyin a human or other mammal, comprising administering to a human or other mammal in need thereof a therapeutically effective amount of one or more compounds according to the current invention or of a pharmaceutical composition as defined above.

The compounds according to the invention can be used alone or in combination with one or more other pharmacologically active compounds if necessary as long as these combinations do not lead to unacceptable side effects. The present invention further relates to drugs containing at least one of the compounds according to the invention and one or more further active compounds/active components, in particular for the treatment and/or prophylaxis of the aforementioned diseases. As suitable active compounds/active components for combination, the following ones are mentioned exemplarily and preferably:
Positive inotropic compounds, such as, e.g., cardiac glycosides (digoxin), beta-adrenergic and dopaminergic agonists, such as isoprenaline, adrenaline, noradrenaline, dopamine or dobutamine and serelaxine;
Vasopressin-receptor antagonists, for example and preferably Conivaptan, Tolvaptan, Lixivaptan, Mozavaptan, Satavaptan, SR-121463, RWJ 676070 or BAY 86-8050, as well as the compounds described in WO 2010/105770, WO 2011/104322 and WO 2016/071212;
Natriuretic peptides, for example and preferably atrial natriuretic peptide (ANP), natriuretic peptide type B (BNP, Nesiritide), natriuretic peptide type C (CNP) or urodilatin;
Activators of cardiac myosin, for example and preferably e.g., Omecamtiv mecarbil (CK-1827452);
Calcium-sensitizers, for example and preferably Levosimendan;
Compounds affecting mitochondrial function and/or production of reactive oxygen species (ROS), for example Bendavia/Elamipritide;
Compounds influencing cardiac energy-metabolism, for example and preferably etomoxir, dichloroacetate, ranolazine, trimetazidine, full or partial adenosine A1 receptor agonists such as GS-9667 (formerly known as CVT-3619), capadenosone and neladenosone;
Compounds with an effect on heart rate, e.g., ivabradine
Organic nitrates and NO-donors, such as sodium nitroprusside, nitroglycerin, isosorbide mononitrate, isosorbide dinitrate, molsidomine or SIN-1, and inhalational NO;
Compounds that inhibit the degradation of cyclic guanosine monophosphate (cGMP) and/or cyclic adenosine monophosphate (cAMP), such as inhibitors of phosphodiesterases (PDE) 1, 2, 3, 4 and/or 5, in particular PDE 4 inhibitors such as roflumilast or revamilast and PDE 5 inhibitors such as sildenafil, vardenafil, tadalafil, udenafil, dasantafil, avanafil, mirodenafil, lodenafil or PF-00489791;
Compounds increasing cGMP synthesis, such as, e.g., sGC modulators in particular riociguat, nelociguat, vericiguat, cinaciguat and the compounds described in WO 00/06568, WO 00/06569, WO 02/42301, WO 03/095451, WO 2011/147809, WO 2012/004258, WO 2012/028647, WO 2012/059549, WO 2014/068099 and WO 2014/131760 as well as the compounds described in WO 01/19355, WO 01/19780, WO 2012/139888 and WO 2014/012934;
Compounds which inhibit the soluble epoxid-hydrolase (sEH), such as, e.g., N,N′-dicyclohexylurea, 12-(3-Adamantan-1-yl-ureido)-dodecanoic acid or 1-Adamantan-1-yl-3-{5-[2-(2-ethoxyethoxy)ethoxy]pentyl}-urea;
Compounds modulating neurotransmitters, such as, e.g., tricyclic antidepressants such as, e.g., amitryptiline and imaprimine, monoaminooxidase (MAO) inhibitors such as moclobemide, serotonin-noradrenaline-reuptake inibitors such as venlaflaxine, selective serotonin-reuptake inhibitors such as sertraline or noradrenergic and specific serotonergic antidepressants such as mirtazapine.
Compounds with anxiolytic, sedative and hypnotic properties, so-called tranquilizers such as, e.g., short- as well as mid-long acting benzodiazepines.
Prostacyclin analogs and IP receptor agonists, for example and preferably iloprost, bera-prost, treprostinil, epoprostenol, NS-304, selexipag, or ralinepag;
Compounds which inhibit the signal transduction cascade, in particular from the group of the tyrosine kinase inhibitors and/or from the group of serine/threoninekinase-inhibitors, for example and preferably dasatinib, nilotinib, bosutinib, regorafenib, sorafenib, sunitinib, cediranib, axitinib, telatinib, imatinib, brivanib, pazopanib, vatalanib, gefitinib, erlotinib, lapatinib, canertinib, lestaurtinib, pelitinib, semaxanib, masitinib or tandutinib, Rho kinase inhibitors, such as fasudil, Y-27632, SLx-2119, BF-66851, BF-66852, BF-66853, KI-23095 or BA-1049;
Anti-obstructive agents as used, for example, for the therapy of chronic-obstructive pulmonary disease (COPD) or bronchial asthma, for example and preferably inhalatively or systemically administered beta-receptor mimetics (e.g., bedoradrine) or inhalatively administered anti-muscarinergic substances;
Compounds with a bronchodilatory effect, for example and preferably from the group of β-adrenergic receptor-agonists, such as particularly albuterol, isoproterenol, metaproterenol, terbutaline, formoterol or salmeterol or from the group of anti-cholinergics, such as particularly ipratropiumbromide;
Anti-inflammatory and/or immunosuppressive agents as used, for example for the therapy of chronic-obstructive pulmonary disease (COPD), of bronchial asthma or pulmonary fibrosis, for example and preferably from the group of corticosteroids, such as particularly prednisone, prednisolone, methylprednisolone, triamcinolone, dexamethasone, beclomethasone, betamethasone, flunisolide, budesonide or fluticasone as well as from the group of non-steroidal anti-inflammatory drugs (NSAIDs), such as particularly acetylsalicylic acid (Aspirin), ibuprofen and naproxen, 5-aminosalicylic acid derivatives, leukotriene/leukotriene receptor antagonists,
TNF-α inhibitors and chemokine receptor antagonists, such as, e.g., CCR-1, -2 and/or -5 inhibitors. Furthermore, drugs such as pirfenidone, acetylcysteine, azathioprine or BIBF-1120;
Chemotherapeutics as used, for example, for the therapy of neoplasias of the lung or other organs;
Active compounds used for the systemic and/or inhalative treatment of pulmonary disorders, for example for cystic fibrosis (alpha-1-antitrypsin, aztreonam, ivacaftor, lumacaftor, ataluren, amikacin, levofloxacin), chronic obstructive pulmonary diseases (COPD) (LAS40464, PT003, SUN-101), acute respiratory distress syndrome (ARDS) and acute lung injury (ALI) (interferon-beta-1a, traumakines), obstructive sleep apnea (VI-0521), bronchiectasis (mannitol, ciprofloxacin), Bronchiolitis obliterans (cyclosporine, aztreonam) and sepsis (pagibaximab, Voluven, ART-123);
Active compounds used for treating muscular dystrophy, for example idebenone;
Antithrombotic agents, for example and preferably from the group of platelet aggregation inhibiting drugs (platelet aggregation inhibitors, thrombocyte aggregation inhibitors), anticoagulants or compounds with anticoagulant properties or profibrinolytic substances;
Active compounds for lowering blood pressure, for example and preferably from the group of calcium antagonists, angiotensin All antagonists, ACE inhibitors, NEP inhibitors, vasopeptidase inibitors, and combinations thereof, such as, e.g., sacubitril/valsartan (Entresto®), furthermore nicorandil, endothelin antagonists/endothelin receptor antagonists, such as bosentan, darusentan, ambrisentan, macicentan or sitaxentan, thromboxane A2 (TXA2) antagonists/thromboxane A2 (TBX2) receptor antagonists, renin inhibitors, alpha-receptor blockers, beta-receptor blockers, mineralocorticoid receptor antagonists, Rho-kinase inhibitors, diuretics as well as further vasoactive compounds/active components such as, e.g., adenosine and adenosine receptor agonists.
Compounds which inhibit degradation and remodelling of the extracellular matrix, for example and preferably inhibitors of matrix metalloproteases (MMPs), in particular chymase-inhibitors, stromelysin-inhibitors, collagenase-inhibitors, gelatinase-inhibitors and aggrecanase-inhibitors (in these terms especially MMP-1, MMP-3, MMP-8, MMP-9, MMP-10, MMP-11 aid MMP13) as well as inhibitors of the metallo-elastase MMP-12 as well as neutrophil elastase (HNE) inhibitors, for example and preferably sivelestat or DX-890 (Reltran);
Compounds which inhibit the binding of serotonin to its receptor, for example and preferably antagonists of the 5-HT₁-, 5-HT₂ₐ-, 5-HT_{2b}-, 5-HT_{2c}-, 5-HT₃- and 5-HT₄-receptors;

Anti-arrhythmic compounds/active components, for example and preferably sodium channel inhibitors, beta-receptor blockers, potassium channel blockers, calcium antagonists, I_{f}-channel blockers, digitalis, parasympatholytics (vagolytics), sympathomimetics and other anti-arrhythmic drugs such as, e.g., adenosine, adenosine-receptor agonists as well as vernakalant.

Active compounds that alter fat metabolism, for example and preferably from the group of thyroid receptor agonists, cholesterol synthesis inhibitors, for example and preferably HMG-CoA-reductase or squalene synthesis inhibitors, ACAT inhibitors, CETP inhibitors, MTP inhibitors, PPAR-alpha-, PPAR-gamma- and/or PPAR-delta-agonists, cholesterol absorption inhibitors, lipase inhibitors, polymeric bile acid adsorbers, bile acid reabsorption inhibitors aid lipoprotein(a) antagonists.

Active ingredients which inhibit neoangiogenesis, for example and preferably inhibitors of the VEGF and/or PDGF signalling pathways, inhibitors of the integrin signalling pathways, inhibitors of the angiopoietin-Tie signalling pathways, inhibitors of the PI3K-Akt-mTor signalling pathways, inhibitors of the Ras-Raf-Mek-Erk signalling pathway, inhibitors of the MAPK signalling pathways, inhibitors of the FGF signalling pathways, inhibitors of the sphingosine-1-phosphate signalling pathways, inhibitors of endothelial cell proliferation or apoptosis-inducing active ingredients;
Active ingredients which reduce vascular wall permeability (edema formation), for example and preferably corticosteroids, inhibitors of the ALK1-Smad1/5 signalling pathway, inhibitors of the VEGF and/or PDGF signalling pathways, cyclooxygenase inhibitors, inhibitors of the kallikrein-kinin system or inhibitors of the sphingosine-1-phosphate signalling pathways;
Active ingredients which reduce damage to the retina under oxidative stress, for example and perferably addressing the complement system, especially antagonists of the complement C5a receptor, or agonists of the 5-HT1A receptor;
Antioxidants and free-radical scavengers;
Active hypotensive ingredients, for example and preferably from the group of the calcium antagonists, angiotensin All antagonists, ACE inhibitors, beta-receptor blockers, alpha-receptor blockers, diuretics, phosphodiesterase inhibitors, sGC stimulators, cGMP elevators, aldosterone antagonists, mineralocorticoid receptor antagonists, ECE inhibitors and vasopeptidase inhibitors;
Antidiabetics and accordingly compounds/active components changing glucose metabolism, for example and preferably from the group of the insulins and/or insulin derivatives, biguanides, sulfonylureas, acarbose, DPP4-inhibitors, meglitinide derivatives, glucosidase inhibitors, GLP 1 receptor agonists/GLP1 analogues, SGLT-2 inhibitors, glucagon antagonists, PPAR-gamma agonists/insulin sensitizers, such as, e.g., thiazolidinediones, CCK1 receptor agonists, leptin receptor agonists, potassium channel antagonists and the inhibitors of hepatic enzymes that are involved in the stimulation of gluconeogenesis and/or glycogenolysis;
Antiinfectives, for example and in particular from the group of the antibacterial, antifungal and/or antiviral substances;
Substances for treatment of glaucoma, for example and in particular from the group of the adrenergics, beta-receptor blockers, carbonic anhydrase inhibitors, parasympathomimetics and prostaglandins;
Pain-reducing compounds such as opiates.

Antithrombotic agents are for example and preferably to be understood as compounds from the group of platelet aggregation inhibiting drugs (platelet aggregation inhibitors, thrombocyte aggregation inhibitors), anticoagulants or compounds with anticoagulant properties or profibrinolytic substances.

In preferred embodiments of the invention, the compounds according to the invention are administered in combination with compounds from the group of platelet aggregation inhibiting drugs (platelet aggregation inhibitors, thrombocyte aggregation inhibitors), for example and preferably aspirin, clopidogrel, prasugrel, ticlopidine, ticagrelor, cangrelor, elinogrel, tirofiban, PAR1-antagonists such as, e.g., vorapaxar, PAR4-antagonists, EP3-antagonists, such as, e.g., DG041 or inhibitors of adenosine-transport, such as dipyridamole;

In preferred embodiments of the invention, the compounds according to the invention are administered in combination with a thrombin inhibitor, for example and preferably ximelagatran, melagatran, dabigatran, bivalirudin or Clexane.

In preferred embodiments of the invention, the compounds according to the invention are administered in combination with a GPIIb/IIIa antagonist, for example and preferably tirofiban or abciximab.

In preferred embodiments of the invention, the compounds according to the invention are administered in combination with a factor Xa inhibitor, for example and preferably rivaroxaban, apixaban, edoxaban (DU-176b), darexaban, betrixaban, otamixaban, letaxaban, fidexaban, razaxaban, fondaparinux, idraparinux, as well as thrombin-inhibitors, for example and preferably dabigatran, dual thrombin/factor Xa-inhibitors, such as for example and preferably tanogitran or with factor XI- or factor XIa-inhibitors.

In preferred embodiments of the invention, the compounds according to the invention are administered in combination with heparin or a low molecular weight (LMW) heparin derivatives, such as, e.g., tinzaparin, certoparin, parnaparin, nadroparin, ardeparin, enoxaparin, reviparin, dalteparin, danaparoid, semuloparin (AVE 5026), adomiparin (M118) and EP-42675/ORG42675.

In preferred embodiments of the invention, the compounds according to the invention are administered in combination with a vitamin K antagonist, for example and preferably cou marines, such as marcumar/phenprocoumon.

In preferred embodiments of the invention, the compounds according to the invention are administered in combination with pro-fibrinolytic substances, for example and preferably streptokinase, urokinase or plasminogen-activator.

The agents for lowering blood pressure are preferably to be understood as compounds from the group of calcium antagonists, angiotensin All antagonists, ACE inhibitors, endothelin antagonists/endothelin receptor antagonists, thromboxane A2 (TBX2)-antagonists/thromboxane A2 (TBX2) receptor antagonists, renin inhibitors, alpha-receptor blockers, beta-receptor blockers, mineralocorticoid-receptor antagonists, Rho-kinase inhibitors as well as diuretics.

In preferred embodiments of the invention, the compounds according to the invention are administered in combination with a calcium antagonist, for example and preferably nifedipine, amlodipine, verapamil or diltiazem.

In preferred embodiments of the invention, the compounds according to the invention are administered in combination with an alpha-1-receptor blocker, for example and preferably prazosin.

In preferred embodiments of the invention, the compounds according to the invention are administered in combination with a beta-receptor blocker, for example and preferably propranolol, atenolol, timolol, pindolol, alprenolol, oxprenolol, penbutolol, bupranolol, metipranolol, nadolol, mepindolol, carazolol, sotalol, metoprolol, betaxolol, celiprolol, bisoprolol, carteolol, esmolol, labetalol, carvedilol, adaprolol, landiolol, nebivolol, epanolol or bucindolol.

In preferred embodiments of the invention, the compounds according to the invention are administered in combination with an angiotensin All antagonist, for example and preferably losartan, candesartan, valsartan, telmisartan or embursatan, irbesartan, olmesartan, eprosartan or azilsartan or a dual angiotensin All-antagonist/NEP-inhibitor, for example and preferably Entresto (LCZ696, Valsartan/Sacubitril).

In preferred embodiments of the invention, the compounds according to the invention are administered in combination with an ACE-inhibitor, for example and preferably enalapril, captopril, lisinopril, ramipril, delapril, fosinopril, quinopril, perindopril or tandopril.

In preferred embodiments of the invention, the compounds according to the invention are administered in combination with an endothelin antagonist/endothelin receptor antagonist, for example and preferably bosentan, darusentan, ambrisentan, avosentan, macicentan, atrasentan or sitaxsentan.

In preferred embodiments of the invention, the compounds according to the invention are administered in combination with a renin inhibitor, for example and preferably aliskiren, SPP-600 or SPP-800.

In preferred embodiments of the invention, the compounds according to the invention are administered in combination with a thromboxane A2 (TBX2)-antagonist, for example and preferably seratrodast or KP-496.

In preferred embodiments of the invention, the compounds according to the invention are administered in combination with a mineralocorticoid-receptor antagonist, for example and preferably spironolactone, eplerenone or finerenone.

In preferred embodiments of the invention, the compounds according to the invention are administered in combination with a diuretic, for example and preferably furosemide, torasemide bumetanide and piretanide, with potassium-saving diuretics, such as, e.g., amiloride or triamterene as well as with thiazide diuretics, such as, e.g., hydrochlorthiazide, chlorthalidone, xipamide and indapamide. Likewise, the combination with further diuretics is applicable, for example and preferably with bendroflumethiazide, chlorthiazide, hydroflumethiazide, methyclothiazide, polythiazide, trichlormethiazide, metolazone, quinethazone, acetazolamide, dichlorphenamide, methazolamide, glycerol, isosorbide or mannitol.

In preferred embodiments of the invention, the compounds according to the invention are administered in combination with a Rho-kinase inhibitor, for example and preferably fasudil, Y 27632, SLx-2119, BF-66851, BF-66852, BF-66853, KI-23095, SB-772077, GSK-269962A or BA-1049.

In preferred embodiments of the invention, the compounds according to the invention are administered in combination with natriuretic peptides, such as, for example “atrial natriuretic peptide” (ANP, Anaritide), “B-type natriuretic peptide”, “brain natriuretic peptide” (BNP, Nesiritide), “C-type natriuretic peptide” (CNP) or Urodilatin;

In preferred embodiments of the invention, the compounds according to the invention are administered in combination with inhibitors of the endopeptidase (NEP-inhibitors), for example Sacubitril, Omapatrilat orAVE-7688, or as dual combinations (‘ARNIs’) with Angiotensin receptor antagonists (for example Valsartan), such as, for example Entresto/LCZ696.

In preferred embodiments of the invention, the compounds according to the invention are administered in combination with type II antidiabetic drugs, such as inhibitors of the sodium-glucose co-transporter 2 (SGLT2 inhibitors), for example Empagliflozin, Canagliflozin, Dapagliflozin, Ipragliflozin, Tofogliflozin and inhibitors of the dipeptidyl peptidase 4 (DPP-4 inhibitors), for example sitagliptin, saxagliptin, linagliptin, alogliptin.

Substances altering fat metabolism are preferably to be understood as compounds from the group of CETP inhibitors, thyroid receptor agonists, cholesterol synthesis inhibitors such as HMG-CoA-reductase or squalene synthesis inhibitors, the ACAT inhibitors, MTP inhibitors, PPAR-alpha, PPAR-gamma and/or PPAR-delta agonists, cholesterol-absorption inhibitors, polymeric bile acid adsorbers, bile acid reabsorption inhibitors, lipase inhibitors as well as the lipoprotein(a) antagonists.

In preferred embodiments of the invention, the compounds according to the invention are administered in combination with a CETP inhibitor, for example and preferably torcetrapib (CP-529414), anacetrapib, JJT-705 or CET P-vaccine (Avant).

In preferred embodiments of the invention, the compounds according to the invention are administered in combination with a thyroid receptor agonist, for example and preferably D-thyroxin, 3,5,3′-triiodothyronin (T3), CGS 23425 or axitirome (CGS 26214).

In preferred embodiments of the invention, the compounds according to the invention are administered in combination with a HMG-CoA-reductase inhibitor from the class of statins, for example and preferably lovastatin, simvastatin, pravastatin, fluvastatin, atorvastatin, rosuvastatin or pitavastatin.

In preferred embodiments of the invention, the compounds according to the invention are administered in combination with a squalene synthesis inhibitor, for example and preferably BMS-188494 or TAK-475.

In preferred embodiments of the invention, the compounds according to the invention are administered in combination with an ACAT inhibitor, for example and preferably avasimibe, melinamide, pactimibe, eflucimibe or SMP-797.

In preferred embodiments of the invention, the compounds according to the invention are administered in combination with an MTP inhibitor, for example and preferably implitapide, BMS-201038, R-103757 or JTT-130.

In preferred embodiments of the invention, the compounds according to the invention are administered in combination with a PPAR-gamma agonist, for example and preferably pioglitazone or rosiglitazone.

In preferred embodiments of the invention, the compounds according to the invention are administered in combination with a PPAR-delta agonist, for example and preferably GW501516 or BAY 68-5042.

In preferred embodiments of the invention, the compounds according to the invention are administered in combination with a cholesterol-absorption inhibitor, for example and preferably ezetimibe, tiqueside or pamaqueside.

In preferred embodiments of the invention, the compounds according to the invention are administered in combination with a lipase inhibitor, for example and preferably orlistat.

In preferred embodiments of the invention, the compounds according to the invention are administered in combination with a polymeric bile acid adsorber, for example and preferably cholestyramine, colestipol, colesolvam, CholestaGel or colestimide.

In preferred embodiments of the invention, the compounds according to the invention are administered in combination with a bile acid reabsorption inhibitor, for example and preferably ASBT (=IBAT) inhibitors, such as, e.g., AZD-7806, S-8921, AK-105, BARI-1741, SC-435 or SC-635.

In preferred embodiments of the invention, the compounds according to the invention are administered in combination with a lipoprotein(a) antagonist, for example and preferably gemcabene calcium (CI-1027) or nicotinic acid.

Substances inhibiting signal transduction are preferably to be understood as compounds from the group of the tyrosine-kinase inhibitors and/or serine/threonine-kinase-inhibitors.

In preferred embodiments of the invention, the compounds according to the invention are administered in combination with a kinase-inhibitor, for example and preferably canertinib, erlotinib, gefitinib, dasatinib, imatinib, lapatinib, lestaurtinib, lonafarnib, nintedanib, nilotinib, bosutinib, axitinib, telatinib, brivanib, pazo¬panib, pegaptinib, peli¬tinib, semaxa¬nib, regora¬fenib, sora-fenib, sunitinib, tandutinib, tipifarnib, vatalanib, cediranib, masitinib, fasudil, lonidamine, leflunomide, BMS-3354825 or Y-27632.

Substances modulating glucose metabolism are preferably to be understood as compounds from the group of insulins, sulfonylureas, acarbose, DPP4-inhibitors, GLP-1 analogues or SGLT-2 inhibitors.

Substances modulating neurotransmitters are preferably to be understood as compounds from the group of tricyclic antidepressants, monoaminooxidase (MAO)-inhibitors, serotonin-noradrenaline-reuptake inhibitors (SNRI) and noradrenergic and specific serotonergic antidepressants (NaSSa).

In preferred embodiments of the invention, the compounds according to the invention are administered in combination with a tricyclic antidepressant, for example and preferably amitryptilin or imipramin.

In preferred embodiments of the invention, the compounds according to the invention are administered in combination with a monoaminooxidase (MAO)-inhibitor, for example and preferably moclobemide.

In preferred embodiments of the invention, the compounds according to the invention are administered in combination with a selective serotonine-noradrenaline reuptake inhibitor (SNRI), for example and preferably venlafaxine.

In preferred embodiments of the invention, the compounds according to the invention are administered in combination with a selective serotonine reuptake inhibitor (SSRI), such as, e.g., sertraline.

In preferred embodiments of the invention, the compounds according to the invention are administered in combination with a noradrenergic and specific serotonergic antidepressants (NaSSa), for example and preferably mirtazapine.

Substances with pain-reducing, anxiolytic or sedatative properties are preferably to be understood as compounds from the group of opiates and benzodiazepines.

In preferred embodiments of the invention, the compounds according to the invention are administered in combination with an opiate, for example and preferably morphine or sulfentanyl or fentanyl.

In preferred embodiments of the invention, the compounds according to the invention are administered in combination with a benzodiazepine, for example and preferably midazolam or diazepam.

Substances modulating cGMP-synthesis, such as, e.g., sGC-modulators, are preferably to be understood as compounds that stimulate or activate the soluble guanylate cyclase.

In preferred embodiments of the invention, the compounds according to the invention are administered in combination with sGC modulators, for example and preferably in riociguat, nelociguat, vericiguat, cinciguat and the compounds described in WO 00/06568, WO 00/06569, WO 02/42301, WO 03/095451, WO 2011/147809, WO 2012/004258, WO 2012/028647, WO 2012/059549, WO 2014/068099 and WO 2014/131760 as well as the compounds described in WO 01/19355, WO 01/19780, WO 2012/139888 and WO 2014/012934;

In preferred embodiments of the invention, the compounds according to the invention are administered in combination with full or partial adenosine A1 receptor agonists, such as, e.g., GS-9667 (formerly known as CVT-3619), capadenosone and neladenosone or compounds affecting mitochondrial function/ROS-production such as, e.g., Bendavia/elamipritide.

In preferred embodiments of the invention, the compounds according to the invention are administered in combination with a TGF-beta antagonist, for example and preferably pirfenidone or fresolimumab.

In preferred embodiments of the invention, the compounds according to the invention are administered in combination with a TNF-alpha antagonist, for example and preferably adalimumab.

In preferred embodiments of the invention, the compounds according to the invention are administered in combination with HIF-PH-inhibitors, for example and preferably molidustat or roxadustat.

In preferred embodiments of the invention, the compounds according to the invention are administered in combination with a serotonin-receptor antagonist, for example and preferably PRX-08066.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,

fillers and carriers (for example cellulose, microcrystalline cellulose (such as, for example, Avicel®), lactose, mannitol, starch, calcium phosphate (such as, for example, Di-Cafos®)),

ointment bases (for example petroleum jelly, paraffins, triglycerides, waxes, wool wax, wool wax alcohols, lanolin, hydrophilic ointment, polyethylene glycols),

bases for suppositories (for example polyethylene glycols, cacao butter, hard fat),

solvents (for example water, ethanol, isopropanol, glycerol, propylene glycol, medium chain-length triglycerides fatty oils, liquid polyethylene glycols, paraffins),

surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),

buffers, acids and bases (for example phosphates, carbonates, citric acid, acetic acid, hydrochloric acid, sodium hydroxide solution, ammonium carbonate, trometamol, triethanolamine),

isotonicity agents (for example glucose, sodium chloride),

adsorbents (for example highly-disperse silicas),

viscosity-increasing agents, gel formers, thickeners and/or binders (for example polyvinylpyrrolidone, methylcellulose, hydroxypropylmethylcellulose, hydroxypropyl cellulose, carboxymethylcellulose-sodium, starch, carbomers, polyacrylic acids (such as, for example, Carbopol®); alginates, gelatine),

disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),

flow regulators, lubricants, glidants and mould release agents (for example magnesium stearate, stearic acid, talc, highly-disperse silicas (such as, for example, Aerosil®)),

coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),

capsule materials (for example gelatine, hydroxypropylmethylcellulose),

synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),

plasticizers (for example polyethylene glycols, propylene glycol, glycerol, triacetine, triacetyl citrate, dibutyl phthalate),

penetration enhancers,

stabilisers (for example antioxidants such as, for example, ascorbic acid, ascorbyl palmitate, sodium ascorbate, butylhydroxyanisole, butylhydroxytoluene, propyl gallate),

preservatives (for example parabens, sorbic acid, thiomersal, benzalkonium chloride, chlorhexidine acetate, sodium benzoate),

colourants (for example inorganic pigments such as, for example, iron oxides, titanium dioxide),

flavourings, sweeteners, flavour- and/or odour-masking agents.

### Biological Definitions

Unless otherwise defined, all scientific and technical terms used in the description, figures aid claims have their ordinary meaning as commonly understood by one of ordinary skill in the art. All publications, patent applications, patents, and other references mentioned herein are incorporated by reference in their entirety. In case of conflict, the present specification, including definitions, will prevail. If two or more documents incorporated by reference include conflicting and/or inconsistent disclosure with respect to each other, then the document having the later effective date shall control. The materials, methods, and examples are illustrative only and not intended to be limiting. Unless stated otherwise, the following terms used in this document, including the description and claims, have the definitions given below.

The terms “comprising”, “including”, “containing”, “having” etc. shall be read expansively or open-ended and without limitation.

Singular forms such as “a”, “an” or “the” include plural references unless the context clearly indicates otherwise.

Unless otherwise indicated, the term “at least” preceding a series of elements is to be understood to refer to every element in the series. The terms “at least one” and “at least one of” include for example, one, two, three, four, or five or more elements.

Mutation numbering follows the species based positions for each construct, i.e. rat ADAMTS7 SP-Pro (1-217) followed by human ADAMTS7 CD (237-537).

The term “ADAMTS-7” (also ADAMTS7, ADAM-TS 7, ADAM-TS7) refers to the protein A disintegrin and metalloproteinase with thrombospondin motifs 7. The ADAMTS-7 protein is encoded by the gene ADAMTS-7. The ADAMTS-7 protein comprises human, murine, rat and further mammalian and non-mamalian homologues. Sequence(s) for human ADAMTS-7 are accessible via UniProt Identifier Q9UKP4 (ATS7_HUMAN), for instance human isoform Q9UKP4-1. Sequence(s) for murine ADAMTS-7 are accessible via UniProt Identifier Q68SA9 (ATS7_MOUSE). Different isoforms, variants and SNPs may exist for the different species aid are all comprised by the term ADAMTS-7. Also comprised are ADAMTS-7 molecules before and after maturation, i.e., independent of cleavage of one or more pro-domains. In addition, synthetic variants of the ADAMTS-7 protein may be generated and are comprised by the term ADAMTS-7. The protein ADAMTS-7 may furthermore be subject to various modifications, e.g, synthetic or naturally occurring modifications. Recombinant functional human ADAMTS-7 (e.g. according to SEQ ID No. 01 and 02) can be manufactured as described in the examples.

The term “ADAMTS-12” (also ADAMTS12, ADAM-TS 12, ADAM-TS12) refers to the protein A disintegrin and metalloproteinase with thrombospondin motifs 12. Such proteins preferably include a ADAMTS-12 catalytic domain. The ADAMTS-12 protein is encoded by the gene ADAMTS-12. The ADAMTS-12 protein comprises human, murine, rat and further mammalian and non-mamalian homologues. Sequence(s) for human ADAMTS-12 including the catalytic domains are accessible via UniProt Identifier P58397 (ATS12_HUMAN), for instance human isoform P58397-1. Sequence(s) for murine ADAMTS-12 are accessible via UniProt Identifier Q811 B3 (ATS12_MOUSE). Different isoforms and variants may exist for the different species and are all comprised by the term ADAMTS-12. Also comprised are ADAMTS-12 molecules before and after maturation, i.e., independent of cleavage of one or more pro-domains. In addition, synthetic variants of the ADAMTS-12 protein may be generated and are comprised by the term ADAMTS-12. The protein ADAMTS-12 may furthermore be subject to various modifications, e.g., synthetic or naturally occurring modifications. Recombinant functional human ADAMTS-12 (e.g., according to SEQ ID NO: 15) can be manufactured as described in the examples.

The terms “ADAMTS-4” and “ADAMTS-5” refer to the protein A disintegrin and metalloproteinase with thrombospondin motifs 4 and 5, respecitively. The ADAMTS-4 and -5 proteins are encoded by the genes ADAMTS4 and ADAMTS-5, respectively. These proteins comprises human, murine, rat and further mammalian and non-mamalian homologues. Sequence(s) for human ADAMTS-4/-5 are accessible via UniProt Identifier 075173 (ATS4_HUMAN)/Q9UNA0 (ATS5_HUMAN), respectively. Different isoforms and variants may exist. Recombinant active human ADAMTS-4 and ADAMTS-5 can be manufactured as known in the art.

The terms “MMP2”, “MMP12”, and “MMP15” refer to the 72 kDa type IV collagenase, Macrophage metalloelastase 2 and 12 and Matrix metalloproteinase-15, respectively. The MMP2, MMP12, and MMP15 proteins are encoded by the genes MMP2, MMP12, and MMP15, respectively. The proteins comprises human, murine, rat and further mammalian and non-mamalian homologues. Sequence(s) for human ADAMTS-4/-5 are accessible via UniProt Identifier P08253 (MMP2_HUMAN), P39900 (MMP12_HUMAN) and P51511 (MMP15_HUMAN), respectively. Different isoforms and variants may exist. Recombinant active human ADAMTS-4 and ADAMTS-5 can be manufactured as known in the art.

The term “ADAM17” refers to Disintegrin and metalloproteinase domain-containing protein 17, encoded by the gene ADAM17. The protein comprises human, murine, rat and further mammalian and non-mamalian homologues. Sequence(s) for human ADAM17 are accessible via UniProt Identifier P78536 (ADA17_HUMAN). Different isoforms and variants may exist. Recombinant active human ADAM17 can be manufactured as known in the art.

The term “prodomain” includes parts of ADAMTS-7 or ADAMTS-12 that are relatively N-terminal to the respective protein's functional chain (e.g., parts having metalloprotease function and disintergrin motifs). For example, a prodomain of ADAMTS-7 as provided in SEQ ID NO: 1 can include its signal peptide (residues 1-20) and its propeptide (residues 21-217), both of which are N-terminal to its peptidase domain, although not necessarily immediately N-terminal to it. In some embodiments, prodomain of ADAMTS-7 or ADAMTS-12 includes 75%, 80%, 85%, 90%, 95%, or 100% of the N-terminal part of the respective protein with its signal peptide plus its propeptide. The term “prodomain” also encompasses the parts of the encoded polypeptide that are processed (e.g., cleaved off) before generation of the functional enzymatic chain in the natural environment of the enzyme.

A “furin cleavage site” or furin consensus site is R-x-K/R-R↓D/S, cf. Shiryaev 2013 PLoS One. The ADAMTS7 prodomain contains multiple Furin protease cleavage sites, the last of which is thought to fully process the zymogen into the active form. Mutational analysis was described by Sommerville 2004 JBC for rat ADAMTS7 with R60A and R217A (referred to as mouse R220A in publication). R60A changes rat ADAMTS7 from LRKR↓D to LRKA↓D and R217A changes rat ADAMTS7 RQQR↓S to RQQA↓S.

The term “catalytic domain” includes parts of ADAMTS-7 or ADAMTS-12 that have ADAMTS-7 or ADAMTS-12 functionality, respectively, and that are C-terminal to the respective protein's prodomain. In some embodiments, the term “catalytic domain” refers to the peptidase plus disintegrin part of the respective protein (e.g., as characterized by UniProt), potentially also including any residues C-terminal to the respective protein's prodomain and N-terminal to the respective protein's peptidase domain. In some embodiments, the catalytic domain includes 75%, 80%, 85%, 90%, 95%, or 100% of the part of the respective enzyme having its disintegrin domain, its peptidase domain, and any residues it might have between its prodomain and its peptidase domain.

The term “functional protein” refers to a protein which has biological activity. For example, functional ADAMTS-7 refers to ADAMTS-7 which is able to catalyze the proteolytic cleavage of its (natural) substrate(s), e.g. TSP1 and/or COMP.

The term “metalloproteinase” refers to a protease enzyme whose catalytic mechanism involves a metal. Therefore a functional metalloproteinase is a functional protein, wherein the protein is a protease and wherein the protease is a metalloproteinase according to the foregoing definitions.

The expression “a cleavage site for a protease” refers to any peptide or protein sequence which is recognized and cleaved by the functional protease. A cleavage site for ADAMTS-7 thus refers to any peptide or protein sequence which is recognized and cleaved by functional ADAMTS-7. For example, being natural substrates of ADAMTS-7, the sequences of proteins COMP and TSP1 both comprise cleavage sites for ADAMTS-7. In particular the subsequence DELSSMVLELRGLRT (derived from TSP1, residues 275-289) constitutes or comprises a cleavage site for ADAMTS-7 and ADAMTS-12.

A “substrate” is a molecule upon which an enzyme acts. For example, the substrate of a proteinase can be a peptide or protein or derivative thereof, which is cleaved by the proteinase. Metalloproteinase paralogs ADAMTS-7/-12 on the one hand and their common peptide substrate on the other hand are interrelated in the sense of a plug and socket relationship.

The term “COMP”, TSP-5 or TSP5 refers to the protein Cartilage oligomeric matrix protein. The COMP protein is encoded by the gene COMP. The COMP protein comprises human, murine, rat and further mammalian and homologues. Sequence(s) for human COMP are accessible via UniProt Identifier P49747 (COMP_HUMAN), for instance human isoform P49747-1. Sequence(s) for murine COMP are accessible via UniProt Identifier Q9ROG6 (COMP_MOUSE). Different isoforms and variants may exist for the different species and are all comprised by the term COMP. Also comprised are COMP molecules before and after maturation, i.e., independent of cleavage of one or more pro-domains. In addition, synthetic variants of the COMP protein may be generated and are comprised by the term COMP. The protein COMP may furthermore be subject to various modifications, e.g, synthetic or naturally occurring modifications. Recombinant human COMP or derivatives thereof can be manufactured as described in the examples.

The term “TSP1” (also THBS1 or TSP) refers to the protein Thrombospondin-1. The TSP1 protein is encoded by the gene THBS1. The TSP1 protein comprises human, murine, rat and further mammalian and non-mammalian homologues. Sequence(s) for human TSP1 are accessible via UniProt Identifier P07996 (TSP1_HUMAN), for instance human isoform P07996-1. Sequence(s) for murine TSP1 are accessible via UniProt Identifier P35441 (TSP1_MOUSE). Different isoforms and variants may exist for the different species and are all comprised by the term TSP1. Also comprised are TSP1 molecules before and after maturation, i.e., independent of cleavage of one or more pro-domains. In addition, synthetic variants of the TSP1 protein may be generated and are comprised by the term TSP1. The protein TSP1 may furthermore be subject to various modifications, e.g, synthetic or naturally occurring modifications. Recombinant human TSP1 or derivatives thereof can be manufactured as described in the examples.

The term “fluorophore” according to the current invention refers to a molecule or chemical group which has the ability to absorb energy from light, transfer this energy internally, and emit this energy as light of a characteristic wavelength. Without being bound by theory, since some energy is lost during this process, the energy of the emitted fluorescence light is lower than the energy of the absorbed light, and therefore emission occurs at a longer wavelength than absorption. A variety of fluorophores and quenchers has been described in the art and can be used according to the current invention, see for example Bajar et al, Sensors 2016, A Guide to Fluorescent Protein FRET Pairs.

The term “quencher” according to the current invention refers to a molecule or chemical group which has the ability to decrease the fluorescence intensity of a given fluorophore. Without being bound by theory a variety of processes can result in quenching, such as excited state reactions, energy transfer, complex-formation and collisional quenching. Dark quenchers are dyes with no native fluorescence. Quencher fluorescence can increase background noise due to overlap between the quencher and reporter fluorescence spectra. A variety of fluorophores aid quenchers has been described in the art and can be used according to the current invention, see for example Bajar et al, Sensors 2016, A Guide to Fluorescent Protein FRET Pairs. Suitable quenchers according to the current invention include DDQ-I A (430 nm), Dabcyl (475 nm), Eclipse B (530 nm), Iowa Black FQ C (532 nm), BHQ-1D (534 nm), QSY-7 E (571 nm), BHQ-2 D (580 nm), DDQ-II A (630 nm), Iowa Black RQ C (645 nm), QSY-21 E (660 nm) or BHQ-3 D (670 nm). Preferred examples of quenchers according to the current invention include Dabsyl (dimethylaminoazobenzenesulfonic acid), which absorbs in the green spectrum and is often used with fluorescein, black hole quenchers which are capable of quenching across the entire visible spectrum, Qxl quenchers which span the full visible spectrum, Iowa black FQ (absorbs in the green-yellow part of the spectrum), Iowa black RQ (blocks in the orange-red part of the spectrum) and IRDye QC-1 (quenches dyes from the visible to the near-infrared range (500-900 nm)).

Suitable internally quenched fluorescent (IQF)/FRET pairs include ABz-Tyr(NO2), ABz-EDDNP, Trp-Dansyl, and 7-methoxy-coumarin-4-yl acetic acid-2,4-dinitrophenyl-lysine (MCA-Lys(DNP)) (Poreba, Marcin et al., Highly sensitive and adaptable fluorescence-quenched pair discloses the substrate specificity profiles in diverse protease families. Scientific reports 7, 43135. (2017), doi:10.1038/srep43135). Further suitable examples are listed in: Poreba M. & Drag M. Current strategies for probing substrate specificity of proteases. Curr Med Chem 17, 3968-3995 (2010).

The term “construct” refers to nucleic acids (e.g., double stranded DNA, which can be in the form of a plasmid).

The term “align” in the context of two sequences (e.g., amino acid sequences), includes arranging the two sequences with respect to each other (e.g., the first sequence along a first row, and the second sequence along a second row, potentially with g ap(s) in one or both sequences) to obtain a measure of their relationship to each other (e.g., % identity, % similarity, alignment score). A particular alignment of two sequences can be optimal (i.e., there are no alignments of the two sequences that result in a higher alignment score, if alignment score is the metric of concern for optimality) or non-optimal. In addition, a particular alignment can be global or local with respect to either sequence. For example, if the alignment is global with respect to both sequences (i.e., a global-global alignment), then all residues of both of the sequences are factored in to the calculation of the measure of their relationship, including any internal or external gaps in either sequence. In contrast, in a global-local alignment, while all residues and gaps in the first sequence are considered, no external gaps in the second sequence are considered, thereby allowing fitting the first sequence into a part of the second sequence.

The term “Needleman-Wunsch score” implies that either a global-global or a global-local (or local-global) alignment has been used to generate the alignment score. When partiality modifiers are used for both the first sequence (e.g., “portion”) and the second sequence (e.g., “segment”), this implies that the alignment is global-global. A “Needleman-Wunsch score” includes scores calculated by the originally published method (S. B. Needleman & C. D. Wunsch, A genera method applicable to the search for similarities in the amino acid sequence of two prote ins, J. Mol. Biol. 48(3):443-53 (1970)) as well as by subsequent refinements of the method. Although Needleman-Wunsch algorithm is able to, and is designed to, find an optimal alignment, and thereby a maximum alignment score, the term “Needleman-Wunsch score” as used here is not restricted to the optimal/maximum score; it can be the alignment score of any alignment of the two sequences as long as at least one of the sequences (e.g., as defined, for example as a residue range) is considered globally.

Alignment methods (e.g., Needleman-Wunsch) that generate an alignment score (e.g., Needleman-Wunsch score) can make use of a substitution matrix. A particular substitution matrix that can be used is BLOSUM62, which is reproduced below in Table B1.

TABLE B1

| | A | R | N | D | C | Q | E | G | H | I | L | K | M | F | P | S | T | W | Y | V | B | Z | X | * |
|---|---|---|---|---|---|---|---|---|---|---|---|---|---|---|---|---|---|---|---|---|---|---|---|---|
| A | 4 | −1 | −2 | −2 | 0 | −1 | −1 | 0 | −2 | −1 | −1 | −1 | −1 | −2 | −1 | 1 | 0 | −3 | −2 | 0 | −2 | −1 | 0 | −4 |
| R | −1 | 5 | 0 | −2 | −3 | 1 | 0 | −2 | 0 | −3 | −2 | 2 | −1 | −3 | −2 | −1 | −1 | −3 | −2 | −3 | −1 | 0 | −1 | −4 |
| N | −2 | 0 | 6 | 1 | −3 | 0 | 0 | 0 | 1 | −3 | −3 | 0 | −2 | −3 | −2 | 1 | 0 | −4 | −2 | −3 | 3 | 0 | −1 | −4 |
| D | −2 | −2 | 1 | 6 | −3 | 0 | 2 | −1 | −1 | −3 | −4 | −1 | −3 | −3 | −1 | 0 | −1 | −4 | −3 | −3 | 4 | 1 | −1 | −4 |
| C | 0 | −3 | −3 | −3 | 9 | −3 | −4 | −3 | −3 | −1 | −1 | −3 | −1 | −2 | −3 | −1 | −1 | −2 | −2 | −1 | −3 | −3 | −2 | −4 |
| Q | −1 | 1 | 0 | 0 | −3 | 5 | 2 | −2 | 0 | −3 | −2 | 1 | 0 | −3 | −1 | 0 | −1 | −2 | −1 | −2 | 0 | 3 | −1 | −4 |
| E | −1 | 0 | 0 | 2 | −4 | 2 | 5 | −2 | 0 | −3 | −3 | 1 | −2 | −3 | −1 | 0 | −1 | −3 | −2 | −2 | 1 | 4 | −1 | −4 |
| G | 0 | −2 | 0 | −1 | −3 | −2 | −2 | 6 | −2 | −4 | −4 | −2 | −3 | −3 | −2 | 0 | −2 | −2 | −3 | −3 | −1 | −2 | −1 | −4 |
| H | −2 | 0 | 1 | −1 | −3 | 0 | 0 | −2 | 8 | −3 | −3 | −1 | −2 | −1 | −2 | −1 | −2 | −2 | 2 | −3 | 0 | 0 | −1 | −4 |
| I | −1 | −3 | −3 | −3 | −1 | −3 | −3 | −4 | −3 | 4 | 2 | −3 | 1 | 0 | −3 | −2 | −1 | −3 | −1 | 3 | −3 | −3 | −1 | −4 |
| L | −1 | −2 | −3 | −4 | −1 | −2 | −3 | −4 | −3 | 2 | 4 | −2 | 2 | 0 | −3 | −2 | −1 | −2 | −1 | 1 | −4 | −3 | −1 | −4 |
| K | −1 | 2 | 0 | −1 | −3 | 1 | 1 | −2 | −1 | −3 | −2 | 5 | −1 | −3 | −1 | 0 | −1 | −3 | −2 | −2 | 0 | 1 | −1 | −4 |
| M | −1 | −1 | −2 | −3 | −1 | 0 | −2 | −3 | −2 | 1 | 2 | −1 | 5 | 0 | −2 | −1 | −1 | −1 | −1 | 1 | −3 | −1 | −1 | −4 |
| F | −2 | −3 | −3 | −3 | −2 | −3 | −3 | −3 | −1 | 0 | 0 | −3 | 0 | 6 | −4 | −2 | −2 | 1 | 3 | −1 | −3 | −3 | −1 | −4 |
| P | −1 | −2 | −2 | −1 | −3 | −1 | −1 | −2 | −2 | −3 | −3 | −1 | −2 | −4 | 1 | −1 | −1 | −4 | −3 | −2 | −2 | −1 | −2 | −4 |
| S | 1 | −1 | 1 | 0 | −1 | 0 | 0 | 0 | −1 | −2 | −2 | 0 | −1 | −2 | −1 | 4 | 1 | −3 | −2 | −2 | 0 | 0 | 0 | −4 |
| T | 0 | −1 | 0 | −1 | −1 | −1 | −1 | −2 | −2 | −1 | −1 | −1 | −1 | −2 | −1 | 1 | 5 | −2 | −2 | 0 | −1 | −1 | 0 | −4 |
| W | −3 | −3 | −4 | −4 | −2 | −2 | −3 | −2 | −2 | −3 | −2 | −3 | −1 | 1 | −4 | −3 | −2 | 11 | 2 | −3 | −4 | −3 | −2 | −4 |
| Y | −2 | −2 | −2 | −3 | −2 | −1 | −2 | −3 | 2 | −1 | −1 | −2 | −1 | 3 | −3 | −2 | −2 | 2 | 7 | −1 | −3 | −2 | −1 | −4 |
| V | 0 | −3 | −3 | −3 | −1 | −2 | −2 | −3 | −3 | 3 | 1 | −2 | 1 | −1 | −2 | −2 | 0 | −3 | −1 | 4 | −3 | −2 | −1 | −4 |
| B | −2 | −1 | 3 | 4 | −3 | 0 | 1 | −1 | 0 | −3 | −4 | 0 | −3 | −3 | −2 | 0 | −1 | −4 | −3 | −3 | 4 | 1 | −1 | −4 |
| Z | −1 | 0 | 0 | 1 | −3 | 3 | 4 | −2 | 0 | −3 | −3 | 1 | −1 | −3 | −1 | 0 | −1 | −3 | −2 | −2 | 1 | 4 | −1 | −4 |
| X | 0 | −1 | −1 | −1 | −2 | −1 | −1 | −1 | −1 | −1 | −1 | −1 | −1 | −1 | −2 | 0 | 0 | −2 | −1 | −1 | −1 | −1 | −1 | −4 |
| * | −4 | −4 | −4 | −4 | −4 | −4 | −4 | −4 | −4 | −4 | −4 | −4 | −4 | −4 | −4 | −4 | −4 | −4 | −4 | −4 | −4 | −4 | −4 | 1 |

In this table, the first 20 columns (and the first 20 rows) labelled with single-letter amino acid codes represent the standard amino acids. The remaining columns/rows represent additional residue types (e.g., “B” for Asx (Asn/Asp), “Z” for Glx (Gln/Glu), “X” for any amino acid, and “*” for a translation stop encoded by a termination codon). If the sequences of interest have only a subset of these residues, then a corresponding sub-matrix of BLOSUM62 can also be alternatively sufficient for aligning them (e.g., the scores from the upper-left 20×20 part of the scores if only standard amino acids that are singly-identified are of concern).

As a demonstration of using BLOSUM62 in calculating an alignment score, if residues 1-8 of SEQ ID NO: 14 (“EEVKAKVQ”) were aligned with themselves, the alignment score would be the sum of scores for each of those amino acids pairing with itself (e.g., when residue “A” in the first sequence pairs with itself, an “A,” in the second sequence, it contributes a score of 4, as seen in the cell at row 2, column 2 of the BLOSUM62 matrix). Therefore, the alignment score in this hypothetical example would be 37 (5+5+4+5+4+5+4+5). If the “A” in the second sequence, in this hypothetical, is changed into a “W,” the alignment score would be 30 (5+5+4+5-3+5+4+5). If the “E” at residue one, or the “K” at residue four, or the “Q” at residue eight is deleted in the second sequence instead, in this hypothetical, the alignment score in each case would be 20 (the original total score of 37 is lessened by 5 due to the deleted residue, and there is an additional total gap penalty of 12 in this case, as explained further next). The total gap penalty is calculated by summing a gap extension penalty as multiplied by the number of residues in the gap with a gap-opening penalty. As an example, a gap-opening penalty of 11 and a gap extension penalty of 1 can be used. In that case, a single gap as in the last hypothetical example would result in a total gap penalty of 12 (11+1*1), regardless of whether the gap is internal or external, which can be the case for global-global alignments. If the gap were three amino acids long in the middle of a sequence, the total gap penalty would be 14 (11+1*3), which would be subtracted from of the scores of the aligning residues (identical as well as non-identical) to arrive at the alignment score.

Using a substitution matrix such as BLOSUM62 is superior to cruder methods such as percent identity calculations, at least because different aligned identical residues can give different contributions to the overall score depending on how rare or common themselves or their mutations are (e.g., a W:W alignment contributes 11, as seen in Table B1, while an A:A alignment contributes only 4). In addition, mutations into different residues can also be treated differently with a substitution matrix (e.g., a D:E change has a positive score, 2, as seen in Table B1, while a D:L change has a negative score, −4, whereas each of these changes would be clumped as the same non-identical change in a percent-identity approach). Overall, using a substitution matrix like BLOSUM62 provides a dramatically more sensitive measure for inferring sequence relatedness than percent identity methods, since the matrix allows calibrating the score of changing each of the amino acids (e.g., 20) into each of the amino acids (e.g., 20) individually, while with percent identity the changes are limited to merely identical and non-identical ones. As a result, a polypeptide sequence defined with respect to its alignment to a reference sequence in terms of a Needleman-Wunsch score obtained by using BLOSUM62 matrix is significantly more likely to have structural features, physical properties, and functional features in common with the polypeptide of the reference sequence.

For percent sequence identity values, the same alignment method (e.g., Needleman-Wunsch algorithm for global-global alignment, using BLOSUM62 matrix, with gap opening penalty of 11 and a gap extension penalty of 1) can be used to obtain the alignment, after which the pairs of aligned identical residues can be counted and then divided by the total length of the alignment (including gaps, internal as well as external) to arrive at the percent identity value. For percent similarity values, the same approach as for percent identity values can be used, except that what is counted, instead of pairs of identical residues, would be the aligned residue pairs with BLOSUM62 values that are not negative (i.e.,

Numerous programs as well as websites exist for calculation of alignment scores. For example, executables for local use can be downloaded from the UVa FASTAServer (available from World Wide Web at fasta.bioch.virginia.edu/fasta_www2/fasta_list2.shtml). Among the programs available at the UVa FASTA server, ggsearch can perform global-global alignments (e.g., using Needleman-Wunsch algorithm, and BLOSUM62 matrix with gap opening penalty of 11 and a gap extension penalty of 1) and glsearch can perform global-local alignments. Similar functionality is also available through an online submission form at the same server (e.g., World Wide Web at fasta.bioch.virginia.edu/fasta_www2/fasta_www.cgi?rm=select&pgm=gnw, after selecting “Align two sequences” to align two sequences rather than one sequence against a database). Additional online sources for similar functionality include the National Center for Biotechnology Information (available through the World Wide Web at https://blast.ncbi.nlm.nih.gov/Blast.cgi) and the European Bioinformatics Institute of the European Molecular Biology Laboratory (available through the World Wide Web at https://www.ebi.ac.uk/Tools/sss/fasta/).

### BIOLOGICAL EMBODIMENTS

According to a first biological aspect of the current invention, there is provided a recombinant nucleic acid sequence for the improved expression and purification of functional ADAMTS-7. According to some first embodiments according to the first biological aspect, there is provided a recombinant nucleic acid for expression of an ADAMTS-7 polypeptide that comprises a rodent prodomain of ADAMTS-7 as a first portion and a functional human ADAMTS-7 as a second portion.

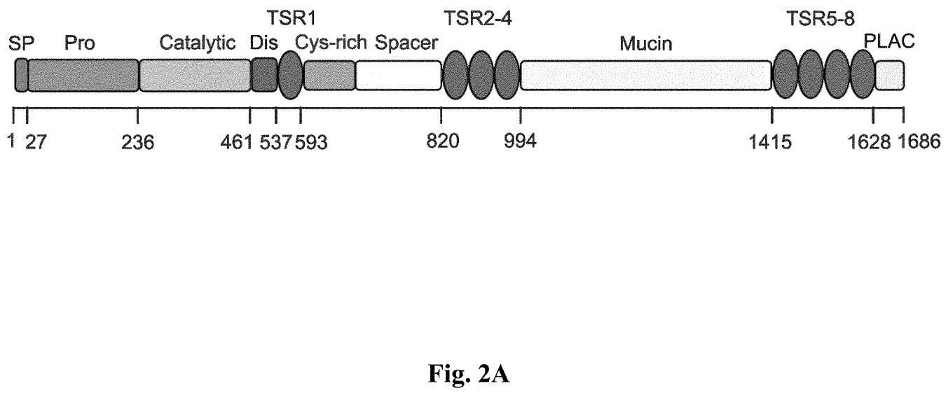

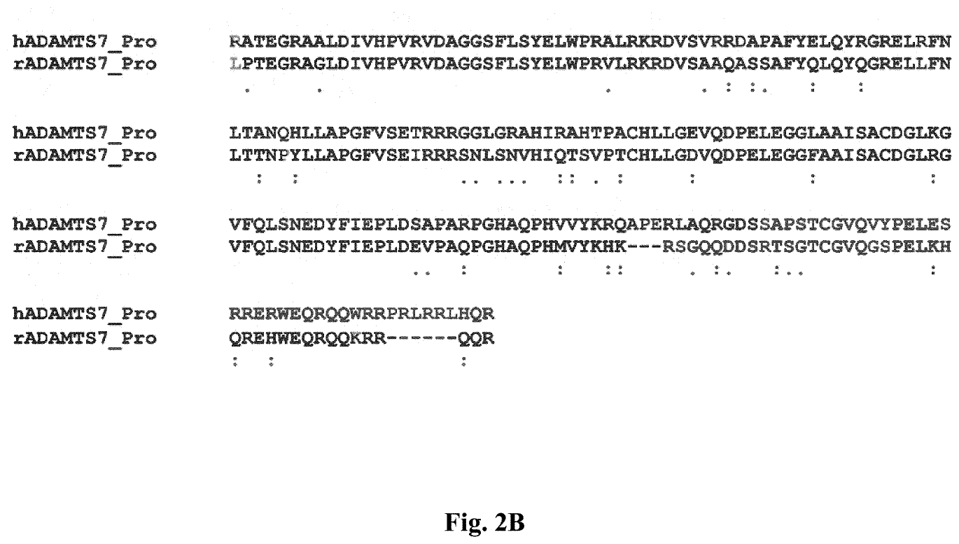

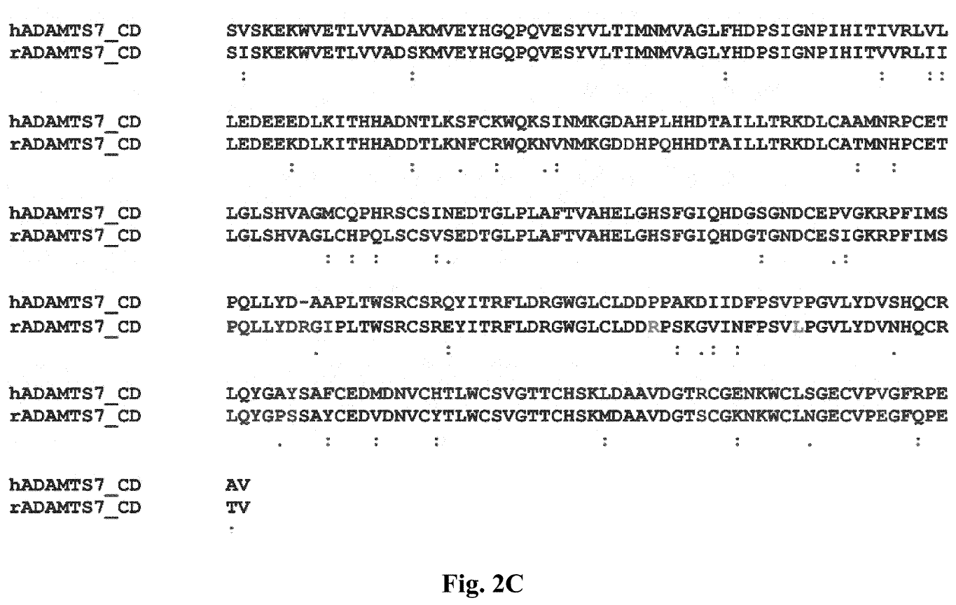

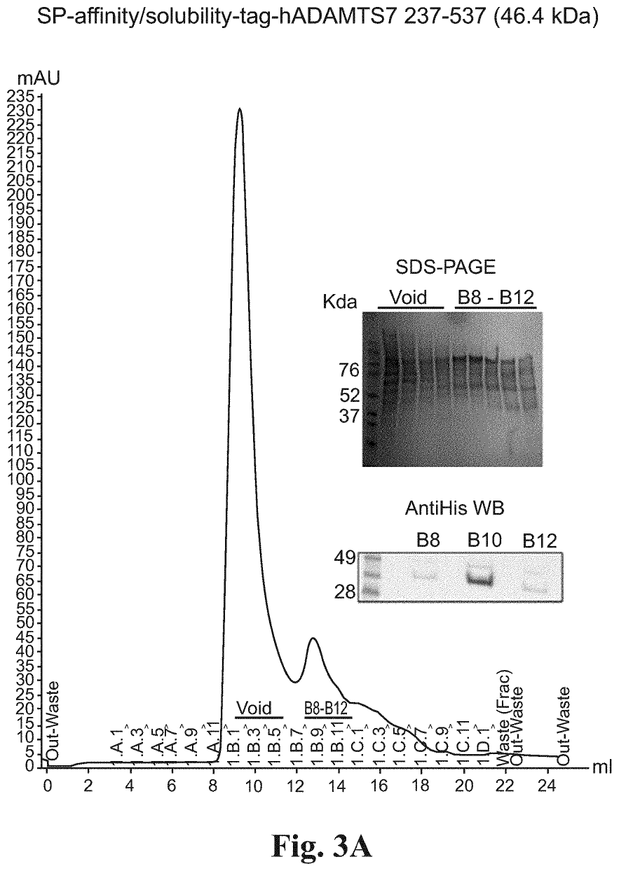

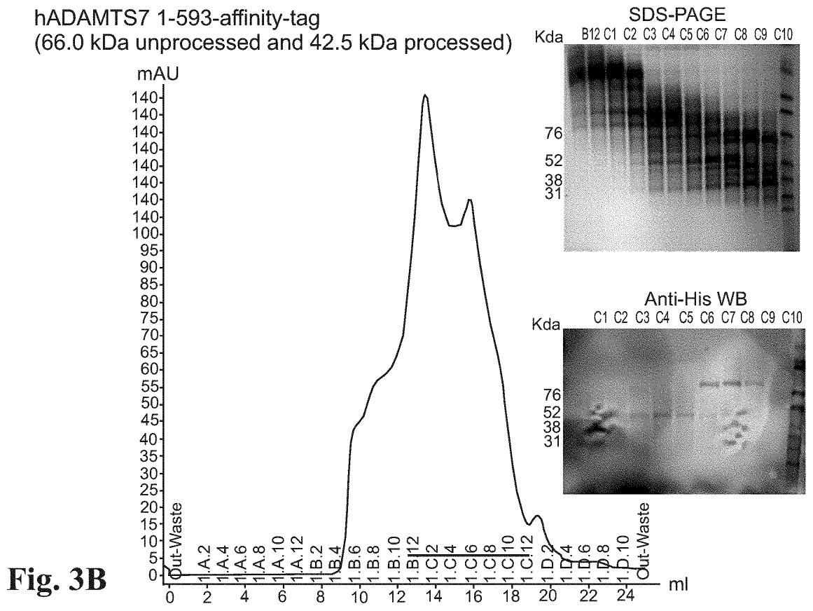

Commercial expression and purification of recombinant functional ADAMTS-7 has so far been a challenge (cf. FIG. 3), rendering the development of an assay for the identification and characterization of modulators of ADAMTS-7 extremely difficult. For example, as shown in FIG. 3, expression of hADAMTS-7 (residues 237-537) or hADAMTS-7 Pro-CD-TSR1 (residues 1-593) yielded little soluble proteins. The nucleic acids according to the first biological aspect solved the problem of protein solubility as well as expression yield, both of which were lower for fully human ADAMTS-7 constructs compared to hybrid constructs (cf. example B2).

The constructs according to the first biological aspect are suited for the production of sufficient amounts of ADAMTS-7 with reproducible activity and purity. The resulting recombinant functional ADAMTS-7 can therefore be used in an assay for the identification and characterization of modulators of ADAMTS-7.

In order to obtain a suitable construct for the expression of functional ADAMTS-7, more than 50 E. coli constructs, 20 constructs from Baculovirus and 30 HEK293 constructs were designed aid evaluated to achieve the efficient expression of functional ADAMTS-7. The recombinant nucleic acid according to the first biological aspect surprisingly solved these problems and enabled the efficient expression and purification of functional ADAMTS-7 protein: In particular it was surprisingly found that a rodent prodomain is more effective in driving folding of the catalytic domain of human ADAMTS-7, thereby improving the yield of the soluble ADAMTS-7 proteins ˜10 fold (see example B2).

In some embodiments according to the first biological aspect, the recombinant nucleic acid sequence encodes for a recombinant polypeptide that comprises a first portion and a second portion, wherein the first portion has a sequence identity of >80% with the sequence of residues 1-217 of SEQ ID NO: 1 or with the sequence of residues 1-217 of SEQ ID NO: 2, and the second portion has a sequence identity of >80% with the sequence of residues 218-518 of SEQ ID NO: 1.

In some embodiments according to the first biological aspect, the recombinant nucleic acid sequence encodes for a recombinant polypeptide that comprises a first portion and a second portion, wherein the first portion has a sequence identity of >90% with the sequence of residues 1-217 of SEQ ID NO: 1 or with the sequence of residues 1-217 of SEQ ID NO: 2, and the second portion has a sequence identity of >90% with the sequence of residues 218-518 of SEQ ID NO: 1.

In some embodiments according to the first biological aspect, the recombinant nucleic acid sequence encodes for a recombinant polypeptide that comprises a first portion and a second portion, wherein the first portion has a sequence identity of >95% with the sequence of residues 1-217 of SEQ ID NO: 1 or with the sequence of residues 1-217 of SEQ ID NO: 2, and the second portion has a sequence identity of >95% with the sequence of residues 218-518 of SEQ ID NO: 1.

In some embodiments according to the first biological aspect, the recombinant nucleic acid sequence encodes for a recombinant polypeptide that comprises a first portion and a second portion, wherein the first portion has a sequence identity of >98% with the sequence of residues 1-217 of SEQ ID NO: 1 or with the sequence of residues 1-217 of SEQ ID NO: 2, and the second portion has a sequence identity of >98% with the sequence of residues 218-518 of SEQ ID NO: 1.

In some embodiments according to the first biological aspect, the recombinant nucleic acid sequence encodes fora recombinant polypeptide that comprises a first portion and a second portion, wherein the first portion comprises residues 1-217 of SEQ ID NO: 1 or residues 1-217 of SEQ ID NO: 2, and/or the second portion comprises residues 218-518 of SEQ ID NO: 1 (cf. example B1). In some embodiments the second portion is C-terminal to the first portion.

In some embodiments according to the first biological aspect, the recombinant nucleic acid sequence encodes for a polypeptide that has a first portion and a second portion. The first portion of the polypeptide, in some embodiments, has an amino acid sequence that when aligned with an amino acid sequence of a ADAMTS-7 prodomain or a fragment thereof from a first species generates a Needleman-Wunsch score greater than 700, if BLOSUM62 matrix, a gap opening penalty of 11, and a gap extension penalty of 1 are used. In some embodiments, this generated Needleman-Wunsch score is greater than 750, 800, 850, 900, 950, 1000, 1050, or 1100. In some embodiments, the first portion and the ADAMTS-7 prodomain share a sequence identity that is at least 70%, 75%, 80%, 85%, 90%, 95%, 97% or 98%. The second portion of the polypeptide, in some embodiments, has an amino acid sequence that when aligned with an amino acid sequence of a ADAMTS-7 catalytic domain or a fragment thereof from a second species generates a Needleman-Wunsch score greater than 1000 if BLOSUM62 matrix, a gap opening penalty of 11, and a gap extension penalty of 1 are used. In some embodiments, this generated Needleman-Wunsch score is greater than 1050, 1100, 1150, 1200, 1250, 1300, 1350, 1400, 1450, 1500, 1550, 1600, or 1650. In some embodiments, the second portion and the ADAMTS-7 catalytic domain share a sequence identity that is at least 70%, 75%, 80%, 85%, 90%, 95%, 97% or 98%.

In some embodiments according to the first biological aspect, the recombinant nucleic acid sequence encodes for a recombinant polypeptide that comprises a first portion having an amino acid sequence that aligns with an amino acid sequence of an ADAMTS-7 prodomain or a fragment thereof from a first species with a Needleman-Wunsch score greater than 700, when BLOSUM62 matrix, a gap opening penalty of 11, and a gap extension penalty of 1 are used; and a second portion having an amino acid sequence that aligns with an amino acid sequence of a ADAMTS-7 catalytic domain or a fragment thereof from a second species with a Needleman-Wunsch score greater than 1000 when BLOSUM62 matrix, a gap opening penalty of 11, and a gap extension penalty of 1 are used.

In some embodiments, the first species is a non-human species, such as a rodent species, such as rat. In some different or the same embodiments the second species is human, e.g. the catalytic domain is derived from human ADAMTS-7. In some embodiments the first species is a rodent species and the second species is human.

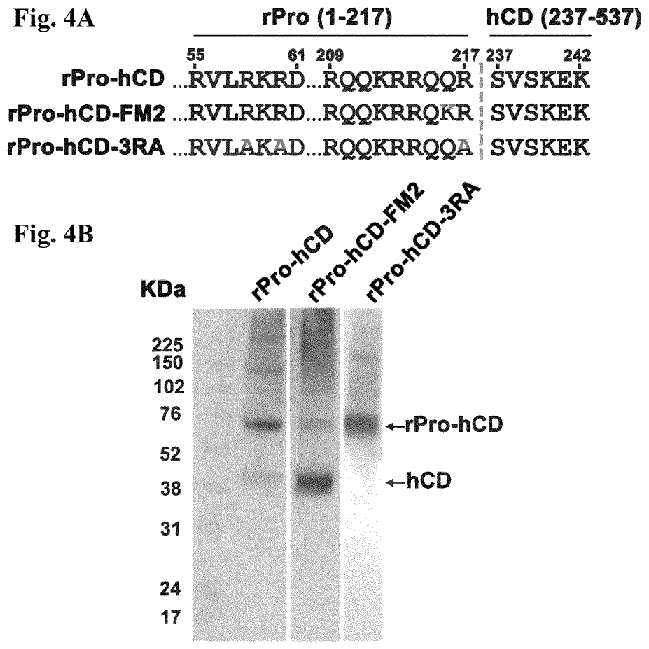

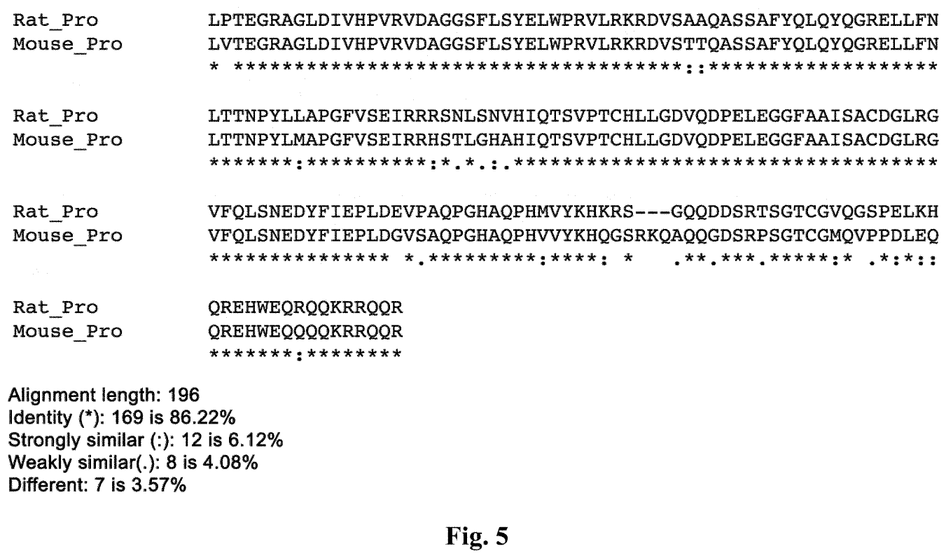

In some embodiments according to the first biological aspect the first portion of the polypeptide comprises a mutation within the furin cleavage site. FIG. 4 shows that furin cleavage site mutants of ADAMTS-7 improved the yield of processed or unprocessed ADAMTS-7. For example, in some of these embodiments the motif RQQR is mutated to RQKR (Q216K). In some embodiments, the motif RQQR within the first portion of the polypeptide is altered, preferably into RQKR. Thus, in some embodiments the first portion comprises a mutation, e.g. at position 216, such as the mutation Q216K. This mutation was found to improve cleavage by Furin between the prodomain and the catalytic domain of ADAMTS-7, thereby leading to improved yields of processed ADAMTS-7 compared to the wild type (WT). (cf. example B1, cf. FIG. 4). Q216K mutation changes rat ADAMTS7 motif RQQR↓S to RQKR↓S, i.e. to a more optimized consensus to increase Furin processing of the zymogen into the active catalytic form.

In some embodiments the first portion comprises triple mutant R58A/R61A/R217A (rPro-hCD-3RA). FIG. 4 panel B shows that these mutations abolished the processing to generate unprocessed protein only. For example, R58A/R60A/R217A prevents all furin cleavage resulting in a zymogen form.

According to some embodiments of the first biological aspect the recombinant nucleic acid sequence encodes at least for a rat pro-domain of ADAMTS-7 (SEQ ID No. 1, residues 1-217) and a catalytic domain of human ADAMTS-7, wherein optionally within the rat pro-domain of ADAMTS-7 the motif RQQR is mutated to RQKR (SEQ ID No. 2, cf. residues 1-217). In some of these embodiments said rat pro-domain of ADAMTS-7 comprises or consists of a sequence according to SEQ ID No. 1, residues 1-217 or SEQ ID No. 2, residues 1-217 and/or said catalytic domain of human ADAMTS-7 comprises or consists of a sequence according to SEQ ID No. 1, residues 218-518 or SEQ ID No. 2, residues 218-518.

In some embodiments the polypeptide or a fragment thereof encoded by the recombinant nucleic acid is suited to cleave a peptide comprising standard residues 1-15 of the amino acid sequence of SEQ ID NO: 4. For example, the polypeptide is suited to cleave a peptide comprising standard residues 1-15 of the amino acid sequence of SEQ ID NO: 4 and/or SEQ ID No. 11 with a a kcat/KM of at least 20% of a corresponding kcat/KM of human ADAMTS-7.

The produced polypeptide (e.g., having both the first portion and the second portion, or having only the second portion) can have a catalytic activity close to that of human ADAMTS-7 enzyme, e.g., in terms of kcat/KM, which can be even higher than the kcat/KM of the human enzyme, or can be within 50%, 45%, 40%, 35%, 30%, 25%, 20%, 15%, 10%, or 5% of the kcat/KM of the human enzyme, for example when a peptide having the sequence of of SEQ ID No. 11 is used as the substrate.

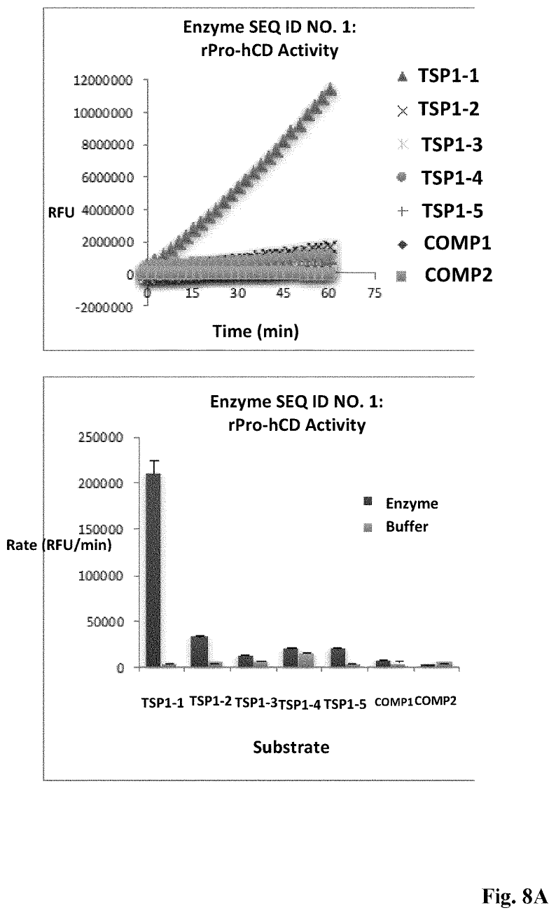

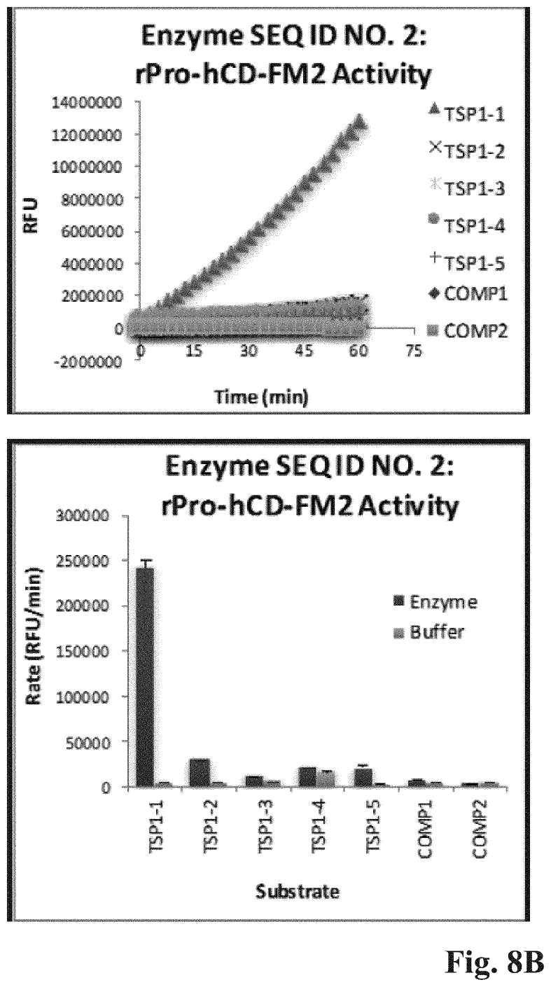

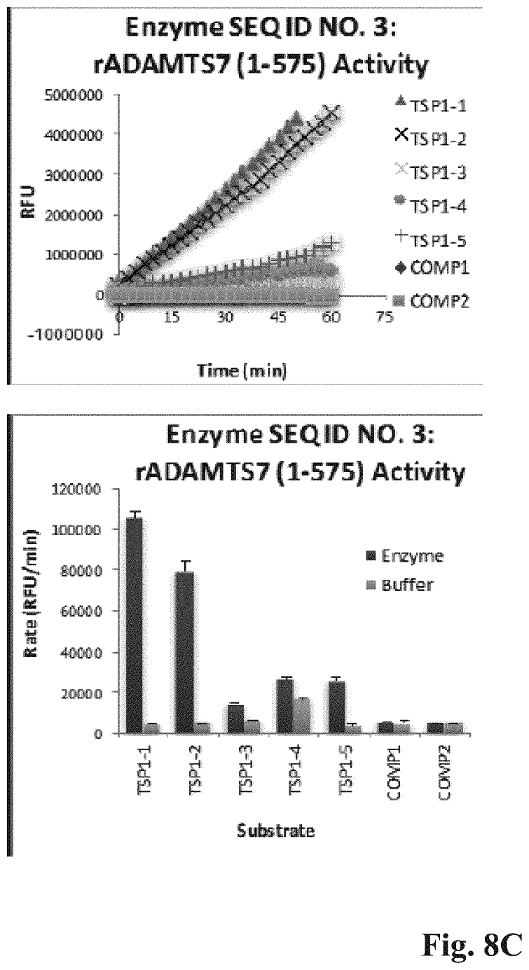

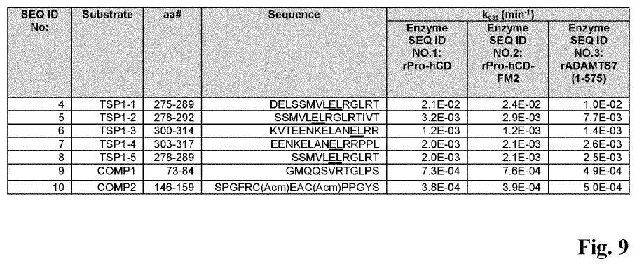

In some embodiments, fora given substrate, the produced polypeptide (e.g., having both the first portion and the second portion, or having only the second portion) has a catalytic activity in the same order of magnitude as rat ADAMTS-7, e.g., in terms of kcat, cf. FIGS. 8,9. In some embodiments, for a substrate of SEQ ID No. 4, the produced polypeptide has a kcat (min⁻¹) in the order of magnitude of 10⁻². In some embodiments, for a substrate of SEQ ID No. 5, the produced polypeptide has a kcat (min⁻¹) in the order of magnitude of 10⁻³.

Sequential cleavage or processing of ADAMTS-7 at furin cleavage sites by cellular enzyme furin leading to a complete removal of the Pro domain from the rest of the protein is likely a necessary step to a fully active or mature ADAMTS-7.

In some embodiments the recombinant nucleic acid sequence furthermore encodes for additional residues, such as a purification tag, such as a FLAG tag, a His tag, a Strep tag or any combination or repetition thereof. In some embodiments of the first biological aspect, the encoded polypeptide can have additional residues (e.g., purification tags such as His tag, Strep tag, 2×Strep tag, FLAG tag, 3×FLAG tag, and cleavage sequences such as TEV cleavage site). The presence of these additional residues is compatible with each of the described embodiments of the biological aspect. Purification of a polypeptide obtained from a nucleic acid according to the current invention can occur as described in example B3.

The recombinant nucleic acid according to the first biological aspect can be used for the expression of functional ADAMTS7 as described in example B3. For example, the construct can be inserted into a plasmid which is compatible with a certain expression system. In certain preferred embodiments, the construct can be cloned into a plasmid, preferably into a mammalian expression vector, such as pcDNA6mycHis. Expression can be performed in a compatible expression system, such as in a mammalian expression system, such as in HEK cells or Expi293 cells as known in the art. The Gibco Expi293 Expression System is a commercially available high-yield transient expression system based on suspension-adapted Human Embryonic Kidney (HEK) cells.

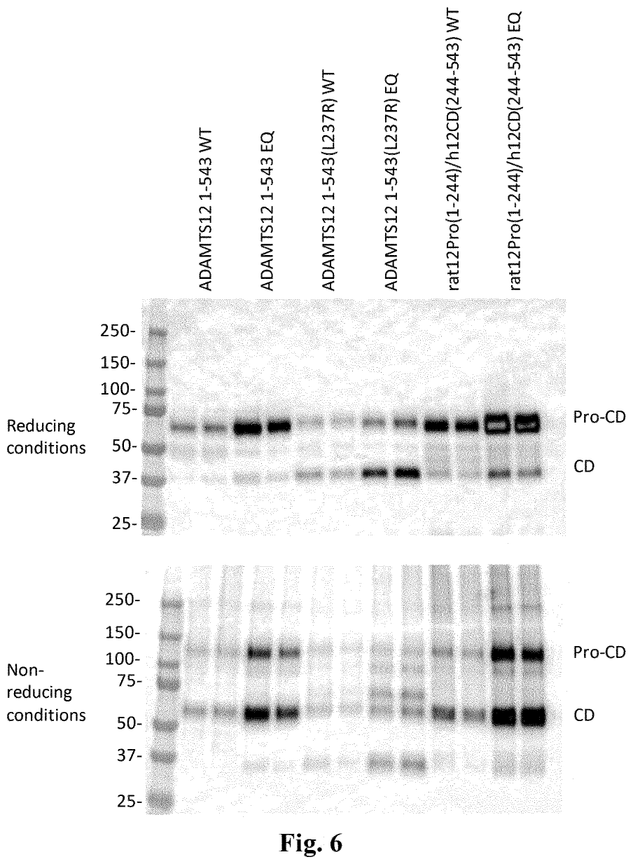

According to a second biological aspect of the current invention, there is provided a recombinant nucleic acid for the expression of functional ADAMTS-12. It was surprisingly found that human ADAMTS-12 expression is improved when a non human prodomain is used (cf. example B9, cf. FIG. 6). In particular it was found that human ADAMTS-12 with a rodent prodomain demonstrated a better expression profile compared to human ADAMTS-12 with a human prodomain. The recombinant nucleic acids according to the second biological aspect are therefore suited for the improved production of sufficient amounts of ADAMTS-12 with reproducible activity and purity. The resulting recombinant functional ADAMTS-12 can be used in an assay for the characterization of modulators of ADAMTS-7, i.e. where the selectivity for ADAMTS-7 is assessed.

According to some first embodiments according to the second biological aspect, there is provided a recombinant nucleic acid for expression of an ADAMTS-12 polypeptide that comprises a rodent prodomain of ADAMTS-12 as a first portion and a functional human ADAMTS-12 as a second portion.

In some embodiments according to the second biological aspect, the recombinant nucleic acid sequence encodes for a recombinant polypeptide that comprises a first portion and a second portion, wherein the first portion has a sequence identity of >80% with the sequence of residues 1-244 of SEQ ID NO: 15, and the second portion has a sequence identity of >80% with the sequence of residues 245-547 of SEQ ID NO: 15 (cf. 241-544 of ADAMTS12).

For example, ADAMTS12 rat/human hybrid can be made by fusing respective rat ADAMTS12 SP-Pro (1-244) followed by human ADAMTS12 CD (241-544).

In some embodiments according to the second biological aspect, the recombinant nucleic acid sequence encodes for a recombinant polypeptide that comprises a first portion and a second portion, wherein the first portion has a sequence identity of >90% with the sequence of residues 1-244 of SEQ ID NO: 15, and the second portion has a sequence identity of >90% with the sequence of residues 245-547 of SEQ ID NO: 15.

In some embodiments according to the second biological aspect, the recombinant nucleic acid sequence encodes for a recombinant polypeptide that comprises a first portion and a second portion, wherein the first portion has a sequence identity of >95% with the sequence of residues 1-244 of SEQ ID NO: 15, and the second portion has a sequence identity of >95% with the sequence of residues 245-547 of SEQ ID NO: 15.

In some embodiments according to the second biological aspect, the recombinant nucleic acid sequence encodes for a recombinant polypeptide that comprises a first portion and a second portion, wherein the first portion has a sequence identity of >98% with the sequence of residues 1-244 of SEQ ID NO: 15, and the second portion has a sequence identity of >98% with the sequence of residues 245-547 of SEQ ID NO: 15.

In some embodiments, according to the second biological aspect, the recombinant nucleic acid sequence encodes for a recombinant polypeptide that comprises a first portion and a second portion, wherein the first portion comprises residues 1-244 of SEQ ID NO: 15, and/or the second portion comprises residues 245-547 of SEQ ID NO: 15. In some different or the same embodiments the second portion is immediately C-terminal to the first portion.

In some embodiments according to the second biological aspect, the recombinant nucleic acid encodes a polypeptide that has a first portion and a second portion. The first portion of the polypeptide, in some embodiments, has an amino acid sequence that when aligned with an amino acid sequence of an ADAMTS-12 prodomain (or a fragment thereof) from a first species generates a Needleman-Wunsch score greater than 800 if BLOSUM62 matrix, a gap opening penalty of 11, and a gap extension penalty of 1 are used. In some embodiments, this generated Needleman-Wunsch score is greater than 850, 900, 950, 1000, 1050, 1100, 1150, 1200, 1250, or 1300. In some embodiments, the first portion and the ADAMTS-12 prodomain share a sequence identity that is at least 70%, 75%, 80%, 85%, 90%, 95%, 97% or 98%. The second portion of the polypeptide, in some embodiments, has an amino acid sequence that when aligned with an amino acid sequence of a human ADAMTS-12 catalytic domain (or a fragment thereof) generates a Needleman-Wunsch score greater than 1000 if BLOSUM62 matrix, a gap opening penalty of 11, and a gap extension penalty of 1 are used. In some embodiments, this generated Needleman-Wunsch score is greater than 1050, 1100, 1150, 1200, 1250, 1300, 1350, 1400, 1450, 1500, 1550, 1600, or 1650. In some embodiments, the second portion and the ADAMTS-12 catalytic domain share a sequence identity that is at least 70%, 75%, 80%, 85%, 90%, 95%, 97% or 98%.

In some embodiments according to the second biological aspect, the recombinant nucleic acid for expression of an ADAMTS-12 polypeptide encodes for a recombinant polypeptide that comprises a first portion having an amino acid sequence that aligns with an amino acid sequence of a ADAMTS-12 prodomain or a fragment thereof from anon-human species with a Needleman-Wunsch score greater than 800 when BLOSUM62 matrix, a gap opening penalty of 11, and a gap extension penalty of 1 are used; and a second portion having an amino acid sequence that aligns with an amino acid sequence of a ADAMTS-12 catalytic domain or a fragment thereof from a second species with a Needleman-Wunsch score greater than 1000 when BLOSUM62 matrix, a gap opening penalty of 11, and a gap extension penalty of 1 are used.

In some embodiments, the first species is a non-human species, such as a rodent species, such as rat. In some different or the same embodiments the second species is human, e.g. the catalytic domain is derived from human ADAMTS-12. In some embodiments the first species is a rodent species and the second species is human.

In some embodiments the second portion comprises a mutation at position E393, such as E393Q (EQ). This mutation results in increased protein yield similar to ADAMTS-7 catalytic mutations. Mutation numbering E393Q follows the species based positions for the human ADAMTS12 region of the construct, i.e. rat ADAMTS12 SP-Pro (1-244) followed by human ADAMTS12 CD (241-544).

In some embodiments the polypeptide or a fragment thereof encoded by the recombinant nucleic acid is suited to cleave a peptide comprising standard residues 1-15 of the amino acid sequence of SEQ ID NO: 4. For example, in some embodiments the polypeptide is suited to cleave a peptide comprising standard residues 1-15 of the amino acid sequence of SEQ ID NO: 4 and/or SEQ ID No. 11 with a a kcat/KM of at least 20% of a corresponding kcat/KM of human ADAMTS-12.

The produced polypeptide (e.g., having both the first portion and the second portion, or having only the second portion) can have a catalytic activity close to that of human ADAMTS-12 enzyme (e.g., in terms of kcat/KM, which can be even higher than the kcat/KM of the human enzyme, or can be within 50%, 45%, 40%, 35%, 30%, 25%, 20%, 15%, 10%, or 5% of the kcat/KM of the human enzyme, for example when a peptide having the sequence of of SEQ ID No. 4 or 11 is used as the substrate.

In some embodiments the recombinant nucleic acid sequence furthermore encodes for additional residues such as a purification tag, such as a FLAG tag, a His tag, a Strep tag or any combination or repetition thereof. In some embodiments of the second biological aspect, the encoded polypeptide can have additional residues (e.g., purification tags such as His tag, Strep tag, 2×Strep tag, FLAG tag, 3×FLAG tag, and cleavage sequences such as TEV cleavage site). The presence of these additional residues is compatible with each of the described embodiments of the biological aspect. Purification of the functional ADAMTS-12 obtained from a recombinant nucleic acid according to the current invention can occur as described in example B4. According to some embodiments of the second biological aspect the recombinant nucleic acid sequence encodes at least for a rat pro-domain of ADAMTS-12, such as amino acids 1-244 of rat sequence UniProt D3ZTJ3 and/or encodes fora catalytic domain of human ADAMTS-12, such as amino acids 241-543 of human sequence UniProt P58397, cf. example B1. In certain preferred embodiments, said recombinant nucleic acid encodes for a sequence according to SEQ ID No. 15.

The recombinant nucleic acid according to the second biological aspect can be used for the expression of functional ADAMTS-12 as described in example B4. For example, the recombinant nucleic acid can be inserted into a plasmid which is compatible with a certain expression system. In certain preferred embodiments, the construct can be inserted into a plasmid, preferably into a mammalian expression vector, such as pcDNA3.4. Expression can be performed in a compatible expression system, such as in a mammalian expression system, such as in HEK cells or Expi293 cells as known in the art. The Gibco Expi293 Expression System is a commercially available high-yield transient expression system based on suspension-adapted Human Embryonic Kidney (HEK) cells.

According to a third biological aspect of the current invention, there is provided a recombinant polypeptide, wherein the recombinant polypeptide is the recombinant polypeptide encoded by a recombinant nucleic acid according to the first or second biological aspect, or a fragment thereof. In some embodiments the fragment is the processed polypeptide which results after Furin cleavage. In some embodiments, the Furin cleavage occurs at the site known in the art or described herein.

In some embodiments the recombinant polypeptide according to the third biological aspect or a fragment thereof is suited to cleave a peptide substrate comprising standard residues 1-15 of SEQ ID NO: 4. The recombinant polypeptide according to the third biological aspect or a fragment thereof can be used in an assay for the identification and characterization of modulators of ADAMTS-7 and/or ADAMTS-12, as shown within the examples.

In some embodiments, the recombinant polypeptide according to the third biological aspect or a fragment thereof is a functional ADAMTS-7 protein. In some embodiments the polypeptide comprises a rodent prodomain of ADAMTS-7 as a first portion and a functional human ADAMTS-7 as a second portion.

In some embodiments the polypeptide comprises a first portion and a second portion, wherein the first portion has a sequence identity of >80% with the sequence of residues 1-217 of SEQ ID NO: 1 or with the sequence of residues 1-217 of SEQ ID NO: 2, and the second portion has a sequence identity of >80% with the sequence of residues 218-518 of SEQ ID NO: 1.

In some embodiments according to the third biological aspect, the recombinant polypeptide comprises a first portion having an amino acid sequence that aligns with an amino acid sequence of an ADAMTS-7 prodomain or a fragment thereof from a first species with a Needleman-Wunsch score greater than 700, when BLOSUM62 matrix, a gap opening penalty of 11, and a gap extension penalty of 1 are used; and a second portion having an amino acid sequence that aligns with an amino acid sequence of a ADAMTS-7 catalytic domain or a fragment thereof from a second species with a Needleman-Wunsch score greater than 1000 when BLOSUM62 matrix, a gap opening penalty of 11, and a gap extension penalty of 1 are used. In some embodiments the first species is a rodent species such as rat and the second species is human.

In some embodiments the first portion of the polypeptide comprises a mutation within the furin cleavage site. In some embodiments, the motif RQQR within the first portion of the polypeptide is altered, preferably into RQKR. Thus, in some embodiments the first portion comprises a mutation, e.g. at position 216, such as the mutation Q216K. This mutation was found to improve cleavage by Furin between the prodomain and the catalytic domain of ADAMTS-7, thereby leading to improved yields of processed ADAMTS-7 compared to the wild type (WT). (cf. example B1, cf. FIG. 4).

In some embodiments the polypeptide comprises a rodent prodomain of ADAMTS-12 as a first portion and a functional human ADAMTS-12 as a second portion.

In some embodiments the polypeptide comprises a first portion and a second portion, wherein the first portion has a sequence identity of >80% with the sequence of residues 1-244 of SEQ ID NO: 15, and the second portion has a sequence identity of >80% with the sequence of residues 245-547 of SEQ ID NO: 15.

In some embodiments according to the current biological aspect, the polypeptide comprises a first portion having an amino acid sequence that aligns with an amino acid sequence of a ADAMTS-12 prodomain or a fragment thereof from a first species with a Needleman-Wunsch score greater than 800 when BLOSUM62 matrix, a gap opening penalty of 11, and a gap extension penalty of 1 are used; and a second portion having an amino acid sequence that aligns with an amino acid sequence of a ADAMTS-12 catalytic domain or a fragment thereof from a second species with a Needleman-Wunsch score greater than 1000 when BLOSUM62 matrix, a gap opening penalty of 11, and a gap extension penalty of 1 are used. In some embodiments, the first species is a non-human species, such as a rodent species, such as rat. In some different or the same embodiments the second species is human, e.g. the catalytic domain is derived from human ADAMTS-12. In some embodiments the first species is a rodent species and the second species is human.

In some embodiments the second portion comprises a mutation at position E393, such as E393Q (EQ). This mutation results in increased protein yield.

In some embodiments the polypeptide (e.g., having both the first portion and the second portion, or having only the second portion) has a catalytic activity close to that of human ADAMTS-7 or ADAMTS-12 enzyme (e.g., in terms of kcat/KM, which can be even higher than the kcat/KM of the human enzyme, or can be within 50%, 45%, 40%, 35%, 30%, 25%, 20%, 15%, 10%, or 5% of the kcat/KM of the human enzyme, for example when a peptide having the sequence of of SEQ ID No. 4 or 11 is used as the substrate.

In some embodiments the polypeptide or a fragment thereof is suited to cleave a peptide comprising standard residues 1-15 of the amino acid sequence of SEQ ID NO: 4. In some embodiments, the polypeptide is suited to cleave a peptide comprising standard residues 1-15 of the amino acid sequence of SEQ ID NO: 4 and/or SEQ ID No. 11 with a a kcat/KM of at least 20% of a corresponding kcat/KM of human ADAMTS-7 or human ADAMTS-12.

In some embodiments the polypeptide comprises a purification tag, such as a FLAG tag, a His tag, a Strep tag or any combination or repetition thereof. In some embodiments of the first biological aspect, the encoded polypeptide can have additional residues (e.g., purification tags such as His tag, Strep tag, 2×Strep tag, FLAG tag, 3×FLAG tag, and cleavage sequences such as TEV cleavage site). The presence of these additional residues is compatible with each of the described embodiments of the biological aspect.

Surprisingly, the recombinant polypeptide according to the current biological aspect was found to fold into a functional ADAMTS, e.g. ADAMTS-7 or ADAMTS-12. Expression and purification of recombinant functional ADAMTS protein according to the third biological aspect can occur as described in example B3.

According to a fourth biological aspect of the current invention there is provided a recombinant peptide substrate for ADAMTS-7 and/or ADAMTS-12. The peptide substrate according to the current invention can be used as an artificial substrate for ADAMTS-7 and/or ADAMTS-12. The peptide substrate can furthermore be used in order to determine the proteolytic activity of ADAMTS-7 and/or ADAMTS-12, identify modulators of ADAMTS-7 and/or ADAMTS-12, and/or to determine the degree of modulation induced by an agonist or antagonist.

In some embodiments the peptide substrate comprises a subsequence of a natural ADAMTS-7 and/or ADAMTS-12 substrate, such as a subsequence of TSP1 or COMP. In some embodiments, the peptide substrate according to the fourth biological aspect comprises at least a sequence according to any of SEQ ID No. 4, 5, 8, 11, 12 or 13, or a fragment thereof comprising the amino acids EL. It was surprisingly found that theses sequences can be used as cleavage site for ADAMTS-7 and ADAMTS-12: For example, the sequence DELSSMVLELRGLRT, derived from TSP1, residues 275-289 has been surprisingly identified as a suitable substrate for ADAMTS-7 and ADAMTS-12. Without being bound by theory, cleavage occurs between Glu283 and Leu284 (EL).

In some embodiments the peptide substrate comprises (a) residues 1-15 of the amino acid sequence of SEQ ID NO: 4, or (b) residues 1-15 of the amino acid sequence of SEQ ID NO: 5, or (c) residues 1-13 of the amino acid sequence of SEQ ID NO: 8, or (d) a fragment of any of the sequences according to a), b) or c), the fragment comprising the amino acid sequence EL. In some embodiments, the peptide substrate according to the fourth biological aspect comprises at least the amino acids EL. In some embodiments, the peptide substrate according to the fourth biological aspect comprise at least the SEQ ID No. 4, or a fragment thereof comprising the amino acids EL. In some embodiments, the peptide substrate according to the fourth biological aspect comprises at least the SEQ ID No. 5, or a fragment thereof comprising the amino acids EL. In some embodiments, the peptide substrate according to the fourth biological aspect comprises at least the SEQ ID No. 8, or a fragment thereof comprising the amino acids EL.

In some embodiments, the peptide substrate according to the fourth biological aspect comprises at least the SEQ ID No. 11, 12 or 13 or a fragment thereof comprising the amino acids EL. It was surprisingly found that the solubility of the described peptide substrate according to the fourth biological aspect could be improved by including an additional hydrophilic moiety without affecting the activity profile (cf. SEQ ID No. 11, 12, 13).

In some embodiments the peptide substrate according to the fourth biological aspect comprises a first moiety conjugated to a residue that is N-terminal to sequence fragment EL as comprised within SEQ ID No. 4, 5, or 8 or the fragment thereof, and a second moiety conjugated to a residue that is C-terminal to said sequence fragment EL. In some embodiments the first moiety comprises a fluorophore and the second moiety comprises a quencher, or the first moiety comprises a quencher and the second moiety comprises a fluorophore.

Without being bound by theory, the fluorophore of the peptide substrate can be excited at a suitable wavelength, e.g. by exposing it to light. The suitability of a wavelength depends on the specific fluorophore and can be determined as known in the art. Without being bound by theory, the excited fluorophore transfers the energy to the closely located quencher, which has the ability to decrease the fluorescence intensity of the fluorophore, such that no fluorescence or only background fluorescence is detected at the emission wavelength of the fluorophore. If the peptide substrate according to the current invention is however exposed to or contacted with a functional ADAMTS-7 or ADAMTS-12, the enzyme cleaves the peptide, thereby separating fluorophore and quencher. In the absence of the quencher, the excited fluorophore emits light in returning to the ground state and an increase in fluorescence can be detected.

Using a combination of functional ADAMTS-7 and the peptide substrate comprising a fluorophore and a quencher according to the fourth biological aspect as a substrate thus allows for the robust and reproducible identification and characterization of ADAMTS-7 modulators and/or ADAMTS-12 modulators.

The skilled person understands that according to the various biological aspects and embodiments of the current invention, multiple sites can be used to attach the fluorophore aid the quencher to the peptide as long as a) the distance between fluorophore and quencher allows for the transfer of energy between fluorophore and quencher and b) the ADAMTS-7 cleavage site is arranged in such a way that fluorophore and quencher are separated upon ADAMTS-7 cleavage of the peptide. The latter effect can be obtained by interposing the ADAMTS-7 cleavage site between the fluorophore and the quencher.

A variety of suitable pairs of fluorophores and quenchers have been described in the literature. The skilled person is well aware which pairs of fluorophore and quencher can be combined. To obtain a peptide according to the biological aspect at hand, the distance between fluorophore and quencher has to be adjusted to allow for the necessary transfer of energy as described herein. The specific distance depends on the specific selection of fluorophore and quencher ad can be adjusted as known in the art. For example in some embodiments according to the biological aspect at hand, EDANS as a fluorophore can be paired with DABCYL or DABSYL as a quencher. When EDANS and DABCYL are in a close proximity (10-100 Å), the energy emitted from EDANS will be quenched by Dabcyl, resulting in low or no fluorescence. However, if the compounds are separated EDANS will fluoresce. The optimal absorbance and emission wavelengths of EDANS are Aabs=336 nm and Aem=490 nm respectively, and for Dabcyl, the maximum absorbance wavelength is Aabs=472 nm, which, to a large extent, overlap with the emission spectra of EDANS.

Another pair of fluorophore and quencher where the compounds can be used alone or in combination is 7-methoxy-coumarin-4-yl acetic acid (MCA) as the fluorophore with Lys(DNP) as a quencher. In some preferred embodiments according to the biological aspect at hand the fluorophore is MCA and the quencher is Lys(DNP). A further pair which can be used according to the current invention comprises 7-amino-4-carbamoylmethylcoumarin (ACC) as the fluorophore and 2,4-dinitrophenyl-lysine (Lys(DNP)) as the quencher. In some preferred embodiments according to the biological aspect at hand the fluorophore is ACC and the quencher is Lys(DNP). Another pair of fluorophore and quencher that can be used alone or in combination is HiLyteFluor, e.g. HiLyteFluor-488 as the fluorophore with QXL, e.g. QXL520 as a quencher. HiLyte Fluor fluorophores are commercially available for various wavelengths and can be prepared as known in the art (Jungbauer, L M et al. “Preparation of fluorescently-labeled amyloid-beta peptide assemblies: the effect of fluorophore conjugation on structure and function” Journal of molecular recognition: JMR vol. 22.5 (2009): 403-13.). QXL quenchers are commercially available for various wavelengths and can be prepared as known in the art. QXL 570 dyes are optimized quenchers for rhodamines (such as TAM RA, sulforhodamine B, ROX) and Cy3 fluorophores. Their absorption spectra overlap with the fluorescence spectra of TAM RA, sulforhodamine B, ROX and Cy3.

According to some embodiments according to the fourth biological aspect said fluorophore is HiLyteFluor, such as HiLyteFluor-488 and the quencher is a matching QXL quencher, such as QXL520.

According to some embodiments according to the fourth biological aspect said peptide substrate comprises or consists of a subsequence of a natural ADAMTS-7 and/or ADAMTS-12 substrate, such as a subsequence of TSP1 or COMP, such as (a) residues 1-15 of the amino acid sequence of SEQ ID NO: 4, or (b) residues 1-15 of the amino acid sequence of SEQ ID NO: 5, or (c) residues 1-13 of the amino acid sequence of SEQ ID NO: 8, or (d) a fragment of any of the sequences according to a), b) or c), the fragment comprising the amino acid sequence EL, said fluorophore is HiLyteFluor, such as HiLyteFluor-488 and the quencher is a matching QXL quencher such as QXL520.

According to some embodiments according to the fourth biological aspect said peptide substrate comprises or consists of a subsequence of a natural ADAMTS-7 and/or ADAMTS-12 substrate, such as a subsequence of TSP1 or COMP, such as (a) residues 1-15 of the amino acid sequence of SEQ ID NO: 4, or (b) residues 1-15 of the amino acid sequence of SEQ ID NO: 5, or (c) residues 1-13 of the amino acid sequence of SEQ ID NO: 8, or (d) a fragment of any of the sequences according to a), b) or c), the fragment comprising the amino acid sequence EL, said fluorophore is MCA and said quencher is Lys(DNP).

According to some embodiments according to the fourth biological aspect said peptide substrate comprises or consists of a subsequence of a natural ADAMTS-7 and/or ADAMTS-12 substrate, such as a subsequence of TSP1 or COMP, such as (a) residues 1-15 of the amino acid sequence of SEQ ID NO: 4, or (b) residues 1-15 of the amino acid sequence of SEQ ID NO: 5, or (c) residues 1-13 of the amino acid sequence of SEQ ID NO: 8, or (d) a fragment of any of the sequences according to a), b) or c), the fragment comprising the amino acid sequence EL, said fluorophore is ACC and said quencher is Lys(DNP).

In some embodiments, the fluorophore, such as HiLyte fluor 488 is attached to the N-terminus of the peptide and the quencher, such as QXL520, is attached to the C-terminus or vice versa. In certain preferred embodiments, according to the biological aspect at hand, an additional negative residue, such as carboxyl position glutamic acid is attached C-terminal of the quencher, e.g. after the QXL520 quencher. The addition of the residue improved the solubility behavior of the peptide substrate and thereby lead to an improved reproducibility of the assay.

According to some embodiments according to the biological aspect at hand said peptide substrate for ADAMTS-7 and/or ADAMTS-12 comprises or consists of a subsequence of a natural ADAMTS-7 substrate, such as a subsequence of TSP1 or COMP, such as (a) residues 1-15 of the amino acid sequence of SEQ ID NO: 4, or (b) residues 1-15 of the amino acid sequence of SEQ ID NO: 5, or (c) residues 1-13 of the amino acid sequence of SEQ ID NO: 8, or (d) a fragment of any of the sequences according to a), b) or c), the fragment comprising the amino acid sequence EL, and the peptide substrate is further characterized in comprising an additional negatively charged residue such as a carboxyl position glutamic acid, e.g. C-terminal of the quencher.

According to some embodiments according to the biological aspect at hand said peptide substrate for ADAMTS-7 and/or ADAMTS-12 comprises or consists of a subsequence of a natural ADAMTS-7 substrate, such as a subsequence of TSP1 or COMP, such as (a) residues 1-15 of the amino acid sequence of SEQ ID NO: 4, or (b) residues 1-15 of the amino acid sequence of SEQ ID NO: 5, or (c) residues 1-13 of the amino acid sequence of SEQ ID NO: 8, or (d) a fragment of any of the sequences according to a), b) or c), the fragment comprising the amino acid sequence EL, said fluorophore is HiLyteFluor, such as HiLyteFluor-488, said quencher is a matching QXL quencher such as QXL520 and the peptide substrate is further characterized in comprising an additional negatively charged residue such as a carboxyl position glutamic acid, e.g. C-terminal of the quencher.

According to a fifth biological aspect of the current invention there is provided a method for the identification or characterization of an ADAMTS-7 and/or ADAMTS-12 modulator comprising the steps of
a) contacting a recombinant polypeptide or a fragment thereof according to the third biological aspect, and
b) contacting said recombinant polypeptide or fragment thereof with a peptide substrate according to the fourth biological aspect, wherein the peptide substrate comprises a fluorophore and a quencher, and
c) detecting fluorescence as a measure for the activity of said recombinant polypeptide or a fragment thereof.

In some embodiments, the recombinant polypeptide is suited to cleave a peptide comprising standard residues 1-15 of the amino acid sequence of SEQ ID NO: 4.

In some embodiments, the recombinant polypeptide comprises a first portion and a second portion, the first portion having a sequence identity of at least 80% with the sequence of residues 1-217 of SEQ ID NO: 1 and/or having a sequence identity of at least 80% with the sequence of residues 1-217 of SEQ ID NO: 2, and the second portion having a sequence identity of >80% with the sequence of residues 218-518 of SEQ ID NO: 1 and the peptide substrate comprises (a) residues 1-15 of the amino acid sequence of SEQ ID NO: 4, or (b) residues 1-15 of the amino acid sequence of SEQ ID NO: 5, or (c) residues 1-13 of the amino acid sequence of SEQ ID NO: 8, or (d) a fragment of any of the sequences according to a), b) or c), the fragment comprising the amino acid sequence EL.

In some embodiments, the recombinant polypeptide comprises a first portion and a second portion, the first portion having a sequence identity of >80% with the sequence of residues 1-244 of SEQ ID NO: 15, and the second portion having a sequence identity of >80% with the sequence of residues 245-547 of SEQ ID NO: 15 and the peptide substrate comprises (a) residues 1-15 of the amino acid sequence of SEQ ID NO: 4, or (b) residues 1-15 of the amino acid sequence of SEQ ID NO: 5, or (c) residues 1-13 of the amino acid sequence of SEQ ID NO: 8, or (d) a fragment of any of the sequences according to a), b) or c), the fragment comprising the amino acid sequence EL.

In some embodiments, the recombinant polypeptide comprises a sequence according to SEQ ID No. 01, SEQ ID No. 02 or SEQ ID No. 15 and/or the peptide substrate comprises (a) residues 1-15 of the amino acid sequence of SEQ ID NO: 4, or (b) residues 1-15 of the amino acid sequence of SEQ ID NO: 5, or (c) residues 1-13 of the amino acid sequence of SEQ ID NO: 8, or (d) a fragment of any of the sequences according to a), b) or c), the fragment comprising the amino acid sequence EL, and optionally comprises a further carboxy group.

Contacting of different compounds can be performed by preparing a solution comprising the respective compounds, e.g. in an appropriate concentration, e.g. as described in the examples. The test compound can be any molecule, such as any small molecule provided for throughout this application.

There is no specific order with regard to the steps of the method, except for the obvious restrictions resulting from the mode of action. In some embodiments the method according to the biological aspect at hand is further characterized in that step c) is performed for a solution comprising said recombinant polypeptide, said test compound, and said peptide substrate.

In some embodiments, the method according to the biological aspect at hand can be used to determine the half maximal inhibitory concentration (IC50) or the half maximal effective concentration (EC50) of an ADAMTS-7 and/or ADAMTS-12 modulator. According to the FDA, IC50 represents the concentration of a drug that is required for 50% inhibition in vitro. The determined IC50 as described herein is a measure of the potency of the test compound in inhibiting ADAMTS-7 and/or ADAMTS-12 induced cleavage of a natural or artificial substrate. Half maximal effective concentration (EC50) refers to the concentration of a test compound which induces a response halfway between the baseline and maximum after a specified exposure time and can be used as a measure of agonistic potency of a compound. IC50 and EC50 can be determined as known in the art. In brief, the IC50 of a drug can be determined by constructing a dose-response curve and analyzing the resulting fluorescence for different concentrations of the test compound.

In some embodiments according to the fifth biological aspect a test compound can be or is selected as a modulator (e.g. agonist or inhibitor) if after contacting the recombinant polypeptide with the at least one test compound according to step a) and after contacting said recombinant polypeptide with a peptide substrate according to step b) no significant increase of fluorescence is detected or if an increase of fluorescence is detected which is significantly lower than the increase of fluorescence which is detectable for a control in the absence of the test compound. In some embodiments according to the fifth biological aspect a test compound is selected as an ADAMTS-7 and/or ADAMTS-12 agonist if after contacting the recombinant polypeptide with the at least one test compound according to step a) and after contacting said recombinant polypeptide with a peptide substrate according to step b) an increase of fluorescence is detected which is significantly higher than the increase of fluorescence which is detectable for a control in the absence of the test compound. An exemplary way how the method can be performed is described in example B5.

According to some embodiments according to the current biological aspect there is provided a method for evaluating the selectivity of an ADAMTS-7 and/or ADAMTS-12 modulator comprising the method according to the fifth biological aspect further characterized in comprising the steps of
a) contacting a functional recombinant metalloproteinase different from ADAMTS-7 and ADAMTS-12 with the at least one test compound, and
b) contacting said functional recombinant metalloproteinase with a peptide substrate comprising a fluorophore and a quencher, wherein said peptide substrate comprises a cleavage site for said functional recombinant metalloproteinase different from ADAMTS-7 and/or ADAMTS-12; and
c) detecting the fluorescence as a measure for the activity of functional recombinant metalloproteinase different from ADAMTS-7 and ADAMTS-12.

In certain preferred embodiments, the functional recombinant metalloproteinase is different from ADAMTS-7 and ADAMTS-12 is ADAMTS4, ADAMTS5, MMP12, MMP15, MMP2 or ADAM17 or any other enzyme listed in table 2. In some preferred embodiments, the peptide comprising a fluorophore and a quencher is the peptide as specified in table 2 and/or comprises a cleavage site of said functional recombinant metalloproteinase different from ADAMTS-7 and/or ADAMTS-12 as disclosed in table 2.

For characterization of the inhibition, based on the fluorescence IC50 values can be calculated from percentage of inhibition of enzyme activity as a function of test compound concentration. According to a sixth biological aspect there is provided a modulator of ADAMTS-7 and/or ADAMTS-12 identified by a method according to the fifth biological aspect for use in the treatment of heart disease, vascular disease, and/or cardiovascular disease, including atherosclerosis, coronary artery disease (CAD), myocardial infarction (MI), peripheral vascular disease (PAD)/arterial occlusive disease and/or restenosis after angioplasty (including the use of drug-coated or non drug-coated balloons and/or stent-implantation) and/or for the treatment and/or prophylaxis of lung disease, inflammatory disease, fibrotic disease, metabolic disease, cardiometabolic disease and/or diseases/disease states affecting the kidneys and/or the central nervous and/or neurological system as well as gastrointestinal and/or urologic and/or ophthalmologic disease/disease states.

For these diseases or disease states, alterations or aberrant expression with regard to ADAMTS-7 and/or ADAMTS-12 have been described earlier. In some embodiments the modulator of ADAMTS-7 and/or ADAMTS-12 is a modulator for use in the treatment of coronary artery disease (CAD), peripheral vascular disease (PAD) and myocardial infarction (MI). In some embodiments, the modulator according to the sixth biological aspect is a small molecule. In some embodiments, the modulator according to the seventh biological aspect is an antagonist of human ADAMTS-7. In some different or the same embodiments, the modulator according to the sixth biological aspect is an antagonist of human ADAMTS-12. In some different or the same embodiments, the modulator according to the sixth biological aspect is an antagonist of human ADAMTS4. In some embodiments the modulator according to the sixth biological aspect is a small molecule, e.g. as provided in the examples. While the identification of selective ADAMTS-7 and ADAMTS-12 modulators has been extremely challenging in the past, the provided assay now offers all means to obtain these moulators according to the current biological aspect. According to a seventh biological aspect there is provided a method of producing a recombinant polypeptide according to any of the previous biological aspects, the method comprising cultivating a recombinant host cell comprising a recombinant nucleic acid according to any biological aspect described herein and recovering the recombinant polypeptide of a fragment thereof.

According to an eighth biological aspect there is provided a kit of parts comprising a recombinant nucleic acid according to the first and/or second biological aspect and/or a polypeptide according to the third biological aspect and a peptide substrate according to the fourth biological aspect. In some embodiments the kit of parts can be used to perform a method according to the fifth biological aspect in a convenient and reproducible way. In some embodiments the provided kit of parts can be used to evaluate whether a test molecule is a modulator of ADAMTS-7. In some different or the same embodiments the provided kit of parts can be used to evaluate whether a test molecule is a modulator of ADAMTS-12. In some embodiments the provided kit of parts can be used to evaluate whether a test molecule is a modulator of ADAMTS-7 and a modulator of ADAMTS-12.

### EXPERIMENTAL SECTION

The following table 1 lists the abbreviations used in this paragraph and in the Examples section as far as they are not explained within the text body. Other abbreviations have their meanings customary per se to the skilled person.

TABLE 1
Abbreviations
The following table lists the abbreviations used herein.

| Abbreviation | Meaning |
|---|---|
| Ac | acetate |
| acac | acetylacetonate |
| ACN | acetonitrile |
| aq. | aqueous |
| bp | broad peak |
| br | broad (¹H-NMR signal) |
| Bu | butyl |
| CDI | carbonyldiimidazole |
| CI | chemical ionisation |
| conc. | concentrated |
| d | doublet (¹H-NMR signal) |
| DBU | 1,8-Diazabicyclo[5.4.0]undec-7-ene |
| DCM | dichloromethane |
| dd | double-doublet (¹H-NMR signal) |
| ddd | doublet of doublets of doublets |
| diamix | mixture of four stereoisomers containing diastereoisomers |
| | and enantiomers |
| DIPEA | diisopropylethylamine |
| DMA | dimethylacetamide |
| DMF | N,N-Dimethylformamide |
| DMSO | dimethylsulfoxide |
| EDC | 1-ethyl-3-(3-dimethylaminopropyl)carbodiimide |
| | hydrochloride |
| ent | enantiomerically pure compound |
| eq. | equivalents |
| ESI | electrospray (ES) ionisation |
| Et | ethyl |
| EtOH | ethanol |
| h | hour(s) |
| HATU | (1-[bis(dimethylamino)methylene)-1H-1,2,3-triazolo |
| | [4,5-b]pyridinium-3-oxide hexafluorophosphate |
| HOBt | 1-hydroxybenzotriazole hydrate |
| HPLC | high performance liquid chromatography |
| iPr | isopropyl |
| LC-MS | liquid chromatography mass spectrometry |
| LiHMDS | Lithium bis(trimethylsilyl)amide |
| m | multiplet (¹H-NMR signal) |
| MeOH | methanol |
| min | minute(s) |
| MS | mass spectrometry |
| N | normal (concentration) |
| NBS | N-bromosuccinimide |
| NCS | N-chlorosuccinimide |
| NMR | nuclear magnetic resonance spectroscopy: chemical |
| | shifts (5) are given in ppm. The chemical shifts were |
| | corrected by setting the DMSO signal to 2.50 ppm |
| | unless otherwise stated. |
| PCC | Pyridinium chlorochromate |
| q | quartet |
| rac | racemic mixture |
| RF | reflux |
| RP | Reversed-Phase chromatography |
| Rt | retention time (as measured either with HPLC or |
| | UPLC) in minutes |
| RT | Room Temperature |
| s | singlet (¹H-NMR signal) |
| sat. | saturated |
| SQD | Single-Quadrupole-Detector |
| t | triplet (¹H-NMR signal) |
| TBAF | tetrabutylammoniumfluoride |
| TBDMSCI | tert.-butyldimethylsilylchlorid |
| td | triple-doublet (¹H-NMR signal) |
| TEA | triethylamine |
| TFA | trifluoroacetic acid |
| THF | tetrahydrofuran |
| TLC | Thin Layer Chromatography |
| UPLC | ultra performance liquid chromatography |

### Experimental Section—General Part

All reagents, for which the synthesis is not described in the experimental part, are either commercially available, or are known compounds or may be formed from known compounds by known methods by a person skilled in the art.

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. Biotage SNAP cartidges KP-Sil® or KP-NH® in combination with a Biotage autopurifier system (SP4® or Isolera Four®) and eluents such as gradients of hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

Chemical names were generated using the ACD/Name software from ACD/Labs. In some cases generally accepted names of commercially available reagents were used in place of ACD/Name generated names.

The various aspects of the invention described in this application are illustrated by the following examples which are not meant to limit the invention in any way.

The example testing experiments described herein serve to illustrate the present invention aid the invention is not limited to the examples given.

### NMR Analytical Method:

NMR peak forms are stated as they appear in the spectra, possible higher order effects have not been considered.

The ¹H NMR data of selected synthesis intermediates and working examples are stated in the form of ¹H NMR peak lists. For each signal peak, first the δ[ppm]=value in ppm and then the signal intensity in round brackets are listed. The δ[ppm]=value/signal intensity number pairs for different signal peaks are listed with separation from one another by commas. The peak list for an example therefore takes the following form: δ[ppm]=₁ (intensity₁), δ[ppm]=₂ (intensity₂), . . . , δ[ppm]=ᵢ (intensityᵢ), . . . , δ[ppm]=ₙ (intensityₙ).

The intensity of sharp signals correlates with the height of the signals in a printed example of an NMR spectrum in cm and shows the true ratios of the signal intensities in comparison with other signals. In the case of broad signals, several peaks or the middle of the signal and the relative intensity thereof may be shown in comparison to the most intense signal in the spectrum. The lists of the ¹H NMR peaks are similar to the conventional ¹H NMR printouts and thus usually contain all peaks listed in a conventional NMR interpretation. In addition, like conventional ¹H NMR printouts, they may show solvent signals, signals of stereoisomers of the target compounds which are likewise provided by the invention, and/or peaks of impurities. The peaks of stereoisomers of the target compounds and/or peaks of impurities usually have a lower intensity on average than the peaks of the target compounds (for example with a purity of >90%). Such stereoisomers and/or impurities may be typical of the particular preparation process. Their peaks can thus help in identifying reproduction of our preparation process with reference to “by-product fingerprints”. An expert calculating the peaks of the target compounds by known methods (MestreC, ACD simulation, or using empirically evaluated expected values) can, if required, isolate the peaks of the target compounds, optionally using additional intensity filters. This isolation would be similar to the peak picking in question in conventional ¹H NMR interpretation. A detailed description of the presentation of NMR data in the form of peak lists can be found in the publication “Citation of NMR Peaklist Data within Patent Applications” (cf. Research Disclosure Database Number 605005, 2014, 1 Aug. 2014 or http://www.researchdisclosure.com/searching-disclosures). In the peak picking routine described in Research Disclosure Database Number 605005, the parameter “MinimumHeight” can be set between 1% and 4%. Depending on the type of chemical structure and/o r depending on the concentration of the compound to be analysed, it may be advisable to set the parameters “MinimumHeight” to values of <1%.

### LC-MS Procedures

### GENERAL REMARKS

Method 1-6 were performed using an AgilentG1956ALC/MSD quadrupole coupled to an Agilent 1100 series liquid chromatography (LC) system consisting of a binary pump with degasser, autosampler, thermostated column compartment and diode array detector. The mass spectrometer (MS) was operated with an atmospheric pressure electro-spray ionization (API-ES) source in positive ion mode (Mass method 1). The capillary voltage was set to 3000 V, the fragmentor voltage to 70 V and the quadrupole temperature was maintained at 100° C. The drying gas flow and temperature values were 12.0 L/min and 350° C., respectively. Nitrogen was used as the nebuliser gas, at a pressure of 35 psi. Data acquisition was performed with Agilent Chemstation software.

### Method 1:

Analyses were carried out on a YMC pack ODS-AQ C18 column (50 mm long×4.6 mm I.D.; 3 μm particle size) at 35° C., with a flow rate of 2.6 mL/min. A gradient elution was performed from 95% (Water+0.1% Formic acid)/5% Acetonitrile to 5% (Water+0.1% Formic acid)/95% Acetonitrile in 4.8 min; the resulting composition was held for 1.0 min; from 5% (Water+0.1% formic acid)/95% Acetonitrile to 95% (Water+0.1% formic acid)/5% Acetonitrile in 0.2 min. The standard injection volume was 2 μL. Acquisition ranges were set to 190-400 nm for the UV-PDA detector and 100-1400 m/z for the MS detector.

### Method 2:

Analyses were carried out on a Phenomenex Kinetex 00B-4475-AN C18 column (50 mm long×2.1 mm I.D.; 1.7 μm particles) at 60° C., with a flow rate of 1.5 mL/min. A gradient elution was performed from 90% (Water+0.1% Formic acid)/10% Acetonitrile to 10% (Water+0.1% Formic acid)/90% Acetonitrile in 1.50 minutes; the resulting composition was held for 0.40 min; then the final mobile phase composition; from 10% (Water+0.1% Formic acid)/90% Acetonitrile to 90% (Water+0.1% Formic acid)/10% Acetonitrile in 0.10 minutes. The injection volume was 2 μL with Agilent autosampler injector or 5 μL with Gerstel MPS injector. MS acquisition range and DAD detector were set to 100-800 m/z and 190-400 nm respectively.

### Method 3:

Analyses were carried out on a Thermo Scientific Accucore C18 (50 mm long×2.1 mm I.D., 2.6 μm) at 35° C., with a flow rate of 1.50 mL/min. A gradient elution was performed from 95% (Water+0.1% Formic acid)/5% Acetonitrile to 5% (Water+0.1% Formic acid)/95% Acetonitrile in 1.30 minutes; the resulting composition was held for 0.5 min; then the final mobile phase composition; from 5% (Water+0.1% Formic acid)/95% Acetonitrile to 90% (Water+0.1% Formic acid)/10% Acetonitrile in 0.10 minutes. The injection volume was 1 μL. MS acquisition range and UV detector were set to 100-1000 m/z and 190-400 nm respectively.

### Method 4:

Analyses were carried out on a Thermo Scientific Accucore C18 column PN17126-054630 (50 mm long×4.6 mm I. D.; 2.6 μm particles) at 30° C., with a flow rate of 3 mL/min. A gradient elution was performed from 90% (Water+0.1% Formic acid)/10% Acetonitrile to 5% (Water+0.1% Formic acid)/95% Acetonitrile in 1.50 minutes; the resulting composition was held for 0.90 min; then the final mobile phase composition; from 10% (Water+0.1% Formic acid)/90% Acetonitrile to 90% (Water+0.1% Formic acid)/10% Acetonitrile in 0.10 minutes. The injection volume was 2 μL. MS acquisition range and DAD detector were set to 100-1000 m/z and 200-400 nm respectively.

### Method 5:

Analyses were carried out on a Thermo Scientific Accucore AQ C18 (50 mm long×2.1 mm I.D., 2.6 μm) at 35° C., with a flow rate of 1.50 mL/min. A gradient elution was performed from 95% (Water+0.1% Formic acid)/5% Acetonitrile to 5% (Water+0.1% Formic acid)/95% Acetonitrile in 1.50 minutes; the resulting composition was held for 0.3 min; then the final mobile phase composition; from 5% (Water+0.1% Formic acid)/95% Acetonitrile to 95% (Water+0.1% Formic acid)/5% Acetonitrile in 0.10 minutes. The injection volume was 1 μL. MS acquisition range and UV detector were set to 100-1000 m/z and 190-400 nm respectively.

### Method 6:

Analyses were carried out on a YMC pack ODS-AQ C18 column (50 mm long×4.6 mm I. D.; 3 μm particle size) at 35° C., with a flow rate of 2.6 mL/min. A gradient elution was performed using ISET 2V1.0 Emulated Agilent Pump G1312A V1.0 from 94.51% (Water+0.1% Formic acid)/5.49% Acetonitrile to 5% (Water+0.1% Formic acid)/95% Acetonitrile in 4.8 min; the resulting composition was held for 1.0 min; from 5% (Water+0.1% formic acid)/95% Acetonitrile to 95% (Water+0.1% formic acid)/5% Acetonitrile in 0.2 min. The standard injection volume was 2 μL. Acquisition ranges were set to 190-400 nm for the UV-PDA detector and 100-1000 m/z for the TOF-MS detector.

### Method 7:

Instrument MS: Thermo Scientific FT-MS; Instrument type UHPLC+: Thermo Scientific UltiMate 3000; Column: Waters, HSST3, 2.1×75 mm, C18 1.8 μm; eluent A: 1 L water+0.01% formic acid; eluent B: 1 L acetonitrile+0.01% formic acid; gradient: 0.0 min 10% B→2.5 min 95% B→3.5 min 95% B; oven: 50° C.; flow rate: 0.90 ml/min; UV detection: 210 nm/optimum integration path 210-300 nm.

### Method 8:

Instrument: Waters ACQUITY SQD UPLC System; Column: Waters Acquity UPLC HSS T3 1.8μ 50×1 mm; eluent A: 1 L water+0.25 ml 99% formic acid, eluent B: 1 L acetonitrile+0.25 ml 99% formic acid; gradient: 0.0 min 90% A→1.2 min 5% A→2.0 min 5% A; oven: 50° C.; flow rate: 0.40 ml/min; UV detection: 210 nm.

### Method 9:

Instrument: Waters Single Quad MS System; Instrument Waters UPLC Acquity; Column: Waters BEH C18 1.7μ 50×2.1 mm; eluent A: 1 L water+1.0 mL (25% ammonia)/L, eluent B: 1 L acetonitrile; gradient: 0.0 min 92% A→0.1 min 92% A→1.8 min 5% A→3.5 min 5% A; oven: 50° C.; flow rate: 0.45 mL/min; UV-detection: 210 nm.

### Method 10:

Instrument: Agilent MS Quad 6150; HPLC: Agilent 1290; column: Waters Acquity UPLC HSS T3 1.8 μm 50×2.1 mm; eluent A: 1 L water+0.25 ml 99% formic acid, eluent B: 1 L acetonitrile+0.25 ml 99% formic acid; gradient: 0.0 min 90% A→0.3 min 90% A→1.7 min 5% A→3.0 min 5% A; ofen: 50° C.; flow rate: 1.20 mL/min; UV-detection: 205-305 nm.

### Method 11:

Instrument: Agilent MS Quad 6150; HPLC: Agilent 1290; column: Waters Acquity UPLC HSS T3 1.8 μm 50×2.1 mm; eluent A: 1 L water+0.25 ml 99% formic acid, eluent B: 1 L acetonitrile+0.25 ml 99% formic acid; gradient: 0.0 min 90% A→0.3 min 90% A→1.7 min 5% A→3.0 min 5% A; oven: 50° C.; flow rate: 1.20 ml/min; UV-detection: 205-305 nm.

### Methode 12:

Instrument: Waters ACQUITY SQD UPLC System; column: Waters Acquity UPLC HSS T3 1.8 μm 50×1 mm; eluent A: 1 l water+0.25 ml 99% formic acid, Eluent B: 1 l Acetonitril+0.25 ml 99% formic acid; gradient: 0.0 min 95% A→6.0 min 5% A→7.5 min 5% A; oven: 50° C.; flow rate: 0.35 ml/min; UV-detection: 210 nm.

### Methode 13:

Instrument MS: Waters SQD; Instrument type HPLC: Waters Alliance 2795; Column: Waters, Cortecs, 3.0×30 mm, C18 2.7 μm; eluent A: 1 L water+0.01% formic acid; eluent B: 1 L acetonitrile+0.01% formic acid; gradient: 0.0 min 5% B→1.75 min 95% B→2.35 min 95% B; oven 45° C.: flow rate: 1.75 mL/min; UV detection: 210 nm.

### Methode 14:

Instrument MS: Waters SQD; Instrument HPLC: Waters UPLC; column: Zorbax SB-Aq (Agilent), 50 mm×2.1 mm, 1.8 μm; eluent A: water+0.025% formic acid, eluent B: acetonitrile (ULC)+0.025% formic acid; gradient: 0.0 min 98% A-0.9 min 25% A-1.0 min 5% A-1.4 min 5% A-1.41 min 98% A-1.5 min 98% A; oven: 40° C.; flow: 0.600 ml/min; UV-detection: DAD; 210 nm.

### GC-MS Procedures

### Method A:

Instrument: Thermo Scientific DSQII, Thermo Scientific Trace GC Ultra; column: Restek RTX-35MS, 15 m×200 μm×0.33 μm; constant flow with helium: 1.20 ml/min; oven: 60° C.; Inlet: 220° C.; gradient: 60° C., 30° C./min→300° C. (3.33 min hold).

### Enantiomeric Separation, Preparative Scale:

Method 1 D: Phase: Daicel Chiralpak IF, 5 μm 250 mm×20 mm; eluent: n-heptane/ethanol 50:50; flow: 20 ml/min, temperature: 40° C.; UV-Detection: 220 nm.

Method 2D: Phase: Daicel Chiralpak IE, 5 μm 250 mm×20 mm; eluent: n-heptane/2-propanol 60:40; flow: 15 ml/min, temperature: 50° C.; UV-Detection: 220 nm.

Method 3D: Phase: Daicel Chiralpak IG, 5 μm 250 mm×20 mm; eluent:ethanol 100%; flow: 15 ml/min, temperature: 50° C.; UV-Detection: 220 nm.

Method 4D: Phase: Daicel Chiralpak AD-H, 5 μm 250 mm×20 mm; eluent: n-heptane:ethanol 40:60; flow: 25 ml/min, temperature: 35° C.; UV-Detection: 220 nm.

Method 5D: Phase: Daicel Chiralpak AD-H, 5 μm 250 mm×20 mm; eluent: n-heptane:ethanol 50:50; flow: 25 ml/min, temperature: 30° C.; UV-Detection: 220 nm.

Method 6D: Phase: Daicel Chiralpak IF, 5 μm 250 mm×20 mm; eluent: n-heptane:ethanol 50.50; flow: 15 ml/min, temperature: 40° C.; UV-Detection: 210 nm.

Method 7D: Phase: Daicel Chiralpak IF, 5 μm 250 mm×20 mm, Eluent: n-heptane:ethanol 50:50; flow: 15 ml/min, temperature: 40° C.; UV-Detection: 220 nm.

Method 8D: Phase: Daicel Chiralpak AD-H, 5 μm 250 mm×20 mm, Eluent: i-hexand:ethanol 50:50; flow: 20 ml/min, temperature: 40° C.; UV-Detection: 210 nm.

Method 9D: Phase: Daicel Chiralpak IF, 5 μm 250 mm×20 mm; eluent: n-heptane:ethanol 40:60; flow: 15 ml/min, temperature: 40° C.; UV-Detection: 210 nm.

Method 10D: Phase: Daicel Chiralpak IG, 5 μm 250 mm×20 mm; eluent: n-heptane:ethanol 30:70; flow: 15 ml/min, temperature: 30° C.; UV-Detection: 220 nm.

Method 11D: Phase: Daicel Chiralpak IG, 5 μm 250 mm×20 mm; eluent:ethanol 100%; flow: 15 ml/min, temperature: 50° C.; UV-Detection: 220 nm.

Method 12D: Phase: Daicel Chiralpak IG, 5 μm 250 mm×20 mm; eluent: n-heptane:ethanol 10:90; flow: 15 ml/min, temperature: 50° C.; UV-Detection: 220 nm.

Method 13D: Phase: Daicel Chiralpak IG, 5 μm 250 mm×20 mm, Eluent: ethanol 100%; flow: 15 ml/min, temperature: 50° C.; UV-Detection: 220 nm.

Method 14D: Phase: Daicel Chiralpak IF, 5 μm 250 mm×20 mm, Eluent: ethanol 100%; flow: 15 ml/min, temperature: 50° C.; UV-Detection: 220 nm.

Method 15D: Phase: Daicel Chiralpak IG, 5 μm 250 mm×20 mm; eluent: n-heptane:ethanol 60:40; flow: 15 ml/min, temperature: 50° C.; UV-Detection: 235 nm.

Method 16D: Phase: Daicel Chiralpak IG, 5 μm 250 mm×20 mm; eluent: n-heptane:ethanol 10:90; flow: 15 ml/min, temperature: 30° C.; UV-Detection: 235 nm.

Method 17D: Phase: Daicel Chiralpak IF, 5 μm 250 mm×20 mm; eluent:ethanol 100%; flow: 15 ml/min, temperature: 70° C.; UV-Detection: 220 nm.

### Enantiomeric Separation, Analytical Scale:

Method 1 E: Phase: Chiratek IF-3; eluent: i-hexane/ethanol 50:50; flow: 1 ml/min; UV-Detection: 220 nm.

Method 2E: Phase: Daicel Chiralpak IF, 5 μm 250 mm×4.6 mm; eluent: hexane/2-propanol 30:70; flow: 1.0 ml/min, temperature: 60° C.; UV-Detection: 270 nm.

Method 3E: Phase: Daicel Chiralpak IG, 5 μm 250 mm×4.6 mm; eluent:ethanol 100%; flow: 1.0 ml/min, temperature: 50° C.; UV-Detection: 220 nm.

Method 4E: Phase: Daicel AD, 5 μm 250 mm×4.60 mm; eluent:ethanol 100%; flow: 1.0 ml/min; UV-Detection: 220 nm.

Method 5E: Phase: Daicel Chiralpak AD-H, 5 μm 250 mm×4.6 mm; eluent:ethanol 100%; flow: 1.0 ml/min, UV-Detection: 220 nm.

Method 6E: Phase: Daicel Chiralpak IF, 5 μm 50 mm×4.6 mm; eluent: n-heptane:ethanol 50:50; flow: 1.0 ml/min, Temperature: 25° C.; UV-Detection: 220 nm.

Method 7E: Phase: Daicel Chiralpak IF, 5 μm 50 mm×4.6 mm; eluent: n-heptane:ethanol 50:50; flow: 1.0 ml/min, UV-Detection: 220 nm.

Method 8E: Phase: Chiracel-H AD, 5 μm 250 mm×4.6 mm Eluent: n-heptane:ehanol 50:50; flow: 1.0 ml/min, UV-Detection: 220 nm.

Method 9E: Phase: Daicel Chiralpak IF-3, 5 μm 50 mm×4.6 mm; eluent: n-heptane:ehanol 50:50; flow: 1.0 ml/min, UV-Detection: 220 nm.

Method 10E: Phase: Daicel IG-3, 5 μm 50 mm×4.6 mm; flow: 1.0 ml/min, UV-Detection: 220 nm.

Method 11 E: Phase: Daicel Chiralpak IG-3, 5 μm 250 mm×4.6 mm; eluent:ethanol 100%, flow: 1.0 ml/min, temperature: 50° C.; UV-Detection: 220 nm.

Method 12E: Phase: Daicel Chiralpak IG-3, 5 μm 250 mm×4.6 mm; eluent:ethanol 100%, flow: 1 ml/min, temperature: 50° C.; UV-Detection: 220 nm.

Method 13E: Phase: Daicel Chiralpak IG-3, 5 μm 250 mm×4.6 mm; eluent:ethanol 100%, flow: 1.0 ml/min, temperature: 50° C.; UV-Detection: 220 nm.

Method 14E: Phase: Daicel Chiralpak IF, 5 μm 50 mm×4.60 mm; eluent:ethanol 100%, flow:

1.0 ml/min, UV-Detection: 220 nm.

Method 15E: Phase: Daicel Chiralpak IF, 5 μm 250 mm×4.60 mm; eluent:ethanol 100%, flow: 1.0 ml/min, temperature: 50° C.; UV-Detection: 220 nm.

Method 16E: Phase: Daicel IG-3 3 μm 50 mm×4.60 mm; eluent:ethanol 100%, flow 1.0 ml/min; UV-Detection: 220 nm.

Method 17E: Phase: Daicel Chiralpak IG 5 μm 50 mm×4.60 mm; eluent:ethanol 100%, flow 1.0 ml/min; temperature: 70° C.; UV-Detection: 220 nm.

### Preparative HPLC:

### Method 1f

Column: Chromatorex C18 10 μm; 125×20 mm; Eluent A: water+0.05% trifluoroacetic acid, Eluent B: acetonitrile+0.05% trifluoroacetic acid; Gradient: 10% B→100% B; Flow: 20 ml/min UV-Detection 210 nm.

### Method 2f

Column: Chromatorex C18 10 μm; 125×30 mm; Eluent A: water+0.05% trifluoroacetic acid, Eluent B: acetonitrile+0.05% trifluoroacetic acid; Gradient: 10% B→100% B; Flow: 50 till 100 ml/min UV-Detection 210 nm.

### Method 3f

Instrument: Waters Prep LC/MS System, Column: Phenomenex Kinetex C18 5 μm 100×30 mm; Eluent A: water, Eluent B: acetonitrile, Eluent C: 2% formic acid in Water, Eluent D: acetonitrile/water (80 Vol %/20 Vol %); Flow rate: 80 ml/min; room temperature, UV-detection 200-400 nm; Gradient: Eluent A 0→2 min 63 ml, Eluent B 0→2 min 7 ml, Eluent A 2→10 min from 63 ml till 39 ml and Eluent B from 7 ml till 31 ml, 10→12 min 0 ml Eluent A and 70 ml Eluent B. Eluent C and Eluent D constant flow of 5 ml/min during the complete HPLC run.

### Method 4f

Instrument: Waters Prep LC/MS System, Column: Phenomenex Kinetex C18 5 μm 100×30 mm; Eluent A: water, Eluent B: acetonitrile, Eluent C: 2% formic acid in Water, Eluent D: acetonitrile/water (80 Vol. %/20 Vol %); Flow rate: 80 ml/min; room temperature, UV-detection 200-400 nm; Gradient: Eluent A 0→2 min 55 ml, Eluent B 0→2 min 15 ml, Eluent A 2→10 min from 55 ml till 31 ml and Eluent B from 15 ml till 39 ml, 10→12 min 0 ml Eluent A and 70 ml Eluent B. Eluent C and Eluent D constant flow of 5 ml/min during the complete HPLC run.

### Methode 5f

Instrument: Waters Prep LC/MS System, Column: Phenomenex Kinetex C18 5 μm 100×30 mm; Eluent A: water, Eluent B: acetonitrile, Eluent C: 2% formic acid in Water, Eluent D: acetonitrile/water (80 Vol. %/20 Vol %); Flow rate: 80 ml/min; room temperature, UV-detection 200-400 nm; Gradient: Eluent A 0→2 min 70 ml, Eluent B 0→2 min 0 ml, Eluent A 2→10 min from 70 ml till 55 ml and Eluent B from 0 ml till 15 ml, 10→12 min 0 ml Eluent A and 70 ml Eluent B. Eluent C and Eluent D constant flow of 5 ml/min during the complete HPLC run.

### Experimental Section—Starting Materials and Intermediates

### Intermediate 1

### Benzyl {2-[methoxy(methyl)amino]-2-oxoethyl}carbamate

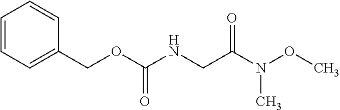 [SMILES: CON(C)C(=O)CNC(=O)OCc1ccccc1]

14.00 mL (110.00 mmol) of isobutyl chloroformate was added to a solution of 15.00 g (71.70 mmol) of N-[(benzyloxy)carbonyl]glycine and 16.00 mL (140.00 mmol) of 4-methylmorpholine in 170 mL of dichloromethane at −15° C. and the mixture was stirred for 1 hour. Then 7.69 g (78.90 mmol) of N,O-dimethylhydroxiamine hydrochloride in 30 mL of dichloromethane was added to the solution at −15° C. and the reaction was further stirred at room temperature for 2 hours. The mixture was quenched with water and extracted with dichloromethane. The combined organic layers were dried over magnesium sulfate, filtered and concentrated. The corresponding residue was purified by flash chromatography on silica gel eluting with dichloromethane/methanol mixtures to afford 11.80 g (65%) of the product as a white powder.

¹H-NMR (300 MHz, DMSO-d₆): δ [ppm]=3.10 (s, 3H), 3.69 (s, 3H), 3.92 (d, 2H), 5.04 (s, 2H), 7.16-7.43 (m, 6H).

LC-MS (Method 3): Rₜ=0.571 min. MS (Mass method 1): m/z=253 (M+H)⁺

### Intermediate 2

### Benzyl (3-methyl-2-oxobutyl)carbamate

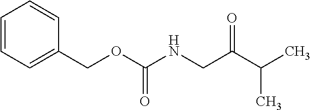 [SMILES: CC(C)C(=O)CNC(=O)OCc1ccccc1]

A solution of 24.00 mL (69.00 mmol) of isopropyl magnesium bromide (2.9M in 2-methyltetrahydrofuran) was slowly added drop wise at 0° C. to a solution of 5.00 g (19.80 mmol) of benzyl {2-[methoxy(methyl)amino]-2-oxoethyl}carbamate in 60 mL of anhydrous tetrahydrofuran under inert atmosphere and the mixture was stirred at room temperature for 10 hours. The reaction was quenched with saturated ammonium chloride(aq.) and the resulting slurry was extracted with ethyl acetate. The combined organic extracts were dried over magnesium sulfate, filtered, concentrated and chromatographed in heptane/ethyl acetate mixtures to afford 3.00 g (64%) of the expected compound as a colourless oil.

¹H-NMR (300 MHz, DMSO-d₆): δ [ppm]=0.97 (d, 6H), 2.60-2.75 (m, 1H), 3.93 (d, 2H), 5.01 (s, 2H), 7.20-7.42 (m, 5H), 7.43 (t, 1H).

LC-MS (Method 3): Rₜ=0.769 min. MS (Mass method 1): m/z=258 (M+Na)⁺

### Intermediate 3

### rac-benzyl [(4-isopropyl-2,5-dioxoimidazolidin-4-yl)methyl]carbamate

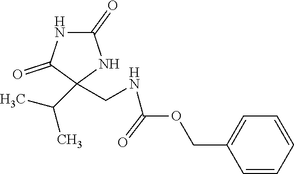 [SMILES: CC(C)C1(CNC(=O)OCc2ccccc2)NC(=O)NC1=O]

A solution of 1.40 g (5.95 mmol) of benzyl (3-methyl-2-oxobutyl)carbamate in 10 mL of ethanol was added to a solution of 0.77 g (11.90 mmol) of potassium cyanide and 2.86 g (29.80 mmol) of ammonium carbonate in 10 mL of water into a pressure flask, the container was sealed and the mixture was stirred at 60° C. for 24 hours. After that time, the solvent was partially removed and the resulting precipitate was filtered off to give 1.43 g (78%) of the product as a withe solid. The compound was used as such in the next step.

¹H-NMR (300 MHz, DMSO-d₆): δ [ppm]=0.80 (d, 3H), 0.89 (d, 3H), 1.85-2.00 (m, 1H), 5.02 (d, 2H), 7.20-7.40 (m, 6H), 7.71 (s, 1H), 10.56 (bp, 1H).

LC-MS (Method 3): Rₜ=0.555 min. MS (Mass method 1): m/z=306 (M+H)⁺

### Intermediate 4

### rac-5-(aminomethyl)-5-(propan-2-yl)imidazolidine-2,4-dione

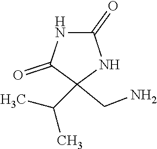

2.85 g (9.33 mmol) of rac-benzyl [(4-isopropyl-2,5-dioxoimidazolidin-4-yl)methyl]carbamate were dissolved in 30 mL of methanol and 0.10 g (0.93 mmol) mmol) of palladium on carbon (Degussa type, 10% loading, wet basis) was added and the mixture was stirred at room temperature under hydrogen atmosphere for 14 hours. The black suspension was filtered through a pad of celite and the filtrates were concentrated under vacuum to give 1.40 g (88%) of the product as a withe solid. The compound was used as such in the next step.

¹H-NMR (300 MHz, DMSO-d₆): δ [ppm]=0.78 (d, 3H), 0.83 (d, 3H), 1.75-1.98 (m, 1H), 7.63 (s, 1H), 10.42 (bp, 1H).

LC-MS (Method 1): Rₜ=0.506 min. MS (Mass method 1): m/z=172 (M+H)⁺

### Intermediate 5

### tert-Butyl {2-[methoxy(methyl)amino]-2-oxoethyl}carbamate

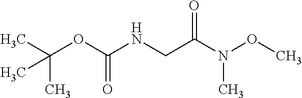

7.30 mL (56.00 mmol) of isobutyl chloroformate was added to a solution of 6.53 g (37.30 mmol) of N-BOC-glycine and 8.20 mL (75.00 mmol) of 4-methylmorpholine in 100 mL of dichloromethane at −15° C. and the mixture was stirred for 1 hour. Then 4.00 g (41.00 mmol) of N,O-dimethylhydroxiamine hydrochloride in 20 mL of dichloromethane was added to the solution at −15° C. and the reaction was further stirred at room temperature for 2 hours. The mixture was quenched with water and extracted with dichloromethane. The combined organic layers were dried over magnesium sulfate, filtered and concentrated. The corresponding residue was purified by flash chromatography on silica gel eluting with dichloromethane/methanol mixtures to afford 4.62 g (57%) of the product as a white powder.

¹H-NMR (300 MHz, DMSO-d₆): δ [ppm]=137 (s, 9H), 3.08 (s, 3H), 3.67 (s, 3H), 3.82 (d, 2H), 6.82 (bp, 1H).

LC-MS (Method 3): Rₜ=0.499 min. MS (Mass method 1): m/z=163 (M−tBu+H)⁺

### Intermediate 6

### rac-tert-butyl (3-methyl-2-oxopent-4-en-1-yl)carbamate

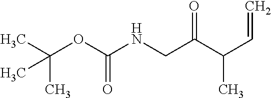

120 mL (58.00 mmol) of 1-methyl-2-propenylmagnesium chloride (0.5M in tetrahydrofuran) were slowly added drop wise at 0° C. to a solution of 3.62 g (16.60 mmol) of tert-butyl {2-[methoxy(methyl)amino]-2-oxoethyl}carbamate in 40 mL of tetrahydrofuran under nitrogen atmosphere and the mixture was stirred at room temperature for 10 hours. The reaction was quenched with saturated ammonium chloride solution in water and the resulting slurry was extracted with acetate. The combined organic extracts were dried over magnesium sulfate, filtered, concentrated and chromatographed in heptane/ethyl acetate mixtures to afford 3.11 g (88%) of the product as a colourless oil.

¹H-NMR (300 MHz, DMSO-d₆): δ [ppm]=1.07 (d, 2H), 1.37 (s, 9H), 1.56 (d, 1H), 3.18 (d, 1H), 3.77 (d, 1H), 3.84 (d, 1H), 5.09-5.20 (m, 1H), 5.45-5.65 (m, 1H), 5.73-5.90 (m, 1H), 6.91-7.05 (m, 1H)

LC-MS (Method 3): Rₜ=0.745 min. MS (Mass method 1): m/z=158 (M−tBu+H)⁺

### Intermediate 7

### rac-tert-butyl {[4-(but-3-en-2-yl)-2,5-dioxoimidazolidin-4-yl]methyl}carbamate

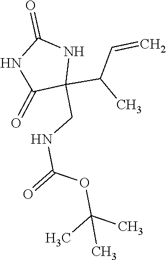 [SMILES: C=CC(C)C1(CNC(=O)OC(C)(C)C)NC(=O)NC1=O]

A solution of 3.10 g (14.50 mmol) of rac-tert-butyl (3-methyl-2-oxopent-4-en-1-yl)carbamate in 20 mL of ethanol was added to a solution of 1.89 g (29.10 mmol) of potassium cyanide and 14.00 g (145.00 mmol) of ammonium carbonate in 20 mL of water into a pressure flask, the container was sealed and the mixture was stirred at 60° C. for 24 hours. After that time, the solvent was partially removed and the resulting precipitate was filtered off to give 2.30 g (56%) of the product as a withe solid. The compound was used as such in the next step.

¹H-NMR (300 MHz, DMSO-d₆): δ [ppm]=0.85-1.25 (m, 1H), 1.36 (s, 9H), 1.50-1.60 (m, 3H), 2.15-2.47 (m, 1H), 3.19 (d, 2H), 4.97-5.27 (m, 1H), 5.50-5.70 (m, 1H), 6.57-6.90 (m, 1H), 7.60 (s, 1H), 10.55 (bp, 1H).

LC-MS (Method 3): Rₜ=0.576 min. MS (Mass method 1): m/z=228 (M−tBu+H)⁺

### Intermediate 8

### rac-5-(aminomethyl)-5-[but-3-en-2-yl]imidazolidine-2,4-dione hydrochloride

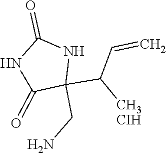

2.63 g (9.28 mmol) of rac-tert-butyl {[4-(but-3-en-2-yl)-2,5-dioxoimidazolidin-4-yl]methyl}carbamate was dissolved in 30 mL of dichloromethane and 12.00 mL (46.00 mmol) of 4M hydrochloric acid in dioxane were added and the resulting suspension was stirred at room temperature for 12 hours. After that time, the solvent was removed by bubbling a stream of nitrogen through the suspension to give 2.00 g (98%) of the product a solid. The compound was used as such in the next step.

¹H-NMR (300 MHz, DMSO-d₆): δ [ppm]=0.85-1.10 (m, 1H), 1.55-1.68 (m, 3H), 2.35-2.48 (m, 1H), 2.84-3.01 (m, 1H), 3.03-3.20 (m, 1H), 5.06-5.30 (m, 1H), 5.60-5.77 (m, 1H), 8.00 (s, 1H), 8.11 (bp, 3H), 10.92-11.09 (m, 1H).

LC-MS (Method 1): Rₜ=0.511 min. MS (Mass method 1): m/z=184 (M+H)⁺

### Intermediate 9

### 1-Azido-3,3-dimethylbutan-2-one

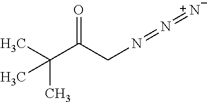

0.78 g (11.98 mmol) of sodium azide were suspended in 30 mL of acetone and 1.65 g (9.21 mmol) of 1-bromopinacolone were added. The resulting mixture was stirred at room temperature for 4 hours. The precipitate was filtered off and discarded and the filtrates were concentrated in vacuo to give 1.15 g (88%) of the product as a pale yellow oil. The compound was used as such in the next step.

¹H-NMR (300 MHz, DMSO-d₆): δ [ppm]=1.10 (s, 9H), 4.38 (s, 2H).

LC-MS (Method 3): Rₜ=0.138 min. MS (Mass method 1): m/z=no ionisation

### Intermediate 10

### rac-5-(azidomethyl)-5-tert-butylimidazolidine-2,4-dione

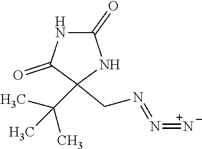

To a stirring solution of 25.52 g (0.26 mol) of ammonium carbonate and 3.79 g (0.07 mol) of ammonium chloride in 30 mL of water previously placed in a sealed tube was added a solution of 2.50 g (0.02 mol) of 1-azido-3,3-dimethylbutan-2-one in 30 mL of ethanol. After 15 min of stirring at room temperature, 5.19 g (0.08 mol) of potassium cyanide were added and the flask was sealed and stirred at 60° C. for 16 hours. After that time, the organic solvent was removed and the resulting precipitate was filtered off and washed with water to give 2.40 g (64%) of the product as a light brown solid. The compound was used as such in the next step.

¹H-NMR (300 MHz, DMSO-d₆): δ [ppm]=0.93 (s, 9H), 3.65 (dd, 2H), 8.13 (s, 1H), 10.72 (s, 1H)

LC-MS (Method 3): Rₜ=0.398 min. MS (Mass method 1): m/z=212 (M+H)⁺

### Intermediate 11

### rac-5-(aminomethyl)-5-tert-butylimidazolidine-2,4-dione

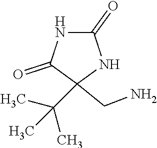

2.40 g (11.40 mmol) of rac-5-(azidomethyl)-5-tert-butylimidazolidine-2,4-dione and 2.98 g (11.40 mmol) of triphenylphosphine were dissolved in 40 mL of tetrahydrofuran and 8 mL of water and the mixture was stirred at 65° C. for 4 h. The solvent was removed and the pure compound was precipitated with dichloromethane/methanol 9:1 to give 1.30 g (61%) of the product as a white powder. The compound was used in the next step without further purification.

¹H-NMR (300 MHz, DMSO-d₆): δ [ppm]=0.91 (s, 9H), 1.22 (bp, 2H), 2.73 (d, 1H), 2.93 (d, 1H), 7.59 (s, 1H), 10.39 (bp, 1H).

LC-MS (Method 3): Rₜ=0.239 min. MS (Mass method 1): m/z=186 (M+H)⁺

### Intermediate 12

### tert-Butyl (3-cyclopropyl-2-oxopropyl)carbamate

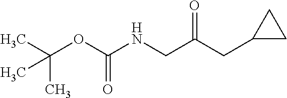

In a flame-dried three-necked round-bottom flask equipped with a constant-pressure addition funnel under nitrogen atmosphere were placed 1.66 g (68.10 mmol) of magnesium turnings and 70 mL of anhydrous tetrahydrofuran was added via syringe. 5.70 mL (59.00 mmol) of cyclopropylmethyl bromide in 10 mL of anhydrous tetrahydrofuran was placed in the funnel and added drop wise to the mixture. Once the addition finished, the reaction was stirred at 65° C. for 1 hour. After that time, the mixture was allowed to stand and the supernatant solution (c.a. 0.842M) was added drop wise to a solution of 5.00 g (22.90 mmol) of tert-butyl {2-[methoxy(methyl)amino]-2-oxoethyl}carbamate in 20 mL of anhydrous tetrahydrofuran at 0° C. under nitrogen atmosphere. The resulting mixture was warmed to room temperature and stirred under the same conditions for 12 hours. The reaction was then concentrated, partitioned between ethyl acetate and aqueous ammonium chloride(sat.) and extracted with ethyl acetate. The combined organic extracts were dried over magnesium sulfate, filtered, concentrated and purified by flash column chromatography on silica gel eluting with heptane/ethyl acetate to give 3.75 g (77%) of the product as a yellowish oil.

¹H-NMR (300 MHz, DMSO-d₆): δ [ppm]=0.80-0.90 (m, 1H), 1.38 (s, 9H), 2.15-2.28 (m, 2H), 2.43-2.50 (m, 1H), 3.73 (d, 2H), 4.88-5.08 (m, 2H), 5.70-5.88 (m, 1H), 7.04 (t, 1H). LC-MS (Method 3): Rₜ=0.725 min. MS (Mass method 1): m/z=159 (M−tBu+H)⁺

### Intermediate 13

### rac-tert-butyl {[4-(cyclopropylmethyl)-2,5-dioxoimidazolidin-4-yl]methyl}carbamate

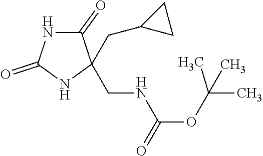 [SMILES: CC(C)(C)OC(=O)NCC1(CC2CC2)NC(=O)NC1=O]

A solution of 3.75 g (17.60 mmol) of tert-butyl (3-cyclopropyl-2-oxopropyl)carbamate in 20 mL of ethanol was added to a solution of 2.29 g (35.20 mmol) of potassium cyanide and 16.90 g (176.00 mmol) of ammonium carbonate in 30 mL of water into a pressure flask, the container was sealed and the mixture was stirred at 60° C. for 12 hours. The organic solvent was removed in vacuo and the resulting precipitate was collected by filtration and washed with water. 2.86 g (57%) of the product were obtained as an off-white powder. The compound was used as such in the next step.

¹H-NMR (300 MHz, DMSO-d₆): δ [ppm]=1.37 (s, 9H), 1.50-1.73 (m, 2H), 1.79-1.90 (m, 1H), 1.95-2.10 (m, 1H), 3.17 (d, 2H), 4.80-5.05 (m, 2H), 5.65-5.85 (m, 1H), 6.83 (t, 1H), 7.59 (s, 1H).

LC-MS (Method 3): Rₜ=0.542 min. MS (Mass method 1): m/z=228 (M−tBu+H)⁺

### Intermediate 14

### rac-5-(aminomethyl)-5-(cyclopropylmethyl)imidazolidine-2,4-dione hydrochloride

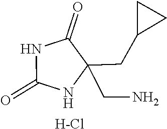

2.75 g (9.71 mmol) of rac-tert-butyl {[4-(cyclopropylmethyl)-2,5-dioxoimidazolidin-4-yl]methyl}carbamate were dissolved in 55 mL of dichloromethane and 12.00 mL (49.00 mmol) of 4N hydrochloric acid in dioxane was added. The resulting yellowish solution was allowed to stir at room temperature for 12 hours. The solvent was removed under reduced pressure and the resulting precipitate was filtered off, washed with dichloromethane, ethyl acetate and diethyl ether to give 1.92 g (89%) of the product as a white powder. The compound was used as such in the next step

¹H-NMR (300 MHz, DMSO-d₆): δ [ppm]=1.70-1.93 (m, 3H), 2.00-2.10 (m, 1H), 2.95 (d, 1H), 3.11 (d, 1H), 4.93-5.10 (m, 2H), 5.69-5.84 (m, 1H), 8.00 (s, 1H), 8.22 (bp, 1H), 11.02 (s, 1H).

LC-MS (Method 1): Rₜ=0.500 min. MS (Mass method 1): m/z=184 (M+H)⁺

### Intermediate 15

### Benzyl (3-cyclopropyl-2-oxopropyl)carbamate

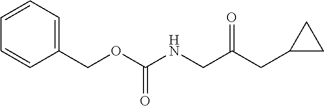 [SMILES: O=C(CNC(=O)OCc1ccccc1)CC1CC1]

In a flame-dried three-necked round-bottom flask equipped with a constant-pressure addition funnel under nitrogen atmosphere were placed 1.66 g (68.10 mmol) of magnesium turnings aid 70 mL of anhydrous tetrahydrofuran was added via syringe. 5.70 mL (59.00 mmol) of cyclopropylmethyl bromide in 10 mL of anhydrous tetrahydrofuran was placed in the funnel and added drop wise to the mixture. Once the addition finished, the reaction was stirred at 65° C. for 1 hour. After that time, the mixture was allowed to stand and the supernatant solution (c.a. 0.842M) was added drop wise to a solution of 9.10 g (20.00 mmol) of benzyl {2-[methoxy(methyl)amino]-2-oxoethyl}carbamate in 35 mL of anhydrous tetrahydrofuran at 0° C. under nitrogen atmosphere. The resulting mixture was warmed to room temperature and stirred under the same conditions for 12 hours. The reaction was then concentrated, partitioned between ethyl acetate and aqueous ammonium chloride(sat.) and extracted with ethyl acetate. The combined organic extracts were dried over magnesium sulfate, filtered, concentrated and purified by flash column chromatography on silica gel eluting with heptane/ethyl acetate to give 4.62 g (94%) of the product as a yellowish oil.

¹H-NMR (300 MHz, DMSO-d₆): δ [ppm]=2.15-2.30 (m, 2H), 3.86 (d, 2H), 4.93-5.00 (m, 2H), 5.04 (s, 2H), 5.70-5.90 (m, 1H), 7.25-7.40 (m, 5H), 7.50 (t, 1H).

LC-MS (Method 3): Rₜ=0.769 min. MS (Mass method 1): m/z=248 (M+H)⁺

### Intermediate 16

### rac-Benzyl {[4-(cyclopropylmethyl)-2,5-dioxoimidazolidin-4-yl]methyl}carbamate

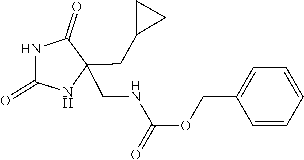 [SMILES: O=C1NC(=O)C(CNC(=O)OCc2ccccc2)(CC2CC2)N1]

A solution of 4.60 g (18.60 mmol) of benzyl (3-cyclopropyl-2-oxopropyl)carbamate in 20 mL of ethanol was added to a solution of 2.42 g (37.20 mmol) of potassium cyanide and 17.87 g (186.01 mmol) of ammonium carbonate in 20 mL of water into a pressure flask, the container was sealed and the mixture was stirred at 60° C. for 12 hours. After that time, the organic solvent was removed in vacuo and the resulting precipitate was filtered off, washed with cold water and dried under reduced pressure to give 4.02 g (68%) of the product as white needles.

¹H-NMR (300 MHz, DMSO-d₆): δ [ppm]=1.58-1.72 (m, 2H), 1.79-1.89 (m, 1H), 1.90-2.07 (m, 1H), 3.25 (t, 2H), 4.90-5.10 (m, 5H), 5.65-5.88 (m, 1H), 7.25-7.45 (m, 5H), 7.72 (s, 1H).

LC-MS (Method 3): Rₜ=0.606 min. MS (Mass method 1): m/z=318 (M+H)⁺

### Intermediate 17

### rac-5-(aminomethyl)-5-butylimidazolidine-2,4-dione

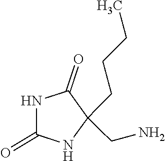

4.80 g (15.10 mmol) of rac-Benzyl {[4-(cyclopropylmethyl)-2,5-dioxoimidazolidin-4-yl]methyl}carbamate was dissolved in 40 mL of methanol and 0.16 g (1.51 mmol) of palladium on carbon (Degussa type, 10% loading, wet basis) was added allowing the suspension to stir at room temperature under hydrogen atmosphere for 12 hours. After that time, the black slurry was filtered through a pad of celite and the filtrates were concentrated under vacuum to afford 1.42 g (50%) of the product as a white powder. The compound was used as such in the next step.

¹H-NMR (300 MHz, DMSO-d₆): δ [ppm]=0.80-0.88 (m, 4H), 1.00-1.10 (m, 1H), 1.17-1.29 (m, 4H), 1.40-1.51 (m, 2H), 2.60 (d, 1H), 2.71 (d, 1H), 7.63 (s, 1H).

LC-MS (Method 1): Rₜ=0.760 min. MS (Mass method 1): m/z=186 (M+H)⁺

### Intermediate 18

### tert-Butyl (2-oxopentyl)carbamate

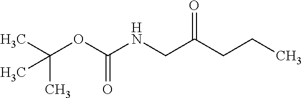

6.90 mL (14.00 mmol) of propylmagnesium chloride (2M in diethyl ether) was slowly added drop wise at 0° C. to a solution of 1.50 g (6.87 mmol) of tert-butyl {2-[methoxy(methyl)amino]-2-oxoethyl}carbamate in 10 mL of anhydrous tetrahydrofuran under nitrogen atmosphere and the mixture was stirred at room temperature for 10 hours. The reaction was quenched with saturated ammonium chloride(aq.) and the resulting slurry was extracted with ethyl acetate. The combined organic extracts were dried over magnesium sulfate, filtered, concentrated and chromatographed in heptane/ethyl acetate mixtures (potassium permanganate stain required) to afford 0.55 g (40%) of the expected compound as a colourless oil.

¹H-NMR (300 MHz, DMSO-d₆): δ [ppm]=0.84 (t, 3H), 1.38 (s, 9H), 1.47 (q, 2H), 2.35 (t, 2H), 3.71 (d, 2H), 7.01 (t, 1H).

LC-MS (Method 3): Rₜ=0.682 min. MS (Mass method 1): m/z=224 (M+Na)⁺

### Intermediate 19

### rac-tert-Butyl {[2,5-dioxo-4-propylimidazolidin-4-yl]methyl}carbamate

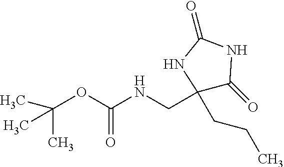 [SMILES: CCCC1(CNC(=O)OC(C)(C)C)NC(=O)NC1=O]

To a stirring solution of 3.94 g (41.00 mmol) of ammonium carbonate and 0.73 g (13.70 mmol) of ammonium chloride in 10 mL of water placed into a sealed flask, was added 0.55 g (2.73 mmol) of tert-butyl (2-oxopentyl)carbamate in 10 mL of ethanol. After 15 min, 0.80 g (12.30 mmol) of potassium cyanide was added and the mixture was heated at 60° C. for 16 hours. The organic solvent was removed and the resulting aqueous phase was allowed to stand at 0° C. for 12 hours. A white precipitate appeared after that time, which was collected by filtration giving 0.60 g (81%) of the product as a white crystalline solid. The compound was used as such in the next step.

¹H-NMR (300 MHz, DMSO-d₆): δ [ppm]=0.84 (t, 3H), 0.99-1.10 (m, 1H), 1.23-1.33 (m, 1H), 1.36 (s, 9H), 1.40-1.55 (m, 2H), 3.12 (d, 2H), 6.78 (bp, 1H), 7.55 (s, 1H), 10.55 (bp, 1H).

LC-MS (Method 3): Rₜ=0.513 min. MS (Mass method 1): m/z=272 (M+H)⁺

### Intermediate 20

### rac-5-(aminomethyl)-5-propylimidazolidine-2,4-dione hydrochloride

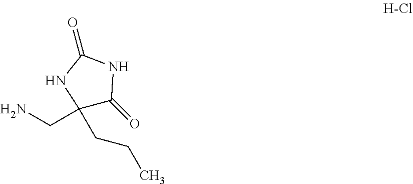

2.80 mL (11.00 mmol) of 4M hydrochloric acid in dioxane were added to a solution of 0.60 g (2.21 mmol) of rac-tert-Butyl {[2,5-dioxo-4-propylimidazolidin-4-yl]methyl}carbamate in 20 mL of dichloromethane and the mixture was stirred at room temperature for 72 hours. The resulting precipitate was filtered off and washed with dichloromethane, ethyl acetate and diethyl ether to afford 0.45 g (98%) of the product as a white powder. The compound was used in the next step without further purification.

¹H-NMR (300 MHz, DMSO-d₆): δ [ppm]=0.89 (t, 3H), 1.00-1.16 (m, 1H), 1.20-1.37 (m, 1H), 1.50-1.68 (m, 2H), 2.90 (d, 1H), 3.08 (d, 1H), 7.94 (s, 1H), 8.09 (bp, 3H), 10.99 (bp, 1H).

LC-MS (Method 3): Rₜ=0.122 min. MS (Mass method 1): m/z=172 (M+H)⁺

### Intermediate 21

### tert-Butyl (2-oxopent-4-en-1-yl)carbamate

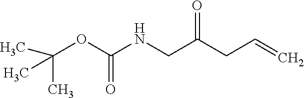

To a solution of 1.50 g (6.87 mmol) of tert-butyl {2-[methoxy(methyl)amino]-2-oxoethyl}carbamate in 20 mL of anhydrous tetrahydrofuran under inert atmosphere at 0° C. was added drop wise 17.00 mL (17.00 mmol) of allylmagnesium bromide solution (1M in tetrahydrofuran) and the mixture was stirred at room temperature for 12 hours. The solvent was partially removed then the mixture was diluted with ethyl acetate and quenched with saturated ammonium chloride aqueous solution. The organic extract was isolated, dried over magnesium sulfate, filtered and concentrated to afford a residue, which was purified by flash column chromatography on silica gel eluting with heptane/ethyl acetate mixtures to give 1.20 g (87%) of the product as a yellowish oil.

LC-MS (Method 3): Rₜ=0.623 min. MS (Mass method 1): m/z=222 (M+Na)⁺

### Intermediate 22

### rac-tert-Butyl {[2,5-dioxo-4-(prop-2-en-1-yl)imidazolidin-4-yl]methyl}carbamate

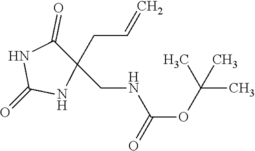 [SMILES: C=CCC1(CNC(=O)OC(C)(C)C)NC(=O)NC1=O]

5.79 g (60.20 mmol) of ammonium carbonate was placed into a sealed flask and 10 mL of water were added. To this mixture, was added a solution of 1.20 g (6.02 mmol) of tert-butyl (2-oxopent-4-en-1-yl)carbamate in 10 mL of ethanol. After 15 min of stirring, 0.78 g (12.00 mmol) of potassium cyanide were added, the flask was sealed and the reaction was heated up to 60° C. for 16 hours. The organic solvent was removed and the resulting aqueous phase was allowed to stand at 0° C. for 12 hours. A white precipitate appeared after that time, which was collected by filtration giving 0.44 g (27%) of the product as a white crystalline solid. The compound was used as such in the next step.

¹H-NMR (300 MHz, DMSO-d₆): δ [ppm]=1.36 (s, 9H), 2.28 (d, 2H), 3.17 (d, 2H), 5.05-5.17 (m, 2H), 5.50-5.70 (m, 1H), 6.83 (t, 1H), 7.62 (s, 1H), 10.57 (s, 1H).

LC-MS (Method 3): Rₜ=0.479 min. MS (Mass method 1): m/z=214 (M−tBu+H)⁺

### Intermediate 23

### rac-5-(aminomethyl)-5-(prop-2-en-1-yl)imidazolidine-2,4-dione hydrochloride

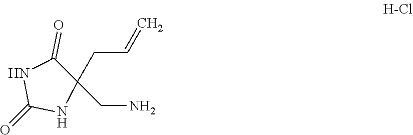

0.44 g (1.64 mmol) of rac-tert-Butyl {[2,5-dioxo-4-(prop-2-en-1-yl)imidazolidin-4-yl]methyl}carbamate were dissolved in 15 mL of dichloromethane and 2.10 mL (8.20 mmol) of 4M hydrochloric acid in dioxane was added, allowing the resulting yellowish solution to stir at room temperature for 12 hours. The solvent was then partially removed by bubbling nitrogen through the solution and the resulting precipitate was filtered off and washed with dichloromethane, ethyl acetate and diethyl ether giving 0.18 g (54%) of the product as an off-white powder.

¹H-NMR (300 MHz, DMSO-d₆): δ [ppm]=2.41 (d, 2H), 2.93 (d, 1H), 3.12 (d, 1H), 5.18 (d, 2H), 5.50-5.70 (m, 1H), 8.00 (s, 1H), 8.21 (bp, 3H), 10.98 (bp, 1H).

### Intermediate 24

### tert-Butyl (2-cyclopentyl-2-oxoethyl)carbamate

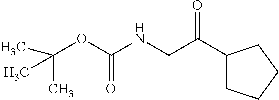 [SMILES: CC(C)(C)OC(=O)NCC(=O)C1CCCC1]

To a solution of 2.20 g (10.10 mmol) of tert-butyl {2-[methoxy(methyl)amino]-2-oxoethyl}carbamate in 10 mL of anhydrous tetrahydrofuran under inert atmosphere at 0° C. was added dropwise 13.00 mL (25.00 mmol) of cyclopentylmagnesium chloride solution (2M in tetrahydrofuran) and the reaction was stirred at room temperature for 12 hours. The solvent was partially removed and the mixture was diluted with ethyl acetate and quenched with saturated ammonium chloride(aq.). The organic layer was isolated, dried over magnesium sulfate, filtered and concentrated to afford a residue, which was purified by flash column chromatography on silica gel eluting with heptane/ethyl acetate to give 0.58 g (25%) of the product as a yellowish oil.

¹H-NMR (300 MHz, DMSO-d₆): δ [ppm]=1.37 (s, 9H), 1.49-1.67 (m, 6H), 1.68-1.84 (m, 2H), 2.85-3.00 (m, 1H), 3.80 (d, 2H), 6.98 (t, 1H).

LC-MS (Method 3): Rₜ=0.795 min. MS (Mass method 1): m/z=172 (M−tBu+H)⁺

### Intermediate 25

### rac-tert-Butyl {[4-cyclopentyl-2,5-dioxoimidazolidin-4-yl]methyl}carbamate

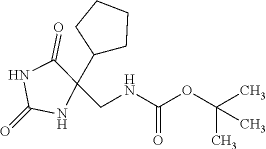 [SMILES: CC(C)(C)OC(=O)NCC1(C2CCCC2)NC(=O)NC1=O]

A solution of 0.58 g (2.56 mmol) of tert-butyl (2-cyclopentyl-2-oxoethyl)carbamate in 5 mL of ethanol was added to another solution of 0.33 g (5.12 mmol) of potassium cyanide and 2.46 g (25.60 mmol) of ammonium carbonate in 5 mL of water into a pressure flask, the container was sealed and the mixture was stirred at 60° C. for 12 hours. The ethanol was removed under reduced pressure and the resulting precipitate was collected by filtration, washed with water aid dried in vacuo to give 0.46 g (60%) of the product as white solid.

¹H-NMR (300 MHz, DMSO-d₆): δ [ppm]=1.09-1.31 (m, 2H), 1.36 (s, 9H), 1.40-1.60 (m, 5H), 1.61-1.75 (m, 1H), 2.01-2.16 (m, 1H), 3.20-3.25 (m, 2H), 6.74 (t, 1H), 7.61 (s, 1H), 10.53 (bp, 1H)

LC-MS (Method 3): Rₜ=0.601 min. MS (Mass method 1): m/z=320 (M+Na)⁺

### Intermediate 26

### rac-5-(aminomethyl)-5-cyclopentylimidazolidine-2,4-dione hydrochloride

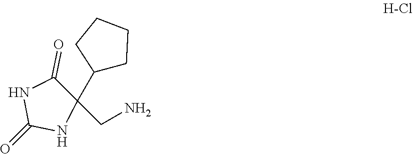 [SMILES: NCC1(C2CCCC2)NC(=O)NC1=O]

1.90 mL (7.70 mmol) of 4N hydrochloric acid in dioxane were added to a solution of 0.46 g (1.55 mmol) of rac-tert-Butyl {[4-cyclopentyl-2,5-dioxoimidazolidin-4-yl]methyl}carbamate in 10 mL of dichloromethane and the mixture was allowed to stir at room temperature for 16 hours. The resulting precipitate was collected by filtration and washed with dichloromethane, ethyl acetate and diethyl ether to give 0.32 g (88%) of the product as a yellow sticky solid. The compound was used as such in the next step.

¹H-NMR (300 MHz, DMSO-d₆): δ [ppm]=1.13-1.38 (m, 2H), 1.40-1.63 (m, 5H), 1.65-1.77 (m, 1H), 2.10-2.25 (m, 1H), 2.95 (d, 1H), 3.12 (d, 1H), 8.09 (s, 1H), 8.20 (bp, 3H), 10.99 (bp, 1H).

LC-MS (Method 1): Rₜ=0.801 min. MS (Mass method 1): m/z=198 (M+H)⁺

### Intermediate 27

### 2-Bromo-1-cyclobutylethan-1-one

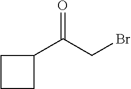

1.31 mL (25.47 mmol) of bromine were added drop wise to a solution of 2.50 g (25.47 mmol) of cyclobutyl methyl ketone in 25 mL of methanol at 0° C. and the solution was then stirred for 1 hour under the same conditions. The solvent was removed under vacuum and the mixture was partitioned between ethyl acetate and water. The organic layer was isolated, dried over magnesium sulfate, filtered and concentrated to afford 3.80 g (84%) of the product as a dark oil. The compound was used as such in the next step.

¹H-NMR (300 MHz, DMSO-d₆): δ [ppm] 1.62-1.81 (m, 1H), 1.84-2.02 (m, 1H), 2.05-2.20 (m, 4H), 3.42-3.58 (m, 1H), 4.51 (s, 2H).

LC-MS (Method 1): Rₜ=2.665 min. MS (Mass method 1): m/z=no ionisation.

### Intermediate 28

### 2-Azido-1-cyclobutylethan-1-one

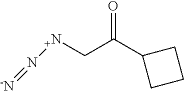

0.75 g (4.24 mmol) of 2-bromo-1-cyclobutylethan-1-one were added to a suspension of 0.36 g (5.51 mmol) of sodium azide in 20 mL of acetone and the mixture was stirred at room temperature for 16 hours. The resulting precipitate was filtered and the filtrate concentrated to give 0.43 g (73%) of the product as a pale orange oil. The compound was used as such in the next step.

¹H-NMR (300 MHz, DMSO-d₆): δ [ppm]=01.68-1.83 (m, 1H), 1.84-2.01 (m, 1H), 2.02-2.22 (m, 4H), 3.22-3.32 (m, 1H), 4.17 (, 1H).

LC-MS (Method 3): Rₜ=0.570 min. MS (Mass method 1): m/z=no ionisation.

### Intermediate 29

### rac-5-(azidomethyl)-5-cyclobutylimidazolidine-2,4-dione

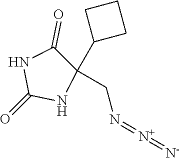 [SMILES: [N-]=[N+]=NCC1(C2CCC2)NC(=O)NC1=O]

A solution of 0.43 g (3.10 mmol) of 2-azido-1-cyclobutylethan-1-one in 5 mL of ethanol was added to a solution of 0.41 g (6.21 mmol) of potassium cyanide and 2.98 g (31.00 mmol) of ammonium carbonate in 5 mL water into a pressure flask. The container was sealed and the mixture was stirred at 60° C. for 12 hours. The ethanolic fraction was removed under reduced pressure and the mixture was extracted with ethyl acetate. The combined organic extracts were dried over magnesium sulfate, filtered and concentrated and the resulting precipitate was filtered off, washed with dichloromethane and dried in vacuo to give 0.23 g (35%) of the product as an orange solid.

¹H-NMR (300 MHz, DMSO-d₆): δ [ppm]=1.58-1.95 (m, 6H), 2.52-2.65 (m, 1H), 3.39 (s, 2H), 8.26 (s, 1H), 10.76 (bp, 1H).

### Intermediate 30

### rac-5-(aminomethyl)-5-cyclobutylimidazolidine-2,4-dione

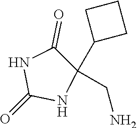 [SMILES: NCC1(C2CCC2)NC(=O)NC1=O]

230 mg (1.10 mmol) of rac-5-(azidomethyl)-5-cyclobutylimidazolidine-2,4-dione were dissolved in 15 mL of methanol in a round-bottom flask with a stirrer then the system was purged with nitrogen. 23 mg (0.22 mmol) of palladium on carbon (Degussa type, 10% loading, wet basis) was added the mixture was stirred at room temperature under hydrogen atmosphere for 12 hours. The black suspension was filtered through a pad of celite and the filtrates were concentrated to afford a pale-orange solid, which was washed with dichloromethane/methanol (9:1) give 180 mg (90%) of the product as white crystals.

¹H-NMR (300 MHz, DMSO-d₆): δ [ppm]=1.59-1.91 (m, 6H), 2.53-2.59 (m, 1H), 2.64 (d, 2H), 7.82 (s, 1H), 10.51 (bp, 1H).

LC-MS (Method 3): Rₜ=0.119 min. MS (Mass method 1): m/z=184 (M+H)⁺

### Intermediate 31

### tert-Butyl (2-cyclohexyl-2-oxoethyl)carbamate

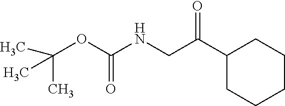 [SMILES: CC(C)(C)OC(=O)NCC(=O)C1CCCCC1]

38.00 mL (38.00 mmol) of cyclohexylmagnesium chloride (1M is tetrahydrofuran) were added drop wise to a solution of 3.30 g (15.10 mmol) of tert-butyl {2-[methoxy(methyl)amino]-2-oxoethyl}carbamate in 40 mL of anhydrous tetrahydrofuran at 0° C. the reaction was allowed to stir at room temperature for 12 hours. The solvent was partially removed, the mixture was diluted with ethyl acetate and the reaction was quenched with saturated ammonium chloride(aq.). The organic extract was isolated, dried over magnesium sulfate, filtered and concentrated to afford a residue, which was purified by flash column chromatography on silica gel eluting with heptane/ethyl acetate to give 1.70 g (47%) of the product as a yellowish oil.

¹H-NMR (300 MHz, DMSO-d₆): δ [ppm]=1.11-1.27 (m, 5H), 1.37 (s, 9H), 1.56-1.78 (m, 5H), 2.37-2.48 (m, 1H), 3.80 (d, 2H), 6.93 (t, 1H).

LC-MS (Method 3): Rₜ=0.868 min. MS (Mass method 1): m/z=142 (M−tBu+H)⁺

### Intermediate 32

### rac-tert-Butyl {[4-cyclohexyl-2,5-dioxoimidazolidin-4-yl]methyl}carbamate

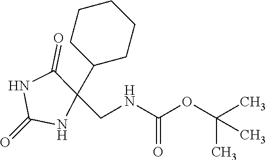 [SMILES: CC(C)(C)OC(=O)NCC1(C2CCCCC2)NC(=O)NC1=O]

A solution of 1.70 g (7.04 mmol) of tert-butyl (2-cyclohexyl-2-oxoethyl)carbamate in 10 mL of ethanol was added to a solution of 0.92 g (14.10 mmol) of potassium cyanide and 6.77 g (70.40 mmol) of ammonium carbonate in 10 mL water into a pressure flask. The container was sealed and the mixture was stirred at 60° C. for 12 hours. The ethanolic fraction was removed under reduced pressure and the resulting precipitate was collected by filtration, washed with water aid dried to give 1.68 g (77%) of the product as a white solid.

¹H-NMR (300 MHz, DMSO-d₆): δ [ppm]=0.88-1.25 (m, 5H), 1.36 (s, 9H), 1.39-1.49 (m, 1H), 1.51-1.78 (m, 5H), 3.13-3.31 (m, 2H), 6.65 (t, 1H), 7.57 (s, 1H), 10.52 (s, 1H).

LC-MS (Method 3): Rₜ=0.659 min. MS (Mass method 1): m/z=256 (M−tBu+H)⁺

### Intermediate 33

### rac-5-(aminomethyl)-5-cyclohexylimidazolidine-2,4-dione hydrochloride

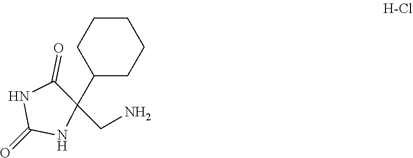 [SMILES: NCC1(C2CCCCC2)NC(=O)NC1=O]

6.70 mL (27.00 mmol) of 4N hydrochloric acid in dioxane were added to a solution of 1.68 g (5.40 mmol) of rac-tert-Butyl {[4-cyclohexyl-2,5-dioxoimidazolidin-4-yl]methyl}carbamate in 30 mL of dichloromethane and the mixture was allowed to stir at room temperature for 16 hours. The resulting precipitate was collected by filtration and washed with dichloromethane, ethyl acetate and diethyl ether to give 1.33 g (99%) of the product as a yellow sticky solid. The compound was used as such in the next step.

¹H-NMR (300 MHz, DMSO-d₆): δ [ppm]=0.90-1.29 (m, 5H), 1.38-1.52 (m, 1H), 1.54-1.81 (m, 5H), 3.06 (1, 2H), 8.01 (s, 1H), 8.14 (bp, 3H), 10.96 (bp, 1H).

LC-MS (Method 1): Rₜ=1.152 min. MS (Mass method 1): m/z=212 (M+H)⁺

### Intermediate 34

### rac-Potassium 3-ethoxy-2-methyl-3-oxopropanoate

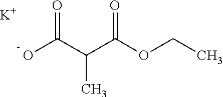

A solution of 1.84 g (27.90 mmol) of potassium hydroxide in 50 mL of absolute ethanol was added to a stirring solution of 5.00 mL (29.00 mmol) of diethyl methylmalonate in 20 mL of absolute ethanol and the mixture was refluxed for 2 hour. After completion of the reaction (as judged by TLC), the mixture was cooled down to room temperature and the solvent was concentrated to dryness to give an oil which crystallised upon standing. The resulting crystals were washed with diisoporpyl ether to afford 4.90 g (91%) of the product as a white crystalline solid.

¹H-NMR (300 MHz, DMSO-d₆): δ [ppm]=1.07-1.16 (m, 6H), 2.99 (q, 1H), 3.97 (q, 2H).

### Intermediate 35

### rac-Ethyl 4-[(tert-butoxycarbonyl)amino]-2-methyl-3-oxobutanoate

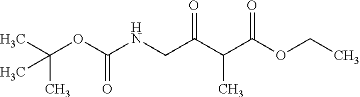 [SMILES: CCOC(=O)C(C)C(=O)CNC(=O)OC(C)(C)C]

1.58 g (9.77 mmol) of 1,1′-carbonyldiimidazole was added to a solution of 1.43 g (8.14 mmol) of N-(tert-butoxycarbonyl)glycine in 20 mL of anhydrous tetrahydrofuran under nitrogen atmosphere and the mixture was stirred at room temperature for 2 hours. Then, a solid mixture of 1.04 g (7.98 mmol) of cobalt(II) dichloride and 2.25 g (12.20 mmol) of potassium rac-3-ethoxy-2-methyl-3-oxopropanoate was incorporated and the reaction was further stirred under the same conditions for 24 hours. The resulting deep blue slurry was quenched with 1N hydrochloric add provoking a colour change from deep blue to pale red. Ethyl acetate was then added and the mixture was washed with 1N hydrochloric acid, aqueous sodium hydrogen carbonate(sat.) aid water. The organic layer was dried over magnesium sulfate, filtered, concentrated and chromatographed in heptane/ethyl acetate mixtures (potassium permanganate stain required) to give 1.00 g (47%) of the product as a colourless oil.

¹H-NMR (300 MHz, DMSO-d₆): δ [ppm]=1.13-1.25 (m, 6H), 1.37 (s, 9H), 3.76 (q, 1H), 3.90 (d, 2H), 4.09 (q, 2H), 7.11 (t, 1H).

LC-MS (Method 3): Rₜ=0.713 min. MS (Mass method 1): m/z=204 (M−tBu+H)⁺

### Intermediate 36

### rac-Ethyl 2-[4-{[(tert-butoxycarbonyl)amino]methyl}-2,5-dioxoimidazolidin-4-yl]propanoate

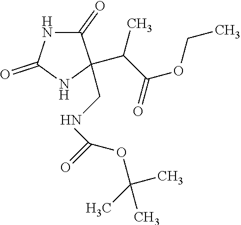 [SMILES: CCOC(=O)C(C)C1(CNC(=O)OC(C)(C)C)NC(=O)NC1=O]

A solution of 1.00 g (3.86 mmol) of ethyl rac-4-[(tert-butoxycarbonyl)amino]-2-methyl-3-oxobutanoate in 15 mL of ethanol was added to a solution of 1.13 g (17.40 mmol) of potassium cyanide and 5.56 g (57.80 mmol) of ammonium carbonate in 15 mL water into a pressure flask. The container was sealed and the mixture was stirred at 60° C. for 12 hours. The ethanolic fraction was removed under reduced pressure and the resulting precipitate was collected by filtration, washed with water and dried to give 0.78 g (62%) of the product as a white solid.

¹H-NMR (300 MHz, DMSO-d₆): δ [ppm]=0.76-0.89 (m, 1.5H), 1.03-1.23 (m, 5.5H), 1.36 (s, 9H), 2.70 (q, 0.5H), 2.85 (q, 0.5H), 3.22 (d, 2H), 3.91-4.11 (m, 2H), 6.51-6.82 (m, 1H), 7.62 (s, 1H), 10.58 (bp, 1H).

LC-MS (Method 3): Rₜ=0.520, 0.566 min (split peak). MS (Mass method 1): m/z=274 (M−tBu+H)⁺

### Intermediate 37

### rac-tert-Butyl ({4-[1-hydroxypropan-2-yl]-2,5-dioxoimidazolidin-4-yl}methyl)carbamate

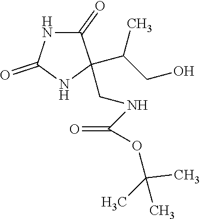 [SMILES: CC(CO)C1(CNC(=O)OC(C)(C)C)NC(=O)NC1=O]

293 mg (7.76 mmol) of sodium borohydride were added to a solution of 730 mg (2.22 mmol) of rac-Ethyl 2-[4-{[(tert-butoxycarbonyl)amino]methyl}-2,5-dioxoimidazolidin-4-yl]propanoate in 29 mL of ethanol at 40° C. for 16 hours. After that time, more sodium borohydride was added (293 mg) and the reaction was continued for 16 additional hours. The mixture was concentrated in vacuo, diluted with ethyl acetate and partitioned between ethyl acetate and water. The aqueous layer was concentrated to dryness to afford a sticky pale-yellow foam. This foam was dissolved in ethanol provoking the precipitation of the most part of the salts presents. The resulting precipitate was filtered off and thoroughly washed with ethanol. The filtrates were concentrated to afford 680 mg (100%) of the product as a pale-yellow solid.

¹H-NMR (300 MHz, DMSO-d₆): δ [ppm]=0.74 (d, 3H), 1.36 (s, 9H), 1.60 (bp, 2H), 1.69-1.81 (m, 1H), 2.98 (d, 1H), 3.12-3.28 (m, 2H), 4.62 (s, 1H), 5.66 (s, 1H), 5.96 (s, 1H).

LC-MS (Method 3): Rₜ=0.346 min. MS (Mass method 1): m/z=232 (M−tBu+H)⁺

### Intermediate 38

### rac-5-(aminomethyl)-5-[1-hydroxypropan-2-yl]imidazolidine-2,4-dione hydrochloride

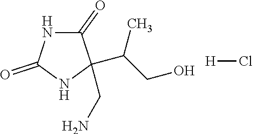

3.00 mL (12.00 mmol) of 4N hydrochloric acid in dioxane were added to a solution of 0.68 g (2.37 mmol) of rac-tert-Butyl ({4-[1-hydroxypropan-2-yl]-2,5-dioxoimidazolidin-4-yl}methyl)carbamate in 15 mL of dichloromethane and the mixture was allowed to stir at room temperature for 16 hours. The resulting precipitate was collected by filtration and washed with dichloromethane, ethyl acetate and diethyl ether to give 0.44 g (83%) of the product as a yellow sticky solid. The compound was used as such in the next step.

¹H-NMR (300 MHz, DMSO-d₆): δ [ppm]=0.80 (d, 3H), 1.98-2.06 (m, 1H), 3.06-3.15 (m, 1H), 3.21-3.30 (m, 1H), 3.23-3.40 (m, 2H), 7.99 (s, 1H), 8.25 (bp, 3H), 10.98 (s, 1H).

LC-MS (Method 6): Rₜ=0.351 min. MS (Mass method 1): m/z=188 (M+H)⁺

### Intermediate 39

### rac-2-bromo-1-[oxan-3-yl]ethan-1-one

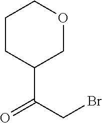

1.00 g (7.68 mmol) of rac-tetrahydro-2H-pyran-3-carboxylic acid were dissolved in 30 mL of dichloromethane. Then, 0.80 mL (9.20 mmol) of oxalyl chloride were added dropwise at room temperature followed by 2 drops of N,N-dimethylformamide (vigorous gas evolution was observed). The reaction mixture was stirred at room temperature for 3.5 hours and then it was concentrated to dryness to afford a yellow oil. The aforementioned residue was dissolved in 15 mL of acetonitrile and 11.00 mL (22.00 mmol) of trimethylsilyldiazomethane (2.0M in hexanes) were added. The reaction mixture was stirred at room temperature for 2 hours and then cooled to 0° C. 0.40 mL (6.40 mmol) of acetic acid and 4.70 mL (28.00 mmol) of hydrobromic acid (33% in acetic acid) were sequentially added dropwise; again, vigorous gas evolution was observed. After the addition was complete, the ice bath was removed and the mixture was warmed to room temperature and stirred for 2 hours. After that time, the reaction was quenched with water and extracted twice with dichloromethane. The organic layers were combined, dried over magnesium sulfate, filtered and concentrated to afford 0.84 g (53%) of the product which was used without further purification.

¹H-NMR (300 MHz, DMSO-d₆): δ [ppm]=1.40-1.67 (m, 3H), 1.90-2.03 (m, 1H), 2.71-2.94 (m, 1H), 3.24-3.44 (m, 2H), 3.74 (d, 1H), 3.94 (d, 1H), 4.49 (s, 1H), 4.65 (s, 1H).

### Intermediate 40

### rac-1-{2-[oxan-3-yl]-2-oxoethyl}-1,3,5,7-tetraazatricyclo[3.3.1.1^{3,7}]decan-1-ium bromide

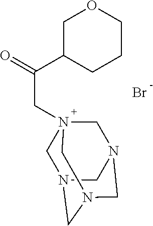

0.84 g (4.06 mmol) of rac-2-bromo-1-[oxan-3-yl]ethan-1-one was dissolved in 12 mL of chloroform and 0.57 g (4.06 mmol) of hexamethylenetetramine was added allowing the mixture to stir at room temperature for 12 hours. The solvent was partially removed and the resulting precipitate was filtered off, washed with cold chloroform and isolated. The remaining mother liquors were concentrated again, filtered and washed with cold chloroform several times until no more precipitate was appreciated. All the collected solids were dried in vacuo to give 1.40 g (99%) of the product as a white powder. The compound was used as such in the next synthetic step.

LC-MS (Method 3): Rₜ=0.187 min. MS (Mass method 1): m/z=267 (M)⁺

### Intermediate 41

### rac-2-amino-1-[oxan-3-yl]ethan-1-one hydrochloride

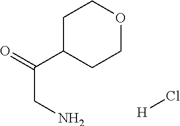

1.40 g (4.03 mmol) of rac-1-{2-[oxan-3-yl]-2-oxoethyl}-1,3,5,7-tetraazatricyclo[3.3.1.1^{3,7}]decan-1-ium bromide was dissolved in 9 mL of ethanol and 3 mL of hydrochloric acid(conc). and the mixture was refluxed for 30 min. The resulting precipitate was filtered off, washed with ethanol and discarded. The filtrates were evaporated and dried to yield 0.72 g (99%) of the product as a thick oil. The compound was used as such in the next step.

LC-MS (Method 3): Rₜ=0.150 min. MS (Mass method 1): m/z=144 (M+H)⁺

### Intermediate 42

### rac-tert-Butyl {2-[oxan-3-yl]-2-oxoethyl}carbamate

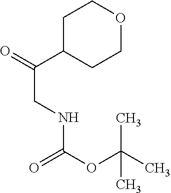 [SMILES: CC(C)(C)OC(=O)NCC(=O)C1CCOCC1]

1.10 mL (8.00 mmol) of triethylamine and 1.10 mL (4.80 mmol) of di-tert-butyl dicarbonate were added to a solution of 0.72 g (4.01 mmol) of rac-2-amino-1-[oxan-3-yl]ethan-1-one hydrochloride in 15 mL of dichloromethane and the mixture was stirred at room temperature for 12 hours. Water was added to the reaction and the phases were separated. The organic layer was dried over magnesium sulfate, filtered and evaporated. The corresponding residue was purified by flash chromatography on silica gel eluting with heptane and ethyl acetate. 0.38 g (40%) of the product were isolated as a pale yellow oil.

¹H-NMR (300 MHz, DMSO-d₆): δ [ppm]=1.38 (s, 9H), 1.48-1.65 (m, 3H), 1.83-1.94 (m, 1H), 2.65-2.79 (m, 1H), 3.29 (d, 1H), 3.37 (d, 1H), 3.70-3.91 (m, 4H), 6.99 (t, 1H).

LC-MS (Method 3): Rₜ=0.590 min. MS (Mass method 1): m/z=266 (M+Na)⁺

### Intermediate 43

### rac-tert-Butyl ({4-[oxan-3-yl]-2,5-dioxoimidazolidin-4-yl}methyl)carbamate

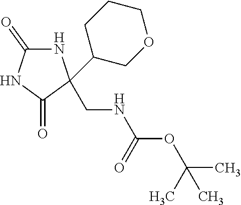 [SMILES: CC(C)(C)OC(=O)NCC1(C2CCCOC2)NC(=O)NC1=O]

To a stirring solution of 2.25 g (23.40 mmol) of ammonium carbonate and 0.33 g (6.25 mmol) of ammonium chloride in 8 mL of water was added 0.38 g (1.56 mmol) of rac-tert-Butyl {2-[oxan-3-yl]-2-oxoethyl}carbamate in 8 mL of ethanol. After 15 min, 0.46 g (7.03 mmol) of potassium cyanide were added and the mixture was heated up to 60° C. for 16 hours into a sealed pressure flask. The yellow solution was concentrated until only a small fraction of water remained (a white precipitate appeared). Then, more water was added and the suspension was allowed to stand at 0° C. for 1 hour. After that time, the resulting solid was filtered off and washed with water and diethyl ether to give 0.18 g (36%) of the product as a white powder. The compound was used as such in the next step.

¹H-NMR (300 MHz, DMSO-d₆): δ [ppm]=1.13-1.31 (m, 1H), 1.37 (s, 9H), 1.43-1.64 (m, 2H), 1.76-1.91 (m, 2H), 3.06-3.27 (m, 4H), 3.61-3.95 (m, 2H), 6.77 (t, 1H), 7.65 (s, 1H), 10.64 (bp, 1H)

LC-MS (Method 3): Rₜ=0.450 min. MS (Mass method 1): m/z=258 (M−tBu+H)⁺

### Intermediate 44

### rac-5-(aminomethyl)-5-[oxan-3-yl]imidazolidine-2,4-dione hydrochloride

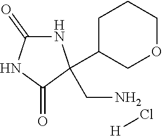

0.72 mL (2.90 mmol) of 4N hydrochloric acid in dioxane were added to a solution of 0.18 g (0.57 mmol) of rac-tert-Butyl ({4-[oxan-3-yl]-2,5-dioxoimidazolidin-4-yl}methyl)carbamate in 5 mL of dichloromethane and the mixture was allowed to stir at room temperature for 16 hours. The resulting precipitate was collected by filtration and washed with dichloromethane, ethyl acetate and diethyl ether to give 0.14 g (97%) of the product as a white solid. The compound was used as such in the next step.

¹H-NMR (300 MHz, DMSO-d₆): δ [ppm]=1.16-1.35 (m, 1H), 1.36-1.67 (m, 3H), 1.76-2.02 (m, 2H), 3.08-3.25 (m, 3H), 3.66-3.90 (m, 2H), 8.02-8.18 (m, 4H), 11.09 (s, 1H).

LC-MS (Method 3): Rₜ=0.121 min. MS (Mass method 1): m/z=214 (M+H)⁺

### Intermediate 45

### 2-bromo-1-(oxan-4-yl)ethan-1-one

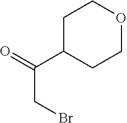

1.20 mL (23.00 mmol) of bromine were added drop wise to a stirred solution of 3.00 g (23.40 mmol) of 1-(oxan-4-yl)ethan-1-one in 15 mL of methanol at −10° C. and the mixture was stirred at 0° C. for 45 min and at 10° C. for 45 min. After this time, an aqueous 11M solution of sulfuric acid (20 ml) was added allowing the mixture to stir at room temperature for 16 hours. Water was added and the reaction was extracted with diethyl ether. The combined organic layers were washed with saturated sodium bicarbonate(aq.) and water. The organic layer was dried over magnesium sulfate, filtered and concentrated to give 3.28 g (68%) of the product as a tan oil. The compound was used as such in the next synthetic step.

¹H-NMR (300 MHz, DMSO-d₆): δ [ppm]=1.49 (qd, 2H), 1.75 (d, 2H), 2.87 (tt, 1H), 3.33 (t, 2H), 3.85 (d, 2H), 4.49 (s, 2H).

LC-MS (Method 3): Rₜ=0.403 min. MS (Mass method 1): m/z=206 (M+H)⁺

### Intermediate 46

### 1-[2-(oxan-4-yl)-2-oxoethyl]-1,3,5,7-tetraazatricyclo[3.3.1.1^{3,7}]decan-1-ium bromide

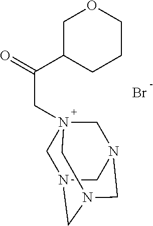

2.09 g (10.10 mmol) of 2-bromo-1-(oxan-4-yl)ethan-1-one was dissolved in 30 mL of chloroform and 1.41 g (10.10 mmol) of hexamethylenetetramine was added allowing the mixture to stir at room temperature for 12 hours. The solvent was partially removed and the resulting precipitate was filtered off, washed with cold chloroform and isolated. The remaining mother liquors were concentrated again, filtered and washed with cold chloroform several times until no more precipitate was appreciated. All the collected solids were dried in vacuo to give 2.16 g (62%) of the product as a white powder. The compound was used as such in the next synthetic step.

¹H-NMR (300 MHz, DMSO-d₆): δ [ppm]=1.49 (qd, 2H), 1.77 (2H), 2.74 (tt, 1H), 3.88 (d, 2H), 4.19 (s, 2H), 4.59 (dd, 6H), 5.29 (s, 6H).

LC-MS (Method 3): Rₜ=0.125 min. MS (Mass method 1): m/z=267 (M)⁺

### Intermediate 47

### 2-amino-1-(oxan-4-yl)ethan-1-one hydrochloride

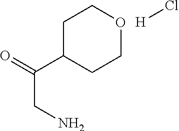

2.16 g (6.22 mmol) of 1-[2-(oxan-4-yl)-2-oxoethyl]-1,3,5,7-tetraazatricyclo[3.3.1.1^{3,7}]decan-1-ium bromide were dissolved in 30 mL of ethanol and 10 mL of hydrochloric acid(conc). and the mixture was refluxed for 30 min. The resulting precipitate was filtered off, washed with ethanol and discarded. The filtrates were evaporated and dried to yield 1.12 g (99%) of the product as a thick oil. The compound was used as such in the next step.

LC-MS (Method 3): Rₜ=0.116 min. MS (Mass method 1): m/z=144 (M+H)⁺

### Intermediate 48

### tert-Butyl [2-(oxan-4-yl)-2-oxoethyl]carbamate

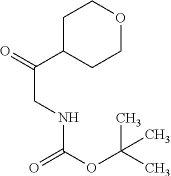 [SMILES: CC(C)(C)OC(=O)NCC(=O)C1CCOCC1]

2.03 mL (16.00 mmol) of triethylamine and 1.80 mL (7.70 mmol) of di-tert-butyl dicarbonate were added to a solution of 1.16 g (6.46 mmol) of 2-amino-1-(oxan-4-yl)ethan-1-one hydrochloride in 20 mL of dichloromethane and the mixture was stirred at room temperature for 12 hours. Water was added to the reaction and the phases were separated. The organic layer was dried over magnesium sulfate, filtered and evaporated. The corresponding residue was purified by flash chromatography on silica gel eluting with heptane and ethyl acetate. 1.05 g (67%) of the product were isolated as a colourless oil.

¹H-NMR (300 MHz, DMSO-d₆): δ [ppm]=1.37 (s, 9H), 1.44 (qd, 2H), 1.66 (d, 2H), 2.68 (tt, 1H), 3.32 (t, 2H), 3.78-3.88 (m, 4H), 6.94 (t, 1H).

LC-MS (Method 3): Rₜ=0.548 min. MS (Mass method 1): m/z=144 (M−Boc+H)⁺

### Intermediate 49

### rac-tert-Butyl {[4-(oxan-4-yl)-2,5-dioxoimidazolidin-4-yl]methyl}carbamate

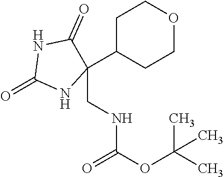 [SMILES: CC(C)(C)OC(=O)NCC1(C2CCOCC2)NC(=O)NC1=O]

To a stirring solution of 6.22 g (64.70 mmol) of ammonium carbonate and 1.15 g (21.60 mmol) of ammonium chloride in 10 mL of water was added 1.05 g (4.32 mmol) of tert-butyl [2-(oxan-4-yl)-2-oxoethyl]carbamate in 10 mL of ethanol. After 15 min, 1.26 g (19.40 mmol) of potassium cyanide were added and the mixture was heated up to 60° C. for 16 hours into a sealed pressure flask. The yellow solution was concentrated until only a small fraction of water remained (a white precipitate appeared). Then, more water was added and the suspension was allowed to stand at 0° C. for 1 hour. After that time, the resulting solid was filtered off and washed with water and diethyl ether to give 0.92 g (68%) of the product as an off-white powder. The compound was used as such in the next step.

¹H-NMR (300 MHz, DMSO-d₆): δ [ppm]=1.19-1.50 (m, 12H), 1.55 (d, 1H), 1.84 (t, 1H), 3.15-3.28 (m, 3H), 3.84 (t, 2H), 6.73 (t, 1H), 7.66 (s, 1H), 10.58 (s, 1H).

LC-MS (Method 3): Rₜ=0.428 min. MS (Mass method 1): m/z=258 (M−tBu+H)⁺

### Intermediate 50

### rac-5-(aminomethyl)-5-(oxan-4-yl)imidazolidine-2,4-dione hydrochloride

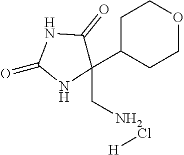

3.70 mL (15.00 mmol) of 4N hydrochloric acid in dioxane were added to a solution of 0.92 g (2.94 mmol) of rac-tert-Butyl {[4-(oxan-4-yl)-2,5-dioxoimidazolidin-4-yl]methyl}carbamate in 20 mL of dichloromethane and the mixture was allowed to stir at room temperature for 16 hours. The resulting precipitate was collected by filtration and washed with dichloromethane, ethyl acetate and diethyl ether to give 0.70 g (95%) of the product as a white solid. The compound was used as such in the next step.

¹H-NMR (300 MHz, DMSO-d₆): δ [ppm]=1.19-1.59 (m, 4H), 1.97 (t, 1H), 3.07 (q, 2H), 3.23 (t, 2H), 3.87 (td, 2H), 8.11 (s, 1H), 8.22 (bp, 3H), 11.03 (bp, 1H).

LC-MS (Method 1): Rₜ=0.331 min. MS (Mass method 1): m/z=214 (M+H)⁺

### Intermediate 51

### rac-2-nitro-1-[oxolan-3-yl]ethan-1-ol

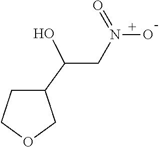

0.57 g (2.50 mmol) of benzyltriethylammonium chloride were added to a solution of 1.40 mL (25.00 mmol) of nitromethane and 4.50 mL of tetrahydrofuran-3-carboxaldehyde (50% in water) in 75 mL of 0.025M sodium hydroxide(aq.) and the mixture was stirred at room temperature for 15 hours. After that time, the reaction was saturated with solid sodium chloride and extracted with diethyl ether. The combined organic phases were dried over magnesium sulfate, filtered and concentrated. The corresponding crude nitro alcohol was purified by f lash chromatography on silica gel eluting with heptane and ethyl acetate to give 1.73 g (43%) of the product as a light yellow oil.

¹H-NMR (300 MHz, DMSO-d₆): δ [ppm]=1.48-164 (m, 0.5H), 1.70-1.95 (m, 1.5H), 2.17-2.35 (m, 1H), 3.44 (t, 0.5H), 3.53-3.65 (m, 1.5H), 3.66-3.79 (m, 2H), 3.95-4.11 (m, 1H), 4.29-4.42 (m, 1H), 4.70 (ddd, 1H), 5.58 (t, 1H).

LC-MS (Method 3): Rₜ=0.149 min. MS (Mass method 1): m/z=162 (M+H)⁺

### Intermediate 52

### rac-2-amino-1-[oxolan-3-yl]ethan-1-ol

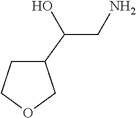

0.09 g (0.83 mmol) of palladium on carbon (10% loading, Degussa type, wet basis) were added to a solution of 1.33 g (8.25 mmol) of rac-2-nitro-1-[oxolan-3-yl]ethan-1-ol in 25 mL of methanol and the resulting suspension was stirred at room temperature under hydrogen atmosphere for 16 hours. The reaction was filtered through a pad of celite and the filtrates were evaporated giving 1.07 g (99%) of the crude product as an oil. The compound was used as such in the next step.

LC-MS (Method 3): Rₜ=0.108 min. MS (Mass method 1): m/z=132 (M+H)⁺

### Intermediate 53

### rac-tert-Butyl {2-hydroxy-2-[oxolan-3-yl]ethyl}carbamate

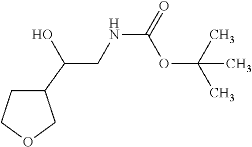 [SMILES: CC(C)(C)OC(=O)NCC(O)C1CCOC1]

1.90 mL (8.50 mmol) of di-tert-butyl dicarbonate were added to a solution of 1.06 g (8.08 mmol) of rac-2-amino-1-[oxolan-3-yl]ethan-1-ol in 25 mL of dichloromethane and the mixture was stirred at room temperature for 2 hours. Volatiles were removed under reduced pressure and the resulting residue was purified by flash chromatography on silica gel eluting with heptane aid ethyl acetate to give 1.36 g (73%) of the product as a thick oil.

¹H-NMR (300 MHz, DMSO-d₆): δ [ppm]=1.49 (s, 9H), 1.45-1.92 (m, 2H), 2.04-2.22 (m, 1H), 2.76-3.12 (m, 2H), 3.47-3.77 (m, 4H), 4.76 (d, 1H), 6.62-6.74 (m, 1H).

LC-MS (Method 3): Rₜ=0.458 min. MS (Mass method 1): m/z=254 (M+Na)⁺

### Intermediate 54

### rac-tert-Butyl {2-oxo-2-[oxolan-3-yl]ethyl}carbamate

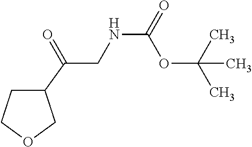 [SMILES: CC(C)(C)OC(=O)NCC(=O)C1CCOC1]

2.16 g (10.00 mmol) of pyridinium chlorochromate were added in portions to a stirred solution of 1.16 g (5.02 mmol) of rac-tert-Butyl {2-hydroxy-2-[oxolan-3-yl]ethyl}carbamate in 15 mL of dichloromethane and the resulting suspension was stirred at room temperature for 15 hours. The reaction was filtered through a pad of celite and the filtrates were washed with water and brine. The organic layer was dried over magnesium sulfate, filtered, concentrated and purified by flash chromatography on silica gel eluting with heptane and ethyl acetate to afford 0.71 g (62%) of the product as a colourless oil.

¹H-NMR (300 MHz, DMSO-d₆): δ [ppm]=1.38 (s, 9H), 1.87-2.08 (m, 2H), 3.23-3.38 (m, 3H), 3.57-3.86 (m, 6H), 7.08 (t, 1H).

LC-MS (Method 3): Rₜ=0.508 min. MS (Mass method 1): m/z=252 (M+Na)⁺

### Intermediate 55

### rac-tert-Butyl ({2,5-dioxo-4-[oxolan-3-yl]imidazolidin-4-yl}methyl)carbamate

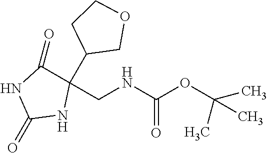 [SMILES: CC(C)(C)OC(=O)NCC1(C2CCOC2)NC(=O)NC1=O]

To a stirring solution of 4.46 g (46.50 mmol) of ammonium carbonate and 0.66 g (12.40 mmol) of ammonium chloride in 5 mL of water was added 0.71 g (3.10 mmol) of rac-tert-Butyl {2-oxo-2-[oxolan-3-yl]ethyl}carbamate in 15 mL of ethanol. After 15 min, 0.91 g (13.90 mmol) of potassium cyanide were added and the mixture was heated up to 60° C. for 16 hours into a sealed pressure flask. The yellow solution was concentrated until only a small fraction of water remained (a white precipitate appeared). Then, more water was added and the suspension was allowed to stand at 0° C. for 1 hour. After that time, the resulting solid was filtered off and washed with water and diethyl ether to give 0.64 g (69%) of the product as an off-white powder. The compound was used as such in the next step.

¹H-NMR (300 MHz, DMSO-d₆): δ [ppm]=1.37 (s, 9H), 1.46-2.03 (m, 2H), 3.06-3.29 (m, 3H), 3.38-3.84 (m, 4H), 6.78-6.95 (m, 1H), 7.77 (d, 1H), 10.67 (bp, 1H).

LC-MS (Method 3): Rₜ=0.347 min. MS (Mass method 1): m/z=322 (M+Na)⁺

### Intermediate 56

### rac-5-(aminomethyl)-5-[oxolan-3-yl]imidazolidine-2,4-dione hydrochloride

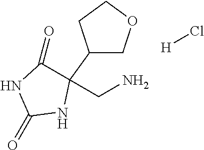

2.60 mL (11.00 mmol) of 4N hydrochloric acid in dioxane were added to a solution of 0.63 g (2.10 mmol) of rac-tert-Butyl ({2,5-dioxo-4-[oxolan-3-yl]imidazolidin-4-yl}methyl)carbamate in 10 mL of dichloromethane and the mixture was allowed to stir at room temperature for 16 hours. The resulting precipitate was collected by filtration and washed with dichloromethane, ethyl acetate and diethyl ether to give 0.44 g (89%) of the product as a white solid. The compound was used as such in the next step.

¹H-NMR (300 MHz, DMSO-d₆): δ [ppm]=1.51-2.05 (m, 2H), 2.54-2.69 (m, 1H), 2.90-3.06 (m, 1H), 3.09-3.22 (m, 1H), 3.43-3.84 (m, 4H), 8.15-3.38 (m, 4H), 11.11 (s, 1H).

LC-MS (Method 1): Rₜ=0.313 min. MS (Mass method 1): m/z=200 (M+H)⁺

### Intermediate 57

### rac-1-[2,2-dimethylcyclopropyl]-2-nitroethan-1-one

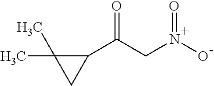

5.00 g (43.80 mmol) of 2,2-dimethylcyclopropanecarboxylic acid and 8.52 g (52.60 mmol) of 1,1′-carbonyldiimidazole where dissolved in 70 mL of N,N-dimethylformamide under nitrogen atmosphere at room temperature and the mixture was stirred for 2 hours. In a separate flask, 3.60 mL (66.00 mmol) of nitromethane were slowly added to a suspension of 2.10 g (52.60 mmol) of sodium hydride in 70 mL of N,N-dimethylformamide under nitrogen atmosphere at room temperature and the mixture was stirred for 2 hours. After that time, this suspension was cooled to 0° C. and the solution containing the activated carboxylic acid was added slowly over 5 min keeping internal temperature below 10° C. Once the addition finished, the mixture was stirred at 0° C. for 2 hours and at room temperature for 12 additional hours. The reaction mixture was cooled at 0° C. and quenched with 2N hydrochloric acid(aq.) and water. The resulting mixture was extracted with dichloromethane and the combined organic extracts were dried over magnesium sulfate, filtered and concentrated to afford a yellow residue, which was purified by flash column chromatography on silica gel eluting with heptane/ethyl acetate mixtures to give 4.93 g (72%) of the product as a yellowish oil.

¹H-NMR (300 MHz, DMSO-d₆): δ [ppm]=1.06 (s, 4H), 1.21 (s, 4H), 2.02 (t, 1H), 5.92 (d, 2H).

LC-MS (Method 3): Rₜ=0.631 min. MS (Mass method 1): m/z=158 (M+H)⁺

### Intermediate 58

### rac-tert-Butyl {2-[2,2-dimethylcyclopropyl]-2-oxoethyl}carbamate

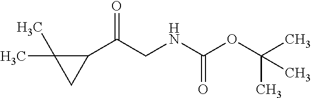 [SMILES: CC(C)(C)OC(=O)NCC(=O)C1CC1(C)C]

0.03 g (0.29 mmol) of palladium on carbon (10% loading, Degussa type, wet basis) were added to a solution of 4.57 g (29.10 mmol) of rac-1-[2,2-dimethylcyclopropyl]-2-nitroethan-1-one and 7.00 mL (31.00 mmol) of di-tert-butyl dicarbonate in 10 mL of methanol and the mixture was stirred at room temperature under hydrogen atmosphere for 16 hours. The solution was then filtered through a pad of celite, and the filtrates were concentrated to dryness to give a residue which was purified by flash chromatography on silica gel eluting with heptane/ethyl acetate mixtures to afford 3.80 g (57%) of the product as a colourless oil.

¹H-NMR (300 MHz, DMSO-d₆): δ [ppm]=0.82-0.90 (m, 1H), 0.93-1.01 (m, 4H), 1.02-1.09 (m, 1H), 1.15 (s, 3H), 1.38 (s, 9H), 1.97 (t, 1H), 3.76 (ddd, 2H), 7.09 (t, 1H).

LC-MS (Method 3): Rₜ=0.813 min. MS (Mass method 1): m/z=250 (M+Na)⁺

### Intermediate 59

### rac-tert-Butyl ({4-[2,2-dimethylcyclopropyl]-2,5-dioxoimidazolidin-4-yl}methyl)carbamate

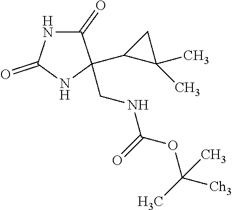

To a stirring solution of 6.34 g (66.00 mmol) of ammonium carbonate and 0.94 g (17.60 mmol) of ammonium chloride in 10 mL of water was added 1.00 g (4.40 mmol) of rac-tert-Butyl {2-[2,2-dimethylcyclopropyl]-2-oxoethyl}carbamate in 10 mL of ethanol. After 15 min, 1.29 g (19.80 mmol) of potassium cyanide were added and the mixture was heated up to 60° C. for 16 hours into a sealed pressure flask. The yellow solution was concentrated until only a small fraction of water remained (a white precipitate appeared). Then, more water was added and the suspension was allowed to stand at 0° C. for 1 hour. After that time, the resulting solid was filtered off and washed with water and diethyl ether to give 0.80 g (61%) of the product as an off-white powder. The compound was used as such in the next step.

¹H-NMR (300 MHz, DMSO-d₆): δ [ppm]=0.32-0.48 (m, 2H), 0.79-0.85 (m, 1H), 0.87 (s, 3H), 1.00 (s, 3H), 1.06 (s, 2H), 1.36 (s, 9H), 3.26-3.31 (m, 2H), 6.73 (t, 1H), 7.29 (s, 1H), 10.64 (bp, 1H)

LC-MS (Method 3): Rₜ=0.646 min. MS (Mass method 1): m/z=242 (M−tBu+H)⁺

### Intermediate 60

### rac-5-(aminomethyl)-5-[2,2-dimethylcyclopropyl]imidazolidine-2,4-dione hydrochloride

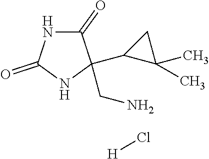

1.60 mL (6.20 mmol) of 4N hydrochloric acid in dioxane were added to a solution of 0.37 g (2.10 mmol) of rac-tert-Butyl ({4-[2,2-dimethylcyclopropyl]-2,5-dioxoimidazolidin-4-yl}methyl)carbamate in 10 mL of dichloromethane and the mixture was allowed to stir at room temperature for 16 hours. The resulting precipitate was collected by filtration and washed with dichloromethane, ethyl acetate and diethyl ether to give 0.33 g (98%) of the product as a white solid. The compound was used as such in the next step.

¹H-NMR (300 MHz, DMSO-d₆): δ [ppm]=0.44-0.59 (m, 2H), 1.02 (s, 3H), 1.08 (s, 3H), 2.94-3.09 (m, 1H), 3.13-3.26 (m, 1H), 7.98 (s, 1H), 8.27 (bp, 3H), 11.01 (s, 1H).

LC-MS (Method 3): Rₜ=0.144 min. MS (Mass method 1): m/z=198 (M+H)⁺

### Intermediate 61

### rac-2-Nitro-1-(pyridin-3-yl)ethan-1-ol

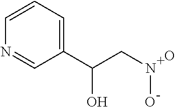

0.40 g (2.33 mmol) of barium hydroxide were added to a solution of 5.00 g (46.70 mmol) of nicotinaldehyde and 25.00 mL (470.00 mmol) of nitromethane in 135 mL of water and the mixture was stirred at room temperature for 15 min. The reaction was then extracted three times with ethyl acetate. The combined organic layers were dried over magnesium sulfate, filtered, and concentrated in vacuo to give 7.40 g (94%) of the crude product as a brown oil. The compound was used as such in the next step.

¹H-NMR (300 MHz, DMSO-d₆): δ [ppm]=4.68 (dd, 1H), 4.92 (dd, 1H), 5.29-5.40 (m, 1H), 6.24 (d, 1H), 7.40 (dd, 1H), 7.85 (d, 1H), 8.51 (d, 1H), 8.64 (s, 1H).

LC-MS (Method 3): Rₜ=0.115 min. MS (Mass method 1): m/z=169 (M+H)⁺

### Intermediate 62

### rac-3-[1-{[tert-butyl(dimethyl)silyl]oxy}-2-nitroethyl]pyridine

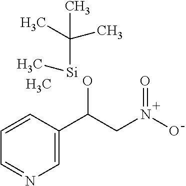

6.27 g (41.60 mmol) of tert-butyldimethylsilyl chloride were added to a solution of 2.00 (11.90 mmol) of rac-2-nitro-1-(pyridin-3-yl)ethan-1-ol and 4.05 g (59.50 mmol) of imidazole in 20 mL of N,N-dimethylformamide at 0° C. and the mixture was allowed to stir at room temperature for 14 hours. The solution was diluted with ethyl acetate and washed with water and brine. The organic layer was dried over magnesium sulfate, filtered and evaporated to dryness to yield a residue, which was purified by flash chromatography on silica gel eluting with heptane/ethyl acetate mixtures to afford 0.80 g (24%) of the product as a colourless oil.

LC-MS (Method 3): Rₜ=0.929 min. MS (Mass method 1): m/z=283 (M+H)⁺

### Intermediate 63

### rac-2-{[tert-butyl(dimethyl)silyl]oxy}-2-(pyridin-3-yl)ethan-1-amine

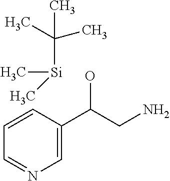

A mixture of 680 mg (2.41 mmol) of rac-3-[1-{[tert-butyl(dimethyl)silyl]oxy}-2-nitroethyl]pyridine and 3 mg (0.02 mmol) of palladium on carbon (10% loading, Degussa type, wet basis) in 10 mL of methanol was stirred under hydrogen atmosphere at room temperature for 12 hours. After that time, the catalyst was removed by filtration through a pad of celite and the cake was thoroughly washed with methanol. Evaporation of the solvent furnished a residue which was purified by flash chromatography on silica gel eluting with dichloromethane/methanol mixtures to afford 573 mg (94%) of the product as a colourless oil.

LC-MS (Method 3): Rₜ=0.562 min. MS (Mass method 1): m/z=253 (M+H)⁺

### Intermediate 64

### rac-tert-Butyl [2-{[tert-butyl(dimethyl)silyl]oxy}-2-(pyridin-3-yl)ethyl]carbamate

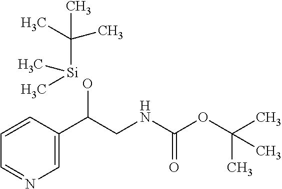

0.55 mL (2.40 mmol) of di-tert-butyl dicarbonate were added to a solution of 573 mg (2.27 mmol) of rac-2-{[tert-butyl(dimethyl)silyl]oxy}-2-(pyridin-3-yl)ethanamine in 20 mL of dichloromethane and the mixture was stirred at room temperature for 12 hours. The reaction was washed with water and brine and the organic layer was dried over magnesium sulfate, filtered and evaporated to dryness to afford 800 mg (99%) of the crude product as a yellow oil. The compound was used as such in the next synthetic step.

LC-MS (Method 3): Rₜ=0.984 min. MS (Mass method 1): m/z=353 (M+H)⁺

### Intermediate 65

### rac-tert-Butyl [2-hydroxy-2-(pyridin-3-yl)ethyl]carbamate

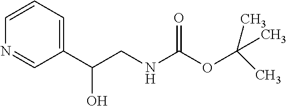 [SMILES: CC(C)(C)OC(=O)NCC(O)c1cccnc1]

2.11 mL (2.11 mmol) of 1N tetra-n-butylammonium fluoride in tetrahydrofuran was added dropwise to a stirred solution of 495 mg (1.40 mmol) of rac-tert-Butyl [2-{[tert-butyl(dimethyl)silyl]oxy}-2-(pyridin-3-yl)ethyl]carbamate in 10 mL of anhydrous tetrahydrofuran under nitrogen atmosphere and the mixture was stirred at room temperature for 14 hours. After that time, the solution was poured onto 50 mL of water, and extracted with ethyl acetate. The combined organic extracts were washed successively with water and brine, dried over magnesium sulfate, and concentrated in vacuo to furnish 334 mg (99%) of the crude product as a brown powder. The compound was used as such in the next step.

LC-MS (Method 3): Rₜ=0.249 min. MS (Mass method 1): m/z=239 (M+H)⁺

### Intermediate 66

### tert-Butyl [2-oxo-2-(pyridin-3-yl)ethyl]carbamate

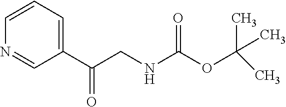 [SMILES: CC(C)(C)OC(=O)NCC(=O)c1cccnc1]

0.49 mL (6.90 mmol) of dimethyl sulfoxide were added drop wise to a solution of 0.30 mL (3.40 mmol) of oxalyl chloride in 8 mL of anhydrous dichloromethane at −78° C. and the mixture was stirred under nitrogen atmosphere under these conditions for 30 min. 409 mg (1.72 mmol) of rac-tert-Butyl [2-hydroxy-2-(pyridin-3-yl)ethyl]carbamate was added to the cold solution and the resulting mixture was stirred for 15 additional min. After that, 1.20 mL (8.60 mmol) of triethylamine were added and the reaction was allowed to stir at 0° C. for 30 min. Finally, the mixture was diluted with ethyl acetate and the organic layer was washed once with water and brine, dried over magnesium sulfate, filtered and concentrated in vacuo. Flash chromatography of the resulting residue using heptane/ethyl acetate mixtures as eluent gave 190 mg (47%) of the product as a white solid.

LC-MS (Method 3): Rₜ=0.535 min. MS (Mass method 1): m/z=237 (M+H)⁺

### Intermediate 67

### rac-tert-Butyl {[2,5-dioxo-4-(pyridin-3-yl)imidazolidin-4-yl]methyl}carbamate

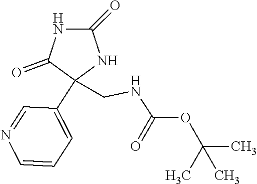 [SMILES: CC(C)(C)OC(=O)NCC1(c2cccnc2)NC(=O)NC1=O]

To a stirring solution of 1.10 g (11.40 mmol) of ammonium carbonate and 0.16 g (3.05 mmol) of ammonium chloride in 2 mL of water was added 0.18 g (0.76 mmol) of tert-butyl [2-oxo-2-(pyridin-3-yl)ethyl]carbamate in 2 mL of ethanol. After 15 min, 0.22 g (3.43 mmol) of potassium cyanide were added and the mixture was heated up to 60° C. for 16 hours into a sealed pressure flask. The yellow solution was concentrated until only a small fraction of water remained (a white precipitate appeared). Then, more water was added and the suspension was allowed to stand at 0° C. for 2 hour. After that time, the resulting solid was filtered off and washed with cold water and diethyl ether to give 0.14 g (60%) of the product as an off-white powder. The compound was used as such in the next step.

¹H-NMR (300 MHz, DMSO-d₆): δ [ppm]=1.29 (s, 9H), 3.39-3.64 (m, 2H), 6.88 (bp, 1H), 7.38 (dd, 1H), 7.89 (d, 1H), 8.50 (d, 1H), 8.69 (s, 1H).

LC-MS (Method 3): Rₜ=0.348 min. MS (Mass method 1): m/z=307 (M+H)⁺

### Intermediate 68

### rac-5-(aminomethyl)-5-(pyridin-3-yl)imidazolidine-2,4-dione hydrochloride

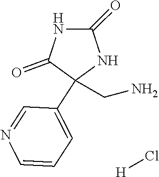 [SMILES: NCC1(c2cccnc2)NC(=O)NC1=O ; Cl]

0.78 mL (3.10 mmol) of 4N hydrochloric acid in dioxane were added to a solution of 0.19 g (0.62 mmol) of rac-tert-Butyl{[2,5-dioxo-4-(pyridin-3-yl)imidazolidin-4-yl]methyl}carbamate in 10 mL of dichloromethane and the mixture was allowed to stir at room temperature for 16 hours. The resulting precipitate was collected by filtration and washed with dichloromethane, ethyl acetate and diethyl ether to give 0.15 g (99%) of the product as a white solid. The compound was used as such in the next step.

¹H-NMR (300 MHz, DMSO-d₆): δ [ppm]=3.52-3.77 (m, 2H), 7.98 (dd, 1H), 8.53-8.73 (m, 4H), 8.88 (d, 1H), 9.00 (s, 1H), 9.34 (s, 1H), 11.41 (s, 1H).

LC-MS (Method 3): Rₜ=0.126 min. MS (Mass method 1): m/z=207 (M+H)⁺

### Intermediate 69

### rac-2-nitro-1-(pyridin-2-yl)ethan-1-ol

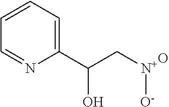

0.40 g (2.33 mmol) of barium hydroxide were added to a solution of 5.00 g (46.70 mmol) of pyridine-2-carbaldehyde and 25.00 mL (470.00 mmol) of nitromethane in 140 mL of water and the mixture was stirred at room temperature for 15 min. The reaction was then extracted three times with ethyl acetate. The combined organic layers were dried over magnesium sulfate, filtered, and concentrated in vacuo to give 7.70 g (98%) of the crude product as a colourless oil. The compound was used as such in the next step.

LC-MS (Method 3): Rₜ=0.163 min. MS (Mass method 1): m/z=169 (M+H)⁺

### Intermediate 70

### rac-2-[1-{[tert-butyl(dimethyl)silyl]oxy}-2-nitroethyl]pyridine

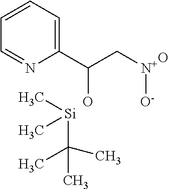

24.20 g (160.00 mmol) of tert-butyldimethylsilyl chloride were added to a solution of 7.70 (45.80 mmol) of rac-2-nitro-1-(pyridin-2-yl)ethan-1-ol and 15.60 g (229.00 mmol) of imidazole in 240 mL of N,N-dimethylformamide at 0° C. and the mixture was allowed to stir at room temperature for 14 hours. The solution was diluted with ethyl acetate and washed with water and brine. The organic layer was dried over magnesium sulfate, filtered and evaporated to dryness to yield a residue, which was purified by flash chromatography on silica gel eluting with heptane/ethyl acetate mixtures to afford 11.85 g (92%) of the product as a colourless oil.

¹H-NMR (300 MHz, DMSO-d₆): δ [ppm]=−0.10 (s, 3H), 0.01 (s, 3H), 0.82 (s, 9H), 4.75 (dd, 1H), 4.95 (dd, 1H), 5.43 (dd, 1H), 7.36 (dd, 1H), 7.54 (d, 1H), 7.88 (t, 1H), 8.56 (d, 1H).

LC-MS (Method 3): Rₜ=1.078 min. MS (Mass method 1): m/z=283 (M+H)⁺

### Intermediate 71

### rac-2-{[tert-butyl(dimethyl)silyl]oxy}-2-(pyridin-2-yl)ethan-1-amine

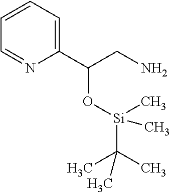 [SMILES: CC(C)(C)[Si](C)(C)OC(CN)c1ccccn1]

A mixture of 11.80 g (41.90 mmol) of rac-2-[1-{[tert-butyl(dimethyl)silyl]oxy}-2-nitroethyl]pyridine and 0.04 g (0.42 mmol) of palladium on carbon (10% loading, Degussa type, wet basis) in 50 mL of methanol was stirred under hydrogen atmosphere at room temperature for 12 hours. After that time, the catalyst was removed by filtration through a pad of celite and the cake was thoroughly washed with methanol. Evaporation of the solvent furnished a residue which was purified by flash chromatography on silica gel eluting with dichloromethane/methanol mixtures to afford 4.52 g (43%) of the product as a colourless oil.

¹H-NMR (300 MHz, DMSO-d₆): δ [ppm]=−0.08 (s, 3H), 0.06 (s, 3H), 0.87 (s, 9H), 2.74 (ddd, 2H), 4.66 (dd, 1H), 7.25 (dd, 1H), 7.41 (d, 1H), 7.78 (t, 1H), 8.48 (d, 1H).

LC-MS (Method 3): Rₜ=0.642 min. MS (Mass method 1): m/z=253 (M+H)⁺

### Intermediate 72

### rac-tert-Butyl [2-{[tert-butyl(dimethyl)silyl]oxy}-2-(pyridin-2-yl)ethyl]carbamate

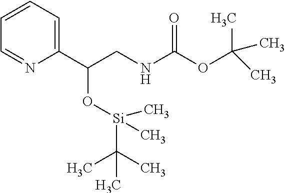 [SMILES: CC(C)(C)OC(=O)NCC(O[Si](C)(C)C(C)(C)C)c1ccccn1]

4.30 mL (19.00 mmol) of di-tert-butyl dicarbonate were added to a solution of 4.51 g (17.90 mmol) of rac-2-{[tert-butyl(dimethyl)silyl]oxy}-2-(pyridin-2-yl)ethanamine in 20 mL of dichloromethane and the mixture was stirred at room temperature for 12 hours. The reaction was washed with water and brine and the organic layer was dried over magnesium sulfate, filtered and evaporated to dryness to afford 6.31 g (99%) of the crude product as a light yellow oil. The compound was used as such in the next synthetic step.

LC-MS (Method 3): Rₜ=1.084 min. MS (Mass method 1): m/z=353 (M+H)⁺

### Intermediate 73

### rac-tert-Butyl [2-hydroxy-2-(pyridin-2-yl)ethyl]carbamate

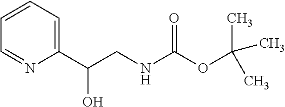 [SMILES: CC(C)(C)OC(=O)NCC(O)c1ccccn1]

27.00 mL (27.00 mmol) of 1N tetra-n-butylammonium fluoride in tetrahydrofuran was added dropwise to a stirred solution of 6.31 g (17.90 mmol) of rac-tert-Butyl [2-{[tert-butyl(dimethyl)silyl]oxy}-2-(pyridin-2-yl)ethyl]carbamate in 100 mL of tetrahydrofuran and the mixture was stirred at room temperature for 14 hours. After that time, the solution was poured onto 50 mL of water, and extracted with ethyl acetate. The combined organic extracts were washed successively with water and brine, dried over magnesium sulfate, and concentrated in vacuo to furnish 4.26 g (99%) of the crude product as a colourless thick oil. The compound was used as such in the next step.

LC-MS (Method 3): Rₜ=0.201 min. MS (Mass method 1): m/z=239 (M+H)⁺

### Intermediate 74

### tert-Butyl [2-oxo-2-(pyridin-2-yl)ethyl]carbamate

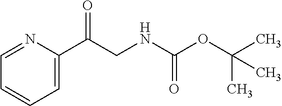 [SMILES: CC(C)(C)OC(=O)NCC(=O)c1ccccn1]

1.19 mL (16.90 mmol) of dimethyl sulfoxide were added drop wise to a solution of 0.73 mL (8.39 mmol) of oxalyl chloride in 20 mL of anhydrous dichloromethane at −78° C. and the mixture was stirred under nitrogen atmosphere under these conditions for 30 min. 1.00 g (4.20 mmol) of rac-tert-Butyl [2-hydroxy-2-(pyridin-2-yl)ethyl]carbamate was added to the cold solution and the resulting mixture was stirred for 15 additional min. After that, 2.92 mL (20.98 mmol) of triethylamine were added and the reaction was allowed to stir at 0° C. for 30 min. Finally, the mixture was diluted with ethyl acetate and the organic layer was washed once with water and brine, dried over magnesium sulfate, filtered and concentrated in vacuo. Flash chromatography of the resulting residue using heptane/ethyl acetate mixtures as eluent gave 0.41 g (41%) of the product as a light brown solid.

¹H-NMR (300 MHz, DMSO-d₆): δ [ppm]=1.39 (s, 9H), 4.58 (d, 2H), 7.00 (t, 1H), 7.70 (dd, 1H), 7.93-8.08 (m, 2H), 8.73 (d, 1H).

LC-MS (Method 3): Rₜ=0.661 min. MS (Mass method 1): m/z=181 (M−tBu+H)⁺

### Intermediate 75

### rac-tert-Butyl {[2,5-dioxo-4-(pyridin-2-yl)imidazolidin-4-yl]methyl}carbamate

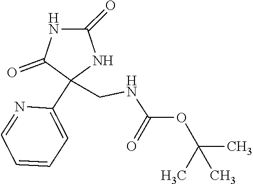 [SMILES: CC(C)(C)OC(=O)NCC1(c2ccccn2)NC(=O)NC1=O]

To a stirring solution of 3.29 g (34.30 mmol) of ammonium carbonate and 0.49 g (9.14 mmol) of ammonium chloride in 6 mL of water was added 0.54 g (2.29 mmol) of tert-butyl [2-oxo-2-(pyridin-2-yl)ethyl]carbamate in 6 mL of ethanol. After 15 min, 0.67 g (10.30 mmol) of potassium cyanide were added and the mixture was heated up to 60° C. for 16 hours into a sealed pressure flask. The yellow solution was concentrated until only a small fraction of water remained (a white precipitate appeared). Then, more water was added and the suspension was allowed to stand at 0° C. for 2 hour. After that time, the resulting solid was filtered off and washed with cold water and diethyl ether to give 0.70 g (98%) of the product as an off-white powder. The compound was used as such in the next step.

LC-MS (Method 3): Rₜ=0.466 min. MS (Mass method 1): m/z=307 (M+H)⁺

### Intermediate 76

### rac-5-(aminomethyl)-5-(pyridin-2-yl)imidazolidine-2,4-dione hydrochloride

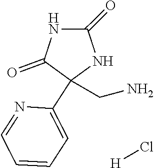 [SMILES: NCC1(c2ccccn2)NC(=O)NC1=O ; Cl]

2.90 mL (11.00 mmol) of 4N hydrochloric acid in dioxane were added to a solution of 0.70 g (2.29 mmol) of rac-tert-Butyl {[2,5-dioxo-4-(pyridin-2-yl)imidazolidin-4-yl]methyl}carbamate in 10 mL of dichloromethane and the mixture was allowed to stir at room temperature for 16 hours. The resulting precipitate was collected by filtration and washed with dichloromethane, ethyl acetate and diethyl ether to give 0.55 g (99%) of the product as a white solid. The compound was used as such in the next step.

LC-MS (Method 3): Rₜ=0.091 min. MS (Mass method 1): m/z=207 (M+H)⁺

### Intermediate 77

### rac-2-nitro-1-(pyridin-4-yl)ethan-1-ol

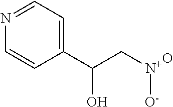

0.40 g (2.33 mmol) of barium hydroxide were added to a solution of 5.00 g (46.70 mmol) of pyridine-4-carbaldehyde and 25.00 mL (470.00 mmol) of nitromethane in 140 mL of water and the mixture was stirred at room temperature for 15 min. The reaction was then extracted three times with ethyl acetate. The combined organic layers were dried over magnesium sulfate, filtered, and concentrated in vacuo to give 7.00 g (89%) of the crude product as a colourless oil. The compound was used as such in the next step.

¹H-NMR (300 MHz, DMSO-d₆): δ [ppm]=4.60 (dd, 1H), 4.95 (dd, 2H), 5.25-5.35 (m, 1H), 6.32 (d, 1H), 7.46 (d, 2H), 8.56 (d, 2H).

LC-MS (Method 3): Rₜ=0.093 min. MS (Mass method 1): m/z=169 (M+H)⁺

### Intermediate 78

### rac-4-[1-{[tert-butyl(dimethyl)silyl]oxy}-2-nitroethyl]pyridine

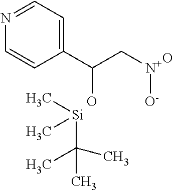

6.71 g (44.50 mmol) of tert-butyldimethylsilyl chloride were added to a solution of 3.75 (22.30 mmol) of rac-2-nitro-1-(pyridin-4-yl)ethan-1-ol and 6.06 g (89.10 mmol) of imidazole in 20 mL of N,N-dimethylformamide at 0° C. and the mixture was allowed to stir at room temperature for 14 hours. The solution was diluted with ethyl acetate and washed with water and brine. The organic layer was dried over magnesium sulfate, filtered and evaporated to dryness to yield a residue, which was purified by flash chromatography on silica gel eluting with heptane/ethyl acetate mixtures to afford 6.29 g (99%) of the product as a colourless oil.

¹H-NMR (300 MHz, DMSO-d₆): δ [ppm]=−0.13 (s, 3H), 0.01 (s, 3H), 0.81 (s, 9H), 4.66 (dd, 1H), 4.89 (dd, 1H), 5.48 (dd, 1H), 7.47 (d, 2H), 8.59 (d, 2H).

LC-MS (Method 3): Rₜ=0.845 min. MS (Mass method 1): m/z=283 (M+H)⁺

### Intermediate 79

### rac-2-{[tert-butyl(dimethyl)silyl]oxy}-2-(pyridin-4-yl)ethan-1-amine

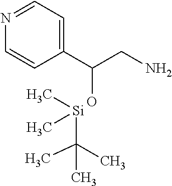

A mixture of 4.51 g (16.00 mmol) of rac-4-[1-{[tert-butyl(dimethyl)silyl]oxy}-2-nitroethyl]pyridine and 0.02 g (0.16 mmol) of palladium on carbon (10% loading, Degussa type, wet basis) in 50 mL of methanol was stirred under hydrogen atmosphere at room temperature for 12 hours. After that time, the catalyst was removed by filtration through a pad of celite and the cake was thoroughly washed with methanol. Evaporation of the solvent furnished a residue which was purified by flash chromatography on silica gel eluting with dichloromethane/methanol mixtures to afford 3.97 g (98%) of the product as a colourless oil.

LC-MS (Method 3): Rₜ=0.357 min. MS (Mass method 1): m/z=253 (M+H)⁺

### Intermediate 80

### rac-tert-Butyl [2-{[tert-butyl(dimethyl)silyl]oxy}-2-(pyridin-4-yl)ethyl]carbamate

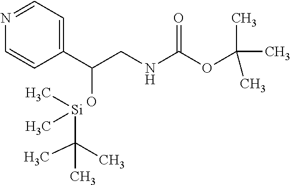

3.80 mL (17.00 mmol) of di-tert-butyl dicarbonate were added to a solution of 3.97 g (15.70 mmol) of rac-2-{[tert-butyl(dimethyl)silyl]oxy}-2-(pyridin-4-yl)ethanamine in 30 mL of dichloromethane and the mixture was stirred at room temperature for 12 hours. The reaction was washed with water and brine and the organic layer was dried over magnesium sulfate, filtered and evaporated to dryness to afford 5.54 g (99%) of the crude product as a light yellow oil. The compound was used as such in the next synthetic step.

LC-MS (Method 3): Rₜ=0.829 min. MS (Mass method 1): m/z=353 (M+H)⁺

### Intermediate 81

### rac-tert-Butyl [2-hydroxy-2-(pyridin-4-yl)ethyl]carbamate

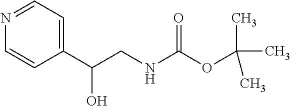 [SMILES: CC(C)(C)OC(=O)NCC(O)c1ccncc1]

23.00 mL (23.00 mmol) of 1N tetra-n-butylammonium fluoride in tetrahydrofuran was added dropwise to a stirred solution of 5.50 g (15.60 mmol) of rac-tert-Butyl [2-{[tert-butyl(dimethyl)silyl]oxy}-2-(pyridin-4-yl)ethyl]carbamate in 50 mL of tetrahydrofuran and the mixture was stirred at room temperature for 14 hours. After that time, the solution was poured onto 50 mL of water, and extracted with ethyl acetate. The combined organic extracts were washed successively with water and brine, dried over magnesium sulfate, and concentrated in vacuo to furnish 3.72 g (99%) of the crude product as a colourless thick oil. The compound was used as such in the next step.

LC-MS (Method 3): Rₜ=0.172 min. MS (Mass method 1): m/z=239 (M+H)⁺

### Intermediate 82

### tert-Butyl [2-oxo-2-(pyridin-4-yl)ethyl]carbamate

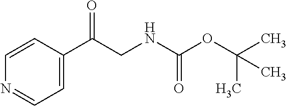 [SMILES: CC(C)(C)OC(=O)NCC(=O)c1ccncc1]

5.34 g (12.59 mmol) of Dess-Martin periodinane were added to a solution of 2.00 (8.39 mmol) of rac-tert-Butyl [2-hydroxy-2-(pyridin-4-yl)ethyl]carbamate in 40 mL of dichloromethane and the mixture was stirred at room temperature for 5 min. Then, a solution of 0.22 mL (12.59 mmol) of water in dichloromethane (1 uL water/1 mL dichloromethane) was added and the mixture was stirred at room temperature for 10 min. After that time, the solution was diluted with dichloromethane and washed with saturated sodium bisulfite(aq.) and saturated sodium bicarbonate(aq.). The organic layer was dried over magnesium sulfate, filtered and evaporated to dryness. The residue was purified by flash chromatography on silica gel eluting with heptane/ethyl acetate mixtures to provide 0.82 g (41%) of the product as a yellowish oil.

LC-MS (Method 3): Rₜ=0.528 min. MS (Mass method 1): m/z=237 (M+H)⁺

### Intermediate 83

### rac-tert-Butyl {[2,5-dioxo-4-(pyridin-4-yl)imidazolidin-4-yl]methyl}carbamate

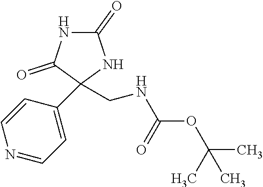 [SMILES: CC(C)(C)OC(=O)NCC1(c2ccncc2)NC(=O)NC1=O]

To a stirring solution of 1.55 g (16.10 mmol) of ammonium carbonate and 0.23 g (4.30 mmol) of ammonium chloride in 2 mL of water was added 0.25 g (1.08 mmol) of tert-butyl [2-oxo-2-(pyridin-4-yl)ethyl]carbamate in 2 mL of ethanol. After 15 min, 0.31 g (4.84 mmol) of potassium cyanide were added and the mixture was heated up to 60° C. for 16 hours into a sealed pressure flask. The yellow solution was concentrated until only a small fraction of water remained (a white precipitate appeared). Then, more water was added and the suspension was allowed to stand at 0° C. for 2 hour. After that time, the resulting solid was filtered off and washed with cold water and diethyl ether to give 0.33 g (98%) of the product as a brown oil. The compound was used as such in the next step.

LC-MS (Method 3): Rₜ=0.289 min. MS (Mass method 1): m/z=307 (M+H)⁺

### Intermediate 84

### rac-5-(aminomethyl)-5-(pyridin-4-yl)imidazolidine-2,4-dione hydrochloride

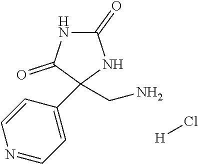 [SMILES: NCC1(c2ccncc2)NC(=O)NC1=O ; Cl]

0.41 mL (1.63 mmol) of 4N hydrochloric acid in dioxane were added to a solution of 0.10 g (0.32 mmol) of rac-tert-Butyl{[2,5-dioxo-4-(pyridin-4-yl)imidazolidin-4-yl]methyl}carbamate in 10 mL of dichloromethane and the mixture was allowed to stir at room temperature for 16 hours. The resulting precipitate was collected by filtration and washed with dichloromethane, ethyl acetate and diethyl ether to give 0.08 g (99%) of the product as a white solid. The compound was used as such in the next step.

LC-MS (Method 3): Rₜ=0.090 min. MS (Mass method 1): m/z=207 (M+H)⁺

### Intermediate 85

### 1-(2-Cyclopropyl-2-oxoethyl)-3,5,7-triaza-1-azoniatricyclo[3.3.1.1^{3,7}]decane bromide

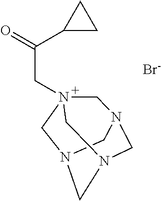

100.00 g (0.61 mol) of 2-bromo-1-cyclopropylethanone was dissolved in 1.4 L of chloroform aid 86.00 g (0.61 mol) of hexamethylenetetramine was added allowing the mixture to stir at room temperature for 12 hours. The solvent was partially removed and the resulting precipitate was filtered off, washed with cold chloroform and isolated. The remaining mother liquors were concentrated again, filtered and washed with cold chloroform several times until no more precipitate was appreciated. All the collected solids were dried in vacuo to give 185.67 g (100%) of the product as a white powder. The compound was used as such in the next synthetic step.

¹H-NMR (300 MHz, DMSO-d₆): δ [ppm]=0.98-1.10 (m, 4H), 2.02-2.17 (m, 1H), 4.31 (d, 2H), 4.51 (d, 3H), 4.65 (d, 3H), 5.32 (s, 6H).

LC-MS (Method 3): Rₜ=0.099 min. MS (Mass method 1): m/z=223 (M)⁺

### Intermediate 86

### tert-Butyl (2-cyclopropyl-2-oxoethyl)carbamate

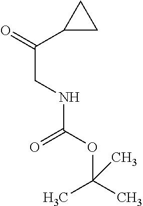

185.67 g (0.61 mol) of 1-(2-cyclopropyl-2-oxoethyl)-3,5,7-triaza-1-azoniatricyclo[3.3.1.1^{3,7}]decane bromide was dissolved in 0.60 L of ethanol and 0.20 L of hydrochloric acid (conc). and the mixture was refluxed for 30 min. The resulting precipitate was filtered off, washed with ethanol and discarded. The filtrates were evaporated and dried to yield the corresponding crude ammonium salt as a thick oil. Next, 0.10 L (0.73 mol) of triethylamine and 0.14 L (0.61 mol) of di-tert-butyl dicarbonate were added to a solution of the aforementioned crude ammonium salt in 1.3 L of dichloromethane and the mixture was stirred at room temperature for 12 hours. Water was added to the reaction and the layers were separated. The organic layer was dried over magnesium sulfate, filtered and evaporated. The corresponding residue was purified by flash chromatography on silica gel eluting with heptane and ethyl acetate. 49.00 g (40%) of the product were isolated as a dark brown oil.

¹H-NMR (300 MHz, DMSO-d₆): δ [ppm]=0.78-0.85 (m, 1H), 0.86-0.94 (m, 2H), 1.38 (s, 9H), 1.99-2.11 (m, 1H), 3.88 (d, 2H), 7.03 (bp, 1H).

LC-MS (Method 3): Rₜ=0.630 min. MS (Mass method 1): m/z=222 (M+Na)⁺

### Intermediate 87

### rac-tert-butyl [(4-cyclopropyl-2,5-dioxoimidazolidin-4-yl)methyl]carbamate

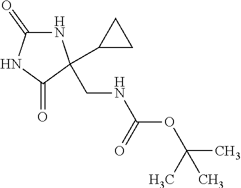 [SMILES: CC(C)(C)OC(=O)NCC1(C2CC2)NC(=O)NC1=O]

To a stirring solution of 50.63 g (526.97 mmol) of ammonium carbonate and 7.51 g (140.52 mmol) of ammonium chloride in 40 mL water were added 7.00 g (35.13 mmol) of tert-butyl (2-cyclopropyl-2-oxoethyl)carbamate in 40 mL of ethanol. After 15 min, 10.29 g (158.09 mmol) of potassium cyanide were added and the mixture was heated up to 60° C. for 16 hours in a sealed pressure flask. The yellow solution was concentrated until only a small fraction of water remained (a white precipitate appeared). Then, more water was added and the suspension was allowed to stand at 0° C. for 1 hour. After that time, the resulting solid was filtered off and washed with water and diethyl ether to give 5.04 g (53%) of the product as a beige powder. The compound was used as such in the next step.

¹H-NMR (300 MHz, DMSO-d₆): δ [ppm]=0.01-0.14 (m, 1H), 0.23-0.34 (m, 1H), 0.35-0.46 (m, 2H), 0.97-1.10 (m, 1H), 1.36 (s, 9H), 3.25-3.32 (m, 2H), 6.78 (t, 1H), 7.36 (s, 1H), 10.46 (bp, 1H)

LC-MS (Method 3): Rₜ=0.388 min. MS (Mass method 1): m/z=214 (M−tBu+H)⁺

### Intermediate 88

### Ethyl 5-isopropyl-1,3-oxazole-4-carboxylate

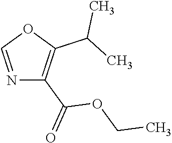

A solution of 2.47 mL (26.67 mmol) of 2-methylpropanoic acid and 5.19 g (32.00 mmol) of 1,1′-carbonyldiimidazole in 30 mL of tetrahydrofuran was stirred for 3 hours at room temperature. After that time, a solution of 3.21 mL (29.34 mmol) of ethyl isocyanoacetate in 35 mL of tetrahydrofuran and a solution of 26.67 mL (26.67 mmol) of 1M lithium bis(trimethylsilyl)amide in tetrahydrofuran were added dropwise at 0° C. When the addition was complete the resulting mixture was stirred at room temperature for 16 hours. The reaction was concentrated in vacuo and extracted with ethyl acetate, the combined organic layers were filtered over magnesium sulfate and concentrated. The resulting residue was purified by flash chromatography on silica gel eluting with heptane/dichloromethane mixtures to give 2.26 g (46%) of the product as a brown oil.

¹H-NMR (300 MHz, DMSO-d₆): δ [ppm]=1.23 (d, 6H), 3.62-3.75 (m, 1H), 4.26 (q, 2H), 8.34 (s, 1H)

LC-MS (Method 3): Rₜ=0.708 min. MS (Mass method 1): m/z=184 (M+H)⁺

### Intermediate 89

### tert-Butyl (3-methyl-2-oxobutyl)carbamate

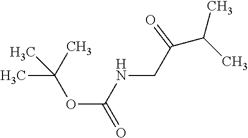

2.26 g (12.39 mmol) of ethyl 5-isopropyl-1,3-oxazole-4-carboxylate was suspended in 40 mL of 6N hydrochloric acid(aq.) and the mixture was refluxed for 12 hours. Concentration of the reaction under reduced pressure gave 1.69 g (99%) of the intermediate amino ketone hydrochloride as a brown oil. A solution of 1.69 g (12.28 mmol) of the latter compound and 3.10 mL (13.51 mmol) of di-tert-butyl dicarbonate were dissolved in 30 mL of dichloromethane and 4.28 mL (30.70 mmol) of triethylamine was added, allowing the mixture to stir at room temperature for 12 hours. The crude was diluted with ethyl acetate, washed with water, dried over magnesium sulfate, filtered and concentrated under vacuum. The resulting residue was purified by flash chromatography on silica gel eluting with heptane/ethyl acetate mixtures to provide 1.96 g (79%) of the product as an oil.

¹H-NMR (300 MHz, DMSO-d₆): δ [ppm]=0.99 (d, 6H), 1.37 (s, 9H), 2.59-2.73 (m, 1H), 3.82 (d, 2H), 6.96 (t, 1H).

LC-MS (Method 3): Rₜ=0.739 min. MS (Mass method 1): m/z=224 (M+Na)⁺

### Intermediate 90

### rac-tert-butyl [(4-isopropyl-2,5-dioxoimidazolidin-4-yl)methyl]carbamate

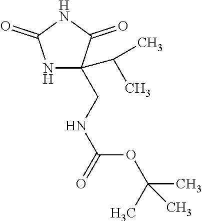 [SMILES: CC(C)C1(CNC(=O)OC(C)(C)C)NC(=O)NC1=O]

A solution of 1.93 g (9.59 mmol) of tert-butyl (3-methyl-2-oxobutyl)carbamate in 15 mL of ethanol was added to a solution of 1.25 g (19.18 mmol) of potassium cyanide and 9.21 g (95.89 mmol) of ammonium carbonate in 15 mL of water into a pressure flask, the container was sealed and the mixture was stirred at 60° C. for 24 hours. After that time, the solvent was partially removed and the resulting precipitate was filtered off to give 1.74 g (67%) of the product as a withe solid. The compound was used as such in the next step.

¹H-NMR (300 MHz, DMSO-d₆): δ [ppm]=0.79 (d, 3H), 0.87 (d, 3H), 1.36 (s, 9H), 1.82-1.95 (m, 1H), 3.12-3.30 (m, 2H), 6.70 (bp, 1H), 7.38 (s, 1H), 10.53 (s, 1H).

LC-MS (Method 3): Rₜ=0.489 min. MS (Mass method 1): m/z=216 (M−t-Bu+H)⁺

### Intermediate 91

### rac-5-(aminomethyl)-5-isopropylimidazolidine-2,4-dione hydrochloride

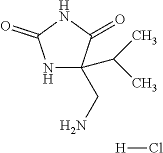

1.74 g (6.41 mmol) of rac-tert-butyl [(4-isopropyl-2,5-dioxoimidazolidin-4-yl)methyl]carbamate was dissolved in 20 mL of dichloromethane and 8.02 mL (32.06 mmol) of 4M hydrochloric acid in dioxane were added and the resulting suspension was stirred at room temperature for 12 hours. After that time, the solvent was in vacuo to give 1.10 g (83%) of the product a solid. The compound was used as such in the next step.

LC-MS (Method 3): Rₜ=0.125 min. MS (Mass method 1): m/z=172 (M+H)⁺

### Intermediate 92

### rac-5-(aminomethyl)-5-cyclobutylimidazolidine-2,4-dione hydrochloride

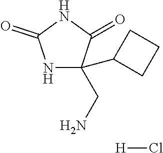

1.53 g (8.35 mmol) of rac-5-(aminomethyl)-5-cyclobutylimidazolidine-2,4-dione was dissolved in 40 mL of dichloromethane and 4.17 mL (16.70 mmol) of 4M hydrochloric acid in dioxane were added and the resulting suspension was stirred at room temperature for 12 hours. After that time, the solvent was removed in vacuo to give 1.76 g (96%) of the product as a solid. The compound was used as such in the next step.

¹H-NMR (300 MHz, DMSO-d₆): δ [ppm]=1.68-1.82 (m, 4H), 1.87-1.97 (m, 2H), 2.62-2.83 (m, 2H), 2.96-3.06 (m, 1H), 8.20 (bp, 4H), 10.96 (s, 1H).

LC-MS (Method 6): Rₜ=0.496 min. MS (Mass method 1): m/z=184 (M+H)⁺

### Intermediate 93

### Ethyl 3-oxo-2-{2-[4-(trifluoromethyl)phenyl]hydrazinylidene}propanoate

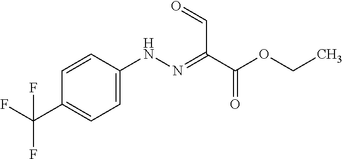 [SMILES: CCOC(=O)/C(C=O)=N/Nc1ccc(C(F)(F)F)cc1]

0.48 g (6.98 mmol) of sodium nitrite in 10 mL of water was added drop wise to solution of 1.13 g (6.98 mmol) of 4-aminobenzotrifluoride in 3 mL of concentrated hydrochloric acid and 5 mL of water at 0° C. and the mixture was stirred for 10 min under the same conditions. After that time, the resulting solution was added drop wise to a solution of 1.00 g (6.98 mmol) of ethyl (2E)-3-(dimethylamino)prop-2-enoate and 1.03 g (10.50 mmol) of potassium acetate in 15 mL of ethanol at 0° C. and then mixture was further stirred at room temperature for 12 hours. The ethanolic fraction of the solvent was removed and the resulting residue was diluted with ethyl acetate and washed with water. The organic layer was dried over magnesium sulfate, filtered and concentrated giving 2.00 g (99%) of the crude product as a deep red thick oil which solidified upon standing. The compound was used as such in the next step.

LC-MS (Method 3): Rₜ=1.022 min. MS (Mass method 1): m/z=289 (M+H)⁺

### Intermediate 94

### Ethyl 3-(hydroxyimino)-2-{2-[4-(trifluoromethyl)phenyl]hydrazinylidene}propanoate

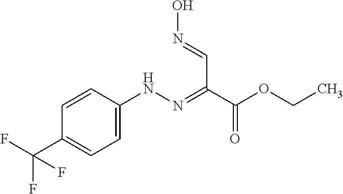 [SMILES: CCOC(=O)C(/C=N/O)=N/Nc1ccc(C(F)(F)F)cc1]

2.00 g (6.94 mmol) of ethyl 3-oxo-2-{2-[4-(trifluoromethyl)phenyl]hydrazinylidene}propanoate, 0.58 g (8.33 mmol) of hydroxylamine hydrochloride and 1.70 g (17.30 mmol) potassium acetate were mixed in 30 mL of ethanol and the mixture was heated up to 78° C. for 1 hour. The resulting red suspension was cooled down to room temperature and 20 mL of water was added. The reaction was extracted with ethyl acetate and the combined organic layers were dried over magnesium sulfate, filtered and concentrated to give 1.60 g (76%) of the crude product as a thick red oil. The compound was used as such in the next step.

LC-MS (Method 3): Rₜ=0.934 min. MS (Mass method 1): m/z=304 (M+H)⁺

### Intermediate 95

### Ethyl 2-[4-(trifluoromethyl)phenyl]-2H-1,2,3-triazole-4-carboxylate

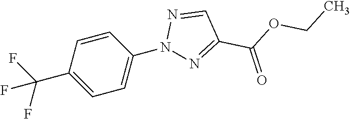 [SMILES: CCOC(=O)c1cnn(-c2ccc(C(F)(F)F)cc2)n1]

1.60 g (5.28 mmol) of ethyl 3-(hydroxyimino)-2-{2-[4-(trifluoromethyl)phenyl]hydrazinylidene}propanoate were dissolved in 20 mL of acetic anhydride and the mixture was stirred at 140° C. for 1 hour. Upon cooling, 20 mL of water were added and the reaction was extracted with dichloromethane. The combined organic layers were dried over magnesium sulfate, filtered and concentrated to give a residue which was further purified by flash chromatography on silica gel using heptane/dichloromethane mixtures to afford 0.47 g (31%) of the product as a yellow oil.

¹H-NMR (300 MHz, DMSO-d₆): δ [ppm]=1.35 (t, 3H), 4.41 (q, 2H), 8.00 (d, 2H), 8.29 (d, 2H), 8.68 (s, 1H).

LC-MS (Method 3): Rₜ=1.018 min. MS (Mass method 1): m/z=286 (M+H)⁺

### Intermediate 96

### 2-[4-(trifluoromethyl)phenyl]-2H-1,2,3-triazole-4-carboxylic acid

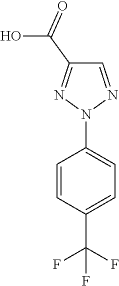 [SMILES: O=C(O)c1cnn(-c2ccc(C(F)(F)F)cc2)n1]

83 mg (1.98 mmol) of lithium hydroxide monohydrate was added to a solution of 470 mg (1.68 mmol) of ethyl 2-[4-(trifluoromethyl)phenyl]-2H-1,2,3-triazole-4-carboxylate in 8 mL of tetrahydrofuran and 4 mL of water and the mixture was stirred at room temperature for 2 hours. After that time, pH was set to 3-4 by addition of 2N hydrochloric acid(aq.) and the solvent was totally removed by rotary evaporation. 420 mg (99%) of the crude product was then isolated as a yellow powder. The compound was used as such in the next synthetic step.

¹H-NMR (300 MHz, DMSO-d₆): δ [ppm]=7.97 (d, 2H), 8.25 (d, 2H), 8.37 (s, 1H).

LC-MS (Method 3): Rₜ=0.753 min. MS (Mass method 1): m/z=258 (M+H)⁺

### Intermediate 97

### Ethyl 2-[2-(4-fluorophenyl)hydrazinylidene]-3-oxopropanoate

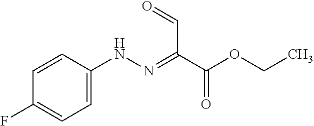 [SMILES: CCOC(=O)/C(C=O)=N/Nc1ccc(F)cc1]

4-Fluoroaniline (3 g, 27 mmol) were treated with 30 ml of water and 6 ml of concentrated hydrochloric acid at 0° C. Sodium nitrite (1.863 g, 27 mmol) was dissolved in 15 ml of water and this solution was added dropwise to the cooled 4-fluoroaniline solution. The mixture was stirred for five minutes at 0° C. before being added dropwise at 0° C. to a mixture of ethyl 3-(dimethylamino)acrylate (4.252 g, 29.7 mmol) and potassium acetate (3,974 g, 40.5 mmol) in 45 ml of ethanol. The reaction mixture was stirred for 10 minutes at room temperature and then diluted with ethyl acetate. The mixture was washed twice with water and once with saturated aqueous sodium chloride solution. The organic layer was dried over sodium sulfate, filtered, Isolute® was added, and the solvent was evaporated on a rotary evaporator. The crude product was purified by column chromatography (Machine: Biotage® Isolera One; Column: Biotage® SNAP Ultra 100 g; Gradient: Cyclohexane/Ethyl acetate, 5% Ethyl acetate→40% Ethyl acetate; Flow: 100 ml/min). 3.89 g (50% yield, 83% purity) of the title compound were obtained.

LC-MS (Method 7): Rₜ=1.81 min; MS (ESIpos): m/z=239 [M+H]⁺

### Intermediate 98

### Ethyl 2-[(4-fluorophenyl)hydrazono]-3-(hydroxyimino)propanoate

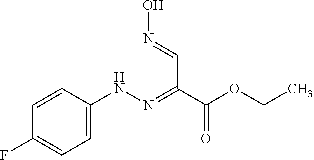 [SMILES: CCOC(=O)C(/C=N/O)=N/Nc1ccc(F)cc1]

Ethyl 2-[(4-fluorophenyl)hydrazono]-3-oxopropanoate (3.885 g, 83% purity, 13.37 mmol) was dissolved in 41 ml of ethanol and treated with hydroxylamine hydrochloride (1.115 g, 16.05 mmol) and potassium acetate (3.281 g, 33.43 mmol). The mixture was stirred over night at room temperature, diluted with ethyl acetate, washed twice with water and once with saturated aqueous sodium chloride solution. The organic layer was dried over sodium sulfate and filtered. Isolute® was added and the solvent was removed on a rotary evaporator. The crude product was purified by column chromatography (Machine: Biotage® Isolera One; Column: Biotage® SNAP Ultra 100 g; Gradient: Cyclohexane/Ethyl acetate, 5% Ethyl acetate→40% Ethyl acetate; Flow: 100 ml/min). 3.07 g (91% yield, 100% purity) of the title compound were obtained.

LC-MS (Method 7): Rₜ=1.72 min; MS (ESIpos): m/z=254 [M+H]⁺

### Intermediate 99

### Ethyl 2-(4-fluorophenyl)-2H-1,2,3-triazole-4-carboxylate

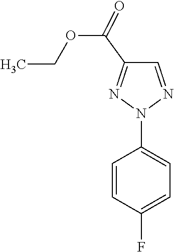 [SMILES: CCOC(=O)c1cnn(-c2ccc(F)cc2)n1]

Ethyl 2-[(4-fluorophenyl)hydrazono]-3-(hydroxyimino)propanoate (3.07 g, 12.12 mmol) was dissolved in 45 ml of acetic anhydride. The solution was stirred at 60° C. for 1.5 hours before being treated with water. The mixture was extracted with ethyl acetate and the organic layer was dried over sodium sulfate. Isolute® was added and the solvent was removed on a rotary evaporator. The residue was purified by column chromatography (Machine: Biotage® Isolera One; Column: Biotage® SNAP Ultra 100 g; Gradient: Cyclohexane/Ethyl acetate, 5% Ethyl acetate→40% Ethyl acetate; Flow: 100 ml/min). 1.84 g (65% yield, 100% purity) of the title compound were obtained.

LC-MS (Method 7): Rₜ=1.95 min; MS (ESIpos): m/z=236 [M+H]⁺

### Intermediate 100

### 2-(4-fluorophenyl)-2H-1,2,3-triazole-4-carboxylic acid

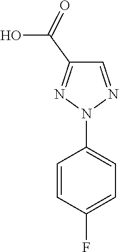 [SMILES: O=C(O)c1cnn(-c2ccc(F)cc2)n1]

Ethyl 2-(4-fluorophenyl)-2H-1,2,3-triazole-4-carboxylate (1.38 g, 783 mmol) was dissolved in THF, treated with lithium hydroxide solution (1N in water, 7.83 ml, 7.83 mmol), and the mixture was stirred at room temperature for four hours. The THF was removed on a rotary evaporator and the residue was acidified with excess 2N aqueous hydrochloric acid. The precipitate was filtered off, washed with water and dried in vacuo. 1.38 g (85% yield, 100% purity) of the title compound were obtained.

LC-MS (Method 7): Rₜ=1.23 min; MS (ESIpos): m/z=206 [M−H]⁻

### Intermediate 101

### Ethyl 2-[2-(3-fluorophenyl)hydrazinylidene]-3-oxopropanoate

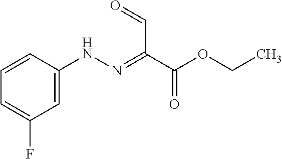 [SMILES: CCOC(=O)/C(C=O)=N/Nc1cccc(F)c1]

0.96 g (14.00 mmol) of sodium nitrite in 10 mL of water was added dropwise to a cold solution of 1.30 mL (14.00 mmol) of 3-fluoroaniline in 3 mL of concentrated hydrochloric acid and 10 mL of water at 0° C. and the mixture was stirred for 5 min under the same conditions. This solution was added drop wise to a cold solution of 2.00 g (14.00 mmol) of (2E)-3-(dimethylamino)prop-2-enoate and 2.06 g (21.00 mmol) of potassium acetate in 20 mL of ethanol and the reaction was stirred at room temperature for 14 hours. The mixture was then diluted with ethyl acetate and the dark red solution was washed with water and brine. The aqueous phase was dried over magnesium sulfate, filtered and concentrated in vacuo to afford 3.19 g (96%) of the product as a red solid, which required no further purification.

LC-MS (Method 3): Rₜ=0.920 min. MS (Mass method 1): m/z=239 (M+H)⁺

### Intermediate 102

### Ethyl 2-[2-(3-fluorophenyl)hydrazinylidene]-3-(hydroxyimino)propanoate

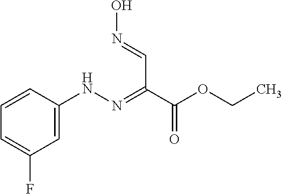 [SMILES: CCOC(=O)C(/C=N/O)=N/Nc1cccc(F)c1]

3.20 g (13.40 mmol) of ethyl 2-[2-(3-fluorophenyl)hydrazinylidene]-3-oxopropanoate, 0.93 g (13.40 mmol) of hydroxylamine hydrochloride and 3.30 g (33.60 mmol) of potassium acetate were mixed in 40 mL of ethanol and heated to 78° C. for 30 min. The reaction was cooled to room temperature and 20 mL of water were added. The mixture was extracted with ethyl acetate and the combined organic extracts were dried over magnesium sulfate, filtered and concentrated to give 3.39 g (99%) of the product as a tan thick oil. The compound was used in the next step without further purification.

LC-MS (Method 3): Rₜ=0.901 min. MS (Mass method 1): m/z=254 (M+H)⁺

### Intermediate 103

### Ethyl 2-(3-fluorophenyl)-2H-1,2,3-triazole-4-carboxylate

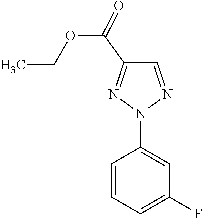 [SMILES: CCOC(=O)c1cnn(-c2cccc(F)c2)n1]

3.30 g (13.00 mmol) of ethyl 2-[2-(3-fluorophenyl)hydrazinylidene]-3-(hydroxyimino) propanoate were dissolved in 30 mL of acetic anhydride and the reaction was heated at 140° C. for 1 hour. The mixture was then cooled down to room temperature and 10 mL of water was added. The dark solution was extracted with dichloromethane and the combined organic layers were dried over magnesium sulfate, filtered and concentrated. The resulting residue was purified by flash chromatography on silica gel eluting with heptane/dichloromethane mixtures to afford 0.30 g (10%) of the product as a yellow solid.

¹H-NMR (300 MHz, DMSO-d₆): δ [ppm]=1.34 (t, 3H), 4.39 (q, 2H), 7.38 (t, 1H), 7.67 (q, 1H), 7.86 (d, 1H), 7.94 (d, 1H), 8.63 (s, 1H).

LC-MS (Method 3): Rₜ=1.004 min. MS (Mass method 1): m/z=236 (M+H)⁺

### Intermediate 104

### 2-(3-fluorophenyl)-2H-1,2,3-triazole-4-carboxylic acid

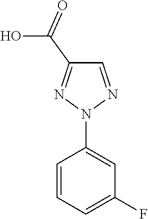 [SMILES: O=C(O)c1cnn(-c2cccc(F)c2)n1]

107 mg (2.55 mmol) of lithium hydroxide monohydrate were added to a solution of 300 mg (1.28 mmol) of ethyl 2-(3-fluorophenyl)-2H-1,2,3-triazole-4-carboxylate in 4 mL of tetrahydrofuran aid 2 mL of water and the reaction was allowed to stir at room temperature for 2 hours. The pH was adjusted to 4-5 by addition of 2N hydrochloric acid(aq.) and the solvent was totally removed to yield 260 mg (98%) of the product as a yellow powder. The compound was used in the next step without further purification.

LC-MS (Method 3): Rₜ=0.667 min. MS (Mass method 1): m/z=208 (M+H)⁺

### Intermediate 105

### Ethyl 2-{[3-fluoro-4-(trifluoromethyl)phenyl]hydrazono}-3-oxopropanoate

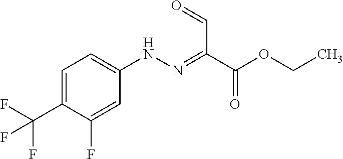 [SMILES: CCOC(=O)/C(C=O)=N/Nc1ccc(C(F)(F)F)c(F)c1]

0.48 g (6.98 mmol) of sodium nitrite in 5 mL of water were added dropwise to a cold solution of 1.25 g (6.98 mmol) of 3-fluoro-4-(trifluoromethyl)aniline in 3 mL of concentrated hydrochloric acid and 5 mL of water at 0° C. and the mixture was stirred for 5 min under the same conditions. This solution was added drop wise to a cold solution of 1.00 g (6.98 mmol) of (2E)-3-(dimethylamino)prop-2-enoate and 1.03 g (10.47 mmol) of potassium acetate in 15 mL of ethanol and the reaction was stirred at room temperature for 14 hours. The mixture was then diluted with ethyl acetate and the dark red solution was washed with water and brine. The aqueous phase was dried over magnesium sulfate, filtered and concentrated in vacuo to afford 2.06 g (96%) of the product as a deep red thick oil, which required no further purification.

¹H-NMR (300 MHz, DMSO-d₆): δ [ppm]=1.31 (t, 3H), 4.26-4.40 (q, 2H), 7.49-7.72 (m, 2H), 7.76-7.87 (m, 1H), 9.51 (s, 1H), 9.86 (s, 1H).

LC-MS (Method 3): Rₜ=1.175 min. MS (Mass method 1): m/z=307 (M+H)⁺

### Intermediate 106

### Ethyl 2-{2-[3-fluoro-4-(trifluoromethyl)phenyl]hydrazinylidene}-3-(hydroxyimino)propanoate

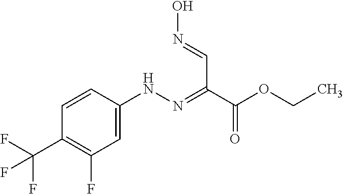 [SMILES: CCOC(=O)C(/C=N/O)=N/Nc1ccc(C(F)(F)F)c(F)c1]

2.06 g (6.73 mmol) of ethyl 2-{2-[3-fluoro-4-(trifluoromethyl)phenyl]hydrazinylidene}-3-oxopropanoate, 0.56 g (8.07 mmol) of hydroxylamine hydrochloride and 1.65 g (16.80 mmol) of potassium acetate were mixed in 50 mL of ethanol and heated to 78° C. for 30 min. The reaction was cooled to room temperature and 20 mL of water were added. The mixture was extracted with ethyl acetate and the combined organic extracts were dried over magnesium sulfate, filtered and concentrated to give 2.10 g (97%) of the product as a red thick oil. The compound was used in the next step without further purification.

¹H-NMR (300 MHz, DMSO-d₆): δ [ppm]=1.30 (t, 3H), 4.27 (q, 2H), 7.16-7.33 (m, 2H), 7.77 (t, 1H), 8.27 (s, 1H), 12.32 (s, 1H), 12.54 (s, 1H).

LC-MS (Method 3): Rₜ=1.009 min. MS (Mass method 1): m/z=322 (M+H)⁺

### Intermediate 107

### Ethyl 2-[3-fluoro-4-(trifluoromethyl)phenyl]-2H-1,2,3-triazole-4-carboxylate

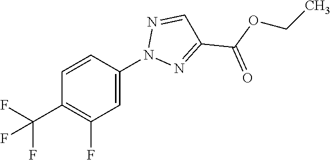 [SMILES: CCOC(=O)c1cnn(-c2ccc(C(F)(F)F)c(F)c2)n1]

2.20 g (6.85 mmol) of ethyl 2-{2-[3-fluoro-4-(trifluoromethyl)phenyl]hydrazinylidene}-3-(hydroxyimino)propanoate were dissolved in 50 mL of acetic anhydride and the reaction was heated at 140° C. for 1 hour. The mixture was then cooled down to room temperature and 10 mL of water was added. The dark solution was extracted with dichloromethane and the combined organic layers were dried over magnesium sulfate, filtered and concentrated. The resulting residue was purified by flash chromatography on silica gel eluting with heptane/dichloromethane mixtures to afford 1.30 g (63%) of the product as a yellow solid.

¹H-NMR (300 MHz, DMSO-d₆): δ [ppm]=1.35 (t, 3H), 4.41 (q, 1H), 8.00-8.23 (m, 3H), 8.73 (s, 1H)

LC-MS (Method 3): Rₜ=1.300 min. MS (Mass method 1): m/z=304 (M+H)⁺

### Intermediate 108

### 2-[3-fluoro-4-(trifluoromethyl)phenyl]-2H-1,2,3-triazole-4-carboxylic acid

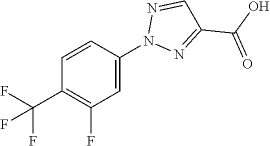 [SMILES: O=C(O)c1cnn(-c2ccc(C(F)(F)F)c(F)c2)n1]

0.26 g (5.14 mmol) of lithium hydroxide monohydrate were added to a solution of 1.30 g (4.29 mmol) of ethyl 2-[3-fluoro-4-(trifluoromethyl)phenyl]-2H-1,2,3-triazole-4-carboxylate in 20 mL of tetrahydrofuran and 10 mL of water and the reaction was allowed to stir at room temperature for 2 hours. The pH was adjusted to 4-5 by addition of 2N hydrochloric acid(aq.) and the solvent was totally removed to yield 1.18 g (99%) of the product as a yellow powder. The compound was used in the next step without further purification.

¹H-NMR (300 MHz, DMSO-d₆): δ [ppm]=7.94-7.86 (m, 3H), 8.09 (s, 1H).

LC-MS (Method 3): Rₜ=0.767 min. MS (Mass method 1): m/z=276 (M+H)⁺

### Intermediate 109

### Ethyl 3-oxo-2-(2-phenylhydrazinylidene)propanoate

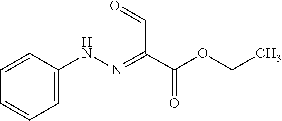 [SMILES: CCOC(=O)/C(C=O)=N/Nc1ccccc1]

0.96 g (13.96 mmol) of sodium nitrite in 10 mL of water were added dropwise to a cold solution of 1.3 mL (13.96 mmol) of aniline in 3 mL of concentrated hydrochloric acid and 10 mL of water at 0° C. and the mixture was stirred for 5 min under the same conditions. This solution was added drop wise to a cold solution of 2.00 g (14.00 mmol) of (2E)-3-(dimethylamino)prop-2-enoate aid 2.06 g (21.00 mmol) of potassium acetate in 15 mL of ethanol and the reaction was stirred at room temperature for 14 hours. The mixture was then diluted with ethyl acetate and the dark red solution was washed with water and brine. The aqueous phase was dried over magnesium sulfate, filtered and concentrated in vacuo to afford 2.86 g (93%) of the product as a red solid, which required no further purification.

LC-MS (Method 3): Rₜ=0.852 min. MS (Mass method 1): m/z=221 (M+H)⁺

### Intermediate 110

### Ethyl 3-(hydroxyimino)-2-(2-phenylhydrazinylidene)propanoate

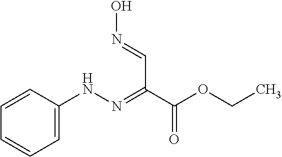 [SMILES: CCOC(=O)C(/C=N/O)=N/Nc1ccccc1]

2.86 g (13.00 mmol) of ethyl 3-oxo-2-(2-phenylhydrazinylidene)propanoate, 1.08 g (15.60 mmol) of hydroxylamine hydrochloride and 3.19 g (32.50 mmol) of potassium acetate were mixed in 30 mL of ethanol and heated to 78° C. for 30 min. The reaction was cooled to room temperature aid 20 mL of water were added. The mixture was extracted with ethyl acetate and the combined organic extracts were dried over magnesium sulfate, filtered and concentrated to give 1.20 g (34%) of the product as a red thick oil. The compound was used in the next step without further purification.

LC-MS (Method 3): Rₜ=0.820 min. MS (Mass method 1): m/z=236 (M+H)⁺

### Intermediate 111

### Ethyl 2-phenyl-2H-1,2,3-triazole-4-carboxylate

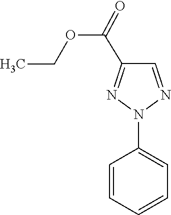 [SMILES: CCOC(=O)c1cnn(-c2ccccc2)n1]

1.20 g (5.10 mmol) of ethyl 3-(hydroxyimino)-2-(2-phenylhydrazinylidene)propanoate were dissolved in 20 mL of acetic anhydride and the reaction was heated at 140° C. for 1 hour. The mixture was then cooled down to room temperature and 10 mL of water was added. The dark solution was extracted with dichloromethane and the combined organic layers were dried over magnesium sulfate, filtered and concentrated. The resulting residue was purified by flash chromatography on silica gel eluting with heptane/dichloromethane mixtures to afford 0.86 g (78%) of the product as a yellow solid.

¹H-NMR (300 MHz, DMSO-d₆): δ [ppm]=1.34 (t, 3H), 4.39 (q, 2H), 7.47-7.55 (m, 1H), 7.62 (t, 2H), 8.06 (d, 2H), 8.59 (s, 1H).

LC-MS (Method 3): Rₜ=0.918 min. MS (Mass method 1): m/z=218 (M+H)⁺

### Intermediate 112

### 2-phenyl-2H-1,2,3-triazole-4-carboxylic acid

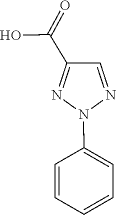 [SMILES: O=C(O)c1cnn(-c2ccccc2)n1]

332 mg (7.92 mmol) of lithium hydroxide monohydrate were added to a solution of 860 mg (3.96 mmol) of ethyl 2-phenyl-2H-1,2,3-triazole-4-carboxylate in 10 mL of tetrahydrofuran and 5 mL of water and the reaction was allowed to stir at room temperature for 2 hours. The pH was adjusted to 4-5 by addition of 2N hydrochloric acid(aq.) and the solvent was totally removed to yield 740 mg (99%) of the product as a yellow powder. The compound was used in the next step without further purification.

¹H-NMR (300 MHz, DMSO-d₆): δ [ppm]=7.39 (t, 1H), 7.55 (t, 2H), 7.97-8.05 (m, 3H).

LC-MS (Method 3): Rₜ=0.615 min. MS (Mass method 1): m/z=190 (M+H)⁺

### Intermediate 113

### Ethyl 2-[2-(3,4-difluorophenyl)hydrazinylidene]-3-oxopropanoate

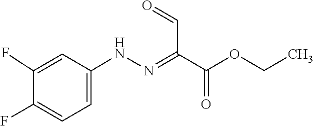 [SMILES: CCOC(=O)/C(C=O)=N/Nc1ccc(F)c(F)c1]

0.96 g (14.00 mmol) of sodium nitrite in 10 mL of water were added dropwise to a cold solution of 1.4 mL (14.00 mmol) of 3,4-difluoroaniline in 4 mL of concentrated hydrochloric acid and 10 mL of water at 0° C. and the mixture was stirred for 5 min under the same conditions. This solution was added drop wise to a cold solution of 2.00 g (14.00 mmol) of (2E)-3-(dimethylamino)prop-2-enoate and 2.06 g (21.00 mmol) of potassium acetate in 15 mL of ethanol and the reaction was stirred at room temperature for 14 hours. The mixture was then diluted with ethyl acetate and the dark red solution was washed with water and brine. The aqueous phase was dried over magnesium sulfate, filtered and concentrated in vacuo to afford 3.57 g (99%) of the product as a thick red oil, which required no further purification.

LC-MS (Method 3): Rₜ=0.858 min. MS (Mass method 1): m/z=257 (M+H)⁺

### Intermediate 114

### Ethyl 2-[2-(3,4-difluorophenyl)hydrazinylidene]-3-(hydroxyimino)propanoate

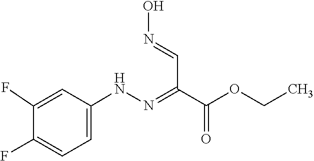 [SMILES: CCOC(=O)C(/C=N/O)=N/Nc1ccc(F)c(F)c1]

3.57 g (13.90 mmol) of ethyl 2-[2-(3,4-difluorophenyl)hydrazinylidene]-3-oxopropanoate, 1.16 g (16.70 mmol) of hydroxylamine hydrochloride and 3.42 g (34.80 mmol) of potassium acetate were mixed in 40 mL of ethanol and heated to 78° C. for 30 min. The reaction was cooled to room temperature and 20 mL of water were added. The mixture was extracted with ethyl acetate and the combined organic extracts were dried over magnesium sulfate, filtered and concentrated to give 3.77 g (99%) of the product as a red thick oil. The compound was used in the next step without further purification.

LC-MS (Method 3): Rₜ=0.846 min. MS (Mass method 1): m/z=272 (M+H)⁺

### Intermediate 115

### Ethyl 2-(3,4-difluorophenyl)-2H-1,2,3-triazole-4-carboxylate

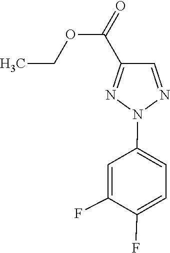 [SMILES: CCOC(=O)c1cnn(-c2ccc(F)c(F)c2)n1]

3.77 g (13.90 mmol) of ethyl 2-[2-(3,4-difluorophenyl)hydrazinylidene]-3-(hydroxyimino)propanoate were dissolved in 40 mL of acetic anhydride and the reaction was heated at 140° C. for 1 hour. The mixture was then cooled down to room temperature and 10 mL of water was added. The dark solution was extracted with dichloromethane and the combined organic layers were dried over magnesium sulfate, filtered and concentrated. The resulting residue was purified by flash chromatography on silica gel eluting with heptane/dichloromethane mixtures to afford 1.00 g (28%) of the product as a yellow solid.

¹H-NMR (300 MHz, DMSO-d₆): δ [ppm]=1.34 (t, 3H), 4.39 (q, 2H), 7.71 (q, 1H), 7.93 (d, 1H), 8.12 (td, 1H), 8.63 (s, 1H).

LC-MS (Method 3): Rₜ=0.920 min. MS (Mass method 1): m/z=254 (M+H)⁺

### Intermediate 116

### 2-(3,4-difluorophenyl)-2H-1,2,3-triazole-4-carboxylic acid

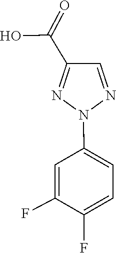 [SMILES: O=C(O)c1cnn(-c2ccc(F)c(F)c2)n1]

199 mg (4.74 mmol) of lithium hydroxide monohydrate were added to a solution of 1000 mg (3.95 mmol) of ethyl 2-(3,4-difluorophenyl)-2H-1,2,3-triazole-4-carboxylate in 8 mL of tetrahydrofuran and 4 mL of water and the reaction was allowed to stir at room temperature for 2 hours. The pH was adjusted to 4-5 by addition of 2N hydrochloric acid(aq.) and the solvent was totally removed to yield 880 mg (99%) of the product as a yellow powder. The compound was used in the next step without further purification.

¹H-NMR (300 MHz, DMSO-d₆): δ [ppm]=7.64 (q, 1H), 7.84 (d, 1H), 7.94-8.07 (m, 2H).

LC-MS (Method 3): Rₜ=0.636 min. MS (Mass method 1): m/z=226 (M+H)⁺

### Intermediate 117

### Ethyl 2-[2-(4-chlorophenyl)hydrazinylidene]-3-oxopropanoate

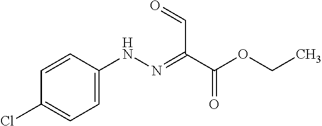 [SMILES: CCOC(=O)/C(C=O)=N/Nc1ccc(Cl)cc1]

0.48 g (6.98 mmol) of sodium nitrite in 5 mL of water were added dropwise to a cold solution of 0.89 g (6.98 mmol) of 4-chloroaniline in 1.5 mL of concentrated hydrochloric acid and 5 mL of water at 0° C. and the mixture was stirred for 5 min under the same conditions. This solution was added drop wise to a cold solution of 1.00 g (6.98 mmol) of (2E)-3-(dimethylamino)prop-2-enoate and 1.03 g (10.50 mmol) of potassium acetate in 15 mL of ethanol and the reaction was stirred at room temperature for 14 hours. The mixture was then diluted with ethyl acetate and the dark red solution was washed with water and brine. The aqueous phase was dried over magnesium sulfate, filtered and concentrated in vacuo to afford 1.58 g (89%) of the product as a thick orange oil, which required no further purification.

LC-MS (Method 3): Rₜ=0.914 min. MS (Mass method 1): m/z=255 (M+H)⁺

### Intermediate 118

### Ethyl 2-[2-(4-chlorophenyl)hydrazinylidene]-3-(hydroxyimino)propanoate

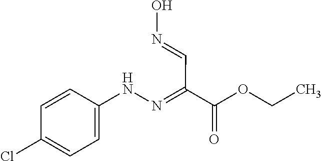 [SMILES: CCOC(=O)C(/C=N/O)=N/Nc1ccc(Cl)cc1]

1.58 g (6.20 mmol) of ethyl 2-[2-(4-chlorophenyl)hydrazinylidene]-3-oxopropanoate, 0.52 g (7.44 mmol) of hydroxylamine hydrochloride and 1.52 g (15.50 mmol) of potassium acetate were mixed in 40 mL of ethanol and heated to 78° C. for 30 min. The reaction was cooled to room temperature and 20 mL of water were added. The mixture was extracted with ethyl acetate and the combined organic extracts were dried over magnesium sulfate, filtered and concentrated to give 1.42 g (85%) of the product as a red thick oil. The compound was used in the next step without further purification.

LC-MS (Method 3): Rₜ=0.889 min. MS (Mass method 1): m/z=270 (M+H)⁺

### Intermediate 119

### Ethyl 2-(4-chlorophenyl)-2H-1,2,3-triazole-4-carboxylate

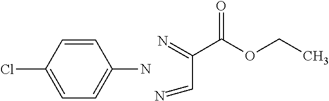

1.42 g (5.27 mmol) of ethyl 2-[2-(4-chlorophenyl)hydrazinylidene]-3-(hydroxyimino) propanoate were dissolved in 15 mL of acetic anhydride and the reaction was heated at 140° C. for 1 hour. The mixture was then cooled down to room temperature and 10 mL of water was added. The dark solution was extracted with dichloromethane and the combined organic layers were dried over magnesium sulfate, filtered and concentrated. The resulting residue was purified by flash chromatography on silica gel eluting with heptane/dichloromethane mixtures to afford 0.56 g (42%) of the product as an orange powder.

¹H-NMR (300 MHz, DMSO-d₆): δ [ppm]=1.35 (t, 3H), 4.40 (q, 2H), 7.69 (d, 2H), 8.09 (d, 2H), 8.62 (s, 1H).

LC-MS (Method 3): Rₜ=0.986 min. MS (Mass method 1): m/z=252 (M+H)⁺

### Intermediate 120

### 2-(4-chlorophenyl)-2H-1,2,3-triazole-4-carboxylic acid

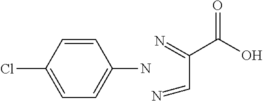

112 mg (2.67 mmol) of lithium hydroxide monohydrate were added to a solution of 561 mg (2.23 mmol) of ethyl 2-(4-chlorophenyl)-2H-1,2,3-triazole-4-carboxylate in 6 mL of tetrahydrofuran and 3 mL of water and the reaction was allowed to stir at room temperature for 2 hours. The pH was adjusted to 4-5 by addition of 2N hydrochloric acid(aq.) and the solvent was totally removed to yield 490 mg (99%) of the product as a light yellow powder. The compound was used in the next step without further purification.

¹H-NMR (300 MHz, DMSO-d₆): δ [ppm]=7.68 (d, 2H), 8.07 (s, 2H), 8.53 (s, 1H).

LC-MS (Method 3): Rₜ=0.704 min. MS (Mass method 1): m/z=224 (M+H)⁺

### Intermediate 121

### Ethyl 2-[2-(3-chlorophenyl)hydrazinylidene]-3-oxopropanoate

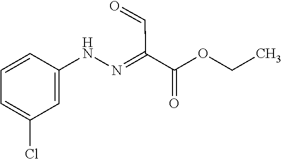 [SMILES: CCOC(=O)/C(C=O)=N/Nc1cccc(Cl)c1]

0.48 g (6.98 mmol) of sodium nitrite in 5 mL of water were added dropwise to a cold solution of 0.74 mL (7.00 mmol) of 3-chloroaniline in 1.5 mL of concentrated hydrochloric acid and 5 mL of water at 0° C. and the mixture was stirred for 5 min under the same conditions. This solution was added drop wise to a cold solution of 1.00 g (6.98 mmol) of (2E)-3-(dimethylamino)prop-2-enoate and 1.03 g (10.50 mmol) of potassium acetate in 15 mL of ethanol and the reaction was stirred at room temperature for 14 hours. The mixture was then diluted with ethyl acetate and the dark red solution was washed with water and brine. The aqueous phase was dried over magnesium sulfate, filtered and concentrated in vacuo to afford 1.64 g (92%) of the product as a thick dark orange oil, which required no further purification.

LC-MS (Method 3): Rₜ=0.919 min. MS (Mass method 1): m/z=255 (M+H)⁺

### Intermediate 122

### Ethyl 2-[2-(3-chlorophenyl)hydrazinylidene]-3-(hydroxyimino)propanoate

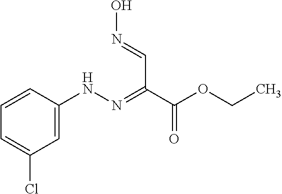 [SMILES: CCOC(=O)C(/C=N/O)=N/Nc1cccc(Cl)c1]

1.64 g (6.44 mmol) of ethyl 2-[2-(3-chlorophenyl)hydrazinylidene]-3-oxopropanoate, 0.54 g (7.73 mmol) of hydroxylamine hydrochloride and 1.58 g (16.10 mmol) of potassium acetate were mixed in 40 mL of ethanol and heated to 78° C. for 30 min. The reaction was cooled to room temperature and 20 mL of water were added. The mixture was extracted with ethyl acetate and the combined organic extracts were dried over magnesium sulfate, filtered and concentrated to give 1.22 g (70%) of the product as a red oil. The compound was used in the next step without further purification.

LC-MS (Method 3): Rₜ=0.895 min. MS (Mass method 1): m/z=270 (M+H)⁺

### Intermediate 123

### Ethyl 2-(3-chlorophenyl)-2H-1,2,3-triazole-4-carboxylate

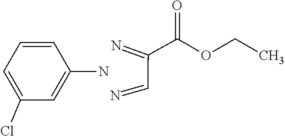

1.22 g (4.53 mmol) of ethyl 2-[2-(3-chlorophenyl)hydrazinylidene]-3-(hydroxyimino) propanoate were dissolved in 15 mL of acetic anhydride and the reaction was heated at 140° C. for 1 hour. The mixture was then cooled down to room temperature and 10 mL of water was added. The dark solution was extracted with dichloromethane and the combined organic layers were dried over magnesium sulfate, filtered and concentrated. The resulting residue was purified by flash chromatography on silica gel eluting with heptane/dichloromethane mixtures to afford 0.47 g (41%) of the product as an orange oil.

¹H-NMR (300 MHz, DMSO-d₆): δ [ppm]=1.35 (t, 3H), 4.40 (q, 2H), 7.57-7.70 (m, 2H), 8.01-8.09 (m, 2H), 8.64 (s, 1H).

LC-MS (Method 3): Rₜ=0.992 min. MS (Mass method 1): m/z=252 (M+H)⁺

### Intermediate 124

### 2-(3-chlorophenyl)-2H-1,2,3-triazole-4-carboxylic acid

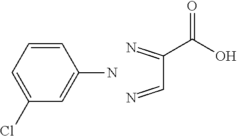

94 mg (2.25 mmol) of lithium hydroxide monohydrate were added to a solution of 472 mg (1.88 mmol) of ethyl 2-(3-chlorophenyl)-2H-1,2,3-triazole-4-carboxylate in 6 mL of tetrahydrofuran and 3 mL of water and the reaction was allowed to stir at room temperature for 2 hours. The pH was adjusted to 4-5 by addition of 2N hydrochloric acid(aq.) and the solvent was totally removed to yield 490 mg (99%) of the product as a light yellow powder. The compound was used in the next step without further purification.

¹H-NMR (300 MHz, DMSO-d₆): δ [ppm]=7.55-7.71 (m, 2H), 8.01-8.09 (m, 2H), 8.56 (s, 1H).

LC-MS (Method 3): Rₜ=0.697 min. MS (Mass method 1): m/z=224 (M+H)⁺

### Intermediate 125

### Ethyl 2-[2-(2-chlorophenyl)hydrazinylidene]-3-oxopropanoate

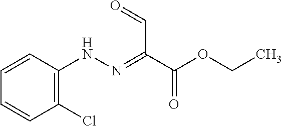 [SMILES: CCOC(=O)/C(C=O)=N/Nc1ccccc1Cl]

0.48 g (6.98 mmol) of sodium nitrite in 5 mL of water were added dropwise to a cold solution of 0.74 mL (7.00 mmol) of 2-chloroaniline in 1.5 mL of concentrated hydrochloric acid and 5 mL of water at 0° C. and the mixture was stirred for 5 min under the same conditions. This solution was added drop wise to a cold solution of 1.00 g (6.98 mmol) of (2E)-3-(dimethylamino)prop-2-enoate and 1.03 g (10.50 mmol) of potassium acetate in 15 mL of ethanol and the reaction was stirred at room temperature for 14 hours. The mixture was then diluted with ethyl acetate and the dark red solution was washed with water and brine. The aqueous phase was dried over magnesium sulfate, filtered and concentrated in vacuo to afford 1.66 g (93%) of the product as a thick dark orange oil, which required no further purification.

LC-MS (Method 3): Rₜ=0.940 min. MS (Mass method 1): m/z=255 (M+H)⁺

### Intermediate 126

### Ethyl 2-[2-(2-chlorophenyl)hydrazinylidene]-3-(hydroxyimino)propanoate

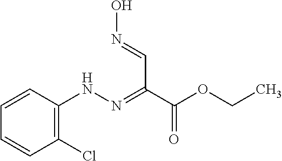 [SMILES: CCOC(=O)C(/C=N/O)=N/Nc1ccccc1Cl]

1.66 g (6.52 mmol) of ethyl 2-[2-(2-chlorophenyl)hydrazinylidene]-3-oxopropanoate, 0.54 g (7.82 mmol) of hydroxylamine hydrochloride and 1.60 g (16.30 mmol) of potassium acetate were mixed in 40 mL of ethanol and heated to 78° C. for 30 min. The reaction was cooled to room temperature and 20 mL of water were added. The mixture was extracted with ethyl acetate and the combined organic extracts were dried over magnesium sulfate, filtered and concentrated to give 1.65 g (94%) of the product as a red oil. The compound was used in the next step without further purification.

LC-MS (Method 3): Rₜ=0.872 min. MS (Mass method 1): m/z=270 (M+H)⁺

### Intermediate 127

### Ethyl 2-(2-chlorophenyl)-2H-1,2,3-triazole-4-carboxylate

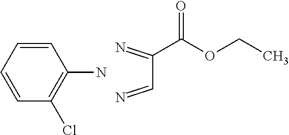

1.65 g (6.11 mmol) of ethyl 2-[2-(2-chlorophenyl)hydrazinylidene]-3-(hydroxyimino) propanoate were dissolved in 20 mL of acetic anhydride and the reaction was heated at 140° C. for 1 hour. The mixture was then cooled down to room temperature and 10 mL of water was added. The dark solution was extracted with dichloromethane and the combined organic layers were dried over magnesium sulfate, filtered and concentrated. The resulting residue was purified by flash chromatography on silica gel eluting with heptane/dichloromethane mixtures to afford 1.10 g (71%) of the product as an orange oil.

¹H-NMR (300 MHz, DMSO-d₆): δ [ppm]=1.34 (t, 3H), 4.39 (q, 2H), 7.58-7.72 (m, 2H), 7.64-7.83 (m, 2H), 8.64 (s, 1H).

LC-MS (Method 3): Rₜ=0.844 min. MS (Mass method 1): m/z=252 (M+H)⁺

### Intermediate 128

### 2-(2-chlorophenyl)-2H-1,2,3-triazole-4-carboxylic acid

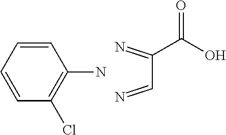

0.22 g (5.24 mmol) of lithium hydroxide monohydrate were added to a solution of 1.10 g (4.37 mmol) of ethyl 2-(2-chlorophenyl)-2H-1,2,3-triazole-4-carboxylate in 6 mL of tetrahydrofuran and 3 mL of water and the reaction was allowed to stir at room temperature for 2 hours. The pH was adjusted to 4-5 by addition of 2N hydrochloric acid(aq.) and the solvent was totally removed to yield 0.97 g (99%) of the product as a yellow powder. The compound was used in the next step without further purification.

¹H-NMR (300 MHz, DMSO-d₆): δ [ppm]=7.55-7.71 (m, 2H), 7.74-7.82 (m, 2H), 8.55 (s, 1H).

LC-MS (Method 3): Rₜ=0.557 min. MS (Mass method 1): m/z=224 (M+H)⁺

### Intermediate 129

### Ethyl 2-[2-(2-methylphenyl)hydrazinylidene]-3-oxopropanoate

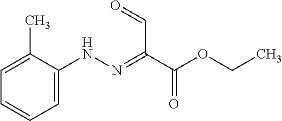 [SMILES: CCOC(=O)/C(C=O)=N/Nc1ccccc1C]

0.48 g (6.98 mmol) of sodium nitrite in 10 mL of water were added dropwise to a cold solution of 0.75 mL (7.00 mmol) of 2-methylaniline in 3 mL of concentrated hydrochloric acid and 10 mL of water at 0° C. and the mixture was stirred for 5 min under the same conditions. This solution was added drop wise to a cold solution of 1.00 g (6.98 mmol) of (2E)-3-(dimethylamino)prop-2-enoate and 1.03 g (10.50 mmol) of potassium acetate in 15 mL of ethanol and the reaction was stirred at room temperature for 14 hours. The mixture was then diluted with ethyl acetate and the dark red solution was washed with water and brine. The aqueous phase was dried over magnesium sulfate, filtered and concentrated in vacuo to afford 1.30 g (79%) of the product as a deep red oil, which required no further purification.

LC-MS (Method 3): Rₜ=0.915 min. MS (Mass method 1): m/z=234 (M+H)⁺

### Intermediate 130

### Ethyl 3-(hydroxyimino)-2-[2-(2-methylphenyl)hydrazinylidene]propanoate

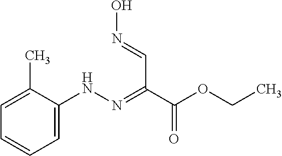 [SMILES: CCOC(=O)C(/C=N/O)=N/Nc1ccccc1C]

1.30 g (5.55 mmol) of ethyl 2-[2-(2-methylphenyl)hydrazinylidene]-3-oxopropanoate, 0.46 g (6.66 mmol) of hydroxylamine hydrochloride and 1.36 g (13.90 mmol) of potassium acetate were mixed in 30 mL of ethanol and heated to 78° C. for 30 min. The reaction was cooled to room temperature and 20 mL of water were added. The mixture was extracted with ethyl acetate and the combined organic extracts were dried over magnesium sulfate, filtered and concentrated to give 0.89 g (64%) of the product as a thick red oil. The compound was used in the next step without further purification.

LC-MS (Method 3): Rₜ=0.852 min. MS (Mass method 1): m/z=250 (M+H)⁺

### Intermediate 131

### Ethyl 2-(2-methylphenyl)-2H-1,2,3-triazole-4-carboxylate

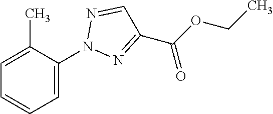 [SMILES: CCOC(=O)c1cnn(-c2ccccc2C)n1]

0.89 g (3.57 mmol) of ethyl 3-(hydroxyimino)-2-[2-(2-methylphenyl)hydrazinylidene propanoate were dissolved in 20 mL of acetic anhydride and the reaction was heated at 140° C. for 1 hour. The mixture was then cooled down to room temperature and 10 mL of water was added. The dark solution was extracted with dichloromethane and the combined organic layers were dried over magnesium sulfate, filtered and concentrated. The resulting residue was purified by flash chromatography on silica gel eluting with heptane/dichloromethane mixtures to afford 0.25 g (30%) of the product as a yellow oil.

¹H-NMR (300 MHz, DMSO-d₆): δ [ppm]=1.33 (t, 3H), 2.26 (s, 3H), 4.38 (q, 2H), 7.39-7.53 (m, 3H), 7.59 (d, 1H), 8.58 (s, 1H).

LC-MS (Method 3): Rₜ=0.875 min. MS (Mass method 1): m/z=232 (M+H)⁺

### Intermediate 132

### 2-(2-methylphenyl)-2H-1,2,3-triazole-4-carboxylic acid

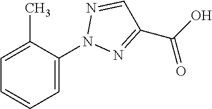

54 mg (1.30 mmol) of lithium hydroxide monohydrate were added to a solution of 250 mg (1.08 mmol) of ethyl 2-(2-methylphenyl)-2H-1,2,3-triazole-4-carboxylate in 4 mL of tetrahydrofuran aid 2 mL of water and the reaction was allowed to stir at room temperature for 2 hours. The pH was adjusted to 4-5 by addition of 2N hydrochloric acid(aq.) and the solvent was totally removed to yield 220 mg (99%) of the product as a yellow powder. The compound was used in the next step without further purification.

¹H-NMR (300 MHz, DMSO-d₆): δ [ppm]=2.28 (s, 3H), 7.34-7.48 (m, 3H), 7.55 (d, 1H), 8.18 (s, 1H)

LC-MS (Method 3): Rₜ=0.589 min. MS (Mass method 1): m/z=204 (M+H)⁺

### Intermediate 133

### Ethyl 2-[2-(4-methylphenyl)hydrazinylidene]-3-oxopropanoate

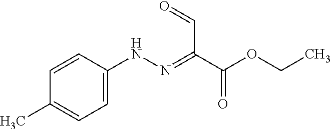 [SMILES: CCOC(=O)/C(C=O)=N/Nc1ccc(C)cc1]

0.48 g (6.98 mmol) of sodium nitrite in 10 mL of water were added dropwise to a cold solution of 0.75 mL (7.00 mmol) of 4-methylaniline in 3 mL of concentrated hydrochloric acid and 10 mL of water at 0° C. and the mixture was stirred for 5 min under the same conditions. This solution was added drop wise to a cold solution of 1.00 g (6.98 mmol) of (2E)-3-(dimethylamino)prop-2-enoate and 1.03 g (10.50 mmol) of potassium acetate in 15 mL of ethanol and the reaction was stirred at room temperature for 14 hours. The mixture was then diluted with ethyl acetate and the dark red solution was washed with water and brine. The aqueous phase was dried over magnesium sulfate, filtered and concentrated in vacuo to afford 1.19 g (73%) of the product as a deep red oil, which required no further purification.

LC-MS (Method 3): Rₜ=0.903 min. MS (Mass method 1): m/z=234 (M+H)⁺

### Intermediate 134

### Ethyl 3-(hydroxyimino)-2-[2-(4-methylphenyl)hydrazinylidene]propanoate

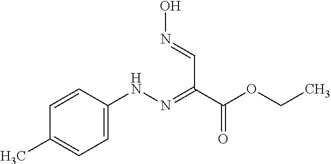 [SMILES: CCOC(=O)C(/C=N/O)=N/Nc1ccc(C)cc1]

1.19 g (5.08 mmol) of ethyl 2-[2-(4-methylphenyl)hydrazinylidene]-3-oxopropanoate, 0.42 g (6.10 mmol) of hydroxylamine hydrochloride and 1.25 g (12.70 mmol) of potassium acetate were mixed in 30 mL of ethanol and heated to 78° C. for 30 min. The reaction was cooled to room temperature and 20 mL of water were added. The mixture was extracted with ethyl acetate and the combined organic extracts were dried over magnesium sulfate, filtered and concentrated to give 1.19 g (94%) of the product as a thick red oil. The compound was used in the next step without further purification.

LC-MS (Method 3): Rₜ=0.858 min. MS (Mass method 1): m/z=250 (M+H)⁺

### Intermediate 135

### Ethyl 2-(4-methylphenyl)-2H-1,2,3-triazole-4-carboxylate

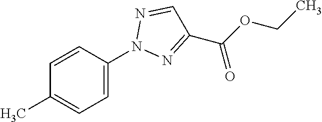 [SMILES: CCOC(=O)c1cnn(-c2ccc(C)cc2)n1]

1.19 g (4.77 mmol) of ethyl 3-(hydroxyimino)-2-[2-(4-methylphenyl)hydrazinylidene propanoate were dissolved in 20 mL of acetic anhydride and the reaction was heated at 140° C. for 1 hour. The mixture was then cooled down to room temperature and 10 mL of water was added. The dark solution was extracted with dichloromethane and the combined organic layers were dried over magnesium sulfate, filtered and concentrated. The resulting residue was purified by flash chromatography on silica gel eluting with heptane/dichloromethane mixtures to afford 0.25 g (22%) of the product as a yellow solid.

¹H-NMR (300 MHz, DMSO-d₆): δ [ppm]=1.34 (t, 3H), 2.38 (s, 3H), 4.38 (q, 2H), 7.41 (d, 2H), 7.95 (d, 2H), 8.56 (s, 1H).

LC-MS (Method 3): Rₜ=0.955 min. MS (Mass method 1): m/z=232 (M+H)⁺

### Intermediate 136

### 2-(4-methylphenyl)-2H-1,2,3-triazole-4-carboxylic acid

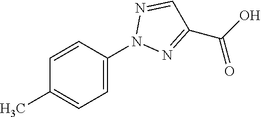

46 mg (1.09 mmol) of lithium hydroxide monohydrate were added to a solution of 210 mg (0.91 mmol) of ethyl 2-(4-methylphenyl)-2H-1,2,3-triazole-4-carboxylate in 4 mL of tetrahydrofuran aid 2 mL of water and the reaction was allowed to stir at room temperature for 2 hours. The pH was adjusted to 4-5 by addition of 2N hydrochloric acid(aq.) and the solvent was totally removed to yield 180 mg (97%) of the product as a yellow powder. The compound was used in the next step without further purification.

¹H-NMR (300 MHz, DMSO-d₆): δ [ppm]=2.37 (s, 3H), 7.37 (d, 2H), 7.90 (d, 2H), 8.13 (s, 1H).

LC-MS (Method 3): Rₜ=0.670 min. MS (Mass method 1): m/z=204 (M+H)⁺

### Intermediate 137

### Ethyl 2-[2-(4-cyanophenyl)hydrazinylidene]-3-oxopropanoate

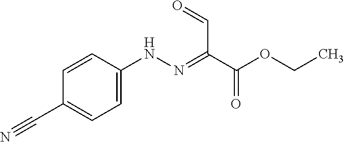 [SMILES: CCOC(=O)/C(C=O)=N/Nc1ccc(C#N)cc1]

0.48 g (6.98 mmol) of sodium nitrite in 10 mL of water were added dropwise to a cold solution of 0.82 g (7.00 mmol) of 4-aminobenzonitrile in 3 mL of concentrated hydrochloric acid and 10 mL of water at 0° C. and the mixture was stirred for 5 min under the same conditions. This solution was added drop wise to a cold solution of 1.00 g (6.98 mmol) of (2E)-3-(dimethylamino)prop-2-enoate and 1.03 g (10.50 mmol) of potassium acetate in 15 mL of ethanol and the reaction was stirred at room temperature for 14 hours. The mixture was then diluted with ethyl acetate and the dark red solution was washed with water and brine. The aqueous phase was dried over magnesium sulfate, filtered and concentrated in vacuo to afford 1.70 g (99%) of the product as a deep red orange oil, which required no further purification.

LC-MS (Method 3): Rₜ=0.745 min. MS (Mass method 1): m/z=246 (M+H)⁺

### Intermediate 138

### Ethyl 2-[2-(4-cyanophenyl)hydrazinylidene]-3-(hydroxyimino)propanoate

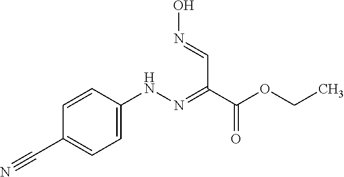 [SMILES: CCOC(=O)C(/C=N/O)=N/Nc1ccc(C#N)cc1]

1.70 g (6.93 mmol) of ethyl 2-[2-(4-cyanophenyl)hydrazinylidene]-3-oxopropanoate, 0.58 g (8.32 mmol) of hydroxylamine hydrochloride and 1.70 g (17.30 mmol) of potassium acetate were mixed in 30 mL of ethanol and heated to 78° C. for 30 min. The reaction was cooled to room temperature and 20 mL of water were added. The mixture was extracted with ethyl acetate and the combined organic extracts were dried over magnesium sulfate, filtered and concentrated to give 1.63 g (90%) of the product as a red oil. The compound was used in the next step without further purification.

LC-MS (Method 3): Rₜ=0.755 min. MS (Mass method 1): m/z=260 (M+H)⁺

### Intermediate 139

### Ethyl 2-(4-cyanophenyl)-2H-1,2,3-triazole-4-carboxylate

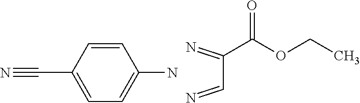

1.63 g (6.24 mmol) of ethyl 2-[2-(4-cyanophenyl)hydrazinylidene]-3-(hydroxyimino)propanoate were dissolved in 20 mL of acetic anhydride and the reaction was heated at 140° C. for 1 hour. The mixture was then cooled down to room temperature and 10 mL of water was added. The dark solution was extracted with dichloromethane and the combined organic layers were dried over magnesium sulfate, filtered and concentrated. The resulting residue was purified by flash chromatography on silica gel eluting with heptane/dichloromethane mixtures to afford 0.55 g (36%) of the product as a yellow solid.

¹H-NMR (300 MHz, DMSO-d₆): δ [ppm]=1.36 (t, 3H), 4.40 (q, 2H), 8.10 (d, 2H), 8.26 (d, 2H), 8.70 (s, 1H).

LC-MS (Method 3): Rₜ=0.808 min. MS (Mass method 1): m/z=243 (M+H)⁺

### Intermediate 140

### 2-(4-cyanophenyl)-2H-1,2,3-triazole-4-carboxylic acid

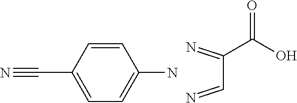

113 mg (2.70 mmol) of lithium hydroxide monohydrate were added to a solution of 545 mg (2.25 mmol) of ethyl 2-(4-cyanophenyl)-2H-1,2,3-triazole-4-carboxylate in 6 mL of tetrahydrofuran and 3 mL of water and the reaction was allowed to stir at room temperature for 2 hours. The pH was adjusted to 4-5 by addition of 2N hydrochloric acid(aq.) and the solvent was totally removed to yield 480 mg (99%) of the product as a yellow powder. The compound was used in the next step without further purification.

¹H-NMR (300 MHz, DMSO-d₆): δ [ppm]=8.10 (d, 2H), 8.25 (d, 2H), 8.61 (s, 1H).

LC-MS (Method 3): Rₜ=0.513 min. MS (Mass method 1): m/z=215 (M+H)⁺

### Intermediate 141

### Ethyl 2-[2-(3-cyanophenyl)hydrazinylidene]-3-oxopropanoate

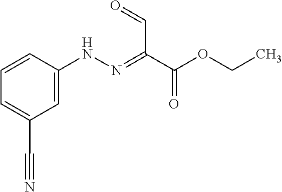 [SMILES: CCOC(=O)/C(C=O)=N/Nc1cccc(C#N)c1]

0.48 g (6.98 mmol) of sodium nitrite in 10 mL of water was added drop wise to solution of 0.82 g (6.98 mmol) of 3-aminobenzonitrile in 3 mL of concentrated hydrochloric acid and 5 mL of water at 0° C. and the mixture was stirred for 10 min under the same conditions. After that time, the resulting solution was added drop wise to a solution of 1.00 g (6.98 mmol) of ethyl (2E)-3-(dimethylamino)prop-2-enoate and 1.03 g (10.50 mmol) of potassium acetate in 15 mL of ethanol at 0° C. and then mixture was further stirred at room temperature for 12 hours. The ethanolic fraction of the solvent was removed and the resulting residue was diluted with ethyl acetate and washed with water. The organic layer was dried over magnesium sulfate, filtered and concentrated giving 1.69 g (98%) of the crude product as a deep orange thick oil. The compound was used as such in the next step.

LC-MS (Method 3): Rₜ=0.752 min. MS (Mass method 1): m/z=246 (M+H)⁺

### Intermediate 142

### Ethyl 2-[2-(3-cyanophenyl)hydrazinylidene]-3-(hydroxyimino)propanoate

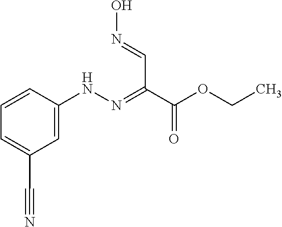 [SMILES: CCOC(=O)C(/C=N/O)=N/Nc1cccc(C#N)c1]

1.69 g (6.89 mmol) of ethyl 2-[2-(3-cyanophenyl)hydrazinylidene]-3-oxopropanoate, 0.58 g (8.27 mmol) of hydroxylamine hydrochloride and 1.69 g (17.20 mmol) potassium acetate were mixed in 30 mL of ethanol and the mixture was heated up to 78° C. for 1 hour. The resulting red suspension was cooled down to room temperature and 20 mL of water was added. The reaction was extracted with ethyl acetate and the combined organic layers were dried over magnesium sulfate, filtered and concentrated to give 1.60 g (89%) of the crude product as a thick red oil. The compound was used as such in the next step.

LC-MS (Method 3): Rₜ=0.765 min. MS (Mass method 1): m/z=261 (M+H)⁺

### Intermediate 143

### Ethyl 2-(3-cyanophenyl)-2H-1,2,3-triazole-4-carboxylate

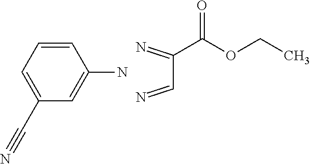

1.60 g (6.15 mmol) of ethyl 2-[2-(3-cyanophenyl)hydrazinylidene]-3-(hydroxyimino) propanoate were dissolved in 20 mL of acetic anhydride and the mixture was stirred at 140° C. for 1 hour. Upon cooling, 20 mL of water were added and the reaction was extracted with dichloromethane. The combined organic layers were dried over magnesium sulfate, filtered and concentrated to give a residue which was further purified by flash chromatography on silica gel using heptane/dichloromethane mixtures to afford 0.68 g (46%) of the product as a yellow solid.

¹H-NMR (300 MHz, DMSO-d₆): δ [ppm]=1.35 (t, 3H), 4.40 (q, 2H), 7.83 (t, 1H), 8.00 (d, 1H), 8.39 (d, 1H), 8.45 (s, 1H), 8.67 (s, 1H).

LC-MS (Method 3): Rₜ=0.809 min. MS (Mass method 1): m/z=243 (M+H)⁺

### Intermediate 144

### 2-(3-cyanophenyl)-2H-1,2,3-triazole-4-carboxylic acid

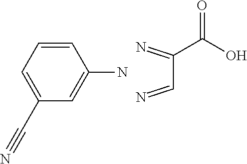

141 mg (3.35 mmol) of lithium hydroxide mono hydrate was added to a solution of 677 mg (2.79 mmol) of ethyl 2-(3-cyanophenyl)-2H-1,2,3-triazole-4-carboxylate in 6 mL of tetrahydrofuran and 3 mL of water and the mixture was stirred at room temperature for 2 hours. After that time, pH was set to 3-4 by addition of 2N hydrochloric acid(aq.) and the solvent was totally removed by rotary evaporation. 600 mg (99%) of the crude product was then isolated as a light red powder. The compound was used as such in the next synthetic step.

¹H-NMR (300 MHz, DMSO-d₆): δ [ppm]=7.82 (t, 1H), 7.97 (d, 1H), 8.37 (d, 1H), 8.42 (s, 1H), 8.57 (sm 1H).

LC-MS (Method 3): Rₜ=0.515 min. MS (Mass method 1): m/z=215 (M+H)⁺

### Intermediate 145

### Ethyl 2-[2-(3-methylphenyl)hydrazinylidene]-3-oxopropanoate

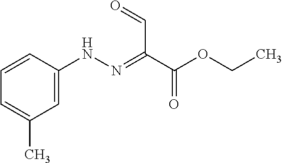 [SMILES: CCOC(=O)/C(C=O)=N/Nc1cccc(C)c1]

0.48 g (6.98 mmol) of sodium nitrite in 10 mL of water was added drop wise to solution of 0.75 mL (7.00 mmol) of 3-methylaniline in 3 mL of concentrated hydrochloric acid and 5 mL of water at 0° C. and the mixture was stirred for 10 min under the same conditions. After that time, the resulting solution was added drop wise to a solution of 1.00 g (6.98 mmol) of ethyl (2E)-3-(dimethylamino)prop-2-enoate and 1.03 g (10.50 mmol) of potassium acetate in 15 mL of ethanol at 0° C. and then mixture was further stirred at room temperature for 12 hours. The ethanolic fraction of the solvent was removed and the resulting residue was diluted with ethyl acetate and washed with water. The organic layer was dried over magnesium sulfate, filtered and concentrated giving 1.45 g (88%) of the crude product as a deep red thick oil. The compound was used as such in the next step.

LC-MS (Method 3): Rₜ=0.907 min. MS (Mass method 1): m/z=235 (M+H)⁺

### Intermediate 146

### Ethyl-3-(hydroxyimino)-2-[2-(3-methylphenyl)hydrazinylidene]propanoate

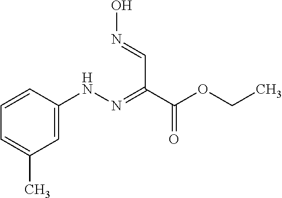 [SMILES: CCOC(=O)C(/C=N/O)=N/Nc1cccc(C)c1]

1.45 g (6.19 mmol) of ethyl 2-[2-(3-methylphenyl)hydrazinylidene]-3-oxopropanoate, 0.52 g (7.43 mmol) of hydroxylamine hydrochloride and 1.52 g (15.50 mmol) potassium acetate were mixed in 30 mL of ethanol and the mixture was heated up to 78° C. for 1 hour. The resulting red suspension was cooled down to room temperature and 20 mL of water was added. The reaction was extracted with ethyl acetate and the combined organic layers were dried over magnesium sulfate, filtered and concentrated to give 1.17 g (76%) of the crude product as a thick red oil. The compound was used as such in the next step.

LC-MS (Method 3): Rₜ=0.855 min. MS (Mass method 1): m/z=250 (M+H)⁺

### Intermediate 147

### Ethyl 2-(3-methylphenyl)-2H-1,2,3-triazole-4-carboxylate

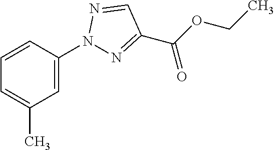 [SMILES: CCOC(=O)c1cnn(-c2cccc(C)c2)n1]

1.17 g (4.69 mmol) of ethyl 3-(hydroxyimino)-2-[2-(3-methylphenyl) hydrazinylidene]propanoate were dissolved in 20 mL of acetic anhydride and the mixture was stirred at 140° C. for 1 hour. Upon cooling, 20 mL of water were added and the reaction was extracted with dichloromethane. The combined organic layers were dried over magnesium sulfate, filtered and concentrated to give a residue which was further purified by flash chromatography on silica gel using heptane/dichloromethane mixtures to afford 0.30 g (28%) of the product as a yellow oil.

¹H-NMR (300 MHz, DMSO-d₆): δ [ppm]=1.34 (t, 3H), 2.43 (s, 3H), 4.38 (q, 2H), 7.33 (d, 1H), 7.48 (t, 1H), 7.82-7.91 (m, 2H), 8.57 (s, 1H).

LC-MS (Method 3): Rₜ=0.959 min. MS (Mass method 1): m/z=232 (M+H)⁺

### Intermediate 148

### 2-(3-methylphenyl)-2H-1,2,3-triazole-4-carboxylic acid

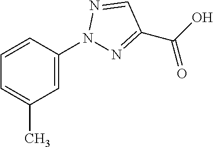

65 mg (1.56 mmol) of lithium hydroxide monohydrate was added to a solution of 300 mg (1.30 mmol) of ethyl 2-(3-methylphenyl)-2H-1,2,3-triazole-4-carboxylate in 4 mL of tetrahydrofuran aid 2 mL of water and the mixture was stirred at room temperature for 2 hours. After that time, pH was set to 3-4 by addition of 2N hydrochloric acid(aq.) and the solvent was totally removed by rotary evaporation. 260 mg (98%) of the crude product was then isolated as a light yellow powder. The compound was used as such in the next synthetic step.

¹H-NMR (300 MHz, DMSO-d₆): δ [ppm]=2.40 (s, 3H), 7.21 (d, 1H), 7.42 (t, 1H), 7.78 (d, 1H), 7.83 (s, 1H), 7.99 (s, 1H).

LC-MS (Method 3): Rₜ=0.671 min. MS (Mass method 1): m/z=205 (M+H)⁺

### Intermediate 149

### 2-(pyridin-2-yl)-2H-1,2,3-triazole-4-carboxylic acid

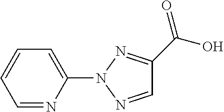 [SMILES: O=C(O)c1cnn(-c2ccccn2)n1]

1.00 g (7.87 mmol) of methyl-2H-1,2,3-triazole-4-carboxylate, 0.76 mL (7.20 mmol) of 2-iodopyridine, 0.06 g (0.72 mmol) of copper(II) oxide, 0.76 g (2.15 mmol) of iron(III) acetylacetonate and 4.66 g (14.30 mmol) of cesium carbonate were mixed in 10 mL of N,N-dimethylformamide and the resulting suspension was heated at 100° C. for 12 hours. The reaction mixture was diluted with dichloromethane and extracted with water. The combined aqueous extracts were filtered through a pad of celite and concentrated under vacuum to afford a dark crude, which was further purified by flash column chromatography on silica gel eluting with dichloromethane/methanol mixtures to give 0.30 g (20%) of the product as a yellow powder.

¹H-NMR (300 MHz, DMSO-d₆): δ [ppm]=7.60 (t, 1H), 8.05-8.22 (m, 2H), 8.56 (s, 1H), 8.64 (d, 1H)

LC-MS (Method 3): Rₜ=0.296 min. MS (Mass method 1): m/z=191 (M+H)⁺

### Intermediate 150

### 2-(5-fluoropyridin-2-yl)-2H-1,2,3-triazole-4-carboxylic acid

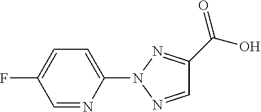 [SMILES: O=C(O)c1cnn(-c2ccc(F)cn2)n1]

278 mg (2.19 mmol) of methyl-2H-1,2,3-triazole-4-carboxylate, 350 mg (1.99 mmol) of 2-bromo-5-fluoropyridine, 16 mg (0.20 mmol) of copper(II) oxide, 211 mg (0.60 mmol) of iron(III) acetylacetonate and 1300 mg (3.98 mmol) of cesium carbonate were mixed in 5 mL of N,N-dimethylformamide and the resulting suspension was heated at 100° C. for 12 hours. The reaction mixture was diluted with dichloromethane and extracted with water. The combined aqueous extracts were filtered through a pad of celite and concentrated under vacuum to afford a dark crude, which was further purified by flash column chromatography on silica gel eluting with dichloromethane/methanol/acetic acid mixtures to give 400 mg (88%) of the product as a yellow powder.

LC-MS (Method 3): Rₜ=0.322 min. MS (Mass method 1): m/z=209 (M+H)⁺

### Intermediate 151

### Methyl 2-(pyrazin-2-yl)-2H-1,2,3-triazole-4-carboxylate

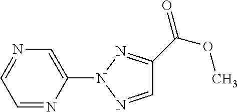

250 mg (1.96 mmol) of methyl-2H-1,2,3-triazole-4-carboxylate, 0.32 mL (3.94 mmol) of 2-fluoropyrazine and 730 mg (5.31 mmol) potassium carbonate were dissolved in 6 mL of N,N-dimethylformamide. To this mixture, 49 mg (0.29 mmol) of potassium iodide and 52 mg (0.20 mmol) of 18-crown-6 were added and the reaction was stirred at 135° C. for 23 hours. After completion of the reaction, the suspension was concentrated under reduced pressure and the residue was dissolved in ethyl acetate and washed with water. The organic layer was dried over magnesium sulfate, filtered, concentrated and chromatographed in heptane/ethyl acetate mixtures to afford 70 mg (17%) of the product as a thick yellow oil, which solidified upon standing.

¹H-NMR (300 MHz, DMSO-d₆): δ [ppm]=3.93 (s, 3H), 8.71-8.78 (m, 2H), 8.85 (s, 1H), 9.37 (s, 1H)

LC-MS (Method 3): Rₜ=0.401 min. MS (Mass method 1): m/z=206 (M+H)⁺

### Intermediate 152

### 2-(pyrazin-2-yl)-2H-1,2,3-triazole-4-carboxylic acid

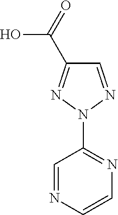 [SMILES: O=C(O)c1cnn(-c2cnccn2)n1]

15 mg (0.35 mmol) of lithium hydroxide were added to a solution of 60 mg (0.29 mmol) of methyl 2-(pyrazin-2-yl)-2H-1,2,3-triazole-4-carboxylate in 2 mL of tetrahydrofuran and 1 mL of water and the mixture was stirred at room temperature for 5 hours. Then, pH was set to 4-5 by addition of 2N hydrochloric acid and the solvent was removed in vacuo to give 60 mg (98%) of the product as a light yellow powder. The compound was used in the next step without further purification.

LC-MS (Method 3): Rₜ=0.159 min. MS (Mass method 1): m/z=192 (M+H)⁺

### Intermediate 153

### 1-(cyclopropylmethyl)-1H-pyrazole-4-carbaldehyde

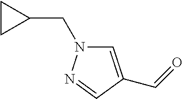 [SMILES: O=Cc1cnn(CC2CC2)c1]

0.5 g (5.20 mmol) of pyrazole-4-carbaldehyde were added to a suspension of 0.23 g (5.72 mmol) 60% sodium hydride (as a dispersion in mineral oil) in 15 mL of N,N-dimethylformamide at 0° C. and the mixture was stirred for 30 min. After that time, a solution of 0.56 mL (5.72 mmol) of cyclopropylmethyl bromide in 5 mL of N,N-dimethylformamide was added dropwise and the mixture was stirred at room temperature for 18 hours. The reaction was diluted with ethyl acetate and extracted with water. The organic layer was dried over magnesium sulfate, filtered and concentrated under vacuum affording a crude which was purified by flash column chromatography on silica gel eluting with heptane/ethyl acetate mixtures to give 0.66 g (84%) of the product as a yellow oil.

¹H-NMR (300 MHz, DMSO-d₆): δ [ppm]=0.34-0.42 (m, 2H), 0.50-0.59 (m, 2H), 1.19-1.34 (m, 1H), 4.03 (d, 2H), 7.98 (s, 1H), 8.49 (s, 1H), 9.79 (s, 1H).

LC-MS (Method 3): Rₜ=0.432 min. MS (Mass method 1): m/z=151 (M+H)⁺

### Intermediate 154

### N-{[1-(cyclopropylmethyl)-1H-pyrazol-4-yl]methylidene}hydroxylamine

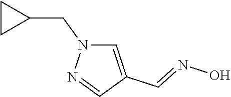 [SMILES: O/N=C/c1cnn(CC2CC2)c1]

0.53 mL (8.70 mmol) of 50% hydroxylamine in water were dissolved in 6 mL of ethanol. Then, a solution of 0.65 g (4.63 mmol) of 1-(cyclopropylmethyl)-1H-pyrazole-4-carbaldehyde in 6 mL of ethanol was added and the mixture was stirred for 30 min at room temperature. The reaction was concentrated and the resulting residue was dissolved in toluene in order to remove the remains of water azeotropically. 0.71 g (98%) of the crude product were isolated as a tan oil, which required no further purification.

¹H-NMR (300 MHz, DMSO-d₆): δ [ppm]=0.34-0.40 (m, 2H), 0.47-0.57 (d, 2H), 1.16-1.31 (m, 1H), 4.00 (d, 2H), 7.35 (s, 1H), 7.80 (s, 1H), 8.28 (s, 1H), 11.20 (bp, 1H).

LC-MS (Method 3): Rₜ=0.421 min. MS (Mass method 1): m/z=166 (M+H)⁺

### Intermediate 155

### Ethyl 3-[1-(cyclopropylmethyl)-1H-pyrazol-4-yl]-1,2-oxazole-5-carboxylate

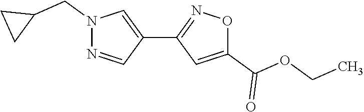 [SMILES: CCOC(=O)c1cc(-c2cnn(CC3CC3)c2)no1]

0.56 g (4.18 mmol) of N-chlorosuccinimide were added to a solution of 0.66 g (3.98 mmol) of N-{[1-(cyclopropylmethyl)-1H-pyrazol-4-yl]methylidene}hydroxylamine in 25 mL of dichloromethane at room temperature and the mixture was stirred for 5 min. Then, 1.61 mL (15.93 mmol) of ethyl propiolate and 0.83 mL (5.97 mmol) of triethylamine were sequentially added drop wise and the reaction was stirred at room temperature for 18 hours. The reaction mixture was washed with water and brine and the organic layer was dried over magnesium sulfate, filtered, concentrated and chromatographed by flash column chromatography on silica gel eluting with heptane/ethyl acetate mixtures to give 0.53 g (51%) of the product as an orange oil

LC-MS (Method 3): Rₜ=0.815 min. MS (Mass method 1): m/z=262 (M+H)⁺

### Intermediate 156

### 3-[1-(cyclopropylmethyl)-1H-pyrazol-4-yl]-1,2-oxazole-5-carboxylic acid

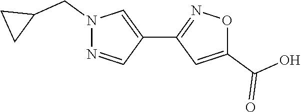 [SMILES: O=C(O)c1cc(-c2cnn(CC3CC3)c2)no1]

104 mg (2.47 mmol) of lithium hydroxide monohydrate were added to a solution of 430 mg (1.65 mmol) of ethyl 3-[1-(cyclopropylmethyl)-1H-pyrazol-4-yl]-1,2-oxazole-5-carboxylate in 6 mL of tetrahydrofuran and 4 mL of water and the mixture was stirred at room temperature for 1 hour. The pH was then adjusted to 4-5 by addition of 2N hydrochloric acid(aq.) and the reaction was concentrated by rotary evaporation to yield 380 mg (99%) of the crude product as an orange oil. The isolated compound was used as such in the next synthetic step.

LC-MS (Method 3): Rₜ=0.431 min. MS (Mass method 1): m/z=234 (M+H)⁺

### Intermediate 157

### Methyl N-benzoyl-beta-alaninate

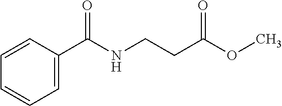

1.50 mL (13.00 mmol) of benzoyl chloride were carefully added to a solution of 1.98 g (14.20 mmol) of beta-alanine methyl ester hydrochloride and 4.50 mL (32.00 mmol) of triethylamine in 30 mL of dichloromethane and the mixture was stirred at room temperature for 12 hours. The reaction was quenched with water and the organic layer was separated, dried over magnesium sulfate, filtered, concentrated and chromatographed in heptane/ethyl acetate mixtures to afford 2.50 g (93%) of the product as a colourless thick oil which crystallised with time.

¹H-NMR (300 MHz, DMSO-d₆): δ [ppm]=2.59 (t, 2H), 3.49 (q, 2H), 3.60 (s, 3H), 7.40-7.56 (m, 3H), 7.82 (d, 2H), 8.54 (t, 1H).

LC-MS (Method 3): Rₜ=0.515 min. MS (Mass method 1): m/z=208 (M+H)⁺

### Intermediate 158

### Methyl 2-phenyl-1,3-oxazole-5-carboxylate

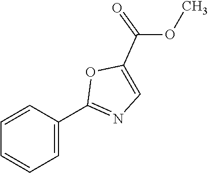

1.50 g (7.24 mmol) of methyl N-benzoyl-beta-alaninate, 3.86 g (21.70 mmol) of N-bromosuccinimide and 2.00 g (14.50 mmol) of potassium carbonate were dissolved in 30 mL of 1,2-dichloroethane and the reaction was stirred at 100° C. for 48 hours. Upon cooling, the mixture was quenched with sodium thiosulfate(sat.) and the reaction was extracted with diethyl ether. The combined organic layers were dried over magnesium sulfate, filtered, concentrated and chromatographed in heptane/ethyl acetate mixtures to afford 0.16 g (11%) of the product as a white powder.

¹H-NMR (300 MHz, DMSO-d₆): δ [ppm]=3.88 (s, 3H), 7.54-7.64 (m, 2H), 8.06 (d, 2H), 8.13 (s, 1H)

LC-MS (Method 3): Rₜ=0.838 min. MS (Mass method 1): m/z=204 (M+H)⁺

### Intermediate 159

### 2-Phenyl-1,3-oxazole-5-carboxylic acid

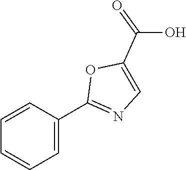 [SMILES: O=C(O)c1cnc(-c2ccccc2)o1]

40 mg (0.94 mmol) of lithium hydroxide monohydrate were added to a solution of 160 mg (0.78 mmol) of methyl 2-phenyl-1,3-oxazole-5-carboxylate in 4 mL of tetrahydrofuran and 2 mL of water and the mixture was stirred at room temperature for 10 hours. The pH was then adjusted to 4-5 by addition of 2N hydrochloric acid(aq.) and the mixture was concentrated by rotary evaporation giving 150 mg (100%) of the crude product as a fluffy white solid which was used as such in the next synthetic step.

¹H-NMR (300 MHz, DMSO-d₆): δ [ppm]=7.53-7.61 (m, 3H), 7.80 (s, 1H), 7.99-8.06 (m, 2H).

LC-MS (Method 3): Rₜ=0.566 min. MS (Mass method 1): m/z=190 (M+H)⁺

### Intermediate 160

### Ethyl (2E)-2-[(4-methoxyphenyl)hydrazono]-3-oxopropanoate

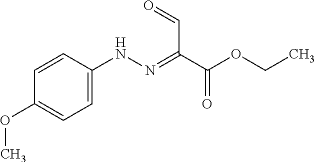 [SMILES: CCOC(=O)/C(C=O)=N/Nc1ccc(OC)cc1]

4-Methoxyaniline (1.00 g, 8.12 mmol) was dissolved in 10 ml of water and 2 ml of concentrated hydrochloric acid. The mixture was cooled to 0° C. and a solution of sodium nitrite (560 mg, 8.12 mmol) in 5 ml of water was added dropwise. After stirring for 5 minutes this solution was added dropwise to a solution of ethyl-(2E)-3-(dimethylamino)acrylate (1.3 ml, 8.9 mmol) and potassium acetate (1.20 g, 12.2 mmol) in 15 ml of ethanol at 0° C. The mixture was stirred at room temperature for one hour before being diluted with ethyl acetate and washed twice with water and once with brine. The organic layer was dried over sodium sulfate, filtered and evaporated. 2.03 g (67% yield, 67% purity) of the title compound were obtained.

LC-MS (Method 7): Rₜ=1.79 min; MS (ESIpos): m/z=251 [M+H]⁺

### Intermediate 161

### Ethyl (2E,3E)-3-(hydroxyimino)-2-[(4-methoxyphenyl)hydrazono]propanoate

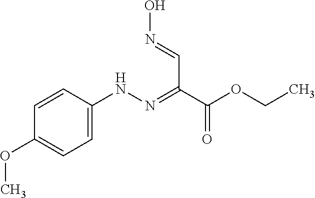 [SMILES: CCOC(=O)C(/C=N/O)=N/Nc1ccc(OC)cc1]

Ethyl (2E)-2-[(4-methoxyphenyl)hydrazono]-3-oxopropanoate (2.03 g, 67% purity, 5.44 mmol), hydroxylamine hydrochloride (454 mg, 6.53 mmol) and potassium acetate (1.33 g, 13.6 mmol) were dissolved in 20 ml of ethanol and the mixture was stirred for 30 minutes at 80° C. The reaction mixture was diluted with ethyl acetate and washed twice with water and once with brine. The organic layer was dried over sodium sulfate, filtered and evaporated. The residue was dried in vacuum. 1.87 g (46% purity, 60% yield) of the title compound were obtained.

LC-MS (Method 7): Rₜ=1.68 min; MS (ESIpos): m/z=266 [M+H]⁺

### Intermediate 162

### Ethyl 2-(4-methoxyphenyl)-2H-1,2,3-triazole-4-carboxylate

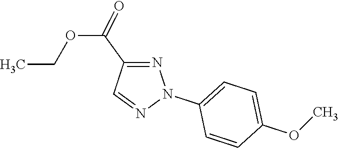 [SMILES: CCOC(=O)c1cnn(-c2ccc(OC)cc2)n1]

Ethyl (2E,3E)-3-(hydroxyimino)-2-[(4-methoxyphenyl)hydrazono]propanoate (1.87 g, 7.05 mmol) were stirred for one hour in 15 ml of acetic acid anhydride at 140° C. The reaction was then treated with water and extracted with dichlormethane. The organic layer was dried over sodium sulfate, filtered and evaporated. The residue was put on Isolute and purified by chromatography on silica gel (100 g Ultra Snap Cartridge Biotage®; Biotage-Isolera-One®, Cy/EE-gradient: 5% EE-40% EE; flow: 100 ml/min). Samples containing the desired product were united, the solvents were evaporated and the residue was dried in vacuum. 1.02 g (100% purity, 58% yield) of the title compound were obtained.

LC-MS (Method 7): Rₜ=1.91 min; MS (ESIpos): m/z=248 [M+H]⁺

### Intermediate 163

### 2-(4-methoxyphenyl)-2H-1,2,3-triazole-4-carboxylic acid

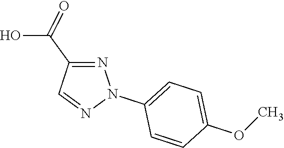 [SMILES: COc1ccc(-n2ncc(C(=O)O)n2)cc1]

Ethyl 2-(4-methoxyphenyl)-2H-1,2,3-triazole-4-carboxylate (1.02 g, 4.12 mmol) were dissolved in 10 ml of THF, treated with lithium hydroxide solution (4.1 ml, 1.0 M, 4.1 mmol) and the mixture was stirred over night at room temperature. Additional lithium hydroxide solution (2.0 ml, 1.0 M, 2.0 mmol) was added and the mixture was stirred for two more hours. The organic solvent was removed on a rotary evaporator, the aqueous residue was diluted with water and acidified with 1 N hydrochloric acid. The precipitate was filtered off, washed with water and dried in vacuum. 888 mg (99% purity, 97% yield) of the title compound were obtained.

LC-MS (Method 7): Rₜ=1.25 min; MS (ESIpos): m/z=220 [M+H]⁺

### Intermediate 164

### Ethyl (2E)-2-{[4-(methylsulfonyl)phenyl]hydrazono}-3-oxopropanoate

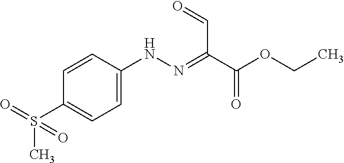 [SMILES: CCOC(=O)/C(C=O)=N/Nc1ccc(S(C)(=O)=O)cc1]

The reaction was carried out analogously to the preparation of ethyl (2E)-2-[(4-methoxyphenyl)hydrazono]-3-oxopropanoate. 1.94 g (75% purity, 83% yield) of the title compound were obtained.

LC-MS (Method 7): Rₜ=1.39 min; MS (ESIpos): m/z=299 [M+H]⁺

### Intermediate 165

### Ethyl (2E,3E)-3-(hydroxyimino)-2-{[4-(methylsulfonyl)phenyl]hydrazono}propanoate

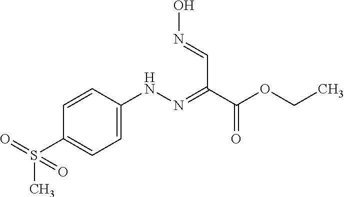 [SMILES: CCOC(=O)C(/C=N/O)=N/Nc1ccc(S(C)(=O)=O)cc1]

The reaction was carried out analogously to the preparation of ethyl (2 E,3E)-3-(hydroxyimino)-2-[(4-methoxyphenyl)hydrazono]propanoate. 1.53 g (100% purity, 100% yield) of the title compound were obtained.

LC-MS (Method 8): Rₜ=0.79 min; MS (ESIpos): m/z=314 [M+H]⁺

### Intermediate 166

### Ethyl 2-[4-(methylsulfonyl)phenyl]-2H-1,2,3-triazole-4-carboxylate

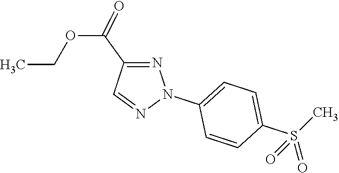 [SMILES: CCOC(=O)c1cnn(-c2ccc(S(C)(=O)=O)cc2)n1]

The reaction was carried out analogously to the preparation of ethyl 2-(4-methoxyphenyl)-2H-1,2,3-triazole-4-carboxylate. 989 mg (95% purity, 65% yield) of the title compound were obtained.

LC-MS (Method 7): Rₜ=1.52 min; MS (ESIpos): m/z=296 [M+H]⁺

### Intermediate 167

### 2-[4-(methylsulfonyl)phenyl]-2H-1,2,3-triazole-4-carboxylic acid

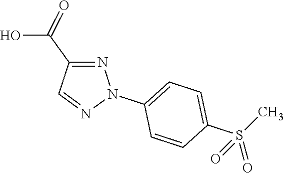 [SMILES: CS(=O)(=O)c1ccc(-n2ncc(C(=O)O)n2)cc1]

The reaction was carried out analogously to the preparation of 2-(4-methoxyphenyl)-2H-1,2,3-triazole-4-carboxylic acid. 576 mg (96% purity, 62% yield) of the title compound were obtained.

LC-MS (Method 7): Rₜ=0.83 min; MS (ESIneg): m/z=266 [M−H]⁻

### Intermediate 168

### Ethyl (2E)-2-[2-(2,3-difluorophenyl)hydrazinylidene]-3-oxopropanoate

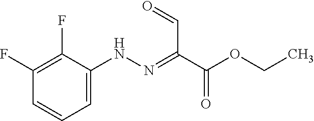 [SMILES: CCOC(=O)/C(C=O)=N/Nc1cccc(F)c1F]

The reaction was carried out analogously to the preparation of ethyl (2E)-2-[(4-methoxyphenyl)hydrazono]-3-oxopropanoate. 913 mg (88% purity, 81% yield) of the title compound were obtained.

LC-MS (Method 7): Rₜ=1.96 min; MS (ESIneg): m/z=255 [M−H]⁻

### Intermediate 169

### Ethyl (2E,3E)-2-[2-(2,3-difluorophenyl)hydrazinylidene]-3-(hydroxyimino)propanoate

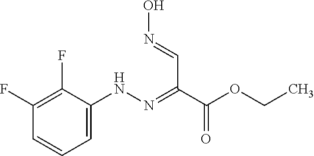 [SMILES: CCOC(=O)C(/C=N/O)=N/Nc1cccc(F)c1F]

The reaction was carried out analogously to the preparation of ethyl (2E,3E)-3-(hydroxyimino)-2-[(4-methoxyphenyl)hydrazono]propanoate. 869 mg (35% purity, 36% yield) of the title compound were obtained.

LC-MS (Method 7): Rₜ=1.87 min; MS (ESIpos): m/z=272 [M+H]⁺

### Intermediate 170

### Ethyl 2-(2,3-difluorophenyl)-2H-1,2,3-triazole-4-carboxylate

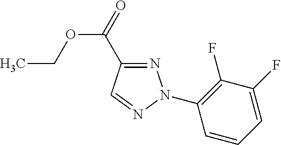 [SMILES: CCOC(=O)c1cnn(-c2cccc(F)c2F)n1]

The reaction was carried out analogously to the preparation of ethyl 2-(4-methoxyphenyl)-2H-1,2,3-triazole-4-carboxylate. 236 mg (92% purity, 75% yield) of the title compound were obtained.

LC-MS (Method 7): Rₜ=1.85 min; MS (ESIpos): m/z=254 [M+H]⁺

### Intermediate 171

### 2-(2,3-difluorophenyl)-2H-1,2,3-triazole-4-carboxylic acid

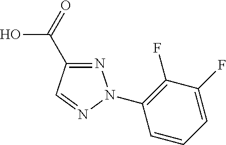 [SMILES: O=C(O)c1cnn(-c2cccc(F)c2F)n1]

The reaction was carried out analogously to the preparation of 2-(4-methoxyphenyl)-2H-1,2,3-triazole-4-carboxylic acid. 160 mg (100% purity, 83% yield) of the title compound were obtained.

LC-MS (Method 7): Rₜ=1.17 min; MS (ESIneg): m/z=224 [M−H]⁻

### Intermediate 172

### Ethyl (2E)-2-[2-(2,5-difluorophenyl)hydrazinylidene]-3-oxopropanoate

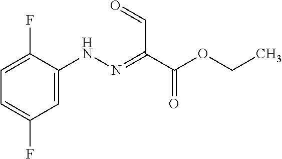 [SMILES: CCOC(=O)/C(C=O)=N/Nc1cc(F)ccc1F]

The reaction was carried out analogously to the preparation of ethyl (2E)-2-[(4-methoxyphenyl)hydrazono]-3-oxopropanoate. 947 mg (91% purity, 87% yield) of the title compound were obtained.

LC-MS (Method 7): Rₜ=1.93 min; MS (ESIpos): m/z=257 [M+H]⁺

### Intermediate 173

### Ethyl (2E,3E)-2-[2-(2,5-difluorophenyl)hydrazinylidene]-3-(hydroxyimino)propanoate

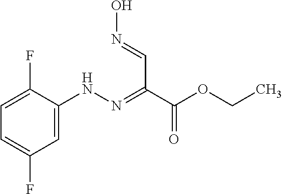 [SMILES: CCOC(=O)C(/C=N/O)=N/Nc1cc(F)ccc1F]

The reaction was carried out analogously to the preparation of ethyl (2E,3E)-3-(hydroxyimino)-2-[(4-methoxyphenyl)hydrazono]propanoate. 794 mg (77% purity, 67% yield) of the title compound were obtained.

LC-MS (Method 7): Rₜ=1.83 min; MS (ESIpos): m/z=272 [M+H]⁺

### Intermediate 174

### Ethyl 2-(2,5-difluorophenyl)-2H-1,2,3-triazole-4-carboxylate

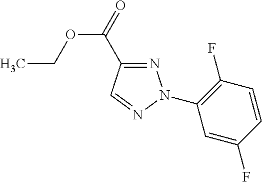 [SMILES: CCOC(=O)c1cnn(-c2cc(F)ccc2F)n1]

The reaction was carried out analogously to the preparation of ethyl 2-(4-methoxyphenyl)-2H-1,2,3-triazole-4-carboxylate. 411 mg (90% purity, 65% yield) of the title compound were obtained.

LC-MS (Method 7): Rₜ=1.82 min; MS (ESIpos): m/z=254 [M+H]⁺

### Intermediate 175

### 2-(2,5-difluorophenyl)-2H-1,2,3-triazole-4-carboxylic acid

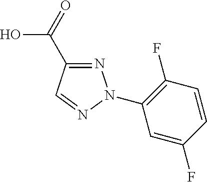 [SMILES: O=C(O)c1cnn(-c2cc(F)ccc2F)n1]

The reaction was carried out analogously to the preparation of 2-(4-methoxyphenyl)-2H-1,2,3-triazole-4-carboxylic acid. 70.0 mg (100% purity, 21% yield) of the title compound were obtained.

LC-MS (Method 8): Rₜ=0.64 min; MS (ESIneg): m/z=224 [M−H]⁻

### Intermediate 176

### Ethyl (2E)-2-[2-(2-fluorophenyl)hydrazinylidene]-3-oxopropanoate

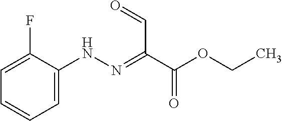 [SMILES: CCOC(=O)/C(C=O)=N/Nc1ccccc1F]

The reaction was carried out analogously to the preparation of ethyl (2E)-2-[(4-methoxyphenyl)hydrazono]-3-oxopropanoate. 1.01 g (84% purity, 79% yield) of the title compound were obtained.

LC-MS (Method 7): Rₜ=1.92 min; MS (ESIpos): m/z=239 [M+H]⁺

### Intermediate 177

### Ethyl (2E,3E)-2-[2-(2-fluorophenyl)hydrazinylidene]-3-(hydroxyimino)propanoate

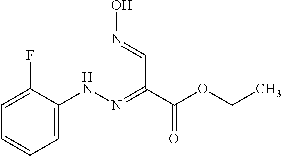 [SMILES: CCOC(=O)C(/C=N/O)=N/Nc1ccccc1F]

The reaction was carried out analogously to the preparation of ethyl (2E,3E)-3-(hydroxyimino)-2-[(4-methoxyphenyl)hydrazono]propanoate. 1.10 g (81% purity, 99% yield) of the title compound were obtained.

LC-MS (Method 7): Rₜ=1.79 min; MS (ESIpos): m/z=254 [M+H]⁺

### Intermediate 178

### Ethyl 2-(2-fluorophenyl)-2H-1,2,3-triazole-4-carboxylate

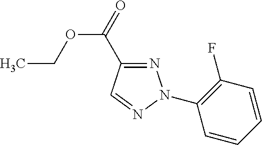 [SMILES: CCOC(=O)c1cnn(-c2ccccc2F)n1]

The reaction was carried out analogously to the preparation of ethyl 2-(4-methoxyphenyl)-2H-1,2,3-triazole-4-carboxylate. 594 mg (98% purity, 71% yield) of the title compound were obtained.

LC-MS (Method 7): Rₜ=1.76 min; MS (ESIpos): m/z=236 [M+H]⁺

### Intermediate 179

### 2-(2-fluorophenyl)-2H-1,2,3-triazole-4-carboxylic acid

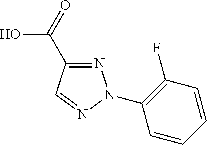 [SMILES: O=C(O)c1cnn(-c2ccccc2F)n1]

The reaction was carried out analogously to the preparation of 2-(4-methoxyphenyl)-2H-1,2,3-triazole-4-carboxylic acid. 383 mg (98% purity, 72% yield) of the title compound were obtained.

LC-MS (Method 7): Rₜ=1.06 min; MS (ESIpos): m/z=208 [M+H]⁺

### Intermediate 180

### Ethyl (2E)-2-[2-(3-chloro-4-fluorophenyl)hydrazinylidene]-3-oxopropanoate

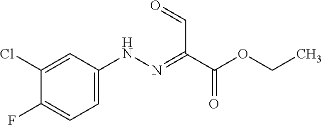 [SMILES: CCOC(=O)/C(C=O)=N/Nc1ccc(F)c(Cl)c1]

The reaction was carried out analogously to the preparation of ethyl (2E)-2-[(4-methoxyphenyl)hydrazono]-3-oxopropanoate. 883 mg (71% purity, 66% yield) of the title compound were obtained.

LC-MS (Method 7): Rₜ=2.01 min; MS (ESIpos): m/z=273 [M+H]⁺

### Intermediate 181

### Ethyl (2E,3E)-2-[2-(3-chloro-4-fluorophenyl)hydrazinylidene]-3-(hydroxyimino)propanoate

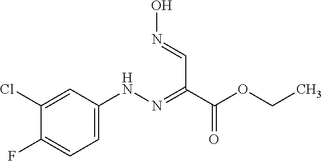 [SMILES: CCOC(=O)C(/C=N/O)=N/Nc1ccc(F)c(Cl)c1]

The reaction was carried out analogously to the preparation of ethyl (2E,3E)-3-(hydroxyimino)-2-[(4-methoxyphenyl)hydrazono]propanoate. 852 mg (67% purity, 87% yield) of the title compound were obtained.

LC-MS (Method 7): Rₜ=1.93 min; MS (ESIpos): m/z=288 [M+H]⁺

### Intermediate 182

### Ethyl 2-(3-chloro-4-fluorophenyl)-2H-1,2,3-triazole-4-carboxylate

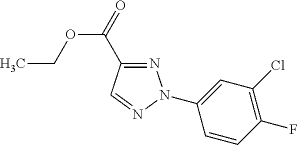 [SMILES: CCOC(=O)c1cnn(-c2ccc(F)c(Cl)c2)n1]

The reaction was carried out analogously to the preparation of ethyl 2-(4-methoxyphenyl)-2H-1,2,3-triazole-4-carboxylate. 287 mg (54% yield) of the title compound were obtained.

¹H-NMR (400 MHz, DMSO-d6) δ [ppm]: 1.330 (7.67), 1.348 (16.00), 1.366 (7.83), 4.367 (2.45), 4.385 (7.42), 4.403 (7.34), 4.421 (2.37), 7.662 (1.84), 7.684 (3.83), 7.707 (2.10), 8.063 (1.05), 8.070 (1.49), 8.080 (1.24), 8.086 (1.06), 8.093 (1.39), 8.103 (0.95), 8.221 (1.17), 8.227 (1.03), 8.236 (1.22), 8.243 (0.94), 8.643 (4.18).

### Intermediate 183

### 2-(3-chloro-4-fluorophenyl)-2H-1,2,3-triazole-4-carboxylic acid

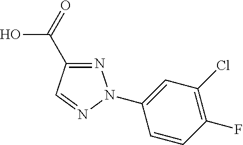 [SMILES: O=C(O)c1cnn(-c2ccc(F)c(Cl)c2)n1]

The reaction was carried out analogously to the preparation of 2-(4-methoxyphenyl)-2H-1,2,3-triazole-4-carboxylic acid. 116 mg (100% purity, 45% yield) of the title compound were obtained.

LC-MS (Method 7): Rₜ=1.48 min; MS (ESIpos): m/z=242 [M+H]⁺

### Intermediate 184

### Ethyl (2E)-2-[2-(4-chloro-3-fluorophenyl)hydrazinylidene]-3-oxopropanoate

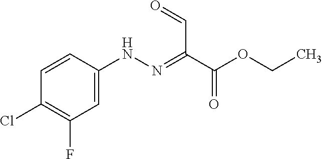 [SMILES: CCOC(=O)/C(C=O)=N/Nc1ccc(Cl)c(F)c1]

The reaction was carried out analogously to the preparation of ethyl (2E)-2-[(4-methoxyphenyl)hydrazono]-3-oxopropanoate. 846 mg (68% purity, 61% yield) of the title compound were obtained.

LC-MS (Method 8): Rₜ=1.07 min; MS (ESIpos): m/z=273 [M+H]⁺

### Intermediate 185

### Ethyl (2E,3E)-2-[2-(4-chloro-3-fluorophenyl)hydrazinylidene]-3-(hydroxyimino)propanoate

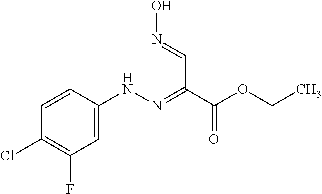 [SMILES: CCOC(=O)C(/C=N/O)=N/Nc1ccc(Cl)c(F)c1]

The reaction was carried out analogously to the preparation of ethyl (2E,3E)-3-(hydroxyimino)-2-[(4-methoxyphenyl)hydrazono]propanoate. 801 mg (51% purity, 68% yield) of the title compound were obtained.

LC-MS (Method 8): Rₜ=1.03 min; MS (ESIpos): m/z=288 [M+H]⁺

### Intermediate 186

### Ethyl 2-(4-chloro-3-fluorophenyl)-2H-1,2,3-triazole-4-carboxylate

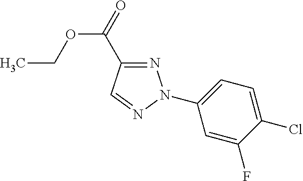 [SMILES: CCOC(=O)c1cnn(-c2ccc(Cl)c(F)c2)n1]

The reaction was carried out analogously to the preparation of ethyl 2-(4-methoxyphenyl)-2H-1,2,3-triazole-4-carboxylate. 278 mg (92% purity, 68% yield) of the title compound were obtained.

LC-MS (Method 7): Rₜ=2.18 min; MS (ESIpos): m/z=270 [M+H]⁺

¹H-NMR (400 MHz, DMSO-d6) δ [ppm]: −0.008 (1.27), 0.008 (1.09), 1.150 (0.53), 1.290 (0.85), 1.308 (1.64), 1.311 (0.87), 1.331 (7.68), 1.349 (16.00), 1.366 (7.66), 2.524 (0.72), 4.370 (2.51), 4.387 (7.55), 4.405 (7.36), 4.423 (2.31), 7.478 (0.43), 7.499 (0.44), 7.833 (1.53), 7.855 (2.83), 7.875 (2.38), 7.943 (1.82), 7.947 (1.93), 7.965 (1.22), 7.969 (1.28), 8.075 (1.63), 8.081 (1.49), 8.100 (1.69), 8.106 (1.54), 8.663 (4.83), 11.807 (0.43).

### Intermediate 187

### 2-(4-chloro-3-fluorophenyl)-2H-1,2,3-triazole-4-carboxylic acid

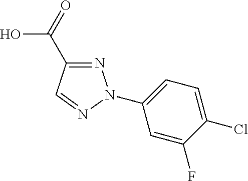 [SMILES: O=C(O)c1cnn(-c2ccc(Cl)c(F)c2)n1]

The reaction was carried out analogously to the preparation of 2-(4-methoxyphenyl)-2H-1,2,3-triazole-4-carboxylic acid. 151 mg (100% purity, 67% yield) of the title compound were obtained.

LC-MS (Method 7): Rₜ=1.53 min; MS (ESIneg): m/z=240 [M−H]⁻

### Intermediate 188

### Ethyl (2E)-2-[2-(2,4-difluorophenyl)hydrazinylidene]-3-oxopropanoate

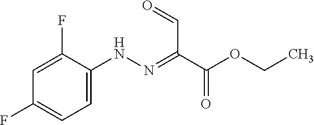 [SMILES: CCOC(=O)/C(C=O)=N/Nc1ccc(F)cc1F]

2,4-Difluoroaniline (500 mg, 3.87 mmol) was dissolved in 5 ml of water and 1 ml of concentrated hydrochloric acid. The mixture was cooled to 0° C. and a solution of sodium nitrite (267 mg, 3.87 mmol) in 2.5 ml of water was added dropwise. After stirring for 5 minutes this solution was added dropwise to a solution of ethyl-(2E)-3-(dimethylamino)acrylate (610 μl, 4.26 mmol) and potassium acetate (570 mg, 5.81 mmol) in 7.5 ml of ethanol at 0° C. The mixture was stirred at room temperature for 10 minutes before being diluted with ethyl acetate and washed twice with water and once with brine. The organic layer was dried over sodium sulfate, filtered and evaporated. 950 mg (83% purity, 80% yield) of the title compound were obtained.

LC-MS (Method 7): Rₜ=1.93 min; MS (ESIpos): m/z=257 [M+H]⁺

### Intermediate 189

### Ethyl (2E,3E)-2-[2-(2,4-difluorophenyl)hydrazinylidene]-3-(hydroxyimino)propanoate

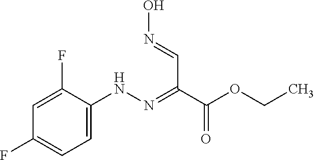 [SMILES: CCOC(=O)C(/C=N/O)=N/Nc1ccc(F)cc1F]

Ethyl (2E)-2-[2-(2,4-difluorophenyl)hydrazinylidene]-3-oxopropanoate (950 mg, 83% purity, 3.08 mmol), hydroxylamine hydrochloride (256 mg, 3.69 mmol) and potassium acetate (755 mg, 7.69 mmol) were dissolved in 10 ml of ethanol and the mixture was stirred for 30 minutes at 80° C. The reaction mixture was diluted with ethyl acetate and washed twice with water and once with brine. The organic layer was dried over sodium sulfate, filtered and evaporated. The residue was dried in vacuum. 950 mg (80% purity, 92% yield) of the title compound were obtained.

LC-MS (Method 7): Rₜ=1.82 min; MS (ESIpos): m/z=272 [M+H]⁺

### Intermediate 190

### Ethyl 2-(2,4-difluorophenyl)-2H-1,2,3-triazole-4-carboxylate

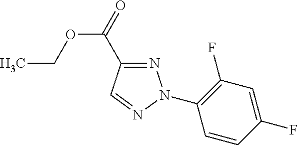 [SMILES: CCOC(=O)c1cnn(-c2ccc(F)cc2F)n1]

Ethyl (2E,3E)-2-[2-(2,4-difluorophenyl)hydrazinylidene]-3-(hydroxyimino)propanoate (950 mg, 2.80 mmol) were stirred for 30 minutes in 11.5 ml of acetic acid anhydride at 140° C. The reaction was then treated with water and extracted with dichlormethane. The organic layer was dried over sodium sulfate, filtered and evaporated. The residue was put on Isolute and purified by chromatography on silica gel (50 g Ultra Snap Cartridge Biotage®; Biotage-Isolera-One®; Cy/EE-gradient: 5% EE-40% EE; flow: 100 ml/min). Samples containing the desired product were united, the solvents were evaporated and the residue was dried in vacuum. 605 mg (85% yield) of the title compound were obtained.

¹H-NMR (400 MHz, DMSO-d6) δ [ppm]: 1.297 (0.53), 1.300 (0.44), 1.315 (1.20), 1.318 (1.17), 1.324 (7.55), 1.333 (0.99), 1.342 (16.00), 1.360 (7.80), 2.025 (0.44), 2.223 (0.83), 2.226 (0.56), 4.292 (0.45), 4.310 (0.45), 4.363 (2.45), 4.381 (7.52), 4.399 (7.41), 4.417 (2.35), 7.340 (0.57), 7.343 (0.68), 7.346 (0.75), 7.350 (0.68), 7.363 (1.28), 7.366 (1.45), 7.369 (1.36), 7.382 (0.67), 7.386 (0.76), 7.389 (0.82), 7.392 (0.72), 7.661 (0.77), 7.668 (0.77), 7.684 (0.88), 7.691 (1.47), 7.696 (0.92), 7.712 (0.81), 7.719 (0.82), 7.946 (1.01), 7.961 (1.10), 7.968 (1.88), 7.983 (1.89), 7.991 (1.03), 8.005 (0.95), 8.647 (4.18), 9.860 (0.43).

### Intermediate 191

### 2-(2,4-difluorophenyl)-2H-1,2,3-triazole-4-carboxylic acid

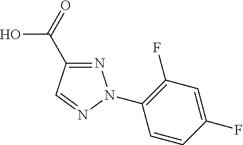 [SMILES: O=C(O)c1cnn(-c2ccc(F)cc2F)n1]

Ethyl 2-(2,4-difluorophenyl)-2H-1,2,3-triazole-4-carboxylate (605 mg, 2.39 mmol) were dissolved in 20 ml of THF, treated with lithium hydroxide solution (2.39 ml, 1.0 M, 2.39 mmol) and the mixture was stirred at room temperature for 2 h. The reaction was then acidified with 2 N hydrochloric acid and put on Isolute before being purified by chromatography on silica gel (25 g Ultra Snap Cartridge Biotage®; Biotage-Isolera-One®; DCM/MeOH-gradient: 2% MeOH-20% MeOH; flow: 75 ml/min). Samples containing the desired product were united and the solvents were evaporated. The residue was taken up with DCM, the precipitate was filtered off, washed with DCM and dried in vacuum. 211 mg (100% purity, 39% yield) of the title compound were obtained.

LC-MS (Method 7): Rₜ=1.15 min; MS (ESIneg): m/z=224 [M−H]⁻

### Intermediate 192

### Ethyl (2E)-2-[2-(3,4-dichlorophenyl)hydrazinylidene]-3-oxopropanoate

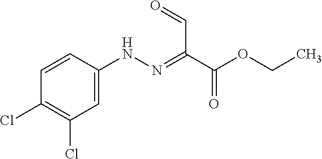 [SMILES: CCOC(=O)/C(C=O)=N/Nc1ccc(Cl)c(Cl)c1]

The reaction was carried out analogously to the preparation of ethyl (2E)-2-[(4-methoxyphenyl)hydrazono]-3-oxopropanoate. 570 mg (71% purity, 45% yield) of the title compound were obtained.

LC-MS (Method 8): Rₜ=1.13 min; MS (ESIpos): m/z=289 [M+H]⁺

### Intermediate 193

### Ethyl (2E,3E)-2-[2-(3,4-dichlorophenyl)hydrazinylidene]-3-(hydroxyimino)propanoate

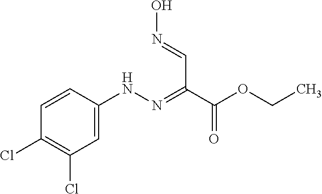 [SMILES: CCOC(=O)C(/C=N/O)=N/Nc1ccc(Cl)c(Cl)c1]

The reaction was carried out analogously to the preparation of ethyl (2E,3E)-3-(hydroxyimino)-2-[(4-methoxyphenyl)hydrazono]propanoate. 429 mg (100% purity, 101% yield) of the title compound were obtained.

LC-MS (Method 7): Rₜ=2.07 min; MS (ESIpos): m/z=304 [M+H]⁺

### Intermediate 194

### Ethyl 2-(3,4-dichlorophenyl)-2H-1,2,3-triazole-4-carboxylate

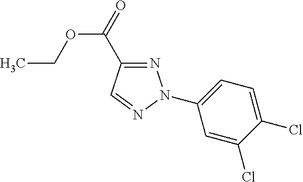 [SMILES: CCOC(=O)c1cnn(-c2ccc(Cl)c(Cl)c2)n1]

The reaction was carried out analogously to the preparation of ethyl 2-(4-methoxyphenyl)-2H-1,2,3-triazole-4-carboxylate. 140 mg (92% purity, 32% yield) of the title compound were obtained.

LC-MS (Method 7): Rₜ=2.34 min; MS (ESIpos): m/z=286 [M+H]⁺

### Intermediate 195

### 2-(3,4-dichlorophenyl)-2H-1,2,3-triazole-4-carboxylic acid

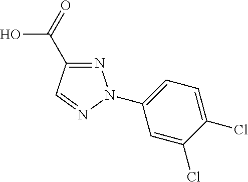 [SMILES: O=C(O)c1cnn(-c2ccc(Cl)c(Cl)c2)n1]

The reaction was carried out analogously to the preparation of 2-(4-methoxyphenyl)-2H-1,2,3-triazole-4-carboxylic acid. 102 mg (100% purity, 88% yield) of the title compound were obtained.

LC-MS (Method 7): Rₜ=1.69 min; MS (ESIneg): m/z=255 [M−H]⁻

### Intermediate 196

### Ethyl (2E)-2-[2-(4-bromophenyl)hydrazinylidene]-3-oxopropanoate

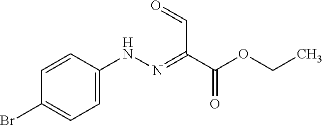 [SMILES: CCOC(=O)/C(C=O)=N/Nc1ccc(Br)cc1]

The reaction was carried out analogously to the preparation of ethyl (2E)-2-[(4-methoxyphenyl)hydrazono]-3-oxopropanoate. 844 mg (85% purity, 83% yield) of the title compound were obtained.

LC-MS (Method 9): Rₜ=1.78 min; MS (ESIpos): m/z=299 [M+H]⁺

### Intermediate 197

### Ethyl (2E,3E)-2-[2-(4-bromophenyl)hydrazinylidene]-3-(hydroxyimino)propanoate

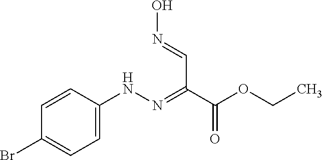 [SMILES: CCOC(=O)C(/C=N/O)=N/Nc1ccc(Br)cc1]

The reaction was carried out analogously to the preparation of ethyl (2E,3E)-3-(hydroxyimino)-2-[(4-methoxyphenyl)hydrazono]propanoate. 873 mg (41% purity, 48% yield) of the title compound were obtained.

LC-MS (Method 7): Rₜ=1.95 min; MS (ESIpos): m/z=314 [M+H]⁺

### Intermediate 198

### Ethyl 2-(4-bromophenyl)-2H-1,2,3-triazole-4-carboxylate

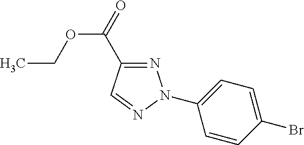 [SMILES: CCOC(=O)c1cnn(-c2ccc(Br)cc2)n1]

The reaction was carried out analogously to the preparation of ethyl 2-(4-methoxyphenyl)-2H-1,2,3-triazole-4-carboxylate. 270 mg (92% purity, 72% yield) of the title compound were obtained.

LC-MS (Method 7): Rₜ=2.18 min; MS (ESIpos): m/z=296 [M+H]⁺

### Intermediate 199

### 2-(4-bromophenyl)-2H-1,2,3-triazole-4-carboxylic acid

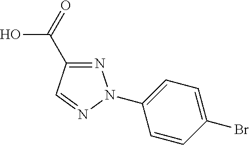 [SMILES: O=C(O)c1cnn(-c2ccc(Br)cc2)n1]

The reaction was carried out analogously to the preparation of 2-(4-methoxyphenyl)-2H-1,2,3-triazole-4-carboxylic acid. 162 mg (100% purity, 73% yield) of the title compound were obtained.

LC-MS (Method 7): Rₜ=1.53 min; MS (ESIneg): m/z=265 [M−H]⁻

### Intermediate 200

### tert-butyl {R4R)-4-cyclopropyl-2,5-dioxoimidazolidin-4-yl)methyl}carbamate

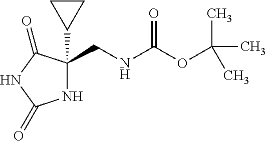 [SMILES: CC(C)(C)OC(=O)NC[C@@]1(C2CC2)NC(=O)NC1=O]

rac-tert-Butyl [(4-cyclopropyl-2,5-dioxoimidazolidin-4-yl)methyl]carbamate (26 g) was dissolved in 2600 ml of Methanol at 50° C., filtered and separated into it's enantiomers by chiral chromatography (Machine: SFC Prep Sepiatec 360; Column: Chiralpak AD 20μ, 450×50 mm; Eluent: CO₂/Methanol 70:30; Flow: 350 ml/min; UV Detection: 210 nm; Backpressure: 130 bar; Oven temperature: 35° C.). The enantiomer eluting at 1.52 minutes was collected. Enantiomeric purity was determined on an analytical scale (Column: Chiralpak AD-H 5μ, 250×4.6 mm; Eluent: CO₂/Methanol 80:20; Flow: 3 ml/min; UV Detection: 210 nm) as 99.9% ee. 8.6 g (33% yield).

LC-MS (Method 8): Rₜ=0.60 min; MS (ESIpos): m/z=270 [M+H]⁺

The absolute stereochemistry was deduced by deprotection (see next intermediate) and synthesis of example 3 from WO2014066151.

### Intermediate 201

### (5R)-5-(aminomethyl)-5-cyclopropylimidazolidine-2,4-dione hydrochloride

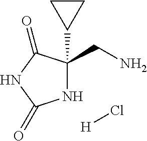

tert-butyl {[(4R)-4-cyclopropyl-2,5-dioxoimidazolidin-4-yl]methyl}carbamate (6 g, 22.28 mmol) was dissolved in 70 ml of dichloromethane and treated with 4N HCl in dioxane (27.85 ml, 111.4 mmol). The mixture was stirred at room temperature over night. The precipitate was filtered off, washed with dichloromethane and dried in vacuo. 4.29 g (94% yield, 100% purity) of the title compound were obtained.

LC-MS (Method 9): Rₜ=0.27 min; MS (ESIpos): m/z=170 [M−HCl+H]⁺

Specific optical rotation: −75.92° (Solvent: methanol; Pathlength: 100 mm; Concentration: 0.2665 g/100 cm³; Temperature: 20° C.; Wavelength: 589 nm)

### Intermediate 202

### Ethyl 5-(5-methyl-1,3-thiazol-4-yl)-1,3-oxazole-4-carboxylate

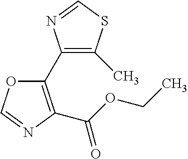 [SMILES: CCOC(=O)c1ncoc1-c1ncsc1C]

5-methyl-1,3-thiazole-4-carboxylic acid (940 mg, 6.57 mmol) dissolved in 10 ml of THF was treated with 1,1,-carbonyl-diimidazole (1.28 g, 7.88 mmol) and stirred at room temperature. After 2 h ethyl isocyanoacetate (790 μl, 7.2 mmol), dissolved in 10 ml of THF, and a solution of lithium bis(trimethylsilyl)amide in THF (6.6 ml, 1.0 M, 6.6 mmol) were added at 0° C. The mixture was allowed to warm to room temperature and stirred over night. The solvent was removed on a rotary evaporator. The residue was taken up with ethyl acetate and washed with water and saturated aqueous sodium chloride solution. The organic layer was dried over sodium sulfate, filtered, Isolute® was added and the solvent was evaporated on a rotary evaporator. The crude product was purified by column chromatography (Machine: Biotage® Isolera One; column: Biotage® SNAP Ultra 50 g; gradient: Cy/EE-gradient, 16% EE-100% EE; flow: 100 ml/min). Product containing samples were united and the solvents were removed on a rotary evaporator. 1.13 g (100% purity, 72% yield) of the title compound were obtained.

LC-MS (Method 7): Rₜ=1.15 min; MS (ESIpos): m/z=239 [M+H]⁺

### Intermediate 203

### 2-amino-1-(5-methyl-1,3-thiazol-4-yl)ethan-1-one-hydrogen chloride (1/1)

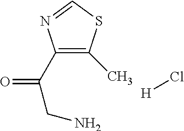

Ethyl 5-(5-methyl-1,3-thiazol-4-yl)-1,3-oxazole-4-carboxylate (1.13 g, 4.74 mmol) was taken up in 25 ml of 6 N hydrochloric acid and stirred at 100° C. After 2 h the solvent was removed on a rotary evaporator and the residue was treated with DCM/MeOH 20:1. The precipitate was filtered off, washed with DCM/MeOH 20:1 and dried in vacuo. 730 mg (98% purity, 79% yield) of the title compound were obtained.

LC-MS (Method 9): Rₜ=0.80 min; MS (ESIpos): m/z=157 [M−HCl+H]⁺

### Intermediate 204

### tert-butyl [2-(5-methyl-1,3-thiazol-4-yl)-2-oxoethyl]carbamate

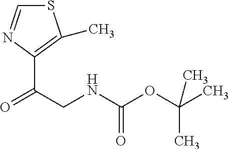 [SMILES: Cc1scnc1C(=O)CNC(=O)OC(C)(C)C]

2-amino-1-(5-methyl-1,3-thiazol-4-yl)ethan-1-one-hydrogen chloride (730 mg, 3.79 mmol) dissolved in 15 ml of dichloromethane was treated with di-tert-butyl dicarbonate (960 μl, 4.2 mmol) and triethylamine (1.6 ml, 11 mmol). The mixture was stirred at room temperature. After 2 h the solvent was removed on a rotary evaporator. The residue was taken up with ethyl acetate and washed with water and saturated aqueous sodium chloride solution. The organic layer was dried over sodium sulfate, filtered and evaporated on a rotary evaporator. 980 mg (100% purity, 101% yield) of the title compound were obtained.

LC-MS (Method 7): Rₜ=1.57 min; MS (ESIpos): m/z=257 [M+H]⁺

### Intermediate 205

### rac-tert-butyl {[4-(5-methyl-1,3-thiazol-4-yl)-2,5-dioxoimidazolidin-4-yl]methyl}carbamate

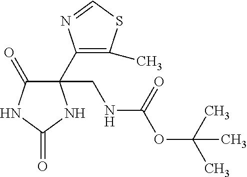 [SMILES: Cc1scnc1C1(CNC(=O)OC(C)(C)C)NC(=O)NC1=O]

In a microwave vial tert-butyl [2-(5-methyl-1,3-thiazol-4-yl)-2-oxoethyl]carbamate (980 mg, 3.82 mmol) was dissolved in 7 ml of methanol. Potassium cyanide (996 mg, 15.3 mmol) and ammonium carbonate (1.47 g, 15.3 mmol) were added. The vial was sealed and the mixture was stirred at 40° C. for 48 h. Further potassium cyanide (996 mg, 15.3 mmol) and ammonium carbonate (1.47 g, 15.3 mmol) were added and the mixture was stirred for 72 h. Isolute® was added and the solvent was evaporated on a rotary evaporator. The crude product was purified by column chromatography (Machine: Biotage® Isolera One; column: Biotage® SNAP Ultra 50 g; gradient: DCM/MeOH-gradient, 2% MeOH-20% MeOH; flow: 100 ml/min). Product containing samples were united and the solvents were removed on a rotary evaporator. 766 mg (100% purity, 61% yield) of the title compound were obtained.

LC-MS (Method 7): Rₜ=1.09 min; MS (ESIneg): m/z=325 [M−H]⁻

### Intermediate 206

### rac-5-(aminomethyl)-5-(5-methyl-1,3-thiazol-4-yl)imidazolidine-2,4-dione hydrochloride

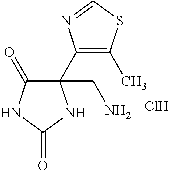

rac-tert-butyl {[4-(5-methyl-1,3-thiazol-4-yl)-2,5-dioxoimidazolidin-4-yl]methyl}carbamate (766 mg, 2.35 mmol) was dissolved in 9 ml of dichloromehane. 4 M hydrochloride acid in 1,4-dioxane (2.9 ml, 4.0 M, 12 mmol) was added and the mixture was stirred for 30 min. The precipitate was filtered off, washed with dichloromethane and dried in vacuo. 640 mg (86% purity, 89% yield) of the title compound were obtained.

LC-MS (Method 9): Rₜ=0.25 min; MS (ESIpos): m/z=227 [M−HCl+H]⁺

### Intermediate 207

### 3-fluoro-2-hydrazinylpyridine

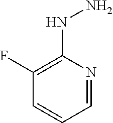

2,3-difluoropyridine (1.00 g, 8.69 mmol) were dissolved in hydrazine hydrate (4.2 ml, 87 mmol). The mixture was stirred at 120° C. for 1 h. The mixture was extracted with dichloromethane, the organic layer was dried over sodium sulfate, filtered and evaporated on a rotary evaporator. 1.03 g (93% yield) of the title compound were obtained.

### Intermediate 208

### N-{(1E,2Z)-2-[2-(3-fluoropyridin-2-yl)hydrazinylidene]propylidene}hydroxylamine

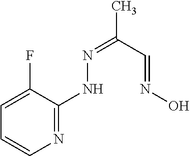

3-fluoro-2-hydrazinylpyridine (1.03 g, 8.10 mmol) was dissolved in 35 ml of ethanol. (1E)-1-(hydroxyimino)propan-2-one (847 mg, 9.72 mmol) was added and the mixture was stirred at 80° C. After 2 h the solvent was removed on a rotary evaporator. The residue was taken up with pentane. The precipitate was filtered off, washed with pentane and dried in vacuo. 1.64 g (103% yield) of the title compound were obtained.

### Intermediate 209

### 3-fluoro-2-(4-methyl-2H-1,2,3-triazol-2-yl)pyridine

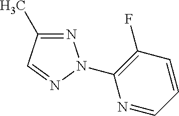

N-{(1E,2Z)-2-[2-(3-fluoropyridin-2-yl)hydrazinylidene]propylidene}hydroxylamine (1.64 g, 8.36 mmol) was dissolved in 30 ml of acetic anhydride and stirred at 130° C. After 3 h Isolute® was added and the solvent was evaporated on a rotary evaporator. The crude product was purified by column chromatography (Machine: Biotage® Isolera One; column: Biotage® SNAP Ultra 50 g; gradient: Cy/EE-gradient, 12% EE-100% EE; flow: 100 ml/min). Product containing samples were united and the solvents were removed on a rotary evaporator. 844 mg (79% purity, 45% yield) of the title compound were obtained.

LC-MS (Method 7): Rₜ=1.03 min; MS (ESIpos): m/z=179 [M+H]⁺

¹H-NMR (400 MHz, DMSO-d6) δ [ppm]: 2.387 (16.00), 2.509 (1.26), 3.327 (2.56), 7.653 (0.56), 7.664 (0.93), 7.674 (1.21), 7.685 (1.05), 7.694 (0.65), 7.994 (2.43), 8.071 (0.82), 8.094 (1.10), 8.097 (0.95), 8.118 (0.78), 8.450 (1.33), 8.461 (1.32).

### Intermediate 210

### 2-(3-fluoropyridin-2-yl)-2H-1,2,3-triazole-4-carboxylic acid

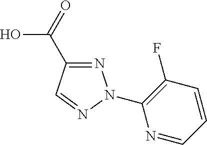 [SMILES: O=C(O)c1cnn(-c2ncccc2F)n1]

3-fluoro-2-(4-methyl-2H-1,2,3-triazol-2-yl)pyridine (740 mg, 4.15 mmol) was suspended in 45 ml of water. Potassium permanganate (1.31 g, 8.31 mmol) were added and the mixture was stirred at 100° C. After 24 h more potassium permanganate (500 mg, 3.16 mmol) were added and the mixture was stirred at 100° C. for 24 h. Once again more potassium permanganate (500 mg, 3.16 mmol) was added and the mixture was stirred for another 72 h at 100° C. The mixture was filtered over Celite® and the filtrate was acidified with 2 N hydrochloric acid. The solvent was removed on a rotary evaporator. Dichloromethane/methanol 10:1 were added to the crude product and the precipitate was filtered off. The filtrate was purified by preparative HPLC (Column: Chromatorex C18 10 μm, 250×30 mm; eluent A=water, B=acetonitrile; gradient: 0.0 min 5% B; 3 min 5% B; 20 min 50% B; 23 min 100% B; 26 min 5% B; flow: 50 ml/min; 0.1% formic acid). 149 mg (100% purity, 17% yield) of the title compound were obtained.

LC-MS (Method 7): Rₜ=0.63 min; MS (ESIpos): m/z=209 [M+H]⁺

### Intermediate 211

### rac-Ethyl 5-sec-butyl-1,3-oxazole-4-carboxylate

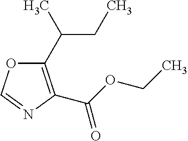

A solution of rac-2-methylbutanoic acid (1.1 ml, 9.8 mmol) and 1,1′-carbonyldiimidazole (1.91 g, 11.7 mmol) in 10 ml of THF was stirred for 3 h at room temperature. After that time, a solution of ethyl isocyanoacetate (1.2 ml, 11 mmol) in 10 ml of THF and a solution of lithium bis(trimethylsilyl)amide in THF (9.8 ml, 1.0 M, 9.8 mmol) were added dropwise at 0° C. When the addition was complete the resulting mixture was stirred at room temperature for 3 h. The reaction was concentrated in vacuo and extracted with ethyl acetate. The combined organic layers were dried over sodium sulfate, filtered and concentrated. The crude product was purified by column chromatography (Machine: Biotage® Isolera One; column: Biotage® SNAP Ultra 50 g; gradient: Cy/EE-gradient, 8% EE-66% EE; flow: 100 ml/min). Product containing samples were united and the solvents were removed on a rotary evaporator. 446 mg (97% purity, 22% yield) of the title compound were obtained.

LC-MS (Method 7): Rₜ=1.70 min; MS (ESIpos): m/z=198 [M+H]⁺

### Intermediate 212

### rac-1-amino-3-methylpentan-2-one hydrochloride

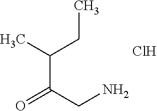

rac-ethyl 5-sec-butyl-1,3-oxazole-4-carboxylate (446 mg, 2.26 mmol) were taken up in 7.5 ml of 6 N hydrochloric acid and stirred at 100° C. over night. The solvent was removed on a rotary evaporator and the residue was dried in vacuo. 372 mg (108% yield) of the title compound were obtained.

### Intermediate 213

### rac-tert-butyl (3-methyl-2-oxopentyl)carbamate

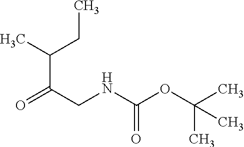

rac-1-amino-3-methylpentan-2-one hydrochloride (372 mg, 2.45 mmol) dissolved in 15 ml of dichloromethane were treated with di-tert-butyl dicarbonate (620 μl, 2.7 mmol) and triethylamine (1.0 ml, 7.4 mmol). The mixture was stirred at room temperature over night. The solvent was removed on a rotary evaporator. The residue was taken up with ethyl acetate and washed with water and saturated aqueous sodium chloride solution. The organic layer was dried over sodium sulfate, filtered and evaporated on a rotary evaporator. 480 mg (100% purity, 91% yield) of the title compound were obtained.

¹H-NMR (400 MHz, DMSO-d6) δ [ppm]: 0.786 (2.10), 0.804 (4.90), 0.823 (2.39), 0.958 (3.46), 0.975 (3.56), 1.290 (0.48), 1.308 (0.75), 1.324 (1.46), 1.343 (0.69), 1.366 (2.11), 1.377 (16.00), 1.468 (1.14), 1.532 (0.41), 1.550 (0.50), 1.567 (0.50), 1.584 (0.41), 2.519 (0.94), 3.778 (0.94), 3.789 (1.12), 3.793 (1.06), 3.804 (0.89), 6.977 (0.42).

### Intermediate 214

### diamix-tert-butyl [(4-sec-butyl-2,5-dioxoimidazolidin-4-yl)methyl]carbamate

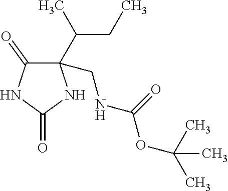 [SMILES: CCC(C)C1(CNC(=O)OC(C)(C)C)NC(=O)NC1=O]

In a microwave vial rac-tert-butyl (3-methyl-2-oxopentyl)carbamate (480 mg, 2.23 mmol) was dissolved in 4 ml of methanol. Potassium cyanide (581 mg, 8.92 mmol) and ammonium carbonate (857 mg, 8.92 mmol) were added. The vial was sealed and the mixture was stirred at 40° C. over night. The mixture was filtered an the filtrate was purified by preparative HPLC (Column: Chromatorex C18 10 μm 250×30 mm; eluent A=water, B=acetonitrile; gradient: 0.0 min 30% B; 4.5 min 50% B; 11.5 min 70% B; 12 min 100% B; 14.75 min 30% B; flow: 50 ml/min; 0.1% formic acid). Product containing samples were united and the solvents were removed on a rotary evaporator. 237 mg (100% purity, 37% yield) of the title compound were obtained.

LC-MS (Method 7): Rₜ=1.27 min; MS (ESIneg): m/z=284 [M−H]⁻

LC-MS (Method 8): Rₜ=0.74 min; MS (ESIneg): m/z=284 [M−H]⁻

### Intermediate 215

### diamix-5-(aminomethyl)-5-sec-butylimidazolidine-2,4-dione hydrochloride

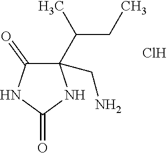

diamix-tert-butyl [(4-sec-butyl-2,5-dioxoimidazolidin-4-yl)methyl]carbamate (235 mg, 824 μmol) was dissolved in 3 ml of dichloromethane. 4 M hydrochloric acid in 1,4-dioxane (1.0 ml, 4.0 M, 4.1 mmol) was added and the mixture was stirred for 4 h. The solvent was removed on a rotary evaporator. The residue was taken up in dichloromethane, the precipitate was filtered off, washed with dichloromethane and dried in vacuo. 155 mg (100% purity, 85% yield) of the title compound were obtained.

LC-MS (Method 9): Rₜ=0.36 min; MS (ESIpos): m/z=186 [M−HCl+H]⁺

### Intermediate 216

### Ethyl 2-(1,3-benzothiazol-2-yl)-2H-1,2,3-triazole-4-carboxylate

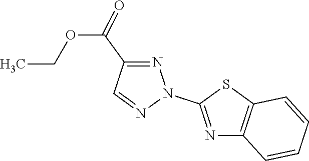 [SMILES: CCOC(=O)c1cnn(-c2nc3ccccc3s2)n1]

To a solution of ethyl 2H-1,2,3-triazole-4-carboxylate (165 mg, 1.17 mmol) and 2-bromo-1,3-benzothiazole (250 mg, 1.17 mmol) in 5 ml of DMF sodium hydrogen carbonate (196 mg, 2.34 mmol) was added and the mixture was stirred for 72 h at 60° C. The precipitate was filtered off and the filtrate was purified by preparative HPLC (Column: Chromatorex C18 10 μm 250×30 mm; eluent A=water, B=acetonitrile; gradient: 0.0 min 30% B; 4.5 min 50% B; 11.5 min 70% B; 12 min 100% B; 14.75 min 30% B; flow: 50 ml/min; 0.1% formic acid). Product containing samples were united and the solvents were removed on a rotary evaporator. 46.8 mg (100% purity, 15% yield) of the title compound were obtained.

LC-MS (Method 7): Rₜ=1.91 min; MS (ESIpos): m/z=275 [M+H]⁺

¹H-NMR (600 MHz, DMSO-d6) δ [ppm]: 1.361 (7.74), 1.372 (16.00), 1.384 (7.83), 4.407 (2.50), 4.419 (7.64), 4.431 (7.48), 4.443 (2.38), 7.546 (1.58), 7.560 (3.38), 7.572 (2.30), 7.617 (2.09), 7.618 (2.00), 7.630 (3.35), 7.642 (1.63), 8.079 (3.55), 8.093 (3.41), 8.210 (3.37), 8.224 (3.28), 8.798 (9.95).

### Intermediate 217

### 2-(1,3-benzothiazol-2-yl)-2H-1,2,3-triazole-4-carboxylic acid

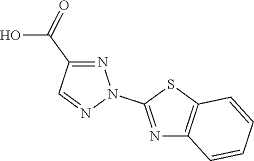 [SMILES: O=C(O)c1cnn(-c2nc3ccccc3s2)n1]

Ethyl 2-(1,3-benzothiazol-2-yl)-2H-1,2,3-triazole-4-carboxylate (46.0 mg, 168 μmol) was dissolved in 2 ml of THF, treated with lithium hydroxide solution (200 μl, 1.0 M, 200 μmol) and the mixture was stirred for 2 h at room temperature. The solvent was removed on a rotary evaporator. Water was added and the mixture was acidified with 2 N hydrochloric acid. The precipitate was filtered off, washed with water an dried in vacuo. 36.0 mg (100% purity, 87% yield) of the title compound were obtained.

LC-MS (Method 8): Rₜ=0.75 min; MS (ESIpos): m/z=247 [M+H]⁺

### Intermediate 218

### Ethyl (2E)-3-oxo-2-{2-[4-(trifluoromethoxy)phenyl]hydrazinylidene}propanoate

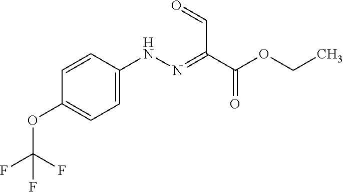 [SMILES: CCOC(=O)/C(C=O)=N/Nc1ccc(OC(F)(F)F)cc1]

4-(trifluoromethoxy)aniline (250 mg, 1.41 mmol) was dissolved in 2.5 ml of water and 380 μl of concentrated hydrochloric acid. The mixture was cooled to 0° C. and a solution of sodium nitrite (97.4 mg, 1.41 mmol) in 1.25 ml of water was added dropwise. After stirring for 5 minutes this solution was added dropwise to a solution of ethyl-(2E)-3-(dimethylamino)acrylate (220 μl, 1.6 mmol) and potassium acetate (208 mg, 2.12 mmol) in 3.6 ml of ethanol at 0° C. The mixture was stirred at room temperature for 30 min before being diluted with ethyl acetate and washed twice with water and once with brine. The organic layer was dried over sodium sulfate, filtered and evaporated. 394 mg (92% yield) of the title compound were obtained.

### Intermediate 219

### Ethyl (2E,3E)-3-(hydroxyimino)-2-{2-[4-(trifluoromethoxy)phenyl]hydrazinylidene}propanoate

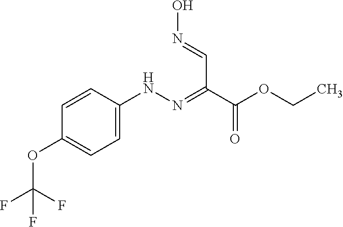 [SMILES: CCOC(=O)C(/C=N/O)=N/Nc1ccc(OC(F)(F)F)cc1]

Ethyl (2E)-3-oxo-2-{2-[4-(trifluoromethoxy)phenyl]hydrazinylidene}propanoate (393 mg, 1.29 mmol), hydroxylamine hydrochloride (108 mg, 1.55 mmol) and potassium acetate (317 mg, 3.23 mmol) were dissolved in 7 ml of ethanol and the mixture was stirred for 3 h at room temperature. The reaction mixture was diluted with ethyl acetate and washed twice with water and once with brine. The organic layer was dried over sodium sulfate, filtered and evaporated. The residue was dried in vacuum. 350 mg (39% purity, 33% yield) of the title compound were obtained.

LC-MS (Method 7): Rₜ=2.02 min; MS (ESIpos): m/z=320 [M+H]⁺

### Intermediate 220

### Ethyl 2-[4-(trifluoromethoxy)phenyl]-2H-1,2,3-triazole-4-carboxylate

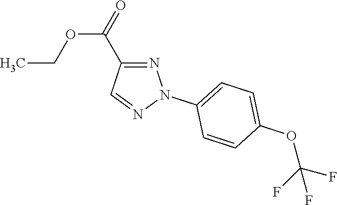 [SMILES: CCOC(=O)c1cnn(-c2ccc(OC(F)(F)F)cc2)n1]

Ethyl (2E,3E)-3-(hydroxyimino)-2-{2-[4-(trifluoromethoxy)phenyl]hydrazinylidene}propanoate (350 mg, 1.10 mmol) was stirred for 1.5 h in 5 ml of acetic acid anhydride at 60° C. The reaction was then diluted in ethyl acetate and washed twice with water and once with brine. The organic layer was dried over sodium sulfate, filtered and evaporated. The residue was put on Isolute® and purified by chromatography on silica gel (25 g Ultra Snap Cartridge Biotage®; Biotage-Isolera-One®; Cy/EE-gradient: 12% EE-100% EE; flow: 75 ml/min). Samples containing the desired product were united, the solvents were evaporated and the residue was dried in vacuum. 95.6 mg (58% purity, 17% yield) of the title compound were obtained.

LC-MS (Method 7): Rₜ=2.25 min; MS (ESIpos): m/z=302 [M+H]⁺

### Intermediate 221

### 2-[4-(trifluoromethoxy)phenyl]-2H-1,2,3-triazole-4-carboxylic acid

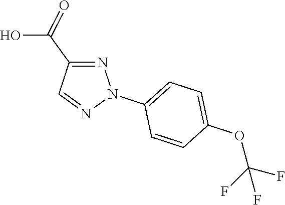 [SMILES: O=C(O)c1cnn(-c2ccc(OC(F)(F)F)cc2)n1]

Ethyl 2-[4-(trifluoromethoxy)phenyl]-2H-1,2,3-triazole-4-carboxylate (95.0 mg, 315 μmol) was dissolved in 2.5 ml of THF, treated with lithium hydroxide solution (320 μl, 1.0 M, 320 μmol) aid the mixture was stirred for 1.5 h at room temperature. The solvent was removed on a rotary evaporator. Water was added an the mixture was acidified with 2 N hydrochloric acid. The precipitate was filtered off, washed with water an dried in vacuo. 14.7 mg (69% purity, 12% yield) of the title compound were obtained

LC-MS (Method 7): Rₜ=1.69 min; MS (ESIneg): m/z=272 [M−H]⁻

### Intermediate 222

### rac-tert-butyl [(4-cyclobutyl-2,5-dioxoimidazolidin-4-yl)methyl]carbamate

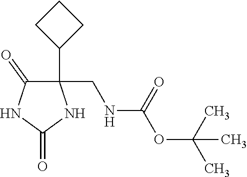 [SMILES: CC(C)(C)OC(=O)NCC1(C2CCC2)NC(=O)NC1=O]

rac-5-(aminomethyl)-5-cyclobutylimidazolidine-2,4-dione-hydrogen chloride (1.00 g, 4.55 mmol) was dissolved in 10 ml of THF and cooled at 0° C. At this temperature triethylamine (1.9 ml, 14 mmol), 4-(dimethylamino)pyridine (83.4 mg, 683 μmol) and di-tert-butyl dicarbonate (1.04 g, 4.78 mmol) were added and the mixture was stirred over night at room temperature. The reaction was then diluted in ethyl acetate and washed twice with water and once with brine. The organic layer was dried over sodium sulfate, filtered and evaporated. The residue was put on Isolute® and purified by chromatography on silica gel (25 g Ultra Snap Cartridge Biotage®; Biotage-Isolera-One®; DCM/MeOH-gradient: 2% MeOH-20% MeOH; flow: 75 ml/min). Samples containing the desired product were united, the solvents were evaporated and the residue was dried in vacuum. 247 mg (100% purity, 19% yield) of the title compound were obtained.

LC-MS (Method 9): Rₜ=0.91 min; MS (ESIneg): m/z=282 [M−H]⁻

### Intermediate 223

### ent-tert-butyl [(4-cyclobutyl-2,5-dioxoimidazolidin-4-yl)methyl]carbamate

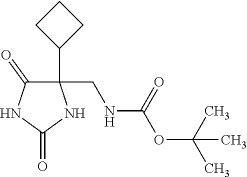 [SMILES: CC(C)(C)OC(=O)NCC1(C2CCC2)NC(=O)NC1=O]

Enantiomeric separation of rac-tert-butyl [(4-cyclobutyl-2,5-dioxoimidazolidin-4-yl)methyl]carbamate (245 mg, 865 μmol) using the following method
Column: Maisch Diacel AD-H 5 μm 250*25 mm
Eluent A: 80% CO₂, eluent B: 20% methanol
Flow: 80 ml/min
UV-detection: 210 nm
Temperature: 40° C.
   afforded 92.9 mg (100% purity, 38% yield) of the desired product.

Chiral-HPLC (Method Info: AD-15 MeOH, 3.0 ml/min; 210 nm, 10 min, 5 μl): Rₜ=1.87 min; 98% ee

LC-MS (Method 7): Rₜ=1.25 min; MS (ESIneg): m/z=282 [M−H]⁻

¹H-NMR (400 MHz, DMSO-d6) δ [ppm]: −0.008 (0.60), 0.008 (0.54), 1.364 (16.00), 1.867 (0.41), 3.085 (0.49), 7.724 (0.80), 10.536 (0.51).

### Intermediate 224

### ent-5-(aminomethyl)-5-cyclobutylimidazolidine-2,4-dione hydrochloride

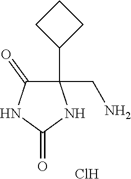 [SMILES: NCC1(C2CCC2)NC(=O)NC1=O ; Cl]

ent-tert-butyl [(4-cyclobutyl-2,5-dioxoimidazolidin-4-yl)methyl]carbamate (92.0 mg, 325 μmol) was dissolved in 1.5 ml of dichloromehane. 4 M hydrochloric acid in 1,4-dioxane (410 μl, 4.0 M, 1.6 mmol) was added and the mixture was stirred over night at room temperature. The solvent was removed on a rotary evaporator and the residue was dried in vacuo. 73.0 mg (100% purity, 102% yield) of the title compound were obtained.

LC-MS (Method 9): Rₜ=0.36 min; MS (ESIpos): m/z=184 [M−HCl+H]⁺

### Intermediate 225

### tert-Butyl (R)-(1-cyclopropyl-1-oxopropan-2-yl)carbamate

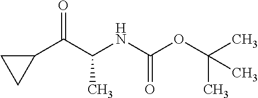

A solution of isopropylmagnesium chloride (3.40 mL, 2.0 M in THF) was added dropwise to a solution of (R)-tert-butyl (1-(methoxy(methyl)amino)-1-oxopropan-2-yl)carbamate (1.50 g, 6.46 mmol) in dry THF (25 mL) at 0° C. under inert atmosphere. After 45 minutes, a solution of cyclopropylmagnesium bromide (15.4 mL, 0.5 M in THF) was added and the reacting mixture was stirred at room temperature for 72 hours. The reaction mixture was quenched by addition of a saturated aqueous ammonium chloride solution followed by extraction with dichloromethane. The combined organic layers were dried over magnesium sulfate, filtered aid concentrated. The residue was purified by column chromatography on silica gel (0 to 30% ethyl acetate in hexanes). 880 mg (90% purity, 64% yield,) of the title compound were obtained.

¹H NMR (400 MHz, Chloroform-d) δ 5.36 (brs, 1H), 4.54 (m, 1H), 2.01 (m, 1H), 1.46 (s, 9H), 1.43 (d, J=7.1 Hz, 3H), 1.17-1.03 (m, 2H), 1.02-0.94 (m, 2H).

### Intermediate 226

### ent-tert-Butyl ((1R)-1-(4-cyclopropyl-2,5-dioxoimidazolidin-4-yl)ethyl)carbamate

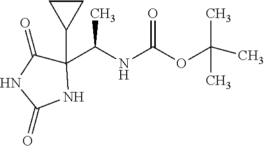 [SMILES: C[C@@H](NC(=O)OC(C)(C)C)C1(C2CC2)NC(=O)NC1=O]

A solution of (R)-tert-butyl (1-cyclopropyl-1-oxopropan-2-yl)carbamate (0.80 g, 3.8 mmol), potassium cyanide (0.73 g, 11 mmol) and ammonium carbonate (1.80 g, 18.8 mmol) in ethanol/water (1:1, 20 mL) was stirred at reflux for 6 days (˜50% conversion, dr=˜2:1). The solvent was removed under reduced pressure and the residue loaded onto silica gel for dry loading and purified via column chromatography on silica gel (35-65% ethyl acetate in hexanes) to afford both diastereomers. 101 mg (95% purity, 13% yield) of diastereomer A and 84 mg (75% purity, 14% yield) of diastereomer B of the title compound were obtained.

Diastereomer A: LC-MS (Method 13): Rₜ=0.96 min; MS (ESIpos): m/z=284 [M+H]⁺

Diastereomer B: LC-MS (Method 13): Rₜ=0.91 min; MS (ESIpos): m/z=284 [M+H]⁺

### Intermediate 227

### ent-5-((R)-1-Aminoethyl)-5-cyclopropylimidazolidine-2,4-dione bis (2,2,2-trifluoroacetate)

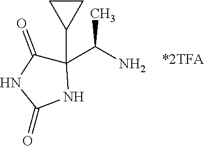

Diastereomer A of ent-tert-Butyl ((1R)-1-(4-cyclopropyl-2,5-dioxoimidazolidin-4-yl)ethyl)carbamate (110 mg) was stirred in a solution of 20% trifluoroacetic acid in dichloromethane (7 mL) at room temperature for 3 hours. The solvent was removed under reduced pressure and the excess trifluoroacetic acid was azeotroped with toluene to afford 5-((R-1-aminoethyl)-5-cyclopropylimidazoline-2,4-dione bis (2,2,2-tridluoroacetate). The material was used in the next step without further purification or determining the yield.

¹H NMR (400 MHz, DMSO-d₆) b 11.07 (brs, 1H), 8.02 (brs, 3H), 3.55 (q, J=6.5, 6.0 Hz, 1H), 1.26 (m, 4H), 0.58 (m, 1H), 0.47 (m, 2H), 0.44 (m, 1H).

### Intermediate 228

### tert-Butyl (S)-(1-cyclopropyl-1-oxopropan-2-yl)carbamate

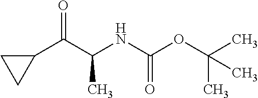

The reaction was carried out analogously to the preparation of tert-butyl (R)-(1-cyclopropyl-1-oxopropan-2-yl)carbamate. 800 mg (90% purity, 60% yield) of the title compound were obtained.

¹H NMR (400 MHz, Chloroform-d) δ 5.36 (brs, 1H), 4.54 (m, 1H), 2.01 (m, 1H), 1.46 (s, 9H), 1.43 (d, J=7.1 Hz, 3H), 1.17-1.03 (m, 2H), 1.02-0.94 (m, 2H).

### Intermediate 229

### ent-tert-Butyl ((1S)-1-(4-cyclopropyl-2,5-dioxoimidazolidin-4-yl)ethyl)carbamate

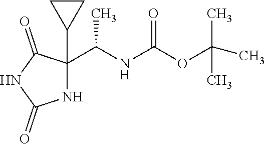 [SMILES: C[C@H](NC(=O)OC(C)(C)C)C1(C2CC2)NC(=O)NC1=O]

The reaction was carried out analogously to the preparation of ent-tert-Butyl ((1R)-1-(4-cyclopropyl-2,5-dioxoimidazolidin-4-yl)ethyl)carbamate. 68 mg (95% purity, 13% yield) of diastereomer A and 147 mg (95% purity, 28% yield) of diastereomer B of the title compound were obtained.

Diastereomer A: LC-MS (Method 13): Rₜ=0.96 min; MS (ESIpos): m/z=282 [M+H]⁺

Diastereomer B: LC-MS (Method 13): Rₜ=0.92 min; MS (ESIpos): m/z=282 [M+H]⁺

### Intermediate 230

### ent-5-((S)-1-Aminoethyl)-5-cyclopropylimidazolidine-2,4-dione bis (2,2,2-trifluoroacetate)

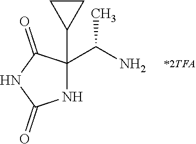

The reaction was carried out analogously to the preparation of ent-5-((R)-1-Aminoethyl)-5-cyclopropylimidazolidine-2,4-dione bis (2,2,2-trifluoroacetate). Using Diastereomer B of ent-tert-Butyl ((1S)-1-(4-cyclopropyl-2,5-dioxoimidazolidin-4-yl)ethyl)carbamate (147 mg), the title cpd was obtained and was used without further purification or determining the yield in the next step.

¹H NMR (400 MHz, DMSO-d6) δ 11.00 (brs, 1H), 8.00 (brs, 3H), 3.62 (m, 1H), 1.25 (d, J=6.2 Hz, 3H), 1.14 (m, 1H), 0.57 (m, 2H), 0.43 (m, 1H), 0.07 (m, 1H).

### Intermediate 231

### Ethyl (2E)-3-oxo-2-[2-(3,4,5-trifluorophenyl)hydrazinylidene]propanoate

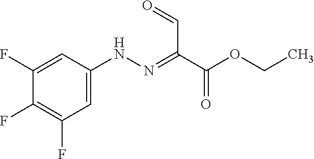 [SMILES: CCOC(=O)/C(C=O)=N/Nc1cc(F)c(F)c(F)c1]

3,4,5-Trifluoroaniline (1.87 g, 12.7 mmol) was dissolved in 20 ml of water and 3.7 ml of concentrated hydrochloric acid. The mixture was cooled to 0° C. and a solution of sodium nitrite (876 mg, 12.7 mmol) in 10 ml of water was added dropwise. After stirring for 5 minutes, this solution was added dropwise to a solution of ethyl-(2E)-3-(dimethylamino)acrylate (2.0 ml, 14 mmol) and potassium acetate (1.87 g, 19.0 mmol) in 30 ml of ethanol at 0° C. The mixture was stirred at room temperature for one hour before being diluted with ethyl acetate and washed twice with water and once with brine. The organic layer was dried over sodium sulfate, filtered and evaporated. 3.20 g (91% purity, 83% yield) of the title compound were obtained.

LC-MS (Method 7): Rₜ=2.00 min; MS (ESIpos): m/z=275 [M+H]⁺

### Intermediate 232

### Ethyl (2E,3E)-3-(hydroxyimino)-2-[2-(3,4,5-trifluorophenyl)hydrazinylidene]propanoate

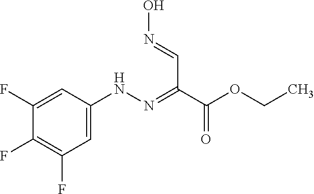 [SMILES: CCOC(=O)C(/C=N/O)=N/Nc1cc(F)c(F)c(F)c1]

Ethyl (2E)-3-oxo-2-[2-(3,4,5-trifluorophenyl)hydrazinylidene]propanoate (3.20 g, 91% purity, 10.6 mmol), hydroxylamine hydrochloride (881 mg, 12.7 mmol) and potassium acetate (2.59 g, 26.4 mmol) were dissolved in 46 ml of ethanol and the mixture was stirred for 30 minutes at 80° C. The reaction mixture was diluted with ethyl acetate and washed twice with water and once with brine. The organic layer was dried over sodium sulfate, filtered and evaporated. The residue was dried in vacuum. 3.17 g (79% purity, 82% yield) of the title compound were obtained

LC-MS (Method 7): Rₜ=1.94 min; MS (ESIpos): m/z=290 [M+H]⁺

### Intermediate 233

### Ethyl 2-(3,4,5-trifluorophenyl)-2H-1,2,3-triazole-4-carboxylate

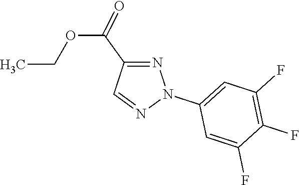 [SMILES: CCOC(=O)c1cnn(-c2cc(F)c(F)c(F)c2)n1]

Ethyl (2E,3E)-3-(hydroxyimino)-2-[2-(3,4,5-trifluorophenyl)hydrazinylidene]propanoate (3.17 g, 79% purity, 8.66 mmol) was stirred for one hour in 44 ml of acetic acid anhydride at 60° C. The reaction was then treated with water and extracted with ethyl acetate. The organic layer was dried over sodium sulfate, filtered and evaporated. The residue purified by chromatography on silica gel (100 g Ultra Snap Cartridge Biotage; eluent ethyl acetate/cyclohexane, elution gradient 5%→25%; flow: 100 ml/min). Samples containing the desired product were united, the solvents were evaporated and the residue was dried in vacuum. 1.96 g (100% purity, 83% yield) of the title compound were obtained.

LC-MS (Method 8): Rₜ=1.10 min; MS (ESIpos): m/z=272 [M+H]⁺

¹H-NMR (500 MHz, DMSO-d₆) δ[ppm]: 1.306 (0.66), 1.321 (0.59), 1.334 (7.89), 1.348 (16.00), 1.362 (7.67), 4.365 (0.42), 4.377 (2.69), 4.391 (7.71), 4.405 (7.48), 4.419 (2.36), 8.021 (2.92), 8.034 (3.42), 8.038 (3.42), 8.051 (2.95), 8.675 (9.00).

### Intermediate 234

### 2-(3,4,5-Trifluorophenyl)-2H-1,2,3-triazole-4-carboxylic acid

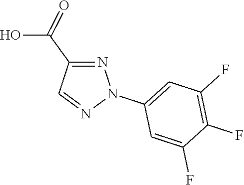 [SMILES: O=C(O)c1cnn(-c2cc(F)c(F)c(F)c2)n1]

ethyl 2-(3,4,5-trifluorophenyl)-2H-1,2,3-triazole-4-carboxylate (1.33 g, 4.91 mmol) was dissolved in 30 ml of THF, treated with lithium hydroxide solution (4.9 ml, 1.0 M, 4.9 mmol) and the reaction mixture was stirred for 2 h at RT. After acidification with 2 N hydrochloric acid (pH2-3), the resulting mixture was concentrated in vacuo, diluted with ethylacetate and a small amount of water. After phase separation, the organic phase was dried, concentrated in vacuo and 1.12 g (97% purity, 91% yield) of the desired compound were obtained.

LC-MS (Method 7): Rₜ=1.44 min; MS (ESIneg): m/z=242 [M−H]⁻

¹H-NMR (500 MHz, DMSO-d6) δ [ppm]: 0.007 (0.64), 3.307 (1.28), 7.999 (4.83), 8.011 (5.61), 8.016 (5.37), 8.028 (4.75), 8.580 (16.00).

### Intermediate 235

### tert-butyl [2-(1-methyl-1H-pyrazol-5-yl)-2-oxoethyl]carbamate

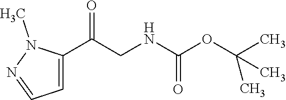 [SMILES: Cn1nccc1C(=O)CNC(=O)OC(C)(C)C]

To a solution of 2-amino-1-(2-methylpyrazol-3-yl)ethanone hydrochloride (3.00 g, 17.1 mmol) in dichloromethane (77 ml) was added di-tert-butyl dicarbonate (4.10 g, 18.8 mmol) and triethylamine (7.1 ml, 51 mmol). After 2 h of stirring at RT, the reaction mixture was diluted with dichloromethane and washed with water. The organic phase was dried, concentrated in vacuo and 4.40 g (91% purity, 98% yield) of the title compound was used without further purification.

LC-MS (Method 7): Rₜ=1.47 min; MS (ESIpos): m/z=240 [M+H]⁺

¹H-NMR (400 MHz, DMSO-d6) δ [ppm]: 0.943 (0.60), 1.111 (3.12), 1.280 (0.80), 1.366 (0.51), 1.372 (0.47), 1.394 (16.00), 4.046 (6.83), 4.256 (1.81), 4.271 (1.81), 7.143 (0.56), 7.202 (1.19), 7.207 (1.20), 7.553 (1.30), 7.558 (1.32).

### Intermediate 236

### rac-tert-butyl {[4-(1-methyl-1H-pyrazol-5-yl)-2,5-dioxoimidazolidin-4-yl]methyl}carbamate

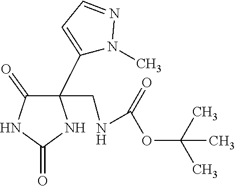 [SMILES: Cn1nccc1C1(CNC(=O)OC(C)(C)C)NC(=O)NC1=O]

The reaction was performed in 2 batches.

A) To a solution of tert-butyl [2-(1-methyl-1H-pyrazol-5-yl)-2-oxoethyl]carbamate (2.00 g, 8.36 mmol) in methanol (15.3 ml) was added potassium cyanide (2.18 g, 33.4 mmol) and ammonium carbonate (3.21 g, 33.4 mmol) at RT. The reaction mixture was heated up to 80° C. overnight into a sealed pressure flask. After filtration at RT, the filtrate was concentrated in vacuo and the crude was suspended in methanol. The suspension was filtered off and the resulting filtrate was concentrated in vacuo. (Batch 1)

B) To a solution of tert-butyl [2-(1-methyl-1H-pyrazol-5-yl)-2-oxoethyl]carbamate (2.00 g, 8.36 mmol) in a mixture of Ethanol (15 ml) and water (6 ml) was added potassium cyanide (2.18 g, 33.4 mmol) and ammonium carbonate (3.21 g, 33.4 mmol) at RT. The reaction mixture was heated up to 40° C. overnight into a sealed pressure flask. After filtration at RT, the resulting filtrate was concentrated in vacuo. (Batch 2)

The combined crude product (batch1+2) was purified by preparative HPLC (sample preparation: 15 g dissolved in a mixture of methanol, water and acetonitrile; column: XBridge C18 5 μm, 100×30 mm; eluent:water→acetonitrile/water 80:20+1% ammonia solution→acetonitrile; flow rate: 80 ml/min; temperature: 40° C.; UV detection: 220 nm). After lyophilisation, 1.8 g of the desired product was obtained and used without further purification.

LC-MS (Method 9): Rₜ=0.36 min; MS (ESIpos): m/z=310 [M+H]⁺

¹H-NMR (400 MHz, DMSO-d₆) δ[ppm]: −0.008 (0.43), 0.008 (0.54), 1.359 (16.00), 1.754 (0.51), 3.524 (0.41), 3.540 (0.41), 3.693 (4.71), 6.230 (0.96), 7.250 (0.96).

The title compound can also be synthesized via the procedure described in J: Med. Chem. 2014, 57, 10476-10485

### Intermediate 237

### rac-5-(aminomethyl)-5-(1-methyl-1H-pyrazol-5-yl)imidazolidine-2,4-dione hydrochloride

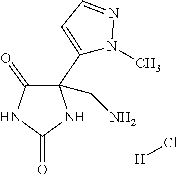

To a solution of rac-tert-butyl {[4-(1-methyl-1H-pyrazol-5-yl)-2,5-dioxoimidazolidin-4-yl]methyl}carbamate (520 mg, 1.68 mmol) in dichloromethane (14.5 ml) were added 2.1 ml (8.4 mmol) of 4M hydrochloric acid in dioxane at RT. The reaction mixture was stirred overnight and concentrated in vacuo. 400 mg (100% purity, 97% yield) of the title compound was used without further purification.

LC-MS (Method 9): Rₜ=0.22 min; MS (ESIneg): m/z=208 [M−HCl−H]⁻

¹H-NMR (600 MHz, DMSO-d6) δ [ppm]: 1.767 (3.19), 2.119 (1.47), 2.390 (0.50), 3.166 (0.76), 3.463 (1.91), 3.473 (2.29), 3.485 (2.90), 3.494 (2.58), 3.590 (2.66), 3.598 (2.94), 3.612 (2.25), 3.620 (1.90), 3.725 (0.42), 4.091 (0.50), 5.692 (1.73), 6.501 (15.58), 6.504 (16.00), 7.358 (0.46), 7.425 (15.98), 7.428 (15.92), 8.608 (10.32), 8.873 (12.22), 11.515 (9.95).

The title compound can also be synthesized via the procedure described in J: Med. Chem. 2014, 57, 10476-10485

### Intermediate 238

### ethyl 5-[1-(difluoromethyl)-1H-pyrazol-5-yl]-1,3-oxazole-4-carboxylate

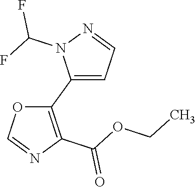 [SMILES: CCOC(=O)c1ncoc1-c1ccnn1C(F)F]

1-(difluoromethyl)-1H-pyrazole-5-carboxylic acid (2.60 g, 16.0 mmol) dissolved in 25 ml of THF was treated with 1,1,-carbonyl-diimidazole (3.12 g, 19.2 mmol) and stirred at room temperature. After 2 h ethyl isocyanoacetate (1.9 ml, 18 mmol), dissolved in 25 ml of THF, and a solution of lithium bis(trimethylsilyl)amide in THF (16 ml, 1.0 M, 16 mmol) were added at 0° C. The mixture was allowed to warm to room temperature, stirred overnight and then concentrated in vacuo The residue was taken up with ethyl acetate and washed with water. The organic layer was dried over sodium sulfate, filtered, Isolute® was added and the solvent was evaporated on a rotary evaporator. The crude product was purified by column chromatography (100 g Ultra Snap Cartridge Biotage; eluent ethyl acetate/cyclohexane, elution gradient 10%→65%). Samples containing the desired product were united, the solvents were evaporated and the residue was dried in vacuum. 1.60 g (100% purity, 39% yield) of the title compound was obtained.

LC-MS (Method 7): Rₜ=1.43 min; MS (ESIpos): m/z=258 [M+H]⁺

¹H-NMR (400 MHz, DMSO-d6) δ [ppm]: 1.173 (7.68), 1.191 (16.00), 1.209 (7.93), 4.212 (2.57), 4.230 (7.90), 4.247 (7.83), 4.265 (2.52), 7.086 (4.50), 7.090 (4.56), 7.709 (2.14), 7.851 (4.21), 7.994 (2.25), 8.000 (4.40), 8.004 (4.29), 8.759 (6.93).

### Intermediate 239

### 2-amino-1-[1-(difluoromethyl)-1H-pyrazol-5-yl]ethanone hydrochloride

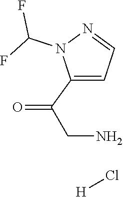

ethyl 5-[1-(difluoromethyl)-1H-pyrazol-5-yl]-1,3-oxazole-4-carboxylate (1.60 g, 6.22 mmol) was taken up in 35 ml of 6 N hydrochloric acid and stirred at 100° C. After 2 h the resulting mixture was concentrated in vacuo and the residue was treated with DCM and a small amount of methanol. The precipitate was filtered off and dried in vacuo. 1.10 g (84% yield) of the title compound was obtained.

LC-MS (Method 9): Rₜ=0.74 min; MS (ESIpos): m/z=176 [M−HCl+H]⁺

¹H-NMR (400 MHz, DMSO-d6) δ [ppm]: 1.091 (0.50), 1.109 (1.11), 1.127 (0.59), 2.328 (0.75), 2.367 (1.14), 2.670 (0.78), 2.711 (1.17), 4.421 (0.53), 4.492 (13.60), 7.583 (16.00), 7.587 (15.25), 7.957 (6.86), 8.047 (13.41), 8.102 (13.69), 8.248 (6.77), 8.418 (8.00).

### Intermediate 240

### tert-butyl {2-[1-(difluoromethyl)-1H-pyrazol-5-yl]-2-oxoethyl}carbamate

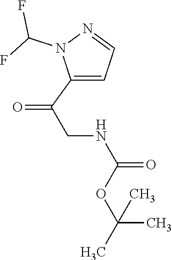 [SMILES: CC(C)(C)OC(=O)NCC(=O)c1ccnn1C(F)F]

To a solution of 2-amino-1-[1-(difluoromethyl)-1H-pyrazol-5-yl]ethanone hydrochloride (1.10 g, 5.20 mmol) in dichloromethane (23 ml) was added di-tert-butyl dicarbonate (1.25 g, 5.72 mmol) and triethylamine (3.6 ml, 26 mmol). After 2 h of stirring at RT, the reaction mixture was diluted with dichloromethane and washed with water. The organic phase was dried, concentrated in vacuo and 1.44 g of the title compound was used without further purification.

LC-MS (Method 7): Rₜ=1.58 min; MS (ESIpos): m/z=276 [M+H]⁺

### Intermediate 241

### rac-tert-butyl ({4-[1-(difluoromethyl)-1H-pyrazol-5-yl]-2,5-dioxoimidazolidin-4-yl}methyl)carbamate

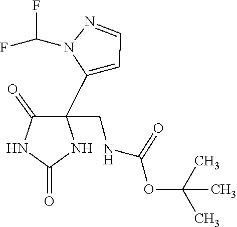 [SMILES: CC(C)(C)OC(=O)NCC1(c2ccnn2C(F)F)NC(=O)NC1=O]

To a solution of tert-butyl {2-[1-(difluoromethyl)-1H-pyrazol-5-yl]-2-oxoethyl}carbamate (1.44 g, 5.23 mmol) in methanol (9.6 ml) was added potassium cyanide (1.36 g, 20.9 mmol) and ammonium carbonate (2.01 g, 20.9 mmol) at RT. The reaction mixture was stirred for 2 days at 60° C. into a sealed pressure flask. After filtration at RT, the resulting filtrate was concentrated in vacuo. The residue was purified by preparative HPLC (sample preparation: 3.9 g dissolved in a mixture of methanol, water, acetonitrile and ammonia; column: XBridge C18 5 μm, 100×30 mm; eluent:water→acetonitrile/water 80:20+1% ammonia solution→acetonitrile; flow rate: 80 ml/min; temperature: 40° C.; UV detection: 220 nm). 699 mg of the desired product was obtained and used without further purification.

LC-MS (Method 7): Rₜ=1.08 min; MS (ESIneg): m/z=344 [M−H]⁻

### Intermediate 242

### rac-5-(aminomethyl)-5-[1-(difluoromethyl)-1H-pyrazol-5-yl]imidazolidine-2,4-dione hydrochloride

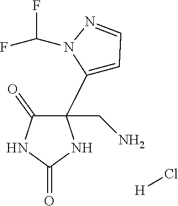 [SMILES: NCC1(c2ccnn2C(F)F)NC(=O)NC1=O ; Cl]

To a solution of rac-tert-butyl ({4-[1-(difluoromethyl)-1H-pyrazol-5-yl]-2,5-dioxoimidazolidin-4-yl}methyl)carbamate (340 mg, 985 μmol) in dichloromethane (8.5 ml) were added 1.2 ml (4.9 mmol) of 4M hydrochloric acid in dioxane at RT. The reaction mixture was stirred for 2 h and concentrated in vacuo. 280 mg (99% purity, 100% yield) of the title compound was used without further purification.

LC-MS (Method 9): Rₜ=0.22 min; MS (ESIpos): m/z=246 [M−HCl+H]⁺

¹H-NMR (400 MHz, DMSO-d₆) δ[ppm]: −0.149 (0.44), −0.008 (3.88), 0.008 (3.67), 0.146 (0.44), 1.110 (0.42), 1.596 (0.84), 1.754 (5.24), 2.111 (2.45), 2.324 (0.50), 2.329 (0.71), 2.333 (0.50), 2.367 (0.75), 2.524 (2.73), 2.666 (0.52), 2.671 (0.71), 2.675 (0.52), 2.711 (0.73), 3.457 (5.03), 3.491 (6.16), 3.568 (2.20), 3.689 (5.72), 3.723 (4.42), 5.756 (1.11), 6.811 (15.81), 6.816 (16.00), 7.820 (4.47), 7.835 (12.96), 7.839 (12.85), 7.960 (4.43), 7.969 (4.22), 8.109 (3.69), 8.504 (6.88), 8.825 (4.30), 11.497 (3.56).

### Intermediate 243

### ethyl 5-[5-(trifluoromethyl)-1,3-thiazol-4-yl]-1,3-oxazole-4-carboxylate

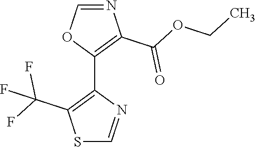 [SMILES: CCOC(=O)c1ncoc1-c1ncsc1C(F)(F)F]

5-(trifluoromethyl)-1,3-thiazole-4-carboxylic acid (2.00 g, 10.1 mmol) dissolved in 15 ml of THF was treated with 1,1,-carbonyl-diimidazole (1.97 g, 12.2 mmol) and stirred at room temperature. After 1 h ethyl isocyanoacetate (1.2 ml, 11 mmol), dissolved in 15 ml of THF, and a solution of lithium bis(trimethylsilyl)amide in THF (10 ml, 1.0 M, 10 mmol) were added at 0° C. The mixture was allowed to warm to room temperature, stirred over 2 days and then concentrated in vacuo. The residue was taken up with ethyl acetate and washed with water. The organic layer was dried and concentrated in vacuo. The crude product was purified by column chromatography (100 g Ultra Snap Cartridge Biotage; eluent ethyl acetate/cyclohexane, elution gradient 8%→85%). Samples containing the desired product were united, the solvents were evaporated and the residue was dried in vacuum. 1.43 g (48% yield) of the title compound was obtained.

LC-MS (Method 7): Rₜ=1.62 min; MS (ESIpos): m/z=293 [M+H]⁺

¹H-NMR (400 MHz, DMSO-d6) δ [ppm]: 1.086 (7.76), 1.104 (16.00), 1.122 (8.00), 4.148 (2.68), 4.166 (8.12), 4.184 (8.05), 4.201 (2.61), 8.780 (5.87), 9.570 (4.98).

### Intermediate 244

### 2-amino-1-[5-(trifluoromethyl)-1,3-thiazol-4-yl]ethanone hydrochloride

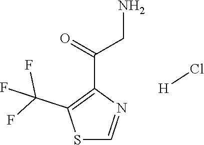 [SMILES: NCC(=O)c1ncsc1C(F)(F)F ; Cl]

ethyl 5-[5-(trifluoromethyl)-1,3-thiazol-4-yl]-1,3-oxazole-4-carboxylate (1.43 g, 4.89 mmol) was taken up in 200 ml of 6 N hydrochloric acid and stirred for 2 h at 100° C. After further stirring overnight at room temperature, the resulting mixture was concentrated in vacuo. 1.25 g (95% purity, 98% yield) of the title compound was obtained.

LC-MS (Method 9): Rₜ=1.00 min; MS (ESIpos): m/z=211 [M−HCl+H]⁺

¹H-NMR (400 MHz, DMSO-d6) δ [ppm]: −0.008 (1.13), 0.008 (1.18), 4.571 (16.00), 8.411 (4.83), 9.517 (12.18).

### Intermediate 245

### tert-butyl {2-oxo-2-[5-(trifluoromethyl)-1,3-thiazol-4-yl]ethyl}carbamate

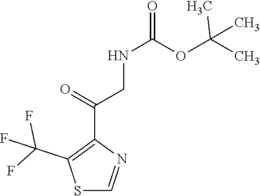 [SMILES: CC(C)(C)OC(=O)NCC(=O)c1ncsc1C(F)(F)F]

To a solution of 2-amino-1-[5-(trifluoromethyl)-1,3-thiazol-4-yl]ethanone hydrochloride (1.25 g, 5.07 mmol) in dichloromethane (23 ml) was added di-tert-butyl dicarbonate (1.22 g, 5.57 mmol) and triethylamine (3.5 ml, 25 mmol). After 2 h of stirring at RT, the reaction mixture was diluted with dichloromethane and washed with water. The organic phase was dried, concentrated in vacuo and 1.66 g of the title compound was used without further purification.

LC-MS (Method 9): Rₜ=1.58 min; MS (ESIpos): m/z=310 [M−H]⁺

### Intermediate 246

### rac-tert-butyl ({2,5-dioxo-4-[5-(trifluoromethyl)-1,3-thiazol-4-yl]imidazolidin-4-yl}methyl)carbamate

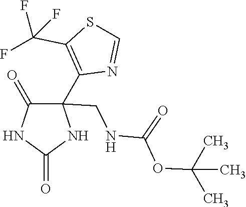 [SMILES: CC(C)(C)OC(=O)NCC1(c2ncsc2C(F)(F)F)NC(=O)NC1=O]

To a solution of tert-butyl {2-oxo-2-[5-(trifluoromethyl)-1,3-thiazol-4-yl]ethyl}carbamate (800 mg, 2.58 mmol) in methanol (4.6 ml) was added potassium cyanide (672 mg, 10.3 mmol) and ammonium carbonate (991 mg, 10.3 mmol) at RT. The reaction mixture was stirred overnight at 60° C. into a sealed pressure flask. After filtration at RT, the resulting filtrate was concentrated in vacuo. The residue was purified by preparative HPLC (sample preparation: 473 mg dissolved in a mixture of methanol, water, acetonitrile and ammonia; column: XBridge C18 5 μm, 100×30 mm; eluent:water→acetonitrile/water 80:20+1% ammonia solution→acetonitrile; flow rate: 80 ml/min; temperature: 40° C.; UV detection: 220 nm). 227 mg (100% purity, 23% yield) of the desired product was obtained and used without further purification.

LC-MS (Method 9): Rₜ=0.76 min; MS (ESIneg): m/z=379 [M−H]⁻

### Intermediate 247

### rac-5-(aminomethyl)-5-[5-(trifluoromethyl)-1,3-thiazol-4-yl]imidazolidine-2,4-dione hydrochloride

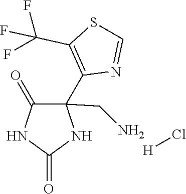

To a solution of rac-tert-butyl ({2,5-dioxo-4-[5-(trifluoromethyl)-1,3-thiazol-4-yl]imidazolidin-4-yl}methyl)carbamate (485 mg, 1.28 mmol) in dichloromethane (20 ml) were added 1.2 ml (1.6 ml, 4.0 M, 6.4 mmol) of 4M hydrochloric acid in dioxane at RT. The reaction mixture was stirred overnight and concentrated in vacuo. 413 mg (95% purity, 97% yield) of the title compound was used without further purification.

LC-MS (Method 9): Rₜ=0.29 min; MS (ESIpos): m/z=281 [M−HCl+H]⁺

¹H-NMR (400 MHz, DMSO-d6) δ [ppm]: −0.008 (0.97), 0.008 (0.91), 1.754 (10.12), 2.108 (3.70), 2.367 (0.43), 2.519 (1.81), 2.524 (1.46), 2.558 (0.54), 2.560 (0.45), 2.710 (0.41), 3.167 (16.00), 3.708 (3.29), 3.741 (2.54), 8.321 (2.94), 8.628 (1.96), 9.440 (7.56), 11.450 (3.19).

### Intermediate 248

### Ethyl 5-(1-ethyl-1H-pyrazol-5-yl)-1,3-oxazole-4-carboxylate

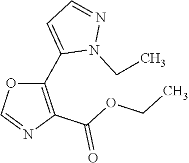 [SMILES: CCOC(=O)c1ncoc1-c1ccnn1CC]

A solution of 1-ethyl-1H-pyrazole-5-carboxylic acid (1.00 g, 7.14 mmol) and 1,1′-carbonyldiimidazole (1.39 g, 8.56 mmol) in 10 ml of THF was stirred for 2 h at room temperature. After that time, a solution of ethyl isocyanoacetate (860 μl, 7.8 mmol) in 10 ml of THF and a solution of lithium bis(trimethylsilyl)amide in THF (7.1 ml, 1.0 M, 7.1 mmol) were added dropwise at 0° C. When the addition was complete the resulting mixture was stirred at room temperature for 2 h. The reaction was concentrated in vacuo and extracted with ethyl acetate. The combined organic layers were dried over sodium sulfate, filtered and concentrated. The crude product was purified by column chromatography (Machine: Biotage® Isolera One; column: Biotage® SNAP Ultra 50 g; gradient: Cy/EE-gradient, 16% EE-100% EE; flow: 100 ml/min). Product containing samples were united and the solvents were removed on a rotary evaporator. 1.30 g (95% purity, 74% yield) of the title compound were obtained.

LC-MS (Method 7): Rₜ=1.23 min; MS (ESIpos): m/z=236 [M+H]⁺

### Intermediate 249

### 2-amino-1-(1-ethyl-1H-pyrazol-5-yl)ethan-1-one-hydrogen chloride (1/1)

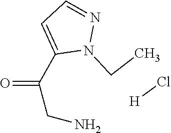

Ethyl 5-(1-ethyl-1H-pyrazol-5-yl)-1,3-oxazole-4-carboxylate (1.30 g, 5.53 mmol) were taken up in 29 ml of 6 N hydrochloric acid and stirred at 100° C. for 2 h. The solvent was removed on a rotary evaporator and the residue was taken up in DCM/MeOH 20:1. The solvent was removed an the residue was dried in vacuo. 1.19 g (114% yield) of the title compound were obtained.

### Intermediate 250

### tert-butyl [2-(1-ethyl-1H-pyrazol-5-yl)-2-oxoethyl]carbamate

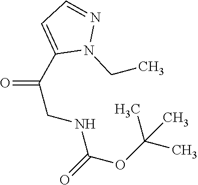 [SMILES: CCn1nccc1C(=O)CNC(=O)OC(C)(C)C]

2-amino-1-(1-ethyl-1H-pyrazol-5-yl)ethan-1-one-hydrogen chloride (1/1) (1.10 g, 5.80 mmol) dissolved in 23 ml of dichloromethane were treated with di-tert-butyl dicarbonate (1.5 ml, 6.4 mmol) and triethylamine (2.4 ml, 17 mmol). The mixture was stirred at room temperature for 2 h. The solvent was removed on a rotary evaporator. The residue was taken up with ethyl acetate and washed with water and saturated aqueous sodium chloride solution. The organic layer was dried over sodium sulfate, filtered and evaporated on a rotary evaporator. The crude product was purified by column chromatography (Machine: Biotage® Isolera One; column: Biotage® SNAP Ultra 25 g; gradient: Cy/EE-gradient, 12% EE-100% EE; flow: 75 ml/min). Product containing samples were united and the solvents were removed on a rotary evaporator. 1.07 g (100% purity, 73% yield) of the title compound were obtained.

LC-MS (Method 9): Rₜ=1.40 min; MS (ESIpos): m/z=254 [M+H]⁺

### Intermediate 251

### rac-tert-butyl {[4-(1-ethyl-1H-pyrazol-5-yl)-2,5-dioxoimidazolidin-4-yl]methyl}carbamate

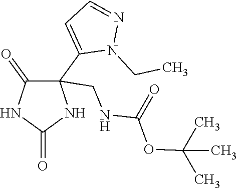 [SMILES: CCn1nccc1C1(CNC(=O)OC(C)(C)C)NC(=O)NC1=O]

In a microwave vial tert-butyl [2-(1-ethyl-1H-pyrazol-5-yl)-2-oxoethyl]carbamate (1.00 g, 3.95 mmol) was dissolved in 8 ml of methanol. Potassium cyanide (1.03 g, 15.8 mmol) and ammonium carbonate (1.52 g, 15.8 mmol) were added. The vial was sealed and the mixture was stirred at 50° C. for 48 h. The mixture was filtered and the filtrate was purified by preparative HPLC (Column: Chromatorex C18 10 μm 250×30 mm; eluent A=water, B=acetonitrile; gradient: 0.0 min 30% B; 4.5 min 50% B; 11.5 min 70% B; 12 min 100% B; 14.75 min 30% B; flow: 50 ml/min; 0.1% formic acid). Product containing samples were united and the solvents were removed on a rotary evaporator. 137 mg (100% purity, 11% yield) of the title compound were obtained.

LC-MS (Method 7): Rₜ=1.02 min; MS (ESIpos): m/z=324 [M+H]⁺

### Intermediate 252

### rac-5-(aminomethyl)-5-(1-ethyl-1H-pyrazol-5-yl)imidazolidine-2,4-dione-hydrogen chloride

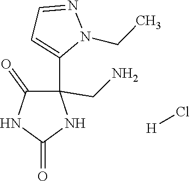 [SMILES: CCn1nccc1C1(CN)NC(=O)NC1=O ; Cl]

tert-butyl {[4-(1-ethyl-1H-pyrazol-5-yl)-2,5-dioxoimidazolidin-4-yl]methyl}carbamate (137 mg, 424 μmol) was dissolved in 2 ml of dichloromethane. 4 M hydrochloric acid in 1,4-dioxane (530 μl, 4.0 M, 2.1 mmol) was added and the mixture was stirred overnight. The solvent was removed on a rotary evaporator and the residue was dried in vacuo. 142 mg (90% purity, 116% yield) of the title compound were obtained.

LC-MS (Method 9): Rₜ=0.22 min; MS (ESIpos): m/z=224 [M−HCl+H]⁺

### Intermediate 253

### Ethyl 5-(4-methyl-1,3-thiazol-5-yl)-1,3-oxazole-4-carboxylate

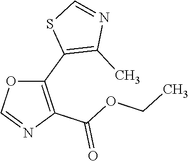 [SMILES: CCOC(=O)c1ncoc1-c1scnc1C]

A solution of 4-methyl-1,3-thiazole-5-carboxylic acid (5.00 g, 34.9 mmol) and 1,1′-carbonyldiimidazole (6.80 g, 41.9 mmol) in 50 ml of THF was stirred for 2 h at room temperature. After that time, a solution of ethyl isocyanoacetate (4.2 ml, 38 mmol) in 50 ml of THF and a solution of lithium bis(trimethylsilyl)amide in THF (35 ml, 1.0 M, 35 mmol) were added dropwise at 0° C. When the addition was complete the resulting mixture was stirred at room temperature over night. The reaction was concentrated in vacuo and extracted with ethyl acetate. The combined organic layers were dried over sodium sulfate, filtered and concentrated. The crude product was purified by column chromatography (Machine: Biotage® Isolera One; column: Biotage® SNAP Ultra 100 g; gradient: Cy/EE-gradient, 12% EE-100% EE; flow: 100 ml/min).

Product containing samples were united and the solvents were removed on a rotary evaporator. 5.76 g (100% purity, 69% yield) of the title compound were obtained.

LC-MS (Method 7): Rₜ=1.15 min; MS (ESIpos): m/z=239 [M+H]⁺

### Intermediate 254

### 2-amino-1-(4-methyl-1,3-thiazol-5-yl)ethan-1-one-hydrogen chloride

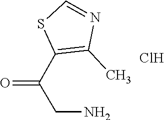

Ethyl 5-(4-methyl-1,3-thiazol-5-yl)-1,3-oxazole-4-carboxylate (5.76 g, 24.2 mmol) were taken up in 130 ml of 6 N hydrochloric acid and stirred at 100° C. for 1 h. The solvent was removed on a rotary evaporator and the residue was taken up in DCM/MeOH 20:1. The precipitate was filtered off, washed with dichloromethane and dried in vacuo. 4.79 g (97% purity, 100% yield) of the title compound were obtained.

LC-MS (Method 9): Rₜ=0.62 min; MS (ESIpos): m/z=157 [M−HCl+H]⁺

### Intermediate 255

### tert-butyl [2-(4-methyl-1,3-thiazol-5-yl)-2-oxoethyl]carbamate

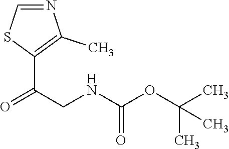 [SMILES: Cc1ncsc1C(=O)CNC(=O)OC(C)(C)C]

2-amino-1-(4-methyl-1,3-thiazol-5-yl)ethan-1-one-hydrogen chloride (4.79 g, 24.9 mmol) dissolved in 100 ml of dichloromethane were treated with di-tert-butyl dicarbonate (6.3 ml, 27 mmol) and triethylamine (10 ml, 75 mmol). The mixture was stirred at room temperature for 4 h. The solvent was removed on a rotary evaporator. The residue was taken up with ethyl acetate and washed with water and saturated aqueous sodium chloride solution. The organic layer was dried over sodium sulfate, filtered and evaporated on a rotary evaporator. 4.98 g (100% purity, 78% yield) of the title compound were obtained.

LC-MS (Method 7): Rₜ=1.50 min; MS (ESIpos): m/z=257 [M+H]⁺

### Intermediate 256

### rac-tert-butyl {[4-(4-methyl-1,3-thiazol-5-yl)-2,5-dioxoimidazolidin-4-yl]methyl}carbamate

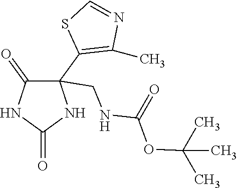 [SMILES: Cc1ncsc1C1(CNC(=O)OC(C)(C)C)NC(=O)NC1=O]

In a microwave vial tert-butyl [2-(4-methyl-1,3-thiazol-5-yl)-2-oxoethyl]carbamate (4.98 g, 19.4 mmol) was dissolved in 30 ml of methanol. Potassium cyanide (5.06 g, 77.7 mmol) and ammonium carbonate (7.47 g, 77.7 mmol) were added. The vial was sealed and the mixture was stirred at 60° C. for 3 d. The crude product was purified by column chromatography (Machine: Biotage® Isolera One; column: Biotage® SNAP Ultra 100 g; gradient: DCM/MeOH-gradient, 2% MeOH-20% MeOH, flow: 100 ml/min). Product containing samples were united and the solvents were removed on a rotary evaporator. 3.00 g (100% purity, 47% yield) of the title compound were obtained.

LC-MS (Method 7): Rₜ=1.11 min; MS (ESIpos): m/z=327 [M+H]⁺

### Intermediate 257

### rac-5-(aminomethyl)-5-(4-methyl-1,3-thiazol-5-yl)imidazolidine-2,4-dione-hydrogen chloride

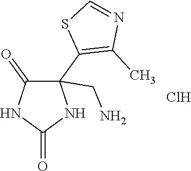

rac-tert-butyl {[4-(4-methyl-1,3-thiazol-5-yl)-2,5-dioxoimidazolidin-4-yl]methyl}carbamate (200 mg, 613 μmol) was dissolved in 3 ml of dichloromethane. 4 M hydrochloric acid in 1,4-dioxane (766 μl, 4.0 M, 3.1 mmol) was added and the mixture was stirred for 2 h. The precipitate was filtered off, washed with dichloromethane and dried in vacuo. 225 mg (95% purity, 133% yield) of the title compound were obtained.

LC-MS (Method 9): Rₜ=0.23 min; MS (ESIpos): m/z=227 [M−HCl+H]⁺

### Intermediate 258

### Ethyl (2E)-3-oxo-2-[2-(1,2-thiazol-4-yl)hydrazinylidene]propanoate

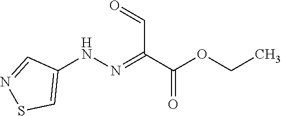

1,2-thiazol-4-amine-hydrogen chloride (250 mg, 1.83 mmol) was dissolved in 3 ml of water and 400 μl of concentrated hydrochloric acid. The mixture was cooled to 0° C. and a solution of sodium nitrite (126 mg, 1.83 mmol) in 3 ml of water was added dropwise. After stirring for 5 minutes this solution was added dropwise to a solution of ethyl-(2E)-3-(dimethylamino)acrylate (290 μl, 2.0 mmol) and potassium acetate (269 mg, 2.75 mmol) in 3 ml of ethanol at 0° C. The mixture was stirred at room temperature for 30 min before being diluted with water. The precipitate was filtered off, washed with water and dried in vacuo. 170 mg (100% purity, 41% yield) of the title compound were obtained.

LC-MS (Method 7): Rₜ=1.33 min; MS (ESIpos): m/z=228 [M+H]⁺

### Intermediate 259

### Ethyl (2E,3E)-3-(hydroxyimino)-2-[2-(1,2-thiazol-4-yl)hydrazinylidene]propanoate

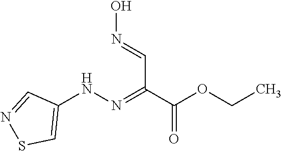 [SMILES: CCOC(=O)C(/C=N/O)=N/Nc1cnsc1]

Ethyl (2E)-3-oxo-2-[2-(1,2-thiazol-4-yl)hydrazinylidene]propanoate (170 mg, 748 μmol), hydroxylamine hydrochloride (62.4 mg, 898 μmol) and potassium acetate (184 mg, 1.87 mmol) were dissolved in 7 ml of ethanol and the mixture was stirred over night at room temperature. The reaction mixture was diluted with water. The precipitate was filtered off, washed with water and dried in vacuo. 80.0 mg (100% purity, 44% yield) of the title compound were obtained.

LC-MS (Method 7): Rₜ=1.45 min; MS (ESIpos): m/z=243 [M+H]⁺

### Intermediate 260

### Ethyl 2-(1,2-thiazol-4-yl)-2H-1,2,3-triazole-4-carboxylate

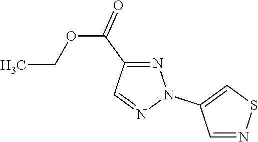

Ethyl (2E,3E)-3-(hydroxyimino)-2-[2-(1,2-thiazol-4-yl)hydrazinylidene]propanoate (80.0 mg, 330 μmol) was stirred for 1.5 h in 2 ml of acetic acid anhydride at 60° C. The mixture was purified by preparative HPLC (Column: Chromatorex C18 10 μm 250×30 mm; eluent A=water, B=acetonitrile; gradient: 0.0 min 30% B; 4.5 min 50% B; 11.5 min 70% B; 12 min 100% B; 14.75 min 30% B; flow: 50 ml/min; 0.1% formic acid). Product containing samples were united and the solvents were removed on a rotary evaporator. 60.0 mg (100% purity, 81% yield) of the title compound were obtained.

LC-MS (Method 8): Rₜ=0.78 min; MS (ESIpos): m/z=225 [M+H]⁺

### Intermediate 261

### 2-(1,2-thiazol-4-yl)-2H-1,2,3-triazole-4-carboxylic acid

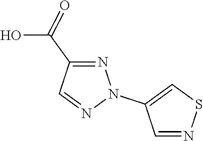 [SMILES: O=C(O)c1cnn(-c2cnsc2)n1]

Ethyl 2-(1,2-thiazol-4-yl)-2H-1,2,3-triazole-4-carboxylate (60.0 mg, 268 μmol) was dissolved in 2.5 ml of THF, treated with lithium hydroxide solution (270 μl, 1.0 M, 270 μmol) and the mixture was stirred for 1 h at room temperature. The solvent was removed on a rotary evaporator. Water was added an the mixture was acidified with 2 N hydrochloric acid. The precipitate was filtered off, washed with water an dried in vacuo. 45.0 mg (100% purity, 86% yield) of the title compound were obtained

LC-MS (Method 8): Rₜ=0.41 min; MS (ESIpos): m/z=197 [M+H]⁺

### Intermediate 262

### Ethyl 5-[1-(2,2,2-trifluoroethyl)-1H-pyrazol-5-yl]-1,3-oxazole-4-carboxylate

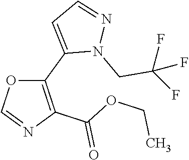 [SMILES: CCOC(=O)c1ncoc1-c1ccnn1CC(F)(F)F]

A solution of 1-(2,2,2-trifluoroethyl)-1H-pyrazole-5-carboxylic acid (1.00 g, 5.15 mmol) and 1,1′-carbonyldiimidazole (1.00 g, 6.18 mmol) in 11 ml of THF was stirred for 2 h at room temperature. After that time, a solution of ethyl isocyanoacetate (620 μl, 5.7 mmol) in 11 ml of THF and a solution of lithium bis(trimethylsilyl)amide in THF (5.2 ml, 1.0 M, 5.2 mmol) were added dropwise at 0° C. When the addition was complete the resulting mixture was stirred at room temperature for 1 h. The reaction was concentrated in vacuo and extracted with ethyl acetate. The combined organic layers were dried over sodium sulfate, filtered and concentrated. The crude product was purified by column chromatography (Machine: Biotage® Isolera One; column: Biotage® SNAP

Ultra 25 g; gradient: Cy/EE-gradient, 12% EE-100% EE; flow: 75 ml/min). Product containing samples were united and the solvents were removed on a rotary evaporator. 1.16 g (100% purity, 78% yield) of the title compound were obtained.

LC-MS (Method 7): Rₜ=1.43 min; MS (ESIpos): m/z=290 [M+H]⁺

### Intermediate 263

### 2-amino-1-[1-(2,2,2-trifluoroethyl)-1H-pyrazol-5-yl]ethan-1-one-hydrogen chloride

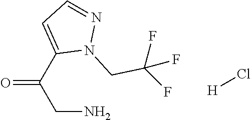

Ethyl 5-[1-(2,2,2-trifluoroethyl)-1H-pyrazol-5-yl]-1,3-oxazole-4-carboxylate (1.16 g, 4.01 mmol) were taken up in 20 ml of 6 N hydrochloric acid and stirred at 100° C. for 1 h. The solvent was removed on a rotary evaporator and the residue was taken up in DCM/MeOH 20:1. The solvent was removed and the residue was dried in vacuo. 1.07 g (100% purity, 110% yield of the title compound were obtained.

LC-MS (Method 9): Rₜ=0.95 min; MS (ESIpos): m/z=208 [M−HCl+H]⁺

### Intermediate 264

### tert-butyl {2-oxo-2-[1-(2,2,2-trifluoroethyl)-1H-pyrazol-5-yl]ethyl}carbamate

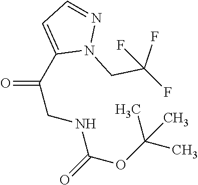 [SMILES: CC(C)(C)OC(=O)NCC(=O)c1ccnn1CC(F)(F)F]

2-amino-1-[1-(2,2,2-trifluoroethyl)-1H-pyrazol-5-yl]ethan-1-one-hydrogen chloride (1.07 g, 4.39 mmol) dissolved in 17 ml of dichloromethane were treated with di-tert-butyl dicarbonate (1.1 ml, 4.8 mmol) and triethylamine (1.8 ml, 13 mmol). The mixture was stirred at room temperature for 3 h. The solvent was removed on a rotary evaporator. The residue was taken up with ethyl acetate and washed with water and saturated aqueous sodium chloride solution. The organic layer was dried over sodium sulfate, filtered and evaporated on a rotary evaporator. 1.37 g (71% purity, 72% yield) of the title compound were obtained.

LC-MS (Method 9): Rₜ=1.52 min; MS (ESIpos): m/z=308 [M+H]⁺

### Intermediate 265

### rac-tert-butyl({2,5-dioxo-4-[1-(2,2,2-trifluoroethyl)-1H-pyrazol-5-yl]imidazolidin-4-yl}methyl)carbamate

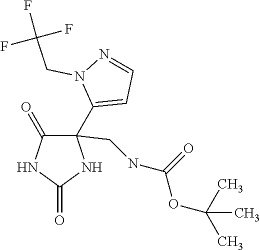 [SMILES: CC(C)(C)OC(=O)NCC1(c2ccnn2CC(F)(F)F)NC(=O)NC1=O]

In a microwave vial tert-butyl {2-oxo-2-[1-(2,2,2-trifluoroethyl)-1H-pyrazol-5-yl]ethyl}carbamate (689 mg, 2.24 mmol) was dissolved in 5 ml of methanol. Potassium cyanide (730 mg, 11.2 mmol) and ammonium carbonate (1.08 g, 11.2 mmol) were added. The vial was sealed and the mixture was stirred at 50° C. for 48 h. The mixture was filtered and the filtrate was purified by preparative HPLC (Column: Chromatorex C1810 μm 250×30 mm; eluent A=water, B=acetonitrile; gradient: 0.0 min 30% B; 4.5 min 50% B; 11.5 min 70% B; 12 min 100% B; 14.75 min 30% B; flow: 50 ml/min; 0.1% formic acid). Product containing samples were united and the solvents were removed on a rotary evaporator. 195 mg (100% purity, 23% yield) of the title compound were obtained.

LC-MS (Method 7): Rₜ=1.21 min; MS (ESIpos): m/z=378 [M+H]⁺

### Intermediate 266

### rac-5-(aminomethyl)-5-[1-(2,2,2-trifluoroethyl)-1H-pyrazol-5-yl]imidazolidine-2,4-dione hydrochloride

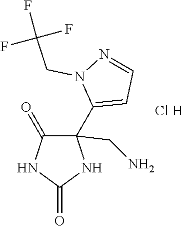

rac-tert-butyl({2,5-dioxo-4-[1-(2,2,2-trifluoroethyl)-1H-pyrazol-5-yl]imidazolidin-4-yl}methyl)carbamate (195 mg, 517 μmol) was dissolved in 2 ml of dichloromethane. 4 M hydrochloric acid in 1,4-dioxane (650 μl, 4.0 M, 2.6 mmol) was added and the mixture was stirred for 3 h. The solvent was removed on a rotary evaporator. The residue was taken up in dichloromethane, the solvent was removed and the residue was dried in vacuo. 155 mg (96% purity, 92% yield) of the title compound were obtained.

LC-MS (Method 9): Rₜ=0.23 min; MS (ESIpos): m/z=278 [M−HCl+H]⁺

### Intermediate 267

### 3,5-difluoro-2-hydrazinylpyridine

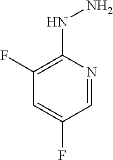

2,3,5-trifluoropyridine (670 μl, 7.5 mmol) were dissolved in hydrazine hydrate (3.7 ml, 75 mmol). The mixture was stirred at 120° C. for 1 h. The mixture was extracted with dichloromethane, the organic layer was dried over sodium sulfate, filtered and evaporated on a rotary evaporator. 403 mg (100% purity, 37% yield) of the title compound were obtained.

GC-MS (Method A): Rₜ=2.52 min; MS (EI-MS-POS): m/z=145 [M]⁺

### Intermediate 268

### N-{(1E,2Z)-2-[2-(3,5-difluoropyridin-2-yl)hydrazinylidene]propylidene}hydroxylamine

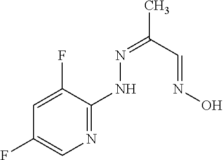

3,5-difluoro-2-hydrazinylpyridine (403 mg, 2.78 mmol) was dissolved in 12 ml of ethanol. (1E)-1-(hydroxyimino)propan-2-one (290 mg, 3.33 mmol) was added and the mixture was stirred at 80° C. After 2 h the solvent was removed on a rotary evaporator. The residue was taken up with pentane. The precipitate was filtered off, washed with pentane and dried in vacuo. 601 mg (38% purity, 38% yield) of the title compound were obtained.

LC-MS (Method 9): Rₜ=1.01 min; MS (ESIpos): m/z=215 [M+H]⁺

### Intermediate 269

### 3,5-difluoro-2-(4-methyl-2H-1,2,3-triazol-2-yl)pyridine

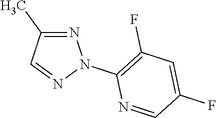

N-{(1E,2Z)-2-[2-(3,5-difluoropyridin-2-yl)hydrazinylidene]propylidene}hydroxylamine (601 mg, 38% purity, 1.05 mmol) was dissolved in 10 ml of acetic anhydride and stirred at 130° C. After 3 h Isolute® was added and the solvent was evaporated on a rotary evaporator. The crude product was purified by column chromatography (Machine: Biotage® Isolera One; column: Biotage® SNAP Ultra 50 g; gradient: Cy/EE-gradient, 8% EE-66% EE; flow: 100 ml/min). Product containing samples were united and the solvents were removed on a rotary evaporator. 249 mg (68% purity, 82% yield) of the title compound were obtained.

LC-MS (Method 7): Rₜ=1.19 min; MS (ESIpos): m/z=197 [M+H]⁺

### Intermediate 270

### 2-(3,5-difluoropyridin-2-yl)-2H-1,2,3-triazole-4-carboxylic acid

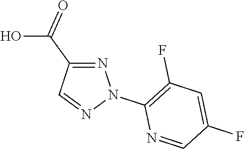 [SMILES: O=C(O)c1cnn(-c2ncc(F)cc2F)n1]

3,5-difluoro-2-(4-methyl-2H-1,2,3-triazol-2-yl)pyridine (249 mg, 68% purity, 863 μmol) was suspended in 10 ml of water. Potassium permanganate (682 mg, 4.32 mmol) was added and the mixture was stirred at 100° C. After 48 h more potassium permanganate (500 mg, 3.16 mmol) was added and the mixture was stirred at 100° C. for 24 h. Once again more potassium permanganate (500 mg, 3.16 mmol) was added and the mixture was stirred for another 5 d at 100° C. The mixture was filtered over Celite® and the filtrate was purified by preparative HPLC (Column: Chromatorex C18 10 μm 250×30 mm; eluent A=water, B=acetonitrile; gradient: 0.0 min 5% B; 3 min 5% B; 20 min 50% B; 23 min 100% B; 26 min 5% B; flow: 50 ml/min; 0.1% formic acid). Product containing samples were united and the solvents were removed on a rotary evaporator. 8.00 mg (100% purity, 4% yield) of the title compound were obtained.

LC-MS (Method 7): Rₜ=0.72 min; MS (ESIpos): m/z=227 [M+H]⁺

### Intermediate 271

### tert-butyl [2-oxo-2-(1,3-thiazol-2-yl)ethyl]carbamate

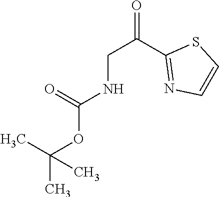 [SMILES: CC(C)(C)OC(=O)NCC(=O)c1nccs1]

Under argon, thiazole (3.6 ml, 51 mmol) was dissolved in THF (20 ml) and cooled in an acetonitrile/dry ice bath to −40° C. The solution was treated dropwise with n-butyllithium in hexane (20.3 ml, 2.5 M, 51 mmol). After 45 minutes at −35° C.; a solution of tert-butyl {2-[methoxy(methyl)amino]-2-oxoethyl}carbamate (5.00 g, 22.9 mmol) in 30 ml of THF was added dropwise, while keeping the temperature below −40° C. After 2 h at −40 to −45° C.; the reaction was quenched with 1 N citric acid (25 ml) and diluted with ethyl acetate. The layers were separated. The aqueous layer was extracted with ethyl acetate and the organic phases were combined, washed with brine, dried and concentrated under reduced pressure. The crude product was purified by column chromatography (100 g Ultra Snap Cartridge Biotage; eluent ethyl acetate/cyclohexane, elution gradient: 25%→60%) to give. 3.04 g (98% purity, 24% yield) of the title compound.

LC-MS (Method 8): Rₜ=0.78 min; MS (ESIpos): m/z=243 [M+H]⁺

¹H-NMR (500 MHz, DMSO-d6) δ [ppm]: 1.263 (0.77), 1.398 (16.00), 4.515 (1.84), 4.527 (1.80), 7.182 (0.52), 8.167 (1.36), 8.173 (1.47), 8.257 (1.73), 8.263 (1.52).

### Intermediate 272

### rac-tert-butyl {[2,5-dioxo-4-(1,3-thiazol-2-yl)imidazolidin-4-yl]methyl}carbamate

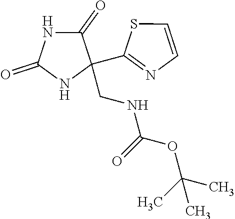 [SMILES: CC(C)(C)OC(=O)NCC1(c2nccs2)NC(=O)NC1=O]

To a solution of tert-butyl [2-oxo-2-(1,3-thiazol-2-yl)ethyl]carbamate (3.04 g, 12.5 mmol) in methanol (20 ml) was added potassium cyanide (3.27 g, 50.2 mmol) and ammonium carbonate (4.82 g, 50.2 mmol) at RT. The reaction mixture was stirred overnight at 40° C. into a sealed pressure flask and then diluted with water. The resulting suspension was extracted with dichloromethane. After phase separation; the aqueous phase was concentrated under reduced pressure and the crude product was suspended in a mixture of 30 ml acetonitrile and 20 ml methanol. After filtration of the residue, the filtrate was concentrated in vacuo and 1.4 g of the desired product was obtained and used without further purification.

LC-MS (Method 7): Rₜ=0.99 min; MS (ESIneg): m/z=311 [M−H]⁻

The title compound can also be synthesized via the procedure described in J. Med. Chem. 2014, 57, 10476-10485.

### Intermediate 273

### rac-5-(aminomethyl)-5-(1,3-thiazol-2-yl)imidazolidine-2,4-dione hydrochloride

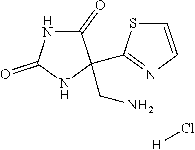 [SMILES: NCC1(c2nccs2)NC(=O)NC1=O ; Cl]

To a solution of rac-tert-butyl {[2,5-dioxo-4-(1,3-thiazol-2-yl)imidazolidin-4-yl]methyl}carbamate (1.40 g, 4.48 mmol) in methanol (28 ml) were added 22.4 ml (90 mmol) of 4M hydrochloric acid in dioxane at 0° C. After 1 h at 0° C., the reaction mixture was stirred overnight at RT and concentrated in vacuo. 1.30 g of the title compound was used without further purification.

LC-MS (Method 9): Rₜ=0.26 min; MS (ESIneg): m/z=211 [M−HCl−H]⁻

¹H-NMR (600 MHz, DMSO-d6) δ [ppm]: 0.005 (1.86), 1.234 (0.56), 1.246 (0.80), 1.596 (0.54), 1.759 (0.78), 2.387 (0.62), 2.426 (1.00), 2.655 (0.64), 2.887 (0.46), 3.167 (0.62), 3.462 (0.48), 3.472 (0.58), 3.492 (0.52), 3.502 (0.56), 3.527 (1.42), 3.537 (1.68), 3.550 (1.90), 3.559 (1.52), 3.667 (0.54), 3.676 (0.42), 3.704 (0.58), 3.713 (0.54), 3.722 (1.86), 3.754 (1.74), 3.763 (1.94), 3.776 (1.62), 3.785 (1.34), 3.917 (0.52), 5.084 (1.26), 7.183 (2.00), 7.268 (2.21), 7.352 (2.00), 7.774 (0.52), 7.780 (0.46), 7.881 (10.95), 7.886 (15.10), 7.909 (16.00), 7.914 (10.55), 8.484 (5.59), 9.120 (5.57), 11.420 (5.69).

The title compound can also be synthesized via the procedure described in J. Med. Chem. 2014, 57, 10476-10485.

### Intermediate 274

### tert-Butyl [2-(1-methyl-1H-imidazol-2-yl)-2-oxoethyl]carbamate

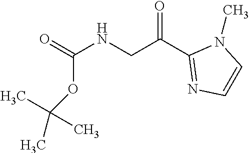 [SMILES: Cn1ccnc1C(=O)CNC(=O)OC(C)(C)C]

A solution of 2.70 mL (6.70 mmol) of 2.5M n-butyllithium in hexanes was added to a solution of 0.49 mL (6.10 mmol) of 1-methyl-1H-imidazole in 10 mL of anhydrous tetrahydrofuran under nitrogen atmosphere at 0° C. over 5 min and the resulting yellow solution was stirred under these conditions for 10 min. After that, the lithiated imidazole solution was transferred to a solution of 1.46 g (6.70 mmol) of tert-butyl {2-[methoxy(methyl)amino]-2-oxoethyl}carbamate at −78° C. over 15 min and the corresponding solution was further stirred for 1 hour under the same conditions. After that time, the solution was taken out of the cooling bath and it was stirred for 15 min while warming up to room temperature. The mixture was quenched with 5 mL of a 1M solution of hydrochloric acid(aq.), stirred for 5 min, and 5 mL of brine and 5 mL of saturated hydrogencarbonate(aq.) were added. The resulting mixture was transferred to a separatory funnel and extracted with ethyl acetate. The organic layer was dried over magnesium sulfate, filtered and evaporated to dryness to give 0.70 g (48%) of the product as a pale orange oil. The compound was pure enough to be used as such without further purification.

¹H-NMR (300 MHz, DMSO-d₆): δ [ppm]=1.39 (s, 9H), 3.91 (s, 3H), 4.40 (d, 2H), 6.99 (t, 1H), 7.11 (s, 1H), 7.52 (s, 1H).

LC-MS (Method 3): Rₜ=0.546 min. MS (Mass method 1): m/z=240 (M+H)⁺

### Intermediate 275

### rac-tert-Butyl {[4-(1-methyl-1H-imidazol-2-yl)-2,5-dioxoimidazolidin-4-yl]methyl}carbamate

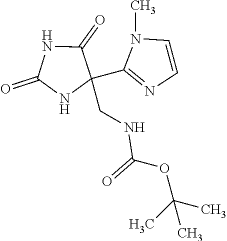 [SMILES: Cn1ccnc1C1(CNC(=O)OC(C)(C)C)NC(=O)NC1=O]

To a stirring solution of 4.22 g (43.90 mmol) of ammonium carbonate and 0.63 g (11.70 mmol) of ammonium chloride in 10 mL of water was added 0.70 g (2.93 mmol) of tert-butyl [2-(1-methyl-1H-imidazol-2-yl)-2-oxoethyl]carbamate in 10 mL of ethanol. After 15 min, 0.86 g (13.20 mmol) of potassium cyanide were added and the mixture was heated up to 60° C. for 16 hours into a sealed pressure flask. The yellow solution was concentrated until only a small fraction of water remained (a white precipitate appeared). Then, more water was added and the suspension was allowed to stand at 0° C. for 2 hour. After that time, the resulting solid was filtered off and washed with cold water and diethyl ether to give 0.90 g (98%) of the product as an off-white powder. The compound was used as such in the next step.

LC-MS (Method 3): Rₜ=0.253 min. MS (Mass method 1): m/z=310 (M+H)⁺

### Intermediate 276

### rac-5-(aminomethyl)-5-(1-methyl-1H-imidazol-2-yl)imidazolidine-2,4-dione hydrochloride

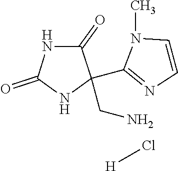

3.60 mL (15.00 mmol) of 4N hydrochloric acid in dioxane were added to a solution of 0.90 g (2.91 mmol) of rac-tert-Butyl {[4-(1-methyl-1H-imidazol-2-yl)-2,5-dioxoimidazolidin-4-yl]methyl}carbamate in 20 mL of dichloromethane and the mixture was allowed to stir at room temperature for 16 hours. The resulting precipitate was collected by filtration and washed with dichloromethane, ethyl acetate and diethyl ether to give 0.71 g (99%) of the product as a white solid. The compound was used as such in the next step.

LC-MS (Method 3): Rₜ=0.089 min. MS (Mass method 1): m/z=157 (M+H)⁺

### Intermediate 277

### ethyl 5-(1,3-thiazol-4-yl)-1,3-oxazole-4-carboxylate

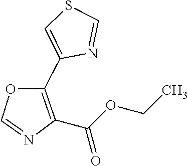

1,3-thiazole-4-carboxylic acid (1.00 g, 7.74 mmol) dissolved in 12 ml of THF was treated with 1,1,-carbonyl-diimidazole (1.51 g, 9.29 mmol) and stirred at room temperature. After 2 h, ethyl isocyanoacetate (930 μl, 8.5 mmol), dissolved in 12 ml of THF, and a solution of lithium bis(trimethylsilyl)amide in THF (7.7 ml, 1.0 M, 7.7 mmol) were added at 0° C. The mixture was allowed to warm to room temperature, stirred overnight and then concentrated in vacuo. The residue was taken up with ethyl acetate and washed with water. The organic layer was dried and concentrated in vacuo. The crude product was purified by column chromatography (100 g Ultra Snap Cartridge Biotage; eluent ethyl acetate/cyclohexane, elution gradient 15%→100%).

Samples containing the desired product were united, the solvents were evaporated and the residue was dried in vacuum. 783 mg (44% yield) of the title compound was used without further purification.

LC-MS (Method 8): Rₜ=0.65 min; MS (ESIpos): m/z=225 [M+H]⁺

¹H-NMR (600 MHz, DMSO-d6) δ [ppm]: 1.291 (7.74), 1.302 (16.00), 1.314 (7.70), 4.313 (2.51), 4.325 (7.74), 4.337 (7.62), 4.349 (2.39), 8.580 (9.32), 8.744 (5.07), 8.747 (5.10), 9.273 (4.82), 9.276 (4.76).

### Intermediate 278

### 2-amino-1-(1,3-thiazol-4-yl)ethanone hydrochloride

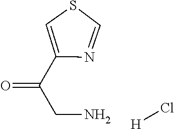

ethyl 5-(1,3-thiazol-4-yl)-1,3-oxazole-4-carboxylate (782 mg, 3.49 mmol) was taken up in 19 ml of 6 N hydrochloric acid. After 2 h the resulting mixture was concentrated in vacuo and the residue was treated with DCM and a small amount of methanol. The precipitate was filtered off and dried in vacuo. 613 mg (98% yield) of the title compound was obtained and used without further purification

LC-MS (Method 9): Rₜ=0.48 min; MS (ESIpos): m/z=143 [M−HCl+H]⁺

¹H-NMR (600 MHz, DMSO-d₆) δ[ppm]: 1.067 (4.56), 1.079 (9.66), 1.091 (4.71), 2.525 (0.47), 4.145 (1.10), 4.156 (3.13), 4.168 (3.10), 4.180 (1.18), 4.437 (4.70), 4.479 (5.69), 4.488 (11.43), 4.497 (11.15), 5.938 (1.13), 8.478 (5.26), 8.827 (15.55), 8.830 (16.00), 8.982 (3.87), 8.985 (4.11), 9.202 (1.56), 9.303 (15.03), 9.307 (15.02), 9.322 (4.08), 9.325 (4.09).

### Intermediate 279

### tert-butyl [2-oxo-2-(1,3-thiazol-4-yl)ethyl]carbamate

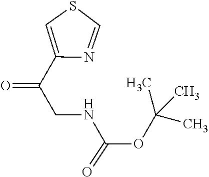 [SMILES: CC(C)(C)OC(=O)NCC(=O)c1cscn1]

To a solution of 2-amino-1-(1,3-thiazol-4-yl)ethanone hydrochloride (610 mg, 3.41 mmol) in dichloromethane (14 ml) was added di-tert-butyl dicarbonate (860 μl, 3.8 mmol) and triethylamine (1.4 ml, 10 mmol). After 1.5 h of stirring at RT, the reaction mixture was concentrated under reduced pressure, diluted with ethylacetate and washed with water and brine. After phase separation, the organic phase was dried, concentrated in vacuo and 781 mg of the title compound was used without further purification.

LC-MS (Method 7): Rₜ=1.33 min; MS (ESIpos): m/z=143 [M−Boc+H]

¹H-NMR (400 MHz, DMSO-d6) δ [ppm]: −0.008 (0.42), 0.008 (0.42), 1.093 (0.66), 1.110 (0.45), 1.282 (0.71), 1.306 (0.40), 1.366 (0.48), 1.387 (2.15), 1.398 (14.02), 1.468 (16.00), 4.430 (1.68), 4.445 (1.65), 7.061 (0.46), 8.635 (0.72), 8.640 (0.78), 9.236 (0.92), 9.240 (0.90).

### Intermediate 280

### rac-tert-butyl {[2,5-dioxo-4-(1,3-thiazol-4-yl)imidazolidin-4-yl]methyl}carbamate

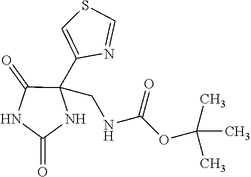 [SMILES: CC(C)(C)OC(=O)NCC1(c2cscn2)NC(=O)NC1=O]

To a solution of tert-butyl [2-oxo-2-(1,3-thiazol-4-yl)ethyl]carbamate (781 mg, 3.22 mmol) in methanol (5.9 ml) was added potassium cyanide (840 mg, 12.9 mmol) and ammonium carbonate (1.24 g, 12.9 mmol) at RT. The reaction mixture was stirred for 2 days at 40° C. into a sealed pressure flask. After filtration at RT, the resulting filtrate was concentrated in vacuo. The residue was purified by preparative HPLC (Method 2f). 283 mg of the desired product was obtained and used without further purification

LC-MS (Method 8): Rₜ=0.63 min; MS (ESIneg): m/z=311 [M−H]⁻

¹H-NMR (400 MHz, DMSO-d6) δ [ppm]: 1.366 (16.00), 1.434 (0.81), 7.733 (1.28), 7.738 (1.32), 8.136 (0.77), 9.107 (0.96), 9.111 (0.97), 10.776 (0.53).

### Intermediate 281

### rac-5-(aminomethyl)-5-(1,3-thiazol-4-yl)imidazolidine-2,4-dione hydrochloride

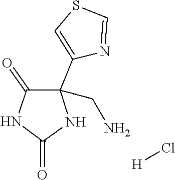

To a solution of rac-tert-butyl {[2,5-dioxo-4-(1,3-thiazol-4-yl)imidazolidin-4-yl]methyl}carbamate (283 mg, 906 μmol) in dichloromethane (7.8 ml) were added 1.1 ml of 4M hydrochloric acid in dioxane at RT. The reaction mixture was stirred for 4 h and concentrated in vacuo. 200 mg (84% yield) of the title compound was used without further purification.

LC-MS (Method 9): Rₜ=0.24 min; MS (ESIpos): m/z=213 [M−HCl+H]⁺

¹H-NMR (400 MHz, DMSO-d6) δ [ppm]: −0.008 (2.08), 0.008 (2.39), 1.366 (1.42), 1.596 (1.40), 2.525 (0.83), 3.433 (0.84), 3.446 (1.03), 3.466 (1.26), 3.479 (1.11), 3.568 (16.00), 3.637 (0.42), 3.660 (1.41), 3.676 (1.25), 3.695 (1.03), 3.709 (0.84), 3.872 (0.44), 4.304 (1.61), 5.755 (1.98), 7.647 (0.43), 7.652 (0.46), 7.895 (8.71), 7.900 (8.69), 8.343 (3.74), 8.524 (4.89), 9.180 (4.89), 9.185 (4.86), 11.201 (4.04).

### Intermediate 282

### Ethyl (2E)-2-[2-(2-chloro-4-fluorophenyl)hydrazinylidene]-3-oxopropanoate

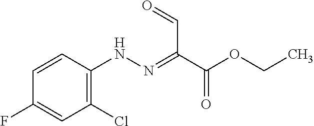 [SMILES: CCOC(=O)/C(C=O)=N/Nc1ccc(F)cc1Cl]

2-chloro-4-fluoroaniline (210 μl, 1.7 mmol) was dissolved in 2.5 ml of water and 470 μl of concentrated hydrochloric acid. The mixture was cooled to 0° C. and a solution of sodium nitrite (118 mg, 1.72 mmol) in 1.25 ml of water was added dropwise. After stirring for 5 minutes this solution was added dropwise to a solution of ethyl-(2E)-3-(dimethylamino)acrylate (270 μl, 1.9 mmol) and potassium acetate (253 mg, 2.58 mmol) in 3.75 ml of ethanol at 0° C. The mixture was stirred at room temperature for 30 min before being diluted with ethyl acetate and washed twice with water and once with brine. The organic layer was dried over sodium sulfate, filtered and evaporated. 427 mg (46% purity, 42% yield) of the title compound were obtained.

LC-MS (Method 7): Rₜ=2.09 min; MS (ESIneg): m/z=271 [M−H]⁻

### Intermediate 283

### Ethyl (2E,3E)-2-[2-(2-chloro-4-fluorophenyl)hydrazinylidene]-3-(hydroxyimino)propanoate

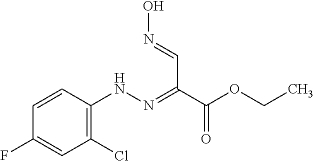 [SMILES: CCOC(=O)C(/C=N/O)=N/Nc1ccc(F)cc1Cl]

Ethyl (2E)-2-[2-(2-chloro-4-fluorophenyl)hydrazinylidene]-3-oxopropanoate (417 mg, 1.53 mmol), hydroxylamine hydrochloride (128 mg, 1.84 mmol) and potassium acetate (375 mg, 3.82 mmol) were dissolved in 8.7 ml of ethanol and the mixture was stirred at room temperature over night. The reaction mixture was diluted with ethyl acetate and washed twice with water and once with brine. The organic layer was dried over sodium sulfate, filtered and evaporated. The residue was dried in vacuum. 341 mg (75% purity, 58% yield) of the title compound were obtained.

LC-MS (Method 7): Rₜ=1.92 min; MS (ESIpos): m/z=288 [M+H]⁺

### Intermediate 284

### Ethyl 2-(2-chloro-4-fluorophenyl)-2H-1,2,3-triazole-4-carboxylate

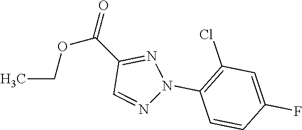 [SMILES: CCOC(=O)c1cnn(-c2ccc(F)cc2Cl)n1]

Ethyl (2E,3E)-2-[2-(2-chloro-4-fluorophenyl)hydrazinylidene]-3-(hydroxyimino)propanoate (330 mg, 1.15 mmol) was stirred for 2 h in 4 ml of acetic acid anhydride at 60° C. The reaction was then diluted in ethyl acetate and washed twice with water and once with brine. The organic layer was dried over sodium sulfate, filtered and evaporated. The residue was adsorbed on Isolute® and purified by chromatography on silica gel (10 g Ultra Snap Cartridge Biotage®; Biotage-Isolera-One®; Cy/EE-gradient: 2% EE-20% EE; flow: 36 ml/min). Samples containing the desired product were united, the solvents were evaporated and the residue was dried in vacuum. 37.8 mg (98% purity, 12% yield) of the title compound were obtained.

LC-MS (Method 7): Rₜ=1.92 min; MS (ESIpos): m/z=270 [M+H]⁺

### Intermediate 285

### 2-(2-chloro-4-fluorophenyl)-2H-1,2,3-triazole-4-carboxylic acid

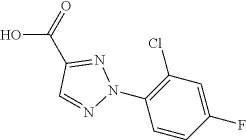 [SMILES: O=C(O)c1cnn(-c2ccc(F)cc2Cl)n1]

Ethyl 2-(2-chloro-4-fluorophenyl)-2H-1,2,3-triazole-4-carboxylate (37.8 mg, 140 μmol) was dissolved in 8 ml of THF, treated with lithium hydroxide solution (140 μl, 1.0 M, 140 μmol) and the mixture was stirred for 1 h at room temperature. More lithium hydroxide solution (140 μl, 1.0 M, 140 μmol) was added and the mixture was stirred over night at room temperature. The solvent was removed on a rotary evaporator. Water was added and the mixture was acidified with 2 N hydrochloric acid. The resulting precipitate was collected by filtration. 19.9 mg (96% purity, 56% yield) of the title compound were obtained.

LC-MS (Method 7): Rₜ=1.24 min; MS (ESIneg): m/z=240 [M−H]⁻

### Intermediate 286

### rac-tert-butyl [(4-methyl-2,5-dioxoimidazolidin-4-yl)methyl]carbamate

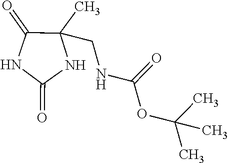 [SMILES: CC(C)(C)OC(=O)NCC1(C)NC(=O)NC1=O]

The reaction was performed in 2 Microwave Vials. To a solution of 1.85 g (34.6 mmol) of ammonium chloride and 12.5 g (130 mmol) of ammonium carbonate in 7.5 mL of water was added a solution of 1.50 g (8.66 mmol) of tert-butyl (2-oxopropyl)carbamate in 7.5 ml ethanol.

The reaction mixture was stirred 15 minutes at room temperature and then 2.54 g (39.0 mmol) of potassium cyanide were added into the mixture and the vials were sealed. The mixture was stirred at 65° C. for 24 hours. After that time, the solvent was partially removed and the mixture was solved in water and extracted with ethyl acetate. The resulting organic phase was washed with Brine, filtered over an hydrophobic filter and evaporated under vacuo to give 1.69 g (79%) of the product which was used as such in the next step.

¹H-NMR (400 MHz, DMSO-d6) δ [ppm]: −0.008 (0.42), 0.008 (0.40), 1.186 (4.35), 1.371 (16.00), 3.129 (0.51), 3.145 (0.48), 7.626 (0.69).

LC-MS (Method 7): Rₜ=0.85 min; MS (ESIneg): m/z=242 [M−H]⁻

### Intermediate 287

### rac-5-(aminomethyl)-5-methylimidazolidine-2,4-dione-hydrogen chloride

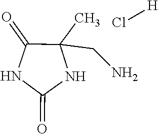

1.70 g (6.95 mmol) of rac-tert-butyl [(4-methyl-2,5-dioxoimidazolidin-4-yl)methyl]carbamate was dissolved in 25 mL of dioxane and 26.05 mL (104.21 mmol) of 4M hydrochloric acid in dioxane were added and the resulting suspension was stirred at room temperature for 16 hours. After that time, the solvent was evaporated and dried under vacuo to give 1.18 g (100%) of the product. The compound was used as such in the next step.

¹H-NMR (400 MHz, DMSO-d₆) δ[ppm]: 1.343 (16.00), 2.908 (0.74), 2.942 (1.07), 3.074 (1.08), 3.107 (0.75), 8.012 (1.56), 8.215 (1.60), 10.948 (1.58).

LC-MS (Method 9): Rₜ=0.25 min; MS (ESIpos): m/z=144 [M+H-HCl]⁺

### Intermediate 288

### tert-butyl {2-oxo-2-[3-(trifluoromethyl)pyridin-2-yl]ethyl}carbamate

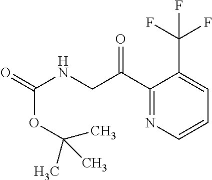 [SMILES: CC(C)(C)OC(=O)NCC(=O)c1ncccc1C(F)(F)F]

To a solution of 2-amino-1-[3-(trifluoromethyl)pyridin-2-yl]ethanone hydrochloride (2.00 g, 8.31 mmol) in dichloromethane (37 ml) was added di-tert-butyl dicarbonate (2.00 g, 9.14 mmol) and triethylamine (3.5 ml, 25 mmol). After 2 h of stirring at RT, the reaction mixture was diluted with dichloromethane and extracted with water. After phase separation, the organic phase was dried; concentrated in vacuo and 2.30 g (91% yield) of the title compound was obtained.

LC-MS (Method 9): Rₜ=1.58 min; MS (ESIpos): m/z=205 [M−Boc+H]⁺

¹H-NMR (400 MHz, DMSO-d6) δ [ppm]: 1.284 (0.44), 1.322 (1.44), 1.371 (0.52), 1.394 (16.00), 4.481 (1.92), 4.496 (1.89), 7.197 (0.61), 7.842 (0.57), 7.853 (0.62), 7.862 (0.65), 7.874 (0.65), 8.378 (0.80), 8.399 (0.76), 8.939 (0.88), 8.950 (0.89).

### Intermediate 289

### rac-tert-butyl ({2,5-dioxo-4-[3-(trifluoromethyl)pyridin-2-yl]imidazolidin-4-yl}methyl)carbamate

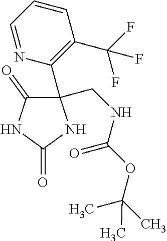 [SMILES: CC(C)(C)OC(=O)NCC1(c2ncccc2C(F)(F)F)NC(=O)NC1=O]

To a solution of tert-butyl {2-oxo-2-[3-(trifluoromethyl)pyridin-2-yl]ethyl}carbamate (2.30 g, 7.56 mmol) in methanol (13.6 ml) was added potassium cyanide (1.97 g, 30.2 mmol) and ammonium carbonate (2.91 g, 30.2 mmol) at RT. The reaction mixture was first stirred overnight at 60° C. into a sealed pressure flask. After additional 24 h stirring at 80° C., extra portion of potassium cyanide (738 mg, 11.3 mmol) and ammonium carbonate (1.09 g, 11.3 mmol) were added due to incomplete conversion. The reaction mixture was additionally stirred overnight at 80° C. After filtration at RT, the resulting filtrate was concentrated in vacuo. The residue was purified by preparative HPLC (sample preparation: 7.7 g dissolved in a mixture of methanol, water, acetonitrile; column: XBridge C18 5 μm, 100×30 mm; eluent:water→acetonitrile/water 80:20+1% ammonia solution→acetonitrile; flow rate: 80 ml/min; temperature: 40° C.; UV detection: 210 nm). 730 mg of the desired product was obtained and used without further purification.

LC-MS (Method 9): Rₜ=0.87 min; MS (ESIneg): m/z=373 [M−H]⁻

¹H-NMR (600 MHz, DMSO-d6) δ [ppm]: 1.368 (16.00), 1.378 (2.03), 1.755 (0.91), 7.624 (0.46), 7.632 (0.55), 7.644 (0.48), 8.240 (0.67), 8.253 (0.66), 8.855 (0.75), 8.862 (0.75).

### Intermediate 290

### rac-5-(aminomethyl)-5-[3-(trifluoromethyl)pyridin-2-yl]imidazolidine-2,4-dione hydrochloride

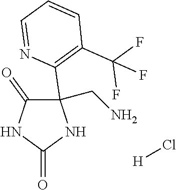 [SMILES: NCC1(c2ncccc2C(F)(F)F)NC(=O)NC1=O ; Cl]

To a solution of rac-tert-butyl ({2,5-dioxo-4-[3-(trifluoromethyl)pyridin-2-yl]imidazolidin-4-yl}methyl)carbamate (725 mg, 1.94 mmol) in dichloromethane (7.6 ml) were added 2.42 ml of 4M hydrochloric acid (9.7 mmol) in dioxane at 0° C. The reaction mixture was stirred for 1 h at 0° C. then for 2 h at room temperature. The precipitate was filtered off, washed with dichloromethane and dried in vacuo. 610 mg of the title compound were obtained and used without further purification.

LC-MS (Method 9): Rₜ=0.28 min; MS (ESIneg): m/z=273 [M−HCl−H]⁻

¹H-NMR (600 MHz, DMSO-d₆) δ[ppm]: 1.596 (0.55), 1.757 (2.53), 2.501 (16.00), 3.168 (2.76), 3.483 (0.75), 3.493 (0.85), 3.795 (0.96), 3.807 (0.84), 7.745 (1.54), 7.754 (1.68), 7.759 (1.72), 7.767 (1.66), 8.362 (5.66), 8.375 (3.57), 8.708 (3.61), 8.898 (2.56), 8.905 (2.54), 11.399 (3.00).

### Intermediate 291

### ethyl 5-(3,3-difluorocyclobutyl)-1,3-oxazole-4-carboxylate

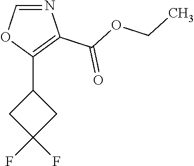 [SMILES: CCOC(=O)c1ncoc1C1CC(F)(F)C1]

3,3-difluorocyclobutane-1-carboxylic acid (3.00 g, 22.0 mmol) dissolved in 30 ml of THF was treated with 1,1,-carbonyl-diimidazole (4.29 g, 26.5 mmol) and stirred at room temperature. After 1 h, ethyl isocyanoacetate (2.7 ml, 24 mmol), dissolved in 30 ml of THF, and a solution of lithium bis(trimethylsilyl)amide in THF (22 ml, 1.0 M, 22 mmol) were added at 0° C. The mixture was allowed to warm to room temperature, stirred overnight and then concentrated in vacuo. The residue was taken up with ethyl acetate and washed with water. The organic layer was dried and concentrated in vacuo. The crude product was purified by column chromatography (340 g Ultra Snap Cartridge Biotage; eluent ethyl acetate/cyclohexane, elution gradient 8%→58%). Samples containing the desired product were united, the solvents were evaporated and the residue was dried in vacuum. 2.50 g (100% purity, 49% yield) of the title compound was used without further purification.

LC-MS (Method 9): Rₜ=1.39 min; MS (ESIpos): m/z=232 [M+H]⁺

¹H-NMR (400 MHz, DMSO-d6) δ [ppm]: 1.272 (7.71), 1.290 (16.00), 1.308 (7.89), 2.831 (0.57), 2.852 (0.60), 2.864 (0.99), 2.868 (1.15), 2.873 (0.96), 2.884 (1.04), 2.889 (1.24), 2.893 (1.23), 2.901 (1.49), 2.906 (1.08), 2.910 (1.36), 2.921 (1.29), 2.926 (1.03), 2.930 (1.27), 2.943 (0.91), 2.963 (0.87), 3.004 (0.83), 3.013 (0.41), 3.022 (0.97), 3.027 (1.24), 3.031 (0.94), 3.038 (1.36), 3.045 (1.30), 3.061 (1.74), 3.075 (1.07), 3.080 (1.33), 3.088 (0.76), 3.093 (0.89), 3.098 (0.67), 3.116 (0.57), 4.005 (0.79), 4.026 (1.16), 4.032 (1.16), 4.047 (0.74), 4.249 (2.55), 4.267 (7.82), 4.285 (7.74), 4.303 (2.49), 8.450 (6.13).

### Intermediate 292

### 2-amino-1-(3,3-difluorocyclobutyl)ethanone hydrochloride

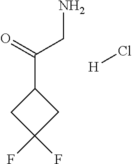

Ethyl 5-(3,3-difluorocyclobutyl)-1,3-oxazole-4-carboxylate (2.50 g, 10.8 mmol) was taken up in 70 ml of 6 N hydrochloric acid and stirred for 2 h at 100° C., then overnight at room temperature. The resulting mixture was concentrated in vacuo. Due to incomplete conversion, the crude was taken up in 70 ml of 6 N hydrochloric acid, stirred for 2 h at 100° C. and concentrated in vacuo. 1.77 g of the title compound was obtained and used without further purification.

LC-MS (Method 9): Rₜ=0.82 min; MS (ESIpos): m/z=150 [M−HCl+H]⁺

¹H-NMR (400 MHz, DMSO-d6) δ [ppm]: −0.008 (1.34), 0.008 (1.24), 1.267 (0.48), 2.329 (0.48), 2.368 (0.73), 2.524 (2.03), 2.671 (0.48), 2.712 (1.34), 2.728 (0.98), 2.744 (4.36), 2.750 (2.01), 2.763 (5.03), 2.768 (5.47), 2.771 (5.05), 2.774 (5.44), 2.782 (7.33), 2.791 (9.26), 2.803 (9.07), 2.812 (7.08), 2.819 (5.74), 2.826 (8.00), 2.834 (4.36), 2.841 (3.64), 2.847 (3.75), 2.858 (1.51), 2.881 (0.78), 3.167 (1.28), 3.295 (0.80), 3.301 (0.82), 3.316 (2.32), 3.323 (2.55), 3.338 (3.25), 3.343 (3.54), 3.361 (2.01), 3.366 (2.03), 3.380 (0.63), 3.386 (0.54), 3.961 (6.24), 3.975 (16.00), 3.989 (15.43), 4.003 (4.82), 8.212 (5.51).

### Intermediate 293

### tert-butyl [2-(3,3-difluorocyclobutyl)-2-oxoethyl]carbamate

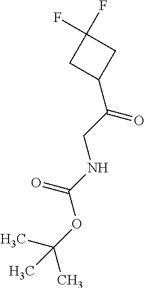 [SMILES: CC(C)(C)OC(=O)NCC(=O)C1CC(F)(F)C1]

To a solution of 2-amino-1-(3,3-difluorocyclobutyl)ethanone hydrochloride (1.77 g, 9.54 mmol) in dichloromethane (43 ml) was added di-tert-butyl dicarbonate (2.29 g, 10.5 mmol) and triethylamine (4.0 ml, 29 mmol). After 2 h of stirring at RT, the reaction mixture was diluted with dichloromethane and extracted with water. After phase separation, the organic phase was dried;

concentrated in vacuo and 2.50 g (88% purity) of the title compound was obtained and used without further purification.

¹H-NMR (400 MHz, DMSO-d6) δ [ppm]: 1.382 (5.06), 1.468 (16.00), 2.517 (0.48), 3.324 (0.46), 3.789 (0.73), 3.804 (0.73).

### Intermediate 294

### rac-tert-butyl {[4-(3,3-difluorocyclobutyl)-2,5-dioxoimidazolidin-4-yl]methyl}carbamate

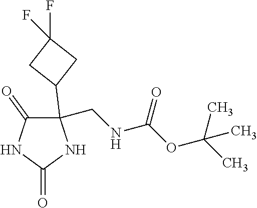 [SMILES: CC(C)(C)OC(=O)NCC1(C2CC(F)(F)C2)NC(=O)NC1=O]

To a solution of tert-butyl [2-(3,3-difluorocyclobutyl)-2-oxoethyl]carbamate (2.50 g, 88% purity, 8.83 mmol) in methanol (16 ml) was added potassium cyanide (2.30 g, 35.3 mmol) and ammonium carbonate (3.39 g, 35.3 mmol) at RT. The reaction mixture was first stirred overnight at 80° C. into a sealed pressure flask. Extra portion of potassium cyanide (1.15 g, 17.7 mmol) aid ammonium carbonate (1.70 g, 17.7 mmol) were added due to incomplete conversion. The reaction mixture was additionally stirred overnight at 80° C.

After filtration and washing of the suspension with methanol at RT, the resulting filtrate was concentrated in vacuo. The residue was purified by preparative HPLC (sample preparation: 6.4 g dissolved in a mixture of methanol, water, acetonitrile; column: XBridge C18 5 μm, 100×30 mm; eluent:water→acetonitrile/water 80:20+1% ammonia solution→acetonitrile; flow rate: 80 ml/min; temperature: 40° C.; UV detection: 210 nm). 1.68 g of the desired product was obtained and used without further purification.

LC-MS (Method 9): Rₜ=0.92 min; MS (ESIneg): m/z=318 [M−H]⁻

### Intermediate 295

### rac-5-(aminomethyl)-5-(3,3-difluorocyclobutyl)imidazolidine-2,4-dione hydrochloride

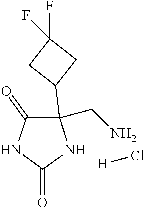

To a solution of rac-tert-butyl {[4-(3,3-difluorocyclobutyl)-2,5-dioxoimidazolidin-4-yl]methyl}carbamate (1.68 g, 5.26 mmol) in dichloro methane (21 ml) were added 6.6 ml of 4M hydrochloric acid (26 mmol) in dioxane at 0° C. The reaction mixture was stirred for 1 h at 0° C. then for 2 h at room temperature. The precipitate was filtered off, washed with dichloromethane and dried in vacuo. 1.46 g of the title compound was obtained and used without further purification.

LC-MS (Method 9): Rₜ=0.29 min; MS (ESIpos): m/z=220 [M−HCl+H]⁺

¹H-NMR (600 MHz, DMSO-d6) δ [ppm]: 2.316 (0.52), 2.333 (0.53), 2.347 (0.45), 2.518 (1.08), 2.575 (0.84), 2.587 (1.27), 2.597 (1.19), 2.619 (0.73), 2.641 (0.62), 2.659 (0.75), 2.666 (0.72), 2.674 (0.61), 2.682 (0.51), 2.689 (0.41), 2.910 (1.65), 2.932 (2.01), 3.114 (2.03), 3.137 (1.66), 3.568 (16.00), 8.324 (2.83), 8.366 (3.74), 11.128 (1.25).

### Intermediate 296

### ethyl 5-(1-fluorocyclopropyl)-1,3-oxazole-4-carboxylate

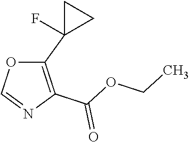

1-fluorocyclopropane-1-carboxylic acid (3.00 g, 28.8 mmol) dissolved in 45 ml of THF was treated with 1,1,-carbonyl-diimidazole (5.61 g, 34.6 mmol) and stirred at room temperature. After 2 h, ethyl isocyanoacetate (3.5 ml, 32 mmol), dissolved in 45 ml of THF, and a solution of lithium bis(trimethylsilyl)amide in THF (29 ml, 1.0 M, 29 mmol) were added at 0° C. The mixture was allowed to warm to room temperature, stirred overnight and then concentrated in vacuo. The residue was taken up with ethyl acetate and washed with water. The organic layer was dried and concentrated in vacuo. The crude product was purified by column chromatography (340 g Ultra Snap Cartridge Biotage; eluent ethyl acetate/cyclohexane, elution gradient 10%→65%). Samples containing the desired product were united, the solvents were evaporated and the residue was dried in vacuum. 2.84 g (100% purity, 49% yield) of the title compound was used without further purification.

LC-MS (Method 7): Rₜ=1.37 min; MS (ESIpos): m/z=200 [M+H]⁺

¹H-NMR (500 MHz, DMSO-d6) δ [ppm]: 1.205 (0.54), 1.226 (7.72), 1.240 (16.00), 1.254 (7.77), 1.264 (0.41), 1.308 (0.68), 1.421 (0.81), 1.428 (0.83), 1.439 (1.20), 1.445 (1.15), 1.452 (0.62), 1.456 (1.04), 1.461 (0.66), 1.467 (1.15), 1.475 (1.00), 1.480 (0.83), 1.483 (0.77), 1.490 (0.81), 1.493 (0.80), 1.528 (0.66), 1.531 (0.78), 1.538 (0.85), 1.543 (1.22), 1.548 (0.95), 1.554 (1.08), 1.559 (0.97), 1.563 (0.70), 1.570 (0.53), 1.577 (0.58), 1.579 (0.54), 1.585 (0.51), 1.596 (0.75), 1.603 (0.81), 1.621 (0.40), 1.632 (0.73), 1.639 (0.74), 1.652 (0.71), 1.676 (2.04), 1.679 (2.23), 1.689 (2.12), 1.712 (2.11), 1.715 (2.23), 1.722 (1.82), 1.725 (1.63), 4.220 (1.69), 4.234 (0.92), 4.242 (1.04), 4.249 (0.96), 4.256 (3.51), 4.263 (0.62), 4.269 (5.01), 4.276 (0.59), 4.283 (3.52), 4.290 (0.91), 4.297 (0.97), 4.305 (0.81), 4.307 (0.45), 5.551 (4.54), 5.554 (4.40), 9.124 (1.25).

### Intermediate 297

### 2-amino-1-(1-fluorocyclopropyl)ethanone hydrochloride

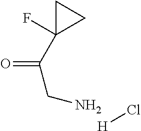

ethyl 5-(1-fluorocyclopropyl)-1,3-oxazole-4-carboxylate (2.84 g, 14.3 mmol) was taken up in 70 ml of 6 N hydrochloric acid and stirred for 2 h at 100° C. The resulting mixture was concentrated in vacuo. 1.88 g (100% purity, 86% yield) of the title compound was obtained and used without further purification.

¹H-NMR (400 MHz, DMSO-d6) δ [ppm]: 1.241 (0.49), 1.402 (2.22), 1.418 (6.53), 1.426 (7.45), 1.439 (9.53), 1.447 (7.17), 1.461 (3.46), 1.485 (0.57), 1.506 (0.48), 1.537 (0.47), 1.582 (3.31), 1.596 (6.52), 1.604 (6.02), 1.620 (2.26), 1.627 (3.14), 1.641 (6.56), 1.650 (6.39), 1.665 (2.14), 4.235 (16.00), 8.356 (5.64).

### Intermediate 298

### tert-butyl [2-(1-fluorocyclopropyl)-2-oxoethyl]carbamate

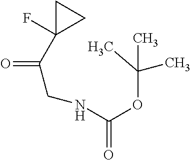

To a solution of 2-amino-1-(1-fluorocyclopropyl)ethanone hydrochloride (1.88 g, 12.2 mmol) in dichloromethane (55 ml) was added di-tert-butyl dicarbonate (2.94 g, 13.5 mmol) and triethylamine (5.1 ml, 37 mmol). After 1.5 h of stirring at RT, the reaction mixture was washed with water. After phase separation, the organic phase was dried; concentrated in vacuo and 2.48 g of the title compound was obtained and used without further purification.

LC-MS (Method 9): Rₜ=1.39 min; MS (ESIpos): m/z=218 [M+H]⁺

### Intermediate 299

### rac-tert-butyl {[4-(1-fluorocyclopropyl)-2,5-dioxoimidazolidin-4-yl]methyl}carbamate

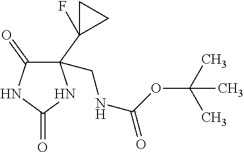 [SMILES: CC(C)(C)OC(=O)NCC1(C2(F)CC2)NC(=O)NC1=O]

To a solution of tert-butyl [2-(1-fluorocyclopropyl)-2-oxoethyl]carbamate (2.46 g, 11.3 mmol) in methanol (20 ml) was added potassium cyanide (2.95 g, 45.3 mmol) and ammonium carbonate (4.35 g, 45.3 mmol) at RT. The reaction mixture was stirred overnight at 80° C. into a sealed pressure flask. After filtration at RT, the resulting filtrate was concentrated in vacuo. The residue was purified by preparative HPLC (sample preparation: 7.4 g dissolved in a mixture of methanol, water, acetonitrile; column: XBridge C18 5 μm, 100×30 mm; eluent:water→acetonitrile/water 80:20+1% ammonia solution→acetonitrile; flow rate: 80 ml/min; temperature: 40° C.; UV detection: 220 nm). 2.31 g of the desired product was obtained and used without further purification

LC-MS (Method 9): Rₜ=0.65 min; MS (ESIneg): m/z=286 [M−H]⁻

### Intermediate 300

### rac-5-(aminomethyl)-5-(1-fluorocyclopropyl)imidazolidine-2,4-dione hydrochloride

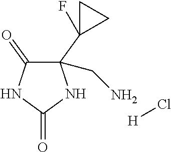

To a solution of rac-tert-butyl {[4-(1-fluorocyclopropyl)-2,5-dioxoimidazolidin-4-yl]methyl}carbamate (1.20 g, 4.18 mmol) in dichloromethane (36 ml) were added 5.2 ml (21 mmol) of 4M hydrochloric acid in dioxane at RT. The reaction mixture was stirred for 3 days and then concentrated in vacuo. 1.04 g of the title compound was used without further purification.

LC-MS (Method 9): Rₜ=0.25 min; MS (ESIpos): m/z=188 [M−HCl+H]⁺

¹H-NMR (600 MHz, DMSO-d6) δ [ppm]: 0.814 (0.66), 0.833 (2.66), 0.842 (2.91), 0.847 (3.07), 0.851 (3.10), 0.859 (2.45), 0.869 (2.25), 0.892 (0.51), 0.965 (0.55), 0.999 (0.64), 1.020 (2.80), 1.039 (5.31), 1.043 (5.25), 1.048 (3.48), 1.060 (9.17), 1.076 (2.31), 1.081 (3.58), 1.091 (5.77), 1.109 (1.82), 1.120 (0.63), 1.182 (1.41), 1.194 (2.97), 1.206 (1.66), 1.368 (11.79), 1.757 (5.46), 2.117 (2.39), 3.109 (7.54), 3.132 (9.02), 3.347 (8.25), 3.568 (4.33), 3.864 (1.61), 3.874 (1.62), 4.084 (0.44), 4.096 (1.31), 4.108 (1.33), 4.120 (0.50), 7.902 (0.50), 8.098 (0.91), 8.444 (16.00), 8.474 (9.62), 11.159 (4.19).

### Intermediate 301

### ethyl 5-(5-cyclopropyl-1,3-thiazol-4-yl)-1,3-oxazole-4-carboxylate

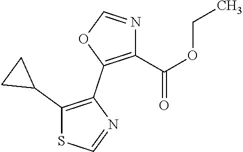 [SMILES: CCOC(=O)c1ncoc1-c1ncsc1C1CC1]

5-cyclopropyl-1,3-thiazole-4-carboxylic acid (2.00 g, 11.8 mmol) dissolved in 30 ml of THF was treated with 1,1,-carbonyl-diimidazole (2.30 g, 14.2 mmol) and stirred at room temperature. After 1 h, ethyl isocyanoacetate (1.4 ml, 13 mmol), dissolved in 20 ml of THF, and a solution of lithium bis(trimethylsilyl)amide in THF (12 ml, 1.0 M, 12 mmol) were added at 0° C. The mixture was allowed to warm to room temperature, stirred overnight and then concentrated in vacuo. The residue was taken up with ethyl acetate and washed with water. The organic layer was dried and concentrated in vacuo. The crude product was purified by column chromatography (50 g Ultra Snap Cartridge Biotage; eluent ethyl acetate/cyclohexane, elution gradient 10%→100%). Samples containing the desired product were united, the solvents were evaporated and the residue was dried in vacuum. 2.30 g (100% purity, 74% yield) of the title compound was used without further purification.

LC-MS (Method 7): Rₜ=1.37 min; MS (ESIpos): m/z=265 [M+H]⁺

¹H-NMR (600 MHz, DMSO-d6) δ [ppm]: 0.614 (1.40), 0.622 (5.17), 0.625 (4.35), 0.630 (4.44), 0.633 (5.10), 0.641 (1.42), 1.077 (1.50), 1.084 (4.03), 1.088 (4.16), 1.098 (4.20), 1.101 (4.01), 1.109 (1.32), 1.162 (7.86), 1.174 (16.00), 1.186 (7.91), 1.989 (2.00), 2.046 (0.62), 2.054 (1.27), 2.060 (1.37), 2.068 (2.39), 2.076 (1.30), 2.082 (1.16), 2.090 (0.56), 4.026 (0.49), 4.038 (0.48), 4.198 (2.59), 4.209 (7.79), 4.221 (7.66), 4.233 (2.49), 8.623 (9.20), 8.975 (8.85).

### Intermediate 302

### 2-amino-1-(5-cyclopropyl-1,3-thiazol-4-yl)ethanone hydrochloride

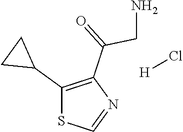

ethyl 5-(5-cyclopropyl-1,3-thiazol-4-yl)-1,3-oxazole-4-carboxylate (2.30 g, 8.70 mmol) was taken up in 43 ml of 6 N hydrochloric acid and stirred for 2 h at 100° C. The resulting mixture was concentrated in vacuo. 1.99 g (100% purity) of the title compound was obtained and used without further purification.

LC-MS (Method 9): Rₜ=1.15 min; MS (ESIpos): m/z=183 [M−HCl+H]⁺

### Intermediate 303

### tert-butyl [2-(5-cyclopropyl-1,3-thiazol-4-yl)-2-oxoethyl]carbamate

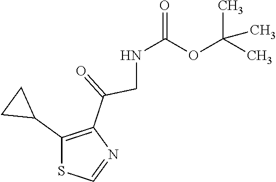 [SMILES: CC(C)(C)OC(=O)NCC(=O)c1ncsc1C1CC1]

To a solution of 2-amino-1-(5-cyclopropyl-1,3-thiazol-4-yl)ethanone hydrochloride (1.99 g, 9.10 mmol) in dichloromethane (41 ml) was added di-tert-butyl dicarbonate (2.18 g, 10.0 mmol) and triethylamine (7.6 ml, 55 mmol). After 2 h of stirring at RT, the reaction mixture was washed with water. After phase separation, the organic phase was dried; concentrated in vacuo and 2.44 g of the title compound was obtained and used without further purification.

LC-MS (Method 9): Rₜ=1.58 min; MS (ESIpos): m/z=283 [M+H]⁺

¹H-NMR (400 MHz, DMSO-d6) δ [ppm]: 0.724 (1.35), 0.729 (1.19), 0.741 (1.36), 0.752 (0.43), 1.109 (0.60), 1.268 (0.47), 1.279 (1.24), 1.285 (1.29), 1.300 (1.80), 1.306 (2.06), 1.316 (0.84), 1.364 (0.70), 1.388 (1.53), 1.402 (16.00), 1.467 (8.35), 3.129 (0.61), 4.394 (1.90), 4.409 (1.87), 6.976 (0.58), 8.892 (1.62).

### Intermediate 304

### rac-tert-butyl {[4-(5-cyclopropyl-1,3-thiazol-4-yl)-2,5-dioxoimidazolidin-4-yl]methyl}carbamate

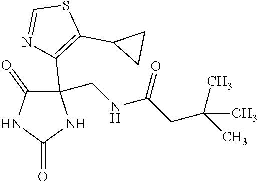 [SMILES: CC(C)(C)CC(=O)NCC1(c2ncsc2C2CC2)NC(=O)NC1=O]

To a solution of tert-butyl [2-(5-cyclopropyl-1,3-thiazol-4-yl)-2-oxoethyl]carbamate (2.44 g, 8.64 mmol) in methanol (20 ml) was added potassium cyanide (2.25 g, 34.6 mmol) and ammonium carbonate (3.32 g, 34.6 mmol) at RT. The reaction mixture was stirred for 5 hours at 60° C.; followed by 48 h at RT. into a sealed pressure flask. After additional 24 h stirring at 80° C., the reaction mixture was filtered at RT and the resulting filtrate was concentrated in vacuo.

The residue was purified by preparative HPLC (sample preparation: 5.6 g dissolved in acetonitrile; column: XBridge C18 5 μm, 100×30 mm; eluent:water→acetonitrile/water 80:20+1% ammonia solution→acetonitrile; flow rate: 80 ml/min; temperature: 40° C.; UV detection: 210 nm). 1.98 g of the desired product was obtained and used without further purification.

LC-MS (Method 7): Rₜ=1.22 min; MS (ESIpos): m/z=353 [M+H]⁺

### Intermediate 305

### rac-5-(aminomethyl)-5-(5-cyclopropyl-1,3-thiazol-4-yl)imidazolidine-2,4-dione hydrochloride

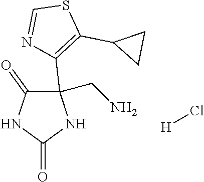 [SMILES: NCC1(c2ncsc2C2CC2)NC(=O)NC1=O ; Cl]

To a solution of rac-tert-butyl {[4-(5-cyclopropyl-1,3-thiazol-4-yl)-2,5-dioxoimidazolidin-4-yl]methyl}carbamate (500 mg, 1.42 mmol) in dichloromethane (12 ml) were added 1.8 ml of 4M hydrochloric acid (7.1 mmol) in dioxane at RT. The reaction mixture was stirred for 1 h at room temperature, after which extra portion of 4M hydrochloric acid (180 μl, 710 μmol) was added due to incomplete conversion. After stirring for 1 at RT, the precipitate was filtered off, washed with dichloromethane and dried in vacuo. 435 mg of the title compound was obtained and used without further purification.

LC-MS (Method 9): Rₜ=0.32 min; MS (ESIpos): m/z=253 [M−HCl+H]⁺

¹H-NMR (600 MHz, DMSO-d6) δ [ppm]: 0.005 (0.77), 0.553 (0.63), 0.562 (1.05), 0.570 (1.98), 0.576 (1.14), 0.578 (2.43), 0.584 (1.74), 0.587 (1.07), 0.593 (0.85), 0.719 (0.75), 0.725 (1.02), 0.728 (1.68), 0.734 (2.45), 0.742 (2.06), 0.751 (1.31), 0.759 (0.76), 1.014 (0.53), 1.021 (0.52), 1.024 (0.48), 1.029 (1.40), 1.036 (1.57), 1.039 (1.34), 1.045 (1.89), 1.049 (1.86), 1.054 (2.04), 1.058 (2.06), 1.063 (1.84), 1.068 (1.52), 1.071 (1.57), 1.078 (1.43), 1.083 (0.58), 1.086 (0.55), 1.093 (0.53), 1.596 (0.75), 1.910 (0.78), 1.919 (1.58), 1.924 (1.69), 1.927 (0.97), 1.932 (3.13), 1.938 (1.02), 1.941 (1.62), 1.946 (1.54), 1.955 (0.75), 2.523 (0.42), 2.573 (0.85), 3.058 (0.68), 3.168 (12.10), 3.568 (0.84), 3.615 (0.43), 3.626 (0.72), 3.637 (1.65), 3.647 (1.89), 3.653 (1.97), 3.662 (1.71), 3.684 (0.47), 8.368 (3.59), 8.628 (4.46), 8.862 (16.00), 11.327 (3.71).

### Intermediate 306

### methyl 5-(2,5-dimethyl-1,3-thiazol-4-yl)-1,3-oxazole-4-carboxylate

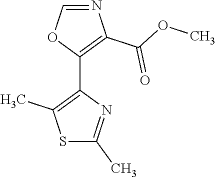

2,5-dimethyl-1,3-thiazole-4-carboxylicacid (3.00 g, 19.1 mmol) dissolved in 30 ml of THF was treated with 1,1,-carbonyl-diimidazole (3.71 g, 22.9 mmol) and stirred at room temperature. After 1 h, ethyl isocyanoacetate (2.3 ml, 21 mmol), dissolved in 30 ml of THF, and a solution of lithium bis(trimethylsilyl)amide in THF (19 ml, 1.0 M, 19 mmol) were added at 0° C. The mixture was allowed to warm to room temperature, stirred for 2 days and then concentrated in vacuo. The residue was taken up with ethyl acetate and washed with water. The organic layer was dried and concentrated in vacuo. The crude product (ethyl ester derivative) was diluted in 50 ml of methanol; Isolute® was added, and the solvent was evaporated on a rotary evaporator at higher temperature. The crude was then purified by column chromatography (340 g Ultra Snap Cartridge Biotage; eluent ethyl acetate/cyclohexane, elution gradient 15%→100%). Samples containing the desired product were united, the solvents were evaporated and the residue was dried in vacuum. 2.67 g (100% purity, 59% yield) of the title compound was used without further purification.

LC-MS (Method 7): Rₜ=1.11 min; MS (ESIpos): m/z=238 [M+H]⁺

¹H-NMR (400 MHz, DMSO-d6) δ [ppm]: 2.401 (13.72), 2.673 (12.80), 3.805 (16.00), 8.602 (2.93).

### Intermediate 307

### 2-amino-1-(2,5-dimethyl-1,3-thiazol-4-yl)ethanone hydrochloride

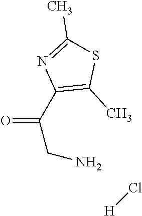

methyl 5-(2,5-dimethyl-1,3-thiazol-4-yl)-1,3-oxazole-4-carboxylate (2.67 g, 11.2 mmol) was taken up in 60 ml of 6 N hydrochloric acid and stirred for 2 h at 100° C. The resulting mixture was concentrated in vacuo. 2.90 g of the title compound was obtained and used without further purification.

LC-MS (Method 9): Rₜ=0.77 min; MS (ESIpos): m/z=171 [M+−HCl+H]⁺

### Intermediate 308

### tert-butyl [2-(2,5-dimethyl-1,3-thiazol-4-yl)-2-oxoethyl]carbamate

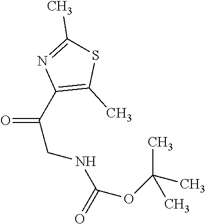 [SMILES: Cc1nc(C(=O)CNC(=O)OC(C)(C)C)c(C)s1]

To a solution of 2-amino-1-(2,5-dimethyl-1,3-thiazol-4-yl)ethanone hydrochloride (2.90 g, 14.0 mmol) in dichloromethane (80 ml) was added di-tert-butyl dicarbonate (2.86 g, 13.1 mmol) and triethylamine (13 ml, 95 mmol). After 2 h of stirring at RT, the reaction mixture was washed with water. After phase separation, the organic phase was dried; concentrated in vacuo and 3.17 g of the title compound was obtained and used without further purification.

LC-MS (Method 9): Rₜ=1.38 min; MS (ESIpos): m/z=271 [M+H]⁺

¹H-NMR (600 MHz, DMSO-d6) δ [ppm]: 1.111 (2.20), 1.275 (0.74), 1.367 (0.75), 1.394 (16.00), 1.412 (0.44), 2.500 (4.08), 2.607 (9.87), 2.663 (9.51), 4.143 (1.81), 4.153 (1.78), 7.156 (0.63).

### Intermediate 309

### rac-tert-butyl {[4-(2,5-dimethyl-1,3-thiazol-4-yl)-2,5-dioxoimidazolidin-4-yl]methyl}carbamate

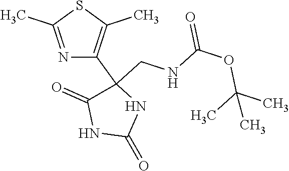 [SMILES: Cc1nc(C2(CNC(=O)OC(C)(C)C)NC(=O)NC2=O)c(C)s1]

To a solution of tert-butyl [2-(2,5-dimethyl-1,3-thiazol-4-yl)-2-oxoethyl]carbamate (3.17 g, 11.7 mmol) in methanol (21 ml) was added potassium cyanide (3.05 g, 46.9 mmol) and ammonium carbonate (4.51 g, 46.9 mmol) at RT. The reaction mixture was first stirred overnight at 60° C. into a sealed pressure flask. Extra portion of potassium cyanide (1.07 g, 16.4 mmol) and ammonium carbonate (1.58 g, 16.4 mmol) were added due to incomplete conversion. The reaction mixture was first stirred for 7 h at 60° C.; then for 48 h at RT.

After filtration and washing of the suspension with methanol at RT, the resulting filtrate was concentrated in vacuo. The residue was purified by preparative HPLC (sample preparation: 5.4 g dissolved in acetonitrile; column: XBridge C18 5 μm, 100×30 mm; eluent:water→acetonitrile/water 80:20+1% ammonia solution→acetonitrile; flow rate: 80 ml/min; temperature: 45° C.; UV detection: 210 nm). 1.22 g of the desired product was obtained and used without further purification.

LC-MS (Method 7): Rₜ=1.09 min; MS (ESIpos): m/z=341 [M+H]⁺

### Intermediate 310

### rac-5-(aminomethyl)-5-(2,5-dimethyl-1,3-thiazol-4-yl)imidazolidine-2,4-dione hydrochloride

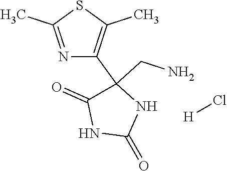 [SMILES: Cc1nc(C2(CN)NC(=O)NC2=O)c(C)s1 ; Cl]

To a solution rac-tert-butyl {[4-(2,5-dimethyl-1,3-thiazol-4-yl)-2,5-dioxoimidazolidin-4-yl]methyl}carbamate (300 mg, 881 μmol) in dichloromethane (3.5 ml) were added 1.1 ml of 4M hydrochloric acid (4.4 mmol) in dioxane at RT. After stirring for 1 at RT, the precipitate was filtered off, washed with dichloromethane and dried in vacuo. 243 mg of the title compound was obtained and used without further purification.

LC-MS (Method 9): Rₜ=0.24 min; MS (ESIpos): m/z=241 [M−HCl+H]⁺

¹H-NMR (400 MHz, DMSO-d₆) δ[ppm]: −0.008 (2.30), 0.008 (2.40), 1.110 (0.56), 1.596 (1.42), 1.757 (2.42), 2.074 (2.42), 2.273 (0.40), 2.368 (0.52), 2.418 (12.62), 2.525 (1.42), 2.567 (16.00), 2.712 (0.47), 3.168 (8.55), 3.403 (0.64), 3.568 (1.07), 3.579 (0.83), 3.600 (0.74), 8.432 (1.64), 8.931 (0.84), 11.379 (2.14).

### Intermediate 311

### ethyl 5-(1-chlorocyclopropyl)-1,3-oxazole-4-carboxylate

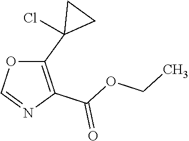

1-chlorocyclopropane-1-carboxylic acid (4.12 g, 34.2 mmol) dissolved in 50 ml of THF was treated with 1,1,-carbonyl-diimidazole (6.65 g, 41.0 mmol) and stirred at room temperature. After 2 h, ethyl isocyanoacetate (4.1 ml, 38 mmol), dissolved in 50 ml of THF, and a solution of lithium bis(trimethylsilyl)amide in THF (34 ml, 1.0 M, 34 mmol) were added at 0° C. The mixture was allowed to warm to room temperature, stirred overnight and then concentrated in vacuo. The residue was taken up with ethyl acetate and washed with water. The organic layer was dried and concentrated in vacuo. The crude product was purified by column chromatography (340 g Ultra Snap Cartridge Biotage; eluent ethyl acetate/cyclohexane, elution gradient 10%→100%). Samples containing the desired product were united, the solvents were evaporated and the residue was dried in vacuum. 3.38 g of the title compound was used without further purification.

LC-MS (Method 7): Rₜ=1.50 min; MS (ESIpos): m/z=216 [M+H]⁺

¹H-NMR (400 MHz, DMSO-d6) δ [ppm]: −0.008 (0.64), 0.008 (0.67), 1.314 (7.63), 1.332 (16.00), 1.350 (7.76), 1.533 (3.46), 1.540 (13.18), 1.543 (13.20), 1.550 (3.34), 2.524 (0.43), 4.298 (2.49), 4.315 (7.69), 4.333 (7.55), 4.351 (2.42), 8.489 (5.44).

### Intermediate 312

### 2-amino-1-(1-chlorocyclopropyl)ethanone hydrochloride

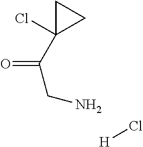

ethyl 5-(1-chlorocyclopropyl)-1,3-oxazole-4-carboxylate (3.38 g, 15.7 mmol) was taken up in 77 ml of 6 N hydrochloric acid and stirred for 2 h at 100° C. The resulting mixture was concentrated in vacuo. 2.56 g of the title compound was obtained and used without further purification.

LC-MS (Method 9): Rₜ=0.82 min; MS (ESIpos): m/z=134 [M−HCl+H]⁺

¹H-NMR (600 MHz, DMSO-d₆) δ[ppm]: 1.253 (0.53), 1.574 (4.17), 1.583 (12.73), 1.589 (12.55), 1.597 (5.57), 1.625 (0.64), 1.721 (0.61), 1.749 (5.48), 1.758 (12.77), 1.763 (12.59), 1.773 (4.38), 3.652 (0.41), 4.187 (16.00), 7.297 (1.22), 7.381 (1.40), 7.466 (1.22), 8.403 (5.29).

### Intermediate 313

### tert-butyl [2-(1-chlorocyclopropyl)-2-oxoethyl]carbamate

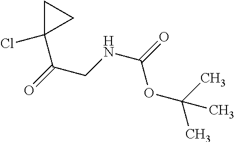

To a solution of 2-amino-1-(1-chlorocyclopropyl)ethanone hydrochloride (2.56 g, 15.1 mmol) in dichloromethane (100 ml) was added di-tert-butyl dicarbonate (3.61 g, 16.6 mmol) and triethylamine (17 ml, 120 mmol). After 2 h of stirring at RT, the reaction mixture was washed with water. After phase separation, the organic phase was dried; concentrated in vacuo and 3.22 g of the title compound was obtained and used without further purification.

¹H-NMR (600 MHz, DMSO-d6) δ [ppm]: 1.015 (0.44), 1.113 (1.18), 1.327 (0.64), 1.340 (0.71), 1.368 (4.07), 1.377 (9.45), 1.390 (0.93), 1.425 (0.51), 1.449 (0.50), 1.458 (1.39), 1.463 (1.83), 1.470 (16.00), 1.631 (0.53), 1.639 (1.01), 1.644 (0.90), 2.501 (1.09), 4.091 (1.08), 4.101 (1.07).

### Intermediate 314

### rac-tert-butyl {[4-(1-chlorocyclopropyl)-2,5-dioxoimidazolidin-4-yl]methyl}carbamate

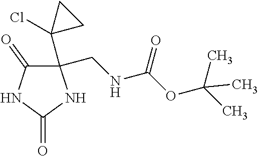 [SMILES: CC(C)(C)OC(=O)NCC1(C2(Cl)CC2)NC(=O)NC1=O]

To a solution of tert-butyl [2-(1-chlorocyclopropyl)-2-oxoethyl]carbamate (3.20 g, 13.7 mmol) in methanol (12 ml) was added potassium cyanide (3.57 g, 54.8 mmol) and ammonium carbonate (5.26 g, 54.8 mmol) at RT. The reaction mixture was stirred for 6 hours at 80° C.; followed by 48 h at 40° C. into a sealed pressure flask. After filtration and washing of the suspension with methanol at RT, the resulting filtrate was concentrated in vacuo. The residue was purified by preparative HPLC (sample preparation: 5.14 g dissolved in acetonitrile; column: XBridge C18 5 μm, 100×30 mm; eluent:water→acetonitrile/water 80:20+1% ammonia solution→acetonitrile; flow rate: 80 ml/min; temperature: 40° C.; UV detection: 210 nm). 342 mg of the desired product was obtained and used without further purification.

LC-MS (Method 9): Rₜ=0.72 min; MS (ESIneg): m/z=302 [M−H]⁻

¹H-NMR (400 MHz, DMSO-d6) δ [ppm]: 1.007 (0.66), 1.025 (0.99), 1.153 (0.56), 1.165 (1.09), 1.362 (16.00), 1.409 (0.62), 7.833 (0.77).

### Intermediate 315

### rac-5-(aminomethyl)-5-(1-chlorocyclopropyl)imidazolidine-2,4-dione hydrochloride

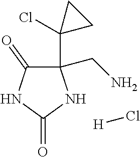

To a solution of rac-tert-butyl {[4-(1-chlorocyclopropyl)-2,5-dioxoimidazolidin-4-yl]methyl}carbamate (340 mg, 1.12 mmol) in dichloromethane (10 ml) were added 1.4 ml of 4M hydrochloric acid (5.6 mmol) in dioxane at RT. The reaction mixture was stirred for 2 h at room temperature and concentrated in vacuo. The crude was again diluted in 10 ml DCM after which extra portion of 4M hydrochloric acid (1.4 ml, 4.0 M, 5.6 mmol) was added due to incomplete conversion. After stirring overnight at RT, the precipitate was filtered off, washed with dichloromethane and dried in vacuo. 256 mg of the title compound was obtained and used without further purification.

LC-MS (Method 9): Rₜ=0.27 min; MS (ESIpos): m/z=204 [M−HCl+H]⁺

### Intermediate 316

### Ethyl (2E)-2-[2-(4-chloro-2-fluorophenyl)hydrazinylidene]-3-oxopropanoate

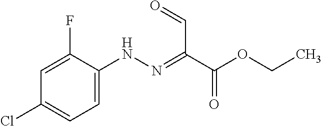 [SMILES: CCOC(=O)/C(C=O)=N/Nc1ccc(Cl)cc1F]

4-chloro-2-fluoroaniline (190 μl, 1.7 mmol) was dissolved in 2.5 ml of water and 470 μl of concentrated hydrochloric acid. The mixture was cooled to 0° C. and a solution of sodium nitrite (118 mg, 1.72 mmol) in 1.25 ml of water was added dropwise. After stirring for 5 minutes this solution was added dropwise to a solution of ethyl-(2E)-3-(dimethylamino)acrylate (270 μl, 1.9 mmol) and potassium acetate (253 mg, 2.58 mmol) in 3.75 ml of ethanol at 0° C. The mixture was stirred at room temperature for 30 min before being diluted with ethyl acetate and washed twice with water and once with brine. The organic layer was dried over sodium sulfate, filtered and evaporated. 414 mg (78% purity, 69% yield) of the title compound were obtained.

LC-MS (Method 7): Rₜ=2.13 min; MS (ESIneg): m/z=271 [M−H]⁻

### Intermediate 317

### Ethyl (2E,3E)-2-[2-(4-chloro-2-fluorophenyl)hydrazinylidene]-3-(hydroxyimino)propanoate

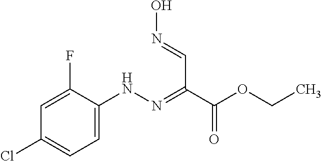 [SMILES: CCOC(=O)C(/C=N/O)=N/Nc1ccc(Cl)cc1F]

Ethyl (2E)-2-[2-(4-chloro-2-fluorophenyl)hydrazinylidene]-3-oxopropanoate (404 mg, 78% purity, 1.16 mmol), hydroxylamine hydrochloride (96.4 mg, 1.39 mmol) and potassium acetate (284 mg, 2.89 mmol) were dissolved in 7.0 ml of ethanol and the mixture was stirred at room temperature over night. The reaction mixture was diluted with ethyl acetate and washed twice with water and once with brine. The organic layer was dried over sodium sulfate, filtered and evaporated. The residue was dried in vacuum. 349 mg (77% purity, 81% yield) of the title compound were obtained.

LC-MS (Method 7): Rₜ=2.00 min; MS (ESIpos): m/z=288 [M+H]⁺

### Intermediate 318

### Ethyl 2-(4-chloro-2-fluorophenyl)-2H-1,2,3-triazole-4-carboxylate

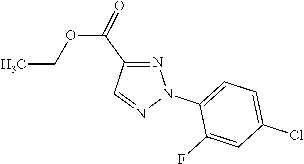 [SMILES: CCOC(=O)c1cnn(-c2ccc(Cl)cc2F)n1]

Ethyl (2E,3E)-2-[2-(4-chloro-2-fluorophenyl)hydrazinylidene]-3-(hydroxyimino)propanoate (348 mg, 77% purity, 931 μmol) was stirred in 3.5 ml of acetic acid anhydride at 60° C. over night. The reaction was then diluted in ethyl acetate and washed twice with water and once with brine. The organic layer was dried over sodium sulfate, filtered and evaporated together with Isolute®. The residue was purified by chromatography on silica gel (25 g Ultra Snap Cartridge Biotage®;

Biotage-Isolera-One®; Cy/EE-gradient: 2% EE-20% EE; flow: 75 ml/min). Samples containing the desired product were united, the solvents were evaporated and the residue was dried in vacuum. 173 mg (85% purity, 59% yield) of the title compound were obtained.

LC-MS (Method 7): Rₜ=2.02 min; MS (ESIpos): m/z=270 [M+H]⁺

### Intermediate 319

### 2-(4-chloro-2-fluorophenyl)-2H-1,2,3-triazole-4-carboxylic acid

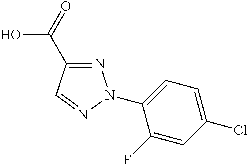 [SMILES: O=C(O)c1cnn(-c2ccc(Cl)cc2F)n1]

Ethyl 2-(4-chloro-2-fluorophenyl)-2H-1,2,3-triazole-4-carboxylate (173 mg, 85% purity, 549 μmol) was dissolved in 12 ml of THF, treated with lithium hydroxide solution (550 μl, 1.0 M, 550 μmol) and the mixture was stirred for 2 h at room temperature. More lithium hydroxide solution (550 μl, 1.0 M, 550 μmol) was added and the mixture was stirred 1 h at room temperature. The solvent was removed on a rotary evaporator. Water was added and the mixture was acidified with 2 N hydrochloric acid. The resulting precipitate was collected by filtration. 138 mg (95% purity, 98% yield) of the title compound were obtained.

LC-MS (Method 7): Rₜ=1.36 min; MS (ESIpos): m/z=242 [M+H]⁺

### Intermediate 320

### Ethyl (2E)-2-[2-(2,4-dichlorophenyl)hydrazinylidene]-3-oxopropanoate

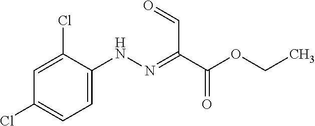 [SMILES: CCOC(=O)/C(C=O)=N/Nc1ccc(Cl)cc1Cl]

2,4-dichloroaniline (250 mg, 1.52 mmol) was dissolved in 2.5 ml of water and 470 μl of concentrated hydrochloric acid. The mixture was cooled to 0° C. and a solution of sodium nitrite (105 mg, 1.52 mmol) in 1.25 ml of water was added dropwise. After stirring for 5 minutes this solution was added dropwise to a solution of ethyl-(2E)-3-(dimethylamino)acrylate (240 μl, 1.7 mmol) and potassium acetate (224 mg, 2.29 mmol) in 3.75 ml of ethanol at 0° C. The mixture was stirred at room temperature for 30 min before being diluted with ethyl acetate and washed twice with water and once with brine. The organic layer was dried over sodium sulfate, filtered and evaporated. 264 mg (54% purity, 33% yield) of the title compound were obtained.

LC-MS (Method 9): Rₜ=1.96 min; MS (ESIpos): m/z=289 [M+H]⁺

### Intermediate 321

### Ethyl (2E,3E)-2-[2-(2,4-dichlorophenyl)hydrazinylidene]-3-(hydroxyimino)propanoate

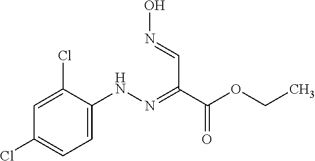 [SMILES: CCOC(=O)C(/C=N/O)=N/Nc1ccc(Cl)cc1Cl]

Ethyl (2E)-2-[2-(2,4-dichlorophenyl)hydrazinylidene]-3-oxopropanoate (264 mg, 54% purity, 495 μmol), hydroxylamine hydrochloride (41.3 mg, 595 μmol) and potassium acetate (122 mg, 1.24 mmol) were dissolved in 3 ml of ethanol and the mixture was stirred at room temperature over night. The reaction mixture was diluted with ethyl acetate and washed twice with water and once with brine. The organic layer was dried over sodium sulfate, filtered and evaporated. The residue was dried in vacuum. 239 mg (89% purity, 141% yield) of the title compound were obtained.

LC-MS (Method 7): Rₜ=2.11 min; MS (ESIpos): m/z=304 [M+H]⁺

### Intermediate 322

### Ethyl 2-(2,4-dichlorophenyl)-2H-1,2,3-triazole-4-carboxylate

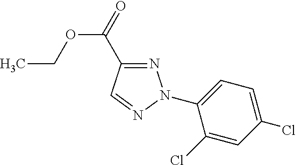 [SMILES: CCOC(=O)c1cnn(-c2ccc(Cl)cc2Cl)n1]

Ethyl (2E,3E)-2-[2-(2,4-dichlorophenyl)hydrazinylidene]-3-(hydroxyimino)propanoate (238 mg, 89% purity, 696 μmol) was stirred in 4 ml of acetic acid anhydride at 60° C. over night. The reaction was then diluted in ethyl acetate and washed twice with water and once with brine. The organic layer was dried over sodium sulfate, filtered and evaporated together with Isolute®. The residue was purified by chromatography on silica gel (25 g Ultra Snap Cartridge Biotage®; Biotage-Isolera-One®; Cy/EE-gradient: 2% EE-20% EE; flow: 75 ml/min). Samples containing the desired product were united, the solvents were evaporated and the residue was dried in vacuum. 118 mg (75% purity, 44% yield) of the title compound were obtained.

LC-MS (Method 7): Rₜ=2.15 min; MS (ESIpos): m/z=286 [M+H]⁺

### Intermediate 323

### 2-(2,4-dichlorophenyl)-2H-1,2,3-triazole-4-carboxylic acid

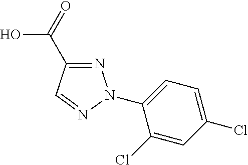 [SMILES: O=C(O)c1cnn(-c2ccc(Cl)cc2Cl)n1]

Ethyl 2-(2,4-dichlorophenyl)-2H-1,2,3-triazole-4-carboxylate (117 mg, 75% purity, 307 μmol) was dissolved in 5 ml of THF, treated with lithium hydroxide solution (310 μl, 1.0 M, 310 μmol) and the mixture was stirred for 1 h at room temperature. The solvent was removed on a rotary evaporator. Water was added an the mixture was acidified with 2 N hydrochloric acid. The resulting precipitate was collected by filtration. 86.0 mg (100% purity, 109% yield) of the title compound were obtained.

LC-MS (Method 7): Rₜ=1.52 min; MS (ESIneg): m/z=255 [M−H]⁻

### Intermediate 324

### Ethyl 5-ethyl-1,3-oxazole-4-carboxylate

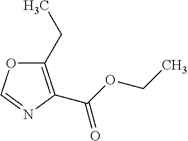

Propanoic acid (1.00 g, 13.5 mmol) dissolved in 15 ml of THF was treated with 1,1,-carbonyl-diimidazole (2.63 g, 16.2 mmol) and stirred at room temperature. After 2 h ethyl isocyanoacetate (1.6 ml, 15 mmol), dissolved in 10 ml of THF, and a solution of lithium bis(trimethylsilyl)amide in THF (13 ml, 1.0 M, 13 mmol) were added at 0° C. The mixture was allowed to warm to room temperature and stirred over night. The solvent was removed on a rotary evaporator. The residue was taken up with ethyl acetate and washed with water and saturated aqueous sodium chloride solution. The organic layer was dried over sodium sulfate, filtered, Isolute® was added and the solvent was evaporated on a rotary evaporator. The crude product was purified by column chromatography (Machine: Biotage® Isolera One; column: Biotage® SNAP Ultra 50 g; gradient: Cy/EE-gradient, 8% EE-80% EE; flow: 100 ml/min). Product containing samples were united and the solvents were removed on a rotary evaporator. 1.40 g (97% purity, 59% yield) of the title compound were obtained.

LC-MS (Method 7): Rₜ=1.30 min; MS (ESIpos): m/z=170 [M+H]⁺

### Intermediate 325

### 1-aminobutan-2-one hydrogen chloride

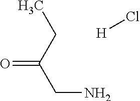

Ethyl 5-ethyl-1,3-oxazole-4-carboxylate (1.40 g, 8.28 mmol) was taken up in 28 ml of 6 N hydrochloric acid and stirred at 100° C. After 4 h the solvent was removed on a rotary evaporator and the residue was dried in vacuo. 0.99 g (97% yield) of the title compound were obtained.

### Intermediate 326

### tert-butyl (2-oxobutyl)carbamate

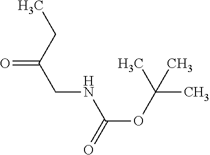

1-aminobutan-2-one-hydrogen chloride (990 mg, 8.01 mmol) dissolved in 30 ml of dichloromethane was treated with di-tert-butyl dicarbonate (2.0 ml, 8.8 mmol) and triethylamine (3.3 ml, 24 mmol). The mixture was stirred at room temperature over night. The solvent was removed on a rotary evaporator. The residue was taken up with ethyl acetate and washed with water and saturated aqueous sodium chloride solution. The organic layer was dried over sodium sulfate, filtered and evaporated on a rotary evaporator. 1.2 g (80% yield) of the title compound were obtained.

### Intermediate 327

### rac-tert-butyl [(4-ethyl-2,5-dioxoimidazolidin-4-yl)methyl]carbamate

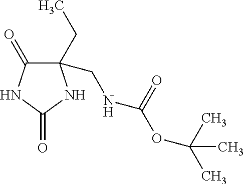 [SMILES: CCC1(CNC(=O)OC(C)(C)C)NC(=O)NC1=O]

Ammonium carbonate (6.16 g, 64.1 mmol) and ammonium chloride (1.37 g, 25.6 mmol) were dissolved in 10 ml of water. Tert-butyl (2-oxobutyl)carbamate (1.20 g, 6.41 mmol), dissolved in 10 ml of ethanol was added and the mixture was stirred for 15 min at room temperature. Then potassium cyanide (1.88 g, 28.8 mmol) was added, the vial was sealed and the mixture was stirred at 80° C. over night. The solvent was removed on a rotary evaporator. The residue was taken up with ethyl acetate and washed with water and saturated aqueous sodium chloride solution. The organic layer was dried over sodium sulfate, filtered and concentrated on a rotary evaporator. 1.17 g (100% purity, 71% yield) of the title compound were obtained.

LC-MS (Method 7): Rₜ=0.96 min; MS (ESIneg): m/z=256 [M−H]⁻

### Intermediate 328

### rac-5-(aminomethyl)-5-ethylimidazolidine-2,4-dione hydrochloride

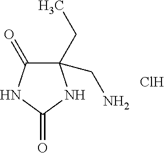

rac-tert-butyl [(4-ethyl-2,5-dioxoimidazolidin-4-yl)methyl]carbamate (1.17 g, 4.55 mmol) was dissolved in 26 ml of dichloromethane. 4 M hydrochloric acid in 1,4-dioxane (5.7 ml, 4.0 M, 23 mmol) was added and the mixture was stirred for 2 h. The solvent was removed on a rotary evaporator and the residue was taken up in dichloromethane. The solvent was removed and the residue was dried in vacuo. 1.00 g (95% purity, 108% yield) of the title compound were obtained.

LC-MS (Method 9): Rₜ=0.25 min; MS (ESIpos): m/z=158 [M+H-HCl]⁺

### Intermediate 329

### ent-tert-butyl {[4-(1-methyl-1H-imidazol-2-yl)-2,5-dioxoimidazolidin-4-yl]methyl}carbamate

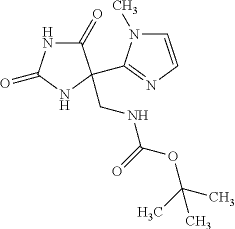 [SMILES: Cn1ccnc1C1(CNC(=O)OC(C)(C)C)NC(=O)NC1=O]

To a solution of tert-butyl [2-(1-methyl-1H-imidazol-2-yl)-2-oxoethyl]carbamate (4.96 g, 20.7 mmol, Example 3d in J: Med. Chem. 2014, 57, 10476-10485) in a mixture of Ethanol (30 ml) and water (12 ml) was added potassium cyanide (5.40 g, 82.9 mmol) and ammonium carbonate (7.97 g, 82.9 mmol) at RT. The reaction mixture was heated up to 80° C. for 2 days into a sealed pressure flask. The resulting mixture was diluted with water, partially concentrated in vacuo and filtered. The resulting filtrate was concentrated in vacuo and 10.78 g of the crude product were isolated. 9.3 g of this crude product were suspended in ethanol and the residue was filtered off and washed 3 times with ethanol. The combined filtrate was concentrated in vacuo and the resulting product was purified by preparative chiral SFC (sample preparation: 7.3 g dissolved in a mixture of methanol and acetonitrile; column: Chirapak AD-H (SFC) 5 μm, 250×30 mm; eluent: carbon dioxide/methanol 78:22; flow rate: 125 ml/min; temperature: 40° C.; UV detection: 210 nm). 2 g (6.47 mmol) of the desired product was obtained

Analytical chiral SFC: Rₜ=1.70 min, e.e. =99% [column: AD 3 μm, 100×4.6 mm; eluent: carbon dioxide/methanol 80:20; flow rate: 3 ml/min; temperature: 40° C.; UV detection: 210 nm]

¹H NMR (600 MHz, DMSO-d₆) b ppm 11.09 (bs, 1H), 8.16 (s, 1H), 7.20 (s, 1H), 6.67-6.95 (m, 2H), 3.73-3.91 (m, 2H), 3.49 (s, 3H), 1.37 (s, 9H).

The title compound can also be synthesized via the procedure described in J: Med. Chem. 2014, 57, 10476-10485

### Intermediate 330

### ent-5-(aminomethyl)-5-(1-methyl-1H-imidazol-2-yl)imidazolidine-2,4-dione hydrochloride

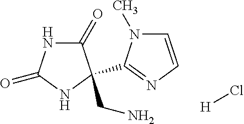

To a solution of ent-tert-butyl {[4-(1-methyl-1H-imidazol-2-yl)-2,5-dioxoimidazolidin-4-yl]methyl}carbamate (2.00 g, 6.46 mmol) in methanol (46 ml) were added 16 ml (65 mmol) of 4M hydrochloric acid in dioxane at 0° C. The resulting suspension was stirred overnight at room temperature. After filtration, the filtrate was concentrated in vacuo (1.23 g of the title compound) and the remaining residue was washed twice with methanol and dried under vacuum (0.41 g of of the title compound). Both fractions were used in the next step without further purification.

LC-MS (Method 9): Rₜ=0.23 min; MS (ESIpos): m/z=210 [M−HCl+H]⁺

¹H-NMR (600 MHz, DMSO-d6) δ [ppm]: 2.389 (0.48), 2.428 (0.49), 3.061 (1.66), 3.166 (1.50), 3.296 (0.55), 3.388 (1.15), 3.494 (0.46), 3.510 (0.45), 3.666 (0.48), 3.677 (0.51), 3.702 (0.83), 3.738 (6.54), 3.782 (16.00), 3.846 (2.02), 3.895 (1.13), 3.910 (1.64), 3.924 (0.90), 3.929 (0.64), 3.988 (0.48), 4.021 (0.52), 4.050 (0.72), 4.080 (0.42), 4.119 (0.80), 4.129 (0.89), 4.160 (0.45), 4.259 (0.45), 4.697 (0.54), 7.250 (1.55), 7.322 (2.88), 7.335 (2.41), 7.419 (1.52), 7.545 (5.13), 7.729 (0.53), 7.776 (0.43), 8.699 (4.41), 9.125 (3.30), 11.735 (5.00).

### Experimental Section—Examples

General Method a for the Synthesis of Hydantoin Amides 1-27

The suitable carboxylic acid (1.0 eq.) and the corresponding amine (1.1 eq.) were dissolved in the appropriate amount of N,N-dimethylformamide and 1-(3-dimethylaminopropyl)-3-ethylcarbodiimide hydrochloride (3.0 eq.), 1-hydroxybenzotriazole (1.3 eq.) or 1-hydroxy-7-azabenzotriazole (1.3 eq., 1M in N,N-dimethylacetamide) and N,N-diisopropylethylamine (2.8 eq.) or trimethylamine (2.8 eq.) were added allowing the mixture to stir at room temperature for 16 hours. After reaction's completion, the crude was dissolved in ethyl acetate and sequentially extracted with 1N hydrochloric acid and sodium hydrogencarbonate. The organic layer was dried over magnesium sulfate, filtered and concentrated in vacuo to give the crude product, which was purified either by precipitation with diethyl ether and heptane or by column chromatography in a suitable mixture of solvents.

### Example 1

### N-{[(4R)-4-cyclopropyl-2,5-dioxoimidazolidin-4-yl]methyl}-2-(3-fluorophenyl)-2H-1,2,3-triazole-4-carboxamide

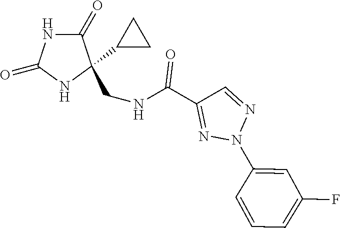 [SMILES: O=C1NC(=O)[C@](CNC(=O)c2cnn(-c3cccc(F)c3)n2)(C2CC2)N1]

The compound was synthesized according to the General Method A using 92 mg (0.44 mmol) of 2-(3-fluorophenyl)-2H-1,2,3-triazole-4-carboxylic acid, 100 mg (0.48 mmol) of (5R)-5-(aminomethyl)-5-cyclopropylimidazolidine-2,4-dione hydrochloride, 110 mg (0.57 mmol) of 1-(3-dimethylaminopropyl)-3-ethylcarbodiimide hydrochloride, 88 mg (0.57 mmol) of 1-hydroxybenzotriazole and 0.22 mL (1.20 mmol) of N,N-diisopropylethylamine in 3 mL of N,N-dimethylformamide. The crude product obtained from the workup was precipitated with diethyl ether and heptane to give 67 mg (41% yield, 97% purity) of the pure compound as an off-white powder.

¹H-NMR (300 MHz, DMSO-d₆): δ [ppm]=0.06-0.19 (m, 1H), 0.23-0.46 (m, 3H), 1.08-1.19 (m, 1H), 3.66 (ddd, 2H), 7.29 (bp, 1H), 7.36 (t, 1H), 7.68 (q, 1H), 7.83-7.96 (m, 2H), 8.47-8.58 (m, 2H)

LC-MS (Method 1): Rₜ=2.496 min. MS (Mass method 1): m/z=359 (M+H)⁺

### Example 2

### rac-N-{[4-tert-butyl-2,5-dioxoimidazolidin-4-yl]methyl}-2-[3-fluoro-4-(trifluoromethyl)phenyl]-2H-1,2,3-triazole-4-carboxamide

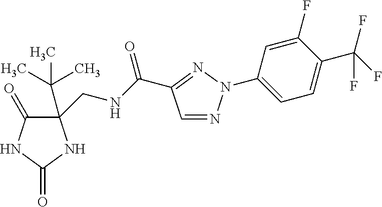 [SMILES: CC(C)(C)C1(CNC(=O)c2cnn(-c3ccc(C(F)(F)F)c(F)c3)n2)NC(=O)NC1=O]

The compound was synthesized according to the General Method A using 150 mg (0.54 mmol) of 2-[3-fluoro-4-(trifluoromethyl)phenyl]-2H-1,2,3-triazole-4-carboxylic acid, 101 mg (0.54 mmol) of rac-5-(aminomethyl)-5-tert-butylimidazolidine-2,4-dione, 157 mg (0.54 mmol) of 1-(3-dimethylaminopropyl)-3-ethylcarbodiimide hydrochloride, 0.82 mL (0.82 mmol) of 1M 1-hydroxy-7-azabenzotriazole in N,N-dimethylacetamide and 0.23 mL (1.60 mmol) of triethylamine in 3 mL of N,N-dimethylformamide. The crude product obtained from the workup was purified by flash column chromatography on silica gel eluting with dichloromethane/methanol mixtures to afford 100 mg (41% yield, 99% purity) of the racemic product as a white powder.

¹H-NMR (300 MHz, DMSO-d₆): δ [ppm]=1.03 (s, 9H), 3.75 (d, 2H), 7.64 (s, 1H), 8.09 (bp, 2H), 8.16 (d, 1H), 8.51 (t, 1H), 8.58 (s, 1H), 10.61 (s, 1H).

LC-MS (Method 1): Rₜ=3.186 min. MS (Mass method 1): m/z=443 (M+H)⁺

### Example 3

### ent-N-{[4-tert-butyl-2,5-dioxoimidazolidin-4-yl]methyl}-2-(4-fluorophenyl)-2H-1,2,3-triazole-4-carboxamide

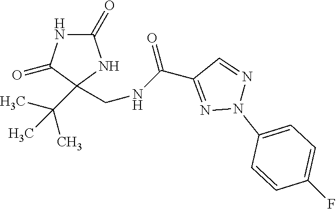 [SMILES: CC(C)(C)C1(CNC(=O)c2cnn(-c3ccc(F)cc3)n2)NC(=O)NC1=O]

The compound was synthesized according to the General Method A using 76 mg (0.37 mmol) of 2-(4-fluorophenyl)-2H-1,2,3-triazole-4-carboxylic acid, 75 mg (0.40 mmol) of rac-5-(aminomethyl)-5-tert-butylimidazolidine-2,4-dione, 92 mg (0.48 mmol) of 1-(3-dimethylaminopropyl)-3-ethylcarbodiimide hydrochloride, 73 mg (0.48 mmol) of 1-hydroxybenzotriazole and 0.18 mL (1.00 mmol) of N,N-diisopropylethylamine in 3 mL of N,N-dimethylformamide. The crude product obtained from the workup was precipitated with diethyl ether and heptane to give the pure racemic compound. The racemate was resolved using a chiral column with a gradient from 75% n-heptane/25% ethanol to 100% ethanol, to give 3 mg (2% yield, 96% purity) of the enantiopure product as a white powder.

¹H-NMR (300 MHz, DMSO-d₆): δ [ppm]=1.01 (s, 9H), 3.73 (ddd, 2H), 7.48 (t, 2H), 7.61 (s, 1H), 8.07 (dd, 2H), 8.31 (t, 1H), 8.45 (s, 1H), 10.60 (s, 1H).

LC-MS (Method 1): Rₜ=2.648 min. MS (Mass method 1): m/z=375 (M+H)⁺

### Example 4

### rac-N-{[4-tert-butyl-2,5-dioxoimidazolidin-4-yl]methyl}-2-(3-fluorophenyl)-2H-1,2,3-triazole-4-carboxamide

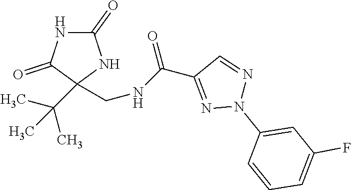 [SMILES: CC(C)(C)C1(CNC(=O)c2cnn(-c3cccc(F)c3)n2)NC(=O)NC1=O]

The compound was synthesized according to the General Method A using 76 mg (0.37 mmol) of 2-(3-fluorophenyl)-2H-1,2,3-triazole-4-carboxylic acid, 75 mg (0.40 mmol) of rac-5-(aminomethyl)-5-tert-butylimidazolidine-2,4-dione, 92 mg (0.48 mmol) of 1-(3-dimethylaminopropyl)-3-ethylcarbodiimide hydrochloride, 73 mg (0.48 mmol) of 1-hydroxybenzotriazole and 0.18 mL (1.00 mmol) of N,N-diisopropylethylamine in 3 mL of N,N-dimethylformamide. The crude product obtained from the workup was precipitated with diethyl ether and heptane to give 5 mg (3% yield, 99% purity) of the racemic product as a white powder.

¹H-NMR (300 MHz, DMSO-d₆): δ [ppm]=1.03 (s, 9H), 3.74 (ddd, 2H), 7.38 (t, 1H), 7.61 (s, 1H), 7.69 (dd, 1H), 7.83-7.96 (m, 2H), 8.38 (t, 1H), 8.49 (s, 1H), 10.61 (bp, 1H).

LC-MS (Method 1): Rₜ=2.687 min. MS (Mass method 1): m/z=375 (M+H)⁺

### Example 5

### N-{[(4R)-4-cyclopropyl-2,5-dioxoimidazolidin-4-yl]methyl}-2-[3-fluoro-4-(trifluoromethyl)phenyl]-2H-1,2,3-triazole-4-carboxamide

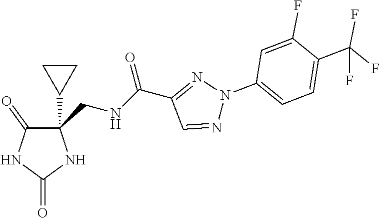 [SMILES: O=C1NC(=O)[C@](CNC(=O)c2cnn(-c3ccc(C(F)(F)F)c(F)c3)n2)(C2CC2)N1]

The compound was synthesized according to the General Method A using 100 mg (0.36 mmol) of 2-[3-fluoro-4-(trifluoromethyl)phenyl]-2H-1,2,3-triazole-4-carboxylic acid, 75 mg (0.36 mmol) of (5R)-5-(aminomethyl)-5-cyclopropylimidazolidine-2,4-dione hydrochloride, 105 mg (0.54 mmol) of 1-(3-dimethylaminopropyl)-3-ethylcarbodiimide hydrochloride, 0.55 mL (0.55 mmol) of 1M 1-hydroxy-7-azabenzotriazole in N,N-dimethylacetamide and 0.15 mL (1.10 mmol) of triethylamine in 3 mL of N,N-dimethylformamide. The crude product obtained from the workup was purified by flash column chromatography on silica gel eluting with dichloromethane/methanol mixtures to afford 78 mg (49% yield, 98% purity) of the product as a white powder.

¹H-NMR (300 MHz, DMSO-d₆): δ [ppm]=0.07-0.20 (m, 1H), 0.28-0.51 (m, 3H), 1.11-1.22 (m, 1H), 3.71 (ddd, 2H), 7.64 (s, 1H), 803-8.14 (m, 2H), 8.17 (d, 1H), 8.59 (s, 1H), 8.64 (t, 1H), 10.65 (b ID, 1H).

LC-MS (Method 1): Rₜ=3.026 min. MS (Mass method 1): m/z=427 (M+H)⁺

### Example 6

### rac-N-[(4-tert-butyl-2,5-dioxoimidazolidin-4-yl)methyl]-2-[4-(trifluoromethyl)phenyl]-2H-1,2,3-triazole-4-carboxamide

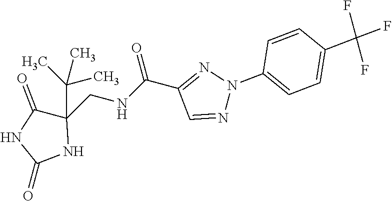 [SMILES: CC(C)(C)C1(CNC(=O)c2cnn(-c3ccc(C(F)(F)F)cc3)n2)NC(=O)NC1=O]

The compound was synthesized according to the General Method A using 115 mg (0.45 mmol) of 2-[4-(trifluoromethyl)phenyl]-2H-1,2,3-triazole-4-carboxylic acid, 83 mg (0.45 mmol) of rac-5-(aminomethyl)-5-tert-butylimidazolidine-2,4-dione, 129 mg (0.67 mmol) of 1-(3-dimethylaminopropyl)-3-ethylcarbodiimide hydrochloride, 0.67 mL (0.67 mmol) of 1M 1-hydroxy-7-azabenzotriazole in N,N-dimethylacetamide and 0.19 mL (1.30 mmol) of triethylamine in 3 mL of N,N-dimethylformamide. The crude product obtained from the workup was purified by flash column chromatography on silica gel eluting with dichloromethane/methanol mixtures to afford 35 mg (18% yield, 99% purity) of the racemic product as a white powder.

¹H-NMR (300 MHz, DMSO-d₆): δ [ppm]=1.03 (s, 9H), 3.75 (ddd, 2H), 7.62 (s, 1H), 8.03 (d, 2H), 8.28 (d, 2H), 8.44 (t, 1H), 8.54 (s, 1H), 10.61 (bp, 1H).

LC-MS (Method 1): Rₜ=3.092 min. MS (Mass method 1): m/z=425 (M+H)⁺

### Example 7

### rac-N-{[2,5-dioxo-4-(tetrahydro-2H-pyran-3-yl)imidazolidin-4-yl]methyl}-2-phenyl-2H-1,2,3-triazole-4-carboxamide

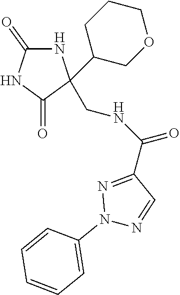 [SMILES: O=C1NC(=O)C(CNC(=O)c2cnn(-c3ccccc3)n2)(C2CCCOC2)N1]

The compound was synthesized according to the General Method A using 53 mg (0.28 mmol) of 2-phenyl-2H-1,2,3-triazole-4-carboxylic acid, 70 mg (0.28 mmol) of rac-5-(aminomethyl)-5-[oxan-3-yl]imidazolidine-2,4-dione-hydrogen chloride, 81 mg (0.42 mmol) of 1-(3-dimethylaminopropyl)-3-ethylcarbodiimide hydrochloride, 0.42 mL (0.42 mmol) of 1M 1-hydroxy-7-azabenzotriazole in N,N-dimethylacetamide and 0.12 mL (0.84 mmol) of triethylamine in 3 mL of N,N-dimethylformamide. The crude product obtained from the workup was purified by flash column chromatography on silica gel eluting with dichloromethane/methanol mixtures to afford 60 mg (55% yield, 99% purity) of the racemic product as a white powder.

¹H-NMR (300 MHz, DMSO-d₆): δ [ppm]=1.34-1.68 (m, 3H), 1.87-2.02 (m, 2H), 3.11-3.23 (m, 3H), 3.54-3.84 (m, 4H), 3.98-4.14 (m, 1H), 7.47-7.55 (m, 1H), 7.63 (t, 2H), 7.87 (s, 1H), 8.08 (d, 2H), 8.37-8.45 (m, 1H), 8.47 (s, 1H), 10.76 (bp, 1H).

LC-MS (Method 1): Rₜ=2.272 min. MS (Mass method 1): m/z=385 (M+H)⁺

### Example 8

### N-{[(4R)-4-cyclopropyl-2,5-dioxoimidazolidin-4-yl]methyl}-2-(3,4-difluorophenyl)-2H-1,2,3-triazole-4-carboxamide

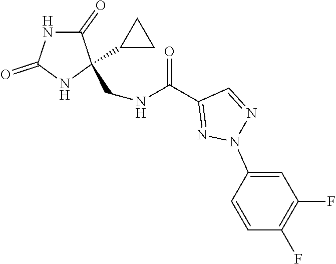 [SMILES: O=C1NC(=O)[C@](CNC(=O)c2cnn(-c3ccc(F)c(F)c3)n2)(C2CC2)N1]

The compound was synthesized according to the General Method A using 75 mg (0.33 mmol) of 2-(3,4-difluorophenyl)-2H-1,2,3-triazole-4-carboxylic acid, 75 mg (0.37 mmol) of (5R)-5-(aminomethyl)-5-cyclopropylimidazolidine-2,4-dione hydrochloride, 83 mg (0.43 mmol) of 1-(3-dimethylaminopropyl)-3-ethylcarbodiimide hydrochloride, 66 mg (0.43 mmol) of 1-hydroxybenzotriazole and 0.16 mL (0.93 mmol) of N,N-diisopropylethylamine in 3 mL of N,N-dimethylformamide. The crude product obtained from the workup was precipitated with diethyl ether and heptane to give 32 mg (23% yield, 99% purity) of the pure compound as an off-white powder.

¹H-NMR (300 MHz, DMSO-d₆): δ [ppm]=0.08-0.20 (m, 1H), 0.27-0.52 (m, 3H), 1.10-1.25 (m, 1H), 3.70 (ddd, 2H), 7.61 (s, 1H), 7.73 (q, 1H), 7.88-7.97 (m, 1H), 8.07-8.17 (m, 1H), 8.47-8.59 (m, 2H), 10.66 (bp, 1H).

LC-MS (Method 1): Rₜ=2.600 min. MS (Mass method 1): m/z=377 (M+H)⁺

### Example 9

### 2-(4-chlorophenyl)-N-{[(4R)-4-cyclopropyl-2,5-dioxoimidazolidin-4-yl]methyl}-2H-1,2,3-triazole-4-carboxamide

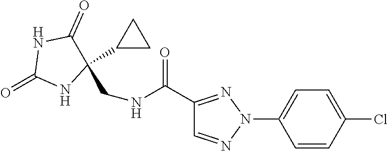 [SMILES: O=C1NC(=O)[C@](CNC(=O)c2cnn(-c3ccc(Cl)cc3)n2)(C2CC2)N1]

The compound was synthesized according to the General Method A using 82 mg (0.36 mmol) of 2-(4-chlorophenyl)-2H-1,2,3-triazole-4-carboxylic acid, 75 mg (0.36 mmol) of (5R)-5-(aminomethyl)-5-cyclopropylimidazolidine-2,4-dione hydrochloride, 105 mg (0.55 mmol) of 1-(3-dimethylaminopropyl)-3-ethylcarbodiimide hydrochloride, 0.55 mL (0.55 mmol) of 1M 1-hydroxy-7-azabenzotriazole in N,N-dimethylacetamide and 0.15 mL (1.10 mmol) of triethylamine in 3 mL of N,N-dimethylformamide. The crude product obtained from the workup was precipitated with diethyl ether and heptane to give 63 mg (50% yield, 99% purity) of the pure compound as an off-white powder.

¹H-NMR (300 MHz, DMSO-d₆): δ [ppm]=0.07-0.20 (m, 1H), 0.28-0.39 (m, 1H), 0.40-0.50 (m, 2H), 1.10-1.23 (m, 1H), 3.70 (ddd, 2H), 7.63 (s, 1H), 7.70 (d, 2H), 8.08 (d, 2H), 8.46-8.56 (m, 2H), 10.64 (bp, 1H).

LC-MS (Method 1): Rₜ=2.731 min. MS (Mass method 1): m/z=375 (M+H)⁺

### Example 10

### 2-(3-chlorophenyl)-N-{[(4R)-4-cyclopropyl-2,5-dioxoimidazolidin-4-yl]methyl}-2H-1,2,3-triazole-4-carboxamide

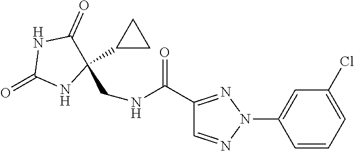 [SMILES: O=C1NC(=O)[C@](CNC(=O)c2cnn(-c3cccc(Cl)c3)n2)(C2CC2)N1]

The compound was synthesized according to the General Method A using 82 mg (0.36 mmol) of 2-(3-chlorophenyl)-2H-1,2,3-triazole-4-carboxylic acid, 75 mg (0.36 mmol) of (5R)-5-(aminomethyl)-5-cyclopropylimidazolidine-2,4-dione hydrochloride, 105 mg (0.55 mmol) of 1-(3-dimethylaminopropyl)-3-ethylcarbodiimide hydrochloride, 0.55 mL (0.55 mmol) of 1M 1-hydroxy-7-azabenzotriazole in N,N-dimethylacetamide and 0.15 mL (1.10 mmol) of triethylamine in 3 mL of N,N-dimethylformamide. The crude product obtained from the workup was precipitated with diethyl ether and heptane to give 85 mg (66% yield, 99% purity) of the pure compound as an off-white powder.

¹H-NMR (300 MHz, DMSO-d₆): δ [ppm]=0.07-0.20 (m, 1H), 0.28-0.50 (m, 3H), 1.11-1.23 (m, 1H), 3.70 (ddd, 2H), 7.54-7.70 (m, 3H), 8.04 (d, 1H), 8.11 (s, 1H), 8.50 (s, 1H), 8.57 (t, 1H), 10.66 (s, 1H

LC-MS (Method 1): Rₜ=2.735 min. MS (Mass method 1): m/z=375 (M+H)⁺

### Example 11

### 2-(2-chlorophenyl)-N-{[(4R)-4-cyclopropyl-2,5-dioxoimidazolidin-4-yl]methyl}-2H-1,2,3-triazole-4-carboxamide

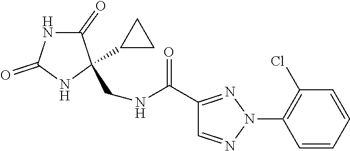 [SMILES: O=C1NC(=O)[C@](CNC(=O)c2cnn(-c3ccccc3Cl)n2)(C2CC2)N1]

The compound was synthesized according to the General Method A using 82 mg (0.36 mmol) of 2-(2-chlorophenyl)-2H-1,2,3-triazole-4-carboxylic acid, 75 mg (0.36 mmol) of (5R)-5-(aminomethyl)-5-cyclopropylimidazolidine-2,4-dione hydrochloride, 105 mg (0.55 mmol) of 1-(3-dimethylaminopropyl)-3-ethylcarbodiimide hydrochloride, 0.55 mL (0.55 mmol) of 1M 1-hydroxy-7-azabenzotriazole in N,N-dimethylacetamide and 0.15 mL (1.10 mmol) of triethylamine in 3 mL of N,N-dimethylformamide. The crude product obtained from the workup was precipitated with diethyl ether and heptane to give 74 mg (61% yield, 98% purity) of the pure compound as an off-white powder.

¹H-NMR (300 MHz, DMSO-d₆): δ [ppm]=0.06-0.17 (m, 1H), 0.28-0.50 (m, 3H), 1.10-1.20 (m, 1H), 3.69 (d, 2H), 7.55-7.71 (m, 3H), 7.79 (d, 2H), 8.44 (t, 1H), 8.50 (s, 1H), 10.64 (s, 1H).

LC-MS (Method 1): Rₜ=2.365 min. MS (Mass method 1): m/z=375 (M+H)⁺

### Example 12

### N-{[(4R)-4-cyclopropyl-2,5-dioxoimidazolidin-4-yl]methyl}-2-(2-methylphenyl)-2H-1,2,3-triazole-4-carboxamide

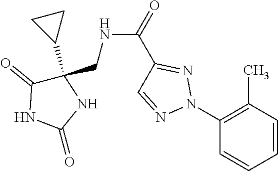 [SMILES: Cc1ccccc1-n1ncc(C(=O)NC[C@@]2(C3CC3)NC(=O)NC2=O)n1]

The compound was synthesized according to the General Method A using 74 mg (0.36 mmol) of 2-(2-methylphenyl)-2H-1,2,3-triazole-4-carboxylic acid, 75 mg (0.36 mmol) of (5R)-5-(aminomethyl)-5-cyclopropylimidazolidine-2,4-dione hydrochloride, 105 mg (0.55 mmol) of 1-(3-dimethylaminopropyl)-3-ethylcarbodiimide hydrochloride, 0.55 mL (0.55 mmol) of 1M 1-hydroxy-7-azabenzotriazole in N,N-dimethylacetamide and 0.15 mL (1.10 mmol) of triethylamine in 3 mL of N,N-dimethylformamide. The crude product obtained from the workup was precipitated with diethyl ether and heptane to give 76 mg (58% yield, 99% purity) of the pure compound as an off-white powder.

¹H-NMR (300 MHz, DMSO-d₆): δ [ppm]=0.06-0.18 (m, 1H), 0.26-0.50 (m, 3H), 1.10-1.21 (m, 1H), 2.28 (s, 3H), 3.69 (d, 2H), 7.37-7.52 (m, 3H), 7.57-7.64 (m, 2H), 8.38 (t, 1H), 8.44 (s, 1H), 10.65 (s, 1H).

LC-MS (Method 1): Rₜ=2.443 min. MS (Mass method 1): m/z=355 (M+H)⁺

### Example 13

### N-{[(4R)-4-cyclopropyl-2,5-dioxoimidazolidin-4-yl]methyl}-2-(4-methylphenyl)-2H-1,2,3-triazole-4-carboxamide

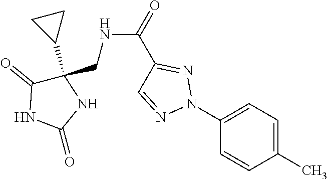 [SMILES: Cc1ccc(-n2ncc(C(=O)NC[C@@]3(C4CC4)NC(=O)NC3=O)n2)cc1]

The compound was synthesized according to the General Method A using 74 mg (0.36 mmol) of 2-(4-methylphenyl)-2H-1,2,3-triazole-4-carboxylic acid, 75 mg (0.36 mmol) of (5R)-5-(aminomethyl)-5-cyclopropylimidazolidine-2,4-dione hydrochloride, 105 mg (0.55 mmol) of 1-(3-dimethylaminopropyl)-3-ethylcarbodiimide hydrochloride, 0.55 mL (0.55 mmol) of 1M 1-hydroxy-7-azabenzotriazole in N,N-dimethylacetamide and 0.15 mL (1.10 mmol) of triethylamine in 3 mL of N,N-dimethylformamide. The crude product obtained from the workup was chromatographed in dichloromethane/methanol mixtures to give 73 mg (56% yield, 99% purity) of the pure compound as an off-white powder.

¹H-NMR (300 MHz, DMSO-d₆): δ [ppm]=0.07-0.20 (m, 1H), 0.29-0.51 (m, 3H), 1.11-1.26 (m, 1H), 2.39 (s, 3H), 3.70 (ddd, 2H), 7.42- (d, 2H), 7.62 (s, 1H), 7.95 (d, 2H), 8.39-8.49 (m, 2H), 10.66 (s, 1H).

LC-MS (Method 1): Rₜ=2.642 min. MS (Mass method 1): m/z=355 (M+H)⁺

### Example 14

### 2-(4-cyanophenyl)-N-{[(4R)-4-cyclopropyl-2,5-dioxoimidazolidin-4-yl]methyl}-2H-1,2,3-triazole-4-carboxamide

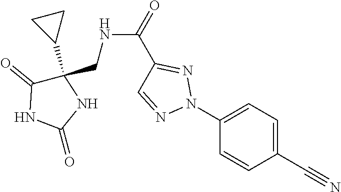 [SMILES: N#Cc1ccc(-n2ncc(C(=O)NC[C@@]3(C4CC4)NC(=O)NC3=O)n2)cc1]

The compound was synthesized according to the General Method A using 78 mg (0.36 mmol) of 2-(4-cyanophenyl)-2H-1,2,3-triazole-4-carboxylic acid, 75 mg (0.36 mmol) of (5R)-5-(aminomethyl)-5-cyclopropylimidazolidine-2,4-dione hydrochloride, 105 mg (0.55 mmol) of 1-(3-dimethylaminopropyl)-3-ethylcarbodiimide hydrochloride, 0.55 mL (0.55 mmol) of 1M 1-hydroxy-7-azabenzotriazole in N,N-dimethylacetamide and 0.15 mL (1.10 mmol) of triethylamine in 3 mL of N,N-dimethylformamide. The crude product obtained from the workup was chromatographed in dichloromethane/methanol mixtures to give 103 mg (77% yield, 99% purity) of the pure compound as an off-white powder.

¹H-NMR (300 MHz, DMSO-d₆): δ [ppm]=0.08-0.20 (m, 1H), 0.28-0.51 (m, 3H), 1.10-1.24 (m, 1H), 3.70 (ddd, 2H), 7.63 (s, 1H), 8.12 (d, 2H), 8.24 (d, 2H), 8.54-8.65 (m, 2H), 10.66 (s, 1H).

LC-MS (Method 1): Rₜ=2.311 min. MS (Mass method 1): m/z=366 (M+H)⁺

### Example 15

### N-{[(4R)-4-cyclopropyl-2,5-dioxoimidazolidin-4-yl]methyl}-2-(5-fluoropyridin-2-yl)-2H-1,2,3-triazole-4-carboxamide

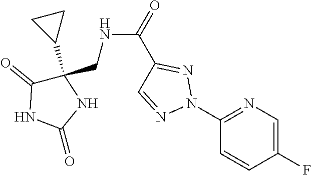 [SMILES: O=C1NC(=O)[C@](CNC(=O)c2cnn(-c3ccc(F)cn3)n2)(C2CC2)N1]

The compound was synthesized according to the General Method A using 111 mg (0.53 mmol) of 2-(5-fluoropyridin-2-yl)-2H-1,2,3-triazole-4-carboxylic acid, 100 mg (0.48 mmol) of (5R)-5-(aminomethyl)-5-cyclopropylimidazolidine-2,4-dione hydrochloride, 121 mg (0.63 mmol) of 1-(3-dimethylaminopropyl)-3-ethylcarbodiimide hydrochloride, 97 mg (0.63 mmol) of 1-hydroxybenzotriazole and 0.24 mL (1.40 mmol) of N,N-diisopropylethylamine in 3 mL of N,N-dimethylformamide. The crude product obtained from the workup was purified by reversed-phase chromatography using a gradient from 95% 65 mM ammonium acetate:acetonitrile 90:10/5% acetonitrile:methanol 1:1 to 63% 65 mM ammonium acetate:acetonitrile 90:10/37% acetonitrile:methanol 1:1 to give 5 mg (3% yield, 99% purity) of the pure compound as an off-white powder.

¹H-NMR (300 MHz, DMSO-d₆): δ [ppm]=0.07-0.20 (m, 1H), 0.29-0.49 (m, 3H), 1.10-1.22 (m, 1H), 3.71 (d, 2H), 7.61 (s, 1H), 7.99-8.19 (m, 2H), 8.48-8.59 (m, 2H), 8.65 (s, 1H), 10.65 (s, 1H).

LC-MS (Method 1): Rₜ=1.908 min. MS (Mass method 1): m/z=360 (M+H)⁺

### Example 16

### N-{[(4R)-4-cyclopropyl-2,5-dioxoimidazolidin-4-yl]methyl}-2-(pyrazin-2-yl)-2H-1,2,3-triazole-4-carboxamide

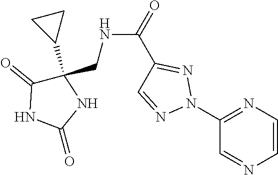 [SMILES: O=C1NC(=O)[C@](CNC(=O)c2cnn(-c3cnccn3)n2)(C2CC2)N1]

The compound was synthesized according to the General Method A using 60 mg (0.31 mmol) of 2-(pyrazin-2-yl)-2H-1,2,3-triazole-4-carboxylic acid, 71 mg (0.34 mmol) of (5R)-5-(aminomethyl)-5-cyclopropylimidazolidine-2,4-dione hydrochloride, 78 mg (0.41 mmol) of 1-(3-dimethylaminopropyl)-3-ethylcarbodiimide hydrochloride, 62 mg (0.41 mmol) of 1-hydroxybenzotriazole and 0.15 mL (0.88 mmol) of N,N-diisopropylethylamine in 3 mL of N,N-dimethylformamide. The crude product obtained from the workup was precipitated with diethyl ether and heptane to give 11 mg (10% yield, 99% purity) of the pure compound as an off-white powder.

¹H-NMR (300 MHz, DMSO-d₆): δ [ppm]=0.07-0.19 (m, 1H), 0.24-0.46 (m, 3H), 1.18-1.21 (m, 1H), 3.68 (ddd, 2H), 7.39 (bp, 1H), 8.57-8.66 (m, 2H), 8.72 (s, 1H), 8.84 (s, 1H), 9.38 (s, 1H), 10.65 (bp, 1H).

LC-MS (Method 1): Rₜ=1.580 min. MS (Mass method 1): m/z=343 (M+H)⁺

### Example 17

### 2-(3-cyanophenyl)-N-{[(4R)-4-cyclopropyl-2,5-dioxoimidazolidin-4-yl]methyl}-2H-1,2,3-triazole-4-carboxamide

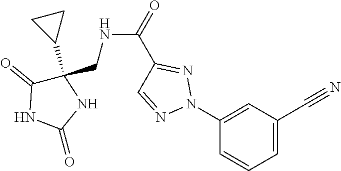 [SMILES: N#Cc1cccc(-n2ncc(C(=O)NC[C@@]3(C4CC4)NC(=O)NC3=O)n2)c1]

The compound was synthesized according to the General Method A using 78 mg (0.36 mmol) of 2-(3-cyanophenyl)-2H-1,2,3-triazole-4-carboxylic acid, 75 mg (0.36 mmol) of (5R)-5-(aminomethyl)-5-cyclopropylimidazolidine-2,4-dione hydrochloride, 105 mg (0.54 mmol) of 1-(3-dimethylaminopropyl)-3-ethylcarbodiimide hydrochloride, 0.54 mL (0.54 mmol) of 1M 1-hydroxy-7-azabenzotriazole in N,N-dimethylacetamide and 0.15 mL (1.10 mmol) of triethylamine in 2 mL of N,N-dimethylformamide. The crude product obtained from the workup was purified by flash column chromatography eluting with dichloromethane/methanol mixtures to give 49 mg (37% yield, 99% purity) of the enatiopure compound form as a white powder.

¹H-NMR (300 MHz, DMSO-d₆): δ [ppm]=0.08-0.19 (m, 1H), 0.28-0.39 (m, 1H), 0.40-0.52 (m, 2H), 1.11-1.24 (m, 1H), 3.71 (ddd, 2H), 7.63 (s, 1H), 7.84 (t, 1H), 7.98 (d, 1H), 8.38 (s, 1H), 8.48 (s, 1H), 8.54 (s, 1H), 8.59 (t, 1H), 10.66 (s, 1H).

LC-MS (Method 1): Rₜ=2.322 min. MS (Mass method 1): m/z=366 (M+H)⁺

### Example 18

### N-{[(4R)-4-cyclopropyl-2,5-dioxoimidazolidin-4-yl]methyl}-2-(3-methylphenyl)-2H-1,2,3-triazole-4-carboxamide

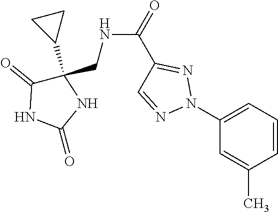 [SMILES: Cc1cccc(-n2ncc(C(=O)NC[C@@]3(C4CC4)NC(=O)NC3=O)n2)c1]

The compound was synthesized according to the General Method A using 74 mg (0.36 mmol) of 2-(3-methylphenyl)-2H-1,2,3-triazole-4-carboxylic acid, 75 mg (0.36 mmol) of (5R)-5-(aminomethyl)-5-cyclopropylimidazolidine-2,4-dione hydrochloride, 105 mg (0.54 mmol) of 1-(3-dimethylaminopropyl)-3-ethylcarbodiimide hydrochloride, 0.54 mL (0.54 mmol) of 1M 1-hydroxy-7-azabenzotriazole in N,N-dimethylacetamide and 0.15 mL (1.10 mmol) of triethylamine in 2 mL of N,N-dimethylformamide. The crude product obtained from the workup was purified by flash column chromatography eluting with dichloromethane/methanol mixtures to give 55 mg (42% yield, 99% purity) of the enatiopure compound form as a white powder.

¹H-NMR (300 MHz, DMSO-d₆): δ [ppm]=0.07-0.19 (m, 1H), 0.28-0.49 (m, 3H), 1.11-1.25 (m, 1H), 2.43 (s, 3H), 3.70 (ddd, 2H), 7.32 (d, 1H), 7.49 (t, 1H), 7.62 (s, 1H), 7.86 (d, 1H), 7.90 (s, 1H), 8.42-8.51 (m, 2H), 10.65 (s, 1H).

LC-MS (Method 1): Rₜ=2.728 min. MS (Mass method 1): m/z=355 (M+H)⁺

### Example 19

### rac-N-{[2,5-dioxo-4-(tetrahydrofuran-3-yl)imidazolidin-4-yl]methyl}-2-phenyl-2H-1,2,3-triazole-4-carboxamide

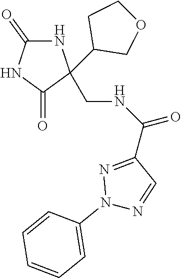 [SMILES: O=C1NC(=O)C(CNC(=O)c2cnn(-c3ccccc3)n2)(C2CCOC2)N1]

The compound was synthesized according to the General Method A using 60 mg (0.32 mmol) of 2-phenyl-2H-1,2,3-triazole-4-carboxylic acid, 75 mg (0.32 mmol) of rac-5-(aminomethyl)-5-[oxolan-3-yl]imidazolidine-2,4-dione-hydrogen chloride, 92 mg (0.48 mmol) of 1-(3-dimethylaminopropyl)-3-ethylcarbodiimide hydrochloride, 0.48 mL (0.48 mmol) of 1M 1-hydroxy-7-azabenzotriazole in N,N-dimethylacetamide and 0.13 mL (0.95 mmol) of triethylamine in 2 mL of N,N-dimethylformamide. The crude product obtained from the workup was purified by flash column chromatography eluting with dichloromethane/methanol mixtures to give 70 mg (54% yield, 99% purity) of the compound form as a white powder.

¹H-NMR (300 MHz, DMSO-d₆): δ [ppm]=1.52-1.87 (m, 1H), 1.96-2.10 (m, 1H), 2.56-2.71 (m, 1H), 3.47-3.80 (m, 5H), 7.51 (t, 1H), 7.63 (t, sH), 7.99 (d, 1H), 8.08 (d, 2H), 8.45-8.56 (m, 2H), 10.79 (bp, 1H).

LC-MS (Method 1): Rₜ=2.164 min. MS (Mass method 1): m/z=371 (M+H)⁺

### Example 20

### rac-N-{[2,5-dioxo-4-(pyridin-2-yl)imidazolidin-4-yl]methyl}-2-phenyl-2H-1,2,3-triazole-4-carboxamide

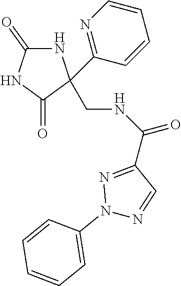 [SMILES: O=C1NC(=O)C(CNC(=O)c2cnn(-c3ccccc3)n2)(c2ccccn2)N1]

The compound was synthesized according to the General Method A using 195 mg (1.03 mmol) of 2-phenyl-2H-1,2,3-triazole-4-carboxylic acid, 250 mg (1.03 mmol) of rac-5-(aminomethyl)-5-(pyridin-2-yl)imidazolidine-2,4-dione-hydrogen chloride, 296 mg (1.55 mmol) of 1-(3-dimethylaminopropyl)-3-ethylcarbodiimide hydrochloride, 1.55 mL (1.55 mmol) of 1M 1-hydroxy-7-azabenzotriazole in N,N-dimethylacetamide and 0.43 mL (3.10 mmol) of triethylamine in 2 mL of N,N-dimethylformamide. The crude product obtained from the workup was purified by flash column chromatography eluting with dichloromethane/methanol mixtures to give 33 mg (8% yield, 97% purity) of the compound form as a white powder.

¹H-NMR (300 MHz, DMSO-d₆): δ [ppm]=4.18 (ddd, 2H), 7.41 (t, 1H), 7.46-7.66 (m, 4H), 7.87 (t, 1H), 8.05 (d, 2H), 8.40 (s, 1H), 8.45-8.52 (m, 2H), 8.64 (d, 1H), 10.92 (bp, 1H).

LC-MS (Method 1): Rₜ=2.413 min. MS (Mass method 1): m/z=378 (M+H)⁺

### Example 21

### rac-N-{[2,5-dioxo-4-(pyridin-4-yl)imidazolidin-4-yl]methyl}-2-phenyl-2H-1,2,3-triazole-4-carboxamide

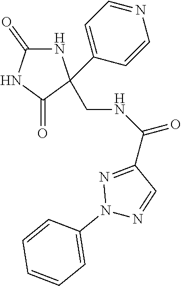 [SMILES: O=C1NC(=O)C(CNC(=O)c2cnn(-c3ccccc3)n2)(c2ccncc2)N1]

The compound was synthesized according to the General Method A using 185 mg (0.98 mmol) of 2-phenyl-2H-1,2,3-triazole-4-carboxylic acid, 238 mg (0.98 mmol) of rac-5-(aminomethyl)-5-(pyridin-4-yl)imidazolidine-2,4-dione hydrochloride, 282 mg (1.47 mmol) of 1-(3-dimethylaminopropyl)-3-ethylcarbodiimide hydrochloride, 1.47 mL (1.47 mmol) of 1M 1-hydroxy-7-azabenzotriazole in N,N-dimethylacetamide and 0.41 mL (2.90 mmol) of triethylamine in 2 mL of N,N-dimethylformamide. The crude product obtained from the workup was purified by flash column chromatography eluting with dichloromethane/methanol mixtures to give 3 mg (1% yield, 95% purity) of the compound form as a white powder.

¹H-NMR (300 MHz, DMSO-d₆): δ [ppm]=3.90-4.05 (m, 2H), 7.46-7.54 (m, 1H), 7.56-7.66 (m, 4H), 8.05 (d, 2H), 8.45 (s, 1H), 8.61 (d, 2H), 8.64-8.73 (m, 2H), 11.03 (bp, 1H).

LC-MS (Method 1): Rₜ=1.820 min. MS (Mass method 1): m/z=378 (M+H)⁺

### Example 22

### rac-N-{[2,5-dioxo-4-(pyridin-2-yl)imidazolidin-4-yl]methyl}-2-(4-fluorophenyl)-2H-1,2,3-triazole-4-carboxamide

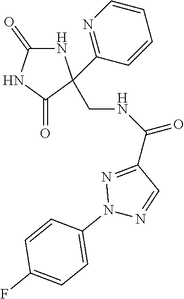 [SMILES: O=C1NC(=O)C(CNC(=O)c2cnn(-c3ccc(F)cc3)n2)(c2ccccn2)N1]

The compound was synthesized according to the General Method A using 179 mg (0.86 mmol) of 2-(4-fluorophenyl)-2H-1,2,3-triazole-4-carboxylic acid, 210 mg (0.86 mmol) of rac-5-(aminomethyl)-5-(pyridin-2-yl)imidazolidine-2,4-dione-hydrogen chloride, 249 mg (1.30 mmol) of 1-(3-dimethylaminopropyl)-3-ethylcarbodiimide hydrochloride, 1.30 mL (1.30 mmol) of 1M 1-hydroxy-7-azabenzotriazole in N,N-dimethylacetamide and 0.36 mL (2.60 mmol) of triethylamine in 2 mL of N,N-dimethylformamide. The crude product obtained from the workup was purified by flash column chromatography eluting with dichloromethane/methanol mixtures to give 32 mg (9% yield, 99% purity) of the compound form as a white powder.

¹H-NMR (300 MHz, DMSO-d₆): δ [ppm]=4.17 (ddd, 2H), 7.37-7.52 (m, 3H), 7.55 (d, 1H), 7.87 (t, 1H), 8.04-8.13 (m, 2H), 8.40 (s, 1H), 8.44-8.54 (m, 2H), 8.64 (d, 1H), 10.90 (s, 1H).

LC-MS (Method 1): Rₜ=2.514 min. MS (Mass method 1): m/z=396 (M+H)⁺

### Example 23

### rac-N-{[2,5-dioxo-4-(pyridin-3-yl)imidazolidin-4-yl]methyl}-2-(4-fluorophenyl)-2H-1,2,3-triazole-4-carboxamide

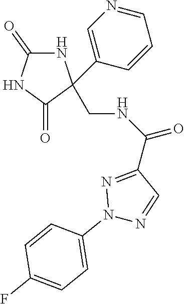 [SMILES: O=C1NC(=O)C(CNC(=O)c2cnn(-c3ccc(F)cc3)n2)(c2cccnc2)N1]

The compound was synthesized according to the General Method A using 213 mg (1.03 mmol) of 2-(4-fluorophenyl)-2H-1,2,3-triazole-4-carboxylic acid, 250 mg (1.03 mmol) of rac-5-(aminomethyl)-5-(pyridin-3-yl)imidazolidine-2,4-dione-hydrogen chloride, 296 mg (1.50 mmol) of 1-(3-dimethylaminopropyl)-3-ethylcarbodiimide hydrochloride, 1.50 mL (1.50 mmol) of 1M 1-hydroxy-7-azabenzotriazole in N,N-dimethylacetamide and 0.43 mL (3.10 mmol) of triethylamine in 2 mL of N,N-dimethylformamide. The crude product obtained from the workup was purified by flash column chromatography eluting with dichloromethane/methanol mixtures to give 6 mg (1% yield, 98% purity) of the compound form as a white powder.

¹H-NMR (300 MHz, DMSO-d₆): δ [ppm]=3.89-4.06 (m, 2H), 7.41-7.53 (m, 3H), 7.99 (d, 1H), 8.03-8.12 (m, 2H), 8.44 (s, 1H), 8.55 (d, 1H), 8.65-8.73 (m, 2H), 8.79 (s, 1H), 11.03 (bp, 1H).

LC-MS (Method 1): Rₜ=2.031 min. MS (Mass method 1): m/z=396 (M+H)⁺

### Example 24

### rac-N-{[2,5-dioxo-4-(pyridin-4-yl)imidazolidin-4-yl]methyl}-2-(4-fluorophenyl)-2H-1,2,3-triazole-4-carboxamide

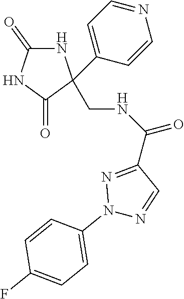 [SMILES: O=C1NC(=O)C(CNC(=O)c2cnn(-c3ccc(F)cc3)n2)(c2ccncc2)N1]

The compound was synthesized according to the General Method A using 125 mg (0.61 mmol) of 2-(4-fluorophenyl)-2H-1,2,3-triazole-4-carboxylic acid, 147 mg (0.61 mmol) of rac-5-(aminomethyl)-5-(pyridin-4-yl)imidazolidine-2,4-dione-hydrogen chloride, 174 mg (0.91 mmol) of 1-(3-dimethylaminopropyl)-3-ethylcarbodiimide hydrochloride, 0.91 mL (0.91 mmol) of 1M 1-hydroxy-7-azabenzotriazole in N,N-dimethylacetamide and 0.25 mL (1.80 mmol) of triethylamine in 2 mL of N,N-dimethylformamide. The crude product obtained from the workup was purified by flash column chromatography eluting with dichloromethane/methanol mixtures to give 27 mg (11% yield, 99% purity) of the compound form as a white powder.

¹H-NMR (300 MHz, DMSO-d₆): δ [ppm]=3.94-4.01 (m, 2H), 7.47 (t, 2H), 7.63 (d, 2H), 8.04-8.11 (m, 2H), 8.44 (s, 1H), 8.61-8.73 (m, 4H), 11.05 (s, 1H).

LC-MS (Method 1): Rₜ=1.936 min. MS (Mass method 1): m/z=396 (M+H)⁺

### Example 25

### N-{[(4R)-4-cyclopropyl-2,5-dioxoimidazolidin-4-yl]methyl}-3-[1-(cyclopropylmethyl)-1H-pyrazol-4-yl]-1,2-oxazole-5-carboxamide

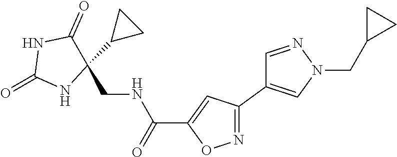 [SMILES: O=C1NC(=O)[C@](CNC(=O)c2cc(-c3cnn(CC4CC4)c3)no2)(C2CC2)N1]

929 mg (2.44 mmol) of HATU were added to a solution of 380 mg (1.63 mmol) of 3-[1-(cyclopropylmethyl)-1H-pyrazol-4-yl]-1,2-oxazole-5-carboxylic acid, 335 mg (1.63 mmol) of (5R)-5-(aminomethyl)-5-cyclopropylimidazolidine-2,4-dione hydrochloride, 2.40 mL (2.40 mmol) of 1M 1-hydroxy-7-azabenzotriazole in N,N-dimethylacetamide and 0.85 mL (4.90 mmol) of N,N-diisopropylethylamine in 5 mL of N,N-dimethylformamide and the reaction was allowed to stir at room temperature for 16 hours. The solvent was evaporated and the residue was diluted with dichloromethane, sequentially washed with an aqueous solution of sodium hydrogencarbonate(aq.) and 2N hydrochloric acid(aq.). The organic layer was dried over magnesium sulfate, filtered and concentrated to afford a residue, which was further purified by flash chromatography on silica gel eluting with dichloromethane/methanol mixtures to give 20 mg (3% yield, 99% purity) of the product as a white solid.

¹H-NMR (300 MHz, DMSO-d₆): δ [ppm]=0.07-0.19 (m, 1H), 0.28-0.50 (m, 5H), 0.51-0.67 (m, 2H), 1.09-1.19 (m, 1H), 1.23-1.34 (m, 1H), 3.66 (ddd, 2H), 4.03 (d, 2H), 7.39 (s, 1H), 7.94 (s, 1H), 8.38 (s, 1H), 8.87 (t, 1H), 10.64 (s, 1H).

LC-MS (Method 1): Rₜ=2.166 min. MS (Mass method 1): m/z=385 (M+H)⁺

### Example 26

### rac-{[4-tert-butyl-2,5-dioxoimidazolidin-4-yl]methyl}-2-phenyl-1,3-oxazole-5-carboxamide

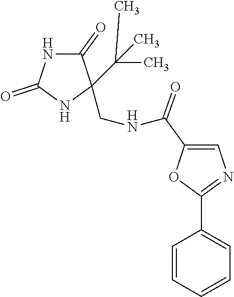 [SMILES: CC(C)(C)C1(CNC(=O)c2cnc(-c3ccccc3)o2)NC(=O)NC1=O]

226 mg (0.59 mmol) of HATU were added to a solution of 75 mg (0.39 mmol) of 2-phenyl-1,3-oxazole-5-carboxylic acid, 73 mg (0.39 mmol) of rac-5-(aminomethyl)-5-tert-butylimidazolidine-2,4-dione, 0.59 mL (0.59 mmol) of 1M 1-hydroxy-7-azabenzotriazole in N,N-dimethylacetamide and 0.17 mL, (1.20 mmol) of triethylamine in 5 mL of N,N-dimethylformamide and the reaction was allowed to stir at room temperature for 1 hour. The reaction was quenched with methanol and the solvent was removed in vacuo. The corresponding residue was diluted with ethyl acetate and sequentially washed with 1N hydrochloric acid(aq.), saturated sodium hydrogencarbonate(aq.) and brine. The organic layer was dried over magnesium sulfate, filtered, concentrated and purified by flash chromatography on silica gel eluting with dichloromethane and methanol to give 86 mg (58% yield, 95% purity) of the racemic product as a white powder.

¹H-NMR (300 MHz, DMSO-d₆): δ [ppm]=1.03 (s, 9H), 3.71 (ddd, 2H), 7.53-7.65 (m, 4H), 7.90 (s, 1H), 8.06-8.15 (m, 2H), 8.61 (t, 1H), 10.61 (bp, 1H).

LC-MS (Method 1): Rₜ=2.385 min. MS (Mass method 1): m/z=357 (M+H)⁺

### Example 27

### N-{[(4R)-4-cyclopropyl-2,5-dioxoimidazolidin-4-yl]methyl}-2-(4-methoxyphenyl)-2H-1,2,3-triazole-4-carboxamide

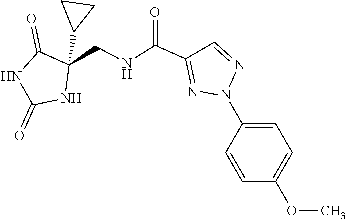 [SMILES: COc1ccc(-n2ncc(C(=O)NC[C@@]3(C4CC4)NC(=O)NC3=O)n2)cc1]

2-(4-Methoxyphenyl)-2H-1,2,3-triazole-4-carboxylic acid (53.3 mg, 243 μmol) dissolved in 3 ml DMF were treated with (5R)-5-(aminomethyl)-5-cyclopropylimidazolidine-2,4-dione hydrochloride (50.0 mg, 243 μmol), 1-ethyl-3-(3-dimethylaminopropyl)carbodiimide hydrochloride (60.6 mg, 316 μmol), 1-hydroxybenzotriazole hydrate (48.4 mg, 316 μmol) and N,N-diisopropylethylamine (120 μl, 681 μmol). The mixture was stirred over night at room temperature. Purification was done by preparative HPLC (column: Chromatorex C1810 μm, 250×30 mm; eluent A=water, B=acetonitrile; gradient: 0.0 min 5% B; 3 min 5% B; 20 min 50% B; 23 min 100% B; 26 min 5% B; flow: 50 ml/min; 0.1% formic acid). Product containing samples were united, the solvents were evaporated and the residue was lyophylized. 28.0 mg (100% purity, 31% yield) of the title compound were obtained.

LC-MS (Method 7): Rₜ=1.29 min; MS (ESIpos): m/z=371 [M+H]⁺

¹H-NMR (400 MHz, DMSO-d6) δ [ppm]: 0.115 (0.52), 0.124 (0.71), 0.129 (0.69), 0.137 (0.93), 0.145 (0.78), 0.154 (0.66), 0.325 (0.69), 0.335 (0.74), 0.343 (0.98), 0.352 (0.72), 0.358 (0.59), 0.362 (0.58), 0.373 (0.44), 0.414 (1.29), 0.431 (2.47), 0.450 (2.11), 0.469 (0.75), 1.150 (0.73), 1.167 (1.08), 1.183 (0.73), 3.311 (16.00), 3.639 (0.63), 3.654 (0.79), 3.673 (1.68), 3.688 (1.52), 3.709 (1.53), 3.725 (1.64), 3.743 (0.70), 3.759 (0.65), 7.150 (4.70), 7.168 (1.69), 7.173 (4.94), 7.181 (0.56), 7.626 (3.67), 7.957 (0.54), 7.966 (4.94), 7.971 (1.71), 7.983 (1.62), 7.988 (4.67), 7.997 (0.51), 8.396 (0.93), 8.406 (6.71), 8.428 (0.86), 10.653 (1.92).

### Example 28

### N-{[(4R)-4-cyclopropyl-2,5-dioxoimidazolidin-4-yl]methyl}-2-[4-(methylsulfonyl)phenyl]-2H-1,2,3-triazole-4-carboxamide

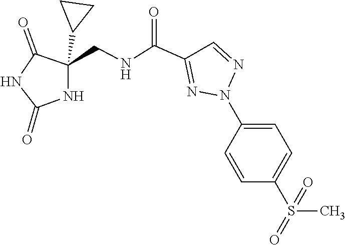 [SMILES: CS(=O)(=O)c1ccc(-n2ncc(C(=O)NC[C@@]3(C4CC4)NC(=O)NC3=O)n2)cc1]

The reaction was carried out analogously to the preparation of N-{[(4R)-4-cyclopropyl-2,5-dioxoimidazolidin-4-yl]methyl}-2-(4-methoxyphenyl)-2H-1,2,3-triazole-4-carboxamide. 74.0 mg (100% purity, 73% yield) of the title compound were obtained.

LC-MS (Method 7): Rₜ=1.04 min; MS (ESIpos): m/z=419 [M+H]⁺

¹H-NMR (400 MHz, DMSO-d6) δ [ppm]: 0.445 (0.64), 0.462 (0.53), 3.300 (6.16), 3.313 (16.00), 3.677 (0.46), 3.692 (0.40), 3.733 (0.41), 3.750 (0.44), 7.637 (0.98), 8.173 (1.28), 8.178 (0.50), 8.190 (0.56), 8.195 (1.83), 8.309 (1.77), 8.314 (0.58), 8.326 (0.47), 8.331 (1.29), 8.576 (2.12), 8.607 (0.50), 10.660 (0.71).

### Example 29

### 2-(3-chloro-4-fluorophenyl)-N-{[(4R)-4-cyclopropyl-2,5-dioxoimidazolidin-4-yl]methyl}-2H-1,2,3-triazole-4-carboxamide

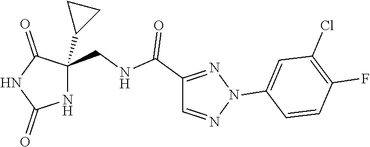 [SMILES: O=C1NC(=O)[C@](CNC(=O)c2cnn(-c3ccc(F)c(Cl)c3)n2)(C2CC2)N1]

The reaction was carried out analogously to the preparation of N-{[(4R)-4-cyclopropyl-2,5-dioxoimidazolidin-4-yl]methyl}-2-(4-methoxyphenyl)-2H-1,2,3-triazole-4-carboxamide. 53.0 mg (100% purity, 55% yield) of the title compound were obtained.

LC-MS (Method 7): Rₜ=1.49 min; MS (ESIpos): m/z=393 [M+H]⁺

¹H-NMR (400 MHz, DMSO-d₆) δ[ppm]: 0.108 (0.53), 0.119 (1.07), 0.131 (1.73), 0.142 (1.93), 0.146 (1.75), 0.155 (1.53), 0.170 (0.74), 0.307 (0.42), 0.328 (1.32), 0.337 (1.39), 0.344 (1.90), 0.355 (1.70), 0.364 (1.17), 0.375 (0.93), 0.403 (0.49), 0.414 (1.63), 0.420 (1.65), 0.434 (4.36), 0.451 (3.41), 0.470 (1.54), 1.134 (0.72), 1.148 (1.46), 1.155 (1.54), 1.161 (1.16), 1.168 (2.24), 1.182 (1.48), 1.187 (1.23), 1.202 (0.59), 3.619 (1.68), 3.633 (1.90), 3.653 (3.19), 3.668 (2.84), 3.726 (2.89), 3.743 (3.11), 3.760 (1.84), 3.778 (1.71), 7.599 (6.08), 7.683 (3.48), 7.705 (7.25), 7.728 (3.93), 8.051 (2.05), 8.058 (2.45), 8.061 (2.42), 8.068 (2.31), 8.073 (2.05), 8.080 (2.34), 8.084 (2.08), 8.091 (1.99), 8.246 (3.14), 8.252 (3.05), 8.262 (3.22), 8.268 (2.91), 8.506 (16.00), 8.542 (1.69), 8.559 (3.29), 8.575 (1.65), 10.655 (2.72).

### Example 30

### 2-(4-chloro-3-fluorophenyl)-N-{[(4R)-4-cyclopropyl-2,5-dioxoimidazolidin-4-yl]methyl}-2H-1,2,3-triazole-4-carboxamide

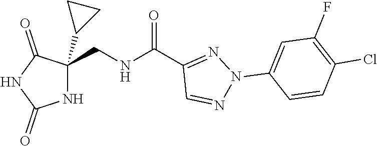 [SMILES: O=C1NC(=O)[C@](CNC(=O)c2cnn(-c3ccc(Cl)c(F)c3)n2)(C2CC2)N1]

The reaction was carried out analogously to the preparation of N-{[(4R)-4-cyclopropyl-2,5-dioxoimidazolidin-4-yl]methyl}-2-(4-methoxyphenyl)-2H-1,2,3-triazole-4-carboxamide. 23.0 mg (100% purity, 24% yield) of the title compound were obtained.

LC-MS (Method 7): Rₜ=1.50 min; MS (ESIpos): m/z=393 [M+H]⁺

¹H-NMR (400 MHz, DMSO-d6) δ [ppm]: 0.008 (0.82), 0.108 (0.64), 0.120 (1.37), 0.131 (2.15), 0.142 (2.38), 0.146 (2.23), 0.156 (1.89), 0.170 (0.93), 0.310 (0.54), 0.331 (1.66), 0.340 (1.74), 0.347 (2.32), 0.358 (2.13), 0.367 (1.43), 0.378 (1.12), 0.407 (0.59), 0.419 (2.00), 0.425 (2.02), 0.438 (5.36), 0.455 (4.16), 0.475 (1.89), 1.136 (0.88), 1.150 (1.79), 1.156 (1.91), 1.163 (1.46), 1.170 (2.77), 1.183 (1.78), 1.190 (1.52), 1.203 (0.71), 2.328 (0.50), 2.366 (0.54), 2.523 (2.19), 2.670 (0.54), 2.710 (0.54), 3.621 (2.10), 3.636 (2.37), 3.655 (3.94), 3.670 (3.54), 3.729 (3.58), 3.746 (3.85), 3.763 (2.23), 3.780 (2.10), 7.628 (9.15), 7.857 (2.94), 7.879 (5.75), 7.898 (5.32), 7.934 (4.52), 7.940 (4.35), 7.956 (2.40), 7.962 (2.49), 8.074 (4.08), 8.081 (3.79), 8.100 (4.03), 8.106 (3.79), 8.529 (16.00), 8.549 (2.20), 8.565 (4.19), 8.580 (2.09), 10.654 (4.82).

### Example 31

### N-{[(4R)-4-cyclopropyl-2,5-dioxoimidazolidin-4-yl]methyl}-2-(2,4-difluorophenyl)-2H-1,2,3-triazole-4-carboxamide

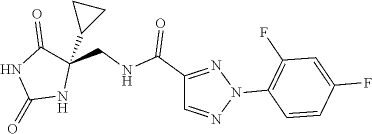 [SMILES: O=C1NC(=O)[C@](CNC(=O)c2cnn(-c3ccc(F)cc3F)n2)(C2CC2)N1]

2-(2,4-difluorophenyl)-2H-1,2,3-triazole-4-carboxylic acid (54.7 mg, 243 μmol) dissolved in 5 ml DCM were treated with (5R)-5-(aminomethyl)-5-cyclopropylimidazolidine-2,4-dione hydrochloride (50.0 mg, 243 μmol), 1-ethyl-3-(3-dimethylaminopropyl)carbodiimide hydrochloride (60.6 mg, 316 μmol), 1-hydroxybenzotriazole hydrate (48.4 mg, 316 μmol) and N,N-diisopropylethylamine (120 μl, 681 μmol). The mixture was stirred over night at room temperature. Then the mixture was diluted with ethyl acetate and washed with water and brine. The organic layer was dried over sodium sulfate, filtered and evaporated. The residue was put on Isolute and purified by chromatography on silica gel (10 g Snap Cartridge Biotage®; Biotage-Isolera-One®; DCM/MeOH-gradient: 2% MeOH-20% MeOH; flow: 36 ml/min). Samples containing the desired product were united, the solvents were evaporated and the residue was was lyophylized. 67.0 mg (100% purity, 73% yield) of the title compound were obtained.

LC-MS (Method 7): Rₜ=1.23 min; MS (ESIpos): m/z=377 [M+H]⁺

¹H-NMR (400 MHz, DMSO-d6) δ [ppm]: 0.008 (0.50), 0.098 (0.72), 0.109 (1.40), 0.117 (2.01), 0.131 (2.42), 0.140 (2.01), 0.147 (1.79), 0.159 (0.95), 0.299 (0.61), 0.310 (0.78), 0.318 (1.95), 0.330 (2.00), 0.337 (2.48), 0.346 (1.93), 0.356 (1.64), 0.367 (1.23), 0.404 (3.64), 0.421 (7.17), 0.440 (5.74), 0.458 (1.90), 1.122 (0.94), 1.139 (2.16), 1.156 (2.99), 1.172 (2.07), 1.189 (0.78), 3.644 (0.95), 3.659 (1.19), 3.678 (5.50), 3.691 (7.05), 3.706 (5.33), 3.724 (1.09), 3.740 (1.00), 7.351 (1.44), 7.354 (1.55), 7.357 (1.42), 7.374 (3.02), 7.377 (2.85), 7.393 (1.61), 7.396 (1.70), 7.400 (1.46), 7.587 (8.75), 7.657 (2.01), 7.664 (1.97), 7.680 (2.29), 7.685 (3.48), 7.691 (2.18), 7.707 (2.07), 7.714 (1.98), 7.930 (2.04), 7.945 (2.30), 7.952 (3.85), 7.967 (3.88), 7.974 (2.13), 7.989 (1.94), 8.443 (2.26), 8.459 (4.71), 8.474 (2.22), 8.517 (16.00), 10.646 (4.55).

### Example 32

### N-{[(4R)-4-cyclopropyl-2,5-dioxoimidazolidin-4-yl]methyl}-2-(3,4-dichlorophenyl)-2H-1,2,3-triazole-4-carboxamide

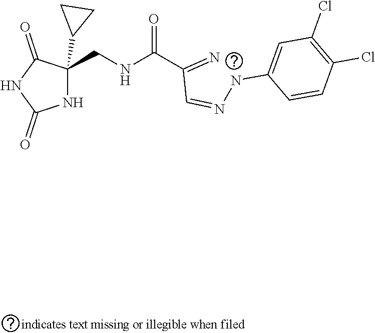 [SMILES: N=C(/C=N\Nc1ccc(Cl)c(Cl)c1)C(=O)NC[C@@]1(C2CC2)NC(=O)NC1=O]

The reaction was carried out analogously to the preparation of N-{[(4R)-4-cyclopropyl-2,5-dioxoimidazolidin-4-yl]methyl}-2-(4-methoxyphenyl)-2H-1,2,3-triazole-4-carboxamide. 54.0 mg (98% purity, 53% yield) of the title compound were obtained.

LC-MS (Method 7): Rₜ=1.62 min; MS (ESIpos): m/z=409 [M+H]⁺

¹H-NMR (400 MHz, DMSO-d₆) δ[ppm]: 0.108 (0.85), 0.121 (1.70), 0.132 (2.64), 0.142 (2.85), 0.146 (2.64), 0.156 (2.33), 0.170 (1.07), 0.309 (0.70), 0.330 (2.01), 0.339 (2.14), 0.346 (2.78), 0.357 (2.58), 0.366 (1.73), 0.377 (1.40), 0.405 (0.83), 0.416 (2.50), 0.422 (2.56), 0.436 (6.37), 0.453 (5.04), 0.472 (2.28), 1.136 (1.05), 1.150 (2.13), 1.157 (2.28), 1.163 (1.77), 1.171 (3.36), 1.184 (2.14), 1.190 (1.86), 1.204 (0.89), 1.234 (0.69), 1.370 (2.89), 3.619 (2.40), 3.634 (2.70), 3.653 (4.49), 3.668 (4.05), 3.729 (4.09), 3.746 (4.35), 3.763 (2.59), 3.780 (2.39), 7.610 (10.15), 7.901 (7.18), 7.923 (9.84), 8.040 (5.40), 8.046 (5.62), 8.062 (4.04), 8.068 (4.31), 8.291 (6.42), 8.297 (6.15), 8.528 (16.00), 8.575 (2.51), 8.591 (4.86), 8.606 (2.46), 10.654 (4.58).

### Example 33

### 2-(4-bromophenyl)-N-{[(4R)-4-cyclopropyl-2,5-dioxoimidazolidin-4-yl]methyl}-2H-1,2,3-triazole-4-carboxamide

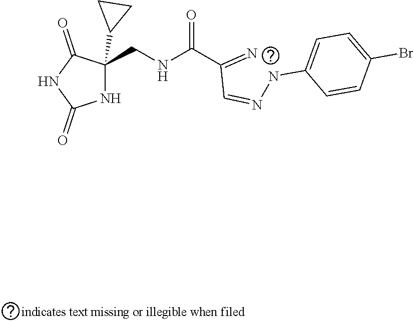 [SMILES: N=C(/C=N\Nc1ccc(Br)cc1)C(=O)NC[C@@]1(C2CC2)NC(=O)NC1=O]

The reaction was carried out analogously to the preparation of N-{[(4R)-4-cyclopropyl-2,5-dioxoimidazolidin-4-yl]methyl}-2-(4-methoxyphenyl)-2H-1,2,3-triazole-4-carboxamide. 15.0 mg (100% purity, 15% yield) of the title compound were obtained.

LC-MS (Method 7): Rₜ=1.52 min; MS (ESIpos): m/z=419 [M+H]⁺

¹H-NMR (400 MHz, DMSO-d6) δ [ppm]: −0.008 (1.30), 0.008 (1.10), 0.106 (0.54), 0.117 (1.08), 0.129 (1.48), 0.139 (2.00), 0.145 (1.61), 0.152 (1.37), 0.167 (0.71), 0.308 (0.47), 0.328 (1.34), 0.337 (1.64), 0.346 (2.07), 0.355 (1.54), 0.360 (1.21), 0.365 (1.17), 0.376 (0.94), 0.417 (2.40), 0.435 (4.65), 0.453 (4.16), 0.472 (1.51), 1.135 (0.71), 1.150 (1.37), 1.155 (1.47), 1.162 (1.23), 1.169 (2.20), 1.184 (1.44), 1.202 (0.58), 1.370 (0.71), 2.073 (1.58), 3.633 (1.50), 3.648 (1.71), 3.667 (3.37), 3.682 (2.98), 3.716 (3.02), 3.733 (3.29), 3.750 (1.64), 3.767 (1.51), 7.621 (7.17), 7.817 (1.16), 7.824 (10.02), 7.830 (3.41), 7.842 (4.09), 7.847 (12.95), 7.854 (1.65), 7.999 (1.57), 8.006 (12.76), 8.011 (3.79), 8.024 (3.57), 8.029 (10.04), 8.036 (1.21), 8.492 (16.00), 8.510 (3.54), 8.526 (1.73), 10.654 (5.38).

### Example 34

### N-{[(4R)-4-cyclopropyl-2,5-dioxoimidazolidin-4-yl]methyl}-2-(4-fluorophenyl)-2H-1,2,3-triazole-4-carboxamide

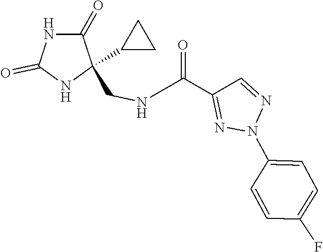 [SMILES: O=C1NC(=O)[C@](CNC(=O)c2cnn(-c3ccc(F)cc3)n2)(C2CC2)N1]

2-(4-fluorophenyl)-2H-1,2,3-triazole-4-carboxylic acid (1.375 g, 6.64 mmol, CAS 833-60-3) were treated with 140 ml dichloromethane, N-ethyl-N-isopropylpropan-2-amine (2.402 g, 18.58 mmol), 1-ethyl-3-(3-dimethylaminopropyl)carbodiimide hydrochloride (1.654 g, 8.63 mmol) and 1-hydroxybenzotriazole hydrate (1.321 g, 8.63 mmol). The mixture was stirred for 5 minutes before (5R)-5-(aminomethyl)-5-cyclopropylimidazolidine-2,4-dione hydrochloride (1.365 g, 6.64 mmol) was added. The mixture was stirred over night at room temperature. The solvent was removed on a rotary evaporator. The residue was taken up with ethyl acetate and washed with water and saturated aqueous sodium chloride solution. The organic layer was dried over sodium sulfate, filtered, Isolute® was added and the solvent was evaporated on a rotary evaporator. The crude product was purified by column chromatography (Machine: Biotage® Isolera One; Column: Biotage® SNAP Ultra 50 g; Gradient: Dichloromethane/Methanol, 2% Methanol→20% Methanol; Flow: 100 ml/min). Product containing samples were united and the solvents were removed on a rotary evaporator. The residue was stirred with acetonitrile, filtered and washed with acetonitrile. The product was dried over the weekend in a vacuum oven at 50° C. 1.5 g (63% yield, 100% purity) of the title compound were obtained.

LC-MS (Method 7): Rₜ=1.28 min; MS (ESIpos): m/z=359 [M+H]⁺

¹H-NMR (500 MHz, DMSO-d₆) δ[ppm]: 0.112 (0.57), 0.122 (1.12), 0.131 (1.58), 0.139 (1.87), 0.143 (1.56), 0.151 (1.35), 0.162 (0.68), 0.317 (0.48), 0.326 (0.69), 0.334 (1.33), 0.342 (1.47), 0.348 (1.83), 0.356 (1.58), 0.363 (1.04), 0.372 (0.79), 0.416 (0.43), 0.425 (1.76), 0.428 (1.81), 0.440 (4.41), 0.454 (3.82), 0.470 (1.58), 1.144 (0.73), 1.156 (1.39), 1.161 (1.49), 1.166 (1.14), 1.172 (2.17), 1.183 (1.41), 1.187 (1.20), 1.198 (0.57), 3.644 (1.64), 3.656 (1.83), 3.671 (3.19), 3.683 (2.84), 3.725 (2.90), 3.738 (3.12), 3.752 (1.77), 3.766 (1.63), 7.461 (0.58), 7.468 (5.01), 7.472 (1.82), 7.486 (8.50), 7.499 (1.84), 7.503 (5.25), 7.510 (0.54), 7.638 (7.28), 8.078 (0.62), 8.085 (5.21), 8.089 (2.27), 8.094 (5.48), 8.098 (3.28), 8.103 (5.37), 8.108 (2.07), 8.112 (5.00), 8.119 (0.51), 8.475 (16.00), 8.484 (1.95), 8.497 (3.48), 8.509 (1.74), 10.671 (5.48).

### Example 35

### N-{[(4R)-4-cyclopropyl-2,5-dioxoimidazolidin-4-yl]methyl}-2-(2,3-difluorophenyl)-2H-1,2,3-triazole-4-carboxamide

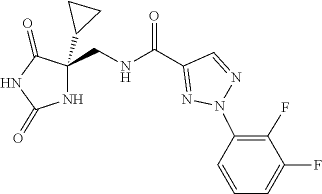 [SMILES: O=C1NC(=O)[C@](CNC(=O)c2cnn(-c3cccc(F)c3F)n2)(C2CC2)N1]

The reaction was carried out analogously to the preparation of N-{[(4R)-4-cyclopropyl-2,5-dioxoimidazolidin-4-yl]methyl}-2-(4-methoxyphenyl)-2H-1,2,3-triazole-4-carboxamide. 45.0 mg (100% purity, 49% yield) of the title compound were obtained.

LC-MS (Method 7): Rₜ=1.25 min; MS (ESIpos): m/z=377 [M+H]⁺

¹H-NMR (400 MHz, DMSO-d6) δ [ppm]: −0.008 (2.09), 0.008 (1.79), 0.103 (0.69), 0.114 (1.32), 0.123 (1.82), 0.129 (1.68), 0.136 (2.37), 0.142 (1.76), 0.147 (1.84), 0.152 (1.64), 0.165 (0.92), 0.306 (0.60), 0.316 (0.76), 0.325 (1.75), 0.336 (1.90), 0.344 (2.51), 0.353 (1.83), 0.359 (1.51), 0.363 (1.48), 0.373 (1.17), 0.413 (3.43), 0.430 (6.35), 0.449 (5.35), 0.468 (1.87), 1.130 (0.89), 1.147 (1.86), 1.158 (1.57), 1.164 (2.78), 1.180 (1.87), 1.198 (0.70), 1.371 (0.76), 2.525 (0.70), 3.653 (1.12), 3.668 (1.34), 3.687 (5.00), 3.703 (8.77), 3.719 (4.87), 3.737 (1.25), 3.754 (1.15), 7.446 (1.02), 7.451 (1.00), 7.459 (1.25), 7.467 (2.59), 7.472 (2.25), 7.480 (2.54), 7.486 (2.67), 7.493 (1.45), 7.502 (1.56), 7.506 (1.36), 7.621 (9.61), 7.653 (1.11), 7.657 (1.22), 7.675 (2.18), 7.678 (2.12), 7.682 (1.53), 7.692 (1.26), 7.696 (2.15), 7.700 (2.23), 7.718 (0.97), 7.722 (0.98), 7.754 (1.48), 7.758 (2.32), 7.762 (1.41), 7.770 (1.79), 7.774 (3.53), 7.779 (3.21), 7.791 (1.44), 7.795 (2.08), 7.799 (1.12), 8.480 (2.18), 8.495 (4.35), 8.511 (2.04), 8.567 (16.00), 10.653 (4.35).

### Example 36

### N-{[(4R)-4-cyclopropyl-2,5-dioxoimidazolidin-4-yl]methyl}-2-(2,5-difluorophenyl)-2H-1,2,3-triazole-4-carboxamide

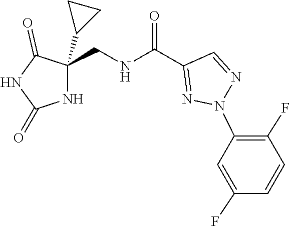 [SMILES: O=C1NC(=O)[C@](CNC(=O)c2cnn(-c3cc(F)ccc3F)n2)(C2CC2)N1]

The reaction was carried out analogously to the preparation of N-{[(4R)-4-cyclopropyl-2,5-dioxoimidazolidin-4-yl]methyl}-2-(4-methoxyphenyl)-2H-1,2,3-triazole-4-carboxamide. 45.0 mg (100% purity, 49% yield) of the title compound were obtained.

LC-MS (Method 7): Rₜ=1.22 min; MS (ESIpos): m/z=377 [M+H]⁺

¹H-NMR (400 MHz, DMSO-d6) δ [ppm]: −0.008 (1.72), 0.006 (1.18), 0.008 (1.47), 0.099 (0.65), 0.111 (1.25), 0.119 (1.78), 0.126 (1.60), 0.133 (2.19), 0.137 (1.65), 0.142 (1.73), 0.148 (1.60), 0.161 (0.87), 0.301 (0.57), 0.312 (0.72), 0.321 (1.72), 0.332 (1.79), 0.339 (2.25), 0.348 (1.71), 0.355 (1.44), 0.358 (1.45), 0.369 (1.12), 0.406 (3.24), 0.423 (6.24), 0.442 (5.09), 0.461 (1.71), 1.124 (0.83), 1.141 (1.87), 1.152 (1.50), 1.158 (2.61), 1.174 (1.82), 1.192 (0.67), 1.371 (1.36), 2.525 (0.52), 3.647 (0.89), 3.663 (1.06), 3.681 (4.83), 3.694 (6.34), 3.709 (4.75), 3.727 (1.02), 3.744 (0.91), 7.348 (1.19), 7.351 (1.39), 7.354 (1.52), 7.358 (1.35), 7.371 (2.52), 7.374 (2.76), 7.377 (2.59), 7.390 (1.36), 7.394 (1.48), 7.397 (1.59), 7.400 (1.39), 7.606 (9.10), 7.656 (1.84), 7.663 (1.80), 7.679 (2.09), 7.685 (3.09), 7.691 (2.02), 7.707 (1.88), 7.713 (1.82), 7.931 (1.88), 7.946 (2.10), 7.953 (3.49), 7.968 (3.53), 7.975 (1.96), 7.990 (1.79), 8.451 (1.98), 8.467 (4.19), 8.483 (1.98), 8.519 (16.00), 10.647 (3.19).

### Example 37

### N-{[(4R)-4-cyclopropyl-2,5-dioxoimidazolidin-4-yl]methyl}-2-(2-fluorophenyl)-2H-1,2,3-triazole-4-carboxamide

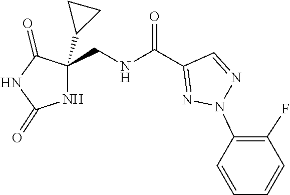 [SMILES: O=C1NC(=O)[C@](CNC(=O)c2cnn(-c3ccccc3F)n2)(C2CC2)N1]

The reaction was carried out analogously to the preparation of N-{[(4R)-4-cyclopropyl-2,5-dioxoimidazolidin-4-yl]methyl}-2-(4-methoxyphenyl)-2H-1,2,3-triazole-4-carboxamide. 25.0 mg (100% purity, 29% yield) of the title compound were obtained.

LC-MS (Method 7): Rₜ=1.16 min; MS (ESIpos): m/z=359 [M+H]⁺

¹H-NMR (400 MHz, DMSO-d6) δ [ppm]: 0.099 (0.68), 0.110 (1.31), 0.118 (1.87), 0.132 (2.21), 0.141 (1.92), 0.148 (1.64), 0.160 (0.92), 0.301 (0.60), 0.320 (1.84), 0.332 (1.85), 0.338 (2.26), 0.348 (1.77), 0.357 (1.53), 0.368 (1.16), 0.407 (3.35), 0.424 (6.66), 0.443 (5.36), 0.461 (1.77), 1.126 (0.84), 1.143 (1.99), 1.160 (2.74), 1.176 (1.92), 1.193 (0.72), 1.370 (2.45), 3.650 (0.89), 3.666 (1.07), 3.684 (5.17), 3.696 (6.15), 3.700 (6.04), 3.712 (5.05), 3.729 (1.03), 3.746 (0.90), 7.436 (1.99), 7.439 (2.09), 7.456 (4.50), 7.477 (2.79), 7.548 (1.56), 7.551 (1.70), 7.569 (3.26), 7.573 (3.21), 7.579 (1.80), 7.597 (3.65), 7.600 (3.55), 7.613 (10.12), 7.621 (2.98), 7.626 (3.10), 7.630 (2.41), 7.640 (1.87), 7.644 (2.08), 7.652 (1.00), 7.661 (0.79), 7.665 (0.78), 7.865 (2.34), 7.869 (2.32), 7.885 (4.33), 7.889 (4.12), 7.904 (2.21), 7.908 (2.05), 8.442 (1.97), 8.458 (4.10), 8.473 (2.01), 8.517 (16.00), 10.648 (3.42).

### Example 38

### ent-N-{[2,5-dioxo-4-(pyridin-2-yl)imidazolid in-4-yl]methyl}-2-phenyl-2H-1,2,3-triazole-4-carboxamide

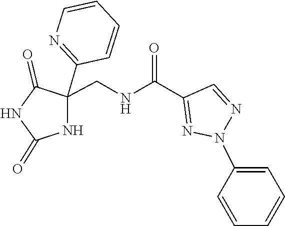 [SMILES: O=C1NC(=O)C(CNC(=O)c2cnn(-c3ccccc3)n2)(c2ccccn2)N1]

Enantiomeric separation of rac-N-{[2,5-dioxo-4-(pyridin-2-yl)imidazolidin-4-yl]methyl}-2-phenyl-2H-1,2,3-triazole-4-carboxamide (30.0 mg, 79.5 μmol) using the following method
Column: Daicel Chiralpak IG 5 μm 250×20 mm
Solvent: 20% n-heptane/80% ethanol
Flow: 15 ml/min
Column temperature: 70° C.
UV-detection 220 nm
   afforded 40.0 mg (100% purity, 47% yield) of the title compound.

Chiral-HPLC (Column: 250×20 mm Daicel Chiralpak IG, 5 μm; flow: 15 ml/min; temperature: 70° C.; eluent: 20% n-heptane/80% ethanol) Rₜ: 9.684 min; >99.0% ee

LC-MS (Method 7): Rₜ=1.23 min; MS (ESIpos): m/z=378 [M+H]⁺

¹H-NMR (400 MHz, DMSO-d6) δ [ppm]: −0.149 (0.53), 0.008 (4.89), 0.146 (0.51), 1.235 (0.70), 2.366 (0.42), 2.670 (0.41), 2.710 (0.43), 4.071 (1.75), 4.087 (1.89), 4.105 (2.92), 4.121 (2.70), 4.203 (2.69), 4.219 (2.92), 4.237 (1.87), 4.253 (1.71), 7.387 (2.31), 7.399 (2.71), 7.404 (2.72), 7.417 (2.58), 7.482 (1.75), 7.501 (4.59), 7.519 (3.14), 7.543 (4.51), 7.563 (5.01), 7.601 (5.64), 7.621 (8.29), 7.640 (4.37), 7.848 (2.23), 7.853 (2.22), 7.868 (3.62), 7.872 (3.54), 7.887 (1.85), 7.891 (1.76), 8.047 (7.92), 8.066 (7.69), 8.287 (1.66), 8.450 (1.89), 8.465 (16.00), 8.480 (1.72), 8.628 (3.18), 8.638 (3.14), 10.889 (1.19).

### Example 39

### ent-N-((1R)-1-(4-Cyclopropyl-2,5-dioxoimidazolidin-4-yl)ethyl)-2-phenyl-2H-1,2,3-triazole-4-carboxamide

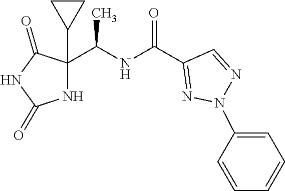 [SMILES: C[C@@H](NC(=O)c1cnn(-c2ccccc2)n1)C1(C2CC2)NC(=O)NC1=O]

To a solution of ent-5-((R)-1-aminoethyl)-5-cyclopropylimidazolidine-2,4-dione bis(2,2,2-trifluoroacetate) (25 mg, 0.061 mmol), and 2-phenyl-2H-1,2,3-triazole-4-carboxylic acid (13.7 mg, 0.073 mmol) in DMF (2 mL) were added HOBt hydrate (9.9 mg, 0.073 mmol), N-(3-dimethylaminopropyl)-N′-ethylcarbodiimide hydrochloride (13.8 mg, 0.073 mmol) and DIPEA (42 4, 0.24 mmol). The reaction mixture was stirred at room temperature over night, diluted with saturated aqueous sodium bicarbonate solution and dichloromethane followed by extraction with dichloromethane. The combined organic layers were dried over magnesium sulfate, filtered and concentrated. The residue was purified by column chromatography on silica gel (30-65% ethyl acetate in hexanes). 9.4 mg (95% purity, 65% yield) of the title compound were obtained.

LC-MS (Method 13): Rₜ=1.04 min; MS (ESIpos): m/z=355 [M+H]⁺

¹H NMR (400 MHz, DMSO-d₆) b 10.72 (s, 1H), 8.47 (s, 1H), 8.08-8.00 (m, 3H), 7.86 (s, 1H), 7.63 (t, J=7.7 Hz, 2H), 7.51 (t, J=7.3 Hz, 1H), 4.55 (m, 1H), 1.24 (s, J=6.7 Hz, 3H), 1.22 (m, 1H), 0.54-0.46 (m, 2H), 0.38 (m, 1H), 0.13 (m, 1H).

### Example 40

### ent-N-((1S)-1-(4-Cyclopropyl-2,5-dioxoimidazolidin-4-yl)ethyl)-2-phenyl-2H-1,2,3-triazole-4-carboxamide

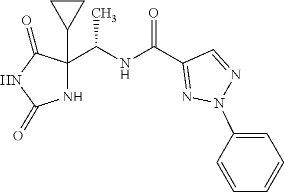 [SMILES: C[C@H](NC(=O)c1cnn(-c2ccccc2)n1)C1(C2CC2)NC(=O)NC1=O]

The reaction was carried out analogously to the preparation of ent-N-((1R)-1-(4-Cyclopropyl-2,5-dioxoimidazolidin-4-yl)ethyl)-2-phenyl-2H-1,2,3-triazole-4-carboxamide. When ent-5-((S)-1-aminoethyl)-5-cyclopropylimidazolidine-2,4-dione bis(2,2,2-trifluoroacetate) is used as starting material, 3.2 mg (95% purity, 79% yield) of the title compound were obtained.

LC-MS (Method 13): Rₜ=1.04 min; MS (ESIpos): m/z=355 [M−H]⁺

¹H NMR (400 MHz, DMSO-d₆) δ10.83 (s, 1H), 8.49 (s, 1H), 8.07-8.13 (m, 3H), 7.74 (s, 1H), 7.64 (t, J=7.7 Hz, 2H), 7.52 (t, J=7.3 Hz, 1H), 4.66 (dq, J=13.8, 6.8 Hz, 1H), 1.13 (d, J=6.7 Hz, 3H), 1.11 (m, 1H), 0.37-0.21 (m, 3H), 0.05 (m, 1H).

### Example 41

### N-{[(4R)-4-cyclopropyl-2,5-dioxoimidazolidin-4-yl]methyl}-2-(4-fluorophenyl)-1,3-oxazole-4-carboxamide

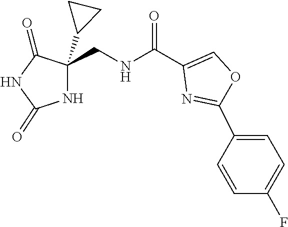 [SMILES: O=C1NC(=O)[C@](CNC(=O)c2coc(-c3ccc(F)cc3)n2)(C2CC2)N1]

The reaction was carried out analogously to the preparation of N-{[(4R)-4-cyclopropyl-2,5-dioxoimidazolidin-4-yl]methyl}-2-(4-methoxyphenyl)-2H-1,2,3-triazole-4-carboxamide. 71.0 mg (100% purity, 81% yield) of the title compound were obtained.

LC-MS (Method 7): Rₜ=1.27 min; MS (ESIpos): m/z=359 [M+H]⁺

¹H-NMR (500 MHz, DMSO-d₆) δ[ppm]: −0.007 (2.94), 0.006 (1.69), 0.120 (0.58), 0.131 (1.11), 0.140 (1.69), 0.150 (1.86), 0.159 (1.36), 0.169 (0.64), 0.316 (0.50), 0.327 (0.79), 0.334 (1.41), 0.343 (1.71), 0.351 (1.52), 0.362 (1.10), 0.370 (0.86), 0.395 (0.63), 0.404 (1.61), 0.415 (2.84), 0.425 (2.43), 0.433 (3.13), 0.445 (2.28), 0.451 (1.04), 0.463 (0.76), 1.135 (0.74), 1.146 (1.39), 1.151 (1.58), 1.162 (2.54), 1.168 (1.02), 1.173 (1.32), 1.178 (1.25), 1.189 (0.58), 1.234 (0.52), 3.611 (1.82), 3.623 (1.97), 3.638 (2.97), 3.651 (2.68), 3.717 (2.75), 3.730 (3.01), 3.744 (1.97), 3.758 (1.77), 7.411 (0.80), 7.417 (5.01), 7.421 (1.82), 7.435 (9.94), 7.448 (1.79), 7.453 (5.13), 7.459 (0.56), 7.650 (6.58), 7.984 (1.64), 7.997 (3.34), 8.009 (1.59), 8.044 (0.79), 8.050 (5.07), 8.054 (2.35), 8.061 (5.49), 8.068 (5.45), 8.074 (2.23), 8.078 (4.94), 8.743 (16.00), 10.666 (1.95).

### Example 42

### N-{[(4R)-4-cyclopropyl-2,5-dioxoimidazolidin-4-yl]methyl}-5-(4-fluorophenyl)-1,3,4-oxadiazole-2-carboxamide

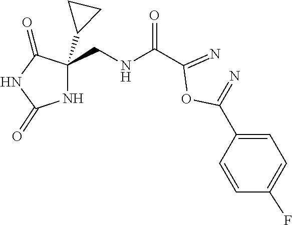 [SMILES: N=C(OC(=N)c1ccc(F)cc1)C(=O)NC[C@@]1(C2CC2)NC(=O)NC1=O]

The reaction was carried out analogously to the preparation of N-{[(4R)-4-cyclopropyl-2,5-dioxoimidazolidin-4-yl]methyl}-2-(4-methoxyphenyl)-2H-1,2,3-triazole-4-carboxamide. 5.0 mg (99% purity, 6% yield) of the title compound were obtained.

LC-MS (Method 7): Rₜ=1.16 min; MS (ESIpos): m/z=360 [M+H]⁺

¹H-NMR (500 MHz, DMSO-d6) δ [ppm]: 0.113 (1.04), 0.122 (1.85), 0.129 (2.40), 0.139 (2.68), 0.147 (2.45), 0.153 (2.03), 0.163 (1.02), 0.324 (0.91), 0.333 (1.25), 0.340 (2.55), 0.349 (2.63), 0.354 (2.81), 0.362 (2.19), 0.367 (1.77), 0.379 (1.22), 0.442 (4.38), 0.456 (8.76), 0.472 (6.91), 0.487 (2.19), 1.147 (1.25), 1.161 (2.71), 1.173 (3.52), 1.187 (2.32), 1.201 (0.81), 1.650 (1.04), 2.362 (0.50), 2.636 (0.42), 3.268 (0.83), 3.300 (9.20), 3.326 (0.60), 3.665 (1.46), 3.678 (1.64), 3.693 (6.57), 3.705 (10.61), 3.717 (6.38), 3.732 (1.51), 3.744 (1.28), 7.480 (7.97), 7.484 (3.02), 7.498 (16.00), 7.516 (8.29), 7.645 (8.96), 8.138 (8.26), 8.142 (3.99), 8.149 (8.81), 8.156 (8.81), 8.162 (3.57), 8.167 (7.84), 9.203 (2.87), 9.216 (5.76), 9.228 (2.66), 10.688 (7.56).

### Example 43

### ent-N-{[4-(2,2-dimethylcyclopropyl)-2,5-dioxoimidazolidin-4-yl]methyl}-2-phenyl-2H-1,2,3-triazole-4-carboxamide

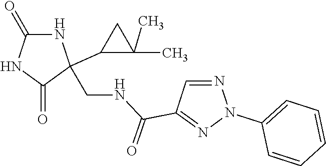 [SMILES: CC1(C)CC1C1(CNC(=O)c2cnn(-c3ccccc3)n2)NC(=O)NC1=O]

Enantiomeric separation of diamix-N-{[4-(2,2-dimethylcyclopropyl)-2,5-dioxoimidazolidin-4-yl]methyl}-2-phenyl-2H-1,2,3-triazole-4-carboxamide (229 mg, 621 μmol) using the following method
Machine: Sepiatec
Column: AD-H 5 u 250×20 mm
Solvent: 15% i-propanol/85% CO₂
Flow: 70 ml/min
Wavelength: 210 nm
System setting: backpressure: 100 bar
Temperature fraction module: 15° C.
Temperature pumphead: −2° C.
Temperature column department: 40° C.
   afforded 31.0 mg (100% purity, 14% yield) of the title compound.

Chiral-HPLC (Column: AD 15; eluent: i-propanole; flow rate: 3.0 ml/min; UV-detection: 210 nm) Rₜ: 2.932 min; >99.0% ee

LC-MS (Method 7): Rₜ=1.53 min; MS (ESIpos): m/z=369 [M+H]⁺

¹H-NMR (500 MHz, DMSO-d6) δ [ppm]: 0.397 (1.11), 0.406 (1.61), 0.415 (1.25), 0.424 (1.52), 0.450 (1.49), 0.461 (1.80), 0.470 (1.12), 0.936 (1.54), 0.948 (1.72), 0.954 (1.64), 0.966 (1.52), 0.984 (15.14), 1.089 (16.00), 3.668 (0.80), 3.680 (0.89), 3.695 (1.81), 3.707 (1.64), 3.732 (10.68), 3.745 (1.75), 3.759 (0.82), 3.773 (0.78), 7.496 (0.99), 7.511 (2.43), 7.526 (1.52), 7.588 (4.26), 7.616 (2.98), 7.633 (4.21), 7.648 (2.32), 8.062 (4.24), 8.079 (4.05), 8.450 (0.96), 8.463 (1.97), 8.477 (1.09), 8.484 (7.90), 10.764 (2.26).

### Example 44

### ent-N-{[4-(2,2-dimethylpropyl)-2,5-dioxoimidazolidin-4-yl]methyl}-2-phenyl-2H-1,2,3-triazole-4-carboxamide

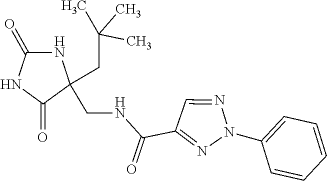 [SMILES: CC(C)(C)CC1(CNC(=O)c2cnn(-c3ccccc3)n2)NC(=O)NC1=O]

4.5 mg (6% yield) of the title compound were obtained as by-product in the synthesis of ent-N-{[4-(2,2-dimethylcyclopropyl)-2,5-dioxoimidazolidin-4-yl]methyl}-2-phenyl-2H-1,2,3-triazole-4-carboxamide.

¹H-NMR (500 MHz, DMSO-d6) δ [ppm]: 0.900 (16.00), 1.603 (0.69), 1.633 (0.92), 1.767 (0.95), 1.797 (0.69), 3.447 (0.58), 3.459 (0.52), 3.495 (0.53), 3.508 (0.57), 7.508 (0.87), 7.522 (0.54), 7.613 (1.04), 7.630 (1.50), 7.645 (0.85), 7.841 (1.41), 8.070 (1.51), 8.085 (1.47), 8.449 (0.67), 8.479 (3.06), 10.739 (0.83).

### Example 45

### N-{[(4R)-4-cyclopropyl-2,5-dioxoimidazolidin-4-yl]methyl}-2-phenyl-2H-1,2,3-triazole-4-carboxamide

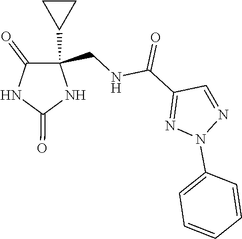 [SMILES: O=C1NC(=O)[C@](CNC(=O)c2cnn(-c3ccccc3)n2)(C2CC2)N1]

To a solution of (5R)-5-(aminomethyl)-5-cyclopropylimidazolidine-2,4-dione hydrochloride (40.0 mg, 195 μmol) and 2-phenyl-2H-1,2,3-triazole-4-carboxylic acid (36.8 mg, 195 μmol) in N,N-dimethylformamide (1 ml) was added 1-ethyl-3-(3-dimethylaminopropyl)carbodiimide hydrochloride (48.5 mg, 253 μmol), 1-hydroxybenzotriazole hydrate (38.7 mg, 253 μmol) and N,N-diisopropylethylamine (95 μl, 545 μmol). The reaction mixture was stirred at RT for 16 h. Water was added and the resulting precipitate was filtered off, washed with water and dried in vacuo. The obtained amount of product was 55 mg (100% purity, 82% of theory).

LC/MS (Method 10): Rₜ=0.93 min, MS (ESIpos): m/z=341 [M+H]⁺

¹H-NMR (400 MHz, DMSO-d6) δ [ppm]: 0.110 (0.60), 0.122 (1.17), 0.134 (1.51), 0.143 (2.04), 0.159 (1.39), 0.171 (0.74), 0.310 (0.54), 0.330 (1.49), 0.340 (1.65), 0.349 (2.15), 0.357 (1.56), 0.363 (1.25), 0.368 (1.22), 0.378 (0.97), 0.421 (2.82), 0.439 (5.15), 0.457 (4.49), 0.476 (1.65), 1.140 (0.77), 1.160 (1.59), 1.175 (2.40), 1.190 (1.55), 1.208 (0.64), 1.371 (1.33), 2.732 (0.89), 2.891 (1.02), 3.646 (1.46), 3.662 (1.66), 3.681 (3.54), 3.696 (3.28), 3.722 (3.29), 3.738 (3.43), 3.755 (1.57), 3.772 (1.45), 7.487 (1.78), 7.506 (4.58), 7.524 (3.04), 7.607 (5.85), 7.628 (11.15), 7.642 (2.77), 7.647 (4.67), 8.063 (8.44), 8.082 (7.60), 8.451 (2.09), 8.469 (16.00), 8.483 (1.99), 10.661 (3.31).

### Example 46

### N-{[(4R)-4-cyclopropyl-2,5-dioxoimidazolidin-4-yl]methyl}-3-(1-methyl-1H-pyrazol-4-yl)-1,2-oxazole-5-carboxamide

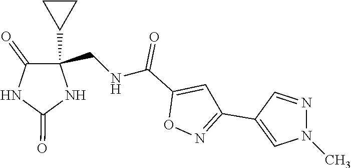 [SMILES: Cn1cc(-c2cc(C(=O)NC[C@@]3(C4CC4)NC(=O)NC3=O)on2)cn1]

To a solution of (5R)-5-(aminomethyl)-5-cyclopropylimidazolidine-2,4-dione hydrochloride (60.0 mg, 292 μmol) and 3-(1-methyl-1H-pyrazol-4-yl)-1,2-oxazole-5-carboxylic acid (62.0 mg, 321 μmol) in N,N-dimethylformamide (1 ml) was added 1-ethyl-3-(3-dimethylaminopropyl)carbodiimide hydrochloride (72.7 mg, 379 μmol), (1.3 eq) 1-hydroxybenzotriazole hydrate (58.1 mg, 379 μmol) and N,N-diisopropylethylamine (140 μl, 817 μmol). The reaction mixture was stirred at RT for 16 h. The reaction was partitioned between ethyl acetate and a saturated solution of NaHCO₃. The water layer was washed with ethyl acetate, dried over magnesium sulfate, filtered and concentrated. Acetonitrile was added to the crude and the resulting precipitate was filtered off and dried in vacuo to afford the product. The obtained amount of product of product was 35 mg (100% purity, 35% of theory).

LC/MS (Method 7): Rₜ=0.84 min, MS (ESIpos): m/z=345 [M+H]⁺

¹H-NMR (500 MHz, DMSO-d₆) δ[ppm]: 0.116 (0.71), 0.125 (1.04), 0.135 (1.08), 0.144 (0.82), 0.333 (0.73), 0.345 (0.93), 0.354 (0.89), 0.361 (0.58), 0.370 (0.42), 0.422 (0.75), 0.439 (1.51), 0.450 (1.55), 0.455 (1.57), 0.466 (1.24), 0.476 (0.64), 1.135 (0.76), 1.140 (0.83), 1.151 (1.30), 1.161 (0.76), 1.167 (0.65), 3.600 (0.74), 3.612 (0.82), 3.628 (1.62), 3.640 (1.45), 3.668 (1.50), 3.682 (1.57), 3.695 (0.79), 3.709 (0.72), 3.904 (16.00), 7.373 (6.69), 7.599 (3.81), 7.932 (5.28), 8.309 (5.13), 8.872 (0.96), 8.884 (1.77), 8.897 (0.89), 10.651 (2.32).

### Example 47

### N-{[(4R)-4-cyclopropyl-2,5-dioxoimidazolidin-4-yl]methyl}-2-(pyridin-2-yl)-2H-1,2,3-triazole-4-carboxamide

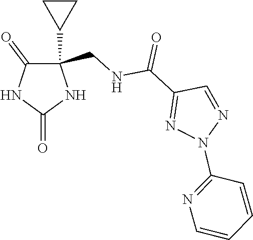 [SMILES: O=C1NC(=O)[C@](CNC(=O)c2cnn(-c3ccccn3)n2)(C2CC2)N1]

To a solution of (5R)-5-(aminomethyl)-5-cyclopropylimidazolidine-2,4-dione hydrochloride (100.0 mg, 486 μmol) and 2-(pyridin-2-yl)-2H-1,2,3-triazole-4-carboxylic acid (92.4 mg, 486 μmol) in N,N-dimethylformamide (2.5 ml) was added 1-ethyl-3-(3-dimethylaminopropyl)carbodiimide hydrochloride (121.2 mg, 632 μmol), 1-hydroxybenzotriazole hydrate (96.8 mg, 632 μmol) and N,N-diisopropylethylamine (237 μl, 1.36 mmol). The reaction mixture was stirred at RT for 16 h. The reaction was partitioned between ethyl acetate and water. The organic layer was washed with water, dried over magnesium sulfate, filtered and concentrated. The mixture was filtered through a ion exchange filter, evaporated and dried in vacuo to afford the desired product. The obtained amount of product was 43 mg (100% purity, 26% of theory).

LC/MS (Method 7): Rₜ=0.90 min, MS (ESIpos): m/z=342 [M+H]⁺

¹H-NMR (600 MHz, DMSO-d6) δ [ppm]: 0.005 (2.69), 0.108 (0.65), 0.115 (1.29), 0.123 (1.51), 0.127 (2.02), 0.135 (1.73), 0.141 (1.52), 0.149 (0.80), 0.316 (0.58), 0.323 (0.77), 0.329 (1.78), 0.337 (1.84), 0.341 (1.91), 0.348 (1.65), 0.352 (1.31), 0.362 (0.94), 0.418 (3.02), 0.430 (5.81), 0.443 (4.95), 0.454 (1.70), 1.145 (0.86), 1.158 (1.86), 1.168 (2.61), 1.179 (1.70), 1.190 (0.75), 3.679 (1.08), 3.690 (1.26), 3.702 (4.57), 3.713 (7.80), 3.724 (4.46), 3.736 (1.21), 3.747 (1.10), 7.584 (2.65), 7.592 (3.04), 7.595 (3.09), 7.603 (2.90), 7.640 (6.39), 8.084 (3.81), 8.098 (6.52), 8.128 (3.02), 8.131 (3.07), 8.140 (3.49), 8.143 (3.70), 8.154 (1.77), 8.157 (1.77), 8.524 (16.00), 8.562 (2.09), 8.573 (4.23), 8.583 (2.08), 8.628 (3.62), 8.634 (3.64), 10.680 (5.62).

### Example 48

### rac-N-[(2,5-dioxo-4-(propan-2-yl)imidazolidin-4-yl)methyl]-2-phenyl-2H-1,2,3-triazole-4-carboxamide

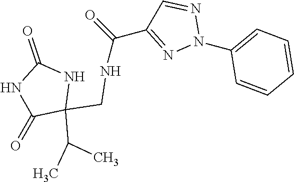 [SMILES: CC(C)C1(CNC(=O)c2cnn(-c3ccccc3)n2)NC(=O)NC1=O]

To a solution of rac-5-(aminomethyl)-5-(propan-2-yl)imidazolidine-2,4-dione (100.0 mg, 584 μmol) and 2-phenyl-2H-1,2,3-triazole-4-carboxylic acid (110.5 mg, 584 μmol) in N,N-dimethylformamide (2.4 ml) was added 1-ethyl-3-(3-dimethylaminopropyl)carbodiimide hydrochloride (145.6 mg, 759 μmol), 1-hydroxybenzotriazole hydrate (116.3 mg, 759 μmol) and N,N-diisopropylethylamine (284 μl, 1.6 mmol). The reaction mixture was stirred at RT for 16 h. Water was added in to the mixture and the resulting precipitate was filtered off, washed with water and dried in vacuo. The obtained amount of product was was 157 mg (100% purity, 79% of theory).

LC-MS (Method 7): Rₜ=1.25 min; MS (ESIpos): m/z=343 [M+H]⁺

¹H-NMR (500 MHz, DMSO-d6) δ [ppm]: 0.006 (1.40), 0.848 (15.82), 0.861 (15.83), 0.956 (15.67), 0.969 (15.81), 1.979 (0.99), 1.992 (2.44), 2.006 (3.20), 2.019 (2.25), 2.033 (0.83), 2.074 (2.02), 3.604 (1.07), 3.617 (1.22), 3.631 (4.30), 3.645 (6.91), 3.658 (4.10), 3.674 (1.09), 3.686 (0.97), 7.491 (2.10), 7.506 (5.17), 7.521 (3.22), 7.609 (6.28), 7.625 (9.11), 7.641 (4.89), 7.810 (6.64), 8.056 (9.26), 8.072 (8.77), 8.315 (2.04), 8.327 (3.96), 8.340 (1.88), 8.464 (16.00), 10.661 (5.67).

### Example 49

### rac-[(4-tert-butyl-2,5-dioxoimidazolidin-4-yl)methyl]-2-phenyl-2H-1,2,3-triazole-4-carboxamide

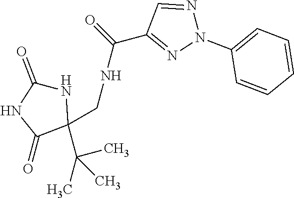 [SMILES: CC(C)(C)C1(CNC(=O)c2cnn(-c3ccccc3)n2)NC(=O)NC1=O]

To a solution of rac-5-(aminomethyl)-5-tert-butylimidazolidine-2,4-dione (50.0 mg, 267 μmol) and 2-phenyl-2H-1,2,3-triazole-4-carboxylicacid (46.0 mg, 243 μmol) in N,N-dimethylformamide (2.2 ml) was added 1-ethyl-3-(3-dimethylaminopropyl)carbodiimide hydrochloride (60.5 mg, 316 μmol), (1.3 eq) 1-hydroxybenzotriazole hydrate (48.4 mg, 316 μmol) and N,N-diisopropylethylamine(120 μl, 680 μmol). The reaction mixture was stirred at RT for 16 h and then the mixture was purified by preparative HPLC (gradient acetonitrile/water with 0.1% trifluoroacetic acid). Evaporation of the combined product fractions yielded the title compound. The obtained amount of product was 34 mg (100% purity, 36% of theory).

LC-MS (Method 7): Rₜ=1.36 min; MS (ESIpos): m/z=357 [M+H]⁺

¹H-NMR (500 MHz, DMSO-d₆) δ[ppm]: −0.007 (0.43), 1.022 (16.00), 2.074 (0.66), 3.688 (0.55), 3.700 (0.52), 3.779 (0.55), 3.792 (0.60), 3.806 (0.40), 7.491 (0.40), 7.506 (0.97), 7.521 (0.63), 7.610 (2.31), 7.626 (1.66), 7.638 (0.43), 7.641 (0.95), 8.045 (1.59), 8.047 (1.66), 8.062 (1.65), 8.065 (1.19), 8.288 (0.68), 8.447 (3.47), 10.599 (1.00).

### Example 50

### rac-N-[(4-cyclopentyl-2,5-dioxoimidazolidin-4-yl)methyl]-2-phenyl-2H-1,2,3-triazole-4-carboxamide

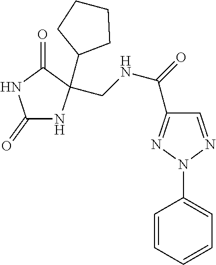 [SMILES: O=C1NC(=O)C(CNC(=O)c2cnn(-c3ccccc3)n2)(C2CCCC2)N1]

To a solution of rac-5-(aminomethyl)-5-cyclopentylimidazolidine-2,4-dione hydrochloride (50.0 mg, 214 μmol) and 2-phenyl-2H-1,2,3-triazole-4-carboxylic acid (40.5 mg, 214 μmol) in N,N-dimethylformamide (100 μl, 600 μmol) was added 1-ethyl-3-(3-dimethylaminopropyl)carbodiimide hydrochloride (53.3 mg, 278 μmol), 1-hydroxybenzotriazole hydrate (42.6 mg, 278 μmol) and N,N-diisopropylethylamine (100 μl, 600 μmol). The reaction mixture was stirred at RT for 16 h and then the mixture was purified by preparative HPLC (gradient acetonitrile/water with 0.1% trifluoroacetic acid). Evaporation of the combined product fractions yielded the title compound. The obtained amount of product was 25 mg (100% purity, 31% of theory).

LC-MS (Method 7): Rₜ=1.46 min; MS (ESIpos): m/z=369 [M+H]⁺

¹H-NMR (500 MHz, DMSO-d₆) δ[ppm]: −0.007 (2.42), 0.006 (1.63), 1.214 (0.86), 1.223 (1.02), 1.233 (1.09), 1.241 (1.00), 1.339 (0.82), 1.359 (1.09), 1.374 (0.87), 1.397 (0.44), 1.490 (3.50), 1.506 (2.70), 1.523 (2.14), 1.540 (1.74), 1.548 (1.96), 1.556 (1.89), 1.565 (1.97), 1.771 (1.23), 1.787 (1.05), 2.073 (6.51), 2.180 (0.48), 2.196 (1.14), 2.214 (1.58), 2.231 (1.04), 3.615 (6.91), 3.628 (6.92), 7.490 (1.79), 7.505 (4.33), 7.520 (2.79), 7.609 (5.41), 7.626 (7.21), 7.637 (1.80), 7.641 (4.19), 7.845 (5.15), 8.055 (7.04), 8.057 (7.38), 8.072 (7.37), 8.074 (5.27), 8.381 (1.59), 8.394 (3.29), 8.406 (1.51), 8.462 (16.00), 10.654 (4.53).

### Example 51

### rac-N-[(4-butyl-2,5-dioxoimidazolidin-4-yl)methyl]-2-phenyl-2H-1,2,3-triazole-4-carboxamide

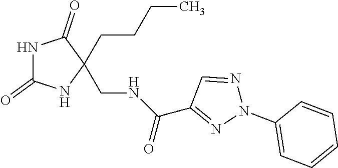 [SMILES: CCCCC1(CNC(=O)c2cnn(-c3ccccc3)n2)NC(=O)NC1=O]

To a solution of rac-5-(aminomethyl)-5-butylimidazolidine-2,4-dione (40.0 mg, 216 μmol) and 2-phenyl-2H-1,2,3-triazole-4-carboxylic acid (44.9 mg, 238 μmol) in N,N-dimethylformamide (1.0 ml) was added 1-ethyl-3-(3-dimethylaminopropyl)carbodiimide hydrochloride (53.8 mg, 281 μmol), 1-hydroxybenzotriazole hydrate (43.0 mg, 281 μmol) and N,N-diisopropylethylamine (105 μl, 605 μmol). The reaction mixture was stirred at RT for 2 h and then the mixture was purified by preparative HPLC (gradient acetonitrile/water with 0.1% trifluoroacetic acid). Evaporation of the combined product fractions yielded the title compound. The obtained amount of product was 33 mg (100% purity, 43% of theory).

LC-MS (Method 7): Rₜ=1.45 min; MS (ESIpos): m/z=357 [M+H]⁺

¹H-NMR (500 MHz, DMSO-d6) δ [ppm]: −0.120 (0.42), −0.006 (5.65), 0.007 (3.17), 0.117 (0.40), 0.837 (5.51), 0.852 (12.53), 0.866 (6.41), 1.020 (0.45), 1.030 (0.79), 1.036 (0.85), 1.044 (1.23), 1.054 (1.10), 1.068 (0.94), 1.225 (0.68), 1.240 (1.86), 1.254 (3.94), 1.259 (3.54), 1.265 (4.44), 1.273 (3.56), 1.304 (0.50), 1.570 (0.74), 1.578 (0.83), 1.598 (1.80), 1.621 (1.38), 1.629 (1.06), 1.650 (1.07), 1.660 (1.06), 1.672 (1.53), 1.700 (0.76), 3.510 (1.03), 3.523 (1.22), 3.537 (3.51), 3.550 (3.55), 3.556 (3.41), 3.569 (3.30), 3.584 (1.09), 3.597 (1.01), 7.490 (1.89), 7.505 (4.44), 7.520 (2.88), 7.610 (5.56), 7.627 (7.48), 7.638 (1.92), 7.642 (4.25), 7.781 (5.40), 8.066 (7.16), 8.068 (7.70), 8.083 (7.64), 8.085 (5.58), 8.473 (16.00), 8.496 (1.71), 8.509 (3.40), 8.522 (1.60), 10.678 (4.59).

### Example 52

### rac-N-[4-cyclobutyl-2,5-dioxoimidazolidin-4-yl)methyl]-2-phenyl-2H-1,2,3-triazole-4-carboxamide

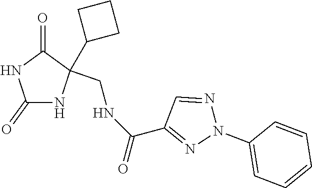 [SMILES: O=C1NC(=O)C(CNC(=O)c2cnn(-c3ccccc3)n2)(C2CCC2)N1]

To a solution of rac-5-(aminomethyl)-5-cyclobutylimidazolidine-2,4-dione hydrochloride (100.0 mg, 455 μmol) and 2-phenyl-2H-1,2,3-triazole-4-carboxylic acid (86.1 mg, 455 μmol) in N,N-dimethylformamide (2.5 ml) was added 1-ethyl-3-(3-dimethylaminopropyl)carbodiimide hydrochloride (113.4 mg, 592 μmol), 1-hydroxybenzotriazole hydrate (90.6 mg, 592 μmol) and N,N-diisopropylethylamine (222 μl, 1.28 mmol). The reaction mixture was stirred at RT for 16 h. Water was added in to the mixture and the resulting precipitate was filtered off, washed with water and dried under vacuo afforded to afford the product. The obtained amount of product was 132 mg (100% purity, 82% of theory).

LC-MS (Method 9): Rₜ=1.06 min; MS (ESIpos): m/z=355 [M+H]⁺

¹H-NMR (400 MHz, DMSO-d6) δ [ppm]: −0.008 (2.37), 0.008 (2.13), 1.669 (1.44), 1.706 (1.29), 1.731 (1.54), 1.747 (1.33), 1.768 (2.12), 1.791 (2.41), 1.811 (2.37), 1.832 (1.42), 1.852 (0.92), 1.905 (1.94), 1.928 (2.71), 1.951 (1.92), 2.675 (0.71), 2.700 (1.41), 2.720 (2.13), 2.731 (2.11), 2.743 (1.31), 2.891 (1.81), 3.452 (0.94), 3.468 (1.10), 3.487 (3.61), 3.506 (4.33), 3.523 (3.49), 3.541 (1.00), 3.558 (0.93), 7.487 (1.76), 7.506 (4.63), 7.524 (3.16), 7.607 (5.67), 7.628 (8.11), 7.647 (4.31), 7.990 (5.77), 8.058 (8.03), 8.077 (7.90), 8.390 (1.62), 8.406 (3.16), 8.422 (1.52), 8.461 (16.00), 10.661 (4.60).

### Example 53

### rac-N-[(4-cyclohexyl-2,5-dioxoimidazolidin-4-yl)methyl]-2-phenyl-2H-1,2,3-triazole-4-carboxamide

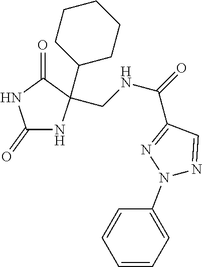 [SMILES: O=C1NC(=O)C(CNC(=O)c2cnn(-c3ccccc3)n2)(C2CCCCC2)N1]

To a solution of rac-5-(aminomethyl)-5-cyclohexylimidazolidine-2,4-dione hydrochloride (50.0 mg, 202 μmol) and 2-phenyl-2H-1,2,3-triazole-4-carboxylic acid (38.2 mg, 202 μmol) in N,N-dimethylformamide (2.0 ml) was added 1-ethyl-3-(3-dimethylaminopropyl)carbodiimide hydrochloride (50.3 mg, 262 μmol), 1-hydroxybenzotriazole hydrate (40.2 mg, 262 μmol) and N,N-diisopropylethylamine (98 μl, 570 μmol). The reaction mixture was stirred at RT for 16 h and then the mixture was purified by preparative HPLC (gradient acetonitrile/water with 0.1% trifluoroacetic acid). Evaporation of the combined product fractions yielded the title compound. The obtained amount of product was 18 mg (98% purity, 23% of theory).

LC-MS (Method 7): Rₜ=1.53 min; MS (ESIpos): m/z=383 [M+H]⁺

¹H-NMR (600 MHz, DMSO-d6) δ [ppm]: 1.027 (1.36), 1.048 (3.98), 1.069 (4.72), 1.089 (2.16), 1.125 (2.01), 1.145 (5.18), 1.163 (4.96), 1.179 (3.43), 1.200 (2.27), 1.486 (2.54), 1.502 (2.99), 1.600 (2.78), 1.620 (2.60), 1.653 (1.88), 1.672 (3.19), 1.690 (4.50), 1.706 (2.34), 1.736 (2.71), 1.756 (2.34), 1.810 (2.64), 1.831 (2.47), 2.078 (2.14), 3.593 (2.21), 3.604 (2.43), 3.616 (4.37), 3.627 (4.07), 3.660 (4.09), 3.670 (4.33), 3.683 (2.32), 3.693 (2.14), 7.495 (2.82), 7.508 (6.49), 7.520 (4.15), 7.614 (7.19), 7.627 (12.77), 7.640 (6.19), 7.821 (10.47), 8.059 (12.44), 8.072 (11.76), 8.331 (2.89), 8.342 (5.84), 8.352 (2.89), 8.472 (16.00), 10.667 (9.33).

### Example 54

### rac-N-[(4-(cyclopropylmethyl)-2,5-dioxoimidazolidin-4-yl)methyl}]-2-phenyl-2H-1,2,3-triazole-4-carboxamide

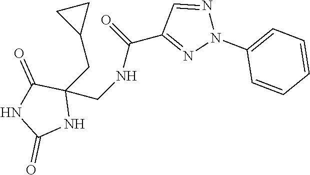 [SMILES: O=C1NC(=O)C(CNC(=O)c2cnn(-c3ccccc3)n2)(CC2CC2)N1]

To a solution of rac-5-(aminomethyl)-5-(cyclopropylmethyl)imidazolidine-2,4-dione hydrochloride (50.0 mg, 225 μmol) and 2-phenyl-2H-1,2,3-triazole-4-carboxylic acid (38.8 mg, 205 μmol) in N,N-dimethylformamide (1.9 ml) was added 1-ethyl-3-(3-dimethylaminopropyl)carbodiimide hydrochloride (51.1 mg, 266 μmol), 1-hydroxybenzotriazole hydrate (40.8 mg, 266 μmol) and N,N-diisopropylethylamine (100 μl, 570 μmol). The reaction mixture was stirred at RT for 16 h and then the mixture was purified by preparative HPLC (gradient acetonitrile/water with 0.1% trifluoroacetic acid). Evaporation of the combined product fractions yielded the title compound. The obtained amount of product was 13 mg (95% purity, 16% of theory).

LC-MS (Method 7): Rₜ=1.33 min; MS (ESIpos): m/z=355 [M+H]⁺

¹H-NMR (500 MHz, DMSO-d6) δ [ppm]: 1.678 (0.83), 1.688 (1.02), 1.706 (2.09), 1.715 (2.01), 1.729 (2.38), 1.739 (2.26), 1.749 (1.78), 1.759 (2.26), 1.772 (2.13), 1.781 (2.45), 1.799 (1.08), 1.809 (1.18), 1.824 (0.87), 1.837 (1.21), 1.863 (1.78), 1.876 (1.49), 1.888 (1.02), 2.033 (1.39), 2.045 (1.63), 2.054 (1.66), 2.073 (1.59), 3.523 (1.63), 3.536 (1.84), 3.551 (4.40), 3.563 (4.19), 3.575 (4.10), 3.588 (4.03), 3.602 (1.58), 3.616 (1.41), 4.940 (3.90), 4.958 (4.01), 5.002 (3.79), 5.005 (3.52), 5.036 (4.08), 5.739 (1.02), 5.751 (1.99), 5.759 (1.23), 5.764 (1.29), 5.772 (2.57), 5.785 (2.45), 5.793 (1.08), 5.798 (1.09), 5.806 (1.61), 5.819 (0.73), 7.491 (2.51), 7.506 (5.52), 7.521 (3.55), 7.611 (6.88), 7.627 (9.87), 7.642 (5.09), 7.819 (7.46), 8.069 (10.50), 8.085 (9.30), 8.475 (16.00), 8.518 (1.36), 8.537 (2.51), 8.549 (4.54), 8.562 (2.12), 10.720 (6.49).

### Example 55

### rac-N-[(2,5-dioxo-4-propylimidazolidin-4-yl)methyl]-2-phenyl-2H-1,2,3-triazole-4-carboxamide

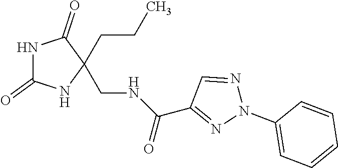 [SMILES: CCCC1(CNC(=O)c2cnn(-c3ccccc3)n2)NC(=O)NC1=O]

To a solution of rac-5-(aminomethyl)-5-propylimidazolidine-2,4-dione hydrochloride (40.0 mg, 193 μmol) and 2-phenyl-2H-1,2,3-triazole-4-carboxylic acid (40.1 mg, 212 μmol) in N,N-dimethylformamide (1.0 ml) was added 1-ethyl-3-(3-dimethylaminopropyl)carbodiimide hydrochloride (48.0 mg, 250 μmol), 1-hydroxybenzotriazole hydrate (38.3 mg, 250 μmol) and N,N-diisopropylethylamine (94 μl, 539 μmol). The reaction mixture was stirred at RT for 16 h and then the mixture was purified by preparative HPLC (gradient acetonitrile/water with 0.1% trifluoroacetic acid). Evaporation of the combined product fractions yielded the title compound.

The obtained amount of product was 11 mg (98% purity, 17% of theory).

LC-MS (Method 7): Rₜ=1.32 min; MS (ESIpos): m/z=343 [M+H]⁺

¹H-NMR (600 MHz, DMSO-d6) δ [ppm]: 0.852 (7.15), 0.865 (16.00), 0.877 (8.21), 1.065 (0.49), 1.074 (0.91), 1.086 (1.47), 1.095 (1.46), 1.106 (1.51), 1.118 (0.92), 1.127 (0.55), 1.278 (0.55), 1.287 (0.91), 1.298 (1.49), 1.307 (1.32), 1.319 (1.43), 1.331 (0.80), 1.339 (0.47), 1.557 (0.91), 1.565 (1.02), 1.580 (2.04), 1.587 (1.84), 1.600 (1.70), 1.608 (1.38), 1.628 (1.62), 1.636 (1.79), 1.648 (1.78), 1.655 (1.80), 1.670 (0.93), 1.678 (0.75), 3.502 (6.00), 3.513 (4.13), 3.525 (4.77), 3.536 (4.06), 3.563 (3.60), 3.574 (3.70), 3.585 (1.95), 3.596 (1.81), 7.496 (2.07), 7.508 (4.80), 7.520 (2.99), 7.616 (5.42), 7.629 (9.31), 7.642 (4.61), 7.821 (7.24), 8.073 (9.26), 8.086 (8.55), 8.484 (12.56), 8.537 (2.13), 8.548 (4.25), 8.559 (2.07), 10.710 (6.17).

### Example 56

### N-{[(4R)-4-cyclopropyl-2,5-dioxoimidazolidin-4-yl]methyl}-1-phenyl-1H-pyrazole-4-carboxamide

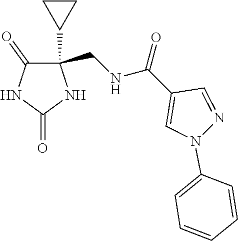 [SMILES: O=C1NC(=O)[C@](CNC(=O)c2cnn(-c3ccccc3)c2)(C2CC2)N1]

To a solution of (5R)-5-(aminomethyl)-5-cyclopropylimidazolidine-2,4-dione hydrochloride (40.0 mg, 195 μmol) and 1-phenyl-1H-pyrazole-4-carboxylic acid (36.6 mg, 195 μmol) in N,N-dimethylformamide (1.0 ml) was added 1-ethyl-3-(3-dimethylaminopropyl)carbodiimide hydrochloride (48.5 mg, 253 μmol), 1-hydroxybenzotriazole hydrate (38.7 mg, 253 μmol) and N,N-diisopropylethylamine (95 μl, 545 μmol). The reaction mixture was stirred at RT for 16 h. The reaction was partitioned between ethyl acetate and water. The organic layer was washed with water, dried over magnesium sulfate, filtered and concentrated. The obtained amount of product was 55 mg (97% purity, 81% of theory).

LC-MS (Method 7): Rₜ=1.11 min; MS (ESIpos): m/z=340 [M+H]⁺

¹H-NMR (600 MHz, DMSO-d₆) δ[ppm]: 0.005 (0.47), 0.111 (0.69), 0.120 (1.60), 0.127 (2.63), 0.136 (2.75), 0.144 (1.95), 0.152 (0.89), 0.318 (0.60), 0.325 (0.96), 0.328 (0.98), 0.333 (1.69), 0.341 (2.18), 0.349 (1.94), 0.356 (1.45), 0.363 (0.98), 0.414 (0.80), 0.422 (1.54), 0.429 (1.61), 0.431 (1.70), 0.437 (2.53), 0.446 (1.96), 0.451 (1.28), 0.463 (1.31), 0.472 (2.12), 0.480 (2.64), 0.489 (2.56), 0.496 (1.75), 0.505 (0.65), 0.983 (0.55), 1.125 (0.91), 1.133 (1.78), 1.138 (1.94), 1.142 (1.35), 1.147 (3.32), 1.152 (1.37), 1.156 (1.83), 1.161 (1.71), 1.169 (0.80), 2.732 (2.24), 2.891 (2.46), 3.478 (2.66), 3.487 (3.02), 3.501 (3.40), 3.510 (2.99), 3.774 (3.02), 3.786 (3.22), 3.797 (2.82), 3.810 (2.65), 7.359 (2.38), 7.371 (5.54), 7.383 (3.28), 7.521 (6.31), 7.535 (9.83), 7.547 (13.09), 7.833 (9.86), 7.846 (8.91), 8.150 (15.81), 8.206 (2.32), 8.217 (3.39), 8.227 (2.34), 8.953 (16.00), 10.625 (6.09).

### Example 57

### diamix-N-[4-(1-hydroxypropan-2-yl)-2,5-dioxoimidazolidin-4-yl)methyl]-2-phenyl-2H-1,2,3-triazole-4-carboxamide

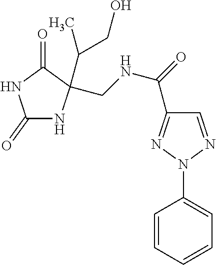 [SMILES: CC(CO)C1(CNC(=O)c2cnn(-c3ccccc3)n2)NC(=O)NC1=O]

To a solution of rac-5-(aminomethyl)-5-(1-hydroxypropan-2-yl)imidazolidine-2,4-dione hydrochloride (50.0 mg, 224 μmol) and 2-phenyl-2H-1,2,3-triazole-4-carboxylic acid (42.3 mg, 224 μmol) in N,N-dimethylformamide (2 ml) was added 1-ethyl-3-(3-dimethylaminopropyl)carbodiimide hydrochloride (55.7 mg, 291 μmol), 1-hydroxybenzotriazole hydrate (44.5 mg, 291 μmol) and N,N-diisopropylethylamine (110 μl, 630 μmol). The reaction mixture was stirred at RT for 16 h and the 4 h at 50° C. The mixture was purified by preparative HPLC (gradient acetonitrile/water with 0.1% trifluoroacetic acid). Evaporation of the combined product fractions yielded the title compound which was crystallized after that with acetonitrile, filtered and dried in vacuo. The obtained amount of product was 5 mg (95% purity, 6% of theory).

LC-MS (Method 7): Rₜ=1.02 min; MS (ESIpos): m/z=359 [M+H]⁺

¹H-NMR (500 MHz, DMSO-d6) δ [ppm]: 0.849 (15.95), 0.862 (16.00), 0.946 (1.25), 0.959 (1.22), 1.976 (1.48), 1.989 (2.65), 2.001 (2.62), 2.014 (1.47), 2.074 (4.38), 3.356 (1.42), 3.367 (2.16), 3.378 (2.88), 3.388 (2.42), 3.399 (1.48), 3.570 (1.64), 3.581 (2.98), 3.592 (3.00), 3.602 (2.73), 3.612 (3.43), 3.625 (2.52), 3.639 (3.07), 3.653 (2.73), 3.788 (2.75), 3.800 (2.90), 3.815 (2.21), 3.828 (2.04), 4.666 (0.51), 4.796 (3.11), 4.806 (6.27), 4.816 (2.85), 7.490 (2.25), 7.505 (5.27), 7.520 (3.39), 7.608 (6.51), 7.624 (9.73), 7.640 (10.90), 7.850 (0.53), 8.055 (9.46), 8.072 (9.08), 8.368 (2.09), 8.381 (3.97), 8.393 (1.93), 8.456 (14.98), 8.465 (1.53), 10.611 (0.51), 10.657 (5.78).

### Example 58

### rac-N-[4-tert-butyl-2,5-dioxoimidazolidin-4-yl)methyl]-1-phenyl-1H-pyrazole-4-carboxamide

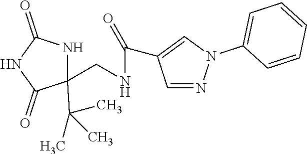 [SMILES: CC(C)(C)C1(CNC(=O)c2cnn(-c3ccccc3)c2)NC(=O)NC1=O]

To a solution of rac-5-(aminomethyl)-5-tert-butylimidazolidine-2,4-dione (50.0 mg, 267 μmol) and 1-phenyl-1H-pyrazole-4-carboxylic acid (45.7 mg, 243 μmol) in N,N-dimethylformamide (2.2 ml) was added 1-ethyl-3-(3-dimethylaminopropyl)carbodiimide hydrochloride (60.5 mg, 316 μmol), 1-hydroxybenzotriazole hydrate (48.4 mg, 316 μmol) and N,N-diisopropylethylamine (120 μl, 680 μmol). The reaction mixture was stirred at RT for 16 h and then the mixture was purified by preparative HPLC (gradient acetonitrile/water with 0.1% trifluoroacetic acid). Evaporation of the combined product fractions yielded the title compound. The obtained amount of product was 59 mg (100% purity, 62% of theory).

LC-MS (Method 7): Rₜ=1.25 min; MS (ESIpos): m/z=356 [M+H]⁺

¹H-NMR (400 MHz, DMSO-d6) δ [ppm]: −0.008 (0.54), 0.008 (0.46), 1.019 (16.00), 2.073 (1.20), 3.497 (0.43), 3.517 (0.50), 3.532 (0.45), 3.814 (0.45), 3.831 (0.46), 7.350 (0.41), 7.369 (1.01), 7.387 (0.63), 7.434 (1.15), 7.511 (1.16), 7.532 (1.69), 7.551 (1.03), 7.812 (1.78), 7.831 (1.59), 8.115 (2.82), 8.142 (0.61), 8.910 (2.67), 10.554 (1.04).

### Example 59

### N-{[(4R)-4-cyclopropyl-2,5-dioxoimidazolidin-4-yl]methyl}-2-phenyl-1,3-oxazole-5-carboxamide

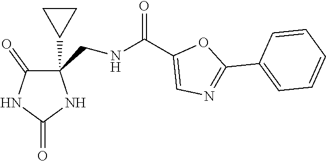 [SMILES: O=C1NC(=O)[C@](CNC(=O)c2cnc(-c3ccccc3)o2)(C2CC2)N1]

To a solution of (5R)-5-(aminomethyl)-5-cyclopropylimidazolidine-2,4-dione hydrochloride (40.0 mg, 195 μmol) and 2-phenyl-1,3-oxazole-5-carboxylic acid (33.5 mg, 177 μmol) in N,N-dimethylformamide (1.6 ml) was added 1-ethyl-3-(3-dimethylaminopropyl)carbodiimide hydrochloride (44.1 mg, 230 μmol), 1-hydroxybenzotriazole hydrate (35.2 mg, 230 μmol) and N,N-diisopropylethylamine (86 μl, 500 μmol). The reaction mixture was stirred at RT for 16 h and then the mixture was purified by preparative HPLC (gradient acetonitrile/water with 0.1% trifluoroacetic acid). Evaporation of the combined product fractions yielded the title compound. The obtained amount of product was 44 mg (100% purity, 66% of theory).

LC-MS (Method 7): Rₜ=1.13 min; MS (ESIpos): m/z=341 [M+H]⁺

¹H-NMR (400 MHz, DMSO-d6) δ [ppm]: −0.008 (1.33), 0.008 (1.26), 0.126 (0.97), 0.136 (1.67), 0.149 (1.71), 0.160 (1.25), 0.174 (0.57), 0.339 (1.06), 0.354 (1.37), 0.360 (1.08), 0.365 (1.38), 0.375 (0.97), 0.386 (0.68), 0.421 (0.49), 0.432 (1.15), 0.453 (2.27), 0.468 (2.42), 0.475 (2.18), 0.489 (1.97), 0.503 (0.93), 1.132 (0.57), 1.146 (1.16), 1.152 (1.26), 1.166 (2.20), 1.179 (1.15), 1.186 (1.02), 1.200 (0.47), 3.545 (1.54), 3.560 (1.75), 3.580 (2.34), 3.594 (2.08), 3.735 (2.15), 3.753 (2.25), 3.769 (1.67), 3.787 (1.61), 7.576 (1.72), 7.586 (5.80), 7.590 (6.34), 7.598 (11.21), 7.603 (14.42), 7.916 (16.00), 8.099 (4.69), 8.110 (4.78), 8.118 (5.28), 8.124 (4.21), 8.642 (1.32), 8.658 (2.38), 8.674 (1.26), 10.647 (3.88).

### Example 60

### N-{[(4R)-4-cyclopropyl-2,5-dioxoimidazolidin-4-yl]methyl}-2-(pyridin-2-yl)-2H-1,2,3-triazole-4-carboxamide

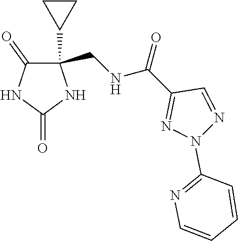 [SMILES: O=C1NC(=O)[C@](CNC(=O)c2cnn(-c3ccccn3)n2)(C2CC2)N1]

To a solution of (5R)-5-(aminomethyl)-5-cyclopropylimidazolidine-2,4-dione hydrochloride and 2-(pyridin-2-yl)-2H-1,2,3-triazole-4-carboxylic acid (92.5 mg, 486 μmol) in N,N-dimethylformamide (0.63 ml) was added 1-ethyl-3-(3-dimethylaminopropyl)carbodiimide hydrochloride (121 mg, 632 μmol), 1-hydroxybenzotriazole hydrate (96.8 mg, 632 μmol) and N,N-diisopropylethylamine (240 μl, 136 mmol). The reaction mixture was stirred at RT for 16 h.

The mixture was partitioned between ethyl acetate and water. The organic layer was washed with water, dried over magnesium sulfate, filtered and concentrated. The crude was solved in acetonitrile and filtered through an ion exchange filter and then dried in vacuo. The obtained amount of product was 43 mg (100% purity, 26% of theory).

LC-MS (Method 7): Rₜ=0.90 min; MS (ESIpos): m/z=342 [M+H]⁺

¹H-NMR (400 MHz, DMSO-d6) δ [ppm]: −0.008 (1.16), 0.132 (1.38), 0.162 (0.64), 0.308 (1.21), 0.326 (1.45), 0.357 (0.76), 0.394 (2.23), 0.412 (4.22), 0.431 (3.37), 0.447 (1.07), 1.125 (0.59), 1.145 (1.38), 1.160 (1.85), 1.176 (1.21), 1.192 (0.47), 2.096 (1.35), 2.328 (1.04), 2.524 (2.92), 2.671 (0.99), 3.691 (4.88), 3.707 (4.76), 3.926 (1.04), 7.512 (1.68), 7.573 (2.08), 7.576 (2.25), 7.584 (2.16), 7.588 (2.58), 7.590 (2.59), 7.593 (2.44), 7.603 (2.35), 7.606 (2.44), 8.074 (2.63), 8.095 (5.81), 8.118 (3.01), 8.122 (3.03), 8.136 (2.77), 8.140 (3.11), 8.156 (1.30), 8.161 (1.40), 8.514 (16.00), 8.537 (2.78), 8.552 (1.35), 8.624 (3.04), 8.634 (3.04), 10.653 (1.07).

### Example 61

### rac-N-[(4-(oxan-4-yl)-2,5-dioxoimidazolidin-4-yl)methyl]-2-phenyl-2H-1,2,3-triazole-4-carboxamide

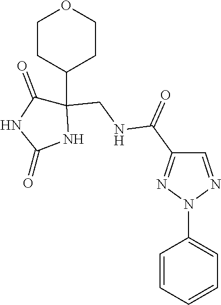 [SMILES: O=C1NC(=O)C(CNC(=O)c2cnn(-c3ccccc3)n2)(C2CCOCC2)N1]

To a solution of rac-5-(aminomethyl)-5-(oxan-4-yl)imidazolidine-2,4-dione hydrochloride (264 mg, 1.06 mmol) and 2-phenyl-2H-1,2,3-triazole-4-carboxylic acid (200 mg, 1.06 mmol) in N,N-dimethylformamide (5.43 ml) was added 1-ethyl-3-(3-dimethylaminopropyl)carbodiimide hydrochloride (263 mg, 1.37 mmol), 1-hydroxybenzotriazole hydrate (210 mg, 1.37 mmol) and N,N-diisopropylethylamine (520 μl, 2.96 mmol). The reaction mixture was stirred at RT for 16 h. Water was added in to the mixture and the resulting precipitate was filtered off, washed with water and dried in vacuo. The obtained amount of product was 327 mg (100% purity, 80% of theory).

LC-MS (Method 7): Rₜ=1.16 min; MS (ESIpos): m/z=385 [M+H]⁺

¹H-NMR (400 MHz, DMSO-d6) δ [ppm]: 1.324 (2.26), 1.353 (4.79), 1.372 (2.96), 1.384 (2.30), 1.405 (1.90), 1.424 (2.16), 1.435 (2.26), 1.455 (1.90), 1.465 (1.83), 1.485 (0.73), 1.497 (0.63), 1.654 (2.69), 1.685 (2.26), 1.922 (1.56), 1.951 (2.79), 1.981 (1.26), 2.328 (0.47), 2.366 (0.53), 2.670 (0.53), 2.710 (0.60), 2.731 (1.70), 2.890 (2.10), 3.222 (2.36), 3.255 (4.69), 3.275 (2.59), 3.287 (2.89), 3.589 (1.66), 3.605 (1.90), 3.623 (4.46), 3.639 (4.16), 3.659 (4.19), 3.674 (4.42), 3.693 (1.90), 3.709 (1.73), 3.833 (2.49), 3.841 (2.69), 3.868 (2.49), 3.885 (2.73), 3.893 (2.73), 3.914 (2.46), 7.487 (2.16), 7.506 (5.92), 7.524 (4.02), 7.606 (7.28), 7.626 (11.04), 7.645 (5.79), 7.896 (8.81), 8.056 (10.41), 8.076 (10.25), 8.379 (2.43), 8.395 (5.19), 8.410 (2.49), 8.465 (16.00), 10.714 (7.62).

### Example 62

### rac-N-[(2,5-dioxo-4-(propan-2-yl)imidazolidin-4-yl)methyl]-2-(4-fluorophenyl)-2H-1,2,3-triazole-4-carboxamide

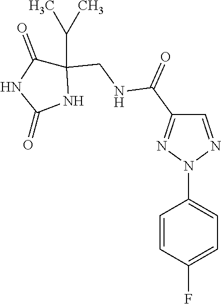 [SMILES: CC(C)C1(CNC(=O)c2cnn(-c3ccc(F)cc3)n2)NC(=O)NC1=O]

To a solution of rac-5-(aminomethyl)-5-(propan-2-yl)imidazolidine-2,4-dione hydrochloride (70.0 mg, 409 μmol) and 2-(4-fluorophenyl)-2H-1,2,3-triazole-4-carboxylic acid (84.7 mg, 409 μmol) in N,N-dimethylformamide (2.10 ml) was added 1-ethyl-3-(3-dimethylaminopropyl)carbodiimide hydrochloride (102 mg, 532 μmol), 1-hydroxybenzotriazole hydrate (81.4 mg, 532 μmol) and N,N-diisopropylethylamine (200 μl, 1.14 mmol). The reaction mixture was stirred at RT for 16 h. Water was added in to the mixture and the resulting precipitate was filtered off, washed with water and dried in vacuo. The obtained amount of product was 53 mg (98% purity, 35% of theory).

LC-MS (Method 7): Rₜ=1.35 min; MS (ESIpos): m/z=361 [M+H]⁺

¹H-NMR (400 MHz, DMSO-d₆) δ[ppm]: 0.842 (15.96), 0.859 (16.00), 0.951 (15.76), 0.968 (15.92), 1.967 (1.03), 1.984 (2.48), 2.001 (3.24), 2.018 (2.26), 2.035 (0.82), 2.328 (0.65), 2.367 (0.46), 2.670 (0.69), 2.710 (0.49), 2.731 (0.63), 2.891 (0.78), 3.585 (1.19), 3.602 (1.41), 3.619 (4.25), 3.637 (5.06), 3.656 (4.06), 3.675 (1.24), 3.691 (1.08), 7.458 (5.67), 7.464 (2.23), 7.480 (10.01), 7.502 (5.86), 7.511 (0.64), 7.804 (8.44), 8.073 (6.02), 8.084 (6.24), 8.090 (3.83), 8.095 (5.94), 8.107 (5.39), 8.323 (2.09), 8.339 (4.05), 8.354 (1.90), 8.464 (13.50), 10.654 (5.41).

### Example 63

### rac-N-[(4-tert-butyl-2,5-dioxoimidazolidin-4-yl)methyl]-2-(4-fluorophenyl)-2H-1,2,3-triazole-4-carboxamide

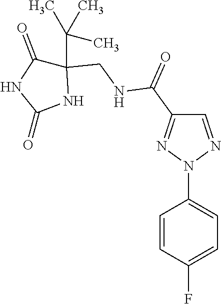 [SMILES: CC(C)(C)C1(CNC(=O)c2cnn(-c3ccc(F)cc3)n2)NC(=O)NC1=O]

To a solution of rac-5-(aminomethyl)-5-tert-butylimidazolidine-2,4-dione (70.0 mg, 378 μmol) and 2-(4-fluorophenyl)-2H-1,2,3-triazole-4-carboxylic acid (78.3 mg, 378 μmol) in N,N-dimethylformamide (1.9 ml) was added 1-ethyl-3-(3-dimethylaminopropyl)carbodiimide hydrochloride (94.2 mg, 491 μmol), 1-hydroxybenzotriazole hydrate (75.2 mg, 491 μmol) and N,N-diisopropylethylamine (180 μl, 1.05 mmol). The reaction mixture was stirred at RT for 16 h. Water was added in to the mixture and the resulting precipitate was filtered off, washed with water and dried in vacuo. The obtained amount of product was 14 mg (100% purity, 10% of theory).

LC-MS (Method 7): Rₜ=1.44 min; MS (ESIpos): m/z=375 [M+H]⁺

¹H-NMR (400 MHz, DMSO-d6) δ [ppm]: 1.018 (16.00), 3.690 (0.62), 3.705 (0.57), 3.759 (0.60), 3.776 (0.64), 7.462 (1.07), 7.484 (1.93), 7.506 (1.11), 7.599 (1.62), 8.061 (1.18), 8.073 (1.24), 8.078 (0.79), 8.084 (1.17), 8.096 (1.05), 8.285 (0.40), 8.302 (0.75), 8.447 (2.82), 10.594 (0.92).

### Example 64

### rac-N-[4-(cyclopropylmethyl)-2,5-dioxoimidazolidin-4-yl)methyl]-2-(4-fluorophenyl)-2H-1,2,3-triazole-4-carboxamide

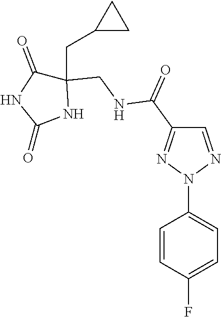 [SMILES: O=C1NC(=O)C(CNC(=O)c2cnn(-c3ccc(F)cc3)n2)(CC2CC2)N1]

To a solution of rac-5-(aminomethyl)-5-(cyclopropylmethyl)imidazolidine-2,4-dione hydrochloride (70.0 mg, 319 μmol) and 2-(4-fluorophenyl)-2H-1,2,3-triazole-4-carboxylic acid (66.0 mg, 319 μmol) in N,N-dimethylformamide (1.6 ml) was added 1-ethyl-3-(3-dimethylaminopropyl)carbodiimide hydrochloride (79.4 mg, 414 μmol), 1-hydroxybenzotriazole hydrate (63.4 mg, 414 μmol) and N,N-diisopropylethylamine (160 μl, 892 μmol). The reaction mixture was stirred at RT for 16 h. Water was added in to the mixture and the resulting precipitate was filtered off, washed with water and dried in vacuo. The obtained amount of product was 43 mg (98% purity, 36% of theory).

LC-MS (Method 7): Rₜ=1.42 min; MS (ESIpos): m/z=373 [M+H]⁺

¹H-NMR (400 MHz, DMSO-d6) δ [ppm]: 0.008 (1.19), 1.663 (0.58), 1.676 (0.85), 1.698 (1.78), 1.710 (1.79), 1.726 (2.48), 1.739 (3.51), 1.751 (2.35), 1.766 (1.95), 1.778 (2.41), 1.800 (0.93), 1.812 (1.95), 1.828 (1.05), 1.844 (1.31), 1.861 (1.57), 1.876 (1.36), 1.890 (0.94), 2.025 (1.19), 2.052 (1.40), 2.087 (0.74), 2.328 (0.69), 2.367 (0.45), 2.670 (0.78), 2.710 (0.53), 2.731 (0.94), 2.891 (1.17), 3.508 (1.32), 3.524 (1.59), 3.542 (4.28), 3.558 (4.19), 3.567 (4.17), 3.584 (4.09), 3.602 (1.42), 3.618 (1.28), 4.938 (3.57), 4.963 (3.72), 4.995 (3.34), 5.000 (3.21), 5.038 (30.72), 5.042 (3.50), 5.727 (0.94), 5.742 (1.99), 5.752 (1.11), 5.758 (1.11), 5.768 (2.56), 5.785 (2.42), 5.795 (0.97), 5.801 (0.99), 5.811 (1.64), 5.827 (0.71), 7.460 (6.15), 7.466 (2.43), 7.483 (10.76), 7.499 (2.39), 7.504 (6.47), 7.811 (8.95), 8.085 (6.55), 8.096 (6.86), 8.102 (4.17), 8.107 (6.55), 8.119 (6.01), 8.475 (16.00), 8.541 (2.17), 8.557 (4.35), 8.572 (2.05), 10.713 (4.61).

### Example 65

### rac-N-[(2,5-dioxo-4-propylimidazolidin-4-yl)methyl]-2-(4-fluorophenyl)-2H-1,2,3-triazole-4-carboxamide

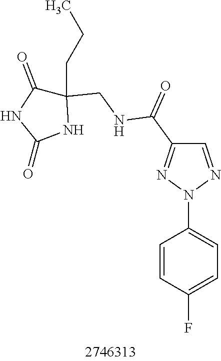 [SMILES: CCCC1(CNC(=O)c2cnn(-c3ccc(F)cc3)n2)NC(=O)NC1=O]

To a solution of rac-5-(aminomethyl)-5-propylimidazolidine-2,4-dione hydrochloride (70.0 mg, 337 μmol) and 2-(4-fluorophenyl)-2H-1,2,3-triazole-4-carboxylic acid (69.8 mg, 337 μmol) in

N,N-dimethylformamide (1.7 ml) was added 1-ethyl-3-(3-dimethylaminopropyl)carbodiimide hydrochloride (84.0 mg, 438 μmol), 1-hydroxybenzotriazole hydrate (67.1 mg, 438 μmol) and N,N-diisopropylethylamine (160 μl, 944 μmol). The reaction mixture was stirred at RT for 16 h. Water was added in to the mixture and the resulting precipitate was filtered off, washed with water and dried in vacuo. The obtained amount of product was 45 mg (100% purity, 36% of theory).

LC-MS (Method 7): Rₜ=1.38 min; MS (ESIpos): m/z=361 [M+H]⁺

¹H-NMR (400 MHz, DMSO-d6) δ [ppm]: 0.844 (6.75), 0.863 (16.00), 0.881 (8.10), 1.052 (0.48), 1.066 (0.89), 1.083 (1.36), 1.098 (1.41), 1.113 (1.38), 1.130 (0.89), 1.145 (0.56), 1.264 (0.62), 1.277 (0.96), 1.294 (1.40), 1.308 (1.30), 1.325 (1.38), 1.337 (0.73), 1.343 (0.78), 1.355 (0.46), 1.540 (0.80), 1.552 (0.97), 1.574 (2.06), 1.586 (1.84), 1.603 (2.08), 1.615 (3.09), 1.628 (2.04), 1.644 (1.77), 1.656 (1.67), 1.678 (0.81), 1.691 (0.55), 3.488 (1.31), 3.503 (1.52), 3.522 (3.84), 3.538 (3.63), 3.551 (3.60), 3.567 (3.66), 3.585 (1.37), 3.602 (1.23), 7.460 (5.03), 7.482 (9.13), 7.504 (5.21), 7.784 (8.05), 8.084 (5.41), 8.096 (5.59), 8.101 (3.45), 8.107 (5.28), 8.118 (4.83), 8.473 (11.89), 8.500 (2.01), 8.516 (3.89), 8.532 (1.81), 10.679 (5.50).

### Example 66

### rac-N-[(4-cyclopentyl-2,5-dioxoimidazolidin-4-yl]methyl]-2-(4-fluorophenyl)-2H-1,2,3-triazole-4-carboxamide

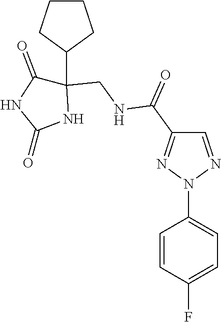 [SMILES: O=C1NC(=O)C(CNC(=O)c2cnn(-c3ccc(F)cc3)n2)(C2CCCC2)N1]

To a solution of rac-5-(aminomethyl)-5-cyclopentylimidazolidine-2,4-dione hydrochloride (100 mg, 507 μmol) and 2-(4-fluorophenyl)-2H-1,2,3-triazole-4-carboxylic acid (105 mg, 507 μmol) in N,N-dimethylformamide (2.6 ml) was added 1-ethyl-3-(3-dimethylaminopropyl)carbodiimide hydrochloride (126 mg, 659 μmol), 1-hydroxybenzotriazole hydrate (101 mg, 659 μmol) and N,N-diisopropylethylamine (250 μl, 1.420 mmol). The reaction mixture was stirred at RT for 16 h. Water was added in to the mixture and the resulting precipitate was filtered off, washed with water and dried in vacuo. The obtained amount of product was 102 mg (96% purity, 50% of theory).

LC-MS (Method 7): Rₜ=1.51 min; MS (ESIpos): m/z=387 [M+H]⁺

¹H-NMR (400 MHz, DMSO-d6) δ [ppm]: −0.149 (0.40), −0.008 (3.37), 0.008 (4.08), 0.937 (0.52), 0.953 (0.53), 1.230 (1.42), 1.274 (0.56), 1.332 (0.98), 1.354 (1.42), 1.370 (1.65), 1.401 (0.73), 1.469 (2.88), 1.488 (4.63), 1.502 (4.62), 1.530 (3.62), 1.546 (3.19), 1.555 (3.01), 1.566 (2.67), 1.767 (1.59), 2.167 (0.57), 2.189 (1.49), 2.210 (1.99), 2.230 (1.33), 2.328 (0.40), 2.366 (0.52), 2.670 (0.46), 2.710 (0.55), 2.731 (0.60), 2.891 (0.77), 3.571 (0.42), 3.587 (0.59), 3.605 (5.73), 3.609 (5.77), 3.624 (6.26), 3.644 (0.65), 3.660 (0.44), 7.458 (5.85), 7.464 (2.25), 7.480 (10.02), 7.497 (2.22), 7.502 (6.40), 7.511 (0.79), 7.839 (8.55), 8.063 (0.70), 8.072 (6.03), 8.084 (6.47), 8.089 (3.96), 8.095 (6.39), 8.101 (2.53), 8.107 (5.88), 8.388 (1.95), 8.405 (4.12), 8.420 (1.97), 8.462 (16.00), 10.648 (3.85).

### Example 67

### rac-2-(4-fluorophenyl)-N-[4-(oxan-4-yl)-2,5-dioxoimidazolidin-4-yl)methyl]-2H-1,2,3-triazole-4-carboxamide

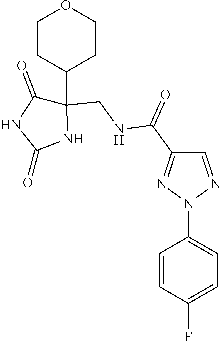 [SMILES: O=C1NC(=O)C(CNC(=O)c2cnn(-c3ccc(F)cc3)n2)(C2CCOCC2)N1]

To a solution of rac-5-(aminomethyl)-5-(oxan-4-yl)imidazolidine-2,4-dione hydrochloride (183 mg, 733 μmol) and 2-(4-fluorophenyl)-2H-1,2,3-triazole-4-carboxylic acid (152 mg, 733 μmol) in N,N-dimethylformamide (3.8 ml) was added 1-ethyl-3-(3-dimethylaminopropyl)carbodiimide hydrochloride (183 mg, 953 μmol), 1-hydroxybenzotriazole hydrate (146 mg, 953 μmol) and N,N-diisopropylethylamine (360 μl, 2.05 mmol). The reaction mixture was stirred at RT for 16 h. Water was added into the mixture and the resulting precipitate was filtered off, washed with water and dried in vacuo. The obtained amount of product was 32 mg (100% purity, 11% of theory).

LC-MS (Method 7): Rₜ=1.21 min; MS (ESIpos): m/z=403 [M+H]⁺

¹H-NMR (400 MHz, DMSO-d6) δ [ppm]: 1.235 (0.60), 1.323 (2.31), 1.353 (4.77), 1.373 (2.44), 1.384 (2.23), 1.404 (1.81), 1.422 (2.15), 1.433 (2.20), 1.452 (1.89), 1.464 (1.84), 1.484 (0.81), 1.651 (2.60), 1.682 (2.20), 1.919 (1.50), 1.949 (2.60), 1.978 (1.21), 2.328 (0.55), 2.367 (0.60), 2.670 (0.55), 2.710 (0.60), 2.731 (1.00), 2.891 (1.21), 3.231 (2.44), 3.253 (4.72), 3.580 (1.65), 3.596 (1.84), 3.614 (3.88), 3.631 (3.62), 3.658 (3.67), 3.674 (3.88), 3.692 (1.86), 3.708 (1.68), 3.840 (2.60), 3.869 (2.41), 3.884 (2.62), 3.894 (2.60), 3.913 (2.36), 7.460 (6.22), 7.482 (11.38), 7.503 (6.66), 7.890 (9.26), 8.075 (6.77), 8.087 (7.29), 8.092 (5.01), 8.098 (7.06), 8.110 (6.37), 8.391 (2.39), 8.406 (4.77), 8.422 (2.31), 8.466 (16.00), 10.710 (5.69).

### Example 68

### rac-N-[(4-butyl-2,5-dioxoimidazolidin-4-yl)methyl]-2-(4-fluorophenyl)-2H-1,2,3-triazole-4-carboxamide

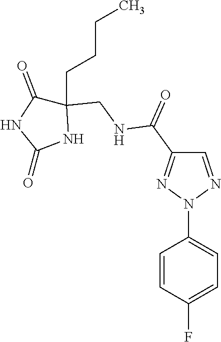 [SMILES: CCCCC1(CNC(=O)c2cnn(-c3ccc(F)cc3)n2)NC(=O)NC1=O]

To a solution of rac-5-(aminomethyl)-5-butylimidazolidine-2,4-dione (200.0 mg, 1.08 mmol) and 2-(4-fluorophenyl)-2H-1,2,3-triazole-4-carboxylic acid (223.7 mg, 1.080 mmol) in N,N-dimethylformamide (5.5 ml) was added 1-ethyl-3-(3-dimethylaminopropyl)carbodiimide hydrochloride (269.1 mg, 1.40 mmol), 1-hydroxybenzotriazole hydrate (215.0 mg, 1.40 mmol) and N,N-diisopropylethylamine (527 μl, 3.02 mmol). The reaction mixture was stirred at RT for 16 h. Water was added in to the mixture and the resulting precipitate was filtered of f, washed with water and dried in vacuo. The obtained amount of product was 50 mg (100% purity, 12% of theory).

LC-MS (Method 7): Rₜ=1.49 min; MS (ESIpos): m/z=375 [M+H]⁺

¹H-NMR (400 MHz, DMSO-d6) δ [ppm]: −0.149 (0.49), −0.008 (5.01), 0.008 (4.65), 0.146 (0.49), 0.832 (6.56), 0.850 (16.00), 0.867 (8.38), 1.007 (0.53), 1.021 (1.02), 1.038 (1.60), 1.050 (1.51), 1.068 (1.33), 1.213 (0.89), 1.232 (2.70), 1.249 (5.14), 1.262 (6.12), 1.274 (5.27), 1.290 (2.08), 1.557 (0.80), 1.567 (1.02), 1.592 (2.26), 1.620 (1.95), 1.631 (1.77), 1.639 (1.60), 1.652 (1.51), 1.667 (1.99), 1.701 (0.93), 1.714 (0.53), 2.367 (0.49), 2.670 (0.40), 2.710 (0.49), 3.495 (1.15), 3.511 (1.37), 3.529 (4.48), 3.547 (6.60), 3.564 (4.39), 3.582 (1.33), 3.598 (1.20), 7.459 (6.03), 7.465 (2.35), 7.481 (10.50), 7.498 (2.39), 7.503 (6.65), 7.512 (0.80), 7.774 (8.02), 8.074 (0.75), 8.083 (6.29), 8.088 (2.88), 8.094 (6.69), 8.100 (4.21), 8.105 (6.60), 8.112 (2.66), 8.117 (6.12), 8.472 (15.96), 8.502 (2.13), 8.518 (4.39), 8.534 (2.08), 10.674 (5.81).

### Example 69

### rac-N-[(4-cyclobutyl-2,5-dioxoimidazolidin-4-yl)methyl]-2-(4-fluorophenyl)-2H-1,2,3-triazole-4-carboxamide

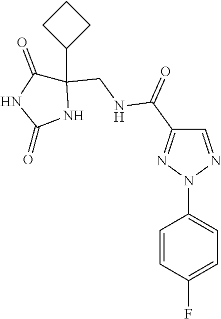 [SMILES: O=C1NC(=O)C(CNC(=O)c2cnn(-c3ccc(F)cc3)n2)(C2CCC2)N1]

To a solution of rac-5-(aminomethyl)-5-cyclobutylimidazolidine-2,4-dione hydrochloride (100 mg, 546 μmol) and 2-(4-fluorophenyl)-2H-1,2,3-triazole-4-carboxylic acid (113 mg, 546 μmol) in N,N-dimethylformamide (2.80 ml) was added 1-ethyl-3-(3-dimethylaminopropyl)carbodiimide hydrochloride (136 mg, 710 μmol), 1-hydroxybenzotriazole hydrate (109 mg, 710 μmol) and N,N-diisopropylethylamine (266 μl, 1.58 mmol). The reaction mixture was stirred at RT for 16 h. Water was added in to the mixture and the resulting precipitate was filtered off, washed with water and dried in vacuo. The obtained amount of product was 104 mg (98% purity, 50% of theory).

LC-MS (Method 7): Rₜ=1.43 min; MS (ESIpos): m/z=373 [M+H]⁺

¹H-NMR (400 MHz, DMSO-d6) δ [ppm]: −0.008 (1.97), 0.008 (2.47), 1.668 (1.58), 1.710 (1.43), 1.724 (1.64), 1.730 (1.70), 1.746 (1.45), 1.767 (2.31), 1.789 (2.67), 1.809 (2.60), 1.821 (1.54), 1.830 (1.64), 1.851 (1.00), 1.872 (0.62), 1.904 (2.09), 1.927 (2.96), 1.939 (1.72), 1.950 (2.12), 1.972 (0.76), 2.674 (0.64), 2.696 (1.53), 2.717 (2.37), 2.732 (1.64), 2.738 (1.53), 2.759 (0.45), 2.891 (1.12), 3.445 (1.15), 3.460 (1.35), 3.479 (3.79), 3.495 (3.69), 3.504 (3.74), 3.520 (3.77), 3.538 (1.31), 3.555 (1.20), 7.452 (0.56), 7.461 (5.45), 7.466 (2.05), 7.483 (9.43), 7.499 (2.01), 7.505 (6.00), 7.513 (0.69), 7.983 (7.71), 8.066 (0.63), 8.075 (5.73), 8.080 (2.47), 8.087 (6.08), 8.092 (3.67), 8.098 (6.01), 8.104 (2.31), 8.110 (5.56), 8.399 (1.82), 8.414 (3.77), 8.430 (1.79), 8.461 (16.00), 8.476 (0.46), 10.657 (4.43).

### Example 70

### ent-N-[(2,5-dioxo-4-(propan-2-yl)imidazolidin-4-yl)methyl]-2-phenyl-2H-1,2,3-triazole-4-carboxamide

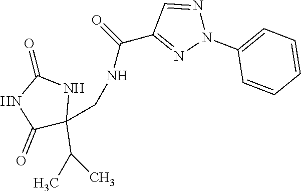 [SMILES: CC(C)C1(CNC(=O)c2cnn(-c3ccccc3)n2)NC(=O)NC1=O]

Enantiomeric separation of rac-N-[(2,5-dioxo-4-(propan-2-yl)imidazolidin-4-yl)methyl]-2-phenyl-2H-1,2,3-triazole-4-carboxamide (149 mg) using method 1D afforded 65 mg of desired product.

LC-MS (Method 7): Rₜ=1.25 min; MS (ESIpos): m/z=343 [M+H]⁺

HPLC (Methode 1E): Rₜ=6.28 min, >99.0% ee

¹H-NMR (400 MHz, DMSO-d6) δ[ppm]: −0.008 (1.50), 0.008 (1.29), 0.845 (14.14), 0.862 (14.54), 0.953 (13.80), 0.970 (14.25), 1.236 (0.40), 1.971 (0.81), 1.988 (2.13), 2.005 (2.81), 2.022 (1.98), 2.038 (0.71), 3.595 (0.87), 3.611 (0.97), 3.629 (3.91), 3.644 (5.92), 3.658 (3.85), 3.677 (0.86), 3.692 (0.76), 7.487 (1.79), 7.505 (4.73), 7.524 (3.18), 7.605 (5.78), 7.626 (8.26), 7.645 (4.38), 7.810 (5.60), 8.055 (8.10), 8.074 (7.99), 8.077 (5.76), 8.312 (1.66), 8.328 (3.47), 8.343 (1.60), 8.464 (16.00), 10.660 (4.84).

### Example 71

### ent-N-[(4-tert-butyl-2,5-dioxoimidazolidin-4-yl)methyl]-2-phenyl-2H-1,2,3-triazole-4-carboxamide

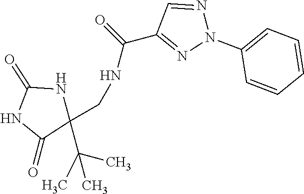 [SMILES: CC(C)(C)C1(CNC(=O)c2cnn(-c3ccccc3)n2)NC(=O)NC1=O]

Enantiomeric separation of rac-N-[(4-tert-butyl-2,5-dioxoimidazolidin-4-yl)methyl]-2-phenyl-2H-1,2,3-triazole-4-carboxamide (26 mg) using method 2D afforded 9 mg of desired product.

LC-MS (Method 7): Rₜ=1.39 min; MS (ESIpos): m/z=357 [M+H]⁺

HPLC (Methode 2E): Rₜ=4.54 min, >99.0% ee

¹H-NMR (400 MHz, DMSO-d6) δ [ppm]: −0.008 (0.41), 1.020 (16.00), 3.688 (0.59), 3.703 (0.54), 3.772 (0.54), 3.789 (0.59), 7.506 (0.94), 7.524 (0.63), 7.606 (2.35), 7.626 (1.70), 7.645 (0.90), 8.044 (1.57), 8.063 (1.58), 8.066 (1.13), 8.288 (0.63), 8.447 (2.77), 10.590 (0.44).

### Example 72

### ent-N-[(4-(cyclopropylmethyl)-2,5-dioxoimidazolidin-4-yl]methyl]-2-(4-fluorophenyl)-2H-1,2,3-triazole-4-carboxamide

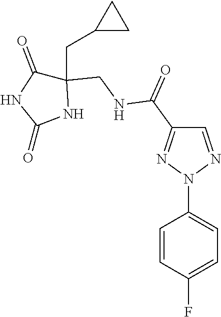 [SMILES: O=C1NC(=O)C(CNC(=O)c2cnn(-c3ccc(F)cc3)n2)(CC2CC2)N1]

Enantiomeric separation of rac-N-[(4-(cyclopropylmethyl)-2,5-dioxoimidazolidin-4-yl]methyl]-2-(4-fluorophenyl)-2H-1,2,3-triazole-4-carboxamide (38 mg) using method 4D afforded 10 mg of desired product.

LC-MS (Method 8): Rₜ=0.77 min; MS (ESIpos): m/z=373 [M+H]⁺

HPLC (Methode 4E): Rₜ=2.63 min, >99.0% ee

¹H-NMR (400 MHz, DMSO-d6) δ [ppm]: −0.149 (1.03), −0.008 (9.10), 0.008 (8.84), 0.146 (0.96), 0.860 (0.63), 1.101 (1.29), 1.109 (0.94), 1.234 (1.47), 1.711 (1.22), 1.725 (1.68), 1.739 (2.46), 1.751 (1.64), 1.778 (1.68), 1.812 (1.36), 1.863 (1.15), 2.052 (1.01), 2.160 (0.42), 2.328 (0.58), 2.367 (0.65), 2.670 (0.70), 2.710 (0.84), 3.508 (1.05), 3.524 (1.01), 3.542 (2.97), 3.558 (2.81), 3.567 (3.11), 3.583 (2.85), 3.601 (1.03), 3.618 (0.98), 3.722 (0.80), 3.740 (0.73), 4.033 (2.01), 4.938 (2.67), 4.963 (2.78), 4.995 (2.48), 5.038 (2.90), 5.742 (1.54), 5.768 (1.99), 5.785 (1.89), 5.811 (1.31), 5.826 (0.56), 7.460 (4.65), 7.482 (7.77), 7.504 (5.05), 7.813 (4.77), 8.085 (4.80), 8.096 (5.31), 8.102 (3.11), 8.107 (5.03), 8.119 (4.82), 8.474 (16.00), 8.541 (1.40), 8.557 (3.09), 8.572 (1.45), 10.714 (4.33).

### Example 73

### ent-N-[(2,5-dioxo-4-propylimidazolidin-4-yl)methyl]-2-(4-fluorophenyl)-2H-1,2,3-triazole-4-carboxamide

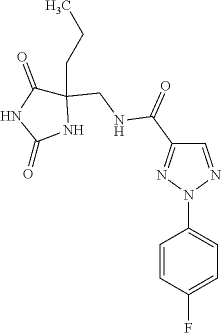 [SMILES: CCCC1(CNC(=O)c2cnn(-c3ccc(F)cc3)n2)NC(=O)NC1=O]

Enantiomeric separation of rac-N-[(2,5-dioxo-4-propylimidazolidin-4-yl)methyl]-2-(4-fluorophenyl)-2H-1,2,3-triazole-4-carboxamide (39 mg) using method 5D afforded 20 mg of desired product.

LC-MS (Method 7): Rₜ=1.36 min; MS (ESIpos): m/z=361 [M+H]⁺

HPLC (Methode 5E): Rₜ=2.21 min, >99.0% ee

¹H-NMR (400 MHz, DMSO-d6) δ [ppm]: −0.149 (0.40), −0.008 (4.24), 0.008 (3.87), 0.844 (6.50), 0.862 (16.00), 0.881 (8.05), 1.035 (0.48), 1.052 (0.72), 1.065 (1.09), 1.083 (2.10), 1.101 (2.64), 1.113 (1.80), 1.119 (1.82), 1.127 (1.51), 1.144 (1.30), 1.171 (1.27), 1.235 (2.20), 1.259 (1.13), 1.277 (1.29), 1.295 (1.63), 1.306 (1.40), 1.325 (1.46), 1.337 (0.84), 1.343 (0.87), 1.355 (0.59), 1.540 (0.74), 1.552 (0.89), 1.575 (1.87), 1.586 (1.69), 1.603 (1.87), 1.615 (2.87), 1.628 (1.94), 1.644 (1.65), 1.656 (1.62), 1.678 (0.81), 1.691 (0.57), 3.488 (1.24), 3.504 (1.44), 3.522 (3.58), 3.538 (3.39), 3.551 (3.39), 3.567 (3.49), 3.585 (1.39), 3.601 (1.30), 3.705 (0.50), 3.723 (0.94), 3.741 (1.06), 3.759 (0.87), 3.839 (1.77), 7.460 (5.12), 7.465 (1.94), 7.482 (8.80), 7.498 (2.01), 7.504 (5.56), 7.512 (0.64), 7.786 (5.77), 8.084 (5.32), 8.089 (2.44), 8.096 (5.66), 8.101 (3.48), 8.107 (5.54), 8.113 (2.20), 8.119 (5.08), 8.473 (14.35), 8.500 (1.78), 8.516 (3.64), 8.532 (1.72), 10.679 (4.99).

### Example 74

### ent-N-[(4-cyclopentyl-2,5-dioxoimidazolidin-4-yl)methyl]-2-(4-fluorophenyl)-2H-1,2,3-triazole-4-carboxamide

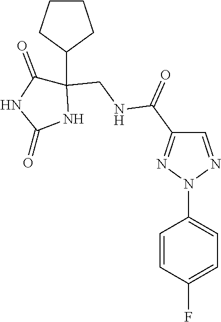 [SMILES: O=C1NC(=O)C(CNC(=O)c2cnn(-c3ccc(F)cc3)n2)(C2CCCC2)N1]

Enantiomeric separation of rac-N-[(4-cyclopentyl-2,5-dioxoimidazolidin-4-yl)methyl]-2-(4-fluorophenyl)-2H-1,2,3-triazole-4-carboxamide (93 mg) using method 6D afforded 33 mg of desired product.

LC-MS (Method 7): Rₜ=1.51 min; MS (ESIpos): m/z=387 [M+H]⁺

HPLC (Methode 6E): Rₜ=3.74 min, >99.0% ee

¹H-NMR (400 MHz, DMSO-d6) δ [ppm]: −0.008 (3.04), 0.008 (3.05), 1.134 (1.32), 1.152 (2.79), 1.170 (1.56), 1.235 (1.94), 1.273 (0.62), 1.310 (0.47), 1.356 (1.43), 1.374 (1.24), 1.400 (0.67), 1.469 (2.82), 1.488 (4.56), 1.502 (4.51), 1.530 (3.52), 1.546 (3.10), 1.555 (2.92), 1.567 (2.54), 1.767 (1.58), 1.784 (1.33), 2.168 (0.57), 2.189 (1.47), 2.210 (1.96), 2.230 (1.31), 2.903 (1.00), 2.921 (0.97), 3.571 (0.41), 3.588 (0.57), 3.606 (5.80), 3.610 (5.78), 3.625 (6.23), 3.644 (0.59), 3.660 (0.41), 7.458 (5.63), 7.464 (2.09), 7.480 (10.15), 7.497 (2.17), 7.502 (6.15), 7.511 (0.71), 7.841 (9.12), 8.072 (6.04), 8.084 (6.39), 8.089 (3.86), 8.095 (6.21), 8.102 (2.42), 8.107 (5.78), 8.389 (1.94), 8.405 (4.02), 8.421 (1.91), 8.462 (16.00).

### Example 75

### ent-N-[(4-cyclohexyl-2,5-dioxoimidazolidin-4-yl)methyl]-2-(4-fluorophenyl)-2H-1,2,3-triazole-4-carboxamide

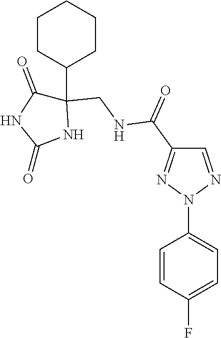 [SMILES: O=C1NC(=O)C(CNC(=O)c2cnn(-c3ccc(F)cc3)n2)(C2CCCCC2)N1]

Enantiomeric separation of rac-N-[(4-cyclohexyl-2,5-dioxoimidazolidin-4-yl)methyl]-2-(4-fluorophenyl)-2H-1,2,3-triazole-4-carboxamide (98 mg) using method 7D afforded 20 mg of desired product.

LC-MS (Method 8): Rₜ=0.89 min; MS (ESIpos): m/z=401 [M+H]⁺

HPLC (Methode 7E): Rₜ=3.28 min, >99.0% ee

¹H-NMR (400 MHz, DMSO-d6) δ [ppm]: −0.008 (3.63), 0.008 (2.78), 0.844 (0.55), 0.857 (0.67), 1.011 (0.90), 1.042 (2.33), 1.072 (2.97), 1.101 (2.47), 1.119 (2.21), 1.141 (5.16), 1.159 (5.83), 1.169 (4.23), 1.176 (4.46), 1.193 (1.87), 1.235 (2.43), 1.484 (1.66), 1.507 (1.87), 1.596 (1.78), 1.627 (2.01), 1.688 (2.66), 1.727 (2.13), 1.760 (1.49), 1.803 (1.63), 1.833 (1.53), 2.328 (0.43), 2.367 (0.46), 2.919 (1.06), 2.937 (1.05), 3.580 (1.25), 3.596 (1.45), 3.614 (3.28), 3.630 (3.04), 3.648 (3.05), 3.663 (3.20), 3.682 (1.34), 3.698 (1.17), 7.458 (5.40), 7.463 (1.99), 7.480 (9.38), 7.496 (2.14), 7.502 (5.88), 7.510 (0.76), 7.785 (7.88), 8.063 (0.67), 8.071 (5.72), 8.077 (2.46), 8.083 (6.07), 8.089 (3.67), 8.094 (5.96), 8.101 (2.35), 8.106 (5.50), 8.300 (1.79), 8.316 (3.67), 8.332 (1.79), 8.462 (16.00), 10.638 (1.93).

### Example 76

### ent-N-[(2,5-dioxo-4-(propan-2-yl)imidazolidin-4-yl)methyl]-2-(4-fluorophenyl)-2H-1,2,3-triazole-4-carboxamide

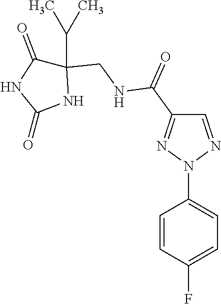 [SMILES: CC(C)C1(CNC(=O)c2cnn(-c3ccc(F)cc3)n2)NC(=O)NC1=O]

Enantiomeric separation of rac-N-[(2,5-dioxo-4-(propan-2-yl)imidazolidin-4-yl)methyl]-2-(4-fluorophenyl)-2H-1,2,3-triazole-4-carboxamide (48 mg) using method 8D afforded 19 mg of desired product.

HPLC (Methode 8E): Rₜ=2.01 min, >95.0% ee

### Example 77

### ent-N-[(4-cyclobutyl-2,5-dioxoimidazolidin-4-yl)methyl]-2-(4-fluorophenyl)-2H-1,2,3-triazole-4-carboxamide

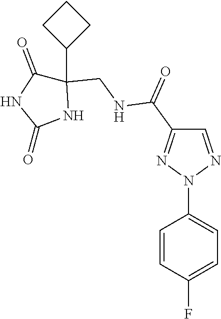 [SMILES: O=C1NC(=O)C(CNC(=O)c2cnn(-c3ccc(F)cc3)n2)(C2CCC2)N1]

Enantiomeric separation of rac-N-[(4-cyclobutyl-2,5-dioxoimidazolidin-4-yl)methyl]-2-(4-fluorophenyl)-2H-1,2,3-triazole-4-carboxamide (98 mg) using method 9D afforded 34 mg of desired product.

LC-MS (Method 8): Rₜ=0.80 min; MS (ESIpos): m/z=373 [M+H]⁺

HPLC (Methode 9E): Rₜ=2.60 min, >98.0% ee

¹H-NMR (400 MHz, DMSO-d6) δ[ppm]: −0.008 (2.17), 0.008 (2.54), 1.234 (0.41), 1.668 (1.42), 1.703 (1.27), 1.730 (1.53), 1.745 (1.28), 1.767 (2.07), 1.789 (2.40), 1.809 (2.33), 1.821 (1.36), 1.830 (1.45), 1.850 (0.88), 1.872 (0.53), 1.904 (1.87), 1.928 (2.63), 1.938 (1.53), 1.950 (1.90), 1.971 (0.67), 2.674 (0.68), 2.696 (1.42), 2.717 (2.15), 2.738 (1.37), 3.445 (1.06), 3.460 (1.22), 3.479 (3.47), 3.495 (3.38), 3.504 (3.43), 3.520 (3.46), 3.538 (1.17), 3.555 (1.07), 7.461 (4.96), 7.466 (1.74), 7.483 (8.75), 7.499 (1.84), 7.505 (5.43), 7.983 (7.78), 8.066 (0.56), 8.075 (5.33), 8.080 (2.21), 8.087 (5.64), 8.092 (3.30), 8.098 (5.48), 8.104 (2.09), 8.109 (5.11), 8.399 (1.60), 8.415 (3.44), 8.431 (1.64), 8.461 (16.00), 10.559 (0.56).

### Example 78

### ent-N-[(4-butyl-2,5-dioxoimidazolidin-4-yl)methyl]-2-phenyl-2H-1,2,3-triazole-4-carboxamide

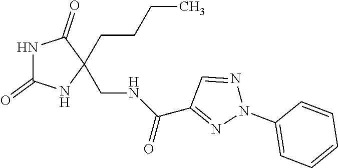 [SMILES: CCCCC1(CNC(=O)c2cnn(-c3ccccc3)n2)NC(=O)NC1=O]

Enantiomeric separation of rac-N-[(4-butyl-2,5-dioxoimidazolidin-4-yl)methyl]-2-phenyl-2H-1,2,3-triazole-4-carboxamide (23 mg) using method 10D afforded 10 mg of desired product.

LC-MS (Method 7): Rₜ=1.45 min; MS (ESIpos): m/z=357 [M+H]⁺

HPLC (Methode 9E): Rₜ=2.49 min, >99.0% ee

¹H-NMR (400 MHz, DMSO-d6) δ [ppm]: −0.008 (1.24), 0.008 (1.17), 0.832 (6.26), 0.850 (15.48), 0.867 (8.03), 1.009 (0.51), 1.022 (0.98), 1.040 (1.49), 1.052 (1.36), 1.069 (1.21), 1.084 (0.66), 1.213 (0.87), 1.232 (2.85), 1.249 (4.84), 1.263 (5.58), 1.274 (4.83), 1.290 (1.87), 1.555 (0.81), 1.565 (0.97), 1.590 (2.12), 1.618 (1.92), 1.629 (1.66), 1.638 (1.45), 1.651 (1.37), 1.666 (1.79), 1.700 (0.83), 1.712 (0.48), 2.524 (1.07), 3.498 (0.91), 3.514 (1.09), 3.532 (4.47), 3.547 (6.81), 3.562 (4.31), 3.579 (1.00), 3.596 (0.88), 7.486 (2.00), 7.504 (5.33), 7.522 (3.59), 7.606 (6.57), 7.627 (9.42), 7.640 (2.03), 7.645 (5.06), 7.745 (4.45), 8.065 (8.85), 8.084 (8.88), 8.087 (6.42), 8.472 (16.00), 8.494 (1.89), 8.510 (3.88), 8.526 (1.81), 10.659 (1.57).

### Example 79

### ent-N-[(4-cyclobutyl-2,5-dioxoimidazolidin-4-yl)methyl]-2-phenyl-2H-1,2,3-triazole-4-carboxamide

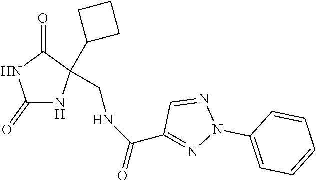 [SMILES: O=C1NC(=O)C(CNC(=O)c2cnn(-c3ccccc3)n2)(C2CCC2)N1]

Enantiomeric separation of rac-N-{[(4R)-4-cyclobutyl-2,5-dioxoimidazolidin-4-yl]methyl}-2-phenyl-2H-1,2,3-triazole-4-carboxamide (126 mg) using method 11 D afforded 55 mg of desired product.

LC-MS (Method 7): Rₜ=1.34 min; MS (ESIpos): m/z=355 [M+H]⁺

HPLC (Methode 10E): Rₜ=5.78 min, >99.0% ee

¹H-NMR (400 MHz, DMSO-d6) δ [ppm]: −0.008 (1.50), 0.008 (1.63), 1.235 (0.55), 1.669 (1.17), 1.711 (1.05), 1.732 (1.27), 1.747 (1.06), 1.769 (1.74), 1.791 (1.97), 1.811 (1.89), 1.832 (1.21), 1.852 (0.74), 1.905 (1.57), 1.929 (2.19), 1.952 (1.59), 1.975 (0.55), 2.676 (0.54), 2.701 (1.17), 2.721 (1.80), 2.742 (1.18), 3.453 (0.83), 3.469 (0.96), 3.487 (3.08), 3.507 (3.75), 3.523 (3.10), 3.541 (0.92), 3.558 (0.86), 7.487 (1.61), 7.506 (4.07), 7.524 (2.85), 7.607 (5.00), 7.628 (6.96), 7.642 (1.52), 7.647 (3.76), 7.990 (6.18), 8.059 (7.00), 8.077 (7.04), 8.080 (5.10), 8.391 (1.35), 8.406 (2.86), 8.422 (1.36), 8.461 (16.00), 10.661 (3.78).

### Example 80

### ent-N-[(4-(cyclopropylmethyl)-2,5-dioxoimidazolidin-4-yl)methyl]-3-phenyl-1,2,4-oxadiazole-5-carboxamide

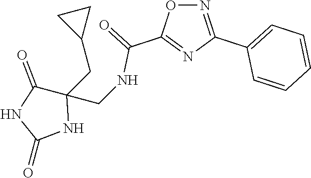 [SMILES: O=C1NC(=O)C(CNC(=O)c2nc(-c3ccccc3)no2)(CC2CC2)N1]

Enantiomeric separation of rac-N-[(4-(cyclopropylmethyl)-2,5-dioxoimidazolidin-4-yl)methyl]-3-phenyl-1,2,4-oxadiazole-5-carboxamide (13 mg) using method 12D afforded 3 mg of desired product.

HPLC (Methode 10E): Rₜ=6.20 min, >99.0% ee

¹H-NMR (400 MHz, DMSO-d6) δ[ppm]: 1.236 (0.58), 1.717 (0.60), 1.736 (2.32), 1.750 (3.62), 1.762 (6.97), 1.778 (5.13), 1.784 (4.55), 1.807 (1.45), 1.824 (1.56), 1.843 (1.70), 1.861 (2.17), 2.004 (0.91), 2.021 (1.82), 2.040 (2.18), 2.057 (1.60), 2.078 (1.06), 2.328 (0.64), 2.366 (0.54), 2.670 (0.82), 2.711 (0.67), 3.482 (3.56), 3.516 (5.95), 3.597 (5.83), 3.631 (3.44), 4.946 (4.87), 4.971 (5.19), 5.006 (4.52), 5.010 (4.50), 5.049 (5.20), 5.053 (5.01), 5.733 (1.30), 5.749 (2.80), 5.759 (1.45), 5.765 (1.48), 5.775 (3.54), 5.792 (3.30), 5.802 (1.29), 5.808 (1.35), 5.818 (2.33), 5.833 (1.05), 7.594 (2.33), 7.600 (3.33), 7.616 (12.07), 7.634 (16.00), 7.654 (4.34), 7.673 (1.21), 7.827 (3.87), 8.055 (10.59), 8.060 (11.07), 8.075 (11.07), 8.079 (9.36), 9.426 (1.53), 10.771 (1.41).

### Example 81

### ent-2-(4-fluorophenyl)-N-[4-(oxan-4-yl)-2,5-dioxoimidazolidin-4-yl)methyl]-2H-1,2,3-triazole-4-carboxamide

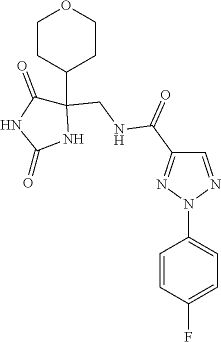 [SMILES: O=C1NC(=O)C(CNC(=O)c2cnn(-c3ccc(F)cc3)n2)(C2CCOCC2)N1]

Enantiomeric separation of ent-2-(4-fluorophenyl)-N-[4-(oxan-4-yl)-2,5-dioxoimidazolidin-4-yl)methyl]-2H-1,2,3-triazole-4-carboxamide (20 mg) using method 15D afforded 7 mg of desired product.

LC-MS (Method 8): Rₜ=0.68 min; MS (ESIpos): m/z=403 [M+H]⁺

HPLC (Methode 14E): Rₜ=2.59 min, >99.0% ee

¹H-NMR (400 MHz, DMSO-d6) δ [ppm]: 0.008 (0.92), 0.854 (0.49), 0.996 (0.40), 1.235 (1.92), 1.322 (1.87), 1.353 (3.69), 1.372 (1.96), 1.384 (1.77), 1.403 (1.46), 1.422 (1.69), 1.433 (1.73), 1.452 (1.49), 1.463 (1.41), 1.483 (0.68), 1.495 (0.60), 1.651 (2.10), 1.679 (1.73), 1.919 (1.22), 1.949 (2.07), 1.978 (1.05), 2.328 (0.51), 2.367 (0.55), 2.670 (0.44), 2.711 (0.46), 3.230 (1.79), 3.251 (3.28), 3.273 (1.98), 3.284 (2.17), 3.580 (1.34), 3.596 (1.51), 3.615 (3.20), 3.631 (2.90), 3.656 (2.92), 3.672 (3.12), 3.691 (1.47), 3.707 (1.34), 3.832 (1.79), 3.840 (1.93), 3.860 (1.67), 3.868 (1.71), 3.884 (1.83), 3.893 (1.86), 3.913 (1.64), 3.921 (1.53), 7.460 (5.55), 7.465 (1.99), 7.482 (9.40), 7.498 (2.12), 7.504 (5.90), 7.890 (7.70), 8.075 (5.81), 8.081 (2.50), 8.087 (6.13), 8.092 (3.65), 8.098 (5.90), 8.105 (2.31), 8.110 (5.40), 8.394 (1.81), 8.410 (3.72), 8.425 (1.72), 8.467 (16.00), 10.646 (0.69), 11.680 (0.64).

### Example 82

### ent-N-[4-butyl-2,5-dioxoimidazolidin-4-yl)methyl]-2-(4-fluorophenyl)-2H-1,2,3-triazole-4-carboxamide

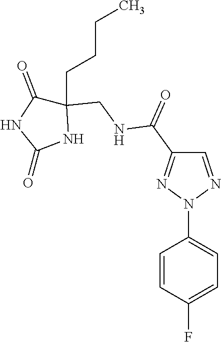 [SMILES: CCCCC1(CNC(=O)c2cnn(-c3ccc(F)cc3)n2)NC(=O)NC1=O]

Enantiomeric separation of rac-N-[4-butyl-2,5-dioxoimidazolidin-4-yl)methyl]-2-(4-fluorophenyl)-2H-1,2,3-triazole-4-carboxamide (50 mg) using method 15D afforded 21 mg of desired product.

LC-MS (Method 9): Rₜ=1.18 min; MS (ESIpos): m/z=375 [M+H]⁺

HPLC (Methode 15E): Rₜ=6.84 min, >99.0% ee

¹H-NMR (400 MHz, DMSO-d6) δ [ppm]: −0.008 (2.71), 0.008 (2.27), 0.832 (6.56), 0.850 (15.73), 0.868 (8.35), 1.008 (0.59), 1.021 (1.08), 1.039 (1.60), 1.051 (1.51), 1.069 (1.30), 1.083 (0.80), 1.102 (0.62), 1.139 (0.51), 1.157 (0.96), 1.175 (0.57), 1.213 (0.97), 1.233 (3.02), 1.249 (5.25), 1.263 (6.07), 1.275 (5.21), 1.290 (2.08), 1.557 (0.83), 1.567 (0.99), 1.592 (2.24), 1.621 (1.95), 1.631 (1.75), 1.640 (1.55), 1.652 (1.46), 1.668 (1.90), 1.702 (0.89), 1.714 (0.51), 3.496 (1.17), 3.512 (1.43), 3.530 (4.37), 3.547 (5.95), 3.565 (4.25), 3.583 (1.26), 3.600 (1.13), 7.451 (0.69), 7.459 (5.97), 7.465 (2.13), 7.481 (10.41), 7.498 (2.30), 7.503 (6.39), 7.512 (0.67), 7.775 (9.36), 8.074 (0.78), 8.083 (6.25), 8.088 (2.69), 8.095 (6.59), 8.100 (3.95), 8.106 (6.39), 8.112 (2.49), 8.118 (5.89), 8.126 (0.59), 8.472 (16.00), 8.502 (2.08), 8.518 (4.31), 8.534 (1.98), 10.673 (3.30).

### Example 83

### ent-N-[(4-cyclopentyl-2,5-dioxoimidazolidin-4-yl)methyl]-2-phenyl-2H-1,2,3-triazole-4-carboxamide

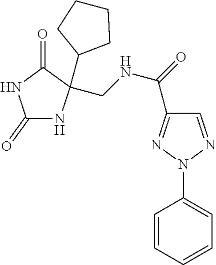 [SMILES: O=C1NC(=O)C(CNC(=O)c2cnn(-c3ccccc3)n2)(C2CCCC2)N1]

Enantiomeric separation of rac-N-[(4-cyclopentyl-2,5-dioxoimidazolidin-4-yl)methyl]-2-phenyl-2H-1,2,3-triazole-4-carboxamide (17 mg) using method 16D afforded 8 mg of desired product.

LC-MS (Method 7): Rₜ=1.46 min; MS (ESIpos): m/z=369 [M+H]⁺

HPLC (Methode 16E): Rₜ=3.39 min, >99.0% ee

¹H-NMR (400 MHz, DMSO-d6) δ [ppm]: 0.853 (0.48), 1.235 (3.04), 1.357 (1.47), 1.488 (4.58), 1.504 (4.51), 1.532 (3.60), 1.769 (1.59), 2.171 (0.60), 2.192 (1.44), 2.213 (1.94), 2.232 (1.34), 2.329 (0.41), 2.671 (0.48), 3.509 (0.42), 3.612 (8.08), 3.628 (8.19), 7.486 (1.87), 7.504 (4.90), 7.523 (3.30), 7.605 (6.00), 7.625 (8.87), 7.644 (4.60), 7.845 (7.68), 8.054 (8.85), 8.074 (8.54), 8.383 (1.85), 8.399 (3.84), 8.414 (1.85), 8.463 (16.00), 10.653 (2.77).

### Example 84

### dia1-N-{[4-(-oxan-3-yl)-2,5-dioxoimidazolidin-4-yl]methyl}-2-phenyl-2H-1,2,3-triazole-4-carboxamide

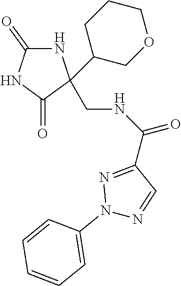 [SMILES: O=C1NC(=O)C(CNC(=O)c2cnn(-c3ccccc3)n2)(C2CCCOC2)N1]

Enantiomeric separation of rac-N-{[4-(-oxan-3-yl)-2,5-dioxoimidazolidin-4-yl]methyl}-2-phenyl-2H-1,2,3-triazole-4-carboxamide (60 mg) using method 17D afforded 14 mg of desired product.

LC-MS (Method 7): Rₜ=1.19 min; MS (ESIpos): m/z=385 [M+H]⁺

HPLC (Methode 17E): Rₜ=6.76 min, >99.0% ee

¹H-NMR (400 MHz, DMSO-d6) δ[ppm]: −0.150 (0.51), 0.146 (0.50), 0.854 (0.45), 1.237 (2.18), 1.282 (3.21), 1.312 (3.43), 1.341 (1.54), 1.479 (2.78), 1.509 (3.01), 1.600 (4.87), 1.631 (3.18), 1.931 (8.00), 1.955 (5.68), 2.328 (0.58), 2.365 (0.47), 2.670 (0.78), 2.709 (0.62), 3.126 (2.81), 3.155 (5.34), 3.184 (3.09), 3.209 (3.65), 3.236 (6.69), 3.263 (3.97), 3.552 (2.60), 3.567 (3.08), 3.587 (5.36), 3.602 (5.12), 3.649 (4.77), 3.665 (5.21), 3.682 (3.04), 3.700 (2.74), 3.770 (8.82), 3.792 (8.38), 7.488 (2.89), 7.506 (7.18), 7.525 (5.26), 7.607 (8.63), 7.627 (14.56), 7.646 (7.60), 7.870 (11.47), 8.059 (13.78), 8.079 (13.01), 8.430 (6.92), 8.445 (4.07), 8.470 (16.00), 10.748 (5.19).

### Example 85

### dia2-N-{[4-(-oxan-3-yl)-2,5-dioxoimidazolidin-4-yl]methyl}-2-phenyl-2H-1,2,3-triazole-4-carboxamide

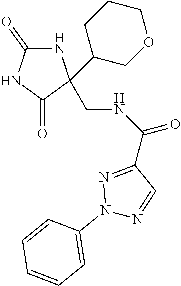 [SMILES: O=C1NC(=O)C(CNC(=O)c2cnn(-c3ccccc3)n2)(C2CCCOC2)N1]

Enantiomeric separation of rac-N-{[4-(-oxan-3-yl)-2,5-dioxoimidazolidin-4-yl]methyl}-2-phenyl-2H-1,2,3-triazole-4-carboxamide (60 mg) using method 17D afforded 8 mg of desired product.

LC-MS (Method 7): Rₜ=1.19 min; MS (ESIpos): m/z=385 [M+H]⁺

HPLC (Methode 17E): Rₜ=7.50 min, >99.0% ee

¹H-NMR (400 MHz, DMSO-d6) δ [ppm]: −0.151 (1.27), 0.144 (1.51), 0.849 (2.75), 1.018 (2.01), 1.177 (3.69), 1.236 (8.97), 1.449 (4.87), 1.560 (6.20), 1.603 (5.93), 1.950 (4.22), 2.245 (2.21), 2.328 (1.77), 2.669 (2.45), 3.153 (8.71), 3.180 (9.86), 3.207 (4.34), 3.609 (12.84), 3.622 (13.46), 3.765 (4.78), 3.793 (4.55), 4.004 (4.99), 4.029 (4.84), 7.508 (7.17), 7.524 (5.61), 7.607 (9.21), 7.626 (14.61), 7.645 (7.82), 7.846 (7.29), 8.057 (14.55), 8.077 (13.67), 8.408 (6.91), 8.469 (16.00), 10.743 (2.21).

### Example 86

### ent-N-{[4-cyclobutyl-2,5-dioxoimidazolidin-4-yl]methyl}-2-(2,4-difluorophenyl)-2H-1,2,3-triazole-4-carboxamide

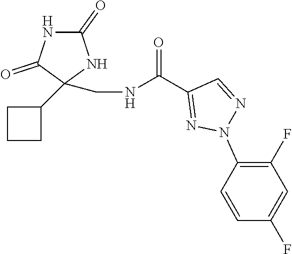 [SMILES: O=C1NC(=O)C(CNC(=O)c2cnn(-c3ccc(F)cc3F)n2)(C2CCC2)N1]

To a solution of ent-5-(aminomethyl)-5-cyclobutylimidazolidine-2,4-dione hydrochloride (32.5 mg, 148 μmol) and 2-(2,4-difluorophenyl)-2H-1,2,3-triazole-4-carboxylic acid (33.4 mg, 148 μmol) in dichloromethane (3 ml) was added 1-ethyl-3-(3-dimethylaminopropyl)carbodiimide hydrochloride (36.9 mg, 193 μmol), 1-hydroxybenzotriazole hydrate (29.5 mg, 193 μmol) and N,N-diisopropylethylamine (72 μl, 410 μmol). The reaction mixture was stirred overnight at RT and was diluted with a small amount of water and acetonitrile. The resulting mixture was purified by preparative HPLC (Method 1f) The combined organic phases were concentrated in vacuo and the remaining TFA was removed, using a Stratospheres PL-HCO3 MP SPE cartridge. After lyophilization, 33.3 mg (58% yield) of the desired product were obtained.

LC-MS (Method 7): Rₜ=1.33 min; MS (ESIpos): m/z=391 [M+H]⁺

¹H-NMR (500 MHz, DMSO-d6) δ [ppm]: −0.007 (2.12), 0.006 (1.50), 1.644 (1.24), 1.660 (1.23), 1.681 (0.85), 1.696 (1.14), 1.710 (1.33), 1.717 (1.30), 1.733 (0.63), 1.743 (0.74), 1.761 (1.71), 1.779 (2.01), 1.793 (2.05), 1.800 (2.08), 1.818 (1.32), 1.835 (0.80), 1.881 (0.99), 1.887 (1.28), 1.895 (1.99), 1.902 (1.83), 1.914 (2.27), 1.933 (1.70), 1.950 (0.62), 2.668 (0.43), 2.686 (1.27), 2.703 (1.98), 2.720 (1.27), 3.481 (7.11), 3.494 (7.01), 7.353 (1.05), 7.356 (1.14), 7.359 (1.07), 7.369 (2.00), 7.372 (2.09), 7.374 (1.99), 7.387 (1.11), 7.390 (1.16), 7.393 (1.03), 7.659 (1.55), 7.664 (1.55), 7.677 (1.77), 7.681 (2.52), 7.686 (1.62), 7.699 (1.55), 7.704 (1.49), 7.931 (5.37), 7.943 (1.95), 7.949 (2.92), 7.961 (2.85), 7.967 (1.58), 7.979 (1.43), 8.383 (1.54), 8.396 (3.16), 8.408 (1.45), 8.508 (16.00), 10.640 (1.34).

### Example 87

### rac-2-(2,4-difluorophenyl)-N-[(4-isopropyl-2,5-dioxoimidazolidin-4-yl)methyl]-2H-1,2,3-triazole-4-carboxamide

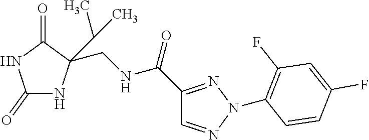 [SMILES: CC(C)C1(CNC(=O)c2cnn(-c3ccc(F)cc3F)n2)NC(=O)NC1=O]

To a solution of rac-5-(aminomethyl)-5-isopropylimidazolidine-2,4-dione hydrochloride (40.0 mg, 193 μmol) and 2-(2,4-difluorophenyl)-2H-1,2,3-triazole-4-carboxylic acid (43.4 mg, 193 μmol) in dichloromethane (4 ml) was added 1-ethyl-3-(3-dimethylaminopropyl)carbodiimide hydrochloride (48.0 mg, 250 μmol), 1-hydroxybenzotriazole hydrate (38.3 mg, 250 μmol) and N,N-diisopropylethylamine (94 μl, 540 μmol). The reaction mixture was stirred overnight at RT and was diluted with a small amount of water and acetonitrile. The resulting mixture was purified by preparative HPLC (Method 2f). The combined organic phases were dried over sodium sulfate, filtered, and concentrated in vacuo, affording 44.9 mg of the desired product (96% purity, 59% yield)

LC-MS (Method 7): Rₜ=1.29 min; MS (ESIpos): m/z=379 [M+H]⁺

¹H-NMR (600 MHz, DMSO-d6) δ [ppm]: 0.835 (15.42), 0.847 (15.44), 0.943 (15.28), 0.955 (15.19), 1.966 (0.94), 1.977 (2.35), 1.989 (3.10), 2.000 (2.19), 2.011 (0.82), 3.601 (0.51), 3.612 (0.79), 3.625 (5.84), 3.627 (5.57), 3.635 (5.22), 3.638 (5.28), 3.650 (0.50), 3.661 (0.44), 7.359 (1.34), 7.361 (1.41), 7.374 (2.47), 7.376 (2.40), 7.387 (1.30), 7.389 (1.29), 7.667 (1.71), 7.672 (1.72), 7.683 (2.04), 7.686 (2.89), 7.690 (1.88), 7.701 (1.70), 7.705 (1.60), 7.799 (6.03), 7.937 (1.60), 7.947 (1.86), 7.952 (3.02), 7.962 (3.02), 7.967 (1.69), 7.977 (1.48), 8.309 (1.97), 8.320 (3.91), 8.330 (1.84), 8.515 (16.00), 8.568 (0.51), 10.652 (5.21).

### Example 88

### ent-2-(2,4-difluorophenyl)-N-[(4-isopropyl-2,5-dioxoimidazolidin-4-yl)methyl]-2H-1,2,3-triazole-4-carboxamide

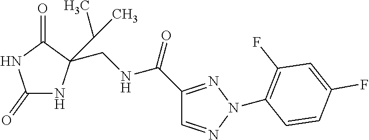 [SMILES: CC(C)C1(CNC(=O)c2cnn(-c3ccc(F)cc3F)n2)NC(=O)NC1=O]

Enantiomeric separation of rac-2-(2,4-difluorophenyl)-N-[(4-isopropyl-2,5-dioxoimidazolidin-4-yl)methyl]-2H-1,2,3-triazole-4-carboxamide by preparative chiral HPLC [sample preparation: 42.3 mg dissolved in a mixture of methanol (3 ml) and acetonitrile (2 ml); injection volume: 0.3 ml; column: Daicel Chiralpak AD-H 5 μm, 250×20 mm; eluent: 50% n-heptane/50% ethanol; flow rate: 15 ml/min; temperature: 40° C.; UV detection: 210 nm] afforded 12.6 mg of the desired product. (98% purity)

Analytical chiral HPLC: Rₜ=4.77 min, e.e. =>99% [column: Chiraltek AD-H 3 μm, 100×4.6 mm; eluent: 50% n-heptane/50% ethanol; flow rate: 1 ml/min; UV detection: 220 nm]

LC-MS (Method 7): Rₜ=1.29 min; MS (ESIpos): m/z=379 [M+H]⁺

¹H-NMR (600 MHz, DMSO-d6) δ [ppm]: 0.835 (16.00), 0.847 (15.94), 0.943 (15.68), 0.954 (15.69), 1.102 (0.43), 1.234 (0.58), 1.966 (1.02), 1.977 (2.50), 1.989 (3.23), 2.000 (2.33), 2.011 (0.85), 2.426 (0.42), 3.601 (0.56), 3.612 (0.88), 3.625 (6.18), 3.627 (5.99), 3.635 (5.64), 3.638 (5.62), 3.650 (0.57), 3.662 (0.48), 7.361 (1.44), 7.374 (2.69), 7.389 (1.44), 7.667 (1.73), 7.672 (1.78), 7.682 (2.10), 7.686 (3.04), 7.690 (2.02), 7.700 (1.72), 7.705 (1.65), 7.803 (8.89), 7.937 (1.64), 7.947 (1.93), 7.952 (3.13), 7.962 (3.12), 7.967 (1.82), 7.976 (1.55), 8.312 (2.06), 8.323 (4.09), 8.333 (2.00), 8.516 (15.77).

### Example 89

### ent-2-(4-chloro-3-fluorophenyl)-N-[(4-cyclobutyl-2,5-dioxoimidazolidin-4-yl)methyl]-2H-1,2,3-triazole-4-carboxamide

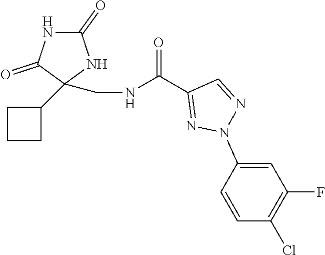 [SMILES: O=C1NC(=O)C(CNC(=O)c2cnn(-c3ccc(Cl)c(F)c3)n2)(C2CCC2)N1]

To a solution of ent-5-(aminomethyl)-5-cyclobutylimidazolidine-2,4-dione hydrochloride (28.1 mg, 128 μmol) and 2-(4-chloro-3-fluorophenyl)-2H-1,2,3-triazole-4-carboxylicacid (30.9 mg, 128 μmol) in dichloromethane (2.6 ml) was added 1-ethyl-3-(3-dimethylaminopropyl)carbodiimide hydrochloride (31.8 mg, 166 μmol), 1-hydroxybenzotriazole hydrate (25.4 mg, 166 μmol) and N,N-diisopropylethylamine (62 μl, 360 μmol). The reaction mixture was stirred overnight at RT and concentrated under reduced pressure. The crude product was purified twice by preparative HPLC (Method 1f) The combined organic phases were concentrated in vacuo and the remaining TFA was removed, using a Stratospheres PL-HCO3 MP SPE cartridge. After lyophilization, 10.6 mg (20% yield) of the desired product was obtained.

LC-MS (Method 7): Rₜ=1.60 min; MS (ESIpos): m/z=407 [M+H]⁺

¹H-NMR (500 MHz, DMSO-d₆) δ[ppm]: −0.120 (0.69), 0.116 (0.62), 1.663 (1.65), 1.685 (1.09), 1.720 (1.60), 1.753 (0.95), 1.772 (1.91), 1.789 (2.93), 1.803 (2.92), 1.817 (1.83), 1.836 (0.93), 1.908 (2.60), 1.926 (2.62), 1.945 (1.91), 1.963 (0.88), 2.096 (1.05), 2.361 (0.59), 2.634 (0.56), 2.691 (1.52), 2.709 (2.26), 2.726 (1.44), 3.432 (1.47), 3.443 (1.62), 3.459 (3.16), 3.471 (2.83), 3.501 (2.63), 3.514 (2.80), 3.528 (1.45), 3.542 (1.34), 7.857 (2.42), 7.875 (4.76), 7.891 (4.11), 7.908 (1.74), 7.929 (4.14), 7.934 (4.05), 7.947 (2.36), 8.069 (3.26), 8.073 (3.11), 8.089 (3.21), 8.093 (3.02), 8.509 (1.98), 8.521 (16.00), 8.533 (1.78), 8.561 (0.50), 10.646 (1.11).

### Example 90

### ent-N-[(4-cyclobutyl-2,5-dioxoimidazolidin-4-yl)methyl]-2-(3,4,5-trifluorophenyl)-2H-1,2,3-triazole-4-carboxamide

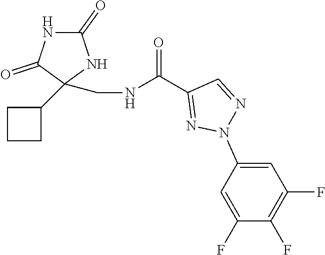 [SMILES: O=C1NC(=O)C(CNC(=O)c2cnn(-c3cc(F)c(F)c(F)c3)n2)(C2CCC2)N1]

To a solution of ent-5-(aminomethyl)-5-cyclobutylimidazolidine-2,4-dione hydrochloride (80.0 mg, 364 μmol) and 2-(3,4,5-trifluorophenyl)-2H-1,2,3-triazole-4-carboxylic acid (88.5 mg, 364 μmol) in dichloromethane (7.5 ml) was added 1-ethyl-3-(3-dimethylaminopropyl)carbodiimide hydrochloride (90.8 mg, 473 μmol), 1-hydroxybenzotriazole hydrate (72.5 mg, 473 μmol) and N,N-diisopropylethylamine (180 μl, 1.0 mmol). The reaction mixture was stirred overnight at RT and concentrated under reduced pressure. The crude product was purified by preparative HPLC (Method 2f) The combined organic phases were concentrated in vacuo and the remaining TFA was removed, using a Stratospheres PL-HCO3 MP SPE cartridge. After lyophilization, 57.5 mg (39% yield) of the desired product were obtained.

LC-MS (Method 8): Rₜ=0.86 min; MS (ESIpos): m/z=409 [M+H]⁺

¹H-NMR (500 MHz, DMSO-d6) δ [ppm]: −0.007 (1.59), 0.006 (1.18), 1.649 (1.18), 1.661 (1.29), 1.698 (1.12), 1.709 (1.21), 1.716 (1.30), 1.732 (0.60), 1.747 (0.79), 1.765 (1.53), 1.784 (2.03), 1.795 (1.97), 1.802 (2.22), 1.818 (1.29), 1.836 (0.75), 1.888 (1.19), 1.897 (1.15), 1.904 (1.52), 1.920 (0.96), 1.929 (1.67), 1.949 (1.52), 1.969 (0.67), 2.687 (1.25), 2.704 (1.84), 2.721 (1.19), 3.409 (1.56), 3.420 (1.67), 3.436 (2.52), 3.448 (2.19), 3.512 (2.17), 3.526 (2.29), 3.540 (1.49), 3.553 (1.38), 7.939 (1.96), 8.008 (3.19), 8.021 (3.73), 8.025 (3.73), 8.037 (3.27), 8.549 (16.00), 8.564 (2.75), 8.577 (1.35), 10.669 (0.80).

### Example 91

### ent-2-(3-chloro-4-fluorophenyl)-N-[(4-cyclobutyl-2,5-dioxoimidazolidin-4-yl)methyl]-2H-1,2,3-triazole-4-carboxamide

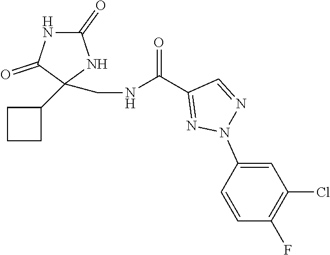 [SMILES: O=C1NC(=O)C(CNC(=O)c2cnn(-c3ccc(F)c(Cl)c3)n2)(C2CCC2)N1]

To a solution of ent-5-(aminomethyl)-5-cyclobutylimidazolidine-2,4-dione hydrochloride (19.0 mg, 86.3 μmol) and 2-(3-chloro-4-fluorophenyl)-2H-1,2,3-triazole-4-carboxylic acid (20.9 mg, 86.3 μmol) in dichloromethane (1.8 ml) was added 1-ethyl-3-(3-dimethylaminopropyl)carbodiimide hydrochloride (21.5 mg, 112 μmol), 1-hydroxybenzotriazole hydrate (17.2 mg, 112 μmol) and N,N-diisopropylethylamine (42 μl, 240 μmol). The reaction mixture was stirred overnight at RT and was diluted with a small amount of Water and acetonitrile. The resulting mixture was purified by preparative HPLC (Method 1f). A second purification by preparative chiral HPLC [sample preparation: 25 mg dissolved in a mixture of ethanol (1 ml) and DCM (1 ml); injection volume: 0.4 ml; column: Daicel Chiralpak IG 5 μm, 250×20 mm; eluent: n-heptan/ethanol 50:50; flow rate: 15 ml/min; temperature: 50° C.; UV detection: 220 nm] gave 21.0 mg (60% yield) of the desired compound.

LC-MS (Method 7): Rₜ=1.58 min; MS (ESIpos): m/z=407 [M+H]⁺

¹H-NMR (500 MHz, DMSO-d6) δ [ppm]: 1.651 (1.13), 1.667 (1.15), 1.691 (0.77), 1.708 (1.05), 1.722 (1.20), 1.728 (1.22), 1.744 (0.60), 1.752 (0.77), 1.769 (1.60), 1.787 (1.84), 1.801 (1.90), 1.808 (1.88), 1.827 (1.24), 1.844 (0.71), 1.896 (0.92), 1.902 (1.16), 1.917 (1.75), 1.932 (2.25), 1.950 (1.52), 1.969 (0.62), 2.077 (1.16), 2.679 (0.41), 2.697 (1.15), 2.714 (1.82), 2.731 (1.16), 3.305 (0.53), 3.352 (0.83), 3.430 (1.45), 3.442 (1.63), 3.457 (2.61), 3.469 (2.31), 3.520 (2.35), 3.533 (2.52), 3.547 (1.54), 3.561 (1.43), 7.692 (2.87), 7.710 (5.84), 7.728 (3.10), 7.999 (6.61), 8.053 (1.69), 8.058 (1.99), 8.061 (1.95), 8.066 (1.86), 8.071 (1.69), 8.076 (1.95), 8.079 (1.71), 8.084 (1.60), 8.248 (3.00), 8.253 (2.93), 8.261 (3.04), 8.266 (2.78), 8.505 (16.00), 8.519 (2.95), 8.532 (1.45), 10.671 (3.77).

### Example 92

### ent-2-(3-cyanophenyl)-N-[(4-cyclobutyl-2,5-dioxoimidazolidin-4-yl)methyl]-2H-1,2,3-triazole-4-carboxamide

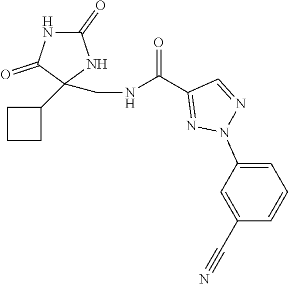 [SMILES: N#Cc1cccc(-n2ncc(C(=O)NCC3(C4CCC4)NC(=O)NC3=O)n2)c1]

To a solution of ent-5-(aminomethyl)-5-cyclobutylimidazolidine-2,4-dione hydrochloride (51.3 mg, 233 μmol) and 2-(3-cyanophenyl)-2H-1,2,3-triazole-4-carboxylic acid (50.0 mg, 233 μmol) in DMF (1.5 ml) was added 1-ethyl-3-(3-dimethylaminopropyl)carbodiimide hydrochloride (58.2 mg, 303 μmol), 1-hydroxybenzotriazole hydrate (46.5 mg, 303 μmol) and N,N-diisopropylethylamine (110 μl, 650 μmol). The reaction mixture was stirred for 3 days at RT and concentrated under reduced pressure. The crude product was purified by preparative HPLC (Method 1f) The combined organic phases were concentrated in vacuo and the remaining TFA was removed, using a Stratospheres PL-HCO3 MP SPE cartridge. After lyophilization, 38.1 mg (99% purity, 43% yield) of the desired product were obtained.

LC-MS (Method 7): Rₜ=1.30 min; MS (ESIpos): m/z=380 [M+H]⁺

¹H-NMR (600 MHz, DMSO-d6) δ [ppm]: 1.646 (1.35), 1.659 (1.36), 1.680 (0.77), 1.700 (1.21), 1.715 (1.41), 1.755 (0.60), 1.771 (1.66), 1.786 (2.48), 1.794 (2.23), 1.800 (2.56), 1.807 (1.36), 1.814 (1.43), 1.828 (0.80), 1.887 (1.15), 1.891 (1.34), 1.898 (1.28), 1.905 (1.64), 1.913 (1.24), 1.929 (1.84), 1.945 (1.69), 1.962 (0.81), 2.071 (0.69), 2.515 (0.77), 2.518 (0.77), 2.521 (0.79), 2.679 (0.49), 2.694 (1.38), 2.708 (2.10), 2.722 (1.35), 3.435 (1.70), 3.445 (1.82), 3.458 (2.93), 3.468 (2.59), 3.505 (2.54), 3.515 (2.67), 3.527 (1.56), 3.538 (1.41), 7.827 (2.96), 7.840 (5.79), 7.854 (3.63), 7.899 (1.75), 7.972 (3.06), 7.974 (4.03), 7.976 (2.88), 7.985 (2.57), 7.987 (3.33), 7.989 (2.27), 8.367 (2.66), 8.369 (2.94), 8.371 (2.93), 8.372 (2.72), 8.381 (2.53), 8.382 (2.59), 8.385 (2.79), 8.386 (2.38), 8.465 (4.02), 8.467 (5.54), 8.470 (3.31), 8.540 (16.00), 8.551 (1.48), 8.562 (2.66), 8.572 (1.34).

### Example 93

### ent-N-[(4-cyclobutyl-2,5-dioxoimidazolidin-4-yl)methyl]-2-(3-fluorophenyl)-2H-1,2,3-triazole-4-carboxamide

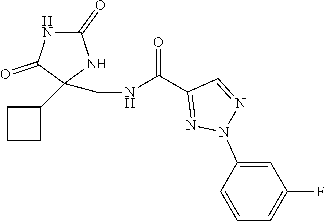 [SMILES: O=C1NC(=O)C(CNC(=O)c2cnn(-c3cccc(F)c3)n2)(C2CCC2)N1]

To a solution of ent-5-(aminomethyl)-5-cyclobutylimidazolidine-2,4-dione hydrochloride (53.0 mg, 241 μmol) and 2-(3-fluorophenyl)-2H-1,2,3-triazole-4-carboxylic acid (50.0 mg, 241 μmol) in dichloromethane (5 ml) was added 1-ethyl-3-(3-dimethylaminopropyl)carbodiimide hydrochloride (60.1 mg, 314 μmol), 1-hydroxybenzotriazole hydrate (48.0 mg, 314 μmol) and N,N-diisopropylethylamine (120 μl, 680 μmol). The reaction mixture was stirred overnight at RT, after which extra portion of N,N-diisopropylethylamine (120 μl, 680 μmol) and DMF (1.5 ml) were added due to incomplete conversion. After stirring for 24 h at RT and for 4 h at 40° C., the reaction mixture was additionally stirred for 24 h at RT. The resulting reaction mixture was concentrated under reduced pressure and the crude product was purified by preparative HPLC (Method 3f). After lyophilization, 15.1 mg (17% yield) of the desired product were obtained.

LC-MS (Method 7): Rₜ=1.41 min; MS (ESIpos): m/z=373 [M+H]⁺

¹H-NMR (600 MHz, DMSO-d6) δ [ppm]: 1.656 (1.35), 1.669 (1.21), 1.685 (0.57), 1.714 (1.14), 1.726 (1.38), 1.732 (1.35), 1.745 (0.60), 1.762 (0.67), 1.777 (1.84), 1.792 (2.31), 1.805 (2.16), 1.813 (2.13), 1.827 (1.42), 1.842 (0.82), 1.903 (1.06), 1.908 (1.28), 1.920 (2.27), 1.935 (2.59), 1.951 (1.77), 1.966 (0.71), 2.521 (0.60), 2.691 (0.46), 2.706 (1.35), 2.720 (2.06), 2.734 (1.38), 3.450 (1.77), 3.460 (1.95), 3.472 (3.19), 3.483 (2.80), 3.522 (2.87), 3.533 (3.02), 3.545 (1.81), 3.556 (1.63), 6.573 (0.43), 7.358 (1.17), 7.362 (1.31), 7.372 (2.34), 7.376 (2.45), 7.386 (1.24), 7.390 (1.28), 7.664 (1.53), 7.675 (1.77), 7.678 (3.05), 7.688 (2.98), 7.692 (1.77), 7.702 (1.53), 7.871 (1.63), 7.875 (2.77), 7.879 (1.84), 7.887 (1.67), 7.891 (2.73), 7.895 (1.77), 7.922 (3.12), 7.924 (2.66), 7.935 (2.66), 7.938 (2.41), 7.995 (5.14), 8.469 (1.77), 8.480 (3.33), 8.491 (1.88), 8.499 (16.00), 10.668 (4.22).

### Example 94

### ent-N-[(4-cyclobutyl-2,5-dioxoimidazolidin-4-yl)methyl]-2-(3,4-difluorophenyl)-2H-1,2,3-triazole-4-carboxamide

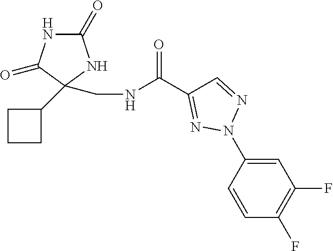 [SMILES: O=C1NC(=O)C(CNC(=O)c2cnn(-c3ccc(F)c(F)c3)n2)(C2CCC2)N1]

To a solution of ent-5-(aminomethyl)-5-cyclobutylimidazolidine-2,4-dione hydrochloride (53.0 mg, 241 μmol) and 2-(3,4-difluorophenyl)-2H-1,2,3-triazole-4-carboxylic acid (54.3 mg, 241 μmol) in DCM (5 ml) was added 1-ethyl-3-(3-dimethylaminopropyl)carbodiimide hydrochloride (60.1 mg, 314 μmol), 1-hydroxybenzotriazole hydrate (48.0 mg, 314 μmol) and N,N-diisopropylethylamine (120 μl, 680 μmol). The reaction mixture was stirred overnight at RT, after which extra portion of N,N-diisopropylethylamine (120 μl, 680 μmol) and DMF (1.5 ml) were added due to incomplete conversion. After stirring for 24 h at RT, the reaction mixture was additionally stirred for 8 h at 40° C. The resulting reaction mixture was concentrated under reduced pressure and the crude product was purified by preparative HPLC (Method 3f). After lyophilization, 34.4 mg (37% yield) of the desired product were obtained.

LC-MS (Method 7): Rₜ=1.47 min; MS (ESIpos): m/z=391 [M+H]⁺

¹H-NMR (600 MHz, DMSO-d6) δ [ppm]: 1.653 (1.23), 1.667 (1.12), 1.712 (1.06), 1.723 (1.29), 1.729 (1.23), 1.742 (0.50), 1.760 (0.67), 1.775 (1.68), 1.790 (2.07), 1.803 (1.96), 1.811 (1.90), 1.825 (1.29), 1.840 (0.73), 1.899 (1.01), 1.905 (1.17), 1.918 (2.18), 1.933 (2.24), 1.949 (1.57), 1.966 (0.62), 2.425 (0.50), 2.524 (1.06), 2.655 (0.50), 2.686 (0.45), 2.701 (1.23), 2.715 (1.90), 2.729 (1.29), 3.439 (1.68), 3.450 (1.85), 3.462 (2.85), 3.473 (2.52), 3.521 (2.52), 3.532 (2.69), 3.543 (1.73), 3.555 (1.57), 7.712 (1.06), 7.727 (2.18), 7.744 (2.24), 7.758 (1.29), 7.915 (1.79), 7.922 (1.12), 7.931 (1.57), 7.988 (4.64), 8.102 (1.23), 8.106 (1.23), 8.114 (1.40), 8.118 (1.51), 8.120 (1.45), 8.125 (1.29), 8.132 (1.29), 8.136 (1.12), 8.465 (1.62), 8.476 (3.02), 8.487 (1.68), 8.498 (16.00), 10.663 (3.97).

### Example 95

### ent-2-(4-chlorophenyl)-N-[(4-cyclobutyl-2,5-dioxoimidazolidin-4-yl)methyl]-2H-1,2,3-triazole-4-carboxamide

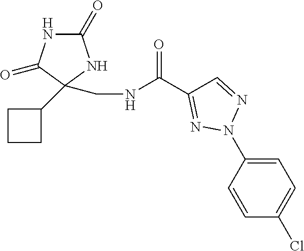 [SMILES: O=C1NC(=O)C(CNC(=O)c2cnn(-c3ccc(Cl)cc3)n2)(C2CCC2)N1]

To a solution of ent-5-(aminomethyl)-5-cyclobutylimidazolidine-2,4-dione hydrochloride (49.1 mg, 224 μmol) and 2-(4-chlorophenyl)-2H-1,2,3-triazole-4-carboxylic acid (50.0 mg, 224 μmol) in DMF (1.4 ml) was added 1-ethyl-3-(3-dimethylaminopropyl)carbodiimide hydrochloride (55.7 mg, 291 μmol), 1-hydroxybenzotriazole hydrate (44.5 mg, 291 μmol) and N,N-diisopropylethylamine (110 μl, 630 μmol). After stirring for 3 days at RT, the reaction mixture was stirred for 6 h at 40° C. The resulting reaction mixture was concentrated under reduced pressure and the crude product was purified by preparative HPLC (Method 4f). After lyophilization, 35.1 mg (40% yield) of the desired product were obtained.

LC-MS (Method 7): Rₜ=1.54 min; MS (ESIpos): m/z=389 [M+H]⁺

¹H-NMR (600 MHz, DMSO-d6) δ [ppm]: 1.653 (1.44), 1.667 (1.26), 1.682 (0.61), 1.711 (1.19), 1.723 (1.44), 1.728 (1.41), 1.742 (0.61), 1.758 (0.69), 1.774 (1.91), 1.789 (2.38), 1.802 (2.24), 1.810 (2.31), 1.825 (1.48), 1.839 (0.83), 1.900 (1.12), 1.905 (1.34), 1.917 (2.17), 1.931 (2.71), 1.947 (1.84), 1.962 (0.69), 2.689 (0.47), 2.703 (1.41), 2.717 (2.17), 2.731 (1.44), 3.455 (1.63), 3.465 (1.84), 3.477 (3.54), 3.488 (3.03), 3.511 (3.03), 3.522 (3.29), 3.534 (1.66), 3.545 (1.48), 7.693 (1.41), 7.698 (9.97), 7.701 (3.11), 7.709 (3.68), 7.713 (10.47), 7.718 (1.05), 7.990 (5.38), 8.067 (1.63), 8.072 (11.45), 8.075 (3.36), 8.083 (3.61), 8.087 (10.08), 8.092 (1.01), 8.445 (1.84), 8.456 (3.54), 8.466 (1.77), 8.485 (16.00), 10.665 (4.55).

### Example 96

### ent-2-(3-chlorophenyl)-N-[(4-cyclobutyl-2,5-dioxoimidazolidin-4-yl)methyl]-2H-1,2,3-triazole-4-carboxamide

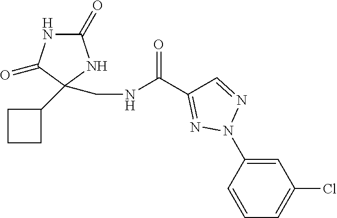 [SMILES: O=C1NC(=O)C(CNC(=O)c2cnn(-c3cccc(Cl)c3)n2)(C2CCC2)N1]

To a solution of ent-5-(aminomethyl)-5-cyclobutylimidazolidine-2,4-dione hydrochloride (49.1 mg, 224 μmol) and 2-(3-chlorophenyl)-2H-1,2,3-triazole-4-carboxylic acid (50.0 mg, 224 μmol) in DMF (1.4 ml) was added 1-ethyl-3-(3-dimethylaminopropyl)carbodiimide hydrochloride (55.7 mg, 291 μmol), 1-hydroxybenzotriazole hydrate (44.5 mg, 291 μmol) and N,N-diisopropylethylamine (110 μl, 630 μmol). After stirring for 3 days at RT, the reaction mixture was stirred for 6 h at 40° C. The resulting reaction mixture was concentrated under reduced pressure and the crude product was purified by preparative HPLC (Method 4f). After lyophilization, 35.1 mg (40% yield) of the desired product were obtained.

LC-MS (Method 7): Rₜ=1.54 min; MS (ESIpos): m/z=389 [M+H]⁺

¹H-NMR (600 MHz, DMSO-d6) δ [ppm]: 1.655 (1.21), 1.669 (1.13), 1.714 (1.05), 1.725 (1.25), 1.731 (1.25), 1.744 (0.56), 1.761 (0.60), 1.776 (1.65), 1.791 (2.10), 1.804 (1.97), 1.813 (1.93), 1.826 (1.29), 1.841 (0.73), 1.902 (0.97), 1.907 (1.17), 1.920 (2.02), 1.934 (2.42), 1.950 (1.61), 1.964 (0.60), 2.689 (0.44), 2.704 (1.25), 2.719 (1.93), 2.733 (1.33), 3.446 (1.57), 3.456 (1.77), 3.469 (2.90), 3.479 (2.50), 3.521 (2.54), 3.532 (2.74), 3.544 (1.65), 3.555 (1.49), 7.577 (1.93), 7.579 (2.26), 7.580 (2.18), 7.582 (2.14), 7.591 (2.74), 7.592 (2.98), 7.594 (3.06), 7.595 (2.78), 7.650 (3.95), 7.663 (6.49), 7.677 (2.94), 7.989 (4.63), 8.033 (2.34), 8.034 (2.70), 8.036 (2.70), 8.038 (2.66), 8.046 (2.18), 8.048 (2.38), 8.049 (2.62), 8.051 (2.42), 8.115 (3.67), 8.118 (6.49), 8.121 (3.22), 8.501 (16.00), 8.509 (3.14), 8.519 (1.49), 10.666 (3.99).

### Example 97

### ent-N-{[2,5-dioxo-4-(pyridin-2-yl)imidazolidin-4-yl]methyl}-2-(4-fluorophenyl)-2H-1,2,3-triazole-4-carboxamide

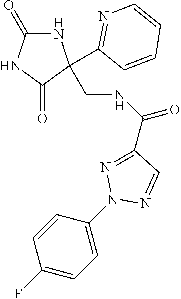 [SMILES: O=C1NC(=O)C(CNC(=O)c2cnn(-c3ccc(F)cc3)n2)(c2ccccn2)N1]

Enantiomeric separation of rac-N-{[2,5-dioxo-4-(pyridin-2-yl)imidazolidin-4-yl]methyl}-2-(4-fluorophenyl)-2H-1,2,3-triazole-4-carboxamide (31.7 mg) by preparative chiral HPLC [column: Daicel Chiralcel® OX-H 5 μm, 250×30 mm; eluent: 50% n-heptane/50% ethanol+0.2% diethylamine; flow rate: 40 ml/min; temperature: 40° C.; UV detection: 220 nm] afforded 13 mg of the desired product. The product was further purified by preparative HPLC (Method 1f) The combined organic phases were concentrated in vacuo and the remaining TFA was removed, using a Stratospheres PL-HCO3 MP SPE cartridge. After lyophilization, 5.9 mg of the desired product were obtained.

Analytical chiral HPLC: Rₜ=7.63 min, e.e. =99% [column: Daicel Chiralcel® OX-H 5 μm, 250×30 mm; eluent: 50% n-heptane/50% ethanol+0.2% diethylamine; flow rate: 40 ml/min; temperature: 40° C.; UV detection: 220 nm]

¹H-NMR (500 MHz, DMSO-d6) δ [ppm]: 0.007 (1.73), 2.094 (0.60), 2.363 (0.49), 2.637 (0.48), 4.079 (1.82), 4.092 (2.00), 4.106 (2.78), 4.119 (2.52), 4.210 (2.40), 4.222 (2.60), 4.237 (1.88), 4.250 (1.66), 7.396 (2.33), 7.405 (2.58), 7.409 (2.61), 7.420 (2.48), 7.461 (5.00), 7.465 (1.90), 7.479 (8.45), 7.491 (1.84), 7.496 (5.24), 7.544 (4.51), 7.560 (4.85), 7.856 (2.15), 7.860 (2.21), 7.872 (3.49), 7.875 (3.52), 7.887 (1.87), 7.891 (1.84), 8.072 (5.19), 8.081 (5.55), 8.086 (3.28), 8.090 (5.39), 8.099 (4.99), 8.338 (1.52), 8.474 (16.00), 8.497 (1.67), 8.510 (3.24), 8.522 (1.58), 8.631 (3.25), 8.639 (3.16), 10.914 (0.96).

### Example 98

### N-{[(4R)-4-cyclopropyl-2,5-dioxoimidazolidin-4-yl]methyl}-2-(3,4,5-trifluorophenyl)-2H-1,2,3-triazole-4-carboxamide

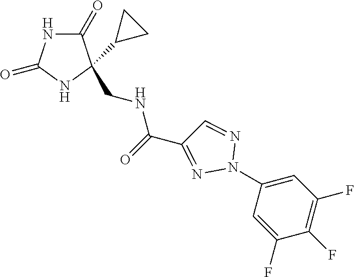 [SMILES: O=C1NC(=O)[C@](CNC(=O)c2cnn(-c3cc(F)c(F)c(F)c3)n2)(C2CC2)N1]

To a solution of (5R)-5-(aminomethyl)-5-cyclopropylimidazolidine-2,4-dione hydrochloride (120 mg, 584 μmol) and 2-(3,4,5-trifluorophenyl)-2H-1,2,3-triazole-4-carboxylic acid (142 mg, 584 μmol) in dichloromethane (12 ml) was added 1-ethyl-3-(3-dimethylaminopropyl)carbodiimide hydrochloride (145 mg, 759 μmol), 1-hydroxybenzotriazole hydrate (116 mg, 759 μmol) and N,N-diisopropylethylamine (285 μl, 1.6 mmol). The reaction mixture was stirred overnight at RT and concentrated under reduced pressure. The crude product was purified by preparative HPLC (Method 2f) The combined organic phases were concentrated in vacuo and the remaining TFA was removed, using a Stratospheres PL-HCO3 MP SPE cartridge. After concentration in vacuo, 116 mg (50% yield) of the desired product were obtained.

LC-MS (Method 7): Rₜ=1.46 min; MS (ESIpos): m/z=395 [M+H]⁺

¹H-NMR (500 MHz, DMSO-d₆) δ[ppm]: 0.117 (0.55), 0.128 (1.16), 0.136 (1.88), 0.146 (1.86), 0.156 (1.47), 0.167 (0.69), 0.310 (0.49), 0.328 (1.26), 0.331 (1.13), 0.340 (1.62), 0.349 (1.68), 0.356 (1.04), 0.365 (0.82), 0.405 (0.55), 0.415 (1.36), 0.426 (2.29), 0.432 (2.67), 0.445 (2.87), 0.456 (2.06), 0.468 (1.12), 1.137 (0.73), 1.148 (1.48), 1.153 (1.53), 1.158 (1.13), 1.164 (2.46), 1.175 (1.44), 1.180 (1.26), 1.191 (0.58), 2.121 (0.57), 3.612 (1.72), 3.624 (1.89), 3.639 (2.76), 3.651 (2.45), 3.731 (2.49), 3.745 (2.64), 3.758 (1.80), 3.772 (1.67), 7.572 (3.77), 8.003 (3.59), 8.016 (4.21), 8.020 (4.10), 8.033 (3.59), 8.543 (16.00), 8.565 (1.56), 8.578 (2.80), 8.590 (1.40).

### Example 99

### rac-N-[(4-isopropyl-2,5-dioxoimidazolidin-4-yl)methyl]-2-(3,4,5-trifluorophenyl)-2H-1,2,3-triazole-4-carboxamide

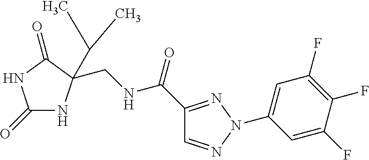 [SMILES: CC(C)C1(CNC(=O)c2cnn(-c3cc(F)c(F)c(F)c3)n2)NC(=O)NC1=O]

To a solution of rac-5-(aminomethyl)-5-(propan-2-yl)imidazolidine-2,4-dione (120 mg, 701 μmol) and 2-(3,4,5-trifluorophenyl)-2H-1,2,3-triazole-4-carboxylic acid (170 mg, 701 μmol) in DCM (14 ml) was added 1-ethyl-3-(3-dimethylaminopropyl)carbodiimide hydrochloride (175 mg, 911 μmol), 1-hydroxybenzotriazole hydrate (140 mg, 911 μmol) and N,N-diisopropylethylamine (340 μl, 2.0 mmol). The reaction mixture was stirred overnight at RT and concentrated under reduced pressure. The crude product was purified by preparative HPLC (Method 2f) The combined organic phases were concentrated in vacuo and the remaining TFA was removed, using a Stratospheres PL-HCO3 MP SPE cartridge. After lyophilization, 53.8 mg (94% purity, 18% yield) of the desired product were obtained.

LC-MS (Method 8): Rₜ=0.83 min; MS (ESIpos): m/z=397 [M+H]⁺

¹H-NMR (500 MHz, DMSO-d6) δ [ppm]: 0.006 (0.93), 0.844 (12.17), 0.857 (12.22), 0.957 (11.98), 0.971 (12.18), 1.974 (0.74), 1.987 (1.88), 2.001 (2.45), 2.014 (1.74), 2.028 (0.65), 2.121 (0.41), 3.546 (1.67), 3.559 (1.82), 3.574 (2.49), 3.587 (2.20), 3.686 (2.29), 3.699 (2.43), 3.714 (1.75), 3.727 (1.60), 7.286 (0.43), 7.861 (0.45), 7.874 (0.53), 7.899 (6.54), 8.014 (3.30), 8.027 (3.92), 8.031 (3.85), 8.044 (3.36), 8.542 (1.73), 8.557 (16.00), 8.567 (1.62), 10.661 (2.15).

### Example 100

### rac-2-(4-fluorophenyl)-N-{[4-(5-methyl-1,3-thiazol-4-yl)-2,5-dioxoimidazolidin-4-yl]methyl}-2H-1,2,3-triazole-4-carboxamide

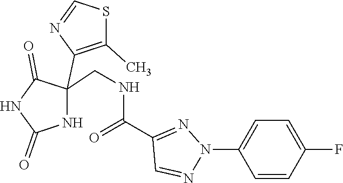 [SMILES: Cc1scnc1C1(CNC(=O)c2cnn(-c3ccc(F)cc3)n2)NC(=O)NC1=O]

2-(4-fluorophenyl)-2H-1,2,3-triazole-4-carboxylic acid (101 mg, 485 μmol) dissolved in 10 ml of dichloromethane was treated with N-ethyl-N-isopropylpropan-2-amine (240 μl, 1.4 mmol), 3-{[(ethylimino)methylene]amino}-N,N-dimethylpropan-1-amine hydrochloride (121 mg, 631 μmol), 1H-benzotriazol-1-ol hydrate (96.6 mg, 631 μmol) and rac-5-(aminomethyl)-5-(5-methyl-1,3-thiazol-4-yl)imidazolidine-2,4-dione hydrochloride (150 mg, 85% purity, 485 μmol). The mixture was stirred over night at room temperature. The solvent was removed on a rotary evaporator. The residue was taken up with ethyl acetate and washed with water and saturated aqueous sodium chloride solution. The organic layer was dried over sodium sulfate, filtered, Isolute® was added and the solvent was evaporated on a rotary evaporator. The crude product was purified by column chromatography (Machine: Biotage® Isolera One; column: Biotage® SNAP Ultra 25 g; gradient: DCM/MeOH-gradient, 2% MeOH-20% MeOH; flow: 75 ml/min). Product containing samples were united and the solvents were removed on a rotary evaporator. 80.0 mg (100% purity, 40% yield) of the title compound were obtained.

LC-MS (Method 8): Rₜ=0.74 min; MS (ESIpos): m/z=416 [M+H]⁺

¹H-NMR (400 MHz, DMSO-d₆) δ[ppm]: −0.008 (2.26), 0.008 (2.45), 2.356 (16.00), 4.187 (0.46), 4.206 (1.38), 4.222 (1.55), 4.226 (1.56), 4.242 (1.42), 4.260 (0.43), 4.277 (0.40), 7.456 (2.11), 7.461 (0.75), 7.478 (3.34), 7.494 (0.75), 7.500 (2.28), 8.073 (2.16), 8.078 (0.88), 8.085 (2.28), 8.090 (1.35), 8.096 (2.30), 8.102 (0.83), 8.107 (2.11), 8.356 (2.84), 8.473 (0.68), 8.483 (7.58), 8.505 (0.67), 8.930 (7.41), 11.027 (2.07).

### Example 101

### N-{[(4R)-4-cyclopropyl-2,5-dioxoimidazolidin-4-yl]methyl}-2-(3-fluoropyridin-2-yl)-2H-1,2,3-triazole-4-carboxamide

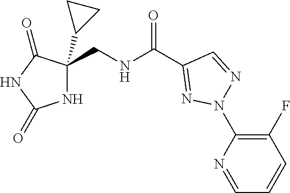 [SMILES: O=C1NC(=O)[C@](CNC(=O)c2cnn(-c3ncccc3F)n2)(C2CC2)N1]

2-(3-fluoropyridin-2-yl)-2H-1,2,3-triazole-4-carboxylic acid (97.0 mg, 83% purity, 387 μmol) dissolved in 5 ml of dichloromethane was treated with N-ethyl-N-isopropylpropan-2-amine (190 μl, 1.1 mmol), 3-{[(ethylimino)methylene]amino}-N,N-dimethylpropan-1-amine hydrochloride (96.4 mg, 503 μmol), 1H-benzotriazol-1-ol hydrate (77.0 mg, 503 μmol) and (5R)-5-(aminomethyl)-5-cyclopropylimidazolidine-2,4-dione hydrochloride (79.5 mg, 387 μmol). The mixture was stirred over night at room temperature. The solvent was removed on a rotary evaporator. The residue was taken up with dimethylformamide and purified by preparative HPLC (Instrument: Waters Prep LC/MS System, column: Phenomenex Kinetex C18 5 μm 100×30 mm; eluent A: water, eluent B: acetonitrile, eluent C: 2% formic acid in water, eluent D: acetonitrile/water (80 Vol. %/20 Vol %) flow rate: 80 ml/min; room temperature, UV-detection 200-400 nm, At-Column Injektion; gradient: eluent A 0 bis 2 min 70 ml, eluent B 0 bis 2 min 0 ml, eluent A 2 bis 10 min from 70 ml to 55 ml and eluent B from 0 ml to 15 ml, 10 bis 12 min 0 ml eluent A and 70 ml eluent B. Eluent C and eluent D constant flow 5 ml/min over the whole runtime). After lyophilization, 3.00 mg (100% purity, 2% yield) of the title compound were obtained.

LC-MS (Method 7): Rₜ=0.95 min; MS (ESIpos): m/z=360 [M+H]⁺

¹H-NMR (400 MHz, DMSO-d6) δ [ppm]: −0.149 (1.88), −0.008 (16.00), 0.008 (15.37), 0.146 (2.20), 0.335 (0.78), 0.402 (1.15), 0.420 (2.09), 0.438 (1.57), 1.146 (0.78), 1.161 (0.84), 2.135 (0.99), 2.323 (1.57), 2.327 (2.25), 2.332 (1.62), 2.366 (1.88), 2.381 (1.10), 2.523 (8.58), 2.665 (1.67), 2.670 (2.41), 2.674 (1.73), 2.710 (1.88), 3.695 (3.03), 3.711 (3.08), 7.602 (2.72), 7.772 (0.73), 7.783 (0.94), 7.793 (1.73), 7.804 (1.10), 7.813 (0.94), 8.163 (0.94), 8.166 (0.99), 8.189 (1.41), 8.210 (0.99), 8.214 (0.94), 8.519 (1.93), 8.530 (2.51), 8.565 (6.80), 10.647 (1.15).

### Example 102

### diamix-N-[(4-sec-butyl-2,5-dioxoimidazolidin-4-yl)methyl]-2-(4-fluorophenyl)-2H-1,2,3-triazole-4-carboxamide

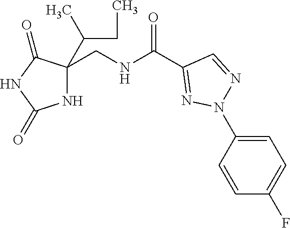 [SMILES: CCC(C)C1(CNC(=O)c2cnn(-c3ccc(F)cc3)n2)NC(=O)NC1=O]

2-(4-fluorophenyl)-2H-1,2,3-triazole-4-carboxylic acid (145 mg, 699 μmol) dissolved in 15 ml of dichloromethane was treated with N-ethyl-N-isopropylpropan-2-amine (340 μl, 2.0 mmol), 3-{[(ethylimino)methylene]amino}-N,N-dimethylpropan-1-amine hydrochloride (174 mg, 909 μmol), 1H-benzotriazol-1-ol hydrate (139 mg, 909 μmol) and diamix-5-(aminomethyl)-5-sec-butylimidazolidine-2,4-dione hydrochloride (155 mg, 699 μmol). The mixture was stirred over night at room temperature. The solvent was removed on a rotary evaporator. The residue was taken up with ethyl acetate and washed with water and saturated aqueous sodium chloride solution. The organic layer was dried over sodium sulfate, filtered, Isolute® was added and the solvent was evaporated on a rotary evaporator. The crude product was purified by column chromatography (Machine: Biotage® Isolera One; column: Biotage® SNAP Ultra 10 g; cyclohexane/ethyl acetate-gradient, 12% ethyl acetate-100% ethyl acetate; flow: 36 ml/min). Product containing samples were united and the solvents were removed on a rotary evaporator. 122 mg (100% purity, 47% yield) of the title compound were obtained.

LC-MS (Method 8): Rₜ=0.79 min; MS (ESIpos): m/z=375 [M+H]⁺

¹H-NMR (400 MHz, DMSO-d6) δ [ppm]: −0.008 (1.37), 0.008 (1.52), 0.831 (4.13), 0.850 (14.86), 0.868 (16.00), 0.883 (10.07), 0.901 (5.42), 0.945 (9.24), 0.962 (9.70), 0.986 (0.72), 1.004 (0.74), 1.013 (0.85), 1.019 (0.97), 1.031 (1.00), 1.050 (1.04), 1.065 (0.93), 1.077 (0.79), 1.084 (0.95), 1.095 (0.81), 1.102 (0.75), 1.110 (0.88), 1.129 (0.66), 1.299 (0.72), 1.306 (0.79), 1.319 (0.82), 1.325 (0.87), 1.332 (0.78), 1.339 (0.75), 1.351 (0.62), 1.364 (1.39), 1.618 (0.82), 1.636 (0.94), 1.651 (1.13), 1.668 (1.68), 1.685 (1.50), 1.695 (1.81), 1.702 (1.68), 1.712 (1.66), 1.720 (1.66), 1.729 (1.08), 1.739 (0.88), 1.746 (0.66), 2.366 (0.53), 2.410 (0.40), 2.710 (0.52), 3.585 (0.79), 3.602 (0.97), 3.610 (1.10), 3.620 (2.47), 3.626 (1.50), 3.637 (2.50), 3.645 (3.32), 3.663 (3.53), 3.675 (2.47), 3.683 (1.50), 3.691 (2.54), 3.710 (0.95), 3.725 (0.81), 7.450 (0.75), 7.458 (7.13), 7.464 (2.54), 7.480 (12.17), 7.497 (2.67), 7.502 (7.76), 7.511 (0.88), 7.752 (4.09), 7.839 (3.86), 8.063 (0.81), 8.072 (7.01), 8.077 (2.96), 8.084 (7.43), 8.089 (4.47), 8.094 (7.26), 8.101 (2.83), 8.106 (6.76), 8.115 (0.78), 8.299 (1.20), 8.314 (2.64), 8.322 (1.86), 8.331 (1.82), 8.338 (2.79), 8.354 (1.27), 8.463 (12.37), 10.661 (5.23).

### Example 103

### ent-2-(4-fluorophenyl)-N-{[(4R)-4-(5-methyl-1,3-thiazol-4-yl)-2,5-dioxoimidazolidin-4-yl]methyl}2H-1,2,3-triazole-4-carboxamide

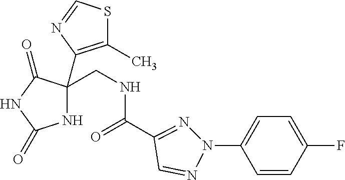 [SMILES: Cc1scnc1C1(CNC(=O)c2cnn(-c3ccc(F)cc3)n2)NC(=O)NC1=O]

Enantiomeric separation of rac-2-(4-fluorophenyl)-N-{[4-(5-methyl-1,3-thiazol-4-yl)-2,5-dioxoimidazolidin-4-yl]methyl}-2H-1,2,3-triazole-4-carboxamide (75.0 mg, 181 μmol) using the following method
Column: Diacel Chiralpak IH 5 μm 250*20 mm
Eluent A: 70% n-heptane, eluent B: 30% ethanol
Flow: 15 ml/min
UV-detection: 220 nm
Temperature: 35° C.
   afforded 35.0 mg (100% purity, 47% yield) of the desired product.

Chiral-HPLC (Column: Diacel Chiralpak IH 5 μm 250*4.6 mm; eluent A: 50% n-heptane, eluent B: 50% ethanol, flow: 1 ml/min; UV-detection: 220 nm, temperature: 50° C.): Rₜ=8.31 min; 99% ee

LC-MS (Method 7): Rₜ=1.42 min; MS (ESIpos): m/z=416 [M+H]⁺

¹H-NMR (600 MHz, DMSO-d6) δ [ppm]: 0.005 (0.48), 2.354 (16.00), 2.517 (0.99), 2.568 (0.50), 4.185 (0.51), 4.196 (0.58), 4.208 (1.42), 4.218 (1.27), 4.228 (1.32), 4.239 (1.47), 4.251 (0.61), 4.262 (0.54), 7.468 (1.94), 7.472 (0.70), 7.483 (3.06), 7.494 (0.73), 7.497 (2.09), 8.081 (1.95), 8.085 (0.83), 8.089 (2.07), 8.093 (1.25), 8.096 (2.13), 8.100 (0.81), 8.104 (1.99), 8.379 (3.30), 8.494 (7.05), 8.511 (0.74), 8.522 (1.45), 8.533 (0.72), 8.935 (7.00).

### Example 104

### ent-N-[(4-sec-butyl-2,5-dioxoimidazolidin-4-yl)methyl]-2-(4-fluorophenyl)-2H-1,2,3-triazole-4-carboxamide

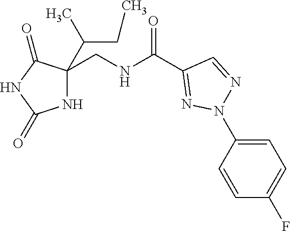 [SMILES: CCC(C)C1(CNC(=O)c2cnn(-c3ccc(F)cc3)n2)NC(=O)NC1=O]

Enantiomeric separation of diamix-N-[(4-sec-butyl-2,5-dioxoimidazolidin-4-yl)methyl]-2-(4-fluorophenyl)-2H-1,2,3-triazole-4-carboxamide (120 mg, 321 μmol) using the following method
Column: Diacel Chiralpak OZ-H 5 μm 250*20 mm
Eluent A: 70% n-heptane, eluent B: 30% ethanol
Flow: 15 ml/min
UV-detection: 220 nm
Temperature: 40° C.
   afforded 25.0 mg (100% purity, 21% yield) of the desired product.

Chiral-HPLC (Column: Diacel Chiralpak IA 5 μm 250*4.6 mm; eluent A: 0% n-heptane, eluent B: 100% ethanol, flow: 1 ml/min; UV-detection: 220 nm, temperature: 60° C.): Rₜ=5.29 min; 99% ee

LC-MS (Method 7): Rₜ=1.56 min; MS (ESIpos): m/z=375 [M+H]⁺

¹H-NMR (400 MHz, DMSO-d6) δ [ppm]: −0.008 (2.79), 0.008 (3.23), 0.831 (5.67), 0.849 (14.79), 0.868 (7.43), 0.946 (13.29), 0.963 (13.97), 1.051 (0.79), 1.069 (0.92), 1.077 (1.00), 1.084 (1.25), 1.095 (1.11), 1.102 (1.22), 1.110 (1.28), 1.129 (0.93), 1.288 (0.50), 1.300 (1.07), 1.307 (1.17), 1.319 (1.20), 1.325 (1.29), 1.333 (1.14), 1.339 (1.09), 1.351 (0.83), 1.358 (0.78), 1.696 (1.00), 1.703 (1.26), 1.713 (1.22), 1.721 (1.79), 1.729 (1.17), 1.739 (1.12), 1.746 (0.89), 3.586 (1.11), 3.603 (1.24), 3.620 (3.27), 3.637 (3.13), 3.649 (3.13), 3.665 (3.27), 3.683 (1.24), 3.699 (1.09), 7.449 (0.52), 7.458 (5.49), 7.464 (1.85), 7.480 (8.96), 7.490 (1.00), 7.496 (1.92), 7.502 (5.92), 7.511 (0.63), 7.840 (8.03), 8.063 (0.57), 8.071 (5.57), 8.077 (2.17), 8.083 (5.89), 8.089 (3.36), 8.094 (5.79), 8.100 (2.04), 8.106 (5.39), 8.115 (0.53), 8.299 (1.68), 8.315 (3.45), 8.331 (1.64), 8.463 (16.00), 10.659 (2.93).

### Example 105

### 2-(1,3-benzothiazol-2-yl)-N-{[(4R)-4-cyclopropyl-2,5-dioxoimidazolidin-4-yl]methyl}-2H-1,2,3-triazole-4-carboxamide

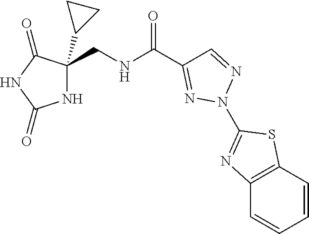 [SMILES: O=C1NC(=O)[C@](CNC(=O)c2cnn(-c3nc4ccccc4s3)n2)(C2CC2)N1]

2-(1,3-benzothiazol-2-yl)-2H-1,2,3-triazole-4-carboxylic acid (36.0 mg, 146 μmol) dissolved in 3.5 ml of dichloromethane was treated with N-ethyl-N-isopropylpropan-2-amine (71 μl, 410 μmol), 3-{[(ethylimino)methylene]amino}-N,N-dimethylpropan-1-amine hydrochloride (36.4 mg, 190 μmol), 1H-benzotriazol-1-ol hydrate (29.1 mg, 190 μmol) and (5R)-5-(aminomethyl)-5-cyclopropylimidazolidine-2,4-dione hydrochloride (30.1 mg, 146 μmol). The mixture was stirred 48 h at room temperature. The solvent was removed on a rotary evaporator. The residue was taken up with dimethylformamide and purified by preparative HPLC (Instrument: Waters Prep LC/MS System, Column: Phenomenex Kinetex C185 μm 100×30 mm; eluent A: water, eluent B: acetonitrile, eluent C: 2% formic acid in water, eluent D: acetonitrile/water (80 Vol. %/20 Vol %); flow: 80 ml/min, room temperature, UV-detection: 200-400 nm, At-Column Injektion; gradient: eluent A 0 bis 2 min 63 ml, eluent B 0 bis 2 min 7 ml, eluent A 2 bis 10 min from 63 ml to 39 ml and eluent B from 7 ml to 31 ml, 10 to 12 min 0 ml eluent A and 70 ml eluent B. Eluent C and Eluent D constant flow 5 ml/min over the whole runtime). After lyophilization, 28.0 mg (92% purity, 44% yield) of the title compound were obtained.

LC-MS (Method 7): Rₜ=1.31 min; MS (ESIpos): m/z=398 [M+H]⁺

¹H-NMR (400 MHz, DMSO-d6) δ [ppm]: −0.149 (0.43), −0.008 (3.11), 0.008 (3.54), 0.123 (0.98), 0.131 (1.47), 0.146 (2.02), 0.155 (1.53), 0.161 (1.34), 0.173 (0.73), 0.314 (0.43), 0.334 (1.47), 0.346 (1.53), 0.352 (1.77), 0.361 (1.40), 0.372 (1.16), 0.382 (0.92), 0.425 (2.63), 0.442 (5.25), 0.461 (4.27), 0.480 (1.47), 1.146 (0.67), 1.163 (1.59), 1.180 (2.20), 1.196 (1.53), 1.213 (0.55), 2.328 (0.61), 2.367 (0.92), 2.524 (2.63), 2.670 (0.55), 2.710 (0.92), 3.679 (0.43), 3.695 (0.49), 3.714 (5.07), 3.718 (4.89), 3.730 (4.76), 3.734 (4.95), 3.752 (0.55), 3.768 (0.43), 6.571 (6.60), 7.505 (0.43), 7.528 (1.95), 7.531 (2.02), 7.549 (4.15), 7.566 (3.24), 7.569 (3.18), 7.600 (2.87), 7.604 (3.11), 7.621 (4.21), 7.624 (4.03), 7.639 (7.45), 7.671 (0.55), 7.692 (0.79), 7.712 (0.55), 7.823 (1.04), 7.845 (0.73), 8.061 (4.46), 8.080 (4.09), 8.114 (0.85), 8.136 (0.79), 8.205 (4.21), 8.223 (3.85), 8.678 (16.00), 8.742 (1.71), 8.758 (3.79), 8.774 (1.77), 10.673 (5.01).

### Example 106

### N-{[(4R)-4-cyclopropyl-2,5-dioxoimidazolidin-4-yl]methyl}-2-[4-(trifluoromethoxy)phenyl]-2H-1,2,3-triazole-4-carboxamide

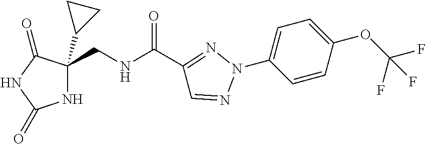 [SMILES: O=C1NC(=O)[C@](CNC(=O)c2cnn(-c3ccc(OC(F)(F)F)cc3)n2)(C2CC2)N1]

2-[4-(trifluoromethoxy)phenyl]-2H-1,2,3-triazole-4-carboxylic acid (14.7 mg, 53.6 μmol) dissolved in 2 ml of dichloromethane was treated with N-ethyl-N-isopropylpropan-2-amine (26 μl, 150 μmol 3-{[(ethylimino)methylene]amino}-N,N-dimethylpropan-1-amine hydrochloride (13.4 mg, 69.7 μmol), 1H-benzotriazol-1-ol hydrate (10.7 mg, 69.7 μmol) and (5R)-5-(aminomethyl)-5-cyclopropylimidazolidine-2,4-dione hydrochloride (11.0 mg, 53.6 μmol). The mixture was stirred over night at room temperature. The solvent was removed on a rotary evaporator. The residue was taken up with dimethylformamide and purified by preparative HPLC (Instrument: Waters Prep LC/MS System, Column: Phenomenex Kinetex C185 μm 100×30 mm; eluent A: water, eluent B: acetonitrile, eluent C: 2% formic acid in water, eluent D: acetonitrile/water (80 Vol. %/20 Vol %); flow: 80 ml/min; room temperature, UV-detection: 200-400 nm, At-Column Injektion; gradient: eluent A 0 bis 2 min 55 ml, eluent B 0 bis 2 min 15 ml, eluent A 2 bis 10 min from 55 ml to 31 ml and eluent B from 15 ml to 39 ml, 10 to 12 min 0 ml eluent A and 70 ml eluent B. Eluent C and Eluent D constant flow 5 ml/min over the whole runtime). After lyophilization, 7.00 mg (100% purity, 31% yield) of the title compound were obtained.

LC-MS (Method 7): Rₜ=1.61 min; MS (ESIpos): m/z=425 [M+H]⁺

¹H-NMR (600 MHz, DMSO-d₆) δ[ppm]: 0.118 (0.53), 0.126 (1.06), 0.133 (1.51), 0.141 (1.80), 0.144 (1.47), 0.152 (1.31), 0.160 (0.69), 0.323 (0.49), 0.337 (1.27), 0.344 (1.39), 0.348 (1.67), 0.356 (1.59), 0.362 (0.98), 0.369 (0.78), 0.429 (1.59), 0.432 (1.55), 0.443 (4.29), 0.454 (3.67), 0.467 (1.63), 1.150 (0.73), 1.159 (1.35), 1.163 (1.43), 1.173 (2.12), 1.182 (1.35), 1.195 (0.57), 2.426 (0.53), 2.521 (0.69), 2.655 (0.53), 3.296 (0.53), 3.303 (0.57), 3.349 (0.49), 3.355 (0.73), 3.361 (0.41), 3.651 (1.76), 3.661 (1.92), 3.674 (3.14), 3.684 (2.73), 3.728 (2.82), 3.739 (3.10), 3.750 (1.84), 3.762 (1.67), 6.639 (0.65), 7.635 (7.14), 7.646 (6.16), 7.660 (6.33), 8.171 (1.31), 8.176 (10.86), 8.180 (3.31), 8.188 (3.22), 8.191 (10.20), 8.197 (0.98), 8.510 (16.00), 8.524 (3.43), 8.535 (1.71), 10.662 (4.49).

### Example 107

### ent-2-(3,4-difluorophenyl)-N-{[2,5-dioxo-4-(1,3-thiazol-2-yl)imidazolidin-4-yl]methyl}-2H-1,2,3-triazole-4-carboxamide

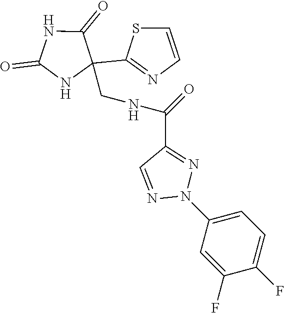 [SMILES: O=C1NC(=O)C(CNC(=O)c2cnn(-c3ccc(F)c(F)c3)n2)(c2nccs2)N1]

To a solution of rac-5-(aminomethyl)-5-(1,3-thiazol-2-yl)imidazolidine-2,4-dione hydrochloride (80.0 mg, 322 μmol) and 2-(3,4-difluorophenyl)-2H-1,2,3-triazole-4-carboxylic acid (72.4 mg, 322 μmol) in DMF (2 ml) was added 1-ethyl-3-(3-dimethylaminopropyl)carbodiimide hydrochloride (80.2 mg, 418 μmol), 1-hydroxybenzotriazole hydrate (64.0 mg, 418 μmol) and N,N-diisopropylethylamine (280 μl, 1.6 mmol). The reaction mixture was first stirred overnight at RT, then, for 8 h at 60° C. and finally overnight at RT. The reaction mixture was concentrated under reduced pressure and the crude product was first purified by preparative HPLC (Method 2f). A second purification by preparative chiral HPLC [sample preparation: 26 mg dissolved in a mixture of ethanol and acetonitrile (4/1); injection volume: 0.5 ml; column: Daicel Chiralpack IG 5 μm 250*20 mm; eluent:ethanol; flow rate: 15 ml/min; temperature: 60° C.; UV detection: 220 nm] gave 7 mg (100% purity, 5% yield) of the desired product

Analytical chiral HPLC: Rₜ=7.20 min, e.e. =>99% [column: 250×4.6 mm filled with Daicel Chiralpak IG 5 μm; eluent: 100% ethanol; flow rate: 1 ml/min; temperature: 60° C.; UV detection: 220 nm]

¹H-NMR (500 MHz, DMSO-d6) δ [ppm]: −0.120 (0.48), −0.007 (4.66), 0.006 (4.28), 0.116 (0.50), 2.363 (0.51), 2.637 (0.51), 4.066 (2.07), 4.079 (2.18), 4.093 (2.86), 4.107 (2.57), 4.227 (2.33), 4.239 (2.57), 4.254 (1.89), 4.267 (1.70), 7.709 (1.29), 7.727 (2.74), 7.747 (2.74), 7.765 (1.47), 7.817 (6.95), 7.823 (8.04), 7.891 (8.57), 7.898 (7.16), 7.915 (2.21), 7.933 (1.80), 8.105 (1.53), 8.110 (1.44), 8.119 (1.65), 8.125 (1.79), 8.133 (1.45), 8.141 (1.47), 8.147 (1.35), 8.522 (16.00), 8.732 (1.91), 8.745 (3.62), 8.757 (2.26), 11.105 (0.92).

### Example 108

### rac-N-{[2,5-dioxo-4-(1,3-thiazol-2-yl)imidazolidin-4-yl]methyl}-2-(3-fluorophenyl)-2H-1,2,3-triazole-4-carboxamide

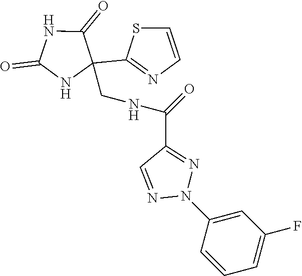 [SMILES: O=C1NC(=O)C(CNC(=O)c2cnn(-c3cccc(F)c3)n2)(c2nccs2)N1]

To a solution of rac-5-(aminomethyl)-5-(1,3-thiazol-2-yl)imidazolidine-2,4-dione hydrochloride (84.0 mg, 338 μmol) and 2-(3-fluorophenyl)-2H-1,2,3-triazole-4-carboxylic acid (70.0 mg, 338 μmol) in DMF (2.1 ml) was added 1-ethyl-3-(3-dimethylaminopropyl)carbodiimide hydrochloride (84.2 mg, 439 μmol), 1-hydroxybenzotriazole hydrate (67.3 mg, 439 μmol) and N,N-diisopropylethylamine (290 μl, 1.7 mmol). The reaction mixture was stirred overnight at RT and concentrated under reduced pressure. The crude product was purified by preparative HPLC (Method 3f). After lyophilisation, 5.00 mg (100% purity, 4% yield) of the desired product were obtained.

LC-MS (Method 8): Rₜ=0.78 min; MS (ESIpos): m/z=402 [M+H]⁺

¹H-NMR (500 MHz, DMSO-d6) δ [ppm]: 0.006 (1.47), 3.283 (0.48), 4.073 (2.37), 4.086 (2.53), 4.100 (3.26), 4.113 (2.97), 4.246 (3.08), 4.258 (3.31), 4.273 (2.57), 4.286 (2.25), 7.359 (1.63), 7.363 (1.75), 7.375 (3.24), 7.380 (3.29), 7.392 (1.75), 7.397 (1.68), 7.660 (1.82), 7.673 (2.46), 7.677 (3.61), 7.689 (3.63), 7.693 (2.14), 7.705 (1.77), 7.828 (8.23), 7.834 (9.40), 7.874 (2.37), 7.878 (3.70), 7.882 (2.62), 7.901 (11.79), 7.908 (8.21), 7.920 (4.55), 7.937 (3.72), 8.525 (16.00), 8.750 (2.25), 8.763 (4.44), 8.775 (2.14), 8.849 (9.01), 11.121 (3.43).

### Example 109

### rac-2-(4-chloro-3-fluorophenyl)-N-{[2,5-dioxo-4-(1,3-thiazol-2-yl)imidazolidin-4-yl]methyl}-2H-1,2,3-triazole-4-carboxamide

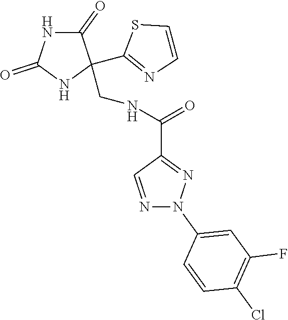 [SMILES: O=C1NC(=O)C(CNC(=O)c2cnn(-c3ccc(Cl)c(F)c3)n2)(c2nccs2)N1]

To a solution of rac-5-(aminomethyl)-5-(1,3-thiazol-2-yl)imidazolidine-2,4-dione hydrochloride (90.0 mg, 362 μmol) and 2-(4-chloro-3-fluorophenyl)-2H-1,2,3-triazole-4-carboxylic acid (87.4 mg, 362 μmol) in DMF (2.2 ml) was added 1-ethyl-3-(3-dimethylaminopropyl)carbodiimide hydrochloride (90.2 mg, 470 μmol), 1-hydroxybenzotriazole hydrate (72.0 mg, 470 μmol) and N,N-diisopropylethylamine (320 μl, 1.8 mmol). The reaction mixture was stirred overnight at RT and concentrated under reduced pressure. The crude product was purified by preparative HPLC (Method 4f). After lyophilisation, 44.5 mg (94% purity, 27% yield) of the desired product were obtained.

LC-MS (Method 12): Rₜ=2.46 min; MS (ESIpos): m/z=436 [M+H]⁺

¹H-NMR (400 MHz, DMSO-d6) δ [ppm]: 0.008 (1.11), 2.073 (12.53), 2.328 (0.82), 2.366 (0.61), 2.670 (0.77), 2.710 (0.58), 4.068 (1.98), 4.084 (2.13), 4.102 (3.03), 4.119 (2.74), 4.236 (2.79), 4.252 (3.13), 4.271 (2.27), 4.286 (1.97), 7.821 (8.05), 7.829 (10.38), 7.854 (2.72), 7.876 (5.12), 7.895 (14.21), 7.903 (8.23), 7.930 (4.45), 7.934 (4.54), 7.952 (2.32), 7.956 (2.39), 8.053 (0.47), 8.060 (0.51), 8.073 (3.84), 8.079 (4.00), 8.098 (3.80), 8.104 (3.65), 8.479 (1.29), 8.540 (16.00), 8.554 (0.58), 8.747 (1.83), 8.762 (3.86), 8.778 (1.85), 8.822 (6.42), 11.100 (5.15).

### Example 110

### ent-2-(4-chloro-3-fluorophenyl)-N-{[2,5-dioxo-4-(1,3-thiazol-2-yl)imidazolidin-4-yl]methyl}-2H-1,2,3-triazole-4-carboxamide

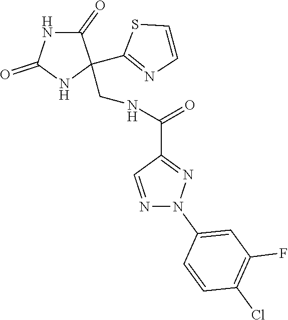 [SMILES: O=C1NC(=O)C(CNC(=O)c2cnn(-c3ccc(Cl)c(F)c3)n2)(c2nccs2)N1]

Enantiomeric separation of rac-2-(4-chloro-3-fluorophenyl)-N-{[2,5-dioxo-4-(1,3-thiazol-2-yl)imidazolidin-4-yl]methyl}-2H-1,2,3-triazole-4-carboxamide (44 mg) by preparative chiral HPLC [column: Daicel Chiralpak IC 5 μm, 250×20 mm; eluent: 70% n-heptane/30% ethanol; flow rate: 15 ml/min; temperature: 55° C.; UV detection: 220 nm] afforded 21.7 mg of the desired product (99% purity).

Analytical chiral HPLC: Rₜ=9.22 min, e.e. =>99% [column: 250×4.6 mm filled with Daicel Chiralpak IC 5 μm; eluent: 70% n-heptane/30% ethanol; flow rate: 1 ml/min; temperature: 55° C.; UV detection: 220 nm]

LC-MS (Method 7): Rₜ=1.52 min; MS (ESIpos): m/z=436 [M+H]⁺

¹H-NMR (500 MHz, DMSO-d6) δ [ppm]: 0.846 (0.64), 0.861 (0.79), 0.872 (0.60), 1.088 (0.50), 1.101 (0.52), 2.403 (0.58), 2.634 (0.39), 2.678 (0.52), 3.261 (0.52), 3.288 (1.08), 3.392 (0.94), 4.074 (2.35), 4.087 (2.51), 4.102 (3.30), 4.115 (3.03), 4.243 (3.01), 4.255 (3.35), 4.270 (2.54), 4.282 (2.29), 7.825 (8.00), 7.831 (9.41), 7.860 (2.83), 7.878 (5.42), 7.893 (4.95), 7.898 (9.79), 7.905 (8.04), 7.932 (4.30), 7.936 (4.16), 7.950 (2.51), 7.955 (2.54), 8.077 (3.78), 8.082 (3.59), 8.098 (3.78), 8.102 (3.62), 8.543 (16.00), 8.763 (2.22), 8.775 (4.51), 8.788 (2.24), 8.832 (9.10), 11.099 (1.23), 11.139 (0.54).

### Example 111

### rac-2-(3-chloro-4-fluorophenyl)-N-{[2,5-dioxo-4-(1,3-thiazol-2-yl)imidazolidin-4-yl]methyl}-2H-1,2,3-triazole-4-carboxamide

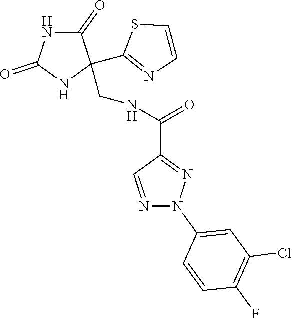 [SMILES: O=C1NC(=O)C(CNC(=O)c2cnn(-c3ccc(F)c(Cl)c3)n2)(c2nccs2)N1]

To a solution of rac-5-(aminomethyl)-5-(1,3-thiazol-2-yl)imidazolidine-2,4-dione hydrochloride (22.6 mg, 91.1 μmol) and 2-(3-chloro-4-fluorophenyl)-2H-1,2,3-triazole-4-carboxylic acid (22.0 mg, 91.1 μmol) in DMF (570 μl) was added 1-ethyl-3-(3-dimethylaminopropyl)carbodiimide hydrochloride (22.7 mg, 118 μmol), 1-hydroxybenzotriazole hydrate (18.1 mg, 118 μmol) and N,N-diisopropylethylamine (79 μl, 460 μmol). The reaction mixture was stirred overnight at RT and concentrated under reduced pressure. The crude product was purified by preparative HPLC (Method 3f). After lyophilisation, 7.00 mg (100% purity, 18% yield) of the desired product were obtained.

LC-MS (Method 8): Rₜ=0.86 min; MS (ESIpos): m/z=436 [M+H]⁺

¹H-NMR (500 MHz, DMSO-d6) δ [ppm]: 0.006 (2.72), 2.076 (0.40), 2.364 (0.55), 2.637 (0.55), 3.373 (0.51), 4.072 (2.28), 4.085 (2.46), 4.100 (3.16), 4.113 (2.79), 4.239 (2.94), 4.251 (3.19), 4.266 (2.46), 4.279 (2.13), 7.691 (3.30), 7.709 (6.68), 7.727 (3.52), 7.826 (8.37), 7.833 (9.43), 7.898 (9.76), 7.904 (7.89), 8.054 (2.24), 8.059 (2.79), 8.062 (2.79), 8.067 (2.50), 8.072 (2.31), 8.077 (2.61), 8.080 (2.39), 8.086 (1.94), 8.251 (3.78), 8.256 (3.67), 8.264 (3.78), 8.269 (3.23), 8.525 (16.00), 8.774 (2.24), 8.786 (4.26), 8.799 (2.09), 8.835 (8.37), 11.115 (4.00).

### Example 112

### ent-2-(3-chloro-4-fluorophenyl)-N-{[2,5-dioxo-4-(1,3-thiazol-2-yl)imidazolidin-4-yl]methyl}-2H-1,2,3-triazole-4-carboxamide

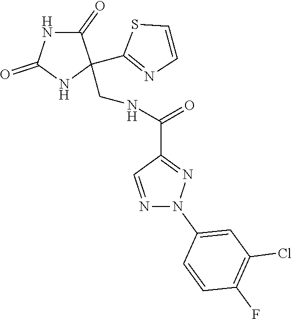 [SMILES: O=C1NC(=O)C(CNC(=O)c2cnn(-c3ccc(F)c(Cl)c3)n2)(c2nccs2)N1]

Enantiomeric separation of rac-2-(3-chloro-4-fluorophenyl)-N-{[2,5-dioxo-4-(1,3-thiazol-2-yl)imidazolidin-4-yl]methyl}-2H-1,2,3-triazole-4-carboxamide (6 mg) by preparative chiral HPLC [column: Daicel Chiralpak IC 5 μm, 250×20 mm; eluent: 60% n-heptane/40% ethanol; flow rate: 20 ml/min; temperature: 45° C.; UV detection: 210 nm] afforded 1.73 mg of the desired product (98% purity).

Analytical chiral HPLC: Rₜ=1.84 min, e.e. =>99% [column: Daicel Chiralpak IC-3 3 μm, 50×4.6 mm; eluent: 50% n-heptane/50% ethanol; flow rate: 1 ml/min; UV detection: 220 nm]

LC-MS (Method 7): Rₜ=1.49 min; MS (ESIpos): m/z=436 [M+H]⁺

¹H-NMR (500 MHz, DMSO-d6) δ [ppm]: 0.858 (1.22), 1.031 (0.46), 1.045 (0.46), 1.141 (0.86), 1.154 (1.42), 1.169 (0.86), 1.235 (2.69), 2.365 (1.07), 2.639 (0.97), 2.911 (0.66), 2.925 (0.66), 4.072 (3.30), 4.085 (3.66), 4.100 (4.67), 4.113 (4.32), 4.240 (4.42), 4.252 (4.83), 4.267 (3.71), 4.280 (3.25), 7.692 (3.96), 7.710 (8.03), 7.728 (4.42), 7.827 (8.84), 7.833 (10.57), 7.898 (10.16), 7.904 (9.45), 8.062 (4.77), 8.080 (4.52), 8.257 (5.64), 8.265 (5.64), 8.526 (16.00), 8.780 (3.56), 8.792 (6.86), 8.804 (3.96), 8.840 (11.17), 11.098 (0.81).

### Example 113

### rac-N-{[2,5-dioxo-4-(1,3-thiazol-2-yl)imidazolidin-4-yl]methyl}-2-(4-fluorophenyl)-2H-1,2,3-triazole-4-carboxamide

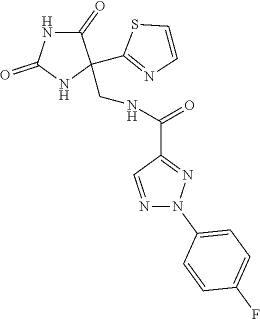 [SMILES: O=C1NC(=O)C(CNC(=O)c2cnn(-c3ccc(F)cc3)n2)(c2nccs2)N1]

To a solution of rac-5-(aminomethyl)-5-(1,3-thiazol-2-yl)imidazolidine-2,4-dione hydrochloride (90.0 mg, 362 μmol) and 2-(4-fluorophenyl)-2H-1,2,3-triazole-4-carboxylic acid (75.0 mg, 362 μmol) in DMF (2.2 ml) was added 1-ethyl-3-(3-dimethylaminopropyl)carbodiimide hydrochloride (90.2 mg, 470 μmol), 1-hydroxybenzotriazole hydrate (72.0 mg, 470 μmol) and N,N-diisopropylethylamine (320 μl, 1.8 mmol). The reaction mixture was stirred overnight at RT and concentrated under reduced pressure. The crude product was purified by preparative HPLC (Method 3f). After lyophilisation, 50.7 mg (100% purity, 35% yield) of the desired product were obtained.

LC-MS (Method 8): Rₜ=0.78 min; MS (ESIpos): m/z=402 [M+H]⁺

¹H-NMR (400 MHz, DMSO-d6) δ [ppm]: −0.149 (0.46), −0.008 (3.72), 0.008 (3.56), 0.146 (0.41), 2.328 (0.45), 2.367 (0.48), 2.524 (1.58), 2.671 (0.50), 2.711 (0.50), 4.055 (2.34), 4.071 (2.55), 4.089 (3.39), 4.106 (3.12), 4.245 (3.12), 4.260 (3.47), 4.279 (2.58), 4.295 (2.30), 7.429 (0.47), 7.437 (4.43), 7.443 (1.58), 7.450 (1.35), 7.459 (12.31), 7.464 (4.14), 7.481 (14.23), 7.490 (1.64), 7.497 (2.29), 7.503 (6.56), 7.511 (0.70), 7.820 (8.18), 7.828 (10.07), 7.895 (10.28), 7.903 (8.36), 8.065 (1.08), 8.074 (9.61), 8.079 (4.00), 8.086 (10.16), 8.091 (6.03), 8.097 (9.79), 8.103 (3.77), 8.109 (9.23), 8.117 (0.99), 8.480 (16.00), 8.490 (5.46), 8.666 (2.00), 8.682 (4.29), 8.698 (2.01), 8.822 (8.40), 11.100 (3.30).

### Example 114

### ent-N-{[2,5-dioxo-4-(1,3-thiazol-2-yl)imidazolidin-4-yl]methyl}-2-(4-fluorophenyl)-2H-1,2,3-triazole-4-carboxamide

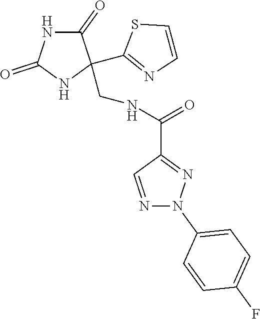 [SMILES: O=C1NC(=O)C(CNC(=O)c2cnn(-c3ccc(F)cc3)n2)(c2nccs2)N1]

Enantiomeric separation of rac-N-{[2,5-dioxo-4-(1,3-thiazol-2-yl)imidazolidin-4-yl]methyl}-2-(4-fluorophenyl)-2H-1,2,3-triazole-4-carboxamide (50.4 mg) by preparative chiral HPLC [column: Daicel Chiralpak IC 5 μm, 250×20 mm; eluent: 60% n-heptane/40% isopropanol; flow rate: 20 ml/min; temperature: 50° C.; UV detection: 210 nm] afforded 15.8 mg of the desired product (98% purity).

Analytical chiral HPLC: Rₜ=3.05 min, e.e. =>99% [column: Daicel Chiralpak IC-3 3 μm, 50×4.6 mm; eluent: 50% n-heptane/50% isopropanol; flow rate: 1 ml/min; UV detection: 220 nm]

LC-MS (Method 7): Rₜ=1.28 min; MS (ESIpos): m/z=402 [M+H]⁺

¹H-NMR (400 MHz, DMSO-d6) δ [ppm]: −0.008 (0.93), 0.008 (0.93), 1.139 (0.57), 1.157 (1.24), 1.175 (0.63), 1.234 (0.55), 1.283 (1.40), 2.328 (0.57), 2.366 (0.73), 2.524 (2.90), 2.670 (0.61), 2.710 (0.73), 2.917 (0.49), 2.934 (0.47), 4.054 (2.07), 4.071 (2.25), 4.089 (2.98), 4.105 (2.76), 4.244 (2.68), 4.259 (3.04), 4.278 (2.27), 4.293 (2.05), 7.459 (5.45), 7.464 (1.99), 7.481 (9.00), 7.497 (2.03), 7.503 (5.86), 7.820 (8.05), 7.828 (9.86), 7.894 (9.75), 7.903 (8.01), 8.074 (5.66), 8.080 (2.39), 8.086 (5.96), 8.092 (3.59), 8.097 (5.82), 8.103 (2.23), 8.109 (5.39), 8.479 (16.00), 8.661 (1.82), 8.677 (3.83), 8.693 (1.78), 8.815 (8.03), 10.989 (0.45), 11.096 (0.87).

### Example 115

### rac-2-(2,4-difluorophenyl)-N-{[2,5-dioxo-4-(1,3-thiazol-2-yl)imidazolidin-4-yl]methyl}-2H-1,2,3-triazole-4-carboxamide

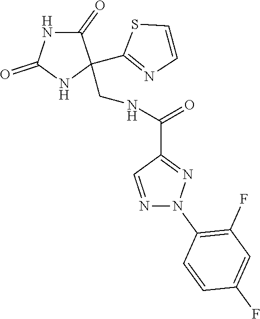 [SMILES: O=C1NC(=O)C(CNC(=O)c2cnn(-c3ccc(F)cc3F)n2)(c2nccs2)N1]

To a solution of rac-5-(aminomethyl)-5-(1,3-thiazol-2-yl)imidazolidine-2,4-dione hydrochloride (44.2 mg, 178 μmol) and 2-(2,4-difluorophenyl)-2H-1,2,3-triazole-4-carboxylic acid (40.0 mg, 178 μmol) in DMF (1.1 ml) was added 1-ethyl-3-(3-dimethylaminopropyl)carbodiimide hydrochloride (44.3 mg, 231 μmol), 1-hydroxybenzotriazole hydrate (35.4 mg, 231 μmol) and N,N-diisopropylethylamine (150 μl, 890 μmol). The reaction mixture was stirred overnight at RT and concentrated under reduced pressure. The crude product was purified by preparative HPLC (Method 3f). After lyophilisation, 15.0 mg (96% purity, 19% yield) of the desired product were obtained.

LC-MS (Method 8): Rₜ=0.74 min; MS (ESIpos): m/z=420 [M+H]⁺

¹H-NMR (400 MHz, DMSO-d6) δ [ppm]: −0.008 (2.57), 0.008 (2.41), 4.027 (2.46), 4.042 (2.67), 4.061 (3.41), 4.077 (3.10), 4.255 (3.12), 4.271 (3.45), 4.289 (2.69), 4.305 (2.42), 7.346 (1.28), 7.349 (1.42), 7.352 (1.49), 7.356 (1.37), 7.369 (2.64), 7.372 (2.81), 7.375 (2.61), 7.389 (1.38), 7.392 (1.49), 7.395 (1.55), 7.399 (1.36), 7.656 (1.93), 7.663 (1.90), 7.679 (2.19), 7.684 (3.21), 7.690 (2.05), 7.706 (1.99), 7.713 (1.83), 7.814 (7.93), 7.822 (9.94), 7.884 (9.99), 7.892 (8.11), 7.925 (1.98), 7.940 (2.20), 7.947 (3.59), 7.962 (3.59), 7.969 (1.96), 7.984 (1.79), 8.527 (16.00), 8.639 (2.10), 8.654 (4.41), 8.670 (2.04), 8.801 (9.27), 11.091 (2.70).

### Example 116

### ent-2-(2,4-difluorophenyl)-N-{[2,5-dioxo-4-(1,3-thiazol-2-yl)imidazolidin-4-yl]methyl}-2H-1,2,3-triazole-4-carboxamide

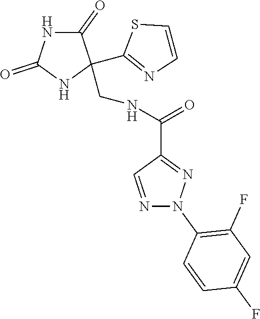 [SMILES: O=C1NC(=O)C(CNC(=O)c2cnn(-c3ccc(F)cc3F)n2)(c2nccs2)N1]

Enantiomeric separation of rac-2-(2,4-difluorophenyl)-N-{[2,5-dioxo-4-(1,3-thiazol-2-yl)imidazolidin-4-yl]methyl}-2H-1,2,3-triazole-4-carboxamide (14 mg) by preparative chiral HPLC [column: Daicel Chiralpak IE 5 μm, 250×20 mm; eluent: 50% n-heptane/50% ethanol; flow rate: 20 ml/min; temperature: 45° C.; UV detection: 210 nm] afforded 1.98 mg of the desired product (98% purity).

Analytical chiral HPLC: Rₜ=3.74 min, e.e. =>99% [column: Daicel Chiralpak IE-3 3 μm, 50×4.6 mm; eluent: 50% n-heptane/50% ethanol; flow rate: 1 ml/min; UV detection: 220 nm]

LC-MS (Method 7): Rₜ=1.23 min; MS (ESIpos): m/z=420 [M+H]⁺

¹H-NMR (400 MHz, DMSO-d6) δ [ppm]: 1.137 (0.48), 1.156 (0.88), 1.174 (0.49), 1.235 (0.62), 2.328 (0.83), 2.367 (0.46), 2.670 (0.96), 2.711 (0.51), 4.026 (2.34), 4.042 (2.50), 4.060 (3.22), 4.076 (2.93), 4.254 (2.96), 4.270 (3.25), 4.289 (2.52), 4.304 (2.31), 7.352 (1.45), 7.372 (2.88), 7.395 (1.59), 7.656 (1.83), 7.663 (1.83), 7.679 (2.18), 7.684 (3.30), 7.690 (2.13), 7.706 (1.94), 7.713 (1.81), 7.813 (7.72), 7.821 (9.58), 7.883 (9.63), 7.892 (7.83), 7.925 (1.89), 7.939 (2.15), 7.947 (3.47), 7.961 (3.55), 7.969 (1.97), 7.983 (1.75), 8.527 (16.00), 8.638 (2.01), 8.653 (4.22), 8.668 (2.01), 8.799 (8.31), 11.084 (0.60).

### Example 117

### ent-N-[(4-isopropyl-2,5-dioxoimidazolidin-4-yl)methyl]-2-(3,4,5-trifluorophenyl)-2H-1,2,3-triazole-4-carboxamide

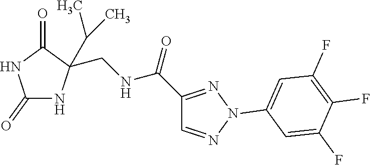 [SMILES: CC(C)C1(CNC(=O)c2cnn(-c3cc(F)c(F)c(F)c3)n2)NC(=O)NC1=O]

Enantiomeric separation of rac-N-[(4-isopropyl-2,5-dioxoimidazolidin-4-yl)methyl]-2-(3,4,5-trifluorophenyl)-2H-1,2,3-triazole-4-carboxamide (51 mg) by preparative chiral HPLC [column: Daicel Chiralcel® OX-H 5 μm, 250×20 mm; eluent: 60% n-heptane/40% isopropanol; flow rate: 15 ml/min; temperature: 40° C.; UV detection: 210 nm] afforded 23.2 mg of the desired product (98% purity).

Analytical chiral HPLC: Rₜ=1.36 min, e.e. =>99% [column: 50×4.6 mm filled with Daicel Chiralpak OX-3 3 μm; eluent: 50% n-heptane/50% isopropanol; flow rate: 1 ml/min; UV detection: 220 nm

¹H NMR (500 MHz, DMSO-d₆) b ppm 10.66 (bs, 1H), 8.41-8.63 (m, 2H), 8.03 (dd, 2H), 7.82 (bs, 1H), 3.46-3.79 (m, 2H), 1.92-2.10 (m, 1H), 0.77-1.02 (m, 6H)

### Example 118

### rac-2-(3-fluorophenyl)-N-[(4-isopropyl-2,5-dioxoimidazolidin-4-yl)methyl]-2H-1,2,3-triazole-4-carboxamide

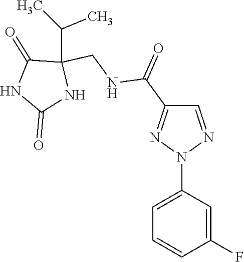 [SMILES: CC(C)C1(CNC(=O)c2cnn(-c3cccc(F)c3)n2)NC(=O)NC1=O]

To a solution of rac-5-(aminomethyl)-5-(propan-2-yl)imidazolidine-2,4-dione (80.0 mg, 467 μmol) and 2-(3-fluorophenyl)-2H-1,2,3-triazole-4-carboxylic acid (96.8 mg, 467 μmol) in DMF (9.6 ml) was added 1-ethyl-3-(3-dimethylaminopropyl)carbodiimide hydrochloride (116 mg, 607 μmol), 1-hydroxybenzotriazole hydrate (93.0 mg, 607 μmol) and N,N-diisopropylethylamine (230 μl, 1.3 mmol). After stirring overnight at RT, extra portion of N,N-diisopropylethylamine (23 μl, 130 μmol) was added and the reaction mixture was stirred for 1 h at 60° C. and concentrated under reduced pressure. The crude product was first purified by preparative HPLC (Method 2f). A second purification by preparative HPLC (Method 3f) gave 56.4 mg (98% purity, 33% yield) of the desired product

LC-MS (Method 11): Rₜ=0.99 min; MS (ESIpos): m/z=361 [M+H]⁺

¹H-NMR (400 MHz, DMSO-d6) δ [ppm]: 0.845 (15.54), 0.862 (16.00), 0.955 (15.29), 0.972 (15.87), 1.970 (0.91), 1.986 (2.38), 2.004 (3.17), 2.020 (2.23), 2.038 (0.80), 2.073 (1.01), 2.328 (0.56), 2.670 (0.56), 3.573 (1.63), 3.589 (1.78), 3.607 (3.58), 3.624 (3.32), 3.657 (3.28), 3.673 (3.48), 3.691 (1.67), 3.707 (1.57), 7.349 (1.31), 7.354 (1.43), 7.370 (2.78), 7.375 (2.99), 7.391 (1.55), 7.396 (1.65), 7.652 (1.78), 7.668 (2.17), 7.673 (3.65), 7.688 (3.63), 7.693 (2.23), 7.709 (1.84), 7.805 (6.55), 7.863 (1.79), 7.868 (3.09), 7.874 (2.09), 7.887 (1.80), 7.893 (3.10), 7.898 (2.21), 7.914 (4.00), 7.935 (3.22), 7.938 (2.93), 8.387 (1.90), 8.403 (3.92), 8.419 (1.88), 8.500 (13.37), 10.656 (5.58).

### Example 119

### ent-2-(3-fluorophenyl)-N-[(4-isopropyl-2,5-dioxoimidazolidin-4-yl)methyl]-2H-1,2,3-triazole-4-carboxamide

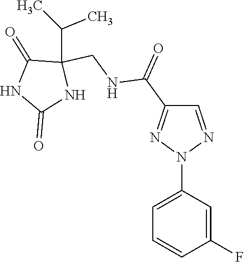 [SMILES: CC(C)C1(CNC(=O)c2cnn(-c3cccc(F)c3)n2)NC(=O)NC1=O]

Enantiomeric separation of rac-2-(3-fluorophenyl)-N-[(4-isopropyl-2,5-dioxoimidazolidin-4-yl)methyl]-2H-1,2,3-triazole-4-carboxamide (42.4 mg) by preparative chiral HPLC [column: Daicel Chiralpak IC 5 μm, 250×20 mm; eluent: 80% n-heptan/20% ethanol; flow rate: 20 ml/min; temperature: 40° C.; UV detection: 220 nm] afforded 5.4 mg of the desired product (99% purity).

Analytical chiral HPLC: Rₜ=8.46 min, e.e. =>99% [column: 250×4.6 mm filled with Daicel Chiralpak IC 5 μm; eluent: 80% n-heptane/20% ethanol; flow rate: 1 ml/min; temperature: 40° C.; UV detection: 220 nm]

¹H-NMR (600 MHz, DMSO-d6) δ [ppm]: −0.189 (0.52), 0.659 (4.24), 0.670 (4.37), 0.771 (4.19), 0.782 (4.26), 1.805 (0.71), 1.816 (0.90), 1.827 (0.67), 2.318 (5.89), 3.163 (16.00), 3.389 (0.57), 3.399 (0.63), 3.411 (0.92), 3.423 (0.83), 3.484 (0.85), 3.495 (0.91), 3.507 (0.60), 3.518 (0.56), 7.177 (0.53), 7.191 (1.01), 7.202 (0.55), 7.477 (0.44), 7.490 (0.97), 7.500 (0.98), 7.513 (0.44), 7.658 (2.49), 7.690 (1.00), 7.706 (0.99), 7.733 (1.24), 7.747 (1.12), 8.255 (0.65), 8.266 (1.23), 8.276 (0.64), 8.326 (3.34), 10.486 (1.00).

### Example 120

### rac-2-(3-cyanophenyl)-N-[(4-isopropyl-2,5-dioxoimidazolidin-4-yl)methyl]-2H-1,2,3-triazole-4-carboxamide

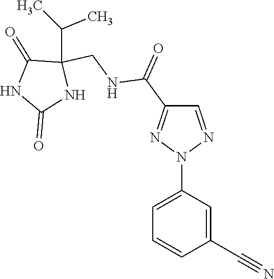 [SMILES: CC(C)C1(CNC(=O)c2cnn(-c3cccc(C#N)c3)n2)NC(=O)NC1=O]

To a solution of rac-5-(aminomethyl)-5-(propan-2-yl)imidazolidine-2,4-dione (80.0 mg, 467 μmol) and 2-(3-cyanophenyl)-2H-1,2,3-triazole-4-carboxylic acid (100 mg, 467 μmol) in DMF (9.6 ml) was added 1-ethyl-3-(3-dimethylaminopropyl)carbodiimide hydrochloride (116 mg, 607 μmol), 1-hydroxybenzotriazole hydrate (93.0 mg, 607 μmol) and N,N-diisopropylethylamine (230 μl, 1.3 mmol). After stirring overnight at RT, extra portion of N,N-diisopropylethylamine (23 μl, 130 μmol) was added and the reaction mixture was stirred for 1 h at 60° C. and concentrated under reduced pressure. The crude product was first purified by preparative HPLC (Method 2f). A second purification by preparative HPLC (Method 3f) gave 63.5 mg (97% purity, 27.4% yield) of the desired product

LC-MS (Method 11): Rₜ=0.93 min; MS (ESIpos): m/z=368 [M+H]⁺

¹H-NMR (500 MHz, DMSO-d6) δ [ppm]: 0.847 (15.77), 0.860 (16.00), 0.961 (15.61), 0.974 (15.97), 1.222 (0.71), 1.235 (0.87), 1.755 (1.06), 1.978 (0.97), 1.992 (2.42), 2.006 (3.23), 2.019 (2.29), 2.033 (0.84), 2.364 (0.48), 2.638 (0.61), 3.563 (2.23), 3.576 (2.45), 3.591 (3.45), 3.604 (3.13), 3.684 (3.10), 3.696 (3.32), 3.711 (2.26), 3.724 (2.13), 7.830 (10.52), 7.847 (7.03), 7.863 (4.39), 7.983 (5.19), 7.998 (4.23), 8.374 (3.71), 8.378 (3.84), 8.391 (3.29), 8.393 (3.42), 8.427 (0.39), 8.478 (6.23), 8.482 (8.16), 8.486 (7.03), 8.501 (2.13), 8.548 (16.00), 8.559 (0.48), 10.670 (4.87).

### Example 121

### ent-2-(3-cyanophenyl)-N-[(4-isopropyl-2,5-dioxoimidazolidin-4-yl)methyl]-2H-1,2,3-triazole-4-carboxamide

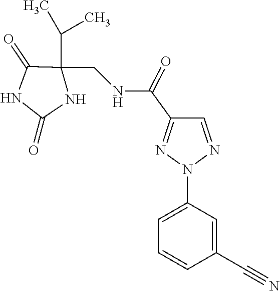 [SMILES: CC(C)C1(CNC(=O)c2cnn(-c3cccc(C#N)c3)n2)NC(=O)NC1=O]

Enantiomeric separation of rac-2-(3-cyanophenyl)-N-[(4-isopropyl-2,5-dioxoimidazolidin-4-yl)methyl]-2H-1,2,3-triazole-4-carboxamide (51 mg) by preparative chiral HPLC [column: Daicel Chiralpak OX-H 5 μm, 250×20 mm; eluent: 50% n-heptane/50% ethanol; flow rate: 20 ml/min; temperature: 40° C.; UV detection: 220 nm] afforded 23.5 mg of the desired product (98.5% purity).

Analytical chiral HPLC: Rₜ=2.43 min, e.e. =>99% [column: Daicel OX-3 3 μm, 50×4.6 mm; eluent: 50% n-heptane/50% ethanol; flow rate: 1 ml/min; UV detection: 220 nm]

LC-MS (Method 7): Rₜ=1.26 min; MS (ESIpos): m/z=368 [M+H]⁺

¹H-NMR (600 MHz, DMSO-d6) δ [ppm]: 0.850 (16.00), 0.861 (15.92), 0.962 (15.79), 0.974 (15.66), 1.112 (0.54), 1.124 (0.54), 1.985 (1.01), 1.996 (2.47), 2.007 (3.18), 2.018 (2.26), 2.030 (0.84), 2.162 (0.46), 2.426 (0.46), 2.655 (0.42), 3.300 (2.05), 3.360 (1.34), 3.573 (2.18), 3.583 (2.39), 3.596 (3.31), 3.606 (2.89), 3.684 (3.06), 3.695 (3.23), 3.707 (2.26), 3.718 (2.09), 7.815 (8.88), 7.833 (3.52), 7.846 (6.58), 7.859 (4.10), 7.981 (4.73), 7.994 (3.94), 8.375 (3.27), 8.378 (3.31), 8.391 (3.10), 8.460 (2.26), 8.471 (4.77), 8.475 (5.57), 8.478 (7.50), 8.481 (5.95), 8.543 (15.66), 10.661 (3.60).

### Example 122

### rac-2-(3,4-difluorophenyl)-N-[(4-isopropyl-2,5-dioxoimidazolidin-4-yl)methyl]-2H-1,2,3-triazole-4-carboxamide

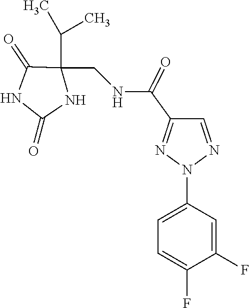 [SMILES: CC(C)C1(CNC(=O)c2cnn(-c3ccc(F)c(F)c3)n2)NC(=O)NC1=O]

To a solution of rac-5-(aminomethyl)-5-(propan-2-yl)imidazolidine-2,4-dione (100 mg, 584 μmol) and 2-(3,4-difluorophenyl)-2H-1,2,3-triazole-4-carboxylic acid (132 mg, 584 μmol) in DMF (12 ml) was added 1-ethyl-3-(3-dimethylaminopropyl)carbodiimide hydrochloride (146 mg, 759 μmol), 1-hydroxybenzotriazole hydrate (116 mg, 759 μmol) and N,N-diisopropylethylamine (280 μl, 1.6 mmol). The reaction mixture was stirred overnight, then treated with water and extracted with ethyl acetate. The combined organic phases were concentrated under reduced pressure and the crude product was purified by preparative HPLC (Column: Chromatorex C18 10 μm; 125×40 mm, Eluent A: water+0.05% trifluoroacetic acid, Eluent B: acetonitrile+0.05% trifluoroacetic acid; Gradient: 15% B→70% B) After lyophilisation, 52.2 mg (97% purity, 23% yield) of the desired product were obtained.

LC-MS (Method 11): Rₜ=1.03 min; MS (ESIpos): m/z=379 [M+H]⁺

¹H-NMR (400 MHz, DMSO-d6) δ [ppm]: 0.008 (0.52), 0.843 (15.48), 0.860 (16.00), 0.953 (15.26), 0.970 (15.85), 1.968 (0.87), 1.984 (2.36), 2.001 (3.13), 2.018 (2.23), 2.035 (0.79), 2.328 (0.69), 2.367 (0.47), 2.524 (2.24), 2.670 (0.70), 2.675 (0.52), 2.710 (0.51), 3.563 (1.69), 3.580 (1.88), 3.598 (3.41), 3.614 (3.11), 3.659 (3.05), 3.675 (3.29), 3.694 (1.77), 3.710 (1.67), 7.697 (1.41), 7.720 (3.08), 7.745 (3.14), 7.767 (1.90), 7.803 (6.14), 7.910 (2.36), 7.920 (1.60), 7.929 (1.54), 7.932 (1.93), 7.942 (1.02), 8.094 (1.52), 8.100 (1.49), 8.111 (1.70), 8.118 (1.82), 8.122 (1.87), 8.129 (1.64), 8.140 (1.65), 8.146 (1.54), 8.385 (1.80), 8.401 (3.82), 8.417 (1.80), 8.500 (14.10), 10.652 (5.36).

### Example 123

### ent-2-(3,4-difluorophenyl)-N-[(4-isopropyl-2,5-dioxoimidazolidin-4-yl)methyl]-2H-1,2,3-triazole-4-carboxamide

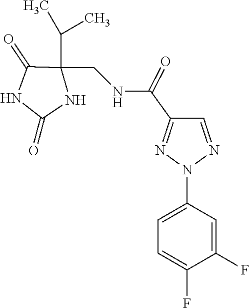 [SMILES: CC(C)C1(CNC(=O)c2cnn(-c3ccc(F)c(F)c3)n2)NC(=O)NC1=O]

Enantiomeric separation of rac-2-(3,4-difluorophenyl)-N-[(4-isopropyl-2,5-dioxoimidazolidin-4-yl)methyl]-2H-1,2,3-triazole-4-carboxamide (50.4 mg) by preparative chiral HPLC [column: Daicel Chiralpak IC 5 μm, 250×20 mm; eluent: 80% n-heptan/20% ethanol; flow rate: 20 ml/min; temperature: 40° C.; UV detection: 220 nm] afforded 4.3 mg of the desired product (99% purity).

Analytical chiral HPLC: Rₜ=8.47 min, e.e. =>99% [column: 250×4.6 mm filled with Daicel Chiralpak IC 5 μm; eluent: 80% n-heptan/20% ethanol; flow rate: 1 ml/min; temperature: 40° C.; UV detection: 220 nm]

LC-MS (Method 8): Rₜ=0.80 min; MS (ESIpos): m/z=379 [M+H]⁺

¹H-NMR (400 MHz, DMSO-d6) δ [ppm]: −0.008 (1.94), 0.008 (2.25), 0.843 (15.28), 0.860 (16.00), 0.953 (14.75), 0.970 (15.38), 1.235 (0.62), 1.967 (0.86), 1.984 (2.37), 2.001 (3.11), 2.018 (2.16), 2.035 (0.77), 2.327 (0.62), 2.366 (0.93), 2.669 (0.74), 2.710 (0.93), 3.563 (1.68), 3.579 (1.84), 3.597 (3.35), 3.614 (3.04), 3.659 (3.09), 3.675 (3.31), 3.693 (1.77), 3.709 (1.72), 7.697 (1.39), 7.719 (3.16), 7.745 (3.23), 7.767 (1.89), 7.802 (8.53), 7.909 (2.40), 7.920 (1.77), 7.932 (2.06), 8.094 (1.60), 8.100 (1.60), 8.111 (1.87), 8.122 (1.99), 8.128 (1.77), 8.139 (1.80), 8.146 (1.65), 8.385 (1.82), 8.401 (3.86), 8.417 (1.84), 8.500 (15.86), 10.641 (2.20).

### Example 124

### rac-2-(4-chloro-3-fluorophenyl)-N-[(4-isopropyl-2,5-dioxoimidazolidin-4-yl)methyl]-2H-1,2,3-triazole-4-carboxamide

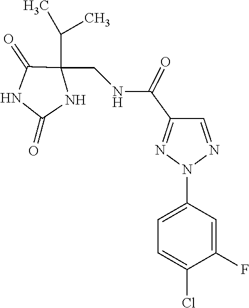 [SMILES: CC(C)C1(CNC(=O)c2cnn(-c3ccc(Cl)c(F)c3)n2)NC(=O)NC1=O]

To a solution of rac-5-(aminomethyl)-5-(propan-2-yl)imidazolidine-2,4-dione (100 mg, 584 μmol) and 2-(4-chloro-3-fluorophenyl)-2H-1,2,3-triazole-4-carboxylic acid (141 mg, 584 μmol) in DMF (12 ml) was added 1-ethyl-3-(3-dimethylaminopropyl)carbodiimide hydrochloride (146 mg, 759 μmol), 1-hydroxybenzotriazole hydrate (116 mg, 759 μmol) and N,N-diisopropylethylamine (280 μl, 1.6 mmol). The reaction mixture was stirred overnight, then treated with water and extracted with ethyl acetate. The combined organic phases were concentrated under reduced pressure and the crude product was purified by preparative HPLC (Column: Chromatorex C18 10 μm; 125×40 mm, Eluent A: water+0.05% trifluoroacetic acid, Eluent B: acetonitrile+0.05% trifluoroacetic acid; Gradient: 15% B→70% B) After lyophilisation, 35.5 mg (100% purity, 15% yield) of the desired product were obtained.

LC-MS (Method 7): Rₜ=1.55 min; MS (ESIpos): m/z=395 [M+H]⁺

¹H-NMR (400 MHz, DMSO-d6) δ [ppm]: 0.008 (1.05), 0.844 (15.52), 0.861 (16.00), 0.954 (15.22), 0.971 (15.82), 1.968 (0.90), 1.985 (2.33), 2.002 (3.14), 2.019 (2.21), 2.036 (0.81), 2.328 (0.81), 2.366 (0.57), 2.665 (0.66), 2.670 (0.84), 2.710 (0.57), 3.562 (1.76), 3.578 (1.94), 3.596 (3.41), 3.612 (3.11), 3.663 (3.05), 3.678 (3.29), 3.697 (1.85), 3.713 (1.70), 7.803 (6.43), 7.854 (2.54), 7.876 (5.05), 7.895 (4.73), 7.928 (4.13), 7.933 (4.01), 7.950 (2.15), 7.956 (2.21), 8.071 (3.56), 8.077 (3.32), 8.096 (3.56), 8.102 (3.32), 8.420 (1.88), 8.435 (4.04), 8.451 (1.91), 8.523 (13.79), 10.653 (5.62).

### Example 125

### ent-2-(4-chloro-3-fluorophenyl)-N-[(4-isopropyl-2,5-dioxoimidazolidin-4-yl)methyl]-2H-1,2,3-triazole-4-carboxamide

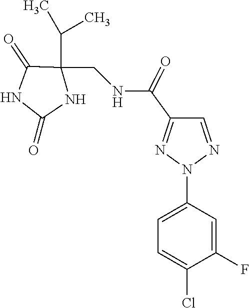 [SMILES: CC(C)C1(CNC(=O)c2cnn(-c3ccc(Cl)c(F)c3)n2)NC(=O)NC1=O]

Enantiomeric separation of rac-2-(4-chloro-3-fluorophenyl)-N-[(4-isopropyl-2,5-dioxoimidazolidin-4-yl)methyl]-2H-1,2,3-triazole-4-carboxamide (33.7 mg) by preparative chiral HPLC [column: Daicel Chiralpak IC 5 μm, 250×20 mm; eluent: 75% n-heptan/25% ethanol; flow rate: 20 ml/min; temperature: 40° C.; UV detection: 220 nm] afforded 3.9 mg of the desired product (98% purity).

Analytical chiral HPLC: Rₜ=7.25 min, e.e. =>99% [column: 250×4.6 mm filled with Daicel Chiralpak IC 5 μm; eluent: 75% n-heptan/25% ethanol; flow rate: 1 ml/min; temperature: 40° C.; UV detection: 220 nm]

¹H-NMR (400 MHz, DMSO-d6) δ [ppm]: −0.150 (0.48), −0.008 (4.14), 0.008 (4.47), 0.146 (0.44), 0.844 (13.62), 0.861 (14.24), 0.954 (13.00), 0.971 (13.51), 1.028 (0.44), 1.045 (0.40), 1.135 (0.62), 1.154 (1.17), 1.172 (0.70), 1.235 (1.32), 1.968 (0.81), 1.985 (2.05), 2.002 (2.71), 2.019 (1.94), 2.036 (0.70), 2.328 (0.92), 2.366 (1.28), 2.670 (0.99), 2.710 (1.32), 3.561 (1.50), 3.578 (1.68), 3.596 (2.89), 3.612 (2.64), 3.662 (2.60), 3.678 (2.86), 3.696 (1.65), 3.712 (1.50), 7.802 (7.18), 7.854 (2.49), 7.876 (4.76), 7.895 (4.54), 7.928 (3.73), 7.933 (3.81), 7.950 (1.94), 7.957 (2.01), 8.072 (3.59), 8.078 (3.33), 8.097 (3.55), 8.103 (3.33), 8.419 (1.61), 8.435 (3.41), 8.451 (1.65), 8.523 (16.00), 10.650 (2.53).

### Example 126

### ent-2-(3-chloro-4-fluorophenyl)-N-[(4-isopropyl-2,5-dioxoimidazolidin-4-yl)methyl]-2H-1,2,3-triazole-4-carboxamide

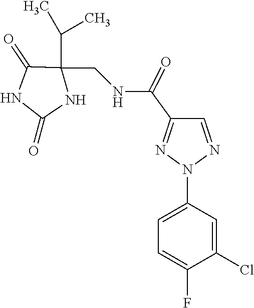 [SMILES: CC(C)C1(CNC(=O)c2cnn(-c3ccc(F)c(Cl)c3)n2)NC(=O)NC1=O]

To a solution of rac-5-(aminomethyl)-5-(propan-2-yl)imidazolidine-2,4-dione (100 mg, 584 μmol) and 2-(3-chloro-4-fluorophenyl)-2H-1,2,3-triazole-4-carboxylic acid (141 mg, 584 μmol) in DMF (12 ml) was added 1-ethyl-3-(3-dimethylaminopropyl)carbodiimide hydrochloride (146 mg, 759 μmol), 1-hydroxybenzotriazole hydrate (116 mg, 759 μmol) and N,N-diisopropylethylamine (280 μl, 1.6 mmol). The reaction mixture was stirred overnight, then treated with water and extracted with ethyl acetate. The combined organic phases were concentrated under reduced pressure. The crude product was first purified by preparative HPLC (Column: Chromatorex C18 10 μm; 125×30 mm, Eluent A: water+0.05% trifluoroacetic acid, Eluent B: acetonitrile+0.05% trifluoroacetic acid; Gradient: 10% B→90% B) A second purification by preparative chiral HPLC [sample preparation: 7.8 mg dissolved in a mixture of ethanol and DCM; injection volume: 1 ml; column: Daicel Chiralpack IC 5 μm 250*20 mm; eluent: 75% n-heptane/25% ethanol; flow rate: 20 ml/min; temperature: 40° C.; UV detection: 220 nm] gave 3.10 mg (99% purity, 1% yield) of the desired product

Analytical chiral HPLC: Rₜ=7.12 min, e.e. =>99% [column: 250×4.6 mm filled with Daicel Chiralpak Ic 5 μm; eluent: 75% n-heptane/25% ethanol; flow rate: 1 ml/min; temperature: 40° C.; UV detection: 220 nm]

¹H-NMR (500 MHz, DMSO-d6) δ [ppm]: 0.844 (15.48), 0.858 (16.00), 0.956 (14.86), 0.970 (15.30), 1.087 (0.44), 1.234 (0.87), 1.973 (0.96), 1.986 (2.36), 1.999 (3.06), 2.013 (2.19), 2.026 (0.87), 2.086 (0.52), 2.363 (0.70), 2.638 (0.79), 3.278 (1.40), 3.292 (3.15), 3.367 (2.19), 3.380 (1.49), 3.424 (0.44), 3.436 (0.44), 3.564 (1.92), 3.577 (2.19), 3.592 (3.41), 3.605 (3.06), 3.667 (2.97), 3.679 (3.23), 3.694 (2.19), 3.706 (1.92), 7.688 (3.23), 7.706 (6.47), 7.724 (3.32), 7.808 (8.04), 8.051 (2.01), 8.058 (2.54), 8.065 (2.27), 8.069 (2.10), 8.075 (2.45), 8.083 (1.84), 8.246 (3.50), 8.252 (3.41), 8.259 (3.58), 8.264 (3.06), 8.438 (2.10), 8.451 (4.20), 8.464 (2.01), 8.504 (14.25), 10.661 (3.93).

### Example 127

### rac-2-(4-fluorophenyl)-N-{[4-(1-methyl-1H-pyrazol-5-yl)-2,5-dioxoimidazolidin-4-yl]methyl}-2H-1,2,3-triazole-4-carboxamide

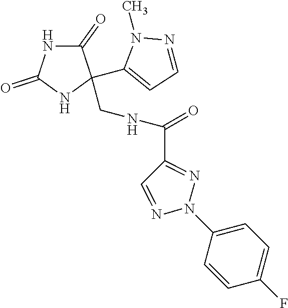 [SMILES: Cn1nccc1C1(CNC(=O)c2cnn(-c3ccc(F)cc3)n2)NC(=O)NC1=O]

To a solution of rac-5-(aminomethyl)-5-(1-methyl-1H-pyrazol-5-yl)imidazolidine-2,4-dione hydrochloride (100 mg, 78% purity, 317 μmol) and 2-(4-fluorophenyl)-2H-1,2,3-triazole-4-carboxylic acid (65.8 mg, 317 μmol) in DMF (1.6 ml) was added 1-ethyl-3-(3-dimethylaminopropyl)carbodiimide hydrochloride (79.1 mg, 413 μmol), 1-hydroxybenzotriazole hydrate (63.2 mg, 413 μmol) and N,N-diisopropylethylamine (280 μl, 1.6 mmol). The reaction mixture was stirred overnight at RT, after which extra portion of N,N-diisopropylethylamine (170 μl, 950 μmol) was added due to incomplete conversion. After stirring for 2 h at 50° C. and overnight at RT, the resulting reaction mixture was concentrated under reduced pressure and the crude product was purified by preparative HPLC (Method 3f). After lyophilization, 5.10 mg (99% purity, 4% yield) of the desired product were obtained.

LC-MS (Method 7): Rₜ=1.32 min; MS (ESIpos): m/z=399 [M+H]⁺

¹H-NMR (500 MHz, DMSO-d6) δ [ppm]: 0.006 (0.57), 2.073 (0.66), 3.808 (16.00), 4.017 (0.64), 4.029 (0.72), 4.044 (1.34), 4.057 (1.20), 4.086 (1.21), 4.099 (1.31), 4.113 (0.67), 4.127 (0.62), 6.555 (3.41), 6.559 (3.48), 7.401 (3.57), 7.405 (3.56), 7.468 (2.20), 7.473 (1.03), 7.486 (3.66), 7.499 (0.84), 7.503 (2.23), 8.083 (2.23), 8.087 (1.15), 8.093 (2.39), 8.097 (1.57), 8.101 (2.39), 8.106 (1.03), 8.111 (2.20), 8.436 (3.26), 8.489 (6.59), 8.705 (0.77), 8.718 (1.54), 8.731 (0.75), 11.211 (1.02).

### Example 128

### rac-N-({4-[1-(difluoromethyl)-1H-pyrazol-5-yl]-2,5-dioxoimidazolidin-4-yl}methyl)-2-(4-fluorophenyl)-2H-1,2,3-triazole-4-carboxamide

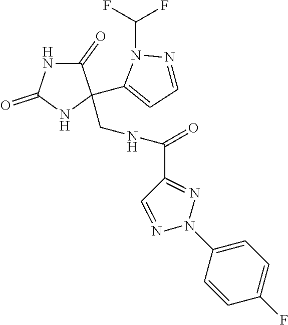 [SMILES: O=C1NC(=O)C(CNC(=O)c2cnn(-c3ccc(F)cc3)n2)(c2ccnn2C(F)F)N1]

To a solution of rac-5-(aminomethyl)-5-[1-(difluoromethyl)-1H-pyrazol-5-yl]imidazolidine-2,4-dione hydrochloride (26.5 mg, 94.1 μmol) and 2-(4-fluorophenyl)-2H-1,2,3-triazole-4-carboxylic acid (19.5 mg, 94.1 μmol) in DMF (460 μl) was added 1-ethyl-3-(3-dimethylaminopropyl)carbodiimide hydrochloride (23.4 mg, 122 μmol), 1-hydroxybenzotriazole hydrate (18.7 mg, 122 μmol) and N,N-diisopropylethylamine (82 μl, 470 μmol). After stirring overnight at RT, extra portion of N,N-diisopropylethylamine (49 μl, 280 μmol) was added and the reaction mixture was stirred for 2 h at 50° C. and concentrated under reduced pressure. The crude product was purified by preparative HPLC (Method 3f). After lyophilization, 16.9 mg (99% purity, 41% yield) of the desired product were obtained

LC-MS (Method 7): Rₜ=1.45 min; MS (ESIpos): m/z=435 [M+H]⁺

¹H-NMR (500 MHz, DMSO-d6) δ [ppm]: −0.006 (1.41), 0.007 (0.44), 2.073 (0.82), 2.362 (0.53), 3.990 (0.87), 4.003 (0.97), 4.017 (3.35), 4.032 (5.28), 4.045 (3.25), 4.060 (0.92), 4.073 (0.78), 6.770 (7.03), 6.773 (6.79), 7.464 (0.58), 7.471 (4.80), 7.475 (1.89), 7.488 (8.00), 7.501 (1.84), 7.506 (4.99), 7.513 (0.58), 7.802 (6.74), 7.805 (6.50), 7.859 (1.84), 7.970 (1.84), 7.978 (1.75), 8.075 (0.78), 8.082 (5.19), 8.086 (2.86), 8.091 (6.59), 8.096 (3.49), 8.100 (5.38), 8.105 (2.13), 8.109 (4.85), 8.116 (0.58), 8.492 (16.00), 8.505 (0.44), 8.595 (7.27), 8.828 (1.70), 8.840 (3.49), 8.853 (1.70), 11.216 (2.18).

### Example 129

### ent-N-({4-[1-(difluoromethyl)-1H-pyrazol-5-yl]-2,5-dioxoimidazolidin-4-yl}methyl)-2-(4-fluorophenyl)-2H-1,2,3-triazole-4-carboxamide

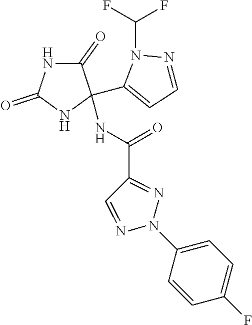 [SMILES: O=C1NC(=O)C(NC(=O)c2cnn(-c3ccc(F)cc3)n2)(c2ccnn2C(F)F)N1]

Enantiomeric separation of rac-N-({4-[1-(difluoromethyl)-1H-pyrazol-5-yl]-2,5-dioxoimidazolidin-4-yl}methyl)-2-(4-fluorophenyl)-2H-1,2,3-triazole-4-carboxamide (15.1 mg) by preparative chiral HPLC [column: Daicel Chiralpak ID 5 μm, 250×20 mm; eluent: 50% n-heptan/50% ethanol; flow rate: 15 ml/min; temperature: 30° C.; UV detection: 220 nm] afforded 4.8 mg of the desired product (97% purity).

Analytical chiral HPLC: Rₜ=6.93 min, e.e. =99% [column: 250×4.6 mm filled with Daicel Chiralpak ID 5 μm; eluent: 50% n-heptan/50% ethanol; flow rate: 1 ml/min; temperature: 40° C.; UV detection: 220 nm]

¹H-NMR (400 MHz, DMSO-d6) δ [ppm]: −0.149 (0.62), −0.008 (4.76), 0.008 (5.39), 0.146 (0.55), 0.844 (0.43), 0.858 (0.59), 0.874 (0.43), 1.128 (1.09), 1.146 (2.15), 1.164 (1.09), 1.235 (1.17), 2.073 (0.66), 2.323 (0.94), 2.327 (1.29), 2.332 (0.94), 2.366 (1.25), 2.523 (5.89), 2.665 (10.05), 2.670 (1.37), 2.674 (1.01), 2.710 (1.25), 2.893 (0.70), 2.911 (0.66), 3.980 (0.70), 3.997 (0.82), 4.015 (4.18), 4.027 (4.80), 4.032 (4.88), 4.042 (4.29), 4.061 (0.86), 4.077 (0.70), 6.767 (8.16), 6.771 (8.27), 7.466 (5.81), 7.472 (2.07), 7.489 (9.64), 7.505 (2.11), 7.510 (6.36), 7.519 (0.70), 7.800 (7.06), 7.804 (7.06), 7.829 (2.42), 7.969 (2.34), 7.978 (2.22), 8.069 (0.66), 8.078 (5.97), 8.083 (2.46), 8.090 (6.32), 8.095 (3.75), 8.101 (6.28), 8.107 (2.42), 8.113 (6.24), 8.492 (16.00), 8.586 (3.98), 8.822 (1.87), 8.838 (4.06), 8.854 (1.91).

### Example 130

### rac-N-{[4-(1-ethyl-1H-pyrazol-5-yl)-2,5-dioxoimidazolidin-4-yl]methyl}-2-(4-fluorophenyl)-2H-1,2,3-triazole-4-carboxamide

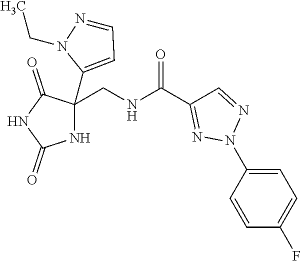 [SMILES: CCn1nccc1C1(CNC(=O)c2cnn(-c3ccc(F)cc3)n2)NC(=O)NC1=O]

2-(4-fluorophenyl)-2H-1,2,3-triazole-4-carboxylic acid (21.5 mg, 104 μmol) dissolved in 3 ml dichloromethan was treated with N,N-diisopropylethylamine (51 μl, 290 μmol), 1-ethyl-3-(3-dimethylaminopropyl)carbodiimide hydrochloride (25.9 mg, 135 μmol) and 1-hydroxybenzotriazole hydrate (20.7 mg, 135 μmol) and stirred for 5 min at room temperature before rac-5-(aminomethyl)-5-(1-ethyl-1H-pyrazol-5-yl)imidazolidine-2,4-dione-hydrogen chloride (30 mg, 104 μmol, 90% purity) was added. The mixture was stirred at room temperature for 3 h. Purification was done by preparative HPLC (Instrument: Waters Prep LC/MS System, column: Phenomenex Kinetex C18 5 μm 100×30 mm; eluent A: water, eluent B: acetonitrile, eluent C: 2% formic acid in water, eluent D: acetonitrile/water (80 Vol. %/20 Vol %) flow: 80 ml/min; room temperature, UV 200-400 nm, At-Column Injektion. Gradient: eluent A 0 to 2 min 63 ml, eluent B 0 to 2 min 7 ml, eluent A 2 to 10 min from 63 ml to 39 ml and eluent B from 7 ml to 31 ml, 10 to 12 min 0 ml eluent A and 70 ml eluent B. Eluent C and eluent D constant flow each 5 ml/min over the whole time). Further purification was needed by preparative HPLC (Column: 250*20 mm Daicel Chiralpak AZ-H 5 μm; eluent: 50% n-heptane, 50%2-propanol; flow: 15 ml/min; temperature: 40° C.). Product containing samples were united, the solvents were evaporated and the residue was lyophylized. 5.00 mg (100% purity, 12% yield) of the title compound were obtained.

LC-MS (Method 7): Rₜ=1.32 min; MS (ESIpos): m/z=413 [M+H]⁺

¹H-NMR (500 MHz, DMSO-d6) δ [ppm]: 1.300 (7.74), 1.314 (16.00), 1.329 (7.62), 2.073 (4.46), 4.006 (0.87), 4.021 (2.29), 4.035 (4.93), 4.049 (8.24), 4.064 (6.82), 4.070 (3.73), 4.078 (2.76), 4.084 (3.35), 4.092 (1.30), 4.098 (1.25), 4.111 (1.06), 6.548 (6.89), 6.552 (6.63), 7.460 (7.69), 7.464 (7.67), 7.469 (5.03), 7.487 (7.76), 7.504 (4.46), 8.085 (4.74), 8.095 (5.07), 8.099 (3.49), 8.103 (4.86), 8.113 (4.34), 8.461 (7.01), 8.492 (12.53), 8.700 (1.72), 8.713 (3.40), 8.726 (1.68), 11.213 (3.19).

### Example 131

### rac-2-(4-fluorophenyl)-N-{[4-(4-methyl-1,3-thiazol-5-yl)-2,5-dioxoimidazolidin-4-yl]methyl}-2H-1,2,3-triazole-4-carboxamide

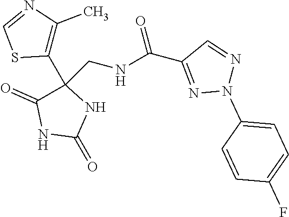 [SMILES: Cc1ncsc1C1(CNC(=O)c2cnn(-c3ccc(F)cc3)n2)NC(=O)NC1=O]

2-(4-fluorophenyl)-2H-1,2,3-triazole-4-carboxylic acid (78.9 mg, 381 μmol) dissolved in 10 ml dichloromethane was treated with N,N-diisopropylethylamine (190 μl, 1.1 mmol), 1-ethyl-3-(3-dimethylaminopropyl)carbodiimide hydrochloride (94.9 mg, 495 μmol) and 1-hydroxybenzotriazole hydrate (75.8 mg, 495 μmol) and stirred for 5 min at room temperature before rac-5-(aminomethyl)-5-(4-methyl-1,3-thiazol-5-yl)imidazolidine-2,4-dione-hydrogen chloride (100 mg, 381 μmol) was added. The mixture was stirred at room temperature over night. Acetonitrile was added, the precipitate was filtered of, washed with acetonitrile and dried in vacuo. 40.0 mg (100% purity, 25% yield) of the title compound were obtained.

LC-MS (Method 7): Rₜ=1.25 min; MS (ESIpos): m/z=416 [M+H]⁺

¹H-NMR (400 MHz, DMSO-d6) δ [ppm]: 2.501 (16.00), 4.034 (1.44), 4.041 (1.48), 4.049 (1.44), 4.057 (1.39), 7.465 (1.52), 7.470 (0.56), 7.487 (2.76), 7.504 (0.60), 7.509 (1.65), 8.073 (1.59), 8.085 (1.68), 8.090 (1.03), 8.096 (1.64), 8.107 (1.52), 8.476 (3.81), 8.597 (2.44), 8.682 (0.59), 8.698 (1.24), 8.714 (0.59), 8.927 (2.64), 11.171 (1.28).

### Example 132

### N-{[(4R)-4-cyclopropyl-2,5-dioxoimidazolidin-4-yl]methyl}-2-(1,2-thiazol-4-yl)-2H-1,2,3-triazole-4-carboxamide

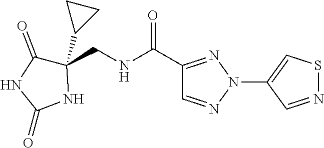 [SMILES: O=C1NC(=O)[C@](CNC(=O)c2cnn(-c3cnsc3)n2)(C2CC2)N1]

2-(1,2-thiazol-4-yl)-2H-1,2,3-triazole-4-carboxylic acid (45.0 mg, 229 μmol) dissolved in 5 ml dichloromethane was treated with N,N-diisopropylethylamine (110 μl, 640 μmol), 1-ethyl-3-(3-dimethylaminopropyl)carbodiimide hydrochloride (57.2 mg, 298 μmol) and 1-hydroxybenzotriazole hydrate (45.7 mg, 298 μmol) and stirred for 5 min at room temperature before (5R)-5-(aminomethyl)-5-cyclopropylimidazolidine-2,4-dione hydrochloride (47.2 mg, 229 μmol) was added. The mixture was stirred at room temperature over night. Purification was done by preparative HPLC (column: Chromatorex C18 10 μm, 250×30 mm; eluent A=water, B=acetonitrile; gradient: 0.0 min 5% B; 3 min 5% B; 20 min 50% B; 23 min 100% B; 26 min 5% B; flow: 50 ml/min; 0.1% formic acid). Product containing samples were united, the solvents were evaporated and the residue was lyophylized. 54.0 mg (100% purity, 68% yield) of the title compound were obtained.

LC-MS (Method 7): Rₜ=0.96 min; MS (ESIpos): m/z=348 [M+H]⁺

¹H-NMR (400 MHz, DMSO-d6) δ [ppm]: 0.105 (0.59), 0.116 (1.19), 0.128 (1.67), 0.137 (2.23), 0.143 (1.75), 0.151 (1.56), 0.166 (0.83), 0.308 (0.50), 0.328 (1.47), 0.337 (1.77), 0.346 (2.32), 0.355 (1.73), 0.365 (1.28), 0.376 (1.05), 0.417 (2.60), 0.435 (5.25), 0.452 (4.72), 0.472 (1.77), 1.133 (0.78), 1.148 (1.53), 1.153 (1.64), 1.167 (2.49), 1.182 (1.64), 1.201 (0.65), 2.367 (0.40), 3.633 (1.67), 3.648 (1.84), 3.667 (3.79), 3.682 (3.47), 3.714 (3.44), 3.731 (3.69), 3.748 (1.75), 3.765 (1.67), 7.616 (6.65), 8.487 (2.15), 8.495 (16.00), 8.502 (4.32), 8.518 (2.06), 9.100 (11.53), 9.459 (10.74), 10.650 (5.43).

### Example 133

### rac-N-({2,5-dioxo-4-[1-(2,2,2-trifluoroethyl)-1H-pyrazol-5-yl]imidazolidin-4-yl}methyl)-2-(4-fluorophenyl)-2H-1,2,3-triazole-4-carboxamide

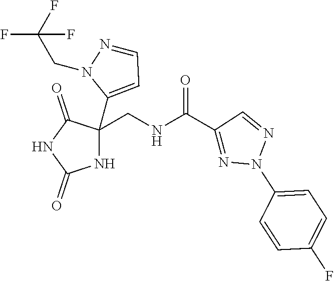 [SMILES: O=C1NC(=O)C(CNC(=O)c2cnn(-c3ccc(F)cc3)n2)(c2ccnn2CC(F)(F)F)N1]

2-(4-fluorophenyl)-2H-1,2,3-triazole-4-carboxylic acid (19.8 mg, 95.6 μmol) dissolved in 3 ml dichloromethane was treated with N,N-diisopropylethylamine (47 μl, 270 μmol), 1-ethyl-3-(3-dimethylaminopropyl)carbodiimide hydrochloride (23.8 mg, 124 μmol) and 1-hydroxybenzotriazole hydrate (19.0 mg, 124 μmol) and stirred for 5 min at room temperature before rac-5-(aminomethyl)-5-[1-(2,2,2-trifluoroethyl)-1H-pyrazol-5-yl]imidazolidine-2,4-dione hydrochloride (30.0 mg, 95.6 μmol) was added. The mixture was stirred at room temperature for 2 h. Purification was done by preparative HPLC (Instrument: Waters Prep LC/MS System, column: Phenomenex Kinetex C18 5 μm 100×30 mm; eluent A: water, eluent B: acetonitrile, eluent C: 2% formic acid in water, eluent D: acetonitrile/water (80 Vol. %/20 Vol %) flow: 80 ml/min; room temperature, UV 200-400 nm, At-Column Injektion. Gradient: eluent A 0 to 2 min 63 ml, eluent B 0 to 2 min 7 ml, eluent A 2 to 10 min from 63 ml to 39 ml and eluent B from 7 ml to 31 ml, 10 to 12 min 0 ml eluent A and 70 ml eluent B. Eluent C and eluent D constant flow each 5 ml/min over the whole time). Product containing samples were united, the solvents were evaporated and the residue was lyophylized. 18.0 mg (100% purity, 40% yield) of the title compound were obtained.

LC-MS (Method 7): Rₜ=1.53 min; MS (ESIpos): m/z=467 [M+H]⁺

¹H-NMR (600 MHz, DMSO-d6) δ [ppm]: 0.005 (0.62), 2.520 (1.50), 3.967 (1.50), 3.979 (1.68), 3.990 (2.78), 4.002 (2.55), 4.039 (2.51), 4.049 (2.87), 4.062 (1.73), 4.072 (1.52), 5.153 (0.54), 5.164 (0.91), 5.179 (2.69), 5.194 (3.71), 5.208 (2.54), 5.220 (1.07), 5.234 (0.58), 6.654 (7.65), 6.658 (7.86), 7.468 (0.47), 7.474 (4.62), 7.478 (1.76), 7.489 (7.44), 7.500 (1.83), 7.503 (4.94), 7.509 (0.73), 7.628 (8.16), 7.632 (8.06), 8.077 (0.49), 8.082 (4.62), 8.086 (2.04), 8.090 (4.97), 8.094 (3.09), 8.098 (5.10), 8.102 (2.09), 8.106 (4.78), 8.111 (0.70), 8.488 (16.00), 8.503 (7.46), 8.785 (1.64), 8.796 (3.34), 8.807 (1.72), 11.261 (2.81).

### Example 134

### N-{[(4R)-4-cyclopropyl-2,5-dioxoimidazolidin-4-yl]methyl}-2-(3,5-difluoropyridin-2-yl)-2H-1,2,3-triazole-4-carboxamide

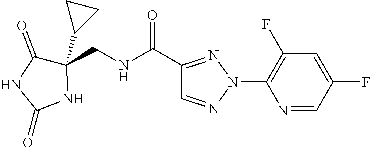 [SMILES: O=C1NC(=O)[C@](CNC(=O)c2cnn(-c3ncc(F)cc3F)n2)(C2CC2)N1]

2-(3,5-difluoropyridin-2-yl)-2H-1,2,3-triazole-4-carboxylic acid (8.00 mg, 35.4 μmol) dissolved in 1 ml dichloromethane was treated with N,N-diisopropylethylamine (17 μl, 99 μmol), 1-ethyl-3-(3-dimethylaminopropyl)carbodiimide hydrochloride (8.82 mg, 46.0 μmol) and 1-hydroxybenzotriazole hydrate (7.04 mg, 46.0 μmol) and stirred for 5 min at room temperature before (5R)-5-(aminomethyl)-5-cyclopropylimidazolidine-2,4-dione hydrochloride (7.27 mg, 35.4 μmol) was added. The mixture was stirred at room temperature over night. Purification was done by preparative HPLC (Instrument: Waters Prep LC/MS System, column: Phenomenex Kinetex C18 5 μm 100×30 mm; eluent A: water, eluent B: acetonitrile, eluent C: 2% formic acid in water, eluent D: acetonitrile/water (80 Vol. %/20 Vol %), flow: 80 ml/min; room temperature, UV 200-400 nm, At-Column Injektion; Gradient: eluent A 0 to 2 min 63 ml, eluent B 0 to 2 min 7 ml, eluent A 2 to 10 min from 63 ml to 39 ml and eluent B from 7 ml to 31 ml, 10 to 12 min 0 ml eluent A and 70 ml eluent B. Eluent C and eluent D constant flow each 5 ml/min over the whole time). Product containing samples were united, the solvents were evaporated and the residue was lyophylized. 5.00 mg (100% purity, 37% yield) of the title compound were obtained.

LC-MS (Method 7): Rₜ=0.98 min; MS (ESIpos): m/z=378 [M+H]⁺

¹H-NMR (600 MHz, DMSO-d6) δ [ppm]: 0.005 (0.62), 0.104 (0.54), 0.111 (1.04), 0.119 (1.21), 0.123 (1.65), 0.131 (1.35), 0.137 (1.21), 0.145 (0.65), 0.310 (0.49), 0.317 (0.62), 0.323 (1.52), 0.332 (1.49), 0.335 (1.55), 0.342 (1.35), 0.346 (1.05), 0.356 (0.78), 0.409 (2.62), 0.421 (4.81), 0.433 (4.07), 0.444 (1.34), 1.135 (0.71), 1.147 (1.50), 1.157 (2.07), 1.168 (1.30), 1.180 (0.58), 2.074 (3.28), 2.424 (0.47), 2.520 (0.57), 2.522 (0.57), 2.654 (0.41), 3.695 (7.50), 3.705 (7.48), 7.603 (5.95), 8.426 (1.66), 8.430 (1.78), 8.440 (1.86), 8.444 (2.72), 8.447 (1.92), 8.457 (1.67), 8.461 (1.69), 8.532 (1.68), 8.543 (3.54), 8.553 (1.68), 8.574 (16.00), 8.635 (6.47), 8.639 (6.10), 10.653 (4.29).

### Example 135

### rac-N-({2,5-dioxo-4-[5-(trifluoromethyl)-1,3-thiazol-4-yl]imidazolidin-4-yl}methyl)-2-(4-fluorophenyl)-2H-1,2,3-triazole-4-carboxamide

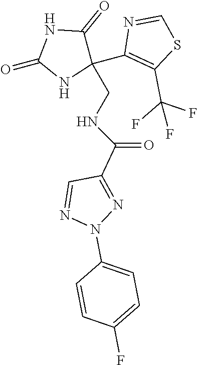 [SMILES: O=C1NC(=O)C(CNC(=O)c2cnn(-c3ccc(F)cc3)n2)(c2ncsc2C(F)(F)F)N1]

To a solution of Rac-5-(aminomethyl)-5-[5-(trifluoromethyl)-1,3-thiazol-4-yl]imidazolidine-2,4-dione hydrochloride (100 mg, 316 μmol) and 2-(4-fluorophenyl)-2H-1,2,3-triazole-4-carboxylic acid (78.5 mg, 379 μmol) in DMF (2.0 ml) was added 1-ethyl-3-(3-dimethylaminopropyl)carbodiimide hydrochloride (78.7 mg, 410 μmol), 1-hydroxybenzotriazole hydrate (62.9 mg, 410 μmol) and N,N-diisopropylethylamine (280 μl, 1.6 mmol). The reaction mixture was stirred for 2 days at RT and concentrated under reduced pressure. The crude product was purified by preparative HPLC (Method 4f). After lyophilisation, 81.0 mg (100% purity, 55% yield) of the desired product were obtained.

LC-MS (Method 7): Rₜ=1.46 min; MS (ESIpos): m/z=470 [M+H]⁺

¹H-NMR (600 MHz, DMSO-d6) δ [ppm]: 2.074 (0.92), 2.392 (1.19), 2.426 (0.78), 2.523 (0.87), 2.655 (0.78), 2.891 (0.41), 3.289 (0.64), 3.298 (1.65), 3.303 (1.70), 3.363 (0.78), 3.369 (1.05), 4.166 (2.61), 4.177 (2.84), 4.189 (3.35), 4.200 (3.03), 4.397 (3.16), 4.408 (3.44), 4.420 (2.84), 4.431 (2.61), 7.468 (5.59), 7.483 (10.27), 7.498 (5.78), 8.082 (5.87), 8.090 (6.10), 8.093 (3.81), 8.097 (6.01), 8.105 (5.64), 8.336 (8.80), 8.485 (16.00), 8.497 (2.38), 8.507 (4.77), 8.518 (2.25), 9.451 (13.57), 11.087 (5.55).

### Example 136

### ent-N-({2, 5-dioxo-4-[5-(trifluoromethyl)-1,3-thiazol-4-yl]imidazolidin-4-yl}methyl)-2-(4-fluorophenyl)-2H-1,2,3-triazole-4-carboxamide

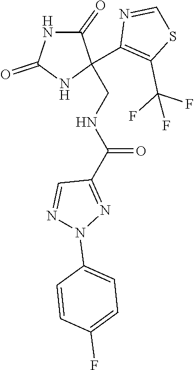 [SMILES: O=C1NC(=O)C(CNC(=O)c2cnn(-c3ccc(F)cc3)n2)(c2ncsc2C(F)(F)F)N1]

Enantiomeric separation of rac-N-({2,5-dioxo-4-[5-(trifluoromethyl)-1,3-thiazol-4-yl]imidazolidin-4-yl}methyl)-2-(4-fluorophenyl)-2H-1,2,3-triazole-4-carboxamide (80 mg) by preparative chiral HPLC [column: Daicel Chiralcel OD-H 5 μm, 250×20 mm; eluent: 70% n-heptane/30% ethanol+0.2% diethylamine; flow rate: 20 ml/min; temperature: 40° C.; UV detection: 220 nm] afforded 27.86 mg of the desired product (96% purity).

Analytical chiral HPLC: Rₜ=0.92 min, e.e. =99% [column: 50×4.6 mm filled with Phenomenex cellulose-1 3 μm; eluent: 50% n-heptan/50% ethanol+0.2% diethylamine; flow rate: 1 ml/min; UV detection: 220 nm]

LC-MS (Method 7): Rₜ=1.46 min; MS (ESIpos): m/z=470 [M+H]⁺

¹H-NMR (400 MHz, DMSO-d₆) δ[ppm]: −0.008 (0.91), 0.008 (1.07), 0.845 (0.42), 0.858 (0.52), 1.053 (2.18), 1.071 (4.44), 1.089 (2.38), 1.103 (0.54), 1.236 (0.50), 2.328 (0.52), 2.366 (0.67), 2.523 (2.16), 2.666 (0.50), 2.670 (0.67), 2.675 (0.57), 2.710 (1.69), 2.724 (1.13), 4.157 (1.92), 4.172 (2.14), 4.191 (2.68), 4.207 (2.42), 4.385 (2.44), 4.402 (2.72), 4.419 (2.14), 4.436 (1.90), 7.451 (0.46), 7.460 (4.98), 7.466 (1.71), 7.482 (8.13), 7.491 (0.95), 7.498 (1.78), 7.504 (5.47), 7.512 (0.57), 8.066 (0.57), 8.075 (5.12), 8.081 (2.04), 8.087 (5.39), 8.092 (3.15), 8.098 (5.37), 8.104 (1.88), 8.110 (4.96), 8.119 (0.48), 8.321 (1.92), 8.483 (16.00), 8.497 (3.55), 8.513 (1.61), 9.449 (8.68).

### Example 137

### ent-N-[(4-sec-butyl-2,5-dioxoimidazolidin-4-yl)methyl]-2-(4-fluorophenyl)-2H-1,2,3-triazole-4-carboxamide

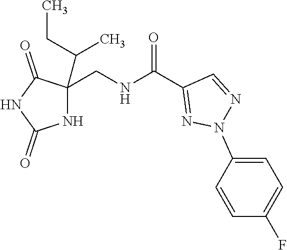 [SMILES: CCC(C)C1(CNC(=O)c2cnn(-c3ccc(F)cc3)n2)NC(=O)NC1=O]

Enantiomeric separation of diamix-N-[(4-sec-butyl-2,5-dioxoimidazolidin-4-yl)methyl]-2-(4-fluorophenyl)-2H-1,2,3-triazole-4-carboxamide (120 mg; 321 μmol) using the following method
Column: Daicel Chiralpak OZ-H 5 μm 250×20 mm
Solvent: 70% n-heptane/30% ethanol
Flow: 15 ml/min
Column Temperature: 40° C.
   afforded 27 mg (100% purity, 23% yield) of the desired product.

LC-MS (Method 7): Rₜ=1.56 min; MS (ESIpos): m/z=375 [M+H]⁺

Chiral HPLC (Column: Daicel Chiralpak OZ-H 5 μm 250×4.6 mm; eluent: 70% n-heptane/30% ethanole; flow: 1 ml/min; column temperature: 35° C.): Rₜ=6.538 min, >99.0% ee

¹H-NMR (400 MHz, DMSO-d6) δ [ppm]: −0.008 (2.73), 0.008 (2.62), 0.852 (13.18), 0.869 (16.00), 0.884 (13.14), 0.902 (7.15), 0.987 (0.75), 1.004 (0.88), 1.014 (1.17), 1.019 (1.30), 1.031 (1.20), 1.046 (1.13), 1.064 (0.68), 1.618 (1.08), 1.637 (1.28), 1.651 (1.53), 1.669 (2.25), 1.685 (1.67), 1.694 (1.67), 1.701 (1.22), 1.711 (1.15), 1.718 (0.86), 3.611 (1.15), 3.627 (1.28), 3.645 (3.27), 3.661 (3.08), 3.676 (3.06), 3.691 (3.21), 3.710 (1.22), 3.725 (1.12), 7.450 (0.46), 7.459 (4.71), 7.464 (1.69), 7.481 (8.61), 7.497 (1.73), 7.503 (5.18), 7.511 (0.57), 7.754 (7.70), 8.064 (0.53), 8.072 (4.96), 8.078 (2.07), 8.084 (5.25), 8.090 (3.09), 8.095 (5.16), 8.107 (4.82), 8.116 (0.50), 8.323 (1.67), 8.339 (3.50), 8.354 (1.66), 8.465 (12.09), 10.655 (3.88).

### Example 138

### ent-N-{[2,5-dioxo-4-(pyridin-2-yl)imidazolidin-4-yl]methyl}-2-(4-fluorophenyl)-2H-1,2,3-triazole-4-carboxamide

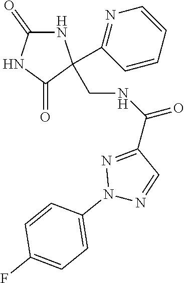 [SMILES: O=C1NC(=O)C(CNC(=O)c2cnn(-c3ccc(F)cc3)n2)(c2ccccn2)N1]

Enantiomeric separation of rac-N-{[2,5-dioxo-4-(pyridin-2-yl)imidazolidin-4-yl]methyl}-2-(4-fluorophenyl)-2H-1,2,3-triazole-4-carboxamide (31.7 mg) by preparative chiral HPLC [column: Daicel Chiralcel® OX-H 5 μm, 250×30 mm; eluent: 50% n-heptane/50% ethanol+0.2% diethylamine; flow rate: 40 ml/min; temperature: 40° C.; UV detection: 220 nm] afforded 14 mg of the desired product. The product was further purified by preparative HPLC (Method 1f) The combined organic phases were concentrated in vacuo and the remaining TFA was removed, using a Stratospheres PL-HCO3 MP SPE cartridge. After lyophilization, 4.1 mg of the desired product were obtained.

Analytical chiral HPLC: Rₜ=9.61 min, e.e. =99% [column: Daicel Chiralcel® OX-H 5 μm, 250×30 mm; eluent: 50% n-heptane/50% ethanol+0.2% diethylamine; flow rate: 40 ml/min; temperature: 40° C.; UV detection: 220 nm]

¹H-NMR (500 MHz, DMSO-d6) δ [ppm]: 2.095 (0.66), 2.364 (0.47), 2.637 (0.47), 3.091 (0.47), 4.082 (1.93), 4.095 (2.12), 4.110 (2.99), 4.122 (2.63), 4.214 (2.58), 4.226 (2.92), 4.241 (2.06), 4.254 (1.80), 7.398 (2.56), 7.408 (2.80), 7.412 (2.78), 7.423 (2.69), 7.461 (5.11), 7.465 (1.97), 7.479 (8.84), 7.496 (5.43), 7.545 (4.75), 7.561 (5.17), 7.859 (2.25), 7.862 (2.27), 7.874 (3.71), 7.878 (3.71), 7.889 (1.95), 7.893 (1.95), 8.072 (5.24), 8.082 (5.57), 8.086 (3.47), 8.090 (5.59), 8.100 (5.21), 8.364 (2.22), 8.475 (16.00), 8.502 (1.80), 8.515 (3.64), 8.527 (1.70), 8.633 (3.58), 8.641 (3.50), 10.912 (0.97).

### Example 139

### rac-N-[(2,5-dioxo-4-phenylimidazolidin-4-yl)methyl]-2-(4-fluorophenyl)-2H-1,2,3-triazole-4-carboxamide

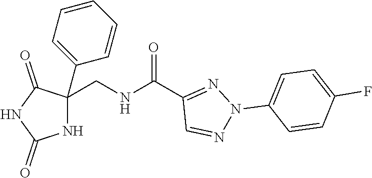 [SMILES: O=C1NC(=O)C(CNC(=O)c2cnn(-c3ccc(F)cc3)n2)(c2ccccc2)N1]

2-(4-fluorophenyl)-2H-1,2,3-triazole-4-carboxylic acid (42.9 mg, 207 μmol) dissolved in 2 ml DMF was treated with N,N-diisopropylethylamine (100 μl, 580 μmol), 1-ethyl-3-(3-dimethylaminopropyl)carbodiimide hydrochloride (51.6 mg, 269 μmol) and 1-hydroxybenzotriazole hydrate (41.2 mg, 269 μmol) and stirred for 5 min at room temperature before rac-5-(aminomethyl)-5-phenylimidazolidine-2,4-dione hydrochloride (50.0 mg, 207 μmol) was added. The mixture was stirred at room temperature for 2 h. Purification was done by preparative HPLC (Instrument: Waters Prep LC/MS System, column: Phenomenex Kinetex 018 5 μm 100×30 mm; eluent A: water, eluent B: acetonitrile, eluent C: 2% formic acid in water, eluent D: acetonitrile/water (80 Vol. %/20 Vol %) flow: 80 ml/min; room temperature, UV 200-400 nm, At-Column Injektion. Gradient: eluent A 0 to 2 min 63 ml, eluent B 0 to 2 min 7 ml, eluent A 2 to 10 min from 63 ml to 39 ml and eluent B from 7 ml to 31 ml, 10 to 12 min 0 ml eluent A and 70 ml eluent B. Eluent C and eluent D constant flow each 5 ml/min over the whole time). Product containing samples were united, the solvents were evaporated and the residue was lyophylized. 25.0 mg (100% purity, 31% yield) of the title compound were obtained.

LC-MS (Method 7): Rₜ=1.49 min; MS (ESIpos): m/z=395 [M+H]⁺

¹H-NMR (400 MHz, DMSO-d6) δ [ppm]: −0.008 (1.46), 0.008 (1.53), 2.327 (0.97), 2.366 (1.11), 2.524 (3.61), 2.670 (0.90), 2.710 (1.11), 3.956 (6.39), 3.972 (6.39), 7.335 (1.18), 7.353 (4.06), 7.371 (3.33), 7.400 (4.96), 7.419 (7.57), 7.437 (3.30), 7.456 (4.89), 7.462 (1.67), 7.478 (7.81), 7.495 (1.70), 7.500 (5.31), 7.589 (7.08), 7.607 (6.59), 7.610 (4.30), 8.061 (5.03), 8.073 (5.31), 8.079 (3.05), 8.084 (5.24), 8.096 (4.82), 8.454 (16.00), 8.545 (1.67), 8.560 (8.68), 8.577 (1.60), 10.865 (4.34).

### Example 140

### rac-N-{[4-(1-methyl-1H-imidazol-2-yl)-2,5-dioxoimidazolidin-4-yl]methyl}-2-phenyl-2H-1,2,3-triazole-4-carboxamide

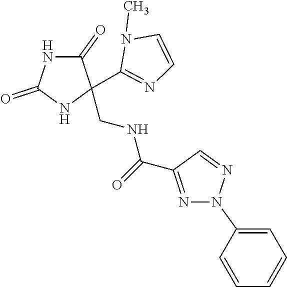 [SMILES: Cn1ccnc1C1(CNC(=O)c2cnn(-c3ccccc3)n2)NC(=O)NC1=O]

The compound was synthesized according to the General Method A using 193 mg (1.02 mmol) of 2-phenyl-2H-1,2,3-triazole-4-carboxylic acid, 250 mg (1.02 mmol) of rac-5-(aminomethyl)-5-(1-methyl-1H-imidazol-2-yl)imidazolidine-2,4-dione-hydrogen chloride, 293 mg (1.53 mmol) of 1-(3-dimethylaminopropyl)-3-ethylcarbodiimide hydrochloride, 1.50 mL (1.50 mmol) of 1M 1-hydroxy-7-azabenzotriazole in N,N-dimethylacetamide and 0.43 mL (3.10 mmol) of triethylamine in 2 mL of N,N-dimethylformamide. The crude product obtained from the workup was purified by flash column chromatography eluting with dichloromethane/methanol mixtures to give 8 mg (2% yield, 98% purity) of the compound form as a white powder.

¹H-NMR (300 MHz, DMSO-d₆): δ [ppm]=3.50 (s, 3H), 4.20 (d, 2H), 6.95 (s, 1H), 7.26 (s, 1H), 7.35 (t, 1H), 7.62 (t, 2H), 8.05 (d, 2H), 8.47 (d, 2H), 8.60 (t, 1H), 11.24 (bp, 1H).

LC-MS (Method 1): Rₜ=1.827 min. MS (Mass method 1): m/z=381 (M+H)⁺

### Example 141

### rac-2-(4-fluorophenyl)-N-{[4-(1-methyl-1H-imidazol-2-yl)-2,5-dioxoimidazolidin-4-yl]methyl}-2H-1,2,3-triazole-4-carboxamide

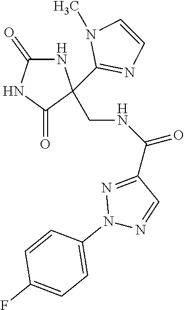 [SMILES: Cn1ccnc1C1(CNC(=O)c2cnn(-c3ccc(F)cc3)n2)NC(=O)NC1=O]

The compound was synthesized according to the General Method A using 295 mg (1.42 mmol) of 2-(4-fluorophenyl)-2H-1,2,3-triazole-4-carboxylic acid, 350 mg (1.42 mmol) of rac-5-(aminomethyl)-5-(1-methyl-1H-imidazol-2-yl)imidazolidine-2,4-dione-hydrogen chloride, 410 mg (2.14 mmol) of 1-(3-dimethylaminopropyl)-3-ethylcarbodiimide hydrochloride, 2.14 mL (2.14 mmol) of 1M 1-hydroxy-7-azabenzotriazole in N,N-dimethylacetamide and 0.60 mL (4.30 mmol) of triethylamine in 2 mL of N,N-dimethylformamide. The crude product obtained from the workup was purified by flash column chromatography eluting with dichloromethane/methanol mixtures to give 11 mg (2% yield, 97% purity) of the compound form as a white powder.

¹H-NMR (300 MHz, DMSO-d₆): δ [ppm]=3.50 (s, 3H), 4.19 (d, 2H), 6.94 (s, 1H), 7.25 (s, 1H), 7.48 (t, 2H), 8.04-8.12 (m, 2H), 8.44 (s, 1H), 8.48 (s, 1H), 8.59 (t, 1H), 11.23 (s, 1H).

LC-MS (Method 1): Rₜ=1.939 min. MS (Mass method 1): m/z=399 (M+H)⁺

### Example 142

### ent-N-{[4-(1-methyl-1H-imidazol-2-yl)-2,5-dioxoimidazolidin-4-yl]methyl}-2-phenyl-2H-1,2,3-triazole-4-carboxamide

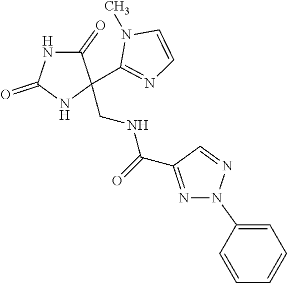 [SMILES: Cn1ccnc1C1(CNC(=O)c2cnn(-c3ccccc3)n2)NC(=O)NC1=O]

Enantiomeric separation of rac-N-{[4-(1-methyl-1H-imidazol-2-yl)-2,5-dioxoimidazolidin-4-yl]methyl}-2-phenyl-2H-1,2,3-triazole-4-carboxamide (6.6 mg) by preparative chiral HPLC [column: Daicel Chiralpak IF 5 μm, 250×20 mm; eluent: 60% n-heptane/40% isopropanol; flow rate: 20 ml/min; temperature: 45° C.; UV detection: 220 nm] afforded 1.44 mg of the desired product.

Analytical chiral HPLC: Rₜ=16.06 min, e.e. =>99% [column: 250×4.6 mm filled with Daicel Chiralpak IF 5 μm; eluent: 60% n-heptane/40% isopropanol; flow rate: 1 ml/min; temperature: 45° C.; UV detection: 220 nm]

¹H-NMR (500 MHz, DMSO-d6) δ [ppm]: 0.006 (0.75), 1.174 (0.45), 1.235 (0.80), 2.695 (0.87), 3.303 (0.50), 3.506 (16.00), 4.188 (3.52), 4.201 (3.45), 6.956 (4.18), 6.958 (4.27), 7.260 (3.88), 7.262 (3.94), 7.494 (0.85), 7.509 (2.04), 7.524 (1.33), 7.614 (2.49), 7.631 (3.55), 7.646 (2.02), 8.048 (3.58), 8.063 (3.47), 8.471 (3.71), 8.491 (7.49), 8.598 (0.83), 8.611 (1.76), 8.624 (0.81).

### Example 143

### rac-N-{[4-(1-methyl-1H-imidazol-2-yl)-2,5-dioxoimidazolidin-4-yl]methyl}-2-(3,4,5-trifluorophenyl)-2H-1,2,3-triazole-4-carboxamide

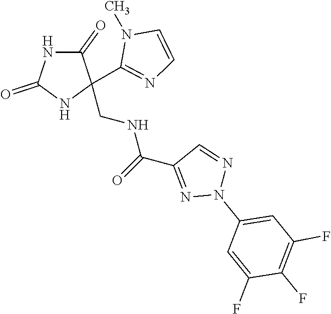 [SMILES: Cn1ccnc1C1(CNC(=O)c2cnn(-c3cc(F)c(F)c(F)c3)n2)NC(=O)NC1=O]

To a solution of rac-5-(aminomethyl)-5-(1-methyl-1H-imidazol-2-yl)imidazolidine-2,4-dione hydrochloride (80.0 mg) and 2-(3,4,5-trifluorophenyl)-2H-1,2,3-triazole-4-carboxylic acid (79.2 mg, 326 μmol) in dichloromethane (9 ml) was added 1-ethyl-3-(3-dimethylaminopropyl)carbodiimide hydrochloride (81.2 mg, 423 μmol), 1-hydroxybenzotriazole hydrate (64.8 mg, 423 μmol) and N,N-diisopropylethylamine (160 μl, 910 μmol). The reaction mixture was stirred overnight at RT and concentrated under reduced pressure. The crude product was purified by preparative HPLC (method 3f). After lyophilization, 10.5 mg (7.4% yield) of the desired product were obtained.

LC-MS (Method 8): Rₜ=0.69 min; MS (ESIpos): m/z=435 [M+H]⁺

¹H-NMR (500 MHz, DMSO-d₆) δ[ppm]: −0.120 (0.55), −0.007 (5.27), 0.006 (3.72), 0.117 (0.52), 3.505 (16.00), 4.141 (0.78), 4.155 (0.87), 4.169 (1.40), 4.182 (1.22), 4.228 (1.27), 4.241 (1.40), 4.256 (0.87), 4.269 (0.78), 6.938 (4.09), 6.941 (4.15), 7.255 (3.81), 7.257 (3.83), 8.006 (1.50), 8.018 (1.80), 8.022 (1.85), 8.035 (1.64), 8.444 (3.61), 8.572 (7.45), 8.684 (0.78), 8.697 (1.65), 8.710 (0.80), 11.241 (0.86).

### Example 144

### ent-2-(4-fluorophenyl)-N-{[4-(1-methyl-1H-imidazol-2-yl)-2,5-dioxoimidazolidin-4-yl]methyl}-2H-1,2,3-triazole-4-carboxamide

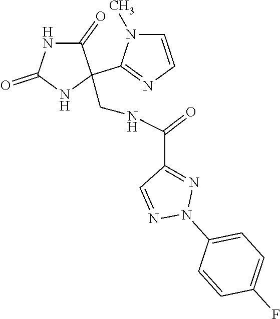 [SMILES: Cn1ccnc1C1(CNC(=O)c2cnn(-c3ccc(F)cc3)n2)NC(=O)NC1=O]

To a solution of ent-5-(aminomethyl)-5-(1-methyl-1H-imidazol-2-yl)imidazolidine-2,4-dione hydrochloride (1:1) (35.0 mg, 142 μmol) and 2-(4-fluorophenyl)-2H-1,2,3-triazole-4-carboxylic acid (29.5 mg, 142 μmol) in DMF (890 μl) was added 1-ethyl-3-(3-dimethylaminopropyl)carbodiimide hydrochloride (35.5 mg, 185 μmol), 1-hydroxybenzotriazole hydrate (28.4 mg, 185 μmol) and N,N-diisopropylethylamine (120 μl, 710 μmol). The reaction mixture was stirred overnight at RT and concentrated under reduced pressure. The crude product was purified by preparative HPLC (Methode 5f). After lyophilisation, 24.3 mg (42% yield, 98% purity) of the desired product were obtained.

LC-MS (Method 7): Rₜ=1.08 min; MS (ESIpos): m/z=399 [M+H]⁺

¹H-NMR (500 MHz, DMSO-d6) δ [ppm]: 3.505 (16.00), 4.186 (3.47), 4.199 (3.59), 6.948 (4.22), 6.950 (4.20), 7.257 (4.15), 7.468 (2.28), 7.472 (0.86), 7.486 (4.05), 7.503 (2.35), 8.068 (2.43), 8.073 (1.19), 8.078 (2.56), 8.082 (1.63), 8.086 (2.48), 8.096 (2.25), 8.460 (3.90), 8.490 (6.76), 8.599 (0.91), 8.612 (1.85), 8.625 (0.86), 11.246 (0.48).

### Example 145

### ent-2-(4-chloro-3-fluorophenyl)-N-{[4-(1-methyl-1H-imidazol-2-yl)-2,5-dioxoimidazolidin-4-yl]methyl}-2H-1,2,3-triazole-4-carboxamide

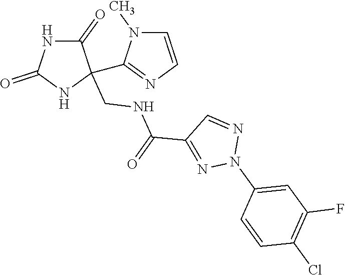 [SMILES: Cn1ccnc1C1(CNC(=O)c2cnn(-c3ccc(Cl)c(F)c3)n2)NC(=O)NC1=O]

To a solution of ent-5-(aminomethyl)-5-(1-methyl-1H-imidazol-2-yl)imidazolidine-2,4-dione hydrochloride (70.0 mg, 285 μmol) and 2-(4-chloro-3-fluorophenyl)-2H-1,2,3-triazole-4-carboxylic acid (68.8 mg, 285 μmol) in DMF (1.75 ml) was added 1-ethyl-3-(3-dimethylaminopropyl)carbodiimide hydrochloride (71.0 mg, 370 μmol), 1-hydroxybenzotriazole hydrate (56.7 mg, 370 μmol) and N,N-diisopropylethylamine (800 μmol). The reaction mixture was stirred overnight at RT and concentrated under reduced pressure. The crude product was purified by preparative HPLC (method 3f). After lyophilisation, 64.2 mg (98% purity, 51% yield) of the desired product were obtained.

LC-MS (Method 8): Rₜ=0.76 min; MS (ESIpos): m/z=433 [M+H]⁺

¹H-NMR (400 MHz, DMSO-d6) δ [ppm]: −0.008 (0.72), 0.008 (0.74), 2.524 (0.41), 3.508 (16.00), 4.149 (0.47), 4.166 (0.52), 4.183 (1.69), 4.203 (2.10), 4.219 (1.71), 4.238 (0.54), 4.254 (0.46), 6.941 (3.81), 6.944 (4.01), 7.253 (3.81), 7.255 (3.97), 7.856 (0.95), 7.878 (1.99), 7.897 (1.97), 7.919 (1.88), 7.925 (1.77), 7.941 (0.82), 7.947 (0.88), 8.060 (1.42), 8.066 (1.32), 8.085 (1.40), 8.091 (1.31), 8.436 (3.97), 8.547 (5.64), 8.653 (0.79), 8.670 (1.68), 8.686 (0.80), 11.231 (0.62).

### Example 146

### ent-N-{[4-(1-methyl-1H-imidazol-2-yl)-2,5-dioxoimidazolidin-4-yl]methyl}-2-(3,4,5-trifluorophenyl)-2H-1,2,3-triazole-4-carboxamide

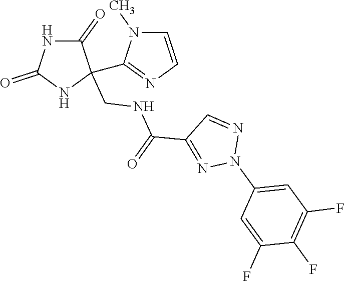 [SMILES: Cn1ccnc1C1(CNC(=O)c2cnn(-c3cc(F)c(F)c(F)c3)n2)NC(=O)NC1=O]

To a solution of ent-5-(aminomethyl)-5-(1-methyl-1H-imidazol-2-yl)imidazolidine-2,4-dione hydrochloride (1:1) (80.0 mg, 326 μmol) and 2-(3,4,5-trifluorophenyl)-2H-1,2,3-triazole-4-carboxylic acid (79.2 mg, 326 μmol) in DMF (2 ml) was added 1-ethyl-3-(3-dimethylaminopropyl)carbodiimide hydrochloride (81.2 mg, 423 μmol), 1-hydroxybenzotriazole hydrate (64.8 mg, 423 μmol) and N,N-diisopropylethylamine (912 μmol). The reaction mixture was stirred overnight at RT and concentrated under reduced pressure. The crude product was purified by preparative HPLC (method 3f). After lyophilisation, 74.8 mg (99% purity, 52% yield) of the desired product were obtained.

LC-MS (Method 8): Rₜ=0.75 min; MS (ESIpos): m/z=435 [M+H]⁺

¹H-NMR (400 MHz, DMSO-d6) δ [ppm]: −0.008 (1.24), 0.008 (1.26), 2.523 (0.58), 3.507 (16.00), 4.139 (0.67), 4.156 (0.75), 4.173 (1.51), 4.190 (1.35), 4.222 (1.37), 4.238 (1.51), 4.256 (0.74), 4.272 (0.67), 6.937 (3.81), 6.939 (3.97), 7.250 (3.83), 7.252 (3.91), 7.996 (1.50), 8.011 (1.78), 8.017 (1.82), 8.033 (1.65), 8.427 (3.74), 8.564 (5.66), 8.661 (0.79), 8.677 (1.69), 8.693 (0.78), 11.223 (0.93).

### Example 147

### ent-2-(3,4-difluorophenyl)-N-{[4-(1-methyl-1H-imidazol-2-yl)-2,5-dioxoimidazolidin-4-yl]methyl}2H-1,2,3-triazole-4-carboxamide

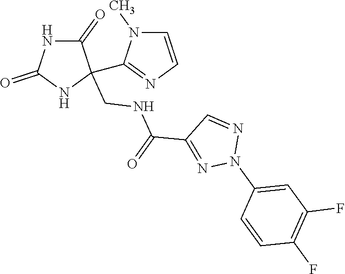 [SMILES: Cn1ccnc1C1(CNC(=O)c2cnn(-c3ccc(F)c(F)c3)n2)NC(=O)NC1=O]

To a solution of ent-5-(aminomethyl)-5-(1-methyl-1H-imidazol-2-yl)imidazolidine-2,4-dione hydrochloride (1:1) (80.0 mg, 326 μmol) and 2-(3,4-difluorophenyl)-2H-1,2,3-triazole-4-carboxylic acid (73.3 mg, 326 μmol) in DMF (2 ml) was added 1-ethyl-3-(3-dimethylaminopropyl)carbodiimide hydrochloride (81.2 mg, 423 μmol), 1-hydroxybenzotriazole hydrate (64.8 mg, 423 μmol) and N,N-diisopropylethylamine (160 μl, 910 μmol). The reaction mixture was stirred overnight at RT and concentrated under reduced pressure. The crude product was purified by preparative HPLC (method 3f). After lyophilisation, 79.1 mg (99% purity, 58% yield) of the desired product were obtained.

LC-MS (Method 8): Rₜ=0.70 min; MS (ESIpos): m/z=417 [M+H]⁺

¹H-NMR (400 MHz, DMSO-d6) δ [ppm]: −0.008 (0.91), 0.008 (0.86), 3.507 (16.00), 4.148 (0.42), 4.165 (0.48), 4.182 (1.72), 4.200 (2.92), 4.216 (1.76), 4.235 (0.50), 4.251 (0.42), 6.941 (3.95), 6.944 (4.06), 7.252 (3.95), 7.254 (4.03), 7.699 (0.57), 7.722 (1.25), 7.747 (1.28), 7.769 (0.73), 7.901 (1.00), 7.911 (0.67), 7.923 (0.80), 8.084 (0.64), 8.090 (0.63), 8.101 (0.73), 8.108 (0.79), 8.111 (0.82), 8.118 (0.68), 8.129 (0.69), 8.136 (0.64), 8.436 (3.80), 8.523 (6.12), 8.624 (0.82), 8.641 (1.69), 8.656 (0.80), 11.229 (1.08).

### Example 148

### ent-N-{[2,5-dioxo-4-(1,3-thiazol-4-yl)imidazolidin-4-yl]methyl}-2-(4-fluorophenyl)-2H-1,2,3-triazole-4-carboxamide

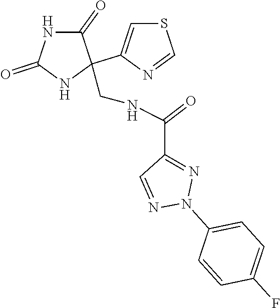 [SMILES: O=C1NC(=O)C(CNC(=O)c2cnn(-c3ccc(F)cc3)n2)(c2cscn2)N1]

To a solution of rac-5-(aminomethyl)-5-(1,3-thiazol-4-yl)imidazolidine-2,4-dione hydrochloride (67.0 mg, 269 μmol) and 2-(4-fluorophenyl)-2H-1,2,3-triazole-4-carboxylic acid (55.8 mg, 269 μmol) in DMF (1.7 ml) was added 1-ethyl-3-(3-dimethylaminopropyl)carbodiimide hydrochloride (67.1 mg, 350 μmol), 1-hydroxybenzotriazole hydrate (53.6 mg, 350 μmol) and N,N-diisopropylethylamine (230 μl, 1.3 mmol). The reaction mixture was stirred for 3 days at RT and concentrated under reduced pressure. The crude product was purified by preparative HPLC (method 3f). A second purification by preparative chiral HPLC [amount: 60 mg; column: Daicel Chiralpack IF 5 μm 250*20 mm; eluent:ethanol; flow rate: 15 ml/min; temperature: 60° C.; UV detection: 220 nm] gave 30.0 mg (96% purity, 27% yield) of the desired product.

Analytical chiral HPLC: Rₜ=8.16 min, e.e. =>99% [column: 250×4.6 mm filled with Daicel Chiralpack IF 5 μm; eluent:ethanol; flow rate: 1 ml/min; temperature: 60° C.; UV detection: 220 nm]

LC-MS (Method 7): Rₜ=1.23 min; MS (ESIpos): m/z=402 [M+H]⁺

¹H-NMR (400 MHz, DMSO-d6) δ [ppm]: −0.150 (0.46), −0.008 (3.93), 0.008 (4.16), 0.146 (0.45), 1.235 (0.71), 2.323 (0.41), 2.328 (0.57), 2.332 (0.41), 2.366 (0.45), 2.523 (2.54), 2.665 (0.49), 2.670 (0.68), 2.674 (0.50), 2.710 (0.52), 4.049 (1.33), 4.065 (1.47), 4.083 (3.57), 4.099 (3.30), 4.117 (3.31), 4.132 (3.68), 4.151 (1.53), 4.167 (1.33), 7.447 (0.48), 7.456 (5.35), 7.462 (1.87), 7.479 (8.98), 7.488 (1.09), 7.495 (1.93), 7.501 (5.92), 7.509 (0.68), 7.850 (9.41), 7.855 (9.52), 8.066 (0.56), 8.075 (5.47), 8.081 (2.21), 8.087 (5.92), 8.092 (3.45), 8.098 (5.81), 8.104 (2.11), 8.110 (5.42), 8.119 (0.62), 8.380 (9.13), 8.483 (16.00), 8.553 (1.82), 8.569 (3.97), 8.585 (1.83), 9.149 (6.57), 9.154 (6.78), 10.884 (2.06).

### Example 149

### diamix-N-{[4-(2,2-dimethylcyclopropyl)-2,5-dioxoimidazolidin-4-yl]methyl}-2-phenyl-2H-1,2,3-triazole-4-carboxamide

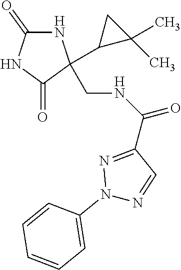 [SMILES: CC1(C)CC1C1(CNC(=O)c2cnn(-c3ccccc3)n2)NC(=O)NC1=O]

381 mg (1.99 mmol) of 1-(3-dimethylaminopropyl)-3-ethylcarbodiimide hydrochloride were added to a solution of 310 mg (1.33 mmol) of 5-(aminomethyl)-5-(2,2-dimethylcyclopropyl)imidazolidine-2,4-dione hydrochloride, 251 mg (1.33 mmol) of 2-phenyl-2H-1,2,3-triazole-4-carboxylic acid, 2.0 mL (2.00 mmol) of a 1N solution of 1-hydroxy-7-azabenzotriazole in N,N-dimethylformamide and 0.55 mL (4.00 mmol) of triethylamine in 5 mL of N,N-dimethylformamide and the reaction was allowed to stir at room temperature for 16 hours. The solvent was evaporated and the residue was dissolved in dichloromethane, washed with saturated sodium bicarbonate solution (aq.) and 2N hydrocloric acid (aq.). The organic extract was separated, dried over magnesium sulfate, filtered and concentrated to afford a residue, which was purified by flash column chromatography on silica gel eluting with dichloromethane/methanol mixtures to give 230 mg (47%, 85% purity) of the product as an off-white sticky solid.

¹H-NMR (300 MHz, DMSO-d₆): δ [ppm]=0.35-0.51 (m, 2H), 0.84-1.12 (m, 7H), 3.53-3.81 (m, 2H), 7.53 (t, 1H), 7.56-7.65 (m, 3H), 8.07 (d, 2H), 8.43-8.55 (m, 2H), 10.76 (bp, 1H).

LC-MS (Method 1): Rₜ=2.911 min. MS (Mass method 1): m/z=369 (M+H)⁺

### Example 150

### ent-N-({4-[rac-2,2-dimethylcyclopropyl]-2,5-dioxoimidazolidin-4-yl}methyl)-2-phenyl-2H-1,2,3-triazole-4-carboxamide

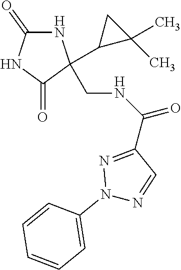 [SMILES: CC1(C)CC1C1(CNC(=O)c2cnn(-c3ccccc3)n2)NC(=O)NC1=O]

Enantiomeric separation of diamix-N-{[4-(2,2-dimethylcyclopropyl)-2,5-dioxoimidazolidin-4-yl]methyl}-2-phenyl-2H-1,2,3-triazole-4-carboxamide (228.6 mg) by preparative chiral HPLC [column: AD-H 5 u 250×20 mmO; flow rate: 70 ml/min; temperature: 40° C.; UV detection: 210 nm] afforded 7.30 mg of the desired product.

Analytical chiral HPLC: Rₜ=3.809 min, e.e. =>99% [column: Daicel AD; eluent: CO₂:85% Isopropanol 15%; flow rate: 3 ml/min; UV detection: 210 nm]

¹H-NMR (400 MHz, DMSO-d6) δ [ppm]: −0.008 (1.69), 0.008 (1.33), 0.449 (1.10), 0.461 (1.46), 0.472 (1.35), 0.483 (1.38), 0.593 (1.27), 0.606 (1.85), 0.619 (1.24), 0.867 (1.49), 0.882 (1.60), 0.889 (1.61), 0.900 (1.88), 0.974 (15.40), 1.011 (16.00), 2.524 (0.57), 3.567 (1.00), 3.582 (1.14), 3.601 (1.57), 3.616 (1.41), 3.728 (1.46), 3.745 (1.57), 3.761 (1.10), 3.779 (1.03), 7.488 (0.98), 7.506 (2.55), 7.525 (1.77), 7.608 (3.16), 7.629 (4.49), 7.643 (1.08), 7.648 (2.44), 7.725 (3.35), 8.062 (3.94), 8.064 (4.23), 8.083 (4.24), 8.086 (3.15), 8.469 (7.65), 8.477 (0.44), 8.494 (0.94), 8.510 (1.78), 8.526 (0.92), 10.633 (2.68).

LC-MS (Method 7): Rₜ=0.82 min; MS (ESIpos): m/z=369 [M+H]⁺

### Example 151

### 2-(2-chloro-4-fluorophenyl)-N-{[(4R)-4-cyclopropyl-2,5-dioxoimidazolidin-4-yl]methyl}-2H-1,2,3-triazole-4-carboxamide

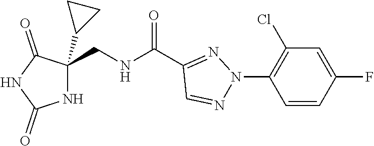 [SMILES: O=C1NC(=O)[C@](CNC(=O)c2cnn(-c3ccc(F)cc3Cl)n2)(C2CC2)N1]

2-(2-chloro-4-fluorophenyl)-2H-1,2,3-triazole-4-carboxylic acid (20.7 mg, 96% purity, 82.4 μmol) dissolved in 2 ml of DMF was treated with N-ethyl-N-isopropylpropan-2-amine (40 μl, 230 μmol), 3-{[(ethylimino)methylene]amino}-N,N-dimethylpropan-1-amine hydrochloride (20.5 mg, 107 μmol), 1H-benzotriazol-1-ol hydrate (16.4 mg, 107 μmol) and (5R)-5-(aminomethyl)-5-cyclopropylimidazolidine-2,4-dione hydrochloride (16.9 mg, 82.4 μmol). The mixture was stirred over night at room temperature. The product was purified by preparative HPLC (Instrument: Waters Prep LC/MS System, Column: Phenomenex Kinetex C18 5 μm 100×30 mm; eluent A: water, eluent B: acetonitrile, eluent C: 2% formic acid in water, eluent D: acetonitrile/water (80 Vol. %/20 Vol. %), flow: 80 ml/min; room temperature, UV-detection: 200-400 nm, At-Column Injektion; gradient: eluent A 0 bis 2 min 63 ml, eluent B 0 to 2 min 7 ml, eluent A 2 to 10 min from 63 ml to 39 ml and eluent B from 7 ml to 31 ml, 10 to 12 min 0 ml eluent A and 70 ml eluent B. Eluent C and eluent D constant flow 5 ml/min over the whole runtime). After lyophilization, 12.8 mg (100% purity, 40% yield) of the title compound were obtained.

LC-MS (Method 7): Rₜ=1.34 min; MS (ESIpos): m/z=393 [M+H]⁺

¹H-NMR (400 MHz, DMSO-d6) δ [ppm]: −0.008 (0.44), 0.008 (0.48), 0.094 (0.46), 0.105 (0.90), 0.113 (1.26), 0.127 (1.45), 0.137 (1.42), 0.144 (1.15), 0.155 (0.64), 0.316 (1.31), 0.328 (1.29), 0.334 (1.52), 0.343 (1.22), 0.354 (1.08), 0.365 (0.83), 0.399 (2.36), 0.416 (4.73), 0.435 (3.67), 0.452 (1.15), 1.115 (0.64), 1.133 (1.38), 1.149 (1.93), 1.165 (1.35), 1.183 (0.53), 2.328 (0.53), 2.670 (0.53), 3.660 (0.51), 3.679 (4.38), 3.683 (4.22), 3.695 (4.18), 3.699 (4.36), 3.718 (0.48), 7.483 (1.77), 7.490 (1.91), 7.505 (2.48), 7.512 (2.69), 7.525 (1.97), 7.532 (2.11), 7.580 (6.54), 7.824 (3.26), 7.831 (3.33), 7.846 (3.35), 7.853 (3.35), 7.865 (3.58), 7.879 (3.70), 7.887 (3.44), 7.901 (3.24), 8.434 (1.49), 8.450 (3.19), 8.466 (1.49), 8.506 (16.00), 10.640 (4.59).

### Example 152

### rac-2-(4-fluorophenyl)-N-[(4-methyl-2,5-dioxoimidazolidin-4-yl)methyl]-2H-1,2,3-triazole-4-carboxamide

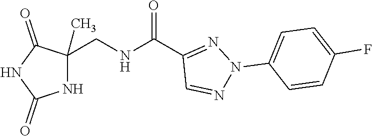 [SMILES: CC1(CNC(=O)c2cnn(-c3ccc(F)cc3)n2)NC(=O)NC1=O]

2-(4-fluorophenyl)-2H-1,2,3-triazole-4-carboxylic acid (115 mg, 557 μmol) dissolved in 5 ml of DMF was treated with N-ethyl-N-isopropylpropan-2-amine (270 μl, 1.6 mmol), 3-{[(ethylimino)methylene]amino}-N,N-dimethylpropan-1-amine hydrochloride (139 mg, 724 μmol), 1H-benzotriazol-1-ol hydrate (111 mg, 724 μmol) and rac-5-(aminomethyl)-5-methylimidazolidine-2,4-dione hydrochloride (100 mg, 557 μmol). The mixture was stirred over night at room temperature. The product was purified by preparative HPLC (Instrument: Waters Prep LC/MS System, Column: Phenomenex Kinetex C18 5 μm 100×30 mm; eluent A: water, eluent B: acetonitrile, eluent C: 2% formic acid in water, eluent D: acetonitrile/water (80 Vol. %/20 Vol. %); flow: 80 ml/min; room temperature, UV-detection: 200-400 nm, At-Column Injektion; gradient: eluent A 0 bis 2 min 63 ml, eluent B 0 to 2 min 7 ml, eluent A 2 to 10 min from 63 ml to 39 ml and eluent B from 7 ml to 31 ml, 10 to 12 min 0 ml eluent A and 70 ml eluent B. Eluent C and eluent D constant flow 5 ml/min over the whole runtime). After lyophilization, 86.0 mg (100% purity, 46% yield) and 71.0 mg (98% purity, 37% yield) of the title compound were obtained.

LC-MS (Method 7): Rₜ=1.15 min; MS (ESIpos): m/z=333 [M+H]⁺

¹H-NMR (400 MHz, DMSO-d6) δ [ppm]: 0.008 (0.61), 1.292 (16.00), 2.074 (0.56), 3.479 (0.70), 3.495 (0.79), 3.513 (1.89), 3.529 (1.73), 3.549 (1.77), 3.565 (1.89), 3.582 (0.78), 3.599 (0.74), 7.460 (2.54), 7.466 (0.90), 7.482 (4.65), 7.499 (0.94), 7.504 (2.73), 7.868 (2.92), 8.087 (2.75), 8.099 (2.91), 8.105 (1.71), 8.110 (2.80), 8.117 (1.08), 8.122 (2.64), 8.477 (7.49), 8.536 (0.86), 8.552 (1.80), 8.568 (0.86), 10.672 (2.34).

### Example 153

### ent-2-(4-fluorophenyl)-N-[(4-methyl-2,5-dioxoimidazolidin-4-yl)methyl]-2H-1,2,3-triazole-4-carboxamide

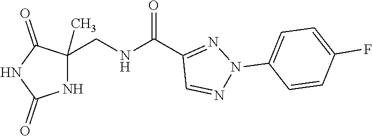 [SMILES: CC1(CNC(=O)c2cnn(-c3ccc(F)cc3)n2)NC(=O)NC1=O]

Enantiomeric separation of rac-2-(4-fluorophenyl)-N-[(4-methyl-2,5-dioxoimidazolidin-4-yl)methyl]-2H-1,2,3-triazole-4-carboxamide (157 mg, 472 μmol) was done using the following method.
Diacel Chiralcel AD SFC 250×20 mm
Eluent A: 70% CO₂, eluent B: 30% methanol
Flow: 80 ml/min
UV-detection: 210 nm
Temperature: 40° C.

After lyophilisation 34.0 mg (100% purity, 22% yield) of the title compound were obtained.

Chiral-HPLC: (Column: SFC; Diacel AD, eluent A: 70% CO₂, eluent B: 30% methanol, flow: 3 ml/min, UV-detection: 210 nm): Rₜ=1.781 min; 100% ee

LC-MS (Method 7): Rₜ=1.17 min; MS (ESIpos): m/z=333 [M+H]⁺

¹H-NMR (600 MHz, DMSO-d6) δ [ppm]: 1.294 (16.00), 2.071 (0.46), 3.167 (0.94), 3.175 (0.98), 3.494 (0.85), 3.505 (0.93), 3.517 (1.77), 3.527 (1.61), 3.555 (1.69), 3.566 (1.80), 3.578 (0.94), 3.589 (0.87), 7.463 (2.54), 7.467 (0.90), 7.478 (4.22), 7.489 (0.88), 7.492 (2.63), 7.847 (3.40), 8.091 (2.62), 8.095 (1.08), 8.099 (2.72), 8.103 (1.59), 8.106 (2.72), 8.111 (0.98), 8.114 (2.52), 8.469 (8.20), 8.514 (0.84), 8.525 (1.66), 8.535 (0.80), 10.654 (2.08).

### Example 154

### rac-N-{[4-(1-fluorocyclopropyl)-2,5-dioxoimidazolidin-4-yl]methyl}-2-(4-fluorophenyl)-2H-1,2,3-triazole-4-carboxamide

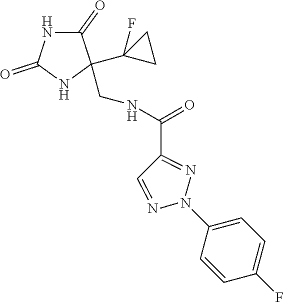 [SMILES: O=C1NC(=O)C(CNC(=O)c2cnn(-c3ccc(F)cc3)n2)(C2(F)CC2)N1]

To a solution of rac-5-(aminomethyl)-5-(1-fluorocyclopropyl)imidazolidine-2,4-dione hydrochloride (100 mg, 447 μmol) and 2-(4-fluorophenyl)-2H-1,2,3-triazole-4-carboxylic acid (92.6 mg, 447 μmol) in DMF (2.2 ml) was added 1-ethyl-3-(3-dimethylaminopropyl)carbodiimide hydrochloride (111 mg, 581 μmol), 1-hydroxybenzotriazole hydrate (89.0 mg, 581 μmol) and N,N-diisopropylethylamine (390 μl, 2.2 mmol). The reaction mixture was stirred overnight at RT, after which extra portion of 2-(4-fluorophenyl)-2H-1,2,3-triazole-4-carboxylic acid (8.57 mg, 44.7 μmol) and N,N-diisopropylethylamine (39 μl, 220 μmol) were added due to incomplete conversion. After stirring for 2 h at 50° C., the reaction mixture was concentrated under reduced pressure and the crude product was purified by preparative HPLC (Method 3f). After lyophilization, 26.6 mg (95% purity, 15% yield) of the desired product were obtained.

LC-MS (Method 8): Rₜ=0.78 min; MS (ESIpos): m/z=377 [M+H]⁺

¹H-NMR (400 MHz, DMSO-d₆) δ[ppm]: −0.149 (0.92), −0.024 (0.44), −0.021 (0.52), −0.019 (0.60), −0.016 (0.84), −0.014 (1.12), −0.011 (1.76), −0.008 (7.28), −0.007 (4.84), 0.006 (5.52), 0.008 (8.04), 0.011 (2.36), 0.013 (1.52), 0.016 (1.16), 0.018 (0.88), 0.021 (0.64), 0.023 (0.52), 0.025 (0.52), 0.027 (0.44), 0.146 (0.88), 0.866 (0.52), 0.889 (1.96), 0.920 (3.16), 0.946 (2.52), 0.980 (0.76), 0.989 (0.84), 1.024 (3.68), 1.030 (2.28), 1.074 (3.72), 1.080 (2.68), 1.194 (0.40), 1.217 (0.60), 1.235 (0.76), 2.073 (0.88), 2.366 (1.28), 2.391 (0.40), 2.517 (5.40), 2.521 (4.08), 2.522 (4.12), 2.524 (3.72), 2.557 (1.96), 2.560 (1.56), 2.562 (1.24), 2.565 (1.08), 2.567 (0.92), 2.569 (0.72), 2.572 (0.64), 2.574 (0.52), 2.577 (0.44), 2.710 (1.28), 3.716 (0.84), 3.732 (0.96), 3.750 (3.20), 3.767 (4.48), 3.785 (3.00), 3.804 (0.84), 3.820 (0.76), 7.452 (0.92), 7.461 (5.56), 7.466 (2.24), 7.483 (8.64), 7.493 (1.16), 7.499 (1.96), 7.505 (5.92), 7.514 (0.72), 8.068 (0.76), 8.077 (5.60), 8.083 (2.28), 8.089 (5.92), 8.094 (3.44), 8.100 (5.84), 8.106 (2.12), 8.112 (5.44), 8.121 (0.76), 8.144 (5.52), 8.474 (16.00), 8.533 (1.56), 8.549 (3.32), 8.565 (1.60), 10.865 (3.96).

### Example 155

### rac-N-{[4-(3,3-difluorocyclobutyl)-2,5-dioxoimidazolidin-4-yl]methyl}-2-(4-fluorophenyl)-2H-1,2,3-triazole-4-carboxamide

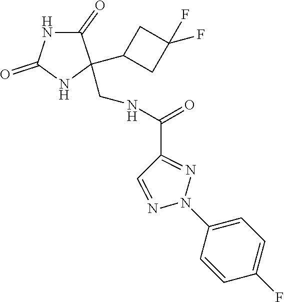 [SMILES: O=C1NC(=O)C(CNC(=O)c2cnn(-c3ccc(F)cc3)n2)(C2CC(F)(F)C2)N1]

To a solution of rac-5-(aminomethyl)-5-(3,3-difluorocyclobutyl)imidazolidine-2,4-dione hydrochloride (100 mg, 391 μmol) and 2-(4-fluorophenyl)-2H-1,2,3-triazole-4-carboxylic acid (81.0 mg, 391 μmol) in DMF (1.9 ml) was added 1-ethyl-3-(3-dimethylaminopropyl)carbodiimide hydrochloride (97.5 mg, 509 μmol), 1-hydroxybenzotriazole hydrate (77.9 mg, 509 μmol) and N,N-diisopropylethylamine (340 μl, 2.0 mmol). The reaction mixture was stirred overnight at RT and concentrated under reduced pressure. The crude product was purified by preparative HPLC (Method 3f). After lyophilisation, 32.1 mg (95% purity, 19% yield) of the desired product were obtained.

LC-MS (Method 7): Rₜ=1.44 min; MS (ESIneg): m/z=407 [M−H]⁻

¹H-NMR (600 MHz, DMSO-d6) δ [ppm]: 2.327 (0.67), 2.341 (1.02), 2.361 (1.00), 2.374 (0.81), 2.474 (1.36), 2.568 (2.42), 2.581 (3.08), 2.595 (2.10), 2.613 (1.49), 2.623 (1.32), 2.639 (1.16), 2.649 (1.09), 3.266 (0.59), 3.568 (9.46), 3.579 (9.50), 7.468 (5.58), 7.483 (9.89), 7.497 (5.63), 8.086 (5.88), 8.094 (5.89), 8.097 (3.61), 8.101 (5.86), 8.109 (5.68), 8.121 (8.86), 8.469 (16.00), 8.549 (2.09), 8.560 (4.32), 8.571 (2.03), 10.856 (2.74).

### Example 156

### rac-N-({2,5-dioxo-4-[3-(trifluoromethyl)pyridin-2-yl]imidazolidin-4-yl}methyl)-2-(4-fluorophenyl)-2H-1,2,3-triazole-4-carboxamide

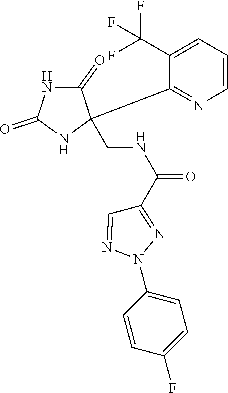 [SMILES: O=C1NC(=O)C(CNC(=O)c2cnn(-c3ccc(F)cc3)n2)(c2ncccc2C(F)(F)F)N1]

To a solution of rac-5-(aminomethyl)-5-[3-(trifluoromethyl)pyridin-2-yl]imidazolidine-2,4-dione hydrochloride (100 mg, 322 μmol) and 2-(4-fluorophenyl)-2H-1,2,3-triazole-4-carboxylic acid (75.6 mg, 365 μmol) in DMF (1.8 ml) was added 1-ethyl-3-(3-dimethylaminopropyl)carbodiimide hydrochloride (90.9 mg, 474 μmol), 1-hydroxybenzotriazole hydrate (72.6 mg, 474 μmol) and N,N-diisopropylethylamine (320 μl, 1.8 mmol). The reaction mixture was stirred overnight at RT and concentrated under reduced pressure. The crude product was purified by preparative HPLC (Method 3f). After lyophilisation, 50.3 mg (98% purity, 33% yield) of the desired product were obtained.

LC-MS (Method 7): Rₜ=1.48 min; MS (ESIpos): m/z=464 [M+H]⁺

¹H-NMR (600 MHz, DMSO-d6) δ [ppm]: 2.572 (0.61), 3.329 (0.74), 3.916 (0.48), 4.163 (2.80), 4.173 (3.03), 4.185 (3.43), 4.196 (3.18), 4.446 (3.22), 4.457 (3.50), 4.469 (3.01), 4.480 (2.77), 7.459 (5.69), 7.474 (10.56), 7.488 (5.85), 7.721 (3.00), 7.730 (3.17), 7.735 (3.15), 7.743 (3.07), 8.080 (5.97), 8.088 (6.24), 8.091 (3.94), 8.095 (6.19), 8.103 (5.83), 8.215 (8.90), 8.322 (6.69), 8.334 (9.20), 8.344 (2.38), 8.470 (16.00), 8.975 (4.62), 8.982 (4.56), 11.007 (6.17).

### Example 157

### rac-N-{[4-(5-cyclopropyl-1,3-thiazol-4-yl)-2,5-dioxoimidazolidin-4-yl]methyl}-2-(4-fluorophenyl)-2H-1,2,3-triazole-4-carboxamide

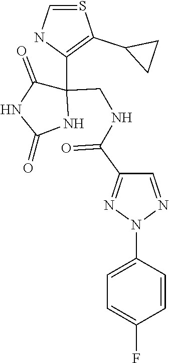

To a solution of rac-5-(aminomethyl)-5-(5-cyclopropyl-1,3-thiazol-4-yl)imidazolidine-2,4-dione hydrochloride (110 mg, 381 μmol) and 2-(4-fluorophenyl)-2H-1,2,3-triazole-4-carboxylic acid (78.9 mg, 381 μmol) in DMF (2 ml) was added 1-ethyl-3-(3-dimethylaminopropyl)carbodiimide hydrochloride (94.9 mg, 495 μmol), 1-hydroxybenzotriazole hydrate (75.8 mg, 495 μmol) and N,N-diisopropylethylamine (460 μl, 2.7 mmol). The reaction mixture was stirred overnight at RT and concentrated under reduced pressure. The crude product was purified by preparative HPLC (Method 3f). After lyophilisation, 70.1 mg (100% purity, 42% yield) of the desired product were obtained.

LC-MS (Method 8): Rₜ=0.80 min; MS (ESIpos): m/z=442 [M+H]⁺

¹H-NMR (400 MHz, DMSO-d6) δ [ppm]: −0.008 (1.00), 0.008 (1.05), 0.528 (0.68), 0.538 (0.98), 0.542 (1.27), 0.552 (1.48), 0.556 (1.72), 0.562 (1.35), 0.566 (1.85), 0.569 (1.61), 0.574 (1.40), 0.579 (1.24), 0.584 (0.98), 0.684 (0.92), 0.689 (1.22), 0.693 (1.35), 0.699 (1.66), 0.702 (1.79), 0.706 (1.46), 0.712 (1.59), 0.716 (1.53), 0.726 (1.49), 0.729 (1.07), 0.740 (0.78), 0.977 (0.55), 0.986 (0.52), 0.990 (0.44), 0.999 (1.53), 1.009 (1.99), 1.013 (1.83), 1.019 (4.15), 1.024 (2.23), 1.029 (2.64), 1.034 (2.18), 1.039 (4.39), 1.045 (1.99), 1.050 (1.83), 1.059 (1.48), 1.068 (0.50), 1.072 (0.46), 1.082 (0.50), 1.895 (0.85), 1.907 (1.70), 1.916 (1.81), 1.920 (1.07), 1.928 (3.28), 1.936 (1.11), 1.941 (1.70), 1.949 (1.59), 1.962 (0.72), 2.524 (0.79), 3.048 (0.41), 4.201 (0.59), 4.216 (0.78), 4.235 (3.93), 4.244 (4.17), 4.250 (4.06), 4.260 (3.97), 4.278 (0.72), 4.295 (0.65), 7.447 (0.55), 7.455 (5.26), 7.461 (1.83), 7.478 (8.29), 7.487 (1.00), 7.494 (1.86), 7.499 (5.63), 7.508 (0.59), 8.066 (0.61), 8.075 (5.31), 8.081 (2.12), 8.087 (5.61), 8.093 (3.27), 8.098 (5.61), 8.105 (1.97), 8.110 (5.13), 8.119 (0.54), 8.358 (6.18), 8.465 (1.72), 8.481 (3.78), 8.490 (16.00), 8.496 (1.96), 8.877 (14.17), 10.980 (4.67).

### Example 158

### rac-N-{[4-(2,5-dimethyl-1,3-thiazol-4-yl)-2,5-dioxoimidazolidin-4-yl]methyl}-2-(4-fluorophenyl)-2H-1,2,3-triazole-4-carboxamide

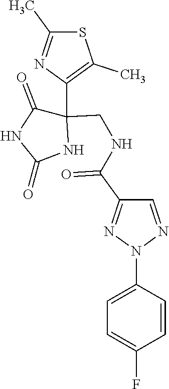 [SMILES: Cc1nc(C2(CNC(=O)c3cnn(-c4ccc(F)cc4)n3)NC(=O)NC2=O)c(C)s1]

To a solution of rac-5-(aminomethyl)-5-(2,5-dimethyl-1,3-thiazol-4-yl)imidazolidine-2,4-dione hydrochloride (60.0 mg, 217 μmol) and 2-(4-fluorophenyl)-2H-1,2,3-triazole-4-carboxylic acid (44.9 mg, 217 μmol) in DMF (1.1 ml) was added 1-ethyl-3-(3-dimethylaminopropyl)carbodiimide hydrochloride (54.0 mg, 282 μmol), 1-hydroxybenzotriazole hydrate (43.2 mg, 282 μmol) and N,N-diisopropylethylamine (260 μl, 1.5 mmol). The reaction mixture was stirred overnight at RT and concentrated under reduced pressure. The crude product was purified by preparative HPLC (Method 3f). After lyophilisation, 27.2 mg (98% purity, 29% yield) of the desired product were obtained.

LC-MS (Method 8): Rₜ=0.73 min; MS (ESIpos): m/z=430 [M+H]⁺

¹H-NMR (400 MHz, DMSO-d6) δ [ppm]: −0.008 (0.94), 0.008 (0.94), 2.392 (16.00), 2.524 (0.81), 3.980 (1.51), 3.994 (2.32), 4.009 (1.51), 7.465 (2.21), 7.470 (0.79), 7.487 (3.62), 7.503 (0.77), 7.508 (2.40), 8.073 (2.32), 8.078 (0.94), 8.085 (2.45), 8.090 (1.40), 8.096 (2.42), 8.102 (0.84), 8.107 (2.23), 8.478 (7.43), 8.548 (3.34), 8.655 (0.68), 8.671 (1.48), 8.686 (0.69), 11.126 (0.48).

### Example 159

### rac-N-{[4-(1-chlorocyclopropyl)-2,5-dioxoimidazolidin-4-yl]methyl}-2-(4-fluorophenyl)-2H-1,2,3-triazole-4-carboxamide

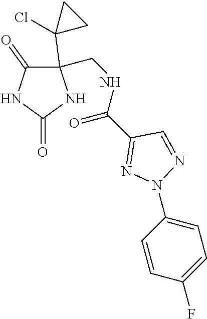 [SMILES: O=C1NC(=O)C(CNC(=O)c2cnn(-c3ccc(F)cc3)n2)(C2(Cl)CC2)N1]

To a solution of rac-5-(aminomethyl)-5-(1-chlorocyclopropyl)imidazolidine-2,4-dione hydrochloride (50.0 mg, 208 μmol) and 2-(4-fluorophenyl)-2H-1,2,3-triazole-4-carboxylic acid (47.5 mg, 229 μmol) in DMF (1.1 ml) was added 1-ethyl-3-(3-dimethylaminopropyl)carbodiimide hydrochloride (51.9 mg, 271 μmol), 1-hydroxybenzotriazole hydrate (41.5 mg, 271 μmol) and N,N-diisopropylethylamine (220 μl, 1.2 mmol). The reaction mixture was stirred overnight at RT and concentrated under reduced pressure. The crude product was purified by preparative HPLC (Method 3f). After lyophilisation, 12.9 mg (99% purity, 16% yield) of the desired product were obtained.

LC-MS (Method 7): Rₜ=1.43 min; MS (ESIpos): m/z=393 [M+H]⁺

¹H-NMR (400 MHz, DMSO-d6) δ [ppm]: −0.008 (1.95), −0.006 (1.52), 0.006 (1.61), 0.008 (2.12), 0.014 (0.42), 1.051 (0.93), 1.066 (1.86), 1.075 (4.06), 1.091 (5.59), 1.104 (2.20), 1.118 (1.19), 1.214 (1.27), 1.226 (2.12), 1.240 (4.99), 1.255 (3.64), 1.268 (1.78), 1.280 (1.10), 2.073 (2.20), 2.323 (0.68), 2.328 (0.93), 2.333 (0.68), 2.367 (0.59), 2.391 (0.59), 2.524 (3.72), 2.558 (1.02), 2.560 (0.85), 2.563 (0.68), 2.565 (0.59), 2.568 (0.51), 2.666 (0.76), 2.670 (1.02), 2.675 (0.76), 2.710 (0.59), 3.048 (1.02), 3.269 (0.42), 3.272 (0.42), 3.274 (0.51), 3.277 (0.51), 3.279 (0.68), 3.282 (0.76), 3.285 (1.10), 3.299 (5.08), 3.347 (1.52), 3.352 (1.10), 3.663 (1.61), 3.678 (1.78), 3.697 (2.88), 3.713 (2.71), 3.774 (2.79), 3.790 (3.05), 3.809 (1.86), 3.825 (1.69), 7.452 (0.85), 7.461 (5.33), 7.466 (1.95), 7.483 (8.72), 7.493 (1.10), 7.499 (1.95), 7.505 (5.84), 7.514 (0.68), 8.062 (0.76), 8.071 (5.50), 8.077 (2.46), 8.083 (6.52), 8.094 (11.43), 8.100 (3.13), 8.106 (5.67), 8.114 (0.76), 8.467 (16.00), 8.483 (3.72), 8.499 (1.69), 10.860 (4.32).

### Example 160

### 2-(4-chloro-2-fluorophenyl)-N-{[(4R)-4-cyclopropyl-2,5-dioxoimidazolidin-4-yl]methyl}-2H-1,2,3-triazole-4-carboxamide

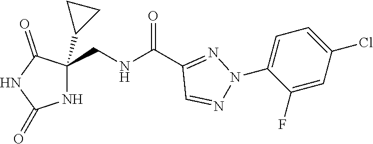 [SMILES: O=C1NC(=O)[C@](CNC(=O)c2cnn(-c3ccc(Cl)cc3F)n2)(C2CC2)N1]

2-(4-chloro-2-fluorophenyl)-2H-1,2,3-triazole-4-carboxylic acid (58.7 mg, 243 μmol) dissolved in 1.5 ml of DMF was treated with N-ethyl-N-isopropylpropan-2-amine (120 μl, 680 μmol), 3-{[(ethylimino)methylene]amino}-N,N-dimethylpropan-1-amine hydrochloride (60.6 mg, 316 μmol), 1H-benzotriazol-1-ol hydrate (48.4 mg, 316 μmol) and (5R)-5-(aminomethyl)-5-cyclopropylimidazolidine-2,4-dione hydrochloride (58.7 mg, 243 μmol). The mixture was stirred over night at room temperature. The product was purified by preparative HPLC (Instrument: Waters Prep LC/MS System, Column: Phenomenex Kinetex C18 5 μm 100×30 mm; eluent A: water, eluent B: acetonitrile, eluent C: 2% formic acid in water, eluent D: acetonitrile/water (80 Vol. %/20 Vol. %); flow: 80 ml/min; room temperature, UV-detection: 200-400 nm, At-Column Injektion; gradient: eluent A 0 bis 2 min 63 ml, eluent B 0 to 2 min 7 ml, eluent A 2 to 10 min from 63 ml to 39 ml and eluent B from 7 ml to 31 ml, 10 to 12 min 0 ml eluent A and 70 ml eluent B. Eluent C and eluent D constant flow 5 ml/min over the whole runtime). After lyophilization, 65.0 mg (100% purity, 68% yield) of the title compound were obtained.

LC-MS (Method 7): Rₜ=1.44 min; MS (ESIpos): m/z=393 [M+H]⁺

¹H-NMR (600 MHz, CHLOROFORM-d) δ [ppm]: 0.363 (0.53), 0.371 (1.27), 0.380 (1.89), 0.388 (2.18), 0.397 (1.87), 0.405 (0.86), 0.413 (0.68), 0.422 (1.21), 0.428 (1.47), 0.436 (2.03), 0.444 (1.84), 0.453 (2.02), 0.461 (2.60), 0.470 (2.55), 0.478 (2.10), 0.487 (1.47), 0.496 (0.49), 0.595 (0.87), 0.603 (1.45), 0.609 (1.83), 0.613 (1.47), 0.618 (2.28), 0.623 (1.32), 0.627 (1.41), 0.631 (1.02), 0.642 (0.54), 1.202 (0.90), 1.211 (1.77), 1.216 (1.93), 1.220 (1.35), 1.225 (3.03), 1.233 (1.63), 1.238 (1.49), 1.247 (0.65), 1.864 (1.08), 3.731 (2.45), 3.741 (2.65), 3.755 (3.04), 3.764 (2.54), 4.086 (2.81), 4.099 (2.91), 4.110 (2.59), 4.123 (2.44), 6.468 (3.64), 7.207 (2.86), 7.208 (2.99), 7.223 (3.33), 7.234 (3.78), 7.238 (2.58), 7.252 (3.60), 7.255 (3.11), 7.664 (2.92), 7.678 (4.83), 7.692 (2.70), 7.896 (1.56), 8.197 (16.00).

### Example 161

### N-{[(4R)-4-cyclopropyl-2,5-dioxoimidazolidin-4-yl]methyl}-2-(2,4-dichlorophenyl)-2H-1,2,3-triazole-4-carboxamide

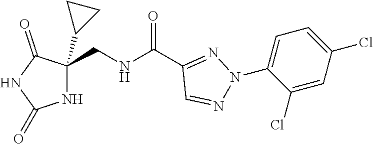 [SMILES: O=C1NC(=O)[C@](CNC(=O)c2cnn(-c3ccc(Cl)cc3Cl)n2)(C2CC2)N1]

2-(2,4-dichlorophenyl)-2H-1,2,3-triazole-4-carboxylic acid (62.7 mg, 243 μmol) dissolved in 1.5 ml of DMF was treated with N-ethyl-N-isopropylpropan-2-amine (120 μl, 680 μmol), 3-{[(ethylimino)methylene]amino}-N,N-dimethylpropan-1-amine hydrochloride (60.6 mg, 316 μmol), 1H-benzotriazol-1-ol hydrate (48.4 mg, 316 μmol) and (5R)-5-(aminomethyl)-5-cyclopropylimidazolidine-2,4-dione hydrochloride (50.0 mg, 243 μmol). The mixture was stirred over night at room temperature. The product was purified by preparative HPLC (Instrument: Waters Prep LC/MS System, Column: Phenomenex Kinetex C18 5 μm 100×30 mm; eluent A: water, eluent B: acetonitrile, eluent C: 2% formic acid in water, eluent D: acetonitrile/water (80 Vol. %/20 Vol. %); flow: 80 ml/min; room temperature, UV-detection: 200-400 nm, At-Column Injektion; gradient: eluent A 0 bis 2 min 63 ml, eluent B 0 to 2 min 7 ml, eluent A 2 to 10 min from 63 ml to 39 ml and eluent B from 7 ml to 31 ml, 10 to 12 min 0 ml eluent A and 70 ml eluent B. Eluent C and eluent D constant flow 5 ml/min over the whole runtime). After lyophilization, 64.0 mg (100% purity, 64% yield) of the title compound were obtained.

LC-MS (Method 7): Rₜ=1.52 min; MS (ESIpos): m/z=409 [M+H]⁺

¹H-NMR (600 MHz, CHLOROFORM-d) δ [ppm]: 0.380 (0.43), 0.387 (0.89), 0.396 (1.77), 0.405 (2.36), 0.411 (1.36), 0.414 (1.60), 0.421 (0.97), 0.430 (1.77), 0.439 (3.61), 0.444 (3.33), 0.450 (4.71), 0.457 (2.79), 0.459 (2.60), 0.465 (1.35), 0.584 (0.55), 0.597 (1.51), 0.602 (1.38), 0.604 (1.32), 0.608 (1.48), 0.612 (1.84), 0.618 (1.69), 0.626 (0.92), 0.631 (0.54), 1.194 (0.81), 1.203 (1.48), 1.207 (1.59), 1.212 (1.23), 1.217 (2.88), 1.221 (1.18), 1.226 (1.45), 1.229 (1.32), 1.239 (0.65), 2.002 (0.47), 2.638 (15.90), 3.747 (2.30), 3.757 (2.53), 3.771 (2.92), 3.781 (2.55), 4.047 (2.64), 4.060 (2.76), 4.071 (2.43), 4.083 (2.31), 6.416 (3.14), 7.367 (3.87), 7.371 (3.81), 7.381 (4.68), 7.385 (4.47), 7.397 (0.67), 7.401 (0.60), 7.411 (0.73), 7.415 (0.68), 7.515 (7.99), 7.530 (6.70), 7.560 (8.03), 7.563 (7.19), 7.588 (1.43), 7.596 (1.68), 7.599 (2.03), 7.602 (2.09), 7.613 (1.70), 8.242 (16.00), 8.302 (2.27), 8.695 (0.86).

### Example 162

### rac-N-[(4-ethyl-2,5-dioxoimidazolidin-4-yl)methyl]-2-(4-fluorophenyl)-2H-1,2,3-triazole-4-carboxamide

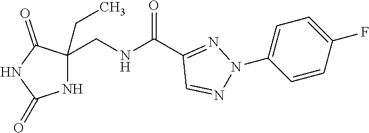 [SMILES: CCC1(CNC(=O)c2cnn(-c3ccc(F)cc3)n2)NC(=O)NC1=O]

2-(4-fluorophenyl)-2H-1,2,3-triazole-4-carboxylic acid (53.5 mg, 258 μmol) dissolved in 2 ml of DMF was treated with N-ethyl-N-isopropylpropan-2-amine (130 μl, 720 μmol), 3-{[(ethylimino)methylene]amino}-N,N-dimethylpropan-1-amine hydrochloride (64.4 mg, 336 μmol), 1H-benzotriazol-1-ol hydrate (51.4 mg, 336 μmol) and rac-5-(aminomethyl)-5-ethylimidazolidine-2,4-dione hydrochloride (50.0 mg, 258 μmol). The mixture was stirred over night at room temperature. The product was purified by preparative HPLC (Instrument: Waters Prep LC/MS System, Column: Phenomenex Kinetex C18 5 μm 100×30 mm; eluent A: water, eluent B: acetonitrile, eluent C: 2% formic acid in water, eluent D: acetonitrile/water (80 Vol. %/20 Vol. %); flow: 80 ml/min, room temperature, UV-detection: 200-400 nm, At-Column Injektion; gradient: eluent A 0 bis 2 min 63 ml, eluent B 0 to 2 min 7 ml, eluent A 2 to 10 min from 63 ml to 39 ml and eluent B from 7 ml to 31 ml, 10 to 12 min 0 ml eluent A and 70 ml eluent B. Eluent C and eluent D constant flow 5 ml/min over the whole runtime). After lyophilization, 66.0 mg (100% purity, 74% yield) of the title compound were obtained.

LC-MS (Method 7): Rₜ=1.23 min; MS (ESIpos): m/z=347 [M+H]⁺

¹H-NMR (400 MHz, DMSO-d6) δ [ppm]: −0.008 (0.52), 0.008 (0.54), 0.767 (6.30), 0.785 (15.08), 0.804 (6.90), 1.595 (1.10), 1.613 (1.66), 1.630 (2.55), 1.648 (2.17), 1.665 (1.14), 1.683 (2.35), 1.701 (2.62), 1.718 (1.73), 1.736 (1.10), 2.074 (1.42), 3.504 (1.01), 3.520 (1.18), 3.538 (3.66), 3.554 (3.84), 3.560 (3.85), 3.577 (3.58), 3.594 (1.14), 3.611 (1.04), 7.451 (0.52), 7.460 (5.27), 7.465 (1.85), 7.482 (8.97), 7.491 (1.02), 7.498 (1.88), 7.504 (5.79), 7.512 (0.61), 7.766 (5.54), 8.075 (0.58), 8.084 (5.45), 8.089 (2.22), 8.096 (5.79), 8.101 (3.36), 8.107 (5.73), 8.113 (2.05), 8.119 (5.33), 8.128 (0.55), 8.470 (16.00), 8.502 (1.66), 8.518 (3.46), 8.534 (1.62), 10.686 (4.45).

### Experimental Section—Evaluation of Pharmacological Activity

### Example B1: Production of Human ADAMTS-7

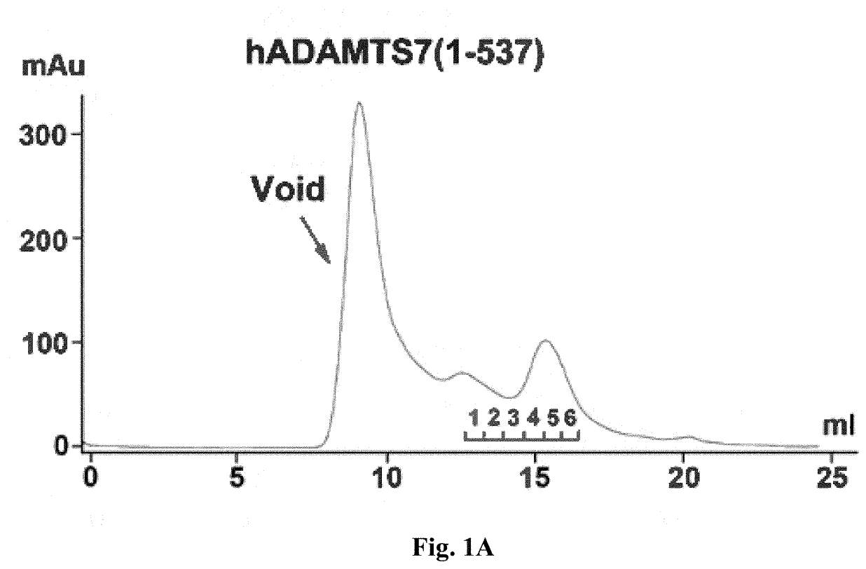

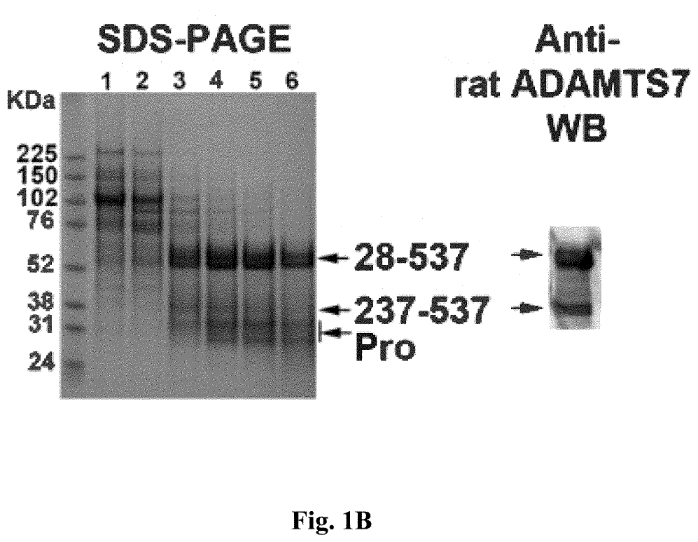

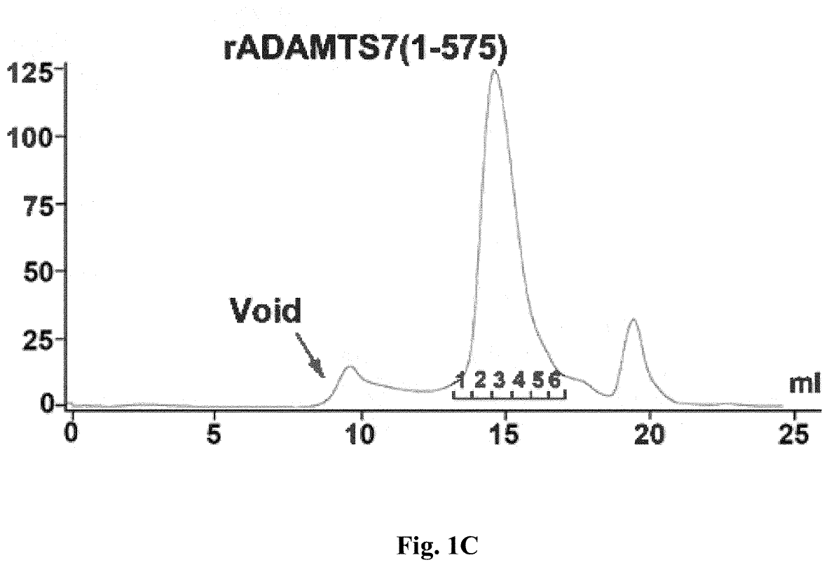

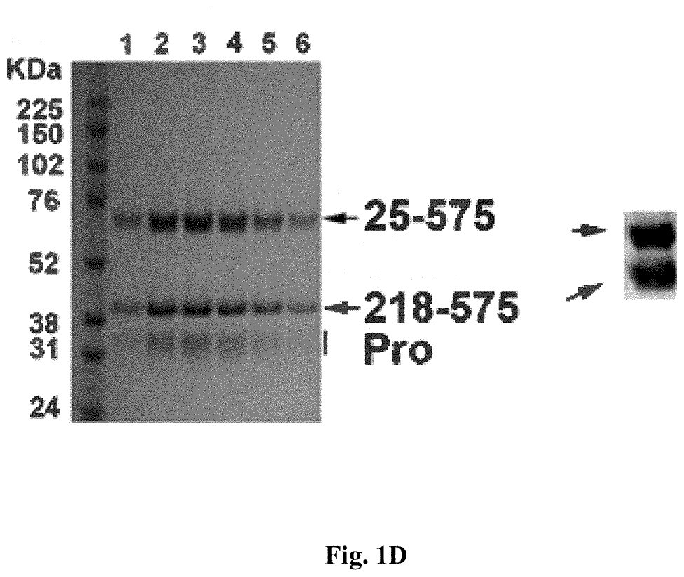

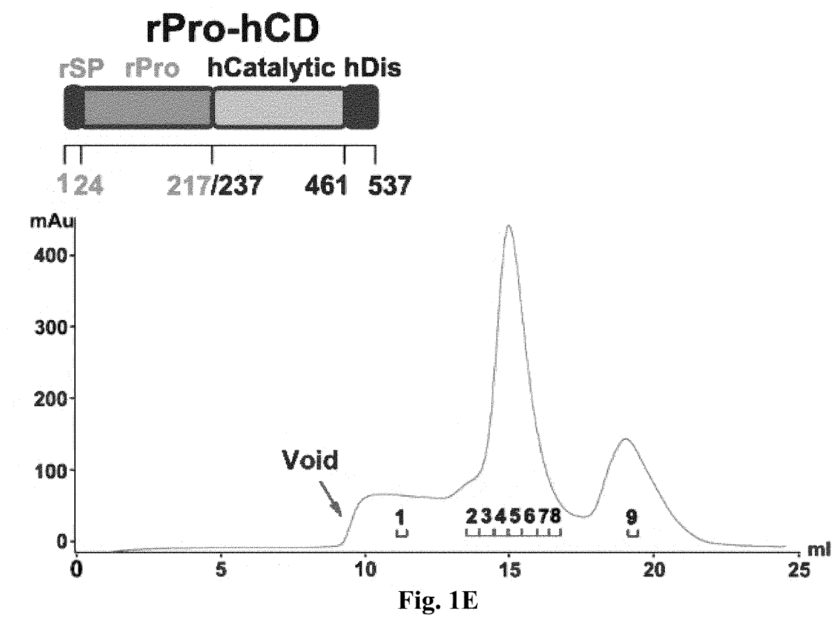

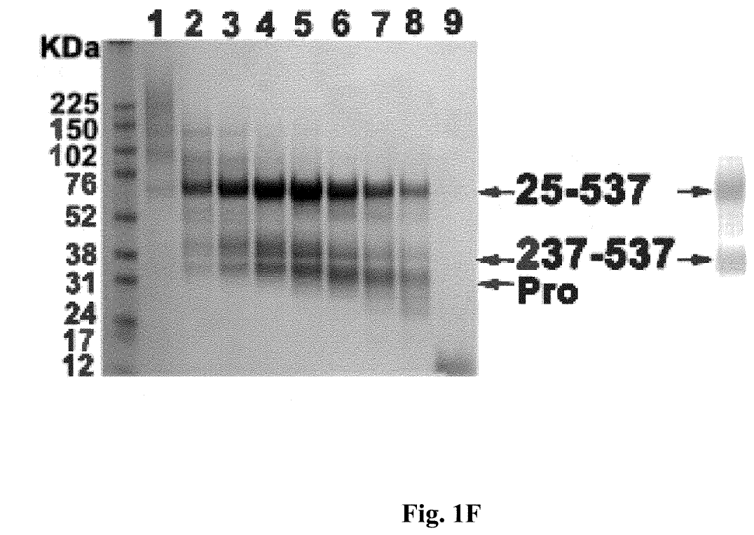

To study the activity of human ADAMTS-7 (hADAMTS-7), the inventors generated multiple constructs for the production of active ADAMTS-7 in E coli cells. These constructs contain the catalytic domain alone or catalytic domain with the Prodomain (Pro), Disintegrin domain (Dis), or TSR1 (FIG. 2 panel A). Different tags such as 6×His, GST, MBP, SUMO or Trigger factor (TF) were incorporated to the N-terminus of the constructs to improve the solubility of the protein and to facilitate protein purification, but none of these constructs produced soluble and active ADAMTS-7 to support further studies. Therefore, we tested catalytic domain containing constructs along with secretion signals in Expi293 mammalian cells. ADAMTS-7 proteins secreted into culture media were captured by affinity column and analyzed by analytical size exclusion column (SEC). A construct containing the secretion signal peptide (SP), Pro and CD domains of hADAMTS-7 (residues 1-537, now referred to as “hPro-hCD” in light of the later discovered hybrids in which the prodomain can be from a non-human species) with an affinity tag at the C-terminus was eluted largely in the void volume from SEC and yielded ˜0.2 mg/L of soluble ADAMTS-7 proteins in the elution fractions 3-6 (FIG. 1 panel A). Based on the size and N-terminal sequencing results of the bands on the SDS-page, the soluble ADAMTS-7 is confirmed to be a mixture of amino acid (aa) 28-537 (hPro-hCD, unprocessed ADAMTS-7), aa 237-537 (hCD domain only, furin processed ADAMTS-7), and fragments from Pro. The mixture was dominated by the unprocessed ADAMTS-7, which accounts for ˜90% of the population. A construct containing the secretion SP, an affinity/solubility tag and only hADAMTS-7 CD domain (residues 237-537) yielded mostly soluble aggregates (void). Soluble ADAMTS-7 proteins were only detectible by western blot (fractions B8-B12) (FIG. 3 panel A). Another construct that ends at TSR1 (hSP-hPro-hCD-hTSR1, residues 1-593), instead failed to overexpress soluble ADAMTS-7 proteins. Neither SDS-PAGE nor western blot can detect significant ADAMTS-7 proteins (FIG. 3 panel B). In comparison, rat ADAMTS-7 (rADAMTS-7) residues 1-575 (rSP-rPro-rCD-rTSR1) produced ˜0.6 mg/L soluble proteins (fractions1-6) from Expi293 cells with negligible aggregates (void) eluted from SEC. Purified rat ADAMTS-7 contains ˜1:1 molar ratio of unprocessed (aa 25-575) and processed (aa 218-575) ADAMTS-7, and some fragments from Pro (FIG. 1 panel B).

### Example B2: Hybrid Molecules to Improve the Production of Soluble hADAMTS-7

To improve the production of hADAMTS-7 protein, we also explored various other options. For example, we compared and analyzed the sequences of rat and human ADAMTS-7 in the Pro and CD domains (FIG. 2 panel B). CD domain sequence is well conserved. The amino acid sequence in the CD domain of rat and human is 84% identical (97% similar), compared to Prodomain which is 70% identical (89% similar). The rest of the sequences are different (i.e., neither identical nor similar) between the two species. Specifically, the CD domain has only 10 different residues between rat and human over 302 residues, while Pro has 22 different ones over 202 residues (FIG. 2 panel B). To test whether rat Pro played a role in facilitating protein folding and yielding more soluble ADAMTS-7 proteins, we designed hybrid molecules of rat SP-Pro (1-217) followed by human CD (237-537), named rPro-hCD (FIG. 1 panel C). The hybrid molecule was purified by Ni affinity column and analyzed by SEC as done to hADAMTS-7 (1-537). Little was eluted as aggregates in the void volume preceding the soluble peak (fractions 2-8). The soluble proteins were analyzed as a mixture of the unprocessed (aa 25-537) and processed (aa 237-537) ADAMTS-7, and Pro (FIG. 1 panel C). The yield of ADAMTS-7 protein after affinity and SEC two-step purification was 2.2 mg/L for rPro-hCD. This is in contrast with ˜0.2 mg/L yield of the hPro-hCD, namely hADAMTS-7 (1-537). Our result suggests that the rat Pro is more effective in driving effective folding of the CD domain, therefore improving the yield of the soluble ADAMTS-7 proteins ˜10 fold. Therefore, this example demonstrates a solution for the problem of protein solubility as well as for the problem of expression yield, both of which were lower for the fully human construct (hPro-hCD) as compared to the hybrid construct (rPro-hCD).

### Example B3: Engineering Furin Cleavage Site to Manipulate the Production Ratio of the Processed and Unprocessed ADAMTS-7

We hypothesized multiple furin cleavage sites in rat Pro (FIG. 4 panel A), one after residue R58 or R60 (RVLR⁵⁸↓KR⁶⁰↓D) of Pro, and another between Pro and CD domains after R217 (RQQR²¹⁷↓S). These cleavage sites were found conserved in rat, mouse and human Pro. Sequential cleavage or processing at these furin sites by cellular furin enzyme leading to a complete removal of the Pro domain from the rest of the protein is likely a necessary step to a fully active or mature ADAMTS-7. Recombinant production of hPro-hCD and rPro-hCD revealed an inefficient furin processing in the mammalian cell culture (FIG. 1 panels A and C). Both constructs yielded a mixture of processed and unprocessed ADAMTS-7. We hypothesized that this could be attributed to a less sufficient amount of endogenous furin produced by mammalian cells or less optimal furin recognition sequence in current ADAMTS-7 constructs. We tested the idea of co-transfection DNAs of rPro-hCD and furin protease into Expi293 cells to coexpress the two proteins. That yielded overexpression of furin in the media but significantly reduced the production of ADAMTS-7 proteins without increasing the ratio of processed versus unprocessed ADAMTS-7. Next, we focused on the optimization of furin recognition site. We introduced mutation Q216K into rPro-hCD to convert ²¹⁴RQQR²¹⁷ to ²¹⁴RQKR²¹⁷, and named it rPro-hCD-FM2 (SEQ ID No. 2) (FIG. 4 panel A). Q216K mutation significantly increased furin cleavage efficiency of ADAMTS-7 in the same mammalian cell culture compared to the wild type (WT). The ratio of hCD or processed ADAMTS-7 in the raw expression media versus rPro-hCD or unprocessed ADAMTS-7 increased at least 6 fold (FIG. 4 panel B). As a control, a triple mutation R58A/R60A/R217A was introduced into rPro-hCD to silent predicted furin cleavage sites (FIG. 4 panel A). rPro-hCD-3RA completely abolished furin processing and yielded only the unprocessed ADAMTS-7 (FIG. 4 panel B). We conclude that manipulating the sequence of predicted furin recognition site has proved capable of changing the amount of processed aid unprocessed of ADAMTS-7 molecules produced in mammalian cell culture. Thus, both the polypeptide of SEQ ID NO: 01 and that of SEQ ID NO: 02 are working solutions for the problems of protein solubility and expression yield, although the polypeptide of SEQ ID NO: 02 achieves a particularly improved ratio for the furin-processed polypeptide.

### Example B4: Expression Constructs for Production of Recombinant Hybrid ADAMTS-7 Enzymes

The human ADAMTS-7 catalytic domain with the endogenous human prodomain did not yield well folded secreted protein with enzymatic activity. Exchange of the human prodomain with the rat prodomain dramatically increased production of the human catalytic domain from hybrid rat-human constructs. Rat/human ADAMTS-7 chimera sequence (rPro-hCD (Rat 1-217/Human 237-537)-TEV-2Strep-6His, SEQ ID No. 01) encoding rat pro-domain of ADAMTS-7 (amino acids 1-217 of rat sequence UniProt Q1EHB3, which also includes the signal peptide) and catalytic domain of human ADAMTS-7 (amino acids 237-537 of human sequence UniProt Q9UKP4, which also includes a disintegrin domain) followed by TEV cleavage sequence, 2×Strep tag and a His Tag was cloned into the mammalian pcDNA6mycHis (ThermoFischer Scientific) expression vector (or into pcDNA3.4 vector in some embodiments).

Rat/human ADAMTS-7 chimera sequence (rPro-hCD-FM2 (Rat 1-217/Human 237-537 FM2 (Q216K))-TEV-2Strep-6His, SEQ ID No. 02) encoding rat pro-domain of ADAMTS-7 (amino acids 1-217 of rat sequence UniProt Q1EHB3, which also includes the signal peptide) and catalytic domain of human ADAMTS-7 (amino acids 237-537 of human sequence UniProt Q9UKP4, which also includes a disintegrin domain) followed by TEV cleavage sequence, 2×Strep tag and a His Tag was cloned into the mammalian pcDNA6mycHis (ThermoFischer Scientific) expression vector (or into pcDNA3.4 vector in some embodiments). SEQ ID No. 02 contains an additional mutation Glutamine 216 to Lysine within the rat pro-domain sequence (RQQR217↓S to RQKR217↓S), which improved cleavage by Furin for zymogen processing. These expression constructs allow production of recombinant ADAMTS-7 enzyme—either containing at least parts of both domains (e.g., prodomain plus catalytic domain as encoded, in which the prodomain can optionally be preceded by a signal peptide and/or the catalytic domain can optionally be followed by a disintegrin domain) or containing primarily the catalytic domain (e.g., catalytic domain as encoded, for example as generated after furin cleavage between the prodomain and the catalytic domain, which catalytic domain can optionally be followed by a disintegrin domain).

### Example B5 Recombinant Production of Active ADAMTS-7 Enzyme

Expi293 cells (A14635, ThermoFischer Scientific) were grown and transfected in accordance to the manufacturer instruction. In brief, at the final cell density of 2.5×10⁶ cells/mL with >95% viability Expi 293 cells were transfected with 1 mg/liter of vector plasmid DNA as generated according to example 4 using Expifectamine transfection reagent (Thermo Fischer Scientific). Approximately 96 hours post transfection, Expi293 cell culture was centrifuged at 4000 rpm (˜3700 rcf) for 10 mins, and supernatant was collected. Supernatant was neutralized with 50 mM Tris pH8.0, 5 mM CaCl₂), 10 uM ZnCl₂ and 5 mM imidazole pH 8.0 and filtered through 0.22 μm filter.

The filtered supernatant was loaded on Ni-NTA column (GE healthcare #17-3712-06), equilibrated with buffer A (50 mM Tris pH 8.0, 300 mM NaCl, 10% glycerol, 5 mM CaCl₂), 10 μM ZnCl2 and 5 mM Imidazole (pH 8.0)) on Acta FPLC system. The column was washed with 20 volumes of buffer A and the bound proteins were eluted by linear gradient of 20 column volumes up to 100% buffer B (50 mM Tris pH 8.0, 300 mM NaCl, 10% glycerol, 5 mM CaCl₂), 10 μM ZnCl₂ and 500 mM Imidazole (pH 8.0)). The collected fractions were analyzed on the SDS gel and the fractions containing ADAMTS-7 protein were combined and concentrated 10 times. The concentrated material from the Ni-NTA purification was loaded onto superdex S200 (SEC) column equilibrated in column buffer: 50 mM Tris pH 8.0, 300 mM NaCl, 10% glycerol, 5 mM CaCl2) and 10 μM ZnCl2. The collected fractions were analyzed on the SDS gel and the fractions containing ADAMTS-7 protein were combined and concentrated 10 times.

The concentrated material from the Ni-NTA purification was loaded onto superdex S200 (SEC) column equilibrated in column buffer: 50 mM Tris pH 8.0, 300 mM NaCl, 10% glycerol, 5 mM CaCl₂) and 10 μM ZnCl₂. The collected fractions were analyzed on the SDS gel and the fractions containing ADAMTS-7 protein were combined.

Combined S200 fractions were loaded to strep-tactin column (Qiagen #1057981) equilibrated in Buffer A (50 mM Tris 8.0, 300 mM NaCl, 10% glycerol, 5 mM CaCl₂), 10 μM ZnCl₂). The column was washed with 20 column volumes of the buffer A and the bound proteins were eluted by linear gradient of 20 column volumes up to 100% buffer B (50 mM Tris 8.0, 300 mM NaCl, 10% glycerol, 5 mM CaCl₂), 10 uM ZnCl₂ and 2.5 mM D-desthiobiotin). The collected fractions were analyzed on the SDS gel and the fractions containing ADAMTS-7 protein were combined. Combined fractions were dialysed overnight at +4° C. against the storage buffer (20 mM Tris pH 8.0, 300 mM NaCl, 10% glycerol, 5 mM CaCl₂) and 10 μM ZnCl₂).

Dialyzed protein was concentrated to 1 mg/ml, aliquoted, flash frozen in dry ice/ethanol and stored at −80° C. The final yield of purified ADAMTS-7 was 0.5-1 mg per liter of Expi293 cell culture.

In some embodiments, the produced polypeptide is further processed (e.g., by TEV protease) to remove some of the parts C-terminal to the catalytic domain.

### Example B6: Identification of ADAMTS-7 Substrates and Cleavage Assay Development

Based on the interaction mapping data and the apparent fragment size in reducing and non-reducing conditions, the results were consistent with a ADAMTS-7 cleavage site near the second EGF repeat of COMP, however the substrate cleavage site has not been defined.

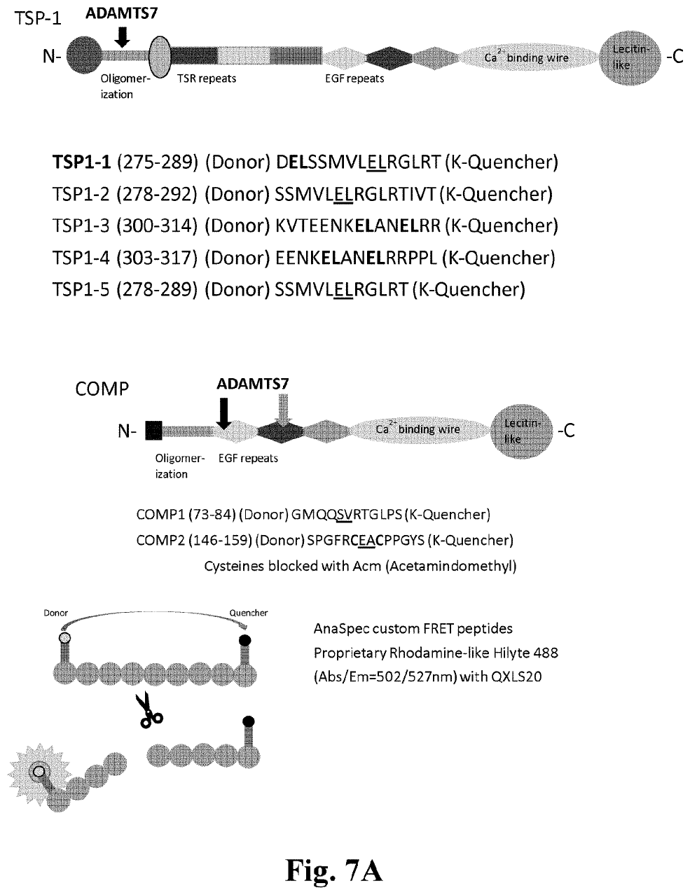

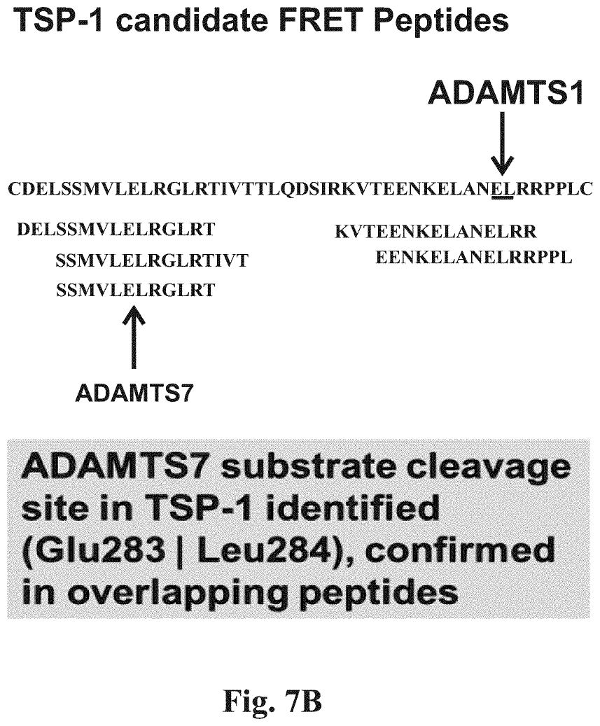

In an attempt to identify ADAMTS-7 substrate cleavage sites, we scanned the potential regions of COMP and TSP1 to identify ADAMTS consensus sites and generated a series of internally-quenched fluorescently-labeled peptides for use with our purified ADAMTS-7 enzyme (FIG. 7). Since ADAMTS-7 was reported to produce the 100 kDA COMP fragment resolvable on a gel under reducing and non-reducing conditions, we hypothesized that cleavage site would not be internal to a disulfide bond. Given the 1-3, 2-4, 5-6 ensemble of disulfide bonds within the EGF repeats, only the E152↓A153 site between the 4th and 5th cysteines committed to separate disulfide bonds was considered, which we attempted to emulate using acetamidomethyl (Acm) modified cysteines in the COMP candidate peptide (See FIG. 7) A second peptide from COMP (amino acids 73-84 GMQQ↓SVRTGLPS) was chosen based on observed ADAMTS cleavage of full-length COMP identified by N-terminal sequencing (data not shown). TSP1 contained a potential E↓LRG upstream from the ADAMTS1 cleavage site at E311↓L312. We chose to analyze this portion of TSP1 using a series of overlapping peptides. The COMP and TSP1 candidate regions contained a modified rhodamine AF488 dye coupled to the N-terminus and a local acceptor QXL520 quencher at the C-terminus that prevents fluorescence in the uncleaned configuration (FIG. 7). Following endopeptidic cleavage, the quencher is released to allow fluorescence signal detection from the substrate's amino terminus. Purified recombinant ADAMTS-7 (as of SEQ ID No. 01 or SEQ ID No. 02) and purified recombinant rADAMTS-7 (rat ADAMTS-7 residues 1-575 with a carboxyl terminal Flag, SEQ ID No. 03) were used to identify substrates from the TSP1 and COMP candidate peptides. ADAMTS-7 was diluted in reaction buffer (20 mM HEPES pH 8.0, 150 mM NaCl, 5 mM CaCl₂), 0.004% Brij, 10 μM ZnCl₂) for a concentration of approximately 100 nM and pre-incubated for 2 hr at room temperature prior to reaction start with 10 μM candidate peptide substrates. There were some differences between optimized buffer data and reported 2 mM Zn²⁺ buffer data (Liu 2006 FASEB J); for example, the published amount of Zinc in assay buffer caused the purified ADAMTS-7 proteins to precipitate. Candidate FRET substrates based on SEQ ID No. 04 to 10 were generated through custom synthesis by Anaspec—AnaSpec, EGT (34801 Campus Drive, Fremont, Calif. 94555, USA); as explained by the manufacturer; AnaSpec, EGT Group's pH insensitive HiLyte™ Fluor dyes are a series of fluorescent labeling dyes with fluorescence emissions that span the full visible and near infrared spectrum. HiLyte™ Fluor dyes and AnaSpec's proprietary quenchers QXL™ have been used as fluorophore and quencher pairs for fluorescence resonance energy transfer (FRET) in our examples) in the reaction buffer. Activity data plotted as Relative Fluorescence Units over time and as a calculated rate (RFU/min) are shown in FIG. 8. Significant activity was not observed with substrates in buffer alone (FIG. 8) or with incubation with purified ADAMTS-7 E389Q catalytic inactive proteins (data not shown).

ADAMTS-7 human catalytic domain constructs rPro-hCD hybrid (SEQ ID No. 01) and optimized furin site construct FM2 (SEQ ID No. 02) demonstrated the greatest specificity for the TSP1 S1 (amino acids 275-289: DELSSMVLELRGLRT, SEQ ID No. C₄). ADAMTS-7 human catalytic domain constructs also cleaved the overlapping TSP1 S2 substrate (amino acids 278-292: SSMVLELRGLRTIVT, SEQ ID No. 05). Substrates 51 and S2 are overlapping at candidate site to E289↓L290 (FIG. 7). Cleavage at this site was confirmed by mass spec using an unlabeled peptide (data not shown). No significant activity was detected at the defined ADAMTS1 cleavage site E311↓La312 from TSP1 S3 (amino acids 300-314: KVTEENKELANERR, SEQ ID No. 06) or TSP1 S4 (amino acids 303-317 EENKELANERRPPL, SEQ ID No. 07). This suggests that the ADAMTS-7 substrate is distinct from ADAMTS1. To confirm the TSP1 E289↓L290 substrate site, a minimum overlap between the 51 and S2 peptides was tested as TSP1 S5 substrate (amino acids 278-289: SSMVLELRGLRT, SEQ ID No. 08). Activity was observed for the S5 substrate with ADAMTS-7 human catalytic domain constructs compared to buffer alone, however the removal of the amino DEL sequence greatly reduced the signal for activity compared to the 51 substrate DELSSMVLELRGLRT, SEQ ID No. 04. No activity was observed with COMP candidate peptides COMP1 amino acids 73-84: GMQQSVRTGLPS, SEQ ID No. 09 or COMP2 amino acids 146-159: SPGFRCEACPPGYS, SEQ ID No. 10.

Activity data from the rat ADAMTS-7 construct identified TSP1 S1 (SEQ ID No. C₄) as the preferred substrate, along with S2 and S5 peptides containing the E289↓L290 cleavage site (FIG. 8). S1 relative fluorescence signal was not as strong for rat ADAMTS-7 compared to the ADAMTS-7 human catalytic domain constructs, resulting in a lower ratio of S1 to S2 activity.

TSP1 S2 substrate (SSMVLELRGLRTIVT, SEQ ID No. 05) presented limited solubility compared to the preferred TSP1 S1 substrate (DELSSMVLELRGLRT, SEQ ID No. 04), potentially due to the additional hydrophobic residues at the carboxyl terminal side (data not shown). To further improve solubility of the 51 peptide, which contained a number of internal hydrophobic residues, modified versions of the 51 peptide ending in -K(QXL520)-NH2 were generated to include an additional hydrophilic moiety: -K(QXL520)-E-NH2 (SEQ ID No. 11), -K(QXL520)-K-NH2 (SEQ ID No. 12) and -K(QXL520)-OH (SEQ ID No. 13). Activity profiles for these substrates were not significantly affected, however the substrate solubility profile was improved with the additional carboxyl glutamic acid (i.e., a glutamic acid that has been conjugated at a carboxyl position on the peptide) added after the QXL520 quencherfor SEQ ID No. 11 (data not shown).

### Example B7 Assay for ADAMTS-7 Enzymatic Activity and Testing of Inhibitory Compounds

Purified recombinant ADAMTS-7 (as of SEQ ID No. 01 or SEQ ID No. 02) was diluted in reaction buffer (20 mM HEPES pH 8.0, 150 mM NaCl, 5 mM CaCl₂, 0.004% Brij, 10 μM ZnCl₂) fora concentration of approximately 20 nM. 25 μl of the solution were transferred into each well of a 384-well white microtiter plate (Greiner Bio-One 781075) and 1 μl test compound solution (modulator/inhibitor dissolved in DMSO, at the corresponding concentration) or pure DMSO as a control were added per well. The enzymatic reaction was initiated by addition of 25 μl of a 1 μM solution of the FRET substrate, HiLyteFluor-488 DELSSMVLELRGLRT-K(QXL520)-E-NH2, (SEQ ID No. 11, custom synthesis by Anaspec) in the reaction buffer. Amino acids DELSSMVLELRGLRT are derived from Thrombospondin-1 sequence (275-289). An additional carboxyl glutamic acid was added after the QXL520 quencher to increase substrate solubility. The microtiter plate was incubated for 120 min at the temperature of 32° C. The increase of fluorescence intensity was measured in appropriate fluorescence plate reader (e.g. TECAN Ultra) using excitation wavelength of 485 nm and emission wavelength of 520 nm. IC50 values were calculated from percentage of inhibition of ADAMTS-7 activity as a function of test compound concentration. IC50 values derived using functional ADAMTS-7 according to SEQ ID No. 01 or SEQ ID No. 02, respectively, were not distinguishable, both laying within the experimental error.

### Example B8: Expression Constructs for Production of Recombinant ADAMTS-12 Enzymes

Rat/human ADAMTS-12 chimera sequence (rPro-hCD (Rat 1-244/Human 241-543)-3×FLAG, SEQ ID 15) encoding rat pro-domain of ADAMTS-12 (amino acids 1-244 of rat sequence UniProt D3ZTJ3, which also includes the signal peptide) and catalytic domain of human ADAMTS-12 (amino acids 241-543 of human sequence UniProt P58397, which also includes a disintegrin domain) followed by 3×FLAG Tag was cloned into the mammalian pcDNA3.4 expression vector.

Rat/human ADAMTS-12 WT demonstrated a better expression profile compared to human ADAMTS-12 (1-543) WT with a human prodomain (FIG. 6). Optimization of the furin cleavage site (L237R) in the context of the human prodomain did not improve the yield of processed CD proteins compared to the rat/human ADAMTS-12 construct. Each ADAMTS-12 construct was mutated in parallel at the catalytic site with an E393Q substitution (EQ) resulting in increased protein yield similar to ADAMTS-7 catalytic mutations. Corresponding yield for the rat/human ADAMTS-12 EQ construct was also higher compared to ADAMTS-12 (1-543) EQ containing the human prodomain.

This expression construct allows production of recombinant ADAMTS-12 enzymes—either containing at least parts of both domains (e.g., prodomain plus catalytic domain as encoded, in which the prodomain can optionally be preceded by a signal peptide and/or the catalytic domain can optionally be followed by a disintegrin domain) or containing primarily the catalytic domain (e.g., catalytic domain as encoded, which can optionally be followed by a disintegrin domain).

### Example B9: Recombinant Production of Active ADAMTS-12 Enzyme

Expi293 cells (Life technologies, A14635) were grown and transfected in accordance to the manufacturer instruction. Briefly, the Expi 293 cells at the final cell density of 2.5×10⁶ cells/mL with >95% viability were transfected by the 1 mg/liter of vector plasmid DNA using Expifectamine transfection reagent (Life technologies, A14525). The overall purification scheme was similar to that used for ratADAMTS-7 (SEQ ID NO: 03).

Approximately 72 hours post transfection, Expi293 cell culture was centrifuged at 4000 rpm (˜3700rcf) for 10 mins. Supernatant was collected and neutralized with 50 mM Tris pH 8.0, 5 mM CaCl₂), 10 μM ZnCl₂ before centrifuged again at 4000 rpm (˜3700rcf) for 10 min. Final supernatant was filtered through 0.22 μm filter.

The filtered supernatant was incubated overnight at 4° C. with anti-FLAG M2 affinity gel (Sigma-Aldrich A2220) equilibrated with bufferA (50 mM Tris pH 8.0, 300 mM NaCl, 10% glycerol, 5 mM CaCl₂), 10 μM ZnCl₂). The gel was collected and washed with 10 bed volumes of buffer A. The bound proteins were eluted by 100% buffer B (50 mM Tris pH 8.0, 300 mM NaCl, 10% glycerol, 5 mM CaCl₂), 10 μM ZnCl₂, 150 ng/μl FLAG peptide (Sigma-Aldrich F4799)). The collected fractions were analyzed on the SDS gel. The fractions containing ADAMTS-12 protein were combined and concentrated 10 times.

The concentrated material from the FLAG affinity purification was loaded onto superdex S200 (SEC) column equilibrated in column buffer 20 mM Tris pH 8.0, 300 mM NaCl, 10% glycerol, 5 mM CaCl₂) and 10 μM ZnCl₂. The collected fractions were analyzed on the SDS gel. Fractions containing ADAMTS-12 protein were combined and concentrated to 0.5 mg/ml. Aliquoted proteins were flash frozen in liquid nitrogen and stored at −80° C.

### Example B10: Assay for ADAMTS-12 Enzymatic Activity and Testing of Inhibitory Compounds

Purified recombinant ADAMTS-12 was diluted in the reaction buffer (20 mM HEPES pH 8.0; 10 mM NaCl; 7.5 mM CaCl₂); 0,004% Brij; 7.5 μM ZnCl₂; 0.1% SmartBlock (Candor Bioscience 113125)) to the concentration of approximately 20 nM and 25 μl were transferred into each single well of 384-well white microtiter plate (Greiner Bio One 781075). 1 μl of the inhibitor compound solution (dissolved in DMSO, at the corresponding concentration) or pure DMSO as a control was added to the same wells. The enzymatic reaction was initiated by addition of 25 μl of 2 μM solution of the FRET substrate HiLyte Fluor 488-DELSSMVLELRGLRT-K(QXL520)E-NH2; (cf. SEQ ID No. 11) in the reaction buffer. It was surprisingly found that the same substrate could be used for the paralogs ADAMTS-7 and ADAMTS-12. The microtiter plate was incubated for 120 min at the temperature of 32° C. The increase of fluorescence intensity was measured in appropriate fluorescence plate reader (e.g. TECAN Ultra) using excitation wavelength of 485 nm and emission wavelength of 520 nm. IC50 values were calculated from percentage of inhibition of ADAMTS-12 activity as a function of test compound concentration.

### Example B11: Selectivity Assay for ADAMTS-7 and/or ADAMTS-12 Modulators

The respective enzyme (see table 2 below) was diluted in reaction buffer (50 mM Tris, 2.5 μM ZnCl₂, 0.05% BSA, 0.001% Brij, pH 7.5 for ADAM17; 50 mM Tris 7.5, 150 mM NaCl, 10 mM CaCl₂, 0.05% Brij for all other enzymes) to the respective concentration and 25 μl were transferred into each well of a 384-well white microtiter plate (Greiner Bio One 781075). 1 μl test compound solution (dissolved in DMSO, at the corresponding concentration) or pure DMSO as a control was added per well. The enzymatic reaction was initiated by addition of 25 μl of the respective concentration of the respective FRET substrate (see table 2 below) in reaction buffer. The microtiter plate was incubated for 120 min at the temperature of 32° C. The increase of fluorescence intensity was measured in appropriate fluorescence plate reader (e.g. TECAN Ultra) using the respective wavelengths for excitation and emission. IC50 values were calculated from percentage of inhibition of enzyme activity as a function of test compound concentration.

TABLE 2
Assay conditions for the evaluation of the selectivity
of ADAMTS-7 modulators.

| | | Conc. | | | Conc. | Source | Excitation | Emission |
|---|---|---|---|---|---|---|---|---|
| Enzyme | Source Enzyme | Enzyme | | FRET substrate | Substrate | Substrate | Wavelength | Wavelength |
| ADAMTS4 | R&D 4307-AD | 50 | nM | Dabcyl-EEVKAKVQPY- | 1 μM | Jerini Peptide | 340 nm | 480 nm |
| | | | | Glu(Edans)-NH2 | | Technologies | | |
| | | | | (cf. SEQ ID | | | | |
| | | | | No. 14) | | | | |
| ADAMTS5 | R&D2198-AD | 100 | nM | Dabcyl-EEVKAKVQPY- | 25 μM | Jerini Peptide | 340 nm | 480 nm |
| | | | | Glu(Edans)-NH2 | | Technologies | | |
| | | | | (cf. SEQ ID | | | | |
| | | | | No. 14) | | | | |
| MMP12 | R&D 917-MPB, | 1 | nM | Mca-PLGLEEA- | 10 μM | BachemM- | 325 nm | 393 nm |
| | activated | | | Dap(Dnp)-NH2 | | 2670 | | |
| | according to | | | (cf. SEQ ID | | | | |
| | manufacturers | | | No. 17) | | | | |
| | instruction | | | | | | | |
| MMP15 | R&D 916-MP, | 6 | nM | Mca-KPLGL- | 20 μM | R&D ES010 | 320 nm | 405 nm |
| | activated | | | Dpa-AR-NH2 (cf. | | | | |
| | according to | | | SEQ ID No. 18) | | | | |
| | manufacturers | | | (Neumann, U. | | | | |
| | instruction | | | etal., 2004, Anal. | | | | |
| | | | | Biochem. 328: | | | | |
| | | | | 166-173) | | | | |
| MMP2 | R&D 902-MP, | 0.06 | nM | Mca-PLGL-Dpa- | 20 μM | R&D ES001 | 320 nm | 405 nm |
| | activated | | | AR-NH2 | | | | |
| | according to | | | (cf. SEQ ID | | | | |
| | manufacturers | | | No. 19) | | | | |
| | instruction | | | | | | | |
| ADAM17 | R&D 930-ADB | 20 | nM | Mca-PLAQAV- | 20 μM | BachemM- | 320 nm | 405 nm |
| | | | | Dap(Dnp)- | | 2255 | | |
| | | | | RSSSR-NH2 | | | | |
| | | | | (cf. SEQ ID | | | | |
| | | | | No. 20) | | | | |

Abbreviations: Dabcyl: e.g. Dabcyl quencher in 3-DAB form N-[4-(4-dimethylamino)phenylazo]benzoic acid; Glu(Edans): e.g. EDANS fluor (5-((2-Aminoethyl)amino)naphthalene-1-sulfonic acid) at modified Glutamic acid; Mca: (7-Methoxycoumarin-4-yl)acetyl; Dap(Dnp): e.g. N-beta-(2,4-dinitrophenyl)-L-2,3-diaminopropionic acid; Dpa: N-3-(2,4-Dinitrophenyl)-L-2,3-diaminopropionyl.

TABLE 3

| Example Nr. | ADAMTS7-IC₅₀ [nM] | ADAMTS4-IC₅₀ [nM] |
|---|---|---|
| 1 | 13 | 580 |
| 2 | 210 | 50000 |
| 3 | 79 | 2200 |
| 4 | 260 | 6600 |
| 5 | 120 | 4600 |
| 6 | 620 | 50000 |
| 7 | 62 | 8500 |
| 8 | 7 | 175 |
| 9 | 26 | 480 |
| 10 | 21 | 450 |
| 11 | 470 | 10000 |
| 12 | 980 | 12000 |
| 13 | 47 | 950 |
| 14 | 400 | 17000 |
| 15 | 140 | 4750 |
| 16 | 355 | 24000 |
| 17 | 32 | 600 |
| 18 | 170 | 2200 |
| 19 | 170 | 22000 |
| 20 | 26 | 4800 |
| 21 | 200 | 2800 |
| 22 | 10 | 3000 |
| 23 | 30 | 4500 |
| 24 | 140 | 2900 |
| 25 | 550 | 50000 |
| 26 | 480 | 50000 |
| 27 | 100 | 6000 |
| 28 | 870 | 67000 |
| 29 | 5 | 250 |
| 30 | 12 | 500 |
| 31 | 41 | 4250 |
| 32 | 35 | 910 |
| 33 | 58 | 1400 |
| 34 | 26 | 1420 |
| 35 | 74 | 3100 |
| 36 | 110 | 5500 |
| 37 | 180 | 7600 |
| 38 | 10 | 2800 |
| 39 | 710 | 50000 |
| 40 | 980 | 25000 |
| 41 | 55 | 1700 |
| 42 | 350 | 12000 |
| 43 | 320 | 13000 |
| 44 | 250 | 5000 |
| 45 | 69 | 1450 |
| 46 | 52 | 4690 |
| 47 | 98 | 7650 |
| 48 | 265 | 14500 |
| 49 | 70 | 15000 |
| 50 | 191 | 50000 |
| 51 | 89 | 15000 |
| 52 | 55 | 14000 |
| 53 | 145 | 50000 |
| 54 | 670 | 25000 |
| 55 | 240 | 19000 |
| 56 | 129 | 1800 |
| 57 | 190 | 17000 |
| 58 | 550 | 5600 |
| 59 | 550 | 11300 |
| 60 | 259 | 8400 |
| 61 | 128 | 9950 |
| 62 | 42 | 16000 |
| 63 | 69 | 6000 |
| 64 | 240 | 50000 |
| 65 | 170 | 19000 |
| 66 | 380 | 50000 |
| 67 | 260 | 9300 |
| 68 | 120 | 8300 |
| 69 | 98 | 35000 |
| 70 | 76 | 10000 |
| 71 | 47 | 13100 |
| 72 | 130 | 6200 |
| 73 | 60 | 5100 |
| 74 | 33 | 5900 |
| 75 | 14 | 4100 |
| 76 | 8 | 2770 |
| 77 | 24 | 4380 |
| 78 | 191 | 19000 |
| 79 | 78 | 11000 |
| 80 | 250 | 2900 |
| 81 | 37 | 1500 |
| 82 | 48 | 3100 |
| 83 | 120 | 11000 |
| 84 | 150 | 4000 |
| 85 | 250 | 6500 |
| 86 | 63 | 8500 |
| 87 | 98 | 20000 |
| 88 | 98 | 13000 |
| 89 | 6 | 975 |
| 90 | 16 | 3100 |
| 91 | 18 | 1800 |
| 92 | 78 | 4000 |
| 93 | 8 | 2200 |
| 94 | 6 | 890 |
| 95 | 10 | 980 |
| 96 | 48 | 2200 |
| 97 | 10 | 1900 |
| 98 | 25 | 1100 |
| 99 | 52 | 6000 |
| 100 | 8 | 680 |
| 101 | 7 | 14000 |
| 102 | 62 | 2900 |
| 103 | 2 | 200 |
| 104 | 10 | 2200 |
| 105 | 190 | 11000 |
| 106 | 190 | >50000 |
| 107 | 7 | 310 |
| 108 | 12 | 600 |
| 109 | 19 | 1100 |
| 110 | 5 | 400 |
| 111 | 35 | 1200 |
| 112 | 14 | 780 |
| 113 | 39 | 2400 |
| 114 | 15 | 1400 |
| 115 | 87 | 6600 |
| 116 | 78 | 5500 |
| 117 | 20 | 3300 |
| 118 | 46 | 4500 |
| 119 | 13 | 2300 |
| 120 | 170 | 6000 |
| 121 | 78 | 2600 |
| 122 | 11 | 2500 |
| 123 | 7 | 910 |
| 124 | 13 | 2800 |
| 125 | 7 | 1000 |
| 126 | 190 | 12000 |
| 127 | 10 | 2400 |
| 128 | 29 | 2400 |
| 129 | 18 | 1700 |
| 130 | 14 | 2800 |
| 131 | 37 | 2500 |
| 132 | 230 | 7400 |
| 133 | 50 | 3200 |
| 134 | 190 | 6200 |
| 135 | 48 | 6600 |
| 136 | 27 | 3500 |
| 137 | 37 | 1000 |
| 138 | 590 | 50000 |
| 139 | 69 | >50000 |
| 140 | 4 | 360 |
| 141 | 4 | 320 |
| 142 | 4 | 320 |
| 143 | 5 | 200 |
| 144 | 2 | 130 |
| 145 | 2 | 41 |
| 146 | 3 | 108 |
| 147 | 3 | 46 |
| 148 | 12 | 2500 |
| 150 | 38 | 2000 |
| 151 | 320 | 17000 |
| 152 | 560 | 5000 |
| 153 | 83 | 8900 |
| 154 | 190 | 7900 |
| 155 | 220 | >50000 |
| 156 | 59 | 15000 |
| 157 | 8 | 460 |
| 158 | 100 | 2800 |
| 159 | 74 | 3700 |
| 160 | 60 | 1000 |
| 161 | 130 | 7100 |
| 162 | 200 | 13000 |

### Example B12 Rat Carotid Artery Balloon Injury Model

The balloon injury model is an important method to study the molecular and cellular mechanisms involved in vascular smooth muscle dedifferentiation, neointima formation and vascular remodeling.

The left carotid is injured using a balloon catheter with the right carotid serving as a negative control; the inflated balloon denudes the endothelium and distends the vessel wall. Following injury, potential therapeutic strategies such as the use of pharmacological compounds can be evaluated.

Following sedation to a surgical plane, male Sprague-Dawley rats are laid supine on a sterile surface, the neck area is shaved, cleansed and the surgical area aseptically draped to prevent contamination at the surgical site. A straight incision is made centrally in the neck region from below the chin to the top of the sternum. With continued blunt dissection, the left common carotid artery and the distal aspect of the carotid artery cephalic to the internal and external bifurcation are isolated and made free of overlying fascia and adjacent nerves. Sterile sutures and an arterial clamp are used to control blood flow in order to perform the injury. Isolating a section on the internal carotid artery and using sterile small microscissors, a transverse arteriotomy on the branch is performed. The uninflated 2 French arterial balloon catheter (Edwards Life Sciences, Germany) is inserted through the arteriotomy, inflated and moved down the entire length of the common carotid artery to the aortic arch. The balloon is inflated to a predetermined volume, deflated and withdrawn through the arteriotomy. Following adequate hemostasis, all remaining sutures are removed and the operational field is closed up. Neointimal thickening representing proliferative vascular smooth muscle cells usually peaks at 2 weeks after injury. Vessels are harvested at this time point for histological assessment of neointimal development, vascular growth, molecular analysis as well as gene and protein expression.

### CLAUSES

The following clauses refer to further embodiments disclosed herein:
1. A compound of general formula (I):

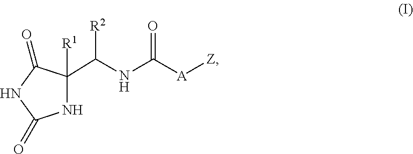

in which
R¹ represents a group selected from hydrogen, (C₁-C₆)-alkyl, (C₃-C₆)-cycloalkyl, 5- to 6-membered heterocycloalkyl, 5- to 6-membered heteroaryl and phenyl,
   wherein said (C₁-C₆)-alkyl, (C₃-C₆)-cycloalkyl, 5- to 6-membered heterocycloalkyl, 5- to 6-membered heteroaryl and phenyl are optionally substituted, with one or two groups independently selected from cyano, halogen, amino, hydroxy, oxo, (C₁-C₃)-alkyl, (C₁-C₄)-alkoxy, (C₁-C₄)-alkylcarbonyl, mono-(C₁-C₄)-alkylamino, di-(C₁-C₄)-alkylamino, (C₁-C₄)-alkylsulfonyl, (C₃-C₆)-cycloalkyl,
   wherein each said (C₁-C₃)-alkyl, (C₃-C₆)-cycloalkyl and, (C₁-C₄)-alkoxy is optionally substituted with up to 5 fluorine atoms
R² represents a group selected from hydrogen or (C₁-C₄)-alkyl,
   wherein said (C₁-C₄)-alkyl is optionally substituted with up to five fluorine atoms,
A represents a group selected from 5-membered heteroaryl,
   wherein said 5-membered heteroaryl is optionally substituted with one, two or three groups independently selected from halogen, cyano, hydroxy, methylsulfonyl, (C₃-C₆)-cycloalkyl, (C₁-C₄)-alkyl, and (C₁-C₄)-alkoxy,
   wherein each said (C₃-C₆)-cycloalkyl, (C₁-C₄)-alkylor (C₁-C₄)-alkoxy is optionally substituted with up to three fluorine atoms,
Z represents a group selected from 6- to 10-membered aryl and 5- to 10-membered heteroaryl,
   wherein said 6- to 10-membered aryl or 5- to 10-membered heteroaryl is optionally substituted with one, two or three groups independently selected from halogen, cyano, hydroxy, (C₁-C₄)-alkylsulfonyl, (C₃-C₆)-cycloalkyl, (C₁-C₄)-alkyl, and (C₁-C₄)-alkoxy,
      wherein each said (C₁-C₄)-alkyl and (C₁-C₄)-alkoxy is optionally substituted with (C₃-C₆)-cycloalkyl and optionally substituted with up to five fluorine atoms, and pharmaceutically acceptable salts thereof, solvates thereof and the solvates of the salts thereof.
2. The compound of clause 1, with the proviso that the following compounds (Xa) to (Xi) are excluded
4-(4-chlorophenyl)-N-[[4-(1-methylimidazol-2-yl)-2,5-dioxo-imidazolidin-4-yl]methyl]-1H-pyrrole-2-carboxamide

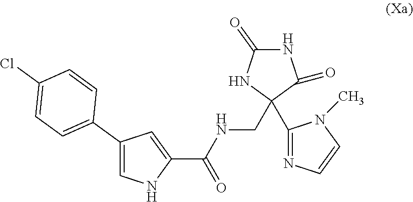 [SMILES: Cn1ccnc1C1(CNC(=O)c2cc(-c3ccc(Cl)cc3)c[nH]2)NC(=O)NC1=O]

1-(4-fluorophenyl)-N-[[4-(1-methylimidazol-2-yl)-2,5-dioxo-imidazolidin-4-yl]methyl]pyrazole-4-carboxamide

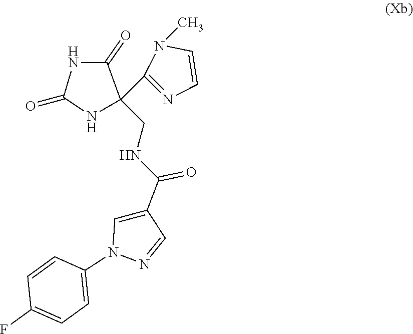 [SMILES: Cn1ccnc1C1(CNC(=O)c2cnn(-c3ccc(F)cc3)c2)NC(=O)NC1=O]

1-methyl-N-[[4-(1-methylimidazol-2-yl)-2,5-dioxo-imidazolidin-4-yl]methyl]-5-phenyl-pyrrole-2-carboxamide

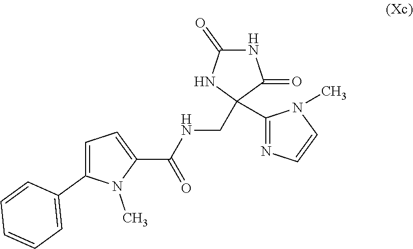 [SMILES: Cn1ccnc1C1(CNC(=O)c2ccc(-c3ccccc3)n2C)NC(=O)NC1=O]

4-(5-chloro-2-thienyl)-N-[[4-(1-methylimidazol-2-yl)-2,5-dioxo-imidazolidin-4-yl]methyl]-1H-pyrrole-3-carboxamide

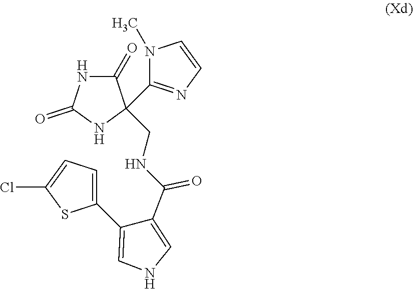 [SMILES: Cn1ccnc1C1(CNC(=O)c2c[nH]cc2-c2ccc(Cl)s2)NC(=O)NC1=O]

4-methyl-N-[[4-(1-methylimidazol-2-yl)-2,5-dioxo-imidazolidin-4-yl]methyl]-2-(2-thienyl)thiazole-5-carboxamide

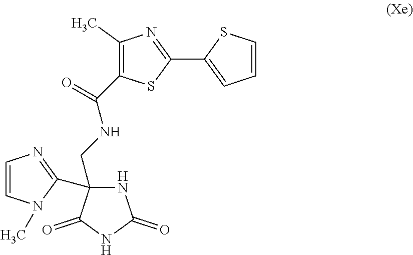 [SMILES: Cc1nc(-c2cccs2)sc1C(=O)NCC1(c2nccn2C)NC(=O)NC1=O]

N-[[4-(1-methylimidazol-2-yl)-2,5-dioxo-imidazolidin-4-yl]methyl]-2-(4-pyridyl)thiazole-5-carboxamide

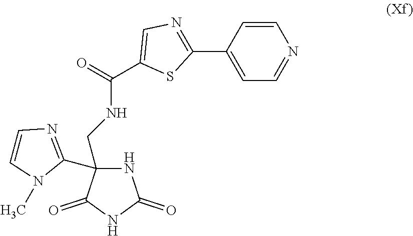 [SMILES: Cn1ccnc1C1(CNC(=O)c2cnc(-c3ccncc3)s2)NC(=O)NC1=O]

5-methyl-N-[[4-(1-methylimidazol-2-yl)-2,5-dioxo-imidazolidin-4-yl]methyl]-1-(4-pyridyl)pyrazole-4-carboxamide

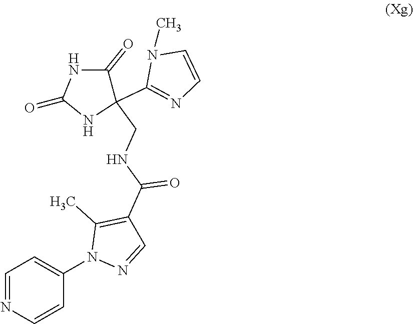 [SMILES: Cc1c(C(=O)NCC2(c3nccn3C)NC(=O)NC2=O)cnn1-c1ccncc1]

N-[[4-(1-methylimidazol-2-yl)-2,5-dioxo-imidazolidin-4-yl]methyl]-5-(1H-pyrazol-3-yl)thiophene-2-carboxamide

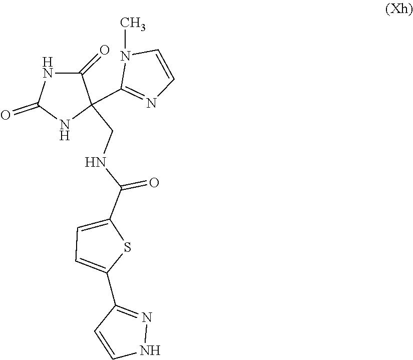 [SMILES: Cn1ccnc1C1(CNC(=O)c2ccc(-c3cc[nH]n3)s2)NC(=O)NC1=O]

N-[[4-(1-methylimidazol-2-yl)-2,5-dioxo-imidazolidin-4-yl]methyl]-5-phenyl-1H-pyrazole-3-carboxamide

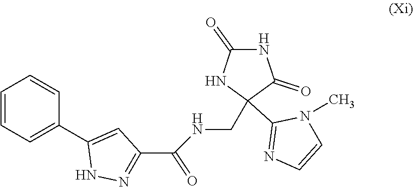 [SMILES: Cn1ccnc1C1(CNC(=O)c2cc(-c3ccccc3)[nH]n2)NC(=O)NC1=O]

3. A compound of general formula (I) according to Clause 1 or Clause 2,
   in which
   R¹ represents a group selected from hydrogen, (C₁-C₆)-alkyl, (C₃-C₆)-cycloalkyl, 5- to 6-membered heterocycloalykl, 5- to 6-membered heteroaryl and phenyl
      wherein said (C₁-C₆)-alkyl (C₃-C₆)-cycloalkyl, 5- to 6-membered heterocycloalkyl, 5- to 6-membered heteroaryl and phenyl are optionally substituted, with one or two groups independently selected from cyano, halogen, amino, hydroxy, oxo, C₁-C₃-alkyl, (C₁-C₄)-alkoxy, (C₁-C₄)-alkylcarbonyl, mono-(C₁-C₄)-alkylamino, di-(C₁-C₄)-alkylamino, (C₁-C₄)-alkylsulfonyl, (C₃-C₆)-cycloalkyl,
      wherein each said (C₁-C₃)-alkyl, (C₃-C₆)-cycloalkyl, and (C₁-C₄)-alkoxy is optionally substituted with up to 5 fluorine atoms,
   R² represents a group selected from hydrogen, (C₁-C₄)-alkyl,
      wherein said (C₁-C₄)-alkyl is optionally substituted with up to five halogen atoms,
   A represents a group selected from 5-membered aza-heteroaryl,
      wherein said 5-membered aza-heteroaryl is optionally substituted with one, two or three groups independently selected from halogen, cyano, hydroxy, methylsulfonyl, (C₃-C₆)-cycloalkyl, (C₁-C₄)-alkyl, and (C₁-C₄)-alkoxy,
      wherein each said (C₁-C₄)-alkyl, (C₁-C₄)-alkoxy is optionally substituted with up to three fluorine atoms,
   Z represents a group selected from Phenyl and 5- to 6-membered heteroaryl,
      optionally substituted, with one, two or three groups independently selected from halogen, cyano, hydroxy, (C₁-C₄)-alkylsulfonyl, (C₃-C₆)-cycloalkyl, (C₁-C₄)-alkyl, and (C₁-C₄)-alkoxy,
         wherein each said (C₁-C₄)-alkyl and (C₁-C₄)-alkoxy is optionally substituted with (C₃-C₆)-cycloalkyl and optionally substituted with up to five fluorine atoms,
   and pharmaceutically acceptable salts thereof, solvates thereof and the solvates of the salts thereof.
4. A compound of general formula (I) according to any one of Clauses 1-3, in which
   R¹ represents a group selected from hydrogen, (C₁-C₆)-alkyl, (C₃-C₆)-cycloalkyl, 5- to 6-membered heterocycloalkyl, 5- to 6-membered heteroaryl and phenyl
      wherein said (C₁-C₆)-alkyl (C₃-C₆)-cycloalkyl, 5- to 6-membered heterocycloalkyl, 5- to 6-membered heteroaryl and phenyl are optionally substituted, with one or two groups independently selected from cyano, halogen, amino, hydroxy, oxo, C₁-C₃-alkyl, (C₁-C₄)-alkoxy, (C₁-C₄)-alkylcarbonyl, mono-(C₁-C₄)-alkylamino, di-(C₁-C₄)-alkylamino, (C₁-C₄)-alkylsulfonyl, (C₃-C₆)-cycloalkyl,
      wherein each said C₁-C₃-alkyl, (C₃-C₆)-cycloalkyl and (C₁-C₄)-alkoxy is optionally substituted with up to 5 fluorine atoms
   R² represents a group selected from hydrogen or methyl
   A represents a group selected from triazolyl, oxazolyl, isoxazolyl, pyrazolyl, thiazolyl, 1,2,4-oxadiazolyl and 1,3,4-oxadiazolyl
      optionally substituted with one, two or three groups independently selected from halogen, cyano, hydroxy, methylsulfonyl, (C₃-C₆)-cycloalkyl, (C₁-C₄)-alkyl and (C₁-C₄)-alkoxy
      wherein each said (C₁-C₄)-alkyl, (C₁-C₄)-alkoxy is optionally substituted with up to three fluorine atoms,
   Z represents a group selected from Phenyl and 5- to 6-membered heteroaryl,
      optionally substituted, with one, two or three groups independently selected from halogen, cyano, hydroxy, methylsulfonyl, (C₃-C₆)-cycloalkyl, (C₁-C₄)-alkyl, (C₁-C₄)-alkoxy,
         wherein each said (C₁-C₄)-alkyl and (C₁-C₄)-alkoxy is optionally substituted with, (C₃-C₆)-cycloalkyl and optionally substituted with up to five fluorine atoms,
   and pharmaceutically acceptable salts thereof, solvates thereof and the solvates of the salts thereof.
5. Compound of the formula (I) according to Clause 1, 2 3, or 4, in which
   R¹ represents a group selected from hydrogen, (C₁-C₆)-alkyl, (C₃-C₆)-cycloalkyl, 5- to 6-membered heterocycloalkyl, 5- to 6-membered heteroaryl and phenyl
      wherein said (C₁-C₆)-alkyl (C₃-C₆)-cycloalkyl, 5- to 6-membered heterocycloalkyl, 5- to 6-membered heteroaryl and phenyl are optionally substituted, with one or two groups independently selected from cyano, halogen, amino, hydroxy, oxo, C₁-C₃-alkyl, (C₁-C₄)-alkoxy, (C₁-C₄)-alkylcarbonyl, mono-(C₁-C₄)-alkylamino, di-(C₁-C₄)-alkylamino, (C₁-C₄)-alkylsulfonyl, (C₃-C₆)-cycloalkyl,
      wherein each said C₁-C₃-alkyl, (C₃-C₆)-cycloalkyl, (C₁-C₄)-alkoxy is optionally substituted with up to 5 fluorine atoms
   R² represents a group selected from hydrogen or methyl,
   A represents a triazolyl
      optionally substituted with one, two or three groups independently selected from halogen, cyano, hydroxy, methylsulfonyl, (C₃-C₆)-cycloalkyl, (C₁-C₄)-alkyl, and (C₁-C₄)-alkoxy,
      wherein each said (C₁-C₄)-alkyl, (C₁-C₄)-alkoxy is optionally substituted with up to three fluorine atoms,
   Z represents a group selected from Phenyl and 5 to 6 membered heteroaryl,
      optionally substituted, with one, two or three groups independently selected from halogen, cyano, hydroxy, methylsulfonyl, (C₃-C₆)-cycloalkyl, (C₁-C₄)-alkyl, and (C₁-C₄)-alkoxy,
         wherein each said (C₁-C₄)-alkyl and (C₁-C₄)-alkoxy is optionally substituted with (C₃-C₆)-cycloalkyl and optionally substituted with up to five fluorine atoms,
   and pharmaceutically acceptable salts thereof, solvates thereof and the solvates of the salts thereof.
6. Compound of the formula (I) according to Clause 1 to 5 selected from the group consisting of
N-{[(4R)-4-cyclopropyl-2,5-dioxoimidazolidin-4-yl]methyl}-2-(2,4-difluorophenyl)-2H-1,2,3-triazole-4-carboxamide

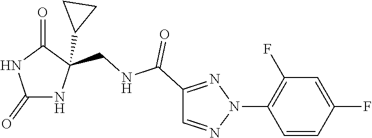 [SMILES: O=C1NC(=O)[C@](CNC(=O)c2cnn(-c3ccc(F)cc3F)n2)(C2CC2)N1]

N-{[(4R)-4-cyclopropyl-2,5-dioxoimidazolidin-4-yl]methyl}-2-(4-fluorophenyl)-2H-1,2,3-triazole-4-carboxamide

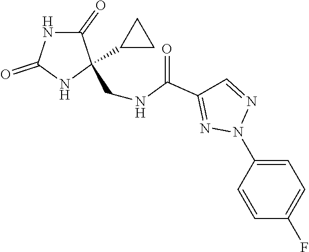 [SMILES: O=C1NC(=O)[C@](CNC(=O)c2cnn(-c3ccc(F)cc3)n2)(C2CC2)N1]

2-(3-chloro-4-fluorophenyl)-N-{[(4R)-4-cyclopropyl-2,5-dioxoimidazolidin-4-yl]methyl}2H-1,2,3-triazole-4-carboxamide

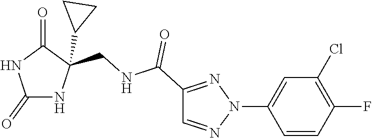 [SMILES: O=C1NC(=O)[C@](CNC(=O)c2cnn(-c3ccc(F)c(Cl)c3)n2)(C2CC2)N1]

ent-N-[(2,5-dioxo-4-(propan-2-yl)imidazolidin-4-yl)methyl]-2-(4-fluorophenyl)-2H-1,2,3-triazole-4-carboxamide

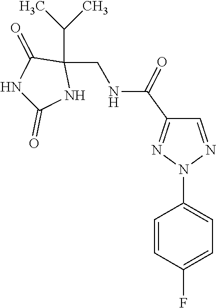 [SMILES: CC(C)C1(CNC(=O)c2cnn(-c3ccc(F)cc3)n2)NC(=O)NC1=O]

ent-N-[(4-cyclobutyl-2,5-dioxoimidazolidin-4-yl)methyl]-2-(4-fluorophenyl)-2H-1,2,3-triazole-4-carboxamide

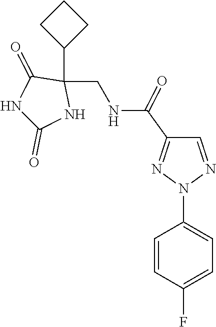 [SMILES: O=C1NC(=O)C(CNC(=O)c2cnn(-c3ccc(F)cc3)n2)(C2CCC2)N1]

ent-N-[(4-cyclobutyl-2,5-dioxoimidazolidin-4-yl)methyl]-2-(3,4,5-trifluorophenyl)-2H-1,2,3-triazole-4-carboxamide

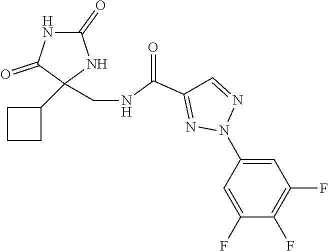 [SMILES: O=C1NC(=O)C(CNC(=O)c2cnn(-c3cc(F)c(F)c(F)c3)n2)(C2CCC2)N1]

ent-N-[(4-cyclobutyl-2,5-dioxoimidazolidin-4-yl)methyl]-2-(3-fluorophenyl)-2H-1,2,3-triazole-4-carboxamide

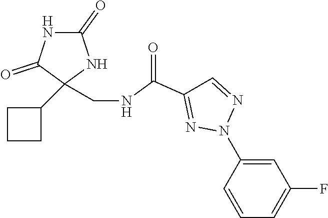 [SMILES: O=C1NC(=O)C(CNC(=O)c2cnn(-c3cccc(F)c3)n2)(C2CCC2)N1]

and
ent-N-[(4-cyclobutyl-2,5-dioxoimidazolidin-4-yl)methyl]-2-(3,4-difluorophenyl)-2H-1,2,3-triazole-4-carboxamide

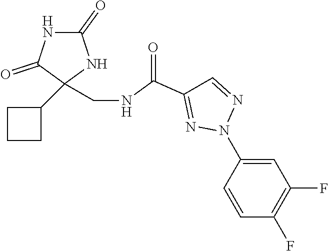 [SMILES: O=C1NC(=O)C(CNC(=O)c2cnn(-c3ccc(F)c(F)c3)n2)(C2CCC2)N1]

and pharmaceutically acceptable salts thereof, solvates thereof and the solvates of the salts thereof.
7. Compound of formula (I) for the treatment and/or prevention of diseases.
8. Compound of formula (I) for use in a method for the treatment and/or prevention of heat diseases, vascular diseases, and/or cardiovascular diseases, lung diseases, inflammatory diseases, fibrotic diseases, metabolic diseases, cardiometabolic diseases.
9. Compound of formula (I) for use in a method for the treatment and/or prevention of atherosclerosis, athersclerosis-related diseases such as coronary artery disease or peripheral vascular disease/arterial occlusive disease as well as post-surgery complications of these diseases such as restenosis after angioplasty.
10. Use of a compound according to formula (I) for the manufacture of a pharmaceutical composition for the treatment and/or prevention of atherosclerosis, athersclerosis-related diseases such as coronary artery disease or peripheral vascular disease/arterial occlusive disease as well as post-surgery complications of these diseases such as restenosis after angioplasty.
11. Use of a compound as defined in any of Clauses 1 to 6 for the manufacture of a pharmaceutical composition for the treatment and/or prevention of atherosclerosis, athersclerosis-related diseases such as coronary artery disease or peripheral vascular disease/arterial occlusive disease as well as post-surgery complications of these diseases such as restenosis after angioplasty.
12. Pharmaceutical composition comprising a compound as defined in any of Clauses 1 to 6 and one or more pharmaceutically acceptable excipients.
13. Pharmaceutical composition of clause 12 comprising one or more first active ingredients, in particular compounds of general formula (I) according to any one of clauses 1 to 6, and one or more further active ingredients, in particular one or more additional therapeutic agents selected from the group consisting of angiotensin-converting enzyme inhibitors, angiotensin-receptor blockers, mineralocorticoid-receptor antagonists, endothelin antagonists, renin inhibitors, calcium blockers, beta-receptor blockers, vasopeptidase inhibitors, Sodium-Glucose-Transport-Antagonists, Metformin, Pioglitazones and Dipeptidyl-peptidase-IV inhibitors.
14. The pharmaceutical composition as defined in Clause 12 or 13 for the treatment and/or prevention of atherosclerosis, athersclerosis-related diseases such as coronary artery disease or peripheral vascular disease/arterial occlusive disease as well as post-surgery complications of these diseases such as restenosis after angioplasty.
15. Method for the treatment and/or prevention atherosclerosis, athersclerosis-related diseases such as coronary artery disease or peripheral vascular disease/arterial occlusive disease as well as post-surgery complications of these diseases such as restenosis after angioplastyin a human or other mammal, comprising administering to a human or other mammal in need thereof a therapeutically effective amount of one or more compounds as defined in any of Clauses 1 to 6, or of a pharmaceutical composition as defined in any of clauses 12-13.
16. A recombinant nucleic acid for expression of an ADAMTS-7 polypeptide that comprises a rodent prodomain of ADAMTS-7 as a first portion and a functional human ADAMTS-7 as a second portion
17. A recombinant nucleic acid for expression of an ADAMTS-7 polypeptide, wherein the polypeptide comprises
   a first portion having a sequence identity of >80% with the sequence of residues 1-217 of SEQ ID NO: 1 or with the sequence of residues 1-217 of SEQ ID NO: 2; and
   a second portion having a sequence identity of >80% with the sequence of residues 218-518 of SEQ ID NO: 1.
18. The recombinant nucleic acid of any one of clauses 16 or 17, wherein the first portion of the polypeptide comprises residues 1-217 of SEQ ID NO: 1 or residues 1-217 of SEQ ID NO: 2, and/or wherein the second portion amino acid sequence comprises residues 218-518 of SEQ ID NO: 1.
19. A recombinant nucleic acid for expression of an ADAMTS-7 polypeptide, wherein the recombinant nucleic acid encodes for a recombinant polypeptide that comprises
   a first portion having an amino acid sequence that aligns with a segment of an ADAMTS-7 prodomain amino acid sequence from a first species with a Needleman-Wunsch score greater than 700 when BLOSUM62 matrix, a gap opening penalty of 11, and a gap extension penalty of 1 are used; and
   a second portion having an amino acid sequence that aligns with a segment of an ADAMTS-7 catalytic domain amino acid sequence from a second species with a Needleman-Wunsch score greater than 1000 when BLOSUM62 matrix, a gap opening penalty of 11, and a gap extension penalty of 1 are used, wherein optionally the first species is rat and the second species is human.
20. The recombinant nucleic acid of any one of clauses 16 to 19, wherein the motif RQQR within the first portion of the polypeptide is altered, preferably into RQKR.
21. A recombinant nucleic acid for expression of an ADAMTS-12 polypeptide that comprises a rodent prodomain of ADAMTS-12 as a first portion and a functional human ADAMTS-12 as a second portion.
22. A recombinant nucleic acid for expression of an ADAMTS-12 polypeptide that comprises
   a first portion having a sequence identity of >80% with the sequence of residues 1-244 of SEQ ID NO: 15, and
   a second portion having a sequence identity of >80% with the sequence of residues 245-547 of SEQ ID NO: 15.
23. The recombinant nucleic acid of any one of clauses 21 or 22, wherein the first portion comprises residues 1-244 of SEQ ID NO: 15, and/or wherein the second portion amino acid sequence comprises residues 245-547 of SEQ ID NO: 15.
24. A recombinant nucleic acid for expression of an ADAMTS-12 polypeptide, wherein the recombinant nucleic acid encodes for a recombinant polypeptide that comprises
   a first portion having an amino acid sequence that aligns with a segment of an ADAMTS-12 prodomain amino acid sequence from a first species with a Needleman-Wunsch score greater than 800 when BLOSUM62 matrix, a gap opening penalty of 11, and a gap extension penalty of 1 are used; and
   a second portion having an amino acid sequence that aligns with a segment of an ADAMTS12 catalytic domain amino acid sequence from a second species with a Needleman-Wunsch score greater than 1000 when BLOSUM62 matrix, a gap opening penalty of 11, and a gap extension penalty of 1 are used
   wherein optionally the first species is rat and the second species is human.
25. The recombinant nucleic acid of any one of clauses 21 to 24, wherein the second portion comprises an E393Q mutation.
26. The recombinant nucleic acid of any of clauses 16 to 25, wherein the encoded polypeptide or the second portion thereof is suited to cleave a peptide comprising standard residues 1-15 of the amino acid sequence of SEQ ID NO: 4, preferably with a kcat/KM of at least 20% of a corresponding kcat/KM of human ADAMTS-7 or human ADAMTS-12.
27. A recombinant polypeptide encoded by the recombinant nucleic acid according to any of clauses 16 to 26, or a fragment of that recombinant polypeptide, wherein said recombinant polypeptide or fragment thereof is suited to cleave a peptide substrate comprising standard residues 1-15 of SEQ ID NO: 4.
28. A peptide substrate for ADAMTS-7 and/or ADAMTS-12, the peptide substrate comprising
   residues 1-15 of the amino acid sequence of SEQ ID NO: 4, or
   residues 1-15 of the amino acid sequence of SEQ ID NO: 5, or
   residues 1-13 of the amino acid sequence of SEQ ID NO: 8, or
   a fragment of any of the sequences according to a), b) or c), the fragment comprising the amino acid sequence EL.
29. The peptide substrate of clause 28, wherein the peptide substrate comprises a first moiety conjugated to a residue that is N-terminal to sequence fragment EL comprised within SEQ ID No. 4, 5, or 8 or the fragment thereof, and a second moiety conjugated to a residue that is C-terminal to said sequence fragment EL.
30. The peptide substrate of clause 29, wherein the first moiety comprises a fluorophore and the second moiety comprises a quencher, or wherein the first moiety comprises a quencher and the second moiety comprises a fluorophore.
31. A method for the identification or characterization of an ADAMTS-7 and/or ADAMTS-12 modulator comprising the steps of
   contacting a recombinant polypeptide or a fragment thereof according to clause 27 with at least one test compound;
   contacting said recombinant polypeptide or fragment thereof with a peptide substrate according to any one of clauses 28 to 30, wherein the peptide substrate comprises a fluorophore and a quencher; and
   detecting fluorescence as a measure for the activity of said recombinant polypeptide or a fragment thereof.
32. A modulator of ADAMTS-7 and/or ADAMTS-12 identified by a method according to clause 31 for use in the treatment of coronary artery disease (CAD), peripheral vascular disease (PAD) and myocardial infarction (MI).
33. A method of producing a recombinant polypeptide according to clause 32, the method comprising
   cultivating a recombinant host cell comprising a recombinant nucleic acid according to any of clauses 16 to 26
   recovering the recombinant polypeptide of a fragment thereof according to clause 34.
34. Kit of parts comprising at least one recombinant nucleic acid according to any of clauses 16 to 26 or at least one polypeptide according to clause 27 and a peptide substrate according to any of clauses 28 to 30.

1. A compound of formula (I): 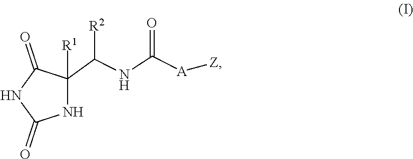
wherein
R¹ is selected from the group consisting of hydrogen, (C₁-C₆)-alkyl, (C₃-C₆)-cycloalkyl, 5- to 6-membered heterocycloalkyl, 5- to 6-membered heteroaryl, and phenyl,
wherein said (C₁-C₆)-alkyl, (C₃-C₆)-cycloalkyl, 5- to 6-membered heterocycloalkyl, 5- to 6-membered heteroaryl or phenyl is optionally substituted with one or two groups independently selected from the group consisting of cyano, halogen, amino, hydroxy, oxo, (C₁-C₃)-alkyl, (C₁-C₄)-alkoxy, (C₁-C₄)-alkylcarbonyl, mono-(C₁-C₄)-alkylamino, di-(C₁-C₄)-alkylamino, (C₁-C₄)-alkylsulfonyl, and (C₃-C₆)-cycloalkyl,
wherein each said (C₁-C₃)-alkyl, (C₃-C₆)-cycloalkyl, and (C₁-C₄)-alkoxy is optionally substituted with up to 5 fluorine atoms;
R² is hydrogen or (C₁-C₄)-alkyl,
wherein said (C₁-C₄)-alkyl is optionally substituted with up to five fluorine atoms;
A is 5-membered heteroaryl,
wherein said 5-membered heteroaryl is optionally substituted with one, two or three groups independently selected from halogen, cyano, hydroxy, methylsulfonyl, (C₃-C₆)-cycloalkyl, (C₁-C₄)-alkyl, and (C₁-C₄)-alkoxy,
wherein each said (C₃-C₆)-cycloalkyl, (C₁-C₄)-alkyl, and (C₁-C₄)-alkoxy is optionally substituted with up to three fluorine atoms; and
Z is 6- to 10-membered aryl or 5- to 10-membered heteroaryl,
wherein said 6- to 10-membered aryl or 5- to 10-membered heteroaryl is optionally substituted with one, two or three groups independently selected from halogen, cyano, hydroxy, (C₁-C₄)-alkylsulfonyl, (C₃-C₆)-cycloalkyl, (C₁-C₄)-alkyl, and (C₁-C₄)-alkoxy,
wherein each said (C₁-C₄)-alkyl and (C₁-C₄)-alkoxy is optionally substituted with (C₃-C₆)-cycloalkyl and optionally substituted with up to five fluorine atoms,
or a pharmaceutically acceptable salt thereof, solvate thereof, or a solvate of a pharmaceutically acceptable salt thereof.

2. The compound of claim 1, or a pharmaceutically acceptable salt thereof, a solvate thereof, or a solvate of a pharmaceutically acceptable salt thereof, wherein the compound is not a compound of any one of formula (Xa) to formula (Xi):
4-(4-chlorophenyl)-N-[[4-(1-methylimidazol-2-yl)-2,5-dioxo-imidazolidin-4-yl]methyl]-1H-pyrrole-2-carboxamide 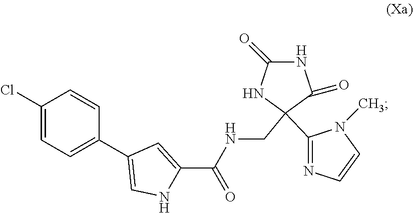 [SMILES: Cn1ccnc1C1(CNC(=O)c2cc(-c3ccc(Cl)cc3)c[nH]2)NC(=O)NC1=O]
1-(4-fluorophenyl)-N-[[4-(1-methylimidazol-2-yl)-2,5-dioxo-imidazolidin-4-yl]methyl]pyrazole-4-carboxamide 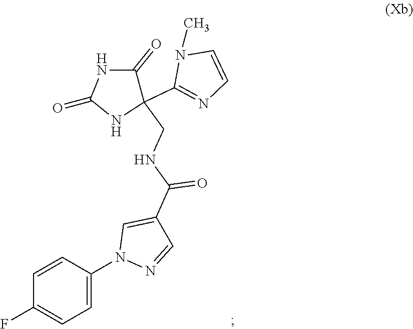 [SMILES: Cn1ccnc1C1(CNC(=O)c2cnn(-c3ccc(F)cc3)c2)NC(=O)NC1=O]
1-methyl-N-[[4-(1-methylimidazol-2-yl)-2,5-dioxo-imidazolidin-4-yl]methyl]-5-phenyl-pyrrole-2-carboxamide 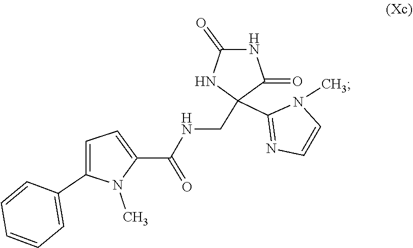 [SMILES: Cn1ccnc1C1(CNC(=O)c2ccc(-c3ccccc3)n2C)NC(=O)NC1=O]
4-(5-chloro-2-thienyl)-N-[[4-(1-methylimidazol-2-yl)-2,5-dioxo-imidazolidin-4-yl]methyl]-1H-pyrrole-3-carboxamide 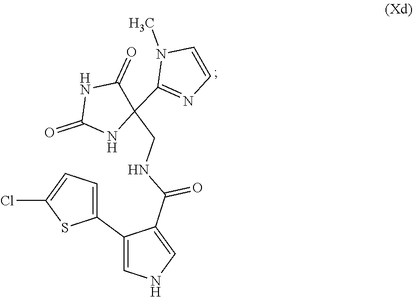 [SMILES: Cn1ccnc1C1(CNC(=O)c2c[nH]cc2-c2ccc(Cl)s2)NC(=O)NC1=O]
4-methyl-N-[[4-(1-methylimidazol-2-yl)-2,5-dioxo-imidazolidin-4-yl]methyl]-2-(2-thienyl)thiazole-5-carboxamide 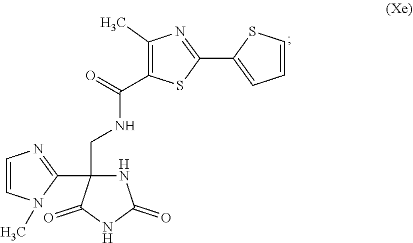 [SMILES: Cc1nc(-c2cccs2)sc1C(=O)NCC1(c2nccn2C)NC(=O)NC1=O]
N-[[4-(1-methylimidazol-2-yl)-2,5-dioxo-imidazolidin-4-yl]methyl]-2-(4-pyridyl)thiazole-5-carboxamide 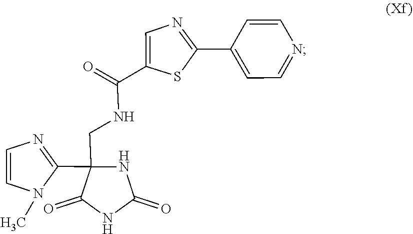 [SMILES: Cn1ccnc1C1(CNC(=O)c2cnc(-c3ccncc3)s2)NC(=O)NC1=O]
5-methyl-N-[[4-(1-methylimidazol-2-yl)-2,5-dioxo-imidazolidin-4-yl]methyl]-1-(4-pyridyl)pyrazole-4-carboxamide 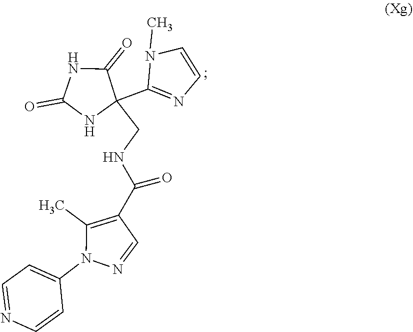 [SMILES: Cc1c(C(=O)NCC2(c3nccn3C)NC(=O)NC2=O)cnn1-c1ccncc1]
N-[[4-(1-methylimidazol-2-yl)-2,5-dioxo-imidazolidin-4-yl]methyl]-5-(1H-pyrazol-3-yl)thiophene-2-carboxamide 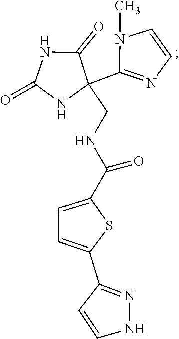 [SMILES: Cn1ccnc1C1(CNC(=O)c2ccc(-c3cc[nH]n3)s2)NC(=O)NC1=O]
and
N-[[4-(1-methylimidazol-2-yl)-2,5-dioxo-imidazolidin-4-yl]methyl]-5-phenyl-1H-pyrazole-3-carboxamide

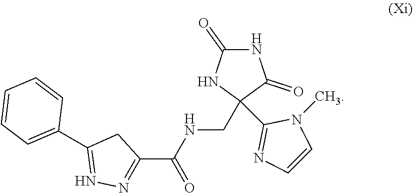 [SMILES: Cn1ccnc1C1(CNC(=O)C2=NN=C(c3ccccc3)C2)NC(=O)NC1=O]

3. The compound of claim 1, wherein
R¹ is selected from the group consisting of hydrogen, (C₁-C₆)-alkyl, (C₃-C₆)-cycloalkyl, 5- to 6-membered heterocycloalkyl, 5- to 6-membered heteroaryl, and phenyl,
wherein said (C₁-C₆)-alkyl (C₃-C₆)-cycloalkyl, 5- to 6-membered heterocycloalkyl, 5- to 6-membered heteroaryl, or phenyl is optionally substituted with one or two groups independently selected from the group consisting of cyano, halogen, amino, hydroxy, oxo, C₁-C₃-alkyl, (C₁-C₄)-alkoxy, (C₁-C₄)-alkylcarbonyl, mono-(C₁-C₄)-alkylamino, di-(C₁-C₄)-alkylamino, (C₁-C₄)-alkylsulfonyl, and (C₃-C₆)-cycloalkyl,
wherein each said (C₁-C₃)-alkyl, (C₃-C₆)-cycloalkyl, and (C₁-C₄)-alkoxy is optionally substituted with up to 5 fluorine atoms;
R² hydrogen or (C₁-C₄)-alkyl,
wherein said (C₁-C₄)-alkyl is optionally substituted with up to five fluorine atoms,
A is 5-membered aza-heteroaryl;
wherein said 5-membered aza-heteroaryl is optionally substituted with one, two, or three groups independently selected from the group consisting of halogen, cyano, hydroxy, methylsulfonyl, (C₃-C₆)-cycloalkyl, (C₁-C₄)-alkyl, and (C₁-C₄)-alkoxy,
wherein each said (C₁-C₄)-alkyl, (C₁-C₄)-alkoxy is optionally substituted with up to three fluorine atoms; and
Z is selected from the group consisting of phenyl and 5- to 6-membered heteroaryl,
optionally substituted, with one, two or three groups independently selected from halogen, cyano, hydroxy, (C₁-C₄)-alkylsulfonyl, (C₃-C₆)-cycloalkyl, (C₁-C₄)-alkyl, and (C₁-C₄)-alkoxy,
wherein each said (C₁-C₄)-alkyl and (C₁-C₄)-alkoxy is optionally substituted with (C₃-C₆)-cycloalkyl and optionally substituted with up to five fluorine atoms,
or a pharmaceutically acceptable salt thereof, a solvate thereof, or a solvate of a pharmaceutically acceptable salt thereof.

4. The compound of claim 1, wherein
R¹ is selected from the group consisting of hydrogen, (C₁-C₆)-alkyl, (C₃-C₆)-cycloalkyl, 5- to 6-membered heterocycloalkyl, 5- to 6-membered heteroaryl, and phenyl,
wherein said (C₁-C₆)-alkyl (C₃-C₆)-cycloalkyl, 5- to 6-membered heterocycloalkyl, 5- to 6-membered heteroaryl or phenyl is optionally substituted, with one or two groups independently selected from cyano, halogen, amino, hydroxy, oxo, C₁-C₃-alkyl, (C₁-C₄)-alkoxy, (C₁-C₄)-alkylcarbonyl, mono-(C₁-C₄)-alkylamino, di-(C₁-C₄)-alkylamino, (C₁-C₄)-alkylsulfonyl, and (C₃-C₆)-cycloalkyl,
wherein each said C₁-C₃-alkyl, (C₃-C₆)-cycloalkyl and (C₁-C₄)-alkoxy is optionally substituted with up to 5 fluorine atoms;
R² is hydrogen or methyl;
A is selected from the group consisting of triazolyl, oxazolyl, isoxazolyl, pyrazolyl, thiazolyl, 1,2,4-oxadiazolyl, and 1,3,4-oxadiazolyl,
optionally substituted with one, two or three groups independently selected from halogen, cyano, hydroxy, methylsulfonyl, (C₃-C₆)-cycloalkyl, (C₁-C₄)-alkyl and (C₁-C₄)-alkoxy,
wherein each said (C₁-C₄)-alkyl, (C₁-C₄)-alkoxy is optionally substituted with up to three fluorine atoms; and
Z is selected from the group consisting of phenyl and 5- to 6-membered heteroaryl,
optionally substituted, with one, two or three groups independently selected from halogen, cyano, hydroxy, methylsulfonyl, (C₃-C₆)-cycloalkyl, (C₁-C₄)-alkyl, and (C₁-C₄)-alkoxy,
wherein each said (C₁-C₄)-alkyl and (C₁-C₄)-alkoxy is optionally substituted with (C₃-C₆)-cycloalkyl and optionally substituted with up to five fluorine atoms,
or a pharmaceutically acceptable salt thereof, a solvate thereof, or a solvate of a pharmaceutically acceptable salt thereof.

5. The compound of claim 1, wherein
R¹ is selected from the group consisting of hydrogen, (C₁-C₆)-alkyl, (C₃-C₆)-cycloalkyl, 5- to 6-membered heterocycloalkyl, 5- to 6-membered heteroaryl, and phenyl,
wherein said (C₁-C₆)-alkyl, (C₃-C₆)-cycloalkyl, 5- to 6-membered heterocycloalkyl, 5- to 6-membered heteroaryl or phenyl is optionally substituted, with one or two groups independently selected from cyano, halogen, amino, hydroxy, oxo, C₁-C₃-alkyl, (C₁-C₄)-alkoxy, (C₁-C₄)-alkylcarbonyl, mono-(C₁-C₄)-alkylamino, di-(C₁-C₄)-alkylamino, (C₁-C₄)-alkylsulfonyl, and (C₃-C₆)-cycloalkyl,
wherein each said C₁-C₃-alkyl, (C₃-C₆)-cycloalkyl, and (C₁-C₄)-alkoxy is optionally substituted with up to 5 fluorine atoms;
R² is hydrogen or methyl;
A is triazolyl; and
optionally substituted with one, two or three groups independently selected from halogen, cyano, hydroxy, methylsulfonyl, (C₃-C₆)-cycloalkyl, (C₁-C₄)-alkyl, and (C₁-C₄)-alkoxy,
wherein each said (C₁-C₄)-alkyl, and (C₁-C₄)-alkoxy is optionally substituted with up to three fluorine atoms,
Z is selected from the group consisting of phenyl and 5 to 6 membered heteroaryl,
optionally substituted, with one, two or three groups independently selected from halogen, cyano, hydroxy, methylsulfonyl, (C₃-C₆)-cycloalkyl, (C₁-C₄)-alkyl, and (C₁-C₄)-alkoxy,
wherein each said (C₁-C₄)-alkyl and (C₁-C₄)-alkoxy is optionally substituted with (C₃-C₆)-cycloalkyl and optionally substituted with up to five fluorine atoms,
or a pharmaceutically acceptable salt thereof, a solvate thereof, or a solvate of a pharmaceutically acceptable salt thereof.

6. The compound of claim 1, wherein the compound is selected from the group consisting of:
N-{[(4R)-4-cyclopropyl-2,5-dioxoimidazolidin-4-yl]methyl}-2-(2,4-difluorophenyl)-2H-1,2,3-triazole-4-carboxamide 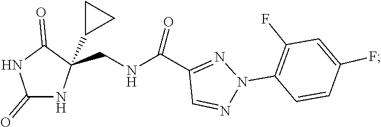 [SMILES: O=C1NC(=O)[C@](CNC(=O)c2cnn(-c3ccc(F)cc3F)n2)(C2CC2)N1]
N-{[(4R)-4-cyclopropyl-2,5-dioxoimidazolidin-4-yl]methyl}-2-(4-fluorophenyl)-2H-1,2,3-triazole-4-carboxamide 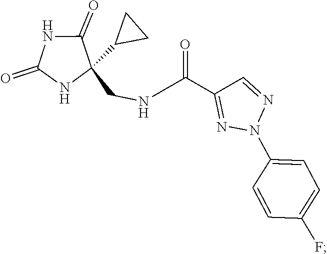 [SMILES: O=C1NC(=O)[C@](CNC(=O)c2cnn(-c3ccc(F)cc3)n2)(C2CC2)N1]
2-(3-chloro-4-fluorophenyl)-N-{[(4R)-4-cyclopropyl-2,5-dioxoimidazolidin-4-yl]methyl}-2H-1,2,3-triazole-4-carboxamide 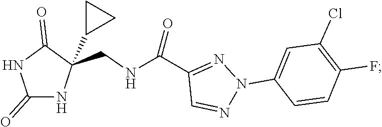 [SMILES: O=C1NC(=O)[C@](CNC(=O)c2cnn(-c3ccc(F)c(Cl)c3)n2)(C2CC2)N1]
ent-N-[(2,5-dioxo-4-(propan-2-yl)imidazolidin-4-yl)methyl]-2-(4-fluorophenyl)-2H-1,2,3-triazole-4-carboxamide 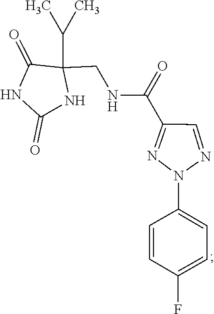 [SMILES: CC(C)C1(CNC(=O)c2cnn(-c3ccc(F)cc3)n2)NC(=O)NC1=O]
ent-N-[(4-cyclobutyl-2,5-dioxoimidazolidin-4-yl)methyl]-2-(4-fluorophenyl)-2H-1,2,3-triazole-4-carboxamide 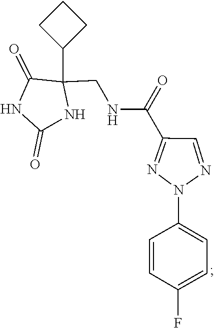 [SMILES: O=C1NC(=O)C(CNC(=O)c2cnn(-c3ccc(F)cc3)n2)(C2CCC2)N1]
ent-N-[(4-cyclobutyl-2,5-dioxoimidazolidin-4-yl)methyl]-2-(3,4,5-trifluorophenyl)-2H-1,2,3-triazole-4-carboxamide 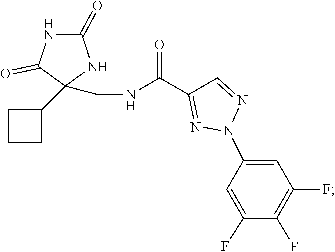 [SMILES: O=C1NC(=O)C(CNC(=O)c2cnn(-c3cc(F)c(F)c(F)c3)n2)(C2CCC2)N1]
ent-N-[(4-cyclobutyl-2,5-dioxoimidazolidin-4-yl)methyl]-2-(3-fluorophenyl)-2H-1,2,3-triazole-4-carboxamide 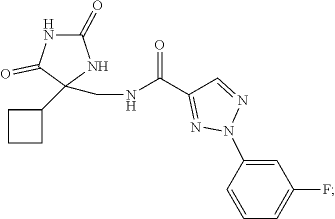 [SMILES: O=C1NC(=O)C(CNC(=O)c2cnn(-c3cccc(F)c3)n2)(C2CCC2)N1]
and
ent-N-[(4-cyclobutyl-2,5-dioxoimidazolidin-4-yl)methyl]-2-(3,4-difluorophenyl)-2H-1,2,3-triazole-4-carboxamide 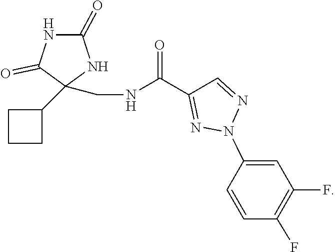 [SMILES: O=C1NC(=O)C(CNC(=O)c2cnn(-c3ccc(F)c(F)c3)n2)(C2CCC2)N1]
or a pharmaceutically acceptable salt thereof, a solvate thereof, or a solvate of a pharmaceutically acceptable salt thereof.

7-10. (canceled)

11. A pharmaceutical composition comprising the compound of claim 1, a pharmaceutically acceptable salt thereof, a solvate thereof, or a solvate of a pharmaceutically acceptable salt thereof, and one or more pharmaceutically acceptable excipients.

12. The pharmaceutical composition of claim 11, further comprising one or more additional active ingredients.

13-14. (canceled)

15. A method for the treatment or prevention of diseases, comprising administering to mammal in need thereof a therapeutically effective amount of the compound of claim 1.

16. The method of claim 15, wherein the disease is a heart disease, vascular disease, cardiovascular disease, lung disease, inflammatory disease, fibrotic disease, metabolic disease, or cardiometabolic disease.

17. The method of claim 16, wherein the disease is atherosclerosis, coronary artery disease, peripheral vascular disease, arterial occlusive disease, or a post-surgery complication of any of the foregoing.

18. The method of claim 17, wherein the disease is restenosis after angioplasty.

19. The method of claim 15, wherein the mammal is a human.

20. The pharmaceutical composition of claim 11, further comprising one or more therapeutic agents selected from the group consisting of angiotensin-converting enzyme inhibitors, angiotensin-receptor blockers, mineralocorticoid-receptor antagonists, endothelin antagonists, renin inhibitors, calcium blockers, beta-receptor blockers, vasopeptidase inhibitors, sodium-glucose-transport-antagonists, metformin, pioglitazones and dipeptidyl-peptidase-IV inhibitors.

21. A method for treatment or prevention of atherosclerosis or an atherosclerosis-related disease, comprising administering to a mammal in need thereof a therapeutically effective amount of the composition of claim 11.

22. The method of claim 21, wherein the treatment or prevention is for an atherosclerosis-related disease selected from the group consisting of coronary artery disease, peripheral vascular disease, arterial occlusive disease, and a post-surgery complication of any of the foregoing.

23. The method of claim 22, wherein the atheroschlerosis-related disease is restenosis after angioplasty.

24. The method of claim 21, wherein the mammal is a human.
